(19) United States
(12) Patent Application Publication
(10) Pub. No.: US 2025/0304994 A1
(43) Pub. Date: Oct. 2, 2025

(54) Circular RNA Compositions and Methods

(71) Applicant: Orna Therapeutics, Inc., Watertown, MA (US)

(72) Inventors: Robert Alexander Wesselhoeft, Somerville, MA (US); Kristen Ott, Watertown, MA (US); Huan Shu, Watertown, MA (US); Varun Shivashankar, Watertown, MA (US)

(73) Assignee: Orna Therapeutics, Inc., Watertown, MA (US)

(21) Appl. No.: 19/234,626

(22) Filed: Jun. 11, 2025

Related U.S. Application Data
(63) Continuation of application No. PCT/US23/84046, filed on Dec. 14, 2023.
(60) Provisional application No. 63/387,600, filed on Dec. 15, 2022.
(60) Provisional application No. 63/387,559, filed on Dec. 15, 2022.

(51) Int. Cl. C12N 15/85; A61K 40/11; A61K 40/31; A61K 40/42; C12N 5/0783; C12N 13/00

(52) U.S. Cl. CPC C12N 15/85; A61K 40/11; A61K 40/31; A61K 40/4202; A61K 40/4211; C12N 5/0636; C12N 13/00; C12N 2510/00; C12N 2840/203

(57) ABSTRACT

Circular RNA, along with related compositions and methods are described herein. In some embodiments, the inventive circular RNA comprises intron segments, spacers, an IRES, duplex forming regions, and an expression sequence. In some embodiments, circular RNA of the invention has improved expression, functional stability, immunogenicity, ease of manufacturing, and/or half-life when compared to linear RNA. In some embodiments, the disclosed methods and constructs result in improved translation when compared to existing RNA approaches. In some embodiments, the disclosed methods and constructs result in improved circularization efficiency, splicing efficiency, and/or purity when compared to existing RNA circularization approaches.

### CROSS-REFERENCE TO RELATED APPLICATIONS

This application is a continuation of PCT/US2023/084046, filed Dec. 14, 2023, which claims the benefit of priority of U.S. Provisional Application No. 63/387,600, filed Dec. 15, 2022, and U.S. Provisional Application No. 63/387,559, filed Dec. 15, 2022, each of which is incorporated by reference herein in its entirety for any purpose.

### SEQUENCE LISTING

This application is filed with a Sequence Listing in electronic format. The Sequence Listing is provided as a file entitled “01318-0009-00PCT_SL.xml” created on Dec. 14, 2023, which is 30,429,268 bytes in size. The information in the electronic format of the sequence listing is incorporated herein by reference in its entirety.

### BACKGROUND OF THE INVENTION

Conventional gene therapy involves the use of DNA for insertion of desired genetic information into host cells. The DNA introduced into the cell is usually integrated to a certain extent into the genome of one or more transfected cells, allowing for long-lasting action of the introduced genetic material in the host. While there may be substantial benefits to such sustained action, integration of exogenous DNA into a host genome may also have many deleterious effects. For example, it is possible that the introduced DNA will be inserted into an intact gene, resulting in a mutation which impedes or even totally eliminates the function of the endogenous gene. Thus, gene therapy with DNA may result in the impairment of a vital genetic function in the treated host, such as e.g., elimination or deleteriously reduced production of an essential enzyme or interruption of a gene critical for the regulation of cell growth, resulting in unregulated or cancerous cell proliferation. In addition, with conventional DNA based gene therapy it is necessary for effective expression of the desired gene product to include a strong promoter sequence, which again may lead to undesirable changes in the regulation of normal gene expression in the cell. It is also possible that the DNA based genetic material will result in the induction of undesired anti-DNA antibodies, which in turn, may trigger a possibly fatal immune response. Gene therapy approaches using viral vectors can also result in an adverse immune response. In some circumstances, the viral vector may even integrate into the host genome. In addition, production of clinical grade viral vectors is also expensive and time consuming. Targeting delivery of the introduced genetic material using viral vectors can also be difficult to control. Thus, while DNA based gene therapy has been evaluated for delivery of secreted proteins using viral vectors, these approaches may be limited for these various reasons.

In contrast to DNA, the use of RNA as a gene therapy agent is substantially safer because RNA does not involve the risk of being stably integrated into the genome of the transfected cell, thus eliminating the concern that the introduced genetic material will disrupt the normal functioning of an essential gene, or cause a mutation that results in deleterious or oncogenic effects, and extraneous promoter sequences are not required for effective translation of the encoded protein, again avoiding possible deleterious side effects. In addition, it is not necessary for mRNA to enter the nucleus to perform its function, while DNA must overcome this major barrier.

Circular RNA is useful in the design and production of stable forms of RNA. The circularization of an RNA molecule provides an advantage to the study of RNA structure and function, especially in the case of molecules that are prone to folding in an inactive conformation. Circular RNA can also be particularly interesting and useful for in vivo applications, especially in the research area of RNA-based control of gene expression and therapeutics, including protein replacement therapy and vaccination.

The present disclosure provides methods and compositions for the manufacture and optimization of circularized RNAs via engineering of the sequences for the precursor linear RNA and ultimately the circular RNA along with methods of treating a subject in need using the disclosed circular RNA polynucleotides.

### SUMMARY

Precursor RNA polynucleotides, circular RNA polynucleotides (oRNA™) pharmaceutical compositions comprising oRNAs, and related methods are described herein.

Disclosed herein, in certain embodiments, is a circular RNA polynucleotide (oRNA) comprising a translation initiation element (TIE), wherein the TIE comprises a sequence having at least 85% sequence identity to a sequence set forth in any one of SEQ ID NOS: 14067-24829 (GIRES-1 through GIRES-10762), or a fragment thereof, optionally barcoded with a barcode sequence selected from SEQ ID NOs: 3304-14066 (e.g., the IRES of SEQ ID NO: 14067 is barcoded with the barcode sequence of SEQ ID NO: 3304, the IRES of SEQ ID NO: 14068 is barcoded with the barcode sequence of SEQ ID NO: 3305, the IRES of SEQ ID NO: 14069 is barcoded with the barcode sequence of SEQ ID NO: 3306, and sequentially thereon). In some embodiments, the TIE comprises a consensus sequence as set forth in the Table of Exemplary Consensus Sequences herein (SEQ ID NOs: 24867-24892, Table A), wherein N is any nucleotide (e.g., pursuant to IUPAC). In some embodiments, the TIE comprises at least 100 nucleotides, at least 200 nucleotides, at least 300 nucleotides, at least 400 nucleotides, at least 500 nucleotides, at least 600 nucleotides, or at least 700 nucleotides (e.g., contiguous nucleotides) of said consensus sequence.

Disclosed herein, in certain embodiments, is a circular RNA polynucleotide (oRNA), comprising a core functional element, and a pharmaceutically acceptable salt, buffer, diluent, or combination thereof; wherein the core functional element comprises a translation initiation element (TIE), wherein the TIE comprises a sequence having at least 85% sequence identity to a sequence set forth in any one of SEQ ID NOS: 14067-24829, or a fragment thereof.

Disclosed herein, in certain embodiments, is a circular RNA polynucleotide (oRNA), comprising a core functional element, and a pharmaceutically acceptable salt, buffer, diluent, or combination thereof; wherein the core functional element comprises a translation initiation element (TIE), wherein the TIE comprises a sequence having at least 85% sequence identity to a sequence set forth in any one of SEQ ID NOs: 793, 876, 1017, 1216, and 3291, wherein the oRNA is capable of expressing a therapeutic protein in a T cell.

Disclosed herein, in certain embodiments, is a circular RNA polynucleotide (oRNA), comprising a core functional element, and a pharmaceutically acceptable salt, buffer, diluent, or combination thereof; wherein the core functional element comprises a translation initiation element (TIE), wherein the TIE comprises at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, wherein the oRNA is capable of expressing a therapeutic protein in a T cell.

Disclosed herein, in certain embodiments, is a circular RNA polynucleotide (oRNA), comprising a core functional element, and a pharmaceutically acceptable salt, buffer, diluent, or combination thereof; wherein the core functional element comprises a translation initiation element (TIE), wherein the TIE comprises at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, wherein the oRNA is capable of expressing a therapeutic protein in a T cell.

Also disclosed herein, in certain embodiments, is a precursor RNA polynucleotide capable of producing the circular RNA provided herein.

In certain embodiments, the TIE comprises an internal ribosome entry site (IRES). In certain embodiments, the IRES is in whole or in part from an untranslated region (UTR). In certain embodiments, the IRES has at least 90% identity to a sequence set forth in any one of SEQ ID NOS: 14067-24829 (GIRES-1 through GIRES-10762). In certain embodiments, the IRES has at least 95% identity to a sequence set forth in any one of SEQ ID NOS: 14067-24829. In certain embodiments, the IRES has at least 98% identity to a sequence set forth in any one of SEQ ID NOS: 14067-24829. In certain embodiments, the IRES has at least 99% identity to a sequence set forth in any one of SEQ ID NOS: 14067-24829. In certain embodiments, the IRES comprises a sequence set forth in any one of SEQ ID NOS: 14067-24829.

In certain embodiments, the TIE comprises an internal ribosome entry site (IRES). In certain embodiments, the IRES is in whole or in part from an untranslated region (UTR). In certain embodiments, the IRES sequence has at least 90% sequence identity to a sequence set forth in any one of SEQ ID NO: 793, 876, 1017, 1216, and 3291. In certain embodiments, the IRES sequence has at least 95% sequence identity to a sequence set forth in any one of SEQ ID NO: 793, 876, 1017, 1216, and 3291. In certain embodiments, the IRES sequence has at least 98% sequence identity to a sequence set forth in any one of SEQ ID NO: 793, 876, 1017, 1216, and 3291. In certain embodiments, the IRES sequence has at least 99% sequence identity to a sequence set forth in any one of SEQ ID NO: 793, 876, 1017, 1216, and 3291. In certain embodiments, the IRES sequence comprises a sequence set forth in any one of SEQ ID NO: 793, 876, 1017, 1216, and 3291. In certain embodiments, the IRES sequence has at least 90% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the IRES sequence has at least 95% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the IRES sequence has at least 98% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the IRES sequence has at least 99% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the IRES sequence comprises a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the IRES sequence has at least 90% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the IRES sequence has at least 95% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the IRES sequence has at least 98% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the IRES sequence has at least 99% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the IRES sequence comprises a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

In certain embodiments, the precursor RNA polynucleotide further comprises an accessory element. In certain embodiments, the accessory element comprises a miRNA binding site or a fragment thereof, a restriction site or a fragment thereof, an RNA editing motif or a fragment thereof, a zip code element or a fragment thereof, an RNA trafficking element or a fragment thereof, an endonuclease site or a fragment thereof, or a combination thereof. In certain embodiments, the accessory element comprises a binding domain to an IRES transacting factor (ITAF) and/or a translation initiation factor. In certain embodiments, the binding domain comprises a polyA region, a polyC region, a polyAC region, a polypyrimidine tract, or a combination or variant thereof. In certain embodiments, the ITAF comprises a poly(rC)-binding protein 1 (PCBP1), PCBP2, PCBP3, PCBP4, poly(A)-binding protein 1 (PABP1), polypyrimidine-tract binding protein (PTB), Argonaute protein family member, HNRNPK (heterogeneous nuclear ribonucleoprotein K protein), or La protein, or a fragment or combination thereof. In certain embodiments, the core functional element further comprises a coding sequence and, optionally, a termination sequence located downstream to the coding sequence. In certain embodiments, the coding sequence is located downstream to the IRES. In certain embodiments, the coding sequence is located upstream to the IRES. In certain embodiments, the termination sequence is a stop codon or a stop cassette. In certain embodiments, the stop cassette comprises one or more stop codons in two or more open reading frames.

In certain embodiments, the precursor RNA polynucleotide comprises: (a) a 5′ enhanced intron element, (b) a 5′ enhanced exon element, (c) the core functional element, (d) a 3′ enhanced exon element, and (e) a 3′ enhanced intron element. In certain embodiments, elements (a)-(e) are arranged in order from (a) to (e). In certain embodiments, the 5′ enhanced exon element and/or the 3′ enhanced exon element are each comprised within the core functional element. In certain embodiments, the 5′ enhanced exon element and/or the 3′ enhanced exon element are each comprised within the coding sequence.

In certain embodiments, the 5′ enhanced intron element comprises a 3′ intron segment. In certain embodiments, the 3′ intron segment further comprises a first or a first and a second nucleotides of a 3′ group I intron splice site dinucleotide. In certain embodiments, the 3′ intron segment is located at the 3′ end of the 5′ enhanced intron element. In certain embodiments, the 5′ enhanced intron element comprises a leading untranslated sequence located at the 5′ end. In certain embodiments, the leading untranslated sequence comprises a spacer. In certain embodiments, the leading untranslated sequence comprises the last nucleotide of a transcription start site. In certain embodiments, the leading untranslated sequence comprises 1 to 100 additional nucleotides. In certain embodiments, the 5′ enhanced intron element comprises a 5′ affinity sequence. In certain embodiments, the 5′ affinity sequence comprises a polyA, polyAC, or polypyrimidine sequence. In certain embodiments, the 5′ affinity sequence comprises 10 to 100 nucleotides. In certain embodiments, the 5′ enhanced intron element comprises a 5′ external spacer sequence. In certain embodiments, the 5′ external spacer sequence is located between the 5′ affinity sequence and the 3′ intron segment. In certain embodiments, the 5′ external spacer sequence has a length of about 6 to 60 nucleotides. In certain embodiments, the 5′ external spacer sequence comprises or consists of a sequence selected from SEQ ID NOs: 3094-3152.

In certain embodiments, the 5′ enhanced intron element comprises: (a) a leading untranslated sequence; (b) a 5′ affinity sequence; (c) a 5′ external spacer sequence; and (d) a 3′ intron segment including the first nucleotide of a 3′ Group I intron splice site; wherein the leading untranslated sequence comprises the last nucleotide of a transcription start site and 1 to 100 nucleotides. In certain embodiments, (a)-(d) are arranged in the order from (a) to (d). In certain embodiments, the 5′ enhanced intron element comprises: (a) a leading untranslated sequence; (b) a 5′ external spacer sequence; (c) a 5′ affinity sequence; and (d) a 3′ intron segment including the first nucleotide of a 3′ group I splice site; wherein the leading untranslated sequence comprises the last nucleotide of a transcription start site and 1 to 100 nucleotide. In certain embodiments, (a)-(d) are arranged in the order from (a) to (d). In certain embodiments, the 5′ enhanced intron element comprises: (a) a leading untranslated sequence; (b) a 5′ external spacer sequence; (c) a 5′ affinity sequence; and a 3′ intron segment including the first and second nucleotides of a 3′ Group I splice site; wherein the leading untranslated sequence comprises the last nucleotide of a transcription start site and 1 to 100 nucleotide; and wherein the 5′ enhanced exon element comprises a 3′ exon segment lacking the second nucleotide of a 3′ group I splice site dinucleotide. In certain embodiments, (a)-(d) are arranged in the order from (a) to (d).

In certain embodiments, the 5′ enhanced exon element comprises a 3′ exon segment. In certain embodiments, the 3′ exon segment further comprises the second nucleotide of a 3′ group I intron splice site dinucleotide. In certain embodiments, the 3′ exon segment comprises 1 to 100 natural nucleotides derived from a natural exon. In certain embodiments, the natural exon is derived from a Group I intron containing gene or a fragment thereof. In certain embodiments, the natural exon derived from an anabaena bacterium, T4 phage virus, twort bacteriophage, tetrahymena, or azoarcus bacterium. In certain embodiments, the 5′ enhanced exon element comprises a 5′ internal spacer sequence located downstream from the 3′ exon segment. In certain embodiments, the 5′ internal spacer sequence is about 6 to 60 nucleotides in length. In certain embodiments, the 5′ internal spacer sequence comprises or consists of a sequence selected from SEQ ID NOs: 3094-3152.

In certain embodiments, the 5′ enhanced exon element comprises in the following order: (a) a 3′ exon segment including the second nucleotide of a 3′ group I intron splice site dinucleotide; and (b) a 5′ internal spacer sequence, wherein the 3′ exon segment comprises 1 to 100 natural nucleotides derived from a natural exon. In certain embodiments, the 5′ enhanced exon element comprises in the following order: (a) a 3′ exon segment; and (b) a 5′ internal spacer sequence, wherein the 3′ exon segment comprises 1 to 100 natural nucleotides derived from a natural exon; and wherein the 5′ enhanced intron element comprises a 3′ intron segment comprising the first and second nucleotides of a 3′ group I splice site dinucleotide.

In certain embodiments, the 3′ enhanced exon element comprises a 5′ exon segment. In certain embodiments, the 5′ exon segment comprises the first nucleotide of a 5′ group I intron segment. In certain embodiments, the 5′ exon segment further comprises 1 to 100 nucleotides derived from a natural exon. In certain embodiments, the natural exon is derived from a Group I intron containing gene or a fragment thereof. In certain embodiments, the 3′ enhanced exon element comprises a 3′ internal spacer sequence. In certain embodiments, the 3′ internal spacer sequence is located between the termination sequence and the 5′ exon segment. In certain embodiments, the 3′ internal spacer is about 6 to 60 nucleotides in length. In certain embodiments, the 3′ internal spacer comprises or consists of a sequence selected from SEQ ID NOs: 3094-3152. In certain embodiments, the 3′ enhanced exon element comprises: (a) a 3′ internal spacer sequence; and (b) a 5′ exon segment including the first nucleotide of a 5′ group I intron splice site dinucleotide, wherein the 5′ exon segment comprises 1 to 100 nucleotides derived from a natural exon. In certain embodiments, the 3′ enhanced exon element comprises: (a) a 3′ internal spacer sequence; and (b) a 5′ exon segment, wherein the 5′ exon segment comprises 1 to 100 nucleotides derived from a natural exon; wherein the 3′ enhanced intron element comprises a 5′ intron segment comprising the first and second nucleotide of a 5′ group I intron splice site dinucleotide.

In certain embodiments, the 3′ enhanced intron element comprises a 5′ intron segment. In certain embodiments, the 5′ intron segment comprises a second nucleotide of a 5′ group I intron splice site dinucleotide. In certain embodiments, the 3′ enhanced intron element comprises a trailing untranslated sequence located at the 3′ end of the 5′ intron. In certain embodiments, the trailing untranslated sequence comprises 3 to 12 nucleotides. In certain embodiments, the 3′ enhanced intron segment comprises a 3′ external spacer sequence. In certain embodiments, the 3′ external spacer sequence is located between the 5′ intron segment and trailing untranslated sequence. In certain embodiments, the 3′ external spacer sequence has a length of 6 to 60 nucleotides in length. In certain embodiments, the 3′ external spacer sequence comprises or consists of a sequence selected SEQ ID NOs: 3094-3152. In certain embodiments, the 3′ enhanced intron element comprises a 3′ affinity sequence. In certain embodiments, the 3′ affinity sequence is located between the 3′ external spacer sequence and the trailing untranslated sequence. In certain embodiments, the 3′ affinity sequence comprises a polyA, polyAC, or polypyrimidine sequence. In certain embodiments, the affinity sequence comprises 10 to 100 nucleotides.

In certain embodiments, the 5′ enhanced intron element further comprises a 5′ external duplex sequence; wherein the 3′ enhanced intron element further comprises a 3′ external duplex sequence. In certain embodiments, the 5′ external duplex sequence and 3′ external duplex sequence are fully or partially complementary to each other. In certain embodiments, the 5′ external duplex sequence comprises fully synthetic or partially synthetic nucleotides. In certain embodiments, the 3′ external duplex sequence comprises fully synthetic or partially synthetic nucleotides. In certain embodiments, the 3′ external duplex sequence is about 6 to about 50 nucleotides. In certain embodiments, the 5′ external duplex sequence is about 6 to about 50 nucleotides. In certain embodiments, the 5′ enhanced exon element further comprises a 5′ internal duplex sequence; wherein the 3′ enhanced exon element further comprises a 3′ internal duplex sequence. In certain embodiments, the 5′ internal duplex sequence and 3′ internal duplex sequence are fully complementary to each other. In certain embodiments, the 5′ internal duplex sequence and 3′ internal duplex sequence are partially complementary to each other. In certain embodiments, the 5′ internal duplex sequence and 3′ internal duplex sequences form a double-stranded duplex structure comprising at least one mismatched nucleotide pair. In certain embodiments, the double-stranded duplex structure comprises at least two mismatched nucleotide pairs. In certain embodiments, the double-stranded duplex structure comprises at least three mismatched nucleotide pairs. In certain embodiments, the double-stranded duplex structure comprises at least four mismatched nucleotide pairs. In certain embodiments, the double-stranded duplex structure comprises at least five mismatched nucleotide pairs. In certain embodiments, the 5′ internal duplex sequence comprises fully synthetic nucleotides. In certain embodiments, the 5′ internal duplex sequence comprises partially synthetic nucleotides. In certain embodiments, the 3′ internal duplex sequence comprises fully synthetic nucleotides. In certain embodiments, the 3′ internal duplex sequence comprises partially synthetic nucleotides. In certain embodiments, the 3′ internal duplex sequence is about 6 to about 19 nucleotides. In certain embodiments, the 5′ internal duplex sequence is about 6 to about 19 nucleotides. In certain embodiments, the 3′ enhanced intron segment comprises in the following order: (a) a 5′ intron segment including the second nucleotide of a 5′ group I intron splice site dinucleotide; (b) a 3′ external spacer sequence; and (c) a 3′ affinity sequence. In certain embodiments, the 3′ enhanced exon segment comprises in the following order: (a) a 5′ intron segment including the first and second nucleotide of a 5′ group I intron splice site dinucleotide; (b) a 3′ external spacer sequence; and (c) a 3′ affinity sequence; wherein the 3′ enhanced exon element comprises a 5′ exon segment lacking the first nucleotide of a 5′ group I intron splice site dinucleotide.

In certain embodiments, the precursor RNA polynucleotide comprises: (a) a leading untranslated sequence; (b) a 5′ affinity sequence; (c) 5′ external duplex sequence; (d) 5′ spacer sequence; (e) 3′ intron segment; (f) 3′ exon segment; (g) 5′ internal duplex sequence; (h) 5′ internal spacer sequence; (i) a translation initiation element; (j) a coding sequence; (k) a termination sequence; (1) a 3′ internal spacer sequence; (m) a 3′ internal duplex sequence; (n) a 5′ exon segment; (o) a 5′ intron segment; (p) a 3′ external duplex sequence; (q) a 3′ affinity sequence; and (r) a trailing untranslated sequence. In certain embodiments, (a)-(r) are arranged in the order from (a) to (r). In certain embodiments, the precursor RNA polynucleotide comprises; (a) a leading untranslated sequence; (b) a 5′ affinity sequence; (c) a 5′ external spacer sequence; (d) a 3′ intron segment; (e) a 3′ exon segment; (f) a 5′ internal duplex sequence; (g) a 5′ internal spacer sequence; (h) a translation initiation element; (i) a coding sequence; (j) a termination sequence; (k) a 3′ internal spacer sequence; (1) a 3′ internal duplex sequence; (m) a 5′ exon segment; (n) a 5′ intron segment; (o) a 3′ external spacer sequence; (p) a 3′ affinity sequence; and (q) a trailing untranslated sequence. In certain embodiments, (a)-(q) are arranged in the order from (a) to (q). In certain embodiments, the precursor RNA polynucleotide comprises: (a) a leading untranslated sequence; (b) a 5′ affinity sequence; (c) a 5′ external spacer sequence; (d) a 3′ intron segment; (e) a 3′ exon segment; (f) a 5′ internal spacer sequence; (g) a translation initiation element; (h) a coding sequence; (i) a termination sequence; (j) a 3′ internal spacer sequence; (k) a 5′ exon segment; (1) a 5′ intron segment; (m) a 3′ external spacer sequence; (n) a 3′ affinity sequence; and (o) a trailing untranslated sequence. In certain embodiments, (a)-(o) are arranged in the order from (a) to (o). In certain embodiments, the precursor RNA polynucleotide comprises: (a) a leading untranslated sequence; (b) a 5′ affinity sequence; (c) 5′ external duplex sequence; (d) 5′ spacer sequence; (e) 3′ intron segment; (f) 3′ exon segment; (g) 5′ internal duplex sequence (h) 5′ internal spacer sequence; (i) a termination sequence; (j) a coding sequence; (k) a translation initiation element; (1) a 3′ internal spacer sequence; (m) a 3′ internal duplex sequence; (n) a 5′ exon segment; (o) a 5′ intron segment; (p) a 3′ external duplex sequence; (q) a 3′ affinity sequence; and (r) a trailing untranslated sequence. In certain embodiments, (a)-(r) are arranged in the order from (a) to (r).

In certain embodiments, the coding sequence comprises two or more protein coding regions. In certain embodiments, the coding sequence comprises a sequence encoding a proteolytic cleavage site and/or a ribosomal stuttering element between the first and second expression sequence. In certain embodiments, the ribosomal stuttering element is a self-cleaving spacer. In certain embodiments, the precursor RNA polynucleotide further comprises a polynucleotide sequence encoding 2A ribosomal stuttering peptide. In certain embodiments, the precursor RNA polynucleotide comprises the following sequences operably linked to the IRES and/or operable linked to one another: (1) a 3′ group I intron segment; (2) a coding sequence that encodes the therapeutic protein; and (3) a 5′ group I intron segment. In certain embodiments, the 3′ group I intron segment and the 5′ group I intron segment are each derived from a bacterial phage, a viral vector, an organelle genome, or a nuclear rDNA gene. In certain embodiments, the 3′ group I intron segment and the 5′ group I intron segment are each derived from an anabaena bacterium, a T4 phage virus, a twort bacteriophage, a tetrahymena, or an azoarcus bacterium. In certain embodiments, the precursor RNA polynucleotide comprises one or more spacer sequences, said one or more spacer sequences being operably connected to at least one of the 3′ group I intron segment, IRES sequence, coding sequence, and 5′ group I intron segment. In certain embodiments, the precursor RNA polynucleotide comprises two spacer sequences. In certain embodiments, the two spacer sequences comprise a 5′ external spacer sequence and a 3′ external spacer sequence, or a 5′ internal spacer sequence and a 3′ internal spacer sequence. In certain embodiments, the precursor RNA polynucleotide comprises four spacer sequences. In certain embodiments, the four spacer sequences comprise a 5′ external spacer sequence, a 3′ external spacer sequence, a 5′ internal spacer sequence, and a 3′ internal spacer sequence. In certain embodiments, the precursor RNA polynucleotide comprises a 3′ exon segment and a 5′ exon segment, each derived from a natural exon. In certain embodiments, the precursor RNA polynucleotide comprises the following elements operably linked to one another: (a) the 5′ external spacer sequence; (b) the 3′ group I intron segment; (c) the 5′ exon segment; (d) the 5′ internal duplex sequence; (e) the IRES sequence; (f) the coding sequence; (g) the 3′ internal duplex sequence; (h) the 3′ exon segment; (j) the 5′ group I intron segment; and (k) the 3′ external spacer sequence. In certain embodiments, elements (a)-(k) are arranged in the order of (a)-(k). In certain embodiments, the precursor RNA polynucleotide comprises the following elements operably linked to one another: (a) the 3′ group I intron segment; (b) the 5′ exon segment; (c) the 5′ internal duplex sequence; (d) the 5′ internal spacer sequence; (e) the IRES sequence; (f) the coding sequence; (g) the 3′ internal spacer sequence; (h) the 3′ internal duplex sequence; (i) the 3′ exon segment; and (j) the 5′ group I intron segment. In certain embodiments, elements (a)-(j) are arranged in the order of (a)-(j). In certain embodiments, the precursor RNA polynucleotide comprises the following elements operably linked to one another: (a) the 5′ external spacer sequence; (b) the 3′ group I intron segment; (c) the 5′ exon segment; (d) the 5′ internal duplex sequence; (e) the 5′ internal spacer sequence; (f) the IRES sequence; (g) the coding sequence; (h) the 3′ internal spacer sequence; (i) the 3′ internal duplex sequence; (j) the 5′ exon element; (k) the 5′ group I intron segment; and (1) the 3′ external spacer sequence. In certain embodiments, elements (a)-(l) are arranged in the order of (a)-(l). In certain embodiments, the precursor RNA polynucleotide comprises fully synthetic nucleotides. In certain embodiments, the precursor RNA polynucleotide comprises partially synthetic nucleotides. In certain embodiments, the precursor RNA polynucleotide is transcribed from a vector or DNA polynucleotide comprising a PCR product, a linearized plasmid, a non-linearized plasmid, a linearized minicircle, a non-linearized minicircle, a viral vector, a cosmid, a cDNA, or an artificial chromosome.

Disclosed herein, in certain embodiments, is an oRNA produced using the precursor RNA polynucleotide of any one of the foregoing aspects and embodiments. In certain embodiments, the oRNA comprises the IRES sequence and the coding sequence. In certain embodiments, the IRES sequence is upstream of the coding sequence. In certain embodiments, the IRES sequence is downstream of the coding sequence. In certain embodiments, the oRNA comprises: (a) the 5′ exon segment; (b) the 5′ internal duplex sequence; (c) the 5′ internal spacer sequence; (d) the IRES sequence; (e) the coding sequence; (f) the 3′ internal spacer sequence (g) the 3′ internal duplex sequence; and (h) the 5′ exon element. In certain embodiments, (a)-(h) are arranged in the order from (a) to (h).

Disclosed herein, in certain embodiments, is a pharmaceutical composition comprising an oRNA comprising a sequence having at least 85% sequence identity to a sequence set forth in any one of SEQ ID NOS: 14067-24829, and a pharmaceutically acceptable salt, buffer, diluent, or combination thereof.

Disclosed herein, in certain embodiments, is a pharmaceutical composition comprising an oRNA comprising a sequence having at least 85% sequence identity to a sequence set forth in any one of SEQ ID NOS: 14067-24829, and a pharmaceutically acceptable salt, buffer, diluent, or combination thereof, wherein the oRNA is capable of expressing a therapeutic protein in a cell.

Disclosed herein, in certain embodiments, is a pharmaceutical composition comprising an oRNA comprising a sequence having at least 85% sequence identity to a sequence set forth in any one of SEQ ID NOS: 14067-24829, a cell, and a pharmaceutically acceptable salt, buffer, diluent, or combination thereof, wherein the oRNA is capable of expressing a therapeutic protein in the cell.

Disclosed herein, in certain embodiments, is a pharmaceutical composition comprising an oRNA comprising a sequence having at least 85% sequence identity to a sequence set forth in any one of SEQ ID NOS: 14067-24829, a transfer vehicle capable of delivering the oRNA to a cell, and a pharmaceutically acceptable salt, buffer, diluent, or combination thereof, wherein the oRNA is capable of expressing a therapeutic protein in a cell.

Disclosed herein, in certain embodiments, is a pharmaceutical composition comprising an oRNA comprising a sequence having at least 85% sequence identity to a sequence set forth in any one of SEQ ID NO: 793, 876, 1017, 1216, and 3291, and a pharmaceutically acceptable salt, buffer, diluent, or combination thereof, wherein the oRNA is capable of expressing a therapeutic protein in a T cell.

Disclosed herein, in certain embodiments, is a pharmaceutical composition comprising an oRNA comprising a sequence having at least 85% sequence identity to a sequence set forth in any one of SEQ ID NO: 793, 876, 1017, 1216, and 3291, a T cell, and a pharmaceutically acceptable salt, buffer, diluent, or combination thereof.

Disclosed herein, in certain embodiments, is a pharmaceutical composition comprising an oRNA comprising a sequence having at least 85% sequence identity to a sequence set forth in any one of SEQ ID NO: 793, 876, 1017, 1216, and 3291, a transfer vehicle capable of delivering the oRNA into a T cell, and a pharmaceutically acceptable salt, buffer, diluent, or combination thereof.

In certain embodiments, pharmaceutical composition comprising an oRNA comprising a sequence having at least 85% sequence identity to a sequence set forth in any one of SEQ ID NO: 793, 876, 1017, 1216, and 3291, a transfer vehicle capable of delivering the oRNA into a T cell, and a pharmaceutically acceptable salt, buffer, diluent, or combination thereof. In certain embodiments, the sequence has at least 95% sequence identity to a sequence set forth in any one of SEQ ID NOs: 793, 876, 1017, 1216, and 3291. In certain embodiments, the sequence has at least 98% sequence identity to a sequence set forth in any one of SEQ ID NOs: 793, 876, 1017, 1216, and 3291. In certain embodiments, the sequence has at least 98% sequence identity to a sequence set forth in any one of SEQ ID NOs: 793, 876, 1017, 1216, and 3291. In certain embodiments, the sequence comprise a sequence set forth in any one of SEQ ID NOs: 793, 876, 1017, 1216, and 3291.

Disclosed herein, in certain embodiments, is a pharmaceutical composition comprising an oRNA comprising an sequence having at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, and a pharmaceutically acceptable salt, buffer, diluent, or combination thereof, wherein the oRNA is capable of expressing a therapeutic protein in an immune cell, e.g., a T cell, a myeloid cell, and/or an NK cell.

Disclosed herein, in certain embodiments, is a pharmaceutical composition comprising an oRNA comprising a sequence having at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, a T cell, and a pharmaceutically acceptable salt, buffer, diluent, or combination thereof.

Disclosed herein, in certain embodiments, is a pharmaceutical composition comprising an oRNA comprising a sequence having at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, a transfer vehicle capable of delivering the oRNA into a T cell, and a pharmaceutically acceptable salt, buffer, diluent, or combination thereof.

In certain embodiments, the TIE comprises a sequence that has at least 90% identity to a sequence set forth in any one of SEQ ID NOS: 14067-24829. In certain embodiments, the sequence has at least 95% identity to a sequence set forth in any one of SEQ ID NOS: 14067-24829. In certain embodiments, the sequence has at least 98% identity to a sequence set forth in any one of SEQ ID NOS: 14067-24829. In certain embodiments, the sequence has at least 99% identity to a sequence set forth in any one of SEQ ID NOS: 14067-24829. In certain embodiments, the sequence comprises a sequence set forth in any one of SEQ ID NOS: 14067-24829.

In certain embodiments, the TIE comprises a sequence that has at least 90% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the sequence has at least 95% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the sequence has at least 98% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the sequence has at least 99% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In certain embodiments, the sequence comprises a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

Disclosed herein, in certain embodiments, is a pharmaceutical composition comprising an oRNA comprising a sequence having at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, and a pharmaceutically acceptable salt, buffer, diluent, or combination thereof, wherein the oRNA is capable of expressing a therapeutic protein in a T cell.

Disclosed herein, in certain embodiments, is a pharmaceutical composition comprising an oRNA comprising a sequence having at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, a T cell, and a pharmaceutically acceptable salt, buffer, diluent, or combination thereof.

Disclosed herein, in certain embodiments, is a pharmaceutical composition comprising an oRNA comprising an sequence having at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, a transfer vehicle capable of delivering the oRNA into a T cell, and a pharmaceutically acceptable salt, buffer, diluent, or combination thereof.

In certain embodiments, the TIE comprises a sequence that has at least 90% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the sequence has at least 95% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the sequence has at least 98% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the sequence has at least 99% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In certain embodiments, the sequence comprise a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

In certain embodiments, the oRNA comprises the following elements, in the following order: (1) the TIE sequence, e.g., comprising an IRES sequence; and (2) a coding sequence encoding a therapeutic protein, wherein elements (1) and (2) are operably linked to one another. In certain embodiments, the TIE sequence, e.g., comprising an IRES sequence is capable of facilitating expression of the therapeutic protein encoded by a precursor RNA polynucleotide in the cell. In certain embodiments, the TIE sequence, e.g., comprising an IRES sequence, is capable of facilitating expression of the therapeutic protein in the cell, such that the expression level of the protein in the cell is comparable to or higher than when a control TIE sequence, e.g., comprising an IRES sequence, is used (e.g., SEQ ID NO: 3303). In certain embodiments, the TIE sequence, e.g., comprising an IRES sequence, is capable of facilitating expression of the therapeutic protein in the cell, such that the expression level of the protein in the cell is higher than when a control TIE sequence, e.g., comprising an IRES sequence, is used (e.g., SEQ ID NO: 3303) by about 1.5-fold, 2-fold, 3-fold, 4-fold, 5-fold, or higher as compared to the expression mediated by the control TIE sequence, e.g., comprising an IRES sequence (e.g., SEQ ID NO: 3303).

In certain embodiments, the TIE sequence, e.g., comprising an IRES sequence, is capable of facilitating expression of the therapeutic protein encoded by a precursor RNA polynucleotide in an immune cell. In certain embodiments, the TIE sequence, e.g., comprising an IRES sequence, is capable of facilitating expression of the therapeutic protein in the immune cell, such that the expression level of the protein in the immune cell is comparable to or higher than when a control TIE sequence, e.g., comprising an IRES sequence, is used (e.g., SEQ ID NO: 3303). In certain embodiments, the TIE sequence, e.g., comprising an IRES sequence, is capable of facilitating expression of the therapeutic protein in the immune cell, such that the expression level of the protein in the immune cell is higher than when a control TIE sequence, e.g., comprising an IRES sequence, is used (e.g., SEQ ID NO: 3303) by about 1.5-fold, 2-fold, 3-fold, 4-fold, 5-fold, or higher as compared to the expression mediated by the control TIE sequence, e.g., comprising an IRES sequence (e.g., SEQ ID NO: 3303). Immune cells include, but are not limited to, T cells, myeloid cells (e.g., macrophages), and NK cells.

In certain embodiments, the TIE sequence, e.g., comprising an IRES sequence, is capable of facilitating expression of the therapeutic protein encoded by a precursor RNA polynucleotide in a non-immune cell, e.g., a muscle or liver cell. In certain embodiments, the TIE sequence, e.g., comprising an IRES sequence, is capable of facilitating expression of the therapeutic protein in the non-immune cell, e.g., a muscle or liver cell, such that the expression level of the protein in the non-immune cell, e.g., a muscle or liver cell, is comparable to or higher than when a control TIE sequence, e.g., comprising an IRES sequence, is used (e.g., SEQ ID NO: 3303). In certain embodiments, the TIE sequence, e.g., comprising an IRES sequence, is capable of facilitating expression of the therapeutic protein in the non-immune cell, e.g., a muscle or liver cell, such that the expression level of the protein in the non-immune cell, e.g., a muscle or liver cell, is higher than when a control TIE sequence, e.g., comprising an IRES sequence, is used (e.g., SEQ ID NO: 3303) by about 1.5-fold, 2-fold, 3-fold, 4-fold, 5-fold, or higher as compared to the expression mediated by the control TIE sequence, e.g., comprising an IRES sequence (e.g., SEQ ID NO: 3303).

In certain embodiments, the TIE sequence, e.g., comprising an IRES sequence, is capable of facilitating expression of the therapeutic protein encoded by a precursor RNA polynucleotide in the T cell. In certain embodiments, the TIE sequence, e.g., comprising an IRES sequence, is capable of facilitating expression of the therapeutic protein in the T cell, such that the expression level of the protein in the T cell is comparable to or higher than when a control IRES is used (e.g., SEQ ID NO: 3303). In certain embodiments, the TIE sequence, e.g., comprising an IRES sequence, is capable of facilitating expression of the therapeutic protein in the T cell, such that the expression level of the protein in the T cell is higher than when a control TIE sequence, e.g., comprising an IRES sequence, is used (e.g., SEQ ID NO: 3303) by about 1.5-fold, 2-fold, 3-fold, 4-fold, 5-fold, or higher as compared to the expression mediated by the control TIE sequence, e.g., comprising an IRES sequence (e.g., SEQ ID NO: 3303).

In certain embodiments, the therapeutic protein comprises a chimeric protein. In certain embodiments, the chimeric protein comprises a chimeric antigen receptor (CAR), a T-cell receptor (TCR), a B-cell receptor (BCR), an immune cell activation or inhibitory receptor, a recombinant fusion protein, a chimeric mutant protein, or a fusion protein, or a combination thereof. In certain embodiments, the therapeutic protein comprises an antibody, a nanobody, a non-antibody protein, an immune modulatory ligand, a receptor, a structural protein, a growth factor ligand or receptor, a hormone or hormone receptor, a transcription factor, a checkpoint inhibitor or agonist, a Fc fusion protein, an anticoagulant, a blood clotting factor, a chaperone protein, a antimicrobial protein, a structural protein, a biochemical enzyme, a tight junction protein, a mitochondrial stress response, a cytoskeletal protein, a metal-binding protein, or a small molecule, or combinations thereof. In certain embodiments, the therapeutic protein comprises an antibody, a nanobody, a non-antibody protein, an immune modulatory ligand, a receptor, a structural protein, a growth factor ligand or receptor, a hormone or hormone receptor, a transcription factor, a checkpoint inhibitor or agonist, a Fc fusion protein, an anticoagulant, a blood clotting factor, a chaperone protein, a antimicrobial protein, a structural protein, a biochemical enzyme, a tight junction protein, a mitochondrial stress response, a cytoskeletal protein, a metal-binding protein, or a small molecule, or combinations thereof. In certain embodiments, the structural protein comprises a channel protein or nuclear pore protein. In certain embodiments, the coding sequence is codon-optimized. In certain embodiments, the coding sequence is codon-optimized. In certain embodiments, the coding sequence is codon-optimized. In certain embodiments, the coding sequence is optimized to have G-C content that is between 50% and 70%. In certain embodiments, the coding sequence is optimized to have G-C content that is between 55% and 64%. In certain embodiments, the oRNA is from about 0.1 to about 15 kilobases in length. In certain embodiments, the pharmaceutical composition has an in vivo duration of therapeutic effect in humans of at least 20 hours. In certain embodiments, the pharmaceutical composition has a functional half-life of at least 6 hours. In certain embodiments, the pharmaceutical composition has a duration of therapeutic effect in a human cell greater than or equal to that of an equivalent linear RNA polynucleotide comprising the same expression sequence. In certain embodiments, the pharmaceutical composition has an in vivo duration of therapeutic effect in human greater than that of an equivalent linear RNA polynucleotide having the same expression sequence. In certain embodiments, an in vivo duration of therapeutic effect in human greater than that of an equivalent linear RNA polynucleotide having the same expression sequence. In certain embodiments, the pharmaceutical composition is formulated for delivery to a T cell via electroporation. In certain embodiments, the oRNA is comprised in a nucleic acid expression vector. In certain embodiments, the nucleic acid expression vector is selected from the group consisting of a PCR product, a linearized plasmid, a non-linearized plasmid, a linearized minicircle, a non-linearized minicircle, a cosmid, a cDNA, or an artificial chromosome.

In certain embodiments, the transfer vehicle comprises a nanoparticle. In certain embodiments, the nanoparticle is a lipid nanoparticle, a core-shell nanoparticle, a biodegradable nanoparticle, a biodegradable lipid nanoparticle, a polymer nanoparticle, a polyplex or a biodegradable polymer nanoparticle. In certain embodiments, the nanoparticle is a lipid nanoparticle, a core-shell nanoparticle, or a biodegradable nanoparticle. In certain embodiments, the nanoparticle comprises one or more cationic lipids, ionizable lipids, or poly β-amino esters, or combinations thereof. In certain embodiments, the nanoparticle comprises one or more non-cationic lipids. In certain embodiments, the nanoparticle comprises one or more non-cationic lipids. In certain embodiments, the one or more structural lipids comprise cholesterol. In certain embodiments, the nanoparticle comprises arachidonic acid, leukotriene, oleic acid, or combinations thereof. In certain embodiments, the molar ratio of the ionizable lipid in the transfer vehicle is from about 40 to about 60% of the total lipid present in the transfer vehicle. In certain embodiments, the molar ratio of the helper lipid in the transfer vehicle is from about 3.5% to about 14% of the total lipid present in the transfer vehicle. In certain embodiments, the molar ratio of the PEG-lipid in the transfer vehicle is from about 0.5% to about 5% of the total lipid present in the LNP. In certain embodiments, the structural lipid in the transfer vehicle is from about 28% to about 50% of the total lipid present in the transfer vehicle. In certain embodiments, the molar ratio of ionizable lipid:helper lipid:structural lipid:PEG-lipid is about 45:9:44:2, about 50:10:38.5:1.5, about 41:12:45:2, about 62:4:33:1, or about 53:5:41:1. In certain embodiments, the nanoparticle has a lipid to phosphate (IL:P) ratio of about 3 to about 6, such as about 3, about 4, about 4.5, about 5, about 5.5, or about 6. In certain embodiments, the transfer vehicle is formulated for endosomal release of the circular RNA polynucleotide. In certain embodiments, the nanoparticle comprises a targeting moiety operably connected thereto, wherein the targeting moiety mediates receptor-mediated endocytosis, endosome fusion, or direct fusion into cells in the absence of cell isolation or purification. In certain embodiments, the targeting moiety comprises a small molecule, a scFv, a nanobody, a peptide, a cyclic peptide, a di or tri cyclic peptide, minibody, a polynucleotide an aptamer, an engineered a scaffold protein, a heavy chain variable region, a light chain variable region, or a fragment thereof. In certain embodiments, the transfer vehicle comprises a liposome, a dendrimer, a carbohydrate carrier, glycan nanomaterial, fusome, exosome, or a combination thereof.

In certain embodiments, the nanoparticle comprises a targeting moiety operably connected thereto, wherein the targeting moiety mediates receptor-mediated endocytosis, endosome fusion, or direct fusion into T cells in the absence of cell isolation or purification. In certain embodiments, the T cell is a CD8+ cytotoxic T cell, a CD4+ helper T cell (Th), a regulatory T (Treg) cell, a memory T cell, or an innate-like T cell. In certain embodiments, the Th cell is a Th1 cell, a Th2 cell, a Th17 cell, a Th9 cell, a Tfh cell, or a Th22 cell. In certain embodiments, the memory T cell is a central memory T cell (Tcm), an effector memory T cell (Tem), a tissue-resident memory T cell (Trm), or a virtual memory T cell. In certain embodiments, the innate-like T cell is a natural killer T (NKT) cell, a mucosal-associated invariant T cell (MAIT), or a gamma delta T cell (γδ T cell).

Disclosed herein, in certain embodiments, is a eukaryotic cell comprising the precursor RNA polynucleotide, oRNA, or pharmaceutical composition of any of the foregoing aspects and embodiments. In some embodiments, the eukaryotic cell is a human cell. In certain embodiments, the eukaryotic cell is an immune cell. In certain embodiments, the eukaryotic cell is a T cell, dendritic cell, macrophage, B cell, neutrophil, or basophil.

Disclosed herein, in certain embodiments, is a T cell comprising precursor RNA polynucleotide, oRNA, or pharmaceutical composition of any of the foregoing aspects and embodiments. In certain embodiments, the T cell is a human T cell. In certain embodiments, the T cell is a CD8+ cytotoxic T cell, a CD4+ helper T cell (Th), a regulatory T (Treg) cell, a memory T cell, an innate-like T cell. In certain embodiments, the Th cell is a Th1 cell, a Th2 cell, a Th17 cell, a Th9 cell, a Tfh cell, or a Th22 cell. In certain embodiments, the memory T cell is a central memory T cell (Tcm), an effector memory T cell (Tem), a tissue-resident memory T cell (Trm), or a virtual memory T cell. In certain embodiments, the innate-like T cell is a natural killer T (NKT) cell, a mucosal-associated invariant T cell (MAIT), or a gamma delta T cell (γδ T cell).

Disclosed herein, in certain embodiments, is a prokaryotic cell comprising the precursor RNA polynucleotide, oRNA, or pharmaceutical composition of any of the foregoing aspects and embodiments.

Disclosed herein, in certain embodiments, is a method of expressing a therapeutic protein in a cell, comprising contacting the cell with the precursor RNA polynucleotide, oRNA, or pharmaceutical composition of any of the foregoing aspects and embodiments, thereby expressing the therapeutic protein in the cell.

Disclosed herein, in certain embodiments, is a method of expressing a therapeutic protein in a T cell, comprising contacting the T cell with the precursor RNA polynucleotide, oRNA, or pharmaceutical composition of any of the foregoing aspects and embodiments, thereby expressing the therapeutic protein in the T cell. In certain embodiments, the TIE sequence, e.g., comprising an IRES sequence, is capable of facilitating expression of the therapeutic protein in the T cell, such that the expression level of the protein in the T cell is comparable to or higher than when a control TIE sequence, e.g., comprising an IRES sequence, is used (e.g., SEQ ID NO: 3303). In certain embodiments, the TIE sequence, e.g., comprising an IRES sequence, is capable of facilitating expression of the therapeutic protein in the T cell, such that the expression level of the protein in the T cell is higher than when a control TIE sequence, e.g., comprising an IRES sequence, is used (e.g., SEQ ID NO: 3303) by about 1.5-fold, 2-fold, 3-fold, 4-fold, 5-fold, or higher as compared to the expression mediated by the control IRES (e.g., SEQ ID NO: 3303).

Disclosed herein, in certain embodiments, is a method of expressing a protein from an oRNA molecule in a cell, comprising providing the cell with an oRNA comprising an IRES selected from a sequence set forth in any one SEQ ID NOS: 14067-24829, adjacent to a coding sequence.

Disclosed herein, in certain embodiments, is a method of expressing a protein from an oRNA molecule in a T cell, comprising providing the T cell with an oRNA comprising an IRES selected from a sequence set forth in any one of SEQ ID NOs: 793, 876, 1017, 1216, and 3291 adjacent to a coding sequence. In certain embodiments, the IRES is capable of facilitating expression of the therapeutic protein in the T cell, such that the expression level of the protein in the T cell is comparable to or higher than when a control IRES is used (e.g., SEQ ID NO: 3303). In certain embodiments, the IRES is capable of facilitating expression of the therapeutic protein in the T cell, such that the expression level of the protein in the T cell is higher than when a control IRES is used (e.g., SEQ ID NO: 3303) by about 1.5-fold, 2-fold, 3-fold, 4-fold, 5-fold, or higher as compared to the expression mediated by the control IRES (e.g., SEQ ID NO: 3303).

Disclosed herein, in certain embodiments, is a method of expressing a protein from an oRNA molecule in a T cell, comprising providing the T cell with an oRNA comprising an IRES selected from a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302 adjacent to a coding sequence. In certain embodiments, the IRES is capable of facilitating expression of the therapeutic protein in the T cell, such that the expression level of the protein in the T cell is comparable to or higher than when a control IRES is used (e.g., SEQ ID NO: 3303). In certain embodiments, the IRES is capable of facilitating expression of the therapeutic protein in the T cell, such that the expression level of the protein in the T cell is higher than when a control IRES is used (e.g., SEQ ID NO: 3303) by about 1.5-fold, 2-fold, 3-fold, 4-fold, 5-fold, or higher as compared to the expression mediated by the control IRES (e.g., SEQ ID NO: 3303).

Disclosed herein, in certain embodiments, is a method of expressing a protein from an oRNA molecule in a T cell, comprising providing the T cell with an oRNA comprising an IRES selected from a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301 adjacent to a coding sequence. In certain embodiments, the IRES is capable of facilitating expression of the therapeutic protein in the T cell, such that the expression level of the protein in the T cell is comparable to or higher than when a control IRES is used (e.g., SEQ ID NO: 3303). In certain embodiments, the IRES is capable of facilitating expression of the therapeutic protein in the T cell, such that the expression level of the protein in the T cell is higher than when a control IRES is used (e.g., SEQ ID NO: 3303) by about 1.5-fold, 2-fold, 3-fold, 4-fold, 5-fold, or higher as compared to the expression mediated by the control IRES (e.g., SEQ ID NO: 3303).

Disclosed herein, in certain embodiments, is a method of treating a subject having a disease or disorder, the method comprising administering the pharmaceutical composition of any of the foregoing aspects and embodiments. In certain embodiments, the disease or disorder is a cancer. In certain embodiments, the disease or disorder is an autoimmune disease or disorder. In certain embodiments, the subject is a human.

### BRIEF DESCRIPTION OF THE DRAWINGS

FIG. 1 depicts luminescence in supernatants of HEK293 (FIGS. 1A, 1D, and 1E), HepG2 (FIG. 1B), or 1C1C7 (FIG. 1C) cells 24 hours after transfection with circular RNA comprising a Gaussia luciferase expression sequence and various IRES sequences.

FIG. 2 depicts luminescence in supernatants of HEK293 (FIG. 2A), HepG2 (FIG. 2B), or 1C1C7 (FIG. 2C) cells 24 hours after transfection with circular RNA comprising a Gaussia luciferase expression sequence and various IRES sequences having different lengths.

FIG. 3 depicts stability of select IRES constructs in HepG2 (FIG. 3A) or 1C1C7 (FIG. 3B) cells over 3 days as measured by luminescence.

FIGS. 4A and 4B depict protein expression from select IRES constructs in Jurkat cells, as measured by luminescence from secreted Gaussia luciferase in cell supernatants.

FIGS. 5A and 5B depict stability of select IRES constructs in Jurkat cells over 3 days as measured by luminescence.

FIG. 6 depicts comparisons of 24 hour luminescence (FIG. 6A) or relative luminescence over 3 days (FIG. 6B) of modified linear, unpurified circular, or purified circular RNA encoding Gaussia luciferase.

FIG. 7 depicts transcript induction of IFNγ (FIG. 7A), IL-6 (FIG. 7B), IL-2 (FIG. 7C), RIG-I (FIG. 7D), IFN-β1 (FIG. 7E), and TNFα (FIG. 7F) after electroporation of Jurkat cells with modified linear, unpurified circular, or purified circular RNA.

FIG. 8 depicts a comparison of luminescence of circular RNA and modified linear RNA encoding Gaussia luciferase in human primary monocytes (FIG. 8A) and macrophages (FIG. 8B and FIG. 8C).

FIG. 9 depicts relative luminescence over 3 days (FIG. 9A) in supernatant of primary T cells after transduction with circular RNA comprising a Gaussia luciferase expression sequence and varying IRES sequences or 24 hour luminescence (FIG. 9B).

FIG. 10 depicts 24 hour luminescence in supernatant of primary T cells (FIG. 10A) after transduction with circular RNA or modified linear RNA comprising a Gaussia luciferase expression sequence, or relative luminescence over 3 days (FIG. 10B), and 24 hour luminescence in PBMCs (FIG. 10C).

FIG. 11 depicts HPLC chromatograms (FIG. 11A) and circularization efficiencies (FIG. 11B) of RNA constructs having different permutation sites.

FIG. 12 depicts HPLC chromatograms (FIG. 12A) and circularization efficiencies (FIG. 12B) of RNA constructs having different introns and/or permutation sites.

FIG. 13 depicts HPLC chromatograms (FIG. 13A) and circularization efficiencies (FIG. 13B) of 3 RNA constructs with or without homology arms.

FIG. 14 depicts circularization efficiencies of 3 RNA constructs without homology arms or with homology arms having various lengths and GC content.

FIGS. 15A and 15B depict HPLC chromatograms showing the contribution of strong homology arms to improved splicing efficiency, the relationship between circularization efficiency and nicking in select constructs, and combinations of permutations sites and homology arms hypothesized to demonstrate improved circularization efficiency.

FIG. 16 shows fluorescent images of T cells mock electroporated (left) or electroporated with circular RNA encoding a CAR (right) in co-cultured with Raji cells expressing GFP and firefly luciferase.

FIG. 17 shows bright field (left), fluorescent (center), and overlay (right) images of T cells mock electroporated (top) or electroporated with circular RNA encoding a CAR (bottom) and co-cultured with Raji cells expressing GFP and firefly luciferase.

FIG. 18 depicts specific lysis of Raji target cells by T cells mock electroporated or electroporated with circular RNA encoding different CAR sequences.

FIG. 19 depicts luminescence in supernatants of Jurkat cells (left) or resting primary human CD3+ T cells (right) 24 hours after transduction with linear or circular RNA comprising a Gaussia luciferase expression sequence and varying IRES sequences (FIG. 19A), and relative luminescence over 3 days (FIG. 19B).

FIG. 20 depicts transcript induction of IFN-β1 (FIG. 20A), RIG-I (FIG. 20B), IL-2 (FIG. 20C), IL-6 (FIG. 20D), IFNγ (FIG. 20E), and TNFα (FIG. 20F) after electroporation of human CD3+ T cells with modified linear, unpurified circular, or purified circular RNA.

FIG. 21 depicts specific lysis of Raji target cells by human primary CD3+ T cells electroporated with circRNA encoding a CAR as determined by detection of firefly luminescence (FIG. 21A), and IFNγ transcript induction 24 hours after electroporation with different quantities of circular or linear RNA encoding a CAR sequence (FIG. 21B).

FIG. 22 depicts specific lysis of target or non-target cells by human primary CD3+ T cells electroporated with circular or linear RNA encoding a CAR at different E:T ratios (FIG. 22A and FIG. 22B) as determined by detection of firefly luminescence.

FIG. 23 depicts specific lysis of target cells by human CD3+ T cells electroporated with RNA encoding a CAR at 1, 3, 5, and 7 days post electroporation.

FIG. 24 depicts specific lysis of target cells by human CD3+ T cells electroporated with circular RNA encoding a CD19 or BCMA targeted CAR.

FIG. 25 depicts total Flux of organs harvested from CD-1 mice dosed with circular RNA encoding FLuc and formulated with 50% Lipid 10b-15, 10% DSPC, 1.5% PEG-DMG, and 38.5% cholesterol.

FIG. 26 shows images highlighting the luminescence of organs harvested from CD-1 mice dosed with circular RNA encoding FLuc and formulated with 50% Lipid 10b-15, 10% DSPC, 1.5% PEG-DMG, and 38.5% cholesterol.

FIG. 27 depicts molecular characterization of Lipids 10a-26 and 10a-27. FIG. 27A shows the proton nuclear magnetic resonance (NMR) spectrum of Lipid 10a-26. FIG. 27B shows the retention time of Lipid 10a-26 measured by liquid chromatography-mass spectrometry (LC-MS). FIG. 27C shows the mass spectrum of Lipid 10a-26. FIG. 27D shows the proton NMR spectrum of Lipid 10a-27. FIG. 27E shows the retention time of Lipid 10a-27 measured by LC-MS. FIG. 27F shows the mass spectrum of Lipid 10a-27.

FIG. 28 depicts molecular characterization of Lipid 22-S14 and its synthetic intermediates. FIG. 28A depicts the NMR spectrum of 2-(tetradecylthio)ethan-1-ol. FIG. 28B depicts the NMR spectrum of 2-(tetradecylthio)ethyl acrylate. FIG. 28C depicts the NMR spectrum of bis(2-(tetradecylthio)ethyl) 3,3′-((3-(2-methyl-1H-imidazol-1-yl)propyl)azanediyl)dipropionate (Lipid 22-S14).

FIG. 29 depicts the NMR spectrum of bis(2-(tetradecylthio)ethyl) 3,3′-((3-(1H-imidazol-1-yl)propyl)azanediyl)dipropionate (Lipid 93-S14).

FIG. 30 depicts molecular characterization of heptadecan-9-yl 8-((3-(2-methyl-1H-imidazol-1-yl)propyl)(8-(nonyloxy)-8-oxooctyl)amino)octanoate (Lipid 10a-54). FIG. 30A shows the proton NMR spectrum of Lipid 10a-54. FIG. 30B shows the retention time of Lipid 10a-54 measured by LC-MS. FIG. 30C shows the mass spectrum of Lipid 10a-54.

FIG. 31 depicts molecular characterization of heptadecan-9-yl 8-((3-(1H-imidazol-1-yl)propyl)(8-(nonyloxy)-8-oxooctyl)amino)octanoate (Lipid 10a-53). FIG. 31A shows the proton NMR spectrum of Lipid 10a-53. FIG. 31B shows the retention time of Lipid 10a-53 measured by LC-MS. FIG. 31C shows the mass spectrum of Lipid 10a-53.

FIG. 32A depicts total flux of spleen and liver harvested from CD-1 mice dosed with circular RNA encoding firefly luciferase (FLuc) and formulated with ionizable lipid of interest, DSPC, cholesterol, and DSPE-PEG 2000 (Avanti Polar Lipids Inc.) at a weight ratio of 16:1:4:1 or 62:4:33:1 molar ratio. FIG. 32B depicts average radiance for biodistribution of protein expression.

FIG. 33A depicts images highlighting the luminescence of organs harvested from CD-1 mice dosed with circular RNA encoding FLuc and formulated with ionizable Lipid 22-S14, DSPC, cholesterol, and DSPE-PEG 2000 (Avanti Polar Lipids Inc.) at a weight ratio of 16:1:4:1 or 62:4:33:1 molar ratio. FIG. 33B depicts whole body IVIS images of CD-1 mice dosed with circular RNA encoding FLuc and formulated with ionizable Lipid 22-S14, DSPC, cholesterol, and DSPE-PEG 2000 (Avanti Polar Lipids Inc.) at a weight ratio of 16:1:4:1 or 62:4:33:1 molar ratio.

FIG. 34A depicts images highlighting the luminescence of organs harvested from CD-1 mice dosed with circular RNA encoding FLuc and formulated with ionizable Lipid 93-S14, DSPC, cholesterol, and DSPE-PEG 2000 (Avanti Polar Lipids Inc.) at a weight ratio of 16:1:4:1 or 62:4:33:1 molar ratio. FIG. 34B depicts whole body IVIS images of CD-1 mice dosed with circular RNA encoding FLuc and formulated with ionizable Lipid 93-S14, DSPC, cholesterol, and DSPE-PEG 2000 (Avanti Polar Lipids Inc.) at a weight ratio of 16:1:4:1 or 62:4:33:1 molar ratio.

FIG. 35A depicts images highlighting the luminescence of organs harvested from CD-1 mice dosed with circular RNA encoding FLuc and formulated with ionizable Lipid 10a-26, DSPC, cholesterol, and DSPE-PEG 2000 (Avanti Polar Lipids Inc.) at a weight ratio of 16:1:4:1 or 62:4:33:1 molar ratio. FIG. 35B depicts whole body IVIS images of CD-1 mice dosed with circular RNA encoding FLuc and formulated with ionizable Lipid 10a-26, DSPC, cholesterol, and DSPE-PEG 2000 (Avanti Polar Lipids Inc.) at a weight ratio of 16:1:4:1 or 62:4:33:1 molar ratio.

FIG. 36 depicts images highlighting the luminescence of organs harvested from c57 BL/6J mice dosed with circular RNA encoding FLuc and encapsulated in lipid nanoparticles formed with Lipid 10b-15 (FIG. 36A), Lipid 10a-53 (FIG. 36B), or Lipid 10a-54 (FIG. 36C). PBS was used as control (FIG. 36D).

FIGS. 37A and 37B depict relative luminescence in the lysates of human PBMCs after 24-hour incubation with testing lipid nanoparticles containing circular RNA encoding firefly luciferase.

FIG. 38 shows the expression of GFP (FIG. 38A) and CD19 CAR (FIG. 38B) in human PBMCs after incubating with testing lipid nanoparticle containing circular RNA encoding either GFP or CD19 CAR.

FIG. 39 depicts the expression of an anti-murine CD19 CAR in 1C1C7 cells lipotransfected with circular RNA comprising an anti-murine CD19 CAR expression sequence and varying IRES sequences.

FIG. 40 shows the cytotoxicity of an anti-murine CD19 CAR to murine T cells. The CD19 CAR is encoded by and expressed from a circular RNA, which is electroporated into the murine T cells.

FIG. 41 depicts the B cell counts in peripheral blood (FIGS. 41A and 41B) or spleen (FIG. 41C) in C57BL/6J mice injected every other day with testing lipid nanoparticles encapsulating a circular RNA encoding an anti-murine CD19 CAR.

FIGS. 42A and 42B compares the expression level of an anti-human CD19 CAR expressed from a circular RNA with that expressed from a linear mRNA.

FIGS. 43A and 43B compares the cytotoxic effect of an anti-human CD19 CAR expressed from a circular RNA with that expressed from a linear mRNA

FIG. 44 depicts the cytotoxicity of two CARs (anti-human CD19 CAR and anti-human BCMA CAR) expressed from a single circular RNA in T cells.

FIG. 45A shows representative FACS plots with frequencies of tdTomato expression in various spleen immune cell subsets following treatment with LNPs formed with Lipid 10a-27 or 10a-26 or Lipid 10b-15. FIG. 45B shows the quantification of the proportion of myeloid cells, B cells, and T cells expressing tdTomato (mean+std. dev., n=3), equivalent to the proportion of each cell population successfully transfected with Cre circular RNA. FIG. 45C illustrates the proportion of additional splenic immune cell populations, including NK cells, classical monocytes, nonclassical monocytes, neutrophils, and dendritic cells, expressing tdTomato after treatment with Lipids 27 and 26 (mean+std. dev., n=3).

FIG. 46A depicts an exemplary RNA construct design with built-in polyA sequences in the introns. FIG. 46B shows the chromatography trace of unpurified circular RNA. FIG. 46C shows the chromatography trace of affinity-purified circular RNA. FIG. 46D shows the immunogenicity of the circular RNAs prepared with varying in vitro transcription (IVT) conditions and purification methods. (Commercial=commercial IVT mix; Custom=customized IVT mix; Aff=affinity purification; Enz=enzyme purification; GMP:GTP ratio=8, 12.5, or 13.75).

FIG. 47A depicts an exemplary RNA construct design with a dedicated binding sequence of TATAATTCTACCCTATTGAGGCATTGACTA (SEQ ID NO: 3269) as an alternative to polyA for hybridization purification. FIG. 47B shows the chromatography trace of unpurified circular RNA. FIG. 46C shows the chromatography trace of affinity-purified circular RNA.

FIG. 48A shows the chromatography trace of unpurified circular RNA encoding dystrophin. FIG. 48B shows the chromatography trace of enzyme-purified circular RNA encoding dystrophin.

FIG. 49 compares the expression (FIG. 49A) and stability (FIG. 49B) of purified circRNAs with different 5′ spacers between the 3′ intron segment/5′ internal duplex region and the IRES in Jurkat cells. (AC=only A and C were used in the spacer sequence; UC=only U and C were used in the spacer sequence.)

FIG. 50 shows luminescence expression levels and stability of expression in primary T cells from circular RNAs containing the original or modified IRES elements indicated.

FIG. 51 shows luminescence expression levels and stability of expression in HepG2 cells from circular RNAs containing the original or modified IRES elements indicated.

FIG. 52 shows luminescence expression levels and stability of expression in 1C1C7 cells from circular RNAs containing the original or modified IRES elements indicated.

FIG. 53 shows luminescence expression levels and stability of expression in HepG2 cells from circular RNAs containing IRES elements with untranslated regions (UTRs) inserted or hybrid IRES elements. “Scr” means Scrambled, which was used as a control.

FIG. 54 shows luminescence expression levels and stability of expression in 1C1C7 cells from circular RNAs containing an IRES and variable stop codon cassettes operably linked to a Gaussia luciferase coding sequence.

FIG. 55 shows luminescence expression levels and stability of expression in 1C1C7 cells from circular RNAs containing an IRES and variable untranslated regions (UTRs) inserted before the start codon of a Gaussia luciferase coding sequence.

FIG. 56 shows expression levels of human erythropoietin (hEPO) in Huh7 cells from circular RNAs containing two miR-122 target sites downstream from the hEPO coding sequence.

FIG. 57 shows immune cell expression of mOX40L in wildtype mice following intravenous injection of LNPs that have been transfected with circular RNAs encoding mOX40L.

FIG. 58 shows B cell depletion of LNPs transfected intravenously with circular RNAs in mice. FIG. 58A quantifies B cell depletion through B220+ B cells of live, CD45+ immune cells and FIG. 58B compares B cell depletion of B220+ B cells of live, CD45+ immune cells in comparison to luciferase expressing circular RNAs. FIG. 58C provides B cell weight gain of the transfected cells.

FIG. 59 shows CAR expression levels in the peripheral blood (FIG. 59A) and spleen (FIG. 59B) when treated with LNP encapsulating circular RNA that expresses anti-CD19 CAR. Anti-CD20 (aCD20) and circular RNA encoding luciferase (oLuc) were used for comparison.

FIG. 60 shows the overall frequency of anti-CD19 CAR expression, the frequency of anti-CD19 CAR expression on the surface of cells and effect on anti-tumor response of IRES specific circular RNA encoding anti-CD19 CARs on T cells. FIG. 60A shows anti-CD19 CAR geometric mean florescence intensity, FIG. 60B shows percentage of anti-CD19 CAR expression, and FIG. 60C shows the percentage target cell lysis performed by the anti-CD19 CAR. (CK=Caprine Kobuvirus; AP=Apodemus Picornavirus; CK*=Caprine Kobuvirus with codon optimization; PV=Parabovirus; SV=Salivirus.)

FIG. 61 shows CAR expression levels of A20 FLuc target cells when treated with IRES specific circular RNA constructs.

FIG. 62 shows luminescence expression levels for cytosolic (FIG. 62A) and surface (FIG. 62B) proteins from circular RNA in primary human T cells.

FIG. 63 shows luminescence expression in human T cells when treated with IRES specific circular constructs. Expression in circular RNA constructs were compared to linear mRNA. FIG. 63A, FIG. 63B, and FIG. 63G provide Gaussia luciferase expression in multiple donor cells. FIG. 63C, FIG. 63D, FIG. 63E, and FIG. 63F provides firefly luciferase expression in multiple donor cells.

FIG. 64 shows anti-CD19 CAR (FIG. 64A and FIG. 64B) and anti-BCMA CAR (FIG. 63B) expression in human T cells following treatment of a lipid nanoparticle encompassing a circular RNA that encodes either an anti-CD19 or anti-BCMA CAR to a firefly luciferase expressing K562 cell.

FIG. 65 shows anti-CD19 CAR expression levels resulting from delivery via electroporation in vitro of a circular RNA encoding an anti-CD19 CAR in a specific antigen-dependent manner. FIG. 65A shows Nalm6 cell lysing with an anti-CD19 CAR. FIG. 65B shows K562 cell lysing with an anti-CD19 CAR.

FIG. 66 shows transfection of LNP mediated by use of ApoE3 in solutions containing LNP and circular RNA expressing green fluorescence protein (GFP). FIG. 66A showed the live-dead results. FIG. 66B, FIG. 66C, FIG. 66D, and FIG. 66E provide the frequency of expression for multiple donors.

FIG. 67 provides multiple controlled adjuvant strategies. CircRNA as indicated on the figure entails an unpurified sense circular RNA splicing reaction using GTP as an indicator molecule in vitro. 3p-circRNA entails a purified sense circular RNA as well as a purified antisense circular RNA mixed containing triphosphorylated 5′ termini. FIG. 67A shows IFN-β Induction in vitro in wild type and MAVS knockout A549 cells and FIG. 67B shows in vivo cytokine response to formulated circRNA generated using the indicated strategy.

FIG. 68 illustrates an intramuscular delivery of LNP containing circular RNA constructs. FIG. 68A provides a live whole body flux post a 6 hour period and FIG. 68B provides whole body IVIS 6 hours following a 1 μg dose of the LNP-circular RNA construct.

FIG. 68C provides an ex vivo expression distribution over a 24-hour period.

FIG. 69 illustrates expression of multiple circular RNAs from a single lipid formulation. FIG. 69A provides hEPO titers from a single and mixed set of LNP containing circular RNA constructs, while FIG. 69B provides total flux of bioluminescence expression from single or mixed set of LNP containing circular RNA constructs.

FIG. 70 depicts a general sequence construct of a linear RNA polynucleotide precursor (10). The sequence as provided is illustrated in a 5′ to 3′ order of a 5′ enhanced intron element (20), a 5′ enhanced exon element (30), a core functional element (40), a 3′ enhanced exon element (50) and a 3′ enhanced intron element (60).

FIG. 71 depicts various exemplary iterations of the 5′ enhanced exon element (20). As illustrated, one iteration of the 5′ enhanced exon element (20) comprises in a 5′ to 3′ order in the following order: a leading untranslated sequence (21), a 5′ affinity tag (22), a 5′ external duplex region (24), a 5′ external spacer (26), and a 3′ intron segment (28).

FIG. 72 depicts various exemplary iterations of the 5′ enhanced exon element (30). As illustrated, one iteration of the 5′ enhanced exon element (30) comprises in a 5′ to 3′ order: a 3′ exon segment (32), a 5′ internal duplex region (34), and a 5′ internal spacer (36).

FIG. 73 depicts various exemplary iterations of the core functional element (40). As illustrated, one iteration of the core functional element (40) comprises a TIE (42), a coding region (46) and a stop region (e.g., a stop codon or stop cassette) (48). Another iteration is illustrated to show the core functional element (47) comprising a noncoding region (47).

FIG. 74 depicts various exemplary iterations of the 3′ enhanced exon element (50). As illustrated, one of the iterations of the 3′ enhanced exon element (50) comprises, in the following 5′ to 3′ order: a 3′ internal spacer (52), a 3′ internal duplex region (54), and a 5′ exon segment (56).

FIG. 75 depicts various exemplary iterations of the 3′ enhanced intron element (60). As illustrated, one of the iterations of the 3′ enhanced intron element (60) comprises, in the following order, a 5′ intron segment (62), a 3′ external spacer (64), a 3′ external duplex region (66), a 3′ affinity tag (68) and a terminal untranslated sequence (69).

FIG. 76 depicts various exemplary iterations a translation initiation element (TIE) (42). TIE (42) sequence as illustrated in one iteration is solely an IRES (43). In another iteration, the TIE (42) is an aptamer (44). In two different iterations, the TIE (42) is an aptamer (44) and IRES (43) combination. In another iteration, the TIE (42) is an aptamer complex (45).

FIG. 77 illustrates an exemplary linear RNA polynucleotide precursor (10) comprising in the following 5′ to 3′ order, a leading untranslated sequence (21), a 5′ affinity tag (22), a 5′ external duplex region (24), a 5′ external spacer (26), a 3′ intron segment (28), a 3′ exon segment (32), a 5′ internal duplex region (34), a 5′ internal spacer (36), a TIE (42), a coding element (46), a stop region (48), a 3′ internal spacer (52), a 3′ internal duplex region (54), a 5′ exon segment (56), a 5′ intron segment (62), a 3′ external spacer (64), a 3′ external duplex region (66), a 3′ affinity tag (68) and a terminal untranslated sequence (69).

FIG. 78 illustrates an exemplary linear RNA polynucleotide precursor (10) comprising in the following 5′ to 3′ order, a leading untranslated sequence (21), a 5′ affinity tag (22), a 5′ external duplex region (24), a 5′ external spacer (26), a 3′ intron segment (28), a 3′ exon segment (32), a 5′ internal duplex region (34), a 5′ internal spacer (36), a coding element (46), a stop region (48), a TIE (42), a 3′ internal spacer (52), a 3′ internal duplex region (54), a 5′ exon segment (56), a 5′ intron segment (62), a 3′ external spacer (64), a 3′ external duplex region (66), a 3′ affinity tag (68) and a terminal untranslated sequence (69).

FIG. 79 illustrates an exemplary linear RNA polynucleotide precursor (10) comprising in the following 5′ to 3′ order, a leading untranslated sequence (21), a 5′ affinity tag (22), a 5′ external duplex region (24), a 5′ external spacer (26), a 3′ intron segment (28), a 3′ exon segment (32), a 5′ internal duplex region (34), a 5′ internal spacer (36), a noncoding element (47), a 3′ internal spacer (52), a 3′ internal duplex region (54), a 5′ exon segment (56), a 5′ intron segment (62), a 3′ external spacer (64), a 3′ external duplex region (66), a 3′ affinity tag (68) and a terminal untranslated sequence (69).

FIG. 80 illustrates the general circular RNA (8) structure formed post splicing. The circular RNA as depicted includes a 5′ exon element (30), a core functional element (40) and a 3′ exon element (50).

FIG. 81 illustrates the various ways an accessory element (70) (e.g., a miRNA binding site) may be included in a linear RNA polynucleotide. FIG. 81A shows a linear RNA polynucleotide comprising an accessory element (70) at the spacer regions. FIG. 81B shows a linear RNA polynucleotide comprising an accessory element (70) located between each of the external duplex regions and the exon segments. FIG. 81C depicts an accessory element (70) within a spacer. FIG. 81D illustrates various iterations of an accessory element (70) located within the core functional element. FIG. 81E illustrates an accessory element (70) located within an internal ribosome entry site (IRES).

FIG. 82 illustrates a screening of a LNP formulated with circular RNA encoding firefly luciferase and having a TIE in primary human (FIG. 82A), mouse (FIG. 82B), and cynomolgus monkey (FIG. 82C) hepatocyte with varying dosages in vitro.

FIGS. 83A-83C illustrates a screening of a LNP formulated with circular RNA encoding firefly luciferase and having a TIE, in primary human hepatocyte from three different donors with varying dosages in vitro.

FIG. 84 illustrates in vitro expression of LNP formulated with circular RNA encoding for GFP and having a TIE, in HeLa, HEK293, and HUH7 human cell models.

FIG. 85 illustrates in vitro expression of LNP formulated with circular RNAs encoding a GFO protein and having a TIE, in primary human hepatocytes.

FIG. 86 illustrates in vitro expression of circular RNA encoding firefly luciferase and having a TIE, in mouse myoblast (FIG. 86A) and primary human muscle myoblast (FIG. 86B) cells.

FIG. 87 illustrates in vitro expression of circular RNA encoding for firefly luciferase and having a TIE, in myoblasts and differentiated primary human skeletal muscle myotubes. FIG. 87A provides the data related to cells received from human donor 1; FIG. 87B provides the data related to cell received from human donor 2.

FIG. 88 illustrates cell-free in vitro translation of circular RNA of variable sizes. In FIG. 88A circular RNA encoding for firefly luciferase and linear mRNA encoding for firefly luciferase was tested for expression. In FIG. 88B, human and mouse cells were given circular RNAs encoding for ATP7B proteins. Some of the circular RNAs tested were codon optimized. Circular RNA expressing firefly luciferase was used for comparison.

FIG. 89 shows an exemplary RNA circularization process. The schematic shown in FIG. 89A depicts an autocatalytic circularization process. Briefly, precursor RNA molecules containing intron segments and accessory elements that enhance circularization efficiency undergo splicing, resulting in a synthetic circular RNA and two excised intron/accessory sequence segments (spliced out intron segments/fragments). Some circularized RNA (oRNA) is nicked during synthesis. FIG. 89B shows an exemplary chromatogram showing peak residence of different species after size exclusion HPLC analysis.

FIG. 90 depicts an exemplary negative selection purification method for circular RNA molecules such as oRNA. Oligonucleotides that are complementary to sequences present in the precursor RNA (such as the intron segments or external accessory regions) but not the oRNA are bound to a solid support, such as a bead. oRNA preparations are washed over the bead; precursor RNA, partially spliced RNA, incomplete transcripts, and post-splicing intron segments bind to the oligonucleotide under certain buffer conditions while oRNA and nicked oRNA flow through. Flowthrough is collected for further processing.

FIG. 91A and FIG. 91B depict an exemplary negative selection purification method for circular RNA molecule such as oRNA. The schematic shown in FIG. 91A depicts enzymatic polyadenylation of in vitro transcription reaction products containing oRNA and linear RNA, resulting in polyadenylation of only the linear RNA. The mixture of linear and circular RNA is washed over beads conjugated to deoxythymidine oligonucleotides (“Oligo dT”) under specific buffer conditions. Polyadenylated linear RNA anneals to the beads while oRNA flows through for collection. FIG. 91B shows exemplary SEC-HPLC chromatograms of in vitro transcription (IVT) reaction products prior to polyadenylation and purification (left panel) and of the eluant following polyadenylation using E. coli polyA polymerase and purification with oligo-dT beads in binding buffer (right panel).

FIG. 92A and FIG. 92B depict an exemplary circular RNA enzymatic purification method. In this method, oRNA is synthesized by IVT in the presence of excess GMP and is autocatalytically spliced during the process. The resulting reaction products are digested with Xrn1 (a 5′ to 3′ exonuclease requiring a 5′ terminal monophosphate) and RNase R (a 3′ to 5′ exonuclease) to remove non-circular RNA molecules. FIG. 92A shows such Xrn1 and RNaseR digestion of linear RNA. FIG. 92B shows exemplary SEC-HPLC chromatograms of IVT reaction products prior to enzymatic digestion (left pane) and of the final, enzymatically purified material (right panel).

FIG. 93A and FIG. 93B show induction of RIG-1 and IFNB1 RNA expression, markers of immune stimulation, following transfection of the cells with the various RNA preparations indicated. All RNA preparations except for the commercially available 3phpRNA were produced using in vitro transcription and circularization of RNA comprising an Anabaena permuted intron, GLuc reading frame, strong homology arms, 5′ and 3′ spacers, and a CVB3 IRES. RIG-1 and IFNB1 RNA expression was measured using RT-qPCR. In FIG. 93, “IVT” indicates an unpurified reaction mixture; “+ GMP” indicates an unpurified reaction mixture in which the in vitro transcription was performed in the presence of 12.5-fold GMP relative to GITP; “+HPLC” indicates a reaction mixture purified by HPLC; “+HPLC/GMP” indicates a reaction mixture purified by HPLC in which the in vitro transcription was performed in the presence of 12.5-fold GMP relative to GTP; “3phpRNA” indicates a positive control comprising a triphosphate hairpin RNA (tlrl-hprna, Invivogen); and “mock” indicates a preparation containing no RNA. FIG. 93A shows immune stimulation of HeLa cells, and FIG. 93B shows immune stimulation of A594 cells.

FIG. 94A and FIG. 94B shows anti-CD19 CAR expression levels resulting from in vitro delivery via electroporation of various circular RNA encoding chimeric antigen receptors in human T cells. FIG. 94A provides representative dot plots from FACs analysis of human T cell expression of CD19-41BBζ, CD19-CD28ζ, HER2-41BBζ, and HER2-CD28ζCARs. FIG. 94B depicts cumulative data for the MFI of CD19-41BBζ, CD19-CD28ζ, HER2-41BBζ, and HER2-CD28ζ expression collected via fluorescence-activated cell sorting (FACS).

FIGS. 95A-95C illustrate cytotoxic response to tumor cells upon electroporation of T cells with circular RNA encoding CD19-41BBζ and CD19-CD28ζ and subsequent co-culture with tumor cells. FIG. 95A provides the % specific lysis of tumor cells after coculture with T cells expressing oRNA encoding CD19-41BBζ, CD19-CD28ζ, HER2-41BBζ, and HER2-CD28ζCARs in comparison to T cells expressing a circular RNA encoding mOX40L.

FIG. 95B and FIG. 95C depict IFN-g and IL-2 cytokine in μg/mL, respectively, secreted by T cells expressing the listed oRNA as compared to a circular RNA encoding mOX40L after co-cultured with tumor cells.

FIG. 96A and FIG. 96B show in vivo mOX40L expression in the splenic and peripheral blood T cells of humanized mice following intravenous administration of LNP formulated with circular RNAs encoding mOX40L. LNPs were formulated with either PBS (indicated as “vehicle” in said figure), or LNP-oRNA constructs formulated with lipid 10b-15, 10a-27, or 10a-26. FIG. 96A depicts mOX40L detection in T cells in the spleen of the humanized mice. FIG. 96B depicts mOX40L detection in T cells in the peripheral blood of the humanized mice.

FIG. 97 illustrates B cell aplasia in humanized mice after intravenous administration of LNP formulated with circular RNA encoding anti-CD19 chimeric antigen receptor (CAR). Representative FACS dot plots from the peripheral blood of untreated animals (left) and treated animals (right) show the percentage of B cells post 6 days from intravenous administration.

FIG. 98A and FIG. 98B show % killing of Nalm6 tumor cells after co-culture with LNP-oRNA encoding CAR or control (FIG. 98A) and chimeric antigen receptor (CAR) surface expression (FIG. 98B) following in vitro transfection of LNP-circular RNA (oRNA) encoding CD19-41BBζ or CD19-CD28ζCARs. FIG. 98A illustrates killing of Nalm6 tumor cells after co-culture of T cells transfected with LNP-oRNA constructs encoding CARs of CD19-41BBζ and CD19-CD28ζCARs along with HER2-41BBz, HER2-CD28z, or the control LN1-oRNA mOX40L. FIG. 98B provides mean fluorescence intensity (MFI) of the CAR surface expression on T cells treated with the LNP-oRNA CAR constructs.

FIG. 99 depicts antigen-dependent tumor regression measured by total flux (in photons/sec) following dosing of mice with either PBS, PBMC, LNP-oRNA encoding for mOx40L, LNP-oRNA encoding for CD19-41BBζ (“CD19-41BBζ is (“CD19-41BBζ is CAR”), oRNA encoding for and CD19-CD28ζ(“CD19-CD28ζ is CAR”), LNP-oRNA encoding for HER2-41BBz CAR (“HER2-41BBz is CAR”), or LNP-oRNA encoding for HER2-CD287 CAR (“HER2-CD28z is CAR”). PBS and PBMC solutions lacking oRNAs were used as negative control.

FIG. 100A, FIG. 100B, and FIG. 100C depict the correlation between IRES activities in myotubes and hepatocytes or myotubes and T cells. Each data point indicates the mean expression value of a circular RNA containing a IRES in front of a Gaussia luciferase coding region, wherein each IRES comprises a sequence selected from SEQ ID NOs: 1-2983 and 3282-3287 or a fragment thereof. Circular RNAs containing the IRESs were synthesized in an array format and formulated into LNPs before being transfected into activated primary human T cells, primary human myotubes, and primary human hepatocytes. All data points are normalized to a positive control IRES (SEQ ID NO: 3282).

FIG. 101A, FIG. 101B, and FIG. 101C depict IRES activities in hepatocytes (FIG. 101A), myotubes (FIG. 101B), and T cells (FIG. 101C) relative to IRESs commonly used (EMCV, CVB3). Each data point indicates the mean expression value of a circular RNA containing a IRES in front of a Gaussia luciferase coding region, wherein each IRES comprises a sequence selected from SEQ ID NOs: 1-2983 and 3282-3287 or a fragment thereof. Circular RNAs containing the IRESs were synthesized in an array format and formulated into LNPs before being transfected into activated primary human T cells, primary human myotubes, and primary human hepatocytes. All data points are normalized to a positive control IRES (SEQ ID NO: 3282).

FIGS. 102A and 102B are scatter plots showing the relative expression of circular RNA (oRNA) encoding a Gaussia luciferase (gLuc) gene expressed in two of three cell types of interest. (FIG. 102A) Expression of gLuc-encoding oRNA encapsulated in a lipid nanoparticle (LNP) and transfected into primary human hepatocytes (PHH) under transcriptional control of an Internal Ribosomal Entry Site (IRES) sequence listed in Tables 5 and 6 versus gLuc expression in T cells electroporated (EP TCL) with a gLuc-encoding oRNA construct under transcriptional control of the same IRES sequence. A strong correlation is apparent between gLuc expression seen in PPH and EP TCL. (FIG. 102B) Expression of gLuc-encoding oRNA encapsulated in a lipid nanoparticle (LNP) and transfected into primary human myotubes (MYO) under transcriptional control of an IRES sequence listed in Tables 5 and 6 versus gLuc expression in EP TCL with a gLuc-encoding oRNA construct under transcriptional control of the same IRES sequence. A strong correlation is apparent between gLuc expression seen in MYO and EP TCL.

FIGS. 103A-103C are scatter plots showing the relative expression of oRNA encoding a Firefly luciferase (FLuc) gene against the expression of a gLuc gene expressed in one of three cell types of interest (PHH, MYO, EP TCL). (FIG. 103A) Expression of FLuc-encoding and gLuc-encoding oRNA encapsulated in a LNP and transfected into primary human myotubes under transcriptional control of an IRES sequence listed in Table 7. (FIG. 103B) Expression of FLuc-encoding and gLuc-encoding oRNA encapsulated in a LNP and transfected into primary human hepatocytes under transcriptional control of an IRES sequence listed in Table 8. (FIG. 103C) Expression of FLuc-encoding and gLuc-encoding oRNA transfected into primary human T cells via electroporation and under transcriptional control of an IRES sequence listed in Table 9. In all three cell types, a strong correlation was observed between gLuc and FLuc expression within the same cell type.

FIG. 104 depicts the estimated number of clones per internal ribosome entry sites (IRES) (“Estimated clones per fragment”) against the number of sequences in each pool (“Pooled Fragments”). There were 11 pools collected. The dotted line in the chart represents the estimated linear relationship between the estimated number of clones per IRES for each sequence.

FIG. 105 provides a chromatographic overlay of the in vitro transcription reaction (IVT) that resulted from the pooled plasmid output of a IRES library screening, and a standard single plasmid control determined using HPLC analysis.

FIG. 106 shows chromatograms of circRNA generated via IVT before and after RNase R digestion. FIG. 106A provides a control circRNA before RNase R digestion. FIG. 106B provides the circular RNA post IVT from the pooled plasmid before RNase R digestion. FIG. 106C provides a control circRNA after RNase R digestion. FIG. 106D provides the circular RNA post IVT from the pooled plasmid output post RNase R digestion.

FIG. 107 illustrates the absorbance profile of the polysome species contained in sucrose gradients collected from cells translating circular RNAs having an internal ribosome entry site (IRES).

FIG. 108 provides a graphical summary of the circRNAs coded for in a plasmid pool, ranked by polysome load, and determined by sequencing read strength of the RNA's contained in the sucrose gradients of interest.

FIG. 109 provides a graphical representation of the RNAs coded for in the pool, ranked by the calculated half-life (in hours) of RNAs after filtering for a minimal count in at least 3 timepoints.

FIG. 110 provides a graphical representation of the RNAs coded for in the pool, ranked by the estimated circularization efficiency, wherein the estimated circularization efficiency was calculated by contrasting the count in the exonuclease treated library to the IVT library after filtering for a minimal count after library size normalization.

FIG. 111 provides the immunoprecipitation results for circular RNA constructs encoding for dystrophin and having either a V5 or vinculin antibody tag. Circular RNA constructs were derived from an IVT reaction of vectors having IRES of either Constructs 82-87 and 81. Each circular RNA construct was tested twice. FIG. 111A shows the immunoprecipitation imaging of the circular RNA. FIG. 111B shows the area under the curve (AUC) values.

FIG. 112 provides the immunoprecipitation results for circular RNA constructs encoding for dystrophin and having either a V5 or vinculin antibody tag. Circular RNA constructs were derived from an IVT reaction of Constructs 81, 85, 88-90. Each circular RNA construct was tested twice. FIG. 112A shows the immunoprecipitation imaging of the circular RNA. FIG. 112B shows the area under the curve (AUC) values generated from the immunoprecipitation results.

FIG. 113 provides the immunoprecipitation results for circular RNA constructs encoding for dystrophin and having either a V5 or vinculin antibody tag. Circular RNA constructs were derived from an IVT reaction of Constructs 81-83, 85-87, 91. Each circular RNA construct was tested twice. FIG. 113A shows the immunoprecipitation imaging of the circular RNA. FIG. 113B shows the area under the curve (AUC) values generated from the immunoprecipitation results.

FIG. 114 provides the immunoprecipitation results for circular RNA constructs encoding for dystrophin and having either a V5 or vinculin antibody tag. Circular RNA constructs were derived from an IVT reaction of Constructs 81-83, 85-89, 91. FIG. 114A shows the immunoprecipitation imaging of the circular RNA. FIG. 114B shows the area under the curve (AUC) values generated from the immunoprecipitation results.

FIG. 115 depicts immunoprecipitation image results of the circular RNA encoding dystrophin that was injected into mdx mice in vivo. Gastrocnemius muscle (“Gastroc”) results are on the right and diaphragm results (“Diaphragm”) are on the left of the figure. Circular RNAs further comprised of an a SEQ ID NO: 3283, SEQ ID NO: 3291, SEQ ID NO: 24894, or SEQ ID NO: 24895 IRES.

FIG. 116 provides the Western Blot results for circular RNA constructs comprising either SEQ ID NO: 3282 (FIG. 116A) and SEQ ID NO: 3291 (FIG. 116B) IRES and encoding a dystrophin protein. FIG. 116C provides the AUC results of the percent dystrophin present.

FIG. 117 provides the fluc expression and expression decay of circular RNAs comprising a IRES (e.g., SEQ ID NO: 1284 or SEQ ID NO: 24896) in the liver (FIG. 117A) or the spleen (FIG. 117B) of mice post administration of the circular RNA.

FIG. 118 provides the hEPO expression and expression decay of circular RNAs comprising an IRES (e.g., SEQ ID NO: 1284, SEQ ID NO: 3291 or SEQ ID NO: 24896) post administration of the circular RNA.

FIG. 119 shows the expression of fluc (FIG. 119A), hEPO (FIG. 119B), or FIX (FIG. 119C) protein expression in primary human hepatocytes post administration of circular RNAs encoding either fluc, hEPO, or FIX and having a SEQ ID NO: 3283, SEQ ID NO: 3282, SEQ ID NO: 1284, SEQ ID NO: 3291 IRES.

### DETAILED DESCRIPTION

The present disclosure provides, among other things, methods and compositions for treating an autoimmune disorder, deficiency disease, or cancer based on circular RNA therapy. In particular, the present disclosure provides methods for treating an autoimmune disorder, deficiency disease, or cancer by administering to a subject in need of treatment a composition comprising a circular RNA encoding at least one therapeutic protein at an effective dose and an administration interval such that at least one symptom or feature of the relevant disease or disorder is reduced in intensity, severity, or frequency or is delayed in onset.

As disclosed herein, the improved circular RNA therapy, along with associated compositions and methods, allows for increased circular RNA stability and expression in target cells (e.g., T cells), among other things. In some embodiments, the circular RNA is transcribed from a linear RNA polynucleotide construct comprising enhanced intron elements, enhanced exon elements, and a core functional element. The enhanced intron element, in some embodiments, comprises post splicing group I intron segments, spacers, duplex sequences, affinity sequences, and unique untranslated sequences that allows for optimal circularization. In some embodiments, the enhanced exon element comprises an exon segment, spacers and duplex sequences to aid with the circularization process and for maintaining stability of the circular RNA post circularization. Within the same embodiments, the core functional element includes the essential elements for protein translation of a translation initiation element (TIE), a coding or noncoding element, and a termination sequence (e.g., a stop codon or stop cassette). Together, the enhanced intron elements, enhanced exon elements, and core functional element comprising a coding element provides an optimal circular RNA polynucleotide for encoding a therapeutic protein. In one embodiment, the enhanced intron elements, enhanced exon elements, and core functional element comprising a noncoding element provides an optimal circular RNA polynucleotide for triggering an immune system as an adjuvant.

Also disclosed herein is a DNA template (e.g., a vector) for making circular RNA. In some embodiments, the DNA template comprises a 3′ enhanced intron segment, a 3′ enhanced exon segment, a core functional element, a 5′ enhanced exon segment, and a 5′ enhanced intron segment. In some embodiments, these elements are positioned in the DNA template in the above order.

Additional embodiments include circular RNA polynucleotides, including circular RNA polynucleotides (e.g., a circular RNA comprising 3′ enhanced exon element, a core functional element, and a 5′ enhanced exon element) made using the DNA template provided herein, compositions comprising such circular RNA, cells comprising such circular RNA, methods of using and making such DNA template, circular RNA, compositions and cells.

In some embodiments, provided herein are methods comprising administration of circular RNA polynucleotides provided herein into cells for therapy or production of useful proteins. In some embodiments, the method is advantageous in providing the production of a desired polypeptide inside eukaryotic cells with a longer half-life than linear RNA, due to the resistance of the circular RNA to ribonucleases.

Circular RNA polynucleotides lack the free ends necessary for exonuclease-mediated degradation, causing them to be resistant to several mechanisms of RNA degradation and granting extended half-lives when compared to an equivalent linear RNA. Circularization may allow for the stabilization of RNA polynucleotides that generally suffer from short half-lives and may improve the overall efficacy of exogenous mRNA in a variety of applications. In an embodiment, the functional half-life of the circular RNA polynucleotides provided herein in eukaryotic cells (e.g., mammalian cells, such as human cells) as assessed by protein synthesis is at least 20 hours (e.g., at least 80 hours).

Various aspects of the invention are described in detail in the following sections. The use of sections is not meant to limit the invention. Each section can apply to any aspect of the invention. In this application, the use of “or” means “and/or” unless stated otherwise.

### 1. Definitions

Linear nucleic acid molecules are said to have a “5′-terminus” (or “5′ end”) and a “3′-terminus” (or “3′ end”) because nucleic acid phosphodiester linkages occur at the 5′ carbon and 3′ carbon of the sugar moieties of the substituent mononucleotides. The end nucleotide of a polynucleotide at which a new linkage would be to a 5′ carbon is its 5′ terminal nucleotide. The end nucleotide of a polynucleotide at which a new linkage would be to a 3′ carbon is its 3′ terminal nucleotide. A “terminal nucleotide,” as used herein, is the nucleotide at the end position of the 3′- or 5′-terminus.

As used herein, the term “3′ group I intron segment” or “3′ group I intron fragment” refers to a sequence with 75% or higher similarity to the 3′-proximal end of a natural group I intron including the splice site dinucleotide and optionally a stretch of natural exon sequence. In some embodiments, a circular RNA comprises a post splicing 3′ group I intron fragment. In some embodiments, the post splicing 3′ group I intron fragment in the circular RNA is a post splicing stretch of exon sequence. In some embodiments, the circular RNA further comprises a desired expression sequence, and the post splicing stretch of exon sequence is (e.g., designed) to be a portion of the desired expression sequence, contiguous with the desired expression sequence, and/or in frame with the desired expression sequence.

As used herein, the term “5′ group I intron segment” or “5′ group I intron fragment” refers to a sequence with 75% or higher similarity to the 5′-proximal end of a natural group I intron including the splice site dinucleotide and optionally a stretch of natural exon sequence. In some embodiments, a circular RNA comprises a post splicing 5′ group I intron fragment. In some embodiments, the post splicing 5′ group I intron fragment in the circular RNA is a post splicing stretch of exon sequence. In some embodiments, the circular RNA further comprises a desired expression sequence, and the post splicing stretch of exon sequence is (e.g., designed) to be a portion of the desired expression sequence, contiguous with the desired expression sequence, and/or in frame with the desired expression sequence.

As used herein, the term “permutation site” refers to the site in a group I intron where a cut is made prior to permutation of the intron. This cut generates 3′ and 5′ group I intron segments that are permuted to be on either side of a stretch of precursor RNA to be circularized.

As used herein, the singular forms “a,” “an,” and “the” include plural referents unless the content clearly dictates otherwise. Thus, for example, reference to “a cell” includes combinations of two or more cells, or entire cultures of cells; reference to “a polynucleotide” includes, as a practical matter, many copies of that polynucleotide.

Unless specifically stated or obvious from context, as used herein, the term “about,” is understood as within a range of normal tolerance in the art, for example within 2 standard deviations of the mean. “About” can be understood as within 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% of the stated value. Unless otherwise clear from the context, all numerical values provided herein are modified by the term “about.”

As used herein, an “affinity sequence” or “affinity tag” is a region of a polynucleotide sequence ranging from one (1) nucleotide to hundreds or thousands of nucleotides containing a repeated set of nucleotides for the purposes of aiding purification of a polynucleotide sequence. For example, an affinity sequence may comprise, but is not limited to, a polyA or polyAC sequence. In some embodiments, affinity tags are used in purification methods, referred to herein as “affinity-purification,” in which selective binding of a binding agent to molecules comprising an affinity tag facilitates separation from molecules that do not comprise an affinity tag. In some embodiments, an affinity-purification method is a “negative selection” purification method, in which unwanted species, such as linear RNA, are selectively bound and removed and wanted species, such as circular RNA, are eluted and separated from unwanted species.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain may comprise a heavy chain variable region (abbreviated herein as VH) and a heavy chain constant region. The heavy chain constant region can comprise three constant domains, CH1, CH2 and CH3. Each light chain can comprise a light chain variable region (abbreviated herein as VL) and a light chain constant region. The light chain constant region can comprise one constant domain, CL. The VH and VL regions may be further subdivided into regions of hypervariability, termed complementarity determining regions (CDRs), interspersed with regions that are more conserved, termed framework regions (FR). Each VH and VL may comprise three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component of the classical complement system. Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain variable fragments (scFv), camelized antibodies, affybodies, Fab fragments, F(ab′)2 fragments, disulfide-linked variable fragments (sdFv), anti-idiotypic (anti-id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures. The term “Kabat numbering” and like terms are recognized in the art and refer to a system of numbering amino acid residues in the heavy and light chain variable regions of an antibody, or an antigen-binding molecule thereof. In certain aspects, the CDRs of an antibody may be determined according to the Kabat numbering system (see, e.g., Kabat EA & Wu TT (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally may include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme. In certain aspects, the CDRs of an antibody may be determined according to the Chothia numbering scheme, which refers to the location of immunoglobulin structural loops (see, e.g., Chothia C & Lesk AM, (1987), J Mol Biol 196: 901-917; A1-Lazikani B et al, (1997) J Mol Biol 273: 927-948; Chothia C et al., (1992) J Mol Biol 227: 799-817; Tramontano A et al, (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35a is present, the loop ends at 33; if both 35A and 35B are present, the loop ends at 34). In a specific embodiment, the CDRs of the antibodies described herein have been determined according to the Chothia numbering scheme.

As used herein, the term “variable region” or “variable domain” is used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some embodiments, the variable region is a human variable region. In some embodiments, the variable region comprises rodent or murine CDRs and human framework regions (FRs). In particular embodiments, the variable region is a primate (e.g., non-human primate) variable region. In some embodiments, the variable region comprises rodent or murine CDRs and primate (e.g., non-human primate) framework regions (FRs). The terms “VL” and “VL domain” are used interchangeably to refer to the light chain variable region of an antibody or an antigen-binding molecule thereof. The terms “VH” and “VH domain” are used interchangeably to refer to the heavy chain variable region of an antibody or an antigen-binding molecule thereof.

As used herein, the terms “constant region” and “constant domain” are interchangeable and have a meaning common in the art. The constant region is an antibody portion, e.g., a carboxyl terminal portion of a light and/or heavy chain which is not directly involved in binding of an antibody to an antigen but which may exhibit various effector functions, such as interaction with the Fc receptor. The constant region of an immunoglobulin molecule generally has a more conserved amino acid sequence relative to an immunoglobulin variable domain.

As used herein, “aptamer” refers in general to either an oligonucleotide of a single defined sequence or a mixture of said nucleotides, wherein the mixture retains the properties of binding specifically to the target molecule (e.g., eukaryotic initiation factor, 40S ribosome, polyC binding protein, polyA binding protein, polypyrimidine tract-binding protein, argonaute protein family, Heterogeneous nuclear ribonucleoprotein K and La and related RNA-binding protein). Thus, as used herein “aptamer” denotes both singular and plural sequences of nucleotides, as defined hereinabove. The term “aptamer” is meant to refer to a single- or double-stranded nucleic acid which is capable of binding to a protein or other molecule. In general, aptamers preferably comprise about 10 to about 100 nucleotides, preferably about 15 to about 40 nucleotides, more preferably about 20 to about 40 nucleotides, in that oligonucleotides of a length that falls within these ranges are readily prepared by conventional techniques. Optionally, aptamers can further comprise a minimum of approximately 6 nucleotides, preferably 10, and more preferably 14 or 15 nucleotides, that are necessary to effect specific binding. As used herein, the phrase “aptamer complex” refers to a sequence comprising two or more aptamers operably connected to one another. An aptamer complex can include two or more aptamer complexes the sequences of which are contiguous with one another or may have one or more intervening nucleotides between each of the two or more aptamers.

As used herein, “autoimmunity” is defined as persistent and progressive immune reactions to non-infectious self-antigens, as distinct from infectious non self-antigens from bacterial, viral, fungal, or parasitic organisms which invade and persist within mammals and humans. Autoimmune conditions include scleroderma, Grave's disease, Crohn's disease, Sjorgen's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, and thyroiditis, as well as in the generalized autoimmune diseases typified by human Lupus. “Autoantigen” or “self-antigen” as used herein refers to an antigen or epitope which is native to the mammal and which is immunogenic in said mammal.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

As used herein, the terms “circRNA,” “circular polyribonucleotide,” “circular RNA,” “circularized RNA,” and “oRNA” are used interchangeably and refer to a single-stranded RNA polynucleotide wherein the 3′ and 5′ ends that are normally present in a linear RNA polynucleotide have been (e.g., covalently) joined together. As used herein, such terms also include preparations comprising circRNAs. Also encompassed within the meaning of these terms are precursor RNA polynucleotides capable of circularizing to produce a covalently-closed circular RNA molecule. Such precursor RNA polynucleotides may include sequence elements that mediate or facilitate circularization of the polynucleotide, including group I intron segments (e.g., 3′ and 5′ group I intron segments), spacer sequences, internal duplex regions, polyA sequences, polyC sequences, polyAC sequences, polypyrimidine tract, among others.

As used herein, the term “circularization efficiency” refers to a measurement of the rate of formation of amount of resultant circular polyribonucleotide as compared to its linear starting material.

The expression sequences in the polynucleotide construct may be separated by a “cleavage site” sequence which enables polypeptides encoded by the expression sequences, once translated, to be expressed separately by the cell. A “self-cleaving peptide” refers to a peptide which is translated without a peptide bond between two adjacent amino acids, or functions such that when the polypeptide comprising the proteins and the self-cleaving peptide is produced, it is immediately cleaved or separated into distinct and discrete first and second polypeptides without the need for any external cleavage activity.

As used herein, “coding element,” “coding sequence,” “coding nucleic acid,” or “coding region” is region located within the expression sequence and encodings for one or more proteins or polypeptides (e.g., therapeutic protein). As used herein, a “noncoding element,” “noncoding sequence,” “non-coding nucleic acid,” or “noncoding nucleic acid” is a region located within the expression sequence. This sequence, but itself does not encode for a protein or polypeptide, but may have other regulatory functions, including but not limited, allow the overall polynucleotide to act as a biomarker or adjuvant to a specific cell.

As used herein, the term “DNA template” refers to a DNA sequence capable of transcribing a linear RNA polynucleotide. For example, but not intending to be limiting, a DNA template may include a DNA vector, PCR product or plasmid.

As used herein, the terms “duplexed,” “double-stranded,” and “hybridized” are used interchangeably and refer to double-stranded nucleic acids formed by hybridization of two single strands of nucleic acids containing complementary sequences. Sequences of the two single-stranded nucleic acids can be fully complementary or partially complementary. In some embodiments, a nucleic acid provided herein may be fully double-stranded or partially double-stranded. In most cases, genomic DNA is double-stranded.

As used herein, two “duplex sequences,” “duplex forming sequences,” “duplex region,” “duplex forming regions,” “homology arms,” or “homology regions,” complement, or are complementary, fully or partially, to one another when the two regions share a sufficient level of sequence identity to one another's reverse complement to act as substrates for a hybridization reaction. In some embodiments, two duplex forming sequences are thermodynamically favored to cross-pair in a sequence specific interaction. As used herein, polynucleotide sequences have “homology” when they are either identical or share sequence identity to a reverse complement or “complementary” sequence. The percent sequence identity between a homology region and a counterpart homology region's reverse complement can be any percent of sequence identity that allows for hybridization to occur. In some embodiments, an internal duplex forming region of a polynucleotide disclosed herein is capable of forming a duplex with another internal duplex forming region and does not form a duplex with an external duplex forming region.

As used herein, the term “encode” refers broadly to any process whereby the information in a polymeric macromolecule is used to direct the production of a second molecule that is different from the first. The second molecule may have a chemical structure that is different from the chemical nature of the first molecule. For example, a DNA template (e.g., a DNA vector) may encode a RNA polynucleotide; a precursor RNA polynucleotide (e.g., a linear precursor RNA polynucleotide) may encode a mature RNA polynucleotide (e.g., a circular RNA polynucleotide).

As used herein, the term “expression sequence” refers to a nucleic acid sequence that encodes a product, e.g., a peptide or polypeptide, regulatory nucleic acid, or non-coding nucleic acid. An exemplary expression sequence that codes for a peptide or polypeptide can comprise a plurality of nucleotide triads, each of which can code for an amino acid and is termed as a “codon.”

As used herein, an “internal ribosome entry site” or “IRES” refers to an RNA sequence or structural element ranging in size from 10 nt to 1000 nt or more, capable of initiating translation of a polypeptide in the absence of a typical RNA cap structure. An IRES is typically about 500 nt to about 700 nt in length. The IRES may comprise naturally occurring sequences and/or synthetic, non-naturally occurring sequences.

As used herein, a “leading untranslated sequence” is a region of polynucleotide sequences ranging from 1 nucleotide to hundreds of nucleotides located at the upmost 5′ end of a polynucleotide sequence. The sequences can be defined or can be random. An leading untranslated sequence is non-coding. As used herein, a “terminal untranslated sequence” is a region of polynucleotide sequences ranging from 1 nucleotide to hundreds of nucleotides located at the downmost 3′ end of a polynucleotide sequence. The sequences can be defined or can be random. A terminal untranslated sequence is non-coding.

As used herein, a “miRNA site” refers to a stretch of nucleotides within a polynucleotide that is capable of forming a duplex with at least 8 nucleotides of a natural miRNA sequence.

The term “nucleotide” refers to a ribonucleotide, a deoxyribonucleotide, a modified form thereof, or an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5′-position pyrimidine modifications, 8′-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine; sugars such as 2′-methyl ribose; non-natural phosphodiester linkages such as methylphosphonate, phosphorothioate and peptide linkages. Nucleotide analogs include 5-methoxyuridine, 1-methylpseudouridine, and 6-methyladenosine.

All nucleotide sequences disclosed herein can represent an RNA sequence or a corresponding DNA sequence. It is understood that deoxythymidine (dT or T) in a DNA is transcribed into a uridine (U) in an RNA. As such, “T” and “U” are used interchangeably herein in nucleotide sequences.

The terms “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, or up to about 10,000 or more bases, composed of nucleotides, e.g., deoxyribonucleotides or ribonucleotides, and may be produced enzymatically or synthetically (e.g., as described in U.S. Pat. No. 5,948,902 and the references cited therein), which can hybridize with naturally occurring nucleic acids in a sequence specific manner analogous to that of two naturally occurring nucleic acids, e.g., can participate in Watson-Crick base pairing interactions. An “oligonucleotide” is a polynucleotide comprising fewer than 1000 nucleotides, such as a polynucleotide comprising fewer than 500 nucleotides or fewer than 100 nucleotides. Naturally occurring nucleic acids are comprised of nucleotides, including guanine, cytosine, adenine, thymine, and uracil containing nucleotides (G, C, A, T, and U respectively). As used herein, “polyA” means a polynucleotide or a portion of a polynucleotide consisting of nucleotides comprising adenine. As used herein, “polyT” means a polynucleotide or a portion of a polynucleotide consisting of nucleotides comprising thymine. As used herein, “polyAC” means a polynucleotide or a portion of a polynucleotide consisting of nucleotides comprising adenine or cytosine.

As used herein, the term “ribosomal skipping element” refers to a nucleotide sequence encoding a short peptide sequence capable of causing generation of two peptide chains from translation of one RNA molecule. While not wishing to be bound by theory, it is hypothesized that ribosomal skipping elements function by (1) terminating translation of the first peptide chain and re-initiating translation of the second peptide chain; or (2) cleavage of a peptide bond in the peptide sequence encoded by the ribosomal skipping element by an intrinsic protease activity of the encoded peptide, or by another protease in the environment (e.g., cytosol).

The term “sequence identity,” as used herein, refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

As used herein, a “spacer” refers to a region of a polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex regions.

As used herein, the term “stop cassette” refers to one or more stop codons present in two or more open reading frames.

As used herein, the term “splice site” refers to a dinucleotide that is partially or fully included in a group I intron and between which a phosphodiester bond is cleaved during RNA circularization.

As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

As used herein, the terms “transfect” or “transfection” refer to the intracellular introduction of one or more encapsulated materials (e.g., nucleic acids and/or polynucleotides) into a cell, or preferably into a target cell. The term “transfection efficiency” refers to the relative amount of such encapsulated material (e.g., polynucleotides) up-taken by, introduced into and/or expressed by the target cell which is subject to transfection. In some embodiments, transfection efficiency may be estimated by the amount of a reporter polynucleotide product produced by the target cells following transfection. In some embodiments, a transfer vehicle has high transfection efficiency. In some embodiments, a transfer vehicle has at least about 10%, 20%, 30%, 40%, 50%, 60%, 70%, 80%, or 90% transfection efficiency.

As used herein, “transfer vehicle” includes any of the standard pharmaceutical carriers, diluents, excipients, and the like, which are generally intended for use in connection with the administration of biologically active agents, including nucleic acids. In certain embodiments of the present invention, the transfer vehicles (e.g., lipid nanoparticles) are prepared to encapsulate one or more materials or therapeutic agents (e.g., circRNA). The process of incorporating a desired therapeutic agent (e.g., circRNA) into a transfer vehicle is referred to herein as or “loading” or “encapsulating” (Lasic, et al., FEBS Lett., 312: 255-258, 1992). The transfer vehicle-loaded or -encapsulated materials (e.g., circRNA) may be completely or partially located in the interior space of the transfer vehicle, within a bilayer membrane of the transfer vehicle, or associated with the exterior surface of the transfer vehicle.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. The treatment or prevention provided by the method disclosed herein can include treatment or prevention of one or more conditions or symptoms of the disease. Also, for purposes herein, “prevention” can encompass delaying the onset of the disease, or a symptom or condition thereof. As used herein, the terms “upstream” and “downstream” refer to relative positions of genetic code, e.g., nucleotides, sequence elements, in polynucleotide sequences. In some embodiments, in an RNA polynucleotide, upstream is toward the 5′ end of the polynucleotide and downstream is toward the 3′ end. In some embodiments, in a DNA polynucleotide, upstream is toward the 5′ end of the coding strand for the gene in question and downstream is toward the 3′ end. A person of ordinary skill in the art would readily understand the meaning of “upstream” and “downstream” sequences as used in the present disclosure.

### A. Lipid Definitions

As used herein, the phrase “biodegradable lipid” or “degradable lipid” refers to any of a number of lipid species that are broken down in a host environment on the order of minutes, hours, or days ideally making them less toxic and unlikely to accumulate in a host over time. Common modifications to lipids include ester bonds, and disulfide bonds among others to increase the biodegradability of a lipid.

As used herein, the phrase “biodegradable PEG lipid” or “degradable PEG lipid” refers to any of a number of lipid species where the PEG molecules are cleaved from the lipid in a host environment on the order of minutes, hours, or days ideally making them less immunogenic. Common modifications to PEG lipids include ester bonds, and disulfide bonds among others to increase the biodegradability of a lipid.

As used herein, the term “cationic lipid” or “ionizable lipid” refers to any of a number of lipid species that carry a net positive charge at a selected pH, such as physiological pH 4 and a neutral charge at other pHs such as physiological pH 7.

As used herein, the term “PEG” means any polyethylene glycol or other polyalkylene ether polymer.

As generally defined herein, a “PEG-OH lipid” (also referred to herein as “hydroxy-PEGylated lipid”) is a PEGylated lipid having one or more hydroxyl (—OH) groups on the lipid.

As used herein, a “phospholipid” is a lipid that includes a phosphate moiety and one or more carbon chains, such as unsaturated fatty acid chains.

As used herein, the term “structural lipid” refers to sterols and also to lipids containing sterol moieties. As defined herein, “sterols” are a subgroup of steroids consisting of steroid alcohols.

The terms “head-group” and “tail-group,” when used herein to describe the compounds (e.g., lipids) of the present invention, and in particular functional groups that are comprised in such compounds, are used for ease of reference to describe the orientation of such compounds or of one or more functional groups relative to other functional groups. For example, in certain embodiments, a hydrophilic head-group (e.g., guanidinium) is bound (e.g., by one or more of hydrogen-bonds, van der Waals' forces, ionic interactions and covalent bonds) to a cleavable functional group (e.g., a disulfide group), which in turn is bound to a hydrophobic tail-group (e.g., cholesterol). In certain embodiments, the compounds disclosed herein comprise, for example, at least one hydrophilic head-group and at least one hydrophobic tail-group, each bound to at least one cleavable group, thereby rendering such compounds amphiphilic.

As used herein, the term “amphiphilic” means the ability to dissolve in both polar (e.g., water) and non-polar (e.g., lipid) environments. For example, in certain embodiments, the compounds (e.g., lipids) disclosed herein comprise at least one lipophilic tail-group (e.g., cholesterol or a C₆₋₂₀ alkyl) and at least one hydrophilic head-group (e.g., imidazole), each bound to a cleavable group (e.g., disulfide).

As used herein, the term “hydrophilic” is used to indicate in qualitative terms that a functional group is water-preferring, and typically such groups are water-soluble. For example, disclosed herein are compounds (e.g., ionizable lipids) that comprise a cleavable group (e.g., a disulfide (S—S) group) bound to one or more hydrophilic groups (e.g., a hydrophilic head-group), wherein such hydrophilic groups comprise or are selected from the group consisting of imidazole, guanidinium, amino, imine, enamine, an optionally-substituted alkyl amino (e.g., an alkyl amino such as dimethylamino) and pyridyl.

As used herein, the term “hydrophobic” is used to indicate in qualitative terms that a functional group is water-avoiding, and typically such groups are not water soluble. In certain embodiments, at least one of the functional groups of moieties that comprise the compounds disclosed herein is hydrophobic in nature (e.g., a hydrophobic tail-group comprising a naturally occurring lipid such as cholesterol). For example, disclosed herein are compounds (e.g., ionizable lipids) that comprise a cleavable functional group (e.g., a disulfide (S—S) group) bound to one or more hydrophobic groups, wherein such hydrophobic groups may comprise, or may be selected from, one or more naturally occurring lipids such as cholesterol, an optionally substituted, variably saturated or unsaturated C₆-C₂₀ alkyl, and/or an optionally substituted, variably saturated or unsaturated C₆-C₂₀ acyl.

As used herein, the term “liposome” generally refers to a vesicle composed of lipids (e.g., amphiphilic lipids) arranged in one or more spherical bilayer or bilayers. Such liposomes may be unilamellar or multilamellar vesicles which have a membrane formed from a lipophilic material and an aqueous interior that contains the encapsulated circRNA to be delivered to one or more target cells, tissues and organs.

As used herein, the phrase “lipid nanoparticle” refers to a transfer vehicle comprising one or more cationic or ionizable lipids, stabilizing lipids, structural lipids, and helper lipids.

In certain embodiments, the compositions described herein comprise one or more liposomes or lipid nanoparticles. Examples of suitable lipids (e.g., ionizable lipids) that may be used to form the liposomes and lipid nanoparticles contemplated include one or more of the compounds disclosed herein.

In some embodiments, a lipid, e.g., an ionizable lipid, disclosed herein comprises one or more cleavable groups. The terms “cleave” and “cleavable” are used herein to mean that one or more chemical bonds (e.g., one or more of covalent bonds, hydrogen-bonds, van der Waals' forces and/or ionic interactions) between atoms in or adjacent to the subject functional group are broken (e.g., hydrolyzed) or are capable of being broken upon exposure to selected conditions (e.g., upon exposure to enzymatic conditions). In certain embodiments, the cleavable group is a disulfide functional group, and in particular embodiments is a disulfide group that is capable of being cleaved upon exposure to selected biological conditions (e.g., intracellular conditions). In certain embodiments, the cleavable group is an ester functional group that is capable of being cleaved upon exposure to selected biological conditions. For example, the disulfide groups may be cleaved enzymatically or by a hydrolysis, oxidation or reduction reaction. Upon cleavage of such disulfide functional group, the one or more functional moieties or groups (e.g., one or more of a head-group and/or a tail-group) that are bound thereto may be liberated. Exemplary cleavable groups may include, but are not limited to, disulfide groups, ester groups, ether groups, and any derivatives thereof (e.g., alkyl and aryl esters). In certain embodiments, the cleavable group is not an ester group or an ether group. In some embodiments, a cleavable group is bound (e.g., bound by one or more of hydrogen-bonds, van der Waals' forces, ionic interactions and covalent bonds) to one or more functional moieties or groups (e.g., at least one head-group and at least one tail-group). In certain embodiments, at least one of the functional moieties or groups is hydrophilic (e.g., a hydrophilic head-group comprising one or more of imidazole, guanidinium, amino, imine, enamine, optionally-substituted alkyl amino and pyridyl).

### B. Chemical Definitions

When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, the term “alkyl” refers to both straight and branched chain C₁₋₄₀ hydrocarbons (e.g., C₆₋₂₀ hydrocarbons), and include both saturated and unsaturated hydrocarbons. In certain embodiments, the alkyl may comprise one or more cyclic alkyls and/or one or more heteroatoms such as oxygen, nitrogen, or sulfur and may optionally be substituted with substituents (e.g., one or more of alkyl, halo, alkoxyl, hydroxy, amino, aryl, ether, ester or amide). In certain embodiments, a contemplated alkyl includes (9Z,12Z)-octadeca-9,12-dien. The use of designations such as, for example, “C₆₋₂₀” is intended to refer to an alkyl (e.g., straight or branched chain and inclusive of alkenes and alkyls) having the recited range carbon atoms. In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂-6 alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene” group, it is understood that the range or number refers to the range or number of carbons in the linear carbon divalent chain. “Alkylene,” “alkenylene,” and “alkynylene” groups may be substituted or unsubstituted with one or more substituents as described herein.

The term “alkoxy,” as used herein, refers to an alkyl group which is attached to another moiety via an oxygen atom (—O(alkyl)). Non-limiting examples include e.g., methoxy, ethoxy, propoxy, and butoxy.

As used herein, the term “aryl” refers to aromatic groups (e.g., monocyclic, bicyclic and tricyclic structures) containing six to ten carbons in the ring portion. The aryl groups may be optionally substituted through available carbon atoms and in certain embodiments may include one or more heteroatoms such as oxygen, nitrogen or sulfur. In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl).

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈ cycloalkyl,” derived from a cycloalkane. Exemplary cycloalkyl groups include, but are not limited to, cyclohexanes, cyclopentanes, cyclobutanes and cyclopropanes.

As used herein, “cyano” refers to —CN.

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” may be used interchangeably.

The terms “halo” and “halogen” as used herein refer to an atom selected from fluorine (fluoro, F), chlorine (chloro, Cl), bromine (bromo, Br), and iodine (iodo, I). In certain embodiments, the halo group is either fluoro or chloro.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C1-4alkyl)4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The invention additionally encompasses compounds described herein as individual isomers substantially free of other isomers, and alternatively, as mixtures of various isomers.

In certain embodiments, the compounds (e.g., ionizable lipids) and the transfer vehicles (e.g., lipid nanoparticles) of which such compounds are a component exhibit an enhanced (e.g., increased) ability to transfect one or more target cells. Accordingly, also provided herein are methods of transfecting one or more target cells. Such methods generally comprise the step of contacting the one or more target cells with the compounds and/or pharmaceutical compositions disclosed herein such that the one or more target cells are transfected with the circular RNA encapsulated therein.

It is to be understood that the terminology used herein is for the purpose of describing particular embodiments only, and is not intended to be limiting. Unless specifically stated or obvious from context, as used herein, the term “or” is understood to be inclusive. Unless defined herein and below in the reminder of the specification, all technical and scientific terms used herein have the same meaning as commonly understood by one of ordinary skill in the art to which the invention pertains. 2. DNA TEMPLATE, PRECURSOR RNA & CIRCULAR RNA

According to the present invention, transcription of a DNA template provided herein (e.g., comprising a 3′ enhanced intron element, 3′ enhanced exon element, a core functional element, a 5′ enhanced exon element, and a 5′ enhanced intron element) results in formation of a precursor linear RNA polynucleotide capable of circularizing. In some embodiments, this DNA template comprises a vector, PCR product, plasmid, minicircle DNA, cosmid, artificial chromosome, complementary DNA (cDNA), extrachromosomal DNA (ecDNA), or a fragment therein. In certain embodiments, the minicircle DNA may be linearized or non-linearized. In certain embodiments, the plasmid may be linearized or non-linearized. In some embodiments, the DNA template may be single-stranded. In other embodiments, the DNA template may be double-stranded. In some embodiments, the DNA template comprises in whole or in part from a viral, bacterial or eukaryotic vector.

The present invention, as provided herein, comprises a DNA template that shares the same sequence as the precursor linear RNA polynucleotide prior to splicing of the precursor linear RNA polynucleotide (e.g., a 3′ enhanced intron element, a 3′ enhanced exon element, a core functional element, and a 5′ enhanced exon element, a 5′ enhanced intron element). In some embodiments, said linear precursor RNA polynucleotide undergoes splicing leading to the removal of the 3′ enhanced intron element and 5′ enhanced intron element during the process of circularization. In some embodiments, the resulting circular RNA polynucleotide lacks a 3′ enhanced intron segment and a 5′ enhanced intron segment, but maintains a 3′ enhanced exon segment, a core functional element, and a 5′ enhanced exon element.

In some embodiments, the precursor linear RNA polynucleotide circularizes when incubated in the presence of one or more guanosine nucleotides or nucleoside (e.g., GTP) and a divalent cation (e.g., Mg²⁺). In some embodiments, the 3′ enhanced exon element, 5′ enhanced exon element, and/or core functional element in whole or in part promotes the circularization of the precursor linear RNA polynucleotide to form the circular RNA polynucleotide provided herein.

In certain embodiments, circular RNA provided herein is produced inside a cell. In some embodiments, precursor RNA is transcribed using a DNA template (e.g., in some embodiments, using a vector provided herein) in the cytoplasm by a bacteriophage RNA polymerase, or in the nucleus by host RNA polymerase II and then circularized.

In certain embodiments, the circular RNA provided herein is injected into an animal (e.g., a human), such that a polypeptide encoded by the circular RNA molecule is expressed inside the animal.

In some embodiments, the DNA (e.g., vector), linear RNA (e.g., precursor RNA), and/or circular RNA polynucleotide provided herein is between 300 and 10000, 400 and 9000, 500 and 8000, 600 and 7000, 700 and 6000, 800 and 5000, 900 and 5000, 1000 and 5000, 1100 and 5000, 1200 and 5000, 1300 and 5000, 1400 and 5000, and/or 1500 and 5000 nucleotides in length. In some embodiments, the polynucleotide is at least 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, or 5000 nt in length. In some embodiments, the polynucleotide is no more than 3000 nt, 3500 nt, 4000 nt, 4500 nt, 5000 nt, 6000 nt, 7000 nt, 8000 nt, 9000 nt, or 10000 nt in length. In some embodiments, the length of a DNA, linear RNA, and/or circular RNA polynucleotide provided herein is about 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, 5000 nt, 6000 nt, 7000 nt, 8000 nt, 9000 nt, or 10000 nt.

In some embodiments, the circular RNA provided herein has higher functional stability than mRNA comprising the same expression sequence. In some embodiments, the circular RNA provided herein has higher functional stability than mRNA comprising the same expression sequence, 5moU modifications, an optimized UTR, a cap, and/or a polyA tail.

In some embodiments, the circular RNA polynucleotide provided herein has a functional half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a functional half-life of 5-80, 10-70, 15-60, and/or 20-50 hours. In some embodiments, the circular RNA polynucleotide provided herein has a functional half-life greater than (e.g., at least 1.5-fold greater than, at least 2-fold greater than) that of an equivalent linear RNA polynucleotide encoding the same protein. In some embodiments, functional half-life can be assessed through the detection of functional protein synthesis.

In some embodiments, the circular RNA polynucleotide provided herein has a half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a half-life of 5-80, 10-70, 15-60, and/or 20-50 hours. In some embodiments, the circular RNA polynucleotide provided herein has a half-life greater than (e.g., at least 1.5-fold greater than, at least 2-fold greater than) that of an equivalent linear RNA polynucleotide encoding the same protein. In some embodiments, the circular RNA polynucleotide, or pharmaceutical composition thereof, has a functional half-life in a human cell greater than or equal to that of a pre-determined threshold value. In some embodiments the functional half-life is determined by a functional protein assay. For example in some embodiments, the functional half-life is determined by an in vitro luciferase assay, wherein the activity of Gaussia luciferase (GLuc) is measured in the media of human cells (e.g. HepG2) expressing the circular RNA polynucleotide every 1, 2, 6, 12, or 24 hours over 1, 2, 3, 4, 5, 6, 7, or 14 days. In other embodiments, the functional half-life is determined by an in vivo assay, wherein levels of a protein encoded by the expression sequence of the circular RNA polynucleotide are measured in patient serum or tissue samples every 1, 2, 6, 12, or 24 hours over 1, 2, 3, 4, 5, 6, 7, or 14 days. In some embodiments, the pre-determined threshold value is the functional half-life of a reference linear RNA polynucleotide comprising the same expression sequence as the circular RNA polynucleotide.

In some embodiments, the circular RNA provided herein may have a higher magnitude of expression than equivalent linear mRNA, e.g., a higher magnitude of expression 24 hours after administration of RNA to cells. In some embodiments, the circular RNA provided herein has a higher magnitude of expression than mRNA comprising the same expression sequence, 5moU modifications, an optimized UTR, a cap, and/or a polyA tail.

In some embodiments, the circular RNA provided herein may be less immunogenic than an equivalent mRNA when exposed to an immune system of an organism or a certain type of immune cell. In some embodiments, the circular RNA provided herein is associated with modulated production of cytokines when exposed to an immune system of an organism or a certain type of immune cell. For example, in some embodiments, the circular RNA provided herein is associated with reduced production of IFN-β1, RIG-I, IL-2, IL-6, IFNγ, and/or TNFα when exposed to an immune system of an organism or a certain type of immune cell as compared to mRNA comprising the same expression sequence. In some embodiments, the circular RNA provided herein is associated with less IFN-β1, RIG-I, IL-2, IL-6, IFNγ, and/or TNFα transcript induction when exposed to an immune system of an organism or a certain type of immune cell as compared to mRNA comprising the same expression sequence. In some embodiments, the circular RNA provided herein is less immunogenic than mRNA comprising the same expression sequence. In some embodiments, the circular RNA provided herein is less immunogenic than mRNA comprising the same expression sequence, 5moU modifications, an optimized UTR, a cap, and/or a polyA tail.

In certain embodiments, the circular RNA provided herein can be transfected into a cell as is, or can be transfected in DNA vector form and transcribed in the cell. Transcription of circular RNA from a transfected DNA vector can be via added polymerases or polymerases encoded by nucleic acids transfected into the cell, or preferably via endogenous polymerases.

### A. Enhanced Intron Elements & Enhanced Exon Elements

Polynucleotides provided herein may comprise one or more enhanced intron elements and/or one or more enhanced exon elements. In some embodiments, the enhanced intron elements and enhanced exon elements may comprise spacers, duplex regions, affinity sequences, intron segments, exon segments, and/or various untranslated elements. These sequences within the enhanced intron elements or enhanced exon elements are arranged to optimize circularization or protein expression.

a. Spacer

In some embodiments, a provided polynucleotide (e.g., a DNA template, a precursor RNA polynucleotide, or a circular RNA polynucleotide) comprises one or more spacers. In certain embodiments, the polynucleotide comprises a first (5′) and/or a second (3′) spacer. In some embodiments, the polynucleotide (e.g., DNA template or precursor linear RNA polynucleotide) comprises one or more spacers in the enhanced intron elements. In some embodiments, the polynucleotide (e.g., DNA template, precursor linear RNA polynucleotide, or a circular RNA polynucleotide) comprises one or more spacers in the enhanced exon elements. In certain embodiments, the polynucleotide comprises a spacer in the 3′ enhanced intron segment and a spacer in the 5′ enhanced intron segment. In certain embodiments, the polynucleotide comprises a spacer in the 3′ enhanced exon segment and another spacer in the 5′ enhanced exon segment to aid with circularization or protein expression due to symmetry created in the overall sequence.

In some embodiments, including a spacer between the 3′ group I intron segment and the core functional element may conserve secondary structures in those regions by preventing them from interacting, thus increasing splicing efficiency. In some embodiments, the first (between 3′ group I intron segment and core functional element) and second (between the two expression sequences and core functional element) spacers comprise additional base pairing regions that are predicted to base pair with each other and not to the first and second duplex regions. In other embodiments, the first (between 3′ group I intron segment and core functional element) and second (between the one of the core functional element and 5′ group I intron segment) spacers comprise additional base pairing regions that are predicted to base pair with each other and not to the first and second duplex regions. In some embodiments, such spacer base pairing brings the group I intron segments in close proximity to each other, further increasing splicing efficiency. Additionally, in some embodiments, the combination of base pairing between the first and second duplex regions, and separately, base pairing between the first and second spacers, promotes the formation of a splicing bubble containing the group I intron segments flanked by adjacent regions of base pairing. Typical spacers are contiguous sequences with one or more of the following qualities: 1) predicted to avoid interfering with proximal structures, for example, the IRES, expression sequence, aptamer, or intron; 2) is at least 7 nt long and no longer than 100 nt; 3) is located after and adjacent to the 3′ intron segment and/or before and adjacent to the 5′ intron segment; and 4) contains one or more of the following: a) an unstructured region at least 5 nt long, b) a region of base pairing at least 5 nt long to a distal sequence, including another spacer, and c) a structured region at least 7 nt long limited in scope to the sequence of the spacer. Spacers may have several regions, including an unstructured region, a base pairing region, a hairpin/structured region, and combinations thereof. In an embodiment, the spacer has a structured region with high GC content. In an embodiment, a region within a spacer base pairs with another region within the same spacer. In an embodiment, a region within a spacer base pairs with a region within another spacer. In an embodiment, a spacer comprises one or more hairpin structures. In an embodiment, a spacer comprises one or more hairpin structures with a stem of 4 to 12 nucleotides and a loop of 2 to 10 nucleotides. In an embodiment, there is an additional spacer between the 3′ group I intron segment and the core functional element. In an embodiment, this additional spacer prevents the structured regions of the IRES or aptamer of a TIE from interfering with the folding of the 3′ group I intron segment or reduces the extent to which this occurs. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 5 and 50, 10 and 50, 20 and 50, 20 and 40, and/or 25 and 35 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyAC sequence. In one embodiment, a spacer comprises about 10%, 20%, 30%, 40%, 50%, 60%, 70%, 80%, 90%, or 100% polyAC content. In one embodiment, a spacer comprises about 10%, 20%, 30%, 40%, 50%, 60%, 70%, 80%, 90%, or 100% polypyrimidine (C/T or C/U) content.

b. Duplex Region

In some embodiments, a provided polynucleotide (e.g., a DNA template, a precursor linear RNA polynucleotide, or a circular RNA polynucleotide provided herein comprise one or more duplex regions. In some embodiments, the polynucleotide comprises a first (5′) duplex region and a second (3′) duplex region. In certain embodiments, the polynucleotide comprises a 5′ external duplex region located within the 3′ enhanced intron segment and a 3′ external duplex region located within the 5′ enhanced intron segment. In some embodiments, the polynucleotide comprise a 5′ internal duplex region located within the 3′ enhanced exon segment and a 3′ internal duplex region located within the 5′ enhanced exon segment. In some embodiments, the polynucleotide comprises a 5′ external duplex region, 5′ internal duplex region, a 3′ internal duplex region, and a 3′ external duplex region.

In certain embodiments, the first and second duplex regions may form perfect or imperfect duplexes. Thus, in certain embodiments at least 75%, 80%, 85%, 90%, 91%, 92%, 93%, 94%, 95%, 96%, 97%, 98%, 99% or 100% of the first and second duplex regions may be base paired with one another. In some embodiments, the duplex regions are predicted to have less than 50% (e.g., less than 45%, less than 40%, less than 35%, less than 30%, less than 25%) base pairing with unintended sequences in the RNA (e.g., non-duplex region sequences). In some embodiments, including such duplex regions on the ends of the precursor RNA strand, and adjacent or very close to the group I intron segment, bring the group I intron segments in close proximity to each other, increasing splicing efficiency. In some embodiments, the duplex regions are 3 to 100 nucleotides in length (e.g., 3-75 nucleotides in length, 3-50 nucleotides in length, 20-50 nucleotides in length, 35-50 nucleotides in length, 5-25 nucleotides in length, 9-19 nucleotides in length). In some embodiments, the duplex regions are about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the duplex regions have a length of about 9 to about 50 nucleotides. In one embodiment, the duplex regions have a length of about 9 to about 19 nucleotides. In some embodiments, the duplex regions have a length of about 20 to about 40 nucleotides. In certain embodiments, the duplex regions have a length of about 30 nucleotides.

In other embodiments, the polynucleotide does not comprise of any duplex regions to optimize translation or circularization.

c. Affinity Sequence

As provided herein, a provided polynucleotide (e.g., a DNA template, a precursor linear RNA polynucleotide, or a circular RNA polynucleotide) may comprise an affinity sequence (or affinity tag). In some embodiments, the affinity tag is located in the 3′ enhanced intron element. In some embodiments, the affinity tag is located in the 5′ enhanced intron element. In some embodiments, both (3′ and 5′) enhanced intron elements each comprise an affinity tag. In one embodiment, an affinity tag of the 3′ enhanced intron element is the length as an affinity tag in the 5′ enhanced intron element. In some embodiments, an affinity tag of the 3′ enhanced intron element is the same sequence as an affinity tag in the 5′ enhanced intron element. In some embodiments, the affinity sequence is placed to optimize oligo-dT purification.

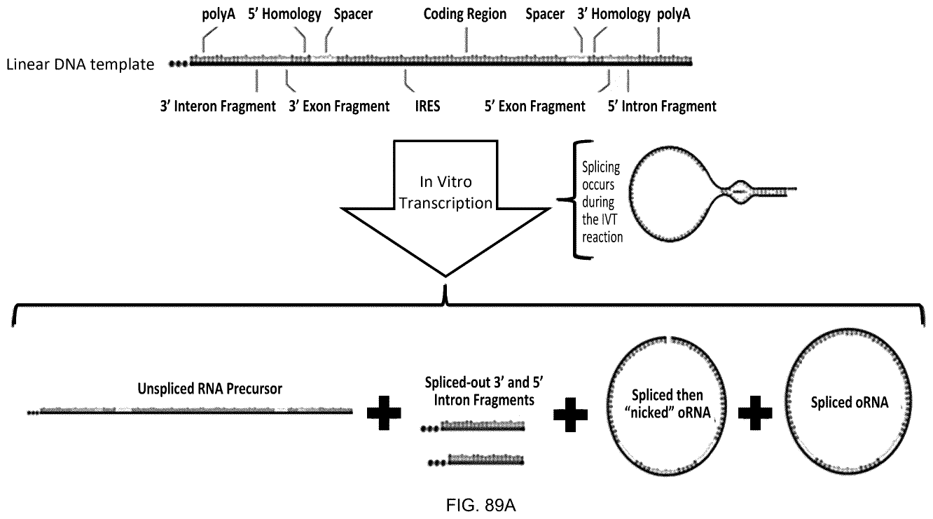

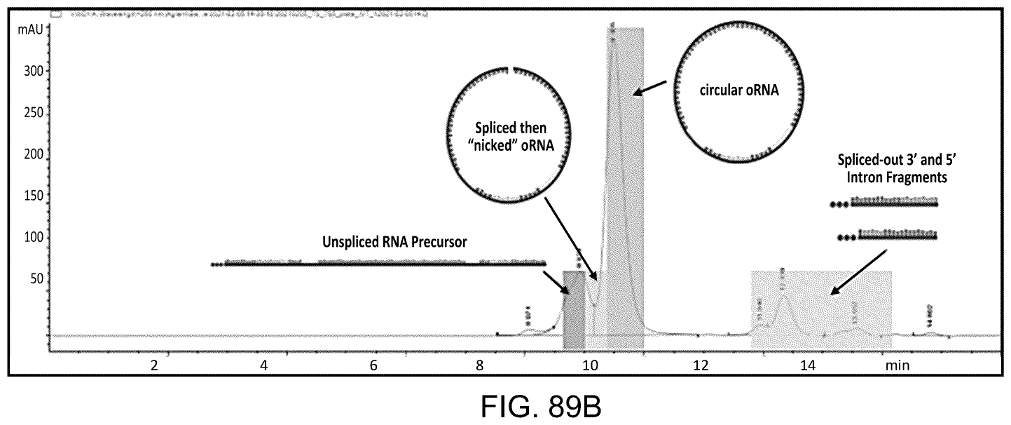

In some embodiments, the one or more affinity tags present in a precursor linear RNA polynucleotide are removed upon circularization. See, for example, FIG. 89A and FIG. 89B. In some embodiments, affinity tags are added to remaining linear RNA after circularization of RNA is performed. In some such embodiments, the affinity tags are added enzymatically to linear RNA. The presence of one or more affinity tags in linear RNA and their absence from circular RNA can facilitate purification of circular RNA. In some embodiments, such purification is performed using a negative selection or affinity-purification method. In some embodiments, such purification is performed using a binding agent that preferentially or specifically binds to the affinity tag.

In some embodiments, an affinity tag comprises a polyA sequence. In some embodiments the polyA sequence is at least 15, 30, or 60 nucleotides long. In some embodiments, the affinity tag comprising a polyA sequence is present in two places in a precursor linear RNA. In some embodiments, one or both polyA sequences are 15-50 nucleotides long. In some embodiments, one or both polyA sequences are 20-25 nucleotides long. In some embodiments, the polyA sequence(s) is removed upon circularization. Thus, an oligonucleotide hybridizing with the polyA sequence, such as a deoxythymidine oligonucleotide (oligo(dT)) conjugated to a solid surface (e.g., a resin), can be used to separate circular RNA from its precursor RNA.

In some embodiments, an affinity tag comprises a sequence that is absent from the circular RNA product. In some such embodiments, the sequence that is absent from the circular RNA product is a dedicated binding site (DBS). In some embodiments, the DBS is an unstructured sequence, i.e., a sequence that does not form a defined structural element, such as a hairpin loop, contiguous dsRNA region, or triple helix. In some embodiments, the DBS sequence forms a random coil. In some embodiments, the DBS comprises at least 25% GC content, at least 50% GC content, at least 75% GC content, or at least 100% GC content. In some embodiments, the DBS comprises at least 25% AC content, at least 50% AC content, at least 75% AC content, or 100% AC content. In some embodiments, the DBS is at least 15, 30, or 60 nucleotides long. In some embodiments, the affinity tag comprising a DBS is present in two places in a precursor linear RNA. In some embodiments, the DBS sequences are each independently 15-50 nucleotides long. In some embodiments, the DBS sequences are each independently 20-25 nucleotides long.

In some embodiments, the DBS sequence(s) is removed upon circularization. Thus, binding agents comprising oligonucleotides comprising a sequence that is complementary to the DBS can be used to facilitate purification of circular RNA. For example, the binding agent may comprise an oligonucleotide complementary to a DBS conjugated to a solid surface (e.g., a resin).

In some embodiments, an affinity sequence or other type of affinity handle, such as biotin, is added to linear RNA by ligation. In some embodiments, an oligonucleotide comprising an affinity sequence is ligated to the linear RNA. In some embodiments, an oligonucleotide conjugated to an affinity handle is ligated to the linear RNA. In some embodiments, a solution comprising the linear RNA ligated to the affinity sequence or handle and the circular RNA that does not comprise an affinity sequence or handle are contacted with a binding agent comprising a solid support conjugated to an oligonucleotide complementary to the affinity sequence or to a binding partner of the affinity handle, such that the linear RNA binds to the binding agent, and the circular RNA is eluted or separated from the solid support.

Any purification method for circular RNA described herein may comprise one or more buffer exchange steps. In some embodiments, buffer exchange is performed after in vitro transcription (IVT) and before additional purification steps. In some such embodiments, the IVT reaction solution is buffer exchanged into a buffer comprising Tris. In some embodiments, the IVT reaction solution is buffer exchanged into a buffer comprising greater than 1 mM or greater than 10 mM one or more monovalent salts, such as NaCl or KCl, and optionally comprising EDTA. In some embodiments, buffer exchange is performed after purification of circular RNA is complete. In some embodiments, buffer exchange is performed after IVT and after purification of circular RNA. In some embodiments, the buffer exchange that is performed after purification of circular RNA comprises exchange of the circular RNA into water or storage buffer. In some embodiments, the storage buffer comprises 1 mM sodium citrate, pH 6.5.

In certain embodiments, the 3′ enhanced intron element comprises a leading untranslated sequence. In some embodiments, the leading untranslated sequence is a the 5′ end of the 3′ enhanced intron segment. In some embodiments, the leading untranslated sequence comprises of the last nucleotide of a transcription start site (TSS). In some embodiments, the TSS is chosen from a viral, bacterial, or eukaryotic DNA template. In one embodiment, the leading untranslated sequence comprise the last nucleotide of a TSS and 0 to 100 additional nucleotides. In some embodiments, the TSS is a terminal spacer. In one embodiment, the leading untranslated sequence contains a guanosine at the 5′ end upon translation of an RNA T7 polymerase.

In certain embodiments, the 5′ enhanced intron element comprises a trailing untranslated sequence. In some embodiments, the 5′ trailing untranslated sequence is located at the 3′ end of the 5′ enhanced intron element. In some embodiments, the trailing untranslated sequence is a partial restriction digest sequence. In one embodiment, the trailing untranslated sequence is in whole or in part a restriction digest site used to linearize the DNA template. In some embodiments, the restriction digest site is in whole or in part from a natural viral, bacterial or eukaryotic DNA template. In some embodiments, the trailing untranslated sequence is a terminal restriction site fragment.

### d. Enhanced Intron Segments

In some embodiments, the 3′ enhanced intron element and 5′ enhanced intron element each comprise an intron segment. In certain embodiments, a 3′ intron segment is a contiguous sequence at least 75% homologous (e.g., at least 80%, 85%, 90%, 91%, 92%, 93%, 94%, 95%, 96%, 97%, 98%, 99% or 100% homologous) to a 3′ proximal fragment of a natural group I intron including the 3′ splice site dinucleotide. Typically, a 5′ intron segment is a contiguous sequence at least 75% homologous (e.g., at least 80%, 85%, 90%, 91%, 92%, 93%, 94%, 95%, 96%, 97%, 98%, 99% or 100% homologous) to a 5′ proximal fragment of a natural group I intron including the 5′ splice site dinucleotide. In some embodiments, the 3′ intron segment includes the first nucleotide of a 3′ group I splice site dinucleotide. In some embodiments, the 5′ intron segment includes the first nucleotide of a 5′ group I splice site dinucleotide. In other embodiments, the 3′ intron segment includes the first and second nucleotides of a 3′ group I intron segment splice site dinucleotide; and the 5′ intron segment includes the first and second nucleotides of a 3′ group I intron segment dinucleotide.

### E. Enhanced Exon Segments

In certain embodiments, a provided polynucleotide (e.g., a DNA template, a linear precursor RNA polynucleotide, or a circular RNA polynucleotide) comprises an enhanced exon segment. In some embodiments, following a 5′ to 3′ order, the 3′ enhanced exon element is located upstream to core functional element. In some embodiments, following a 5′ to 3′ order, the 5′ enhanced intron element is located downstream to the core functional element.

According to the present invention, the 3′ enhanced exon element and 5′ enhanced exon element each comprise an exon segment. In some embodiments, the 3′ enhanced exon element comprises a 3′ exon segment. In some embodiments, the 5′ enhanced exon element comprises a 5′ exon segment. In certain embodiments, as provided herein, the 3′ exon segment and 5′ exon segment each comprises a group I intron segment and 1 to 100 nucleotides of an exon sequence. In certain embodiments, a 3′ intron segment is a contiguous sequence at least 75% homologous (e.g., at least 80%, 85%, 90%, 91%, 92%, 93%, 94%, 95%, 96%, 97%, 98%, 99% or 100% homologous) to a 3′ proximal fragment of a natural group I intron including the 3′ splice site dinucleotide. Typically, a 5′ group I intron segment is a contiguous sequence at least 75% homologous (e.g., at least 80%, 85%, 90%, 91%, 92%, 93%, 94%, 95%, 96%, 97%, 98%, 99% or 100% homologous) to a 5′ proximal fragment of a natural group I intron including the 5′ splice site dinucleotide. In some embodiments, the 3′ exon segment comprises a second nucleotide of a 3′ group I intron splice site dinucleotide and 1 to 100 nucleotides of an exon sequence. In some embodiments, the 5′ exon segment comprises the first nucleotide of a 5′ group I intron splice site dinucleotide and 1 to 100 nucleotides of an exon sequence. In some embodiments, the exon sequence comprises in part or in whole from a naturally occurring exon sequence from a virus, bacterium or eukaryotic DNA vector. In other embodiments, the exon sequence further comprises a synthetic, genetically modified (e.g., containing modified nucleotide), or other engineered exon sequence.

In one embodiment, where the 3′ intron segment comprises both nucleotides of a 3′ group I splice site dinucleotide and the 5′ intron segment comprises both nucleotides of a 5′ group I splice site dinucleotide, the exon segments located within the 5′ enhanced exon element and 3′ enhanced exon element does not comprise of a group I splice site dinucleotide.

f. Exemplary Permutation of the Enhanced Intron Elements & Enhanced Exon Elements

For means of example and not intended to be limiting, in some embodiments, a 3′ enhanced intron element comprises in the following 5′ to 3′ order: a leading untranslated sequence, a 5′ affinity tag, an optional 5′ external duplex region, a 5′ external spacer, and a 3′ intron segment. In same embodiments, the 3′ enhanced exon element comprises in the following 5′ to 3′ order: a 3′ exon segment, an optional 5′ internal duplex region, an optional 5′ internal duplex region, and a 5′ internal spacer. In the same embodiments, the 5′ enhanced exon element comprises in the following 5′ to 3′ order: a 3′ internal spacer, an optional 3′ internal duplex region, and a 5′ exon segment. In still the same embodiments, the 3′ enhanced intron element comprises in the following 5′ to 3′ order: a 5′ intron segment, a 3′ external spacer, an optional 3′ external duplex region, a 3′ affinity tag, and a trailing untranslated sequence.

### B. Core Functional Element

In some embodiments, a provided polynucleotide (e.g., a DNA template, a linear precursor RNA polynucleotide, or a circular RNA polynucleotide) comprises a core functional element. In some embodiments, the core functional element comprises a coding or noncoding element. In certain embodiments, the core functional element may contain both a coding and noncoding element. In some embodiments, the core functional element further comprises translation initiation element (TIE) upstream to the coding or noncoding element. In some embodiments, the core functional element comprises a termination element. In some embodiments, the termination element is located downstream to the TIE and coding element. In some embodiments, the termination element is located downstream to the coding element but upstream to the TIE. In certain embodiments, where the coding element comprises a noncoding region, a core functional element lacks a TIE and/or a termination element.

a. Coding or Noncoding Element

In some embodiments, the polynucleotides provided herein comprise a coding or noncoding element or a combination of both. In some embodiments, the coding element comprises an expression sequence. In some embodiments, the coding element encodes at least one therapeutic protein.

In some embodiments, a provided circular RNA encodes two or more polypeptides. In some embodiments, the circular RNA is a bicistronic RNA. The sequences encoding the two or more polypeptides can be separated by a ribosomal skipping element or a nucleotide sequence encoding a protease cleavage site. In certain embodiments, the ribosomal skipping element encodes thosea-asigna virus 2A peptide (T2A), porcine teschovirus-1 2 A peptide (P2A), foot-and-mouth disease virus 2A peptide (F2A), equine rhinitis A vims 2A peptide (E2A), cytoplasmic polyhedrosis vims 2A peptide (BmCPV 2A), or flacherie vims of B. mori 2A peptide (BmIFV 2A).

b. Translation Initiation Element (TIE)

As provided herein in some embodiments, the core functional element comprises at least one translation initiation element (TIE). TIEs are designed to allow translation efficiency of an encoded protein. Thus, optimal core functional elements comprising only of noncoding elements lack any TIEs. In some embodiments, core functional elements comprising one or more coding element will further comprise one or more TIEs.

In some embodiments, the TIE comprises a naturally occurring sequence and/or a synthetic, non-naturally occurring sequence capable of promoting and/or initiating translation of an encoded protein.

In some embodiments, a TIE comprises an untranslated region (UTR). In certain embodiments, the TIE provided herein comprise an internal ribosome entry site (IRES). Inclusion of an IRES permits the translation of one or more open reading frames from a circular RNA (e.g., open reading frames that form the expression sequences). The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20: 102-110; Kobayashi et al., BioTechniques (1996) 21:399-402; and Mosser et al., BioTechniques 1997 22 150-161.

i. Natural Ties: Viral, & Eukaryotic/Cellular Internal Ribosome Entry Site (IRES)

A multitude of IRES sequences are available and include sequences derived from a wide variety of viruses, such as from leader sequences of picornaviruses such as the encephalomyocarditis virus (EMCV) UTR (Jang et al., J. Virol. (1989) 63: 1651-1660), the polio leader sequence, the hepatitis A virus leader, the hepatitis C virus IRES, human rhinovirus type 2 IRES (Dobrikova et al., Proc. Natl. Acad. Sci. (2003) 100(25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like.

Different IRES sequences have varying ability to drive protein expression, and the ability of any particular identified or predicted IRES sequence to drive protein expression from linear mRNA or circular RNA constructs is unknown and unpredictable. In certain embodiments, potential IRES sequences can be bioinformatically identified based on sequence positions in viral sequences. However, the activity of such sequences has been previously uncharacterized. As demonstrated herein, such IRES sequences may have differing protein expression capability depending on cell type, for example in T cells, liver cells, or muscle cells. In some embodiments, the novel IRES sequences described herein may have at least 2, 3, 4, 5, 6, 7, 8, 9, 10, 20, 50, or 100 fold increased expression in a particular cell type compared to previously described EMCV IRES sequences.

In some embodiments, for driving protein expression, a provided circular RNA comprises an IRES operably linked to a protein coding sequence.

In some embodiments, a TIE comprises a sequence from a nonviral untranslated region (UTR). In certain embodiments, a TIE comprises a sequence from a mammalian UTR and is capable of driving protein expression when operably linked to the coding sequence for said protein. In certain embodiments, a TIE comprises a sequence from a primate UTR and is capable of driving protein expression when operably linked to the coding sequence for said protein. In certain embodiments, a TIE comprises a sequence from a human UTR and is capable of driving protein expression when operably linked to the coding sequence for said protein. In certain embodiments, a TIE comprises a sequence from an invertebrate UTR and is capable of driving protein expression when operably linked to the coding sequence for said protein. In certain embodiments, a TIE comprises a sequence from a drosophila UTR and is capable of driving protein expression when operably linked to the coding sequence for said protein.

In some embodiments, the IRES comprises a sequence of any one of the IRES sequences SEQ ID NOS: 14067-24829 or a fragment thereof. In some embodiments, the IRES comprises a sequence at least 80%, 85%, 90%, 91%, 92%, 93%, 94%, 95%, 96%, 97%, 98%, or 99% identical to a sequence of any one of the IRES sequences SEQ ID NOS: 14067-24829. In some embodiments, the circular RNA disclosed herein comprises an IRES sequence at least 80%, 85%, 90%, 91%, 92%, 93%, 94%, 95%, 96%, 97%, 98%, or 99% identical to a sequence of any one of the IRES sequences SEQ ID NOS: 14067-24829. In some embodiments, the circular RNA disclosed herein comprises an IRES sequence of any one of the IRES sequences SEQ ID NOS: 14067-24829 or a fragment thereof.

In some embodiments, the IRES comprises a sequence set forth in any one of SEQ ID NOs: 793, 876, 1017, 1216, and 3291 or a fragment thereof. In some embodiments, the IRES comprises a sequence at least 80%, 85%, 90%, 91%, 92%, 93%, 94%, 95%, 96%, 97%, 98%, or 99% identical to a sequence set forth in any one of SEQ ID NOs: 793, 876, 1017, 1216, and 3291. In some embodiments, the circular RNA disclosed herein comprises an IRES sequence at least 80%, 85%, 90%, 91%, 92%, 93%, 94%, 95%, 96%, 97%, 98%, or 99% identical to set forth in any one of SEQ ID NOs: 793, 876, 1017, 1216, and 3291. In some embodiments, the circular RNA disclosed herein comprises an IRES sequence set forth in any one of SEQ ID NOs: 793, 876, 1017, 1216, and 3291 or a fragment thereof. In some embodiments, the IRES comprises a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302 or a fragment thereof. In some embodiments, the IRES comprises a sequence at least 80%, 85%, 90%, 91%, 92%, 93%, 94%, 95%, 96%, 97%, 98%, or 99% identical to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In some embodiments, the circular RNA disclosed herein comprises an IRES sequence at least 80%, 85%, 90%, 91%, 92%, 93%, 94%, 95%, 96%, 97%, 98%, or 99% identical to set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In some embodiments, the circular RNA disclosed herein comprises an IRES sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302 or a fragment thereof. In some embodiments, the IRES comprises a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301 or a fragment thereof. In some embodiments, the IRES comprises a sequence at least 80%, 85%, 90%, 91%, 92%, 93%, 94%, 95%, 96%, 97%, 98%, or 99% identical to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In some embodiments, the circular RNA disclosed herein comprises an IRES sequence at least 80%, 85%, 90%, 91%, 92%, 93%, 94%, 95%, 96%, 97%, 98%, or 99% identical to set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301. In some embodiments, the circular RNA disclosed herein comprises an IRES sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301 or a fragment thereof.

In some embodiments, the IRES comprises a sequence selected from SEQ ID NOs: 1-2983 and 3282-3287 or a fragment thereof. In some embodiments, the IRES comprises a sequence at least 80%, 85%, 90%, 91%, 92%, 93%, 94%, 95%, 96%, 97%, 98%, or 99% identical to a sequence selected from SEQ ID NOs: 1-2983 and 3282-3287. In some embodiments, the circular RNA disclosed herein comprises an IRES sequence at least 80%, 85%, 90%, 91%, 92%, 93%, 94%, 95%, 96%, 97%, 98%, or 99% identical to a sequence selected from SEQ ID NOs: 1-2983 and 3282-3287. In some embodiments, the circular RNA disclosed herein comprises an IRES sequence selected from SEQ ID NOs: 1-2983 and 3282-3287 or a fragment thereof. Modifications of IRES and accessory sequences are disclosed herein to increase or reduce IRES activities, for example, by truncating the 5′ and/or 3′ ends of the IRES, adding a spacer 5′ to the IRES, modifying the 6 nucleotides 5′ to the translation initiation site (Kozak sequence), modification of alternative translation initiation sites, and creating chimeric/hybrid IRES sequences. In some embodiments, the IRES sequence in the circular RNA disclosed herein comprises one or more of these modifications relative to a native IRES (e.g., SEQ ID NOs: 1-2983 and 3282-3287). In some embodiments, the IRES comprises one or more modifications in any one of SEQ ID NOs: 793, 876, 1017, 1216, and 3291. In some embodiments, the IRES comprises one or more modifications in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302. In some embodiments, the IRES comprises one or more modifications in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

In some embodiments, the IRES is an Aalivirus, Ailurivirus, Ampivirus, Anativirus, Aphthovirus, Aquamavirus, Avihepatovirus, Avisivirus, Boosepivirus, Bopivirus, Caecilivirus, Cardiovirus, Cosavirus, Crahelivirus, Crohivirus, Danipivirus, Dicipivirus, Diresapivirus, Enterovirus, Erbovirus, Felipivirus, Fipivirus, Gallivirus, Gruhelivirus, Grusopivirus, Harkavirus, Hemipivirus, Hepatovirus, Hunnivirus, Kobuvirus, Kunsagivirus, Limnipivirus, Livupivirus, Ludopivirus, Malagasivirus, Marsupivirus, Megrivirus, Mischivirus, Mosavirus, Mupivirus, Myrropivirus, Orivirus, Oscivirus, Parabovirus, Parechovirus, Pasivirus, Passerivirus, Pemapivirus, Poecivirus, Potamipivirus, Pygoscepivirus, Rabovirus, Rafivirus, Rajidapivirus, Rohelivirus, Rosavirus, Sakobuvirus, Salivirus, Sapelovirus, Senecavirus, Shanbavirus, Sicinivirus, Symapivirus, Teschovirus, Torchivirus, Tottorivirus, Tremovirus, Tropivirus, Hepacivirus, Pegivirus, Pestivirus, Flavivirus IRES.

In some embodiments, the IRES is an IRES sequence of Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAPI, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the IRES comprises in whole or in part from a eukaryotic or cellular IRES. In certain embodiments, the IRES is from a human gene, where the human gene is ABCF1, ABCG1, ACAD10, ACOT7, ACSS3, ACTG2, ADCYAP1, ADK, AGTR1, AHCYL2, AHI1, AKAP8L, AKR1A1, ALDH3A1, ALDOA, ALG13, AMMECRIL, ANGPTL4, ANK3, AOC3, AP4B1, AP4E1, APAF1, APBB1, APC, APH1A, APOBEC3D, APOM, APP, AQP4, ARHGAP36, ARL13B, ARMC8, ARMCX6, ARPC1A, ARPC2, ARRDC3, ASAP1, ASB3, ASB5, ASCL1, ASMTL, ATF2, ATF3, ATG4A, ATP5B, ATP6V0A1, ATXN3, AURKA, AURKA, AURKA, AURKA, B3GALNT1, B3GNTL1, B4GALT3, BAAT, BAG1, BAIAP2, BAIAP2L2, BAZ2A, BBX, BCAR1, BCL2, BCS1L, BET1, BID, BIRC2, BPGM, BPIFA2, BRINP2, BSG, BTN3A2, C12orf43, C14orf93, C17orf62, C1orf226, C21orf62, C2orf15, C4BPB, C4orf22, C9orf84, CACNA1A, CALCOCO2, CAPN11, CASP12, CASP8AP2, CAV1, CBX5, CCDCl20, CCDCl7, CCDCl86, CCDC51, CCN1, CCND1, CCNT1, CD2BP2, CD9, CDC25C, CDC42, CDC7, CDCA7L, CDIP1, CDK1, CDK11A, CDKN1B, CEACAM7, CEP295NL, CFLAR, CHCHD7, CHIA, CHIC1, CHMP2A, CHRNA2, CLCN3, CLEC12A, CLEC7A, CLECL1, CLRN1, CMSS1, CNIH1, CNR1, CNTN5, COG4, COMMD1, COMMD5, CPEB1, CPS1, CRACR2B, CRBN, CREM, CRYBG1, CSDE1, CSF2RA, CSNK2A1, CSTF3, CTCFL, CTH, CTNNA3, CTNNB1, CTNNB1, CTNND1, CTSL, CUTA, CXCR5, CYB5R3, CYP24A1, CYP3A5, DAG1, DAP3, DAP5, DAXX, DCAF4, DCAF7, DCLRElA, DCP1A, DCTN1, DCTN2, DDX19B, DDX46, DEFB123, DGKA, DGKD, DHRS4, DHX15, D103, DLG1, DLL4, DMD UTR, DMD ex5, DMKN, DNAH6, DNAL4, DUSP13, DUSP19, DYNC1I2, DYNLRB2, DYRKIA, ECI2, ECT2, EIFIAD, EIF2B4, EIF4G1, EIF4G2, EIF4G3, ELANE, ELOVL6, ELP5, EMCN, ENOl, EPB41, ERMN, ERVV-1, ESRRG, ETFB, ETFBKMT, ETV1, ETV4, EXD1, EXT1, EZH2, FAM111B, FAM157A, FAM213A, FBXO25, FBXO9, FBXW7, FCMR, FGF1, FGF1, FGF1A, FGF2, FGF2, FGF-9, FHL5, FMR1, FN1, FOXP1, FTH1, FUBP1, G3BP1, GABBR1, GALC, GART, GAS7, gastrin, GATA1, GATA4, GFM2, GHR, GJB2, GLIl, GLRA2, GMNN, GPAT3, GPATCH3, GPR137, GPR34, GPR55, GPR89A, GPRASP1, GRAP2, GSDMB, GSTO2, GTF2B, GTF2H4, GUCYlB2, HAX1, HCST, HIGD1A, HIGD1B, HIPK1, HISTIHIC, HIST1H3H, HK11, HLA-DRB4, HMBS, HMGA1, HNRNPC, HOPX, HOXA2, HOXA3, HPCAL1, HR, HSP90AB1, HSPA1A, HSPA4L, HSPA5, HYPK, IFFO1, IFT74, IFT81, IGF1, IGF1R, IGF1R, IGF2, IL11, IL17RE, IL1RL1, IL1RN, IL32, IL6, ILF2, ILVBL, INSR, INTS13, IP6K1, ITGA4, ITGAE, KCNE4, KERA, KIAA0355, KIAA0895L, KIAA1324, KIAA1522, KIAA1683, KIF2C, KIZ, KLHL31, KLK7, KRR1, KRT14, KRT17, KRT33A, KRT6A, KRTAP10-2, KRTAP13-3, KRTAP13-4, KRTAP5-11, KRTCAP2, LACRT, LAMB1, LAMB3, LANCL1, LBX2, LCAT, LDHA, LDHAL6A, LEF1, LINC-PINT, LMO3, LRRC4C, LRRC7, LRTOMT, LSM5, LTB4R, LYRM1, LYRM2, MAGEA11, MAGEA8, MAGEB1, MAGEB16, MAGEB3, MAPT, MARS, MC1R, MCCC1, METTL12, METTL7A, MGC16025, MGC16025, MIA2, MIA2, MITF, MKLN1, MNT, MORF4L2, MPD6, MRFAP1, MRPL21, MRPS12, MSI2, MSLN, MSN, MT2A, MTFR1L, MTMR2, MTRR, MTUS1, MYB, MYC, MYCL, MYCN, MYL10, MYL3, MYLK, MYO1A, MYT2, MZB1, NAP1L1, NAV1, NBAS, NCF2, NDRG1, NDST2, NDUFA7, NDUFB11, NDUFC1, NDUFSI, NEDD4L, NFAT5, NFE2L2, NFE2L2, NFIA, NHEJ1, NHP2, NIT1, NKRF, NME1-NME2, NPAT, NR3C1, NRBF2, NRF1, NTRK2, NUDCD1, NXF2, NXT2, ODC1, ODF2, OPTN, OR10R2, OR11L1, OR2M2, OR2M3, OR2M5, OR2T10, OR4C15, OR4F17, OR4F5, OR5H1, OR5K1, OR6C3, OR6C75, OR6N1, OR7G2, p53, P2RY4, PAN2, PAQR6, PARP4, PARP9, PC, PCBP4, PCDHGC3, PCLAF, PDGFB, PDZRN4, PELO, PEMT, PEX2, PFKM, PGBD4, PGLYRP3, PHLDA2, PHTF1, PI4 KB, PIGC, PIM1, PKD2L1, PKM, PLCB4, PLD3, PLEKHA1, PLEKHBI, PLS3, PML, PNMA5, PNN, POC1A, POC1B, POLD2, POLD4, POU5F1, PPIG, PQBP1, PRAME, PRPF4, PRR11, PRRT1, PRSS8, PSMA2, PSMA3, PSMA4, PSMD11, PSMD4, PSMD6, PSME3, PSMG3, PTBP3, PTCH1, PTHLH, PTPRD, PUS7L, PVRIG, QPRT, RAB27A, RAB7B, RABGGTB, RAETIE, RALGDS, RALYL, RARB, RCVRN, REG3G, RFC5, RGL4, RGS19, RGS3, RHD, RINL, RIPOR2, RITA1, RMDN2, RNASE1, RNASE4, RNF4, RPA2, RPL17, RPL21, RPL26L1, RPL28, RPL29, RPL41, RPL9, RPS11, RPS13, RPS14, RRBP1, RSU1, RTP2, RUNX1, RUNX1T1, RUNX1T1, RUNX2, RUSC1, RXRG, S100A13, S100A4, SAT1, SCHIPI, SCMH1, SEC14L1, SEMA4A, SERPINA1, SERPINB4, SERTAD3, SFTPD, SH3D19, SHC1, SHMT1, SHPRH, SIM1, SIRT5, SLC11A2, SLC12A4, SLC16A1, SLC25A3, SLC26A9, SLC5A11, SLC6A12, SLC6A19, SLC7A1, SLFN11, SLIRP, SMAD5, SMARCADI, SMN1, SNCA, SNRNP200, SNRPB2, SNX12, SOD1, SOX13, SOX5, SP8, SPARCL1, SPATA12, SPATA31C2, SPN, SPOP, SQSTM1, SRBD1, SRC, SREBF1, SRPK2, SSB, SSB, SSBP1, ST3GAL6, STAB1, STAMBP, STAU1, STAU1, STAU1, STAU1, STAU1, STK16, STK24, STK38, STMN1, STX7, SULT2B1, SYK, SYNPR, TAF1C, TAGLN, TANK, TAS2R40, TBC1D15, TBXAS1, TCF4, TDGF1, TDP2, TDRD3, TDRD5, TESK2, THAP6, THBD, THTPA, TIAM2, TKFC, TKTL1, TLR10, TM9SF2, TMC6, TMCO2, TMED10, TMEM116, TMEM126A, TMEM159, TMEM208, TMEM230, TMEM67, TMPRSS13, TMUB2, TNFSF4, TNIP3, TP53, TP53, TP73, TRAF1, TRAK1, TRIM31, TRIM6, TRMT1, TRMT2B, TRPM7, TRPM8, TSPEAR, TTC39B, TTLL11, TUBB6, TXLNB, TXNIP, TXNL1, TXNRD1, TYROBP, U2AF1, UBA1, UBE2D3, UBE2I, UBE2L3, UBE2V1, UBE2V2, UMPS, UNG, UPP2, USMG5, USP18, UTP14A, UTRN, UTS2, VDR, VEGFA, VEGFA, VEPH1, VIPAS39, VPS29, VSIG10L, WDHD1, WDR12, WDR4, WDR45, WDYHV1, WRAP53, XIAP, XPNPEP3, YAP1, YWHAZ, YY1AP1, ZBTB32, ZNF146, ZNF250, ZNF385A, ZNF408, ZNF410, ZNF423, ZNF43, ZNF502, ZNF512, ZNF513, ZNF580, ZNF609, ZNF707, or ZNRD1.

ii. Synthetic Ties: Aptamer Complexes, Modified Nucleotides, IRES Variants & Other Engineered TIES

As contemplated herein, in certain embodiments, a translation initiation element (TIE) comprises a synthetic TIE. In some embodiments, a synthetic TIE comprises aptamer complexes, synthetic IRES or other engineered TIES capable of initiating translation of a linear RNA or circular RNA polynucleotide.

In some embodiments, one or more aptamer sequences is capable of binding to a component of a eukaryotic initiation factor to either enhance or initiate translation. In some embodiments, aptamer may be used to enhance translation in vivo and in vitro by promoting specific eukaryotic initiation factors (eIF) (e.g., aptamer in WO 2019/081383 A1 is capable of binding to eukaryotic initiation factor 4F (eIF4F). In some embodiments, the aptamer or a complex of aptamers may be capable of binding to EIF4G, EIF4E, EIF4A, EIF4B, EIF3, EIF2, EIF5, EIF1, EIF1A, 40S ribosome, PCBP1 (polyC binding protein), PCBP2, PCBP3, PCBP4, PABP1 (polyA binding protein), PTB, Argonaute protein family, HNRNPK (heterogeneous nuclear ribonucleoprotein K), or La protein.

iii. TIE Consensus

In some embodiments, a TIE disclosed herein comprises a naturally occurring and/or synthetic sequence and includes an IRES consensus sequence. In some embodiments, the TIE comprises a consensus sequence as set forth in the Table of Exemplary Consensus Sequences, below, wherein N is any nucleotide (e.g., pursuant to IUPAC). In some embodiments, the TIE comprises at least 100 nucleotides, at least 200 nucleotides, at least 300 nucleotides, at least 400 nucleotides, at least 500 nucleotides, at least 600 nucleotides, or at least 700 nucleotides (e.g., contiguous nucleotides) of said consensus sequence. Table of Exemplary Consensus Sequences (Table A) TCNNNNNNANNNNNCNNTTNAAANNNNCTGTGGGTTGTTCCCACNCACA

TABLE A
TABLE of Exemplary Consensus Sequences

TCNNNNNNANNNNNNNNCNNTTNAAANNNNCTGTGGGTTGTTCCCACNCACA
GGGCCCACTGGGCGCTAGCACTCTGATTTNACGAAATCNTTGTGCGCCTGTTTT
ATATCCCTTCCCCCNANTNNGAAACNTAGAAGCAATGCGCANCACTGATCAAT
AGNAGGCGTAACGCGCCAGTTACGTCATGATCAAGCANATCTGTNNCCCCGGA
CNGAGTATCAATAGACTGCTTNCGCGGTTGAAGGAGAAANNGTTCGTTANCCG
GCTAACTACTTCGAGAAGCCCAGTANCACCATGGAAGCTGCAGGGTGTTTCGC
TCAGCACTTCCCCCGTGTAGATCAGGTCGATGAGNCACTGCAANCCCCACAGG
NGANTGTGGCAGTGGCTGCGTTGGCGGCCTGCCTATGGGGAGACCCATAGGAC
GCTCTAATGTGGACATGGTGNGAAGAGCCTATTGAGCTAGTTAGTAGTCCTCC
GGCCCCTGAATGCGGCTAATCCTAACTGCGGAGCACANGCCNTCAACCCAGAG
GGTAGTGTGTCGTAANGGGCAACTCTGCAGCGGAACCGACTACTTTGGGTGTC
CGTGTTTCTTNTTATTCTTATATTGGCNGCTTATGGTGACAATTACAGAATTGTT
ACCATATAGCTATTGGATTGGCCATCCGGTGTGNAATAGAGCTGTTATATACCT
NTTTGTTGGNTTTGTNCCACTNACTTTAAAATCTATANNNACNCTNAANTTNAT
ATTNNCNCTCAANACANTTNAAC
(SEQ ID NO: 24867)
CTAATGTCTGNTNNNNNNNNNNNNNNNNNNNNNCCACTCACAGGACCCACGC
GGTGCTAGCACTCTGGTTCCACGGGACCTTTGTGCGCCTGTTTTATGTCCCCTN
CCCCAATTTGTAACTTAGAAGCAATTTGCAACACTGATCAGTAGCAGGCATAG
CGCACCAGCTATGTCTTGATCAAGCACTTCTGTTTCCCCGGACCGAGTATCAAT
AGACTGTNCACACGGTTGAAGGAGAAAGCGTCCGTTATCCGGCTAACTACTTC
GAGAAACCCAGTAGCACCATTGAAACTACAGAGTGTTTCGCTCCACACTTCCC
CCGTGTAGATCAGGTCGATGAGTCACTGCAATCCCCACGGGCGACCGTGGCAG
TGGCTGCGCTGGCGGCCTGCCTATGGGGCAACCCATAGGACGCTCTAATGTGG
ACATGGTGTGAAGAGTCTATTGAGCTAGTTAGTAGTCCTCCGGCCCCTGAATGC
GGCTAATCCTAACTGCGGAGCACATGCCTTCAATCCAGAGGGTAGTGTGTCGT
AATGGGCAACTCTGCAGCGGAACCGACTACTTTGGGTGTCCGTGTTTCCTTTTA
TTCTCACATTGGCTGCTTATGGTGACAATTACNGAATTGTTACCATATAGCTAT
TGGATTGGCCATCCGGTGTGCAATAGAGCTATTATATACCTATTTGTTGGCTTT
GTACCACTNACCTTAAAATCTNTAACCACTCTCGAGTGTATATTAACCCTTAAT
ACAATCAAAC
(SEQ ID NO: 24868)
NNNNNNNNNNNNNNNNNNNNNTNNNNNNNNNNNNNNNNNNNNNNCACTCAC
AGGACCCACGCGGTGCTAGCACTCTGGTTCCACGGNACCTTTGTGCGCCTGTTT
TANGTCCCCTCTCCCCAATTTGTAACTTAGAAGCAATTTNCAACACTGATCANT
AGCAGGCATNGCGCACCAGCTATGTCTTGATCAAGCACTTCTGTTTCCCCGGAC
CGAGTATCAATAGACTGTTCACACGGTTGAAGGAGAAAGCGTCCGTTATCCGG
CTAACTACTTCGAGAAACCCAGTAGCNCCATTGAAACTNCAGAGTGTTTCGCT
CCACACTTCCCCCGTGTAGATCAGGTCGATGAGTCACTGCAATCCCCACGGGC
GACCGTGGCAGTGGCTGCGCTGGCGGCCTGCCTATGGGGCAACCCATAGGACG
CTCTAATGTGGACATGGTGNGAAGAGTCTATTGAGCTAGTTAGTAGTCCTCCG
GCCCCTGAATGCGGCTAATCCTAACTGCGGAGCACATGCCTTCAANCCAGAGG
GTAGTGTGTCGTAATGGGCAACTCTGCAGCGGAACCGACTACTTTGGGTGTCC
GTGTTTCCTTTTATTCTCACATTGGCTGCTTATGGTGACAATTACAGAATTGTTA
CCATATAGCTATTGGATTGGCCATCCGGTGTGCAATAGAGCTATTATATACCTA
TTTGTTGGCTTTGTACCACTNACCTTAAAATCTNTAACCACTCTNGAGTNTATA
TTAACCCTTAATACAATCAAAC
(SEQ ID NO: 24869)
TATGTTTCCNNNCTNNTNGNNNNNNNNNNNNNNNNGGTNGTTCCCACCCACAG
GGCCCACTGGGCGCCAGTACACTGGTATGCCNNNNCCTNGGTATNACGGTACC
NTTNNNNNNCAGTACCTTTGTACGCCTGTTTTATATNCCCTCCCCNAANNGTAA
CCTTAGAAGTTTAAATAAACAAAGACCAATAGGAGTCCAACANCCAGTTGGAT
TGCGGTCAAGCACTTCTGTCTCCCCGGACCTAGTAGTGATAGGCTGTACCCACG
GCCGAAGATGAACCCGTCCGTTATCCGGCCAGCTACTTCGAGAAGCCTAGTAN
CATCAAAGATCTGTCTTGGCGTTTCGCTCAGCGCGTTCCCCCCGCGTAGATCGG
GCTGATGGGTCTCCGCATNCCCCACGGGCGACCGTGGCGGAGGCCGCGTGGCG
GCCCGCCTATGGCGAAAGCCATAGGACGCCATTTCAGTGACAGGGTGTGAAGA
GCCTATTGAGCTAGTTGGTAGTCCTCCGGCCCCTGAATGCGGCTAATCCTAACC
ACGGAGCNTCCGCCAACAAACCAGTTGGNAGGGNGTCGTAACGGGTAACTCT
GTGGCGGAACCGACTACTTTGGGTGTCCGTGTTTCCTTTTGATCCTATTTTGGCT
GCTTATGGTGACAACGATAAGTTGTTATCATAAAGCTTTTGGGTTGGCCACCTG
GAAAAAGTTATCAGTGTTTGATATTGTTCGGCTTTCACGCCTACCAATAAAACA
AGCTTTATATTTNTTAATTTCATTTTATCCATNNNAGGATATTACTGAATTTCAT
NTACAACTNTTGCTCACANTATCAGCAAN
(SEQ ID NO: 24870)
ANNNNNNNNNNNNNNNNNNNNTTAAAACAGCCTGTGGGTTGCACCCACCCAC
AGGGCCNACTGGGCGCTAGCACTCTGGNACTNAGGTACCTTTGTGCGCCTGTTT
TNNNTNCCCTTNCCCCGAAGTAACTTAGAAGCTGTNAANCAACGATCAANAGN
AGGTGTGACACACCAGTCATANCTTGATCAAGCACTTCTGTTTCCCCGGACTGA
GTATCAATAGGCTGCTCGCGCGGCTGAAGGAGAAAACGTTCGTTACCCGACCA
ACTACTTCGAGAAGCTTAGTACCACCATGAACGAGGCAGNGTGTTTCGNTCAG
CACAACCCCAGTGTAGATCAGGCTGATGAGTCACTGCAACCCCCATGGGCGAC
CATGGCAGTGGCTGCGTTGGCGGCCTGCCCATGGAGAAATCCATGGGACGCTC
TAATTCTGACATGGTGTGAAGAGCCTATTGAGCTAGCTGGTAGTCCTCCGGCCC
CTGAATGCGGCTAATCCTAACTGCGGAGCACATGCTCACAAACCAGTGGGTGG
TGTGTCGTAACGGGCAACTCTGCAGCGGAACCGACTACTTTGGGTGTCCGTGTT
TCCTTTTTATTCTTATNTTGGCTGCTTATGGTGACAATCAAAGANTTGTTACCAT
ATAGCTATTGGATTGGCCATCCGGTGTGCAACAGAGCAATTGTTTACCTATTTN
TTGGTTTTGTACCANTATCACTGAAGTCTGTGATCACTCTCAAATTCATTTTGAC
CCTCAACNCAATCAAAC
(SEQ ID NO: 24871)
ATTTACCATGTGNNNNNNNNNNNNNNNNNNNNNNNNANNNNTGTGCCCAATC
TCTTGACTCCTGCTGGAACCACCGACCAGTAGTGTCCAAAATGCNAGGTGGAA
AATCCTCCCTTCCCCTCTGGGCTNCATGCCCNGCATCCTCCCCCCAGCCTGACG
TGCCACAGGCTGTGCAAAGACCCCGCGAAAGCTGCCAAAAGTGGCAATTGTGG
GTCCCCCCTTTGTCAAGGCGTCGAGTCTTTCTCCCTTAAGGCTAGNCCNGTCAG
TGAACTCTGTCGGGCAACTAGTGACGCCACTGCATGCCTCCGACCTCGGCCGC
GGAGTGCTGCCCCCCAAGTCATGCCCCTGACCACAAGTTGTGCTGTCTGGCAA
ACANTGTCTGTGAGAATGTTCCGCTGTGGCTGCCAAGCCTGGTAACAGGCTGC
CCCAGTGTGCGTANTTCTCATCCAGACNTCGGTCTGGCAACTTGCTGTTAAGAC
ATGGCGTAAGGGGCGTGTGCCAACGCCCTGGAACGAGTGTCCACTCTAATACC
CCGAGGAATGCTACGCAGGTACCCCTGGCTCGCCAGGGATCTGAGCGTAGGCT
AATTGTCTAAGGGTATTTTCATTTCCCACNCTCTNNTTCTTGTTCATA
(SEQ ID NO: 24872)
CAGAACTTGTGTCGTGGATAATGTAACTTAGAAGTTATTAAACAAAGTTCAAT
AGAAGGGGGTACAAACCAGTGCCACCACGAACAAGCACTTCTGTTTCCCCGGT
GAAGCTGTATAGACTGTCTCCACGGTTGAAAATGGCAGATCCGTTACCCGCTTT
GGTACTTCGAGAAACCTAGTATCACCTTGGAATCTTCGATGCGTTGCGCTCAGC
ACTCAACCCCAGAGTGTAGCTTAGGTCGATGAGTCTGGACGATCCTCACTGGT
GACAGTGGTCCAGGCTGCGTTGGCGGCCTACCTGTGGTGAAAACCACAGGACG
CTAGTTGTGAACAAGGTGTGAAGAGTCTATTGAGCTACCAAAGAGTCCTCCGG
CCCCTGAATGCGGCTAATCCTAACCACGGAGCAAGTGCCTTCAAACCAGTAGG
TAGCTTGTCGTAACGCGCAAGTCTGTGGCGGAACCGACTACTTTGGGTGTCCGT
GTTTCCTTTTATTTTATCATGGCTGCTTATGGTGACAATCTAAGATTGTTATCAT
ATAGCTATTGGATTGGCCATCCGGTGACTAACAGAGATCTCATATACCTTTTTG
TTGGTTTTACTAAATTAAATATTACCACGTTCAAGACACTTCTCCTTATCATAC
AACTAAATAGTGAGAAGAGAAA
(SEQ ID NO: 24873)
CACCCTCAGGTGCAAAGATGTTAAAACAGCTCTGGGGTTGTTCCCACCCACAG
GGCCCACTGGGCGCTAGCACTCTGGTATCCCGGTACCCTTGTGCGCCTGTTTTA
TCTACCCTTCCCCAATTGCAATTTAGAAGTTCAACACTCACGGTCAATAGACGG
CTCAGTACGCCAACTGAGTTACGACCAAGTACTTCTGTTACCCCGGACCGAGT
ATCAATAGGCTGCTCACGCGGCTGAAGGAGAAAACGTTCGTTATCCGGCCAAT
TACTTCGAGAAACCTAGTACCACCATGAAGGTTGCGCAGCGTTTCGCTCCGCA
CAACCCCAGTGTAGATCAGGCCGATGAGTCACCGCACTCCCCACGGGCGACCG
TGGCGGTGGCTGCGCTGGCGGCCTGCCCATGGGGTAACCCATGGGACGCTTCA
ATACTGACATGGTGCGAAGAGTCTATTGAGCTAATTGGTAGTCCTCCGGCCCCT
GAATGCGGCTAATCCTAACTGCGGAGCAGGTACCCACATGCCAGTGGGCAATC
TGTCGTAATGGGCAACTCTGCAGCGGAACCGACTACTTTGGGTGTCCGTGTTTC
CTTTTATTCCTATACTGGCTGCTTATGGTGACAATTGAGAGATTGTTACCATAT
AGCTATTGGATTGGCCATCCGGTGACAAACAGAGCAATTGTTTACTTATTTGTT
GGTTTTGTACCATTAAATTTTAAGGCTCTAAGAACACTTAATTACATATTATTA
CTCAACTCATTCAA
(SEQ ID NO: 24874)
TNNNNNNNNNNNNNNNNNCNNNNAAACAGCTCTGGGGTTGTTCCCACCCCAG
AGGCCCACGTGGCGGCTAGTACACTGGTATCACGGTACCTTTGTACGCCTGTTT
TATACTCCCTCCCCCGCAACTTAGAAGCATACAANTCAAGCTCAATAGGAGGG
GGTGCAAGCCAGCGCCTCCGTGGGCAAGCACTACTGTTTCCCCGGTGAGGCCG
CATAGACTGTTCCCACGGTTGAAAGTGNCCGATCCGTTATCCGCTCATGTACTT
CGAGAAGCCTAGTATCGCTCTGGAATCTTCGACGCGTTGCGCTCAGCACTCAA
CCCCGGAGTGTAGCTTGGGCCGATGAGTCTGGACAGTCCCCACTGGCGACAGT
GGTCCAGGCTGCGCTGGCGGCCCACCNGTGGCCCAAAGCCACGGGACGCTAGT
TGTGAACAGGGTGTGAAGAGCCTATTGAGCTACATGAGAGTCCTCCGGCCCCT
GAATGCGGCTAATCCTAACCATGGAGCAGGCAGCTGCAACCCAGCAGCCAGCC
TGTCGTAACGCGCAAGTCCGTGGCGGAACCGACTACTTTGGGTGTCCGTGTTTC
CTTTTATTCTTGAATGGCTGCTTATGGTGACAATCATAGATTGTTATCATAAAG
CGAGTTGGATTGGCCATCCAGTGTGAATCAGATTAATTACTCCCTTGTTTGTTG
GATCCACTCCCGAAACGTTTTACTCCTTAACTTATTGAAATTGTTTGAAGACAG
GATTTCAGTGTCACA
(SEQ ID NO: 24875)
CAANNNNNNNNNNNNNNNNNNNNTTAAAACAGCTCTGGGGTTGTTCCCACCC
CAGAGGCCCACGTGGCGGCTAGTACTCTGGTATCGCGGTACCTTTGTACGCCT
GTTTTATACCCCNACCCCATGTAACTTTAGAAGCAGTTCAACAAGTTCAATAGG
GGGGGTGCAAACCAGCACCACCACGAACAAGCACTTCTGTAACCCCGGTNACC
TTGTATANGCTGTACCCACGGCTGAAAGCGAGTGATCCGTTATCCGCTTAAGTA
CTTCGAGAAGCCTAGTANCACCTTAGAATCTTCGATGCGTTGCGCTCAGCACTC
TATCCCGAGTGTAGCTTAGGCTGATGAGTCTGGGCGTTCCNCACCGGCGACGG
TGGCCCAGGCTGCGTTGGCGGCCTACCCATGGCTAACGCCATGGGACGCTAGA
TGTGAACAAGGTGTGAAGAGCCTATTGAGCTACTTAAGAGTCCTCCGGCCCCT
GAATGCGGCTAATCCCAACCACGGAGCAGGTGCCTTCAACCCAGAGGGTGGCC
TGTCGTAACGCGCAAGTCTGTGGCGGAACCGACTACTTTGGGTGTCCGTGTTTC
CTTTTATCTTTTATATGGCTGCTTATGGTGACAATCATAGATTGTTATCATAAAG
CGACTTGGATTGGCCATCCGGTAAAATACAAACACATCANTTACTTGTTTGTTG
GATTTACTCCACTCNCCCAATTTACTCCTAGCATAATATCCATTGTTTTGTTAAN
AAGACATTACTATCACA
(SEQ ID NO: 24876)
CAACCCACCAACGGTACACCGCNCNNNTTNNNGNNNNNNNNNNNNNNNATTC
CCAAGGCCCACTCGGTTCAACTTCGGTTTCCGGACAAATANAAAGAACCTCAG
TCCTCTTGGTACTTTCTCGCCTGAGTCTACAAAGCGAGAAACCTGCCCCTCTAA
CGCCAGACGAGCGGCATAAAACTCGAACTTCTGGCACGTTCCACCACCCCTTC
TCCTATCCCAACCCCCATTGCGCTCTCAAGGTCGCGCTTTTCCGAGACTAGCTC
GGATTCAAAAAGTTCCTGGCACCCTTTACCCCTTCAGGCCCTTAAGGTAGGAA
CTGACCTTGTGCTGTGATCTCGGTGCGGAAGTGCTACTGCGTAGTGATTGTAAG
ATCCTTTTGTGGTTCTGCCCTGGCAAGGCTACAGAGTGCTGTGATCCGCTGCGG
ATGCCATCCTGGTAACAGGACCCCCAGTGTGCGCAACAGTATGTTCACGGTCT
TCCGTGTTCACCACATTCGGAACACTGCTTTCGTGAAACAGTGTGTGTCCAATC
CCTGTGATCAGTATCAACCACACCACCTAGGAATGCTAGGAAGGTACCCCGGT
TCGCCGGGATCTGATCCTAGGCTAATTGTCTACGGTGGTGCTCCTTTTTATTTTC
CACTTCAACTCACTGGTTACAACTGCTTGATTTCTGTGTTTGCTGCTTTTCTCTG
CTCTCACTGCCATTCTCAAGTGTTCACACTGTCCAAGCTCCTTTGGTTGTTCGCT
TCCACTTGCCACTGTCAACTCTTGTC
(SEQ ID NO: 24877)
GTTTGATGGCGTGNNNNNNNNNNNNNNNNNNNNTTAAAACAGCCTGTGGGTT
GTTCCCACCCACAGGGCCCACTGGGCGCTAGCACACTGATTCTGCGGGATCTTT
GTGCGCCTGTTTTATAACCCTTNCCCCTAAGCAGCAACTTAGAAGCTTTGNACA
ATCACGACCANTAGTGGGCGTGNCGCGCCAGTCACGTCTTGGTCAAGCACTTC
TGTNTCCCCGGACTGAGTATCAATAGACTGCTCACGCGGTTGAAGGAGAAAAC
GTTCGTTANCCGGCTAACTACTTCGAGAAACCTAGTAGCACCNTGAAAGTTGC
GGAGTGTTTCGCTCAGCACTTCCCCCGTGTAGATCAGGTCGATGAGTCACTGTA
AACCCCACGGGCGACCGTGACAGTGGCTGCGTTGGCGGCCTGCCCATGGGGTA
ACCCATGGGACGCTCTAATACAGACATGGTGNGAAGAGTCTATTGAGCTAGTT
AGNAGTCCTCCGGCCCCTGAATGCGGCTAATCCTAACTGCGGAGCGCGCACCC
TCAACCCAGGGGGGCGGCGNGTCGTAATGGGTAACTCTGCAGCGGAACCGACT
ACTTTGGGTGTCCGTGTTTCCTTTTTTATTCNTTATTGGCTGCTTATGGTGACAA
TTGAAAAGTTGTTACCATATAGCTATTGGATTGGCCATCCGGTGTCNAACAGA
GCTATTGTTTACCTATTTATTGGATACGTCCCTCTTAATCTCAAGGCCATTCAAA
CTCTTGATTATATATTGCTCCTTAANTGTAAGAA
(SEQ ID NO: 24878)
CAANNNNNNNNNNNNNNNNNNNNTTAAAACAGCTCTGGGGTTGTTCCCACCC
CAGAGGCCCACGTGGCGGCTAGTACTCTGGTATCGCGGTACCTTTGTACGCCT
GTTTTATACCCCNACCCCATGTAACTTTAGAAGCAGTTCAACAAGTTCAATAGG
GGGGGTGCAAACCAGCACCACCACGAACAAGCACTTCTGTAACCCCGGTGACC
TTGTATAAGCTGTACCCACGGCTGAAAGCGAGTGATCCGTTATCCGCTTAAGTA
CTTCGAGAAGCCTAGTATCACCTTAGAATCTTCGATGCGTTGCGCTCAGCACTC
TATCCCGAGTGTAGCTTAGGCTGATGAGTCTGGGCGTTCCNCACCGGCGACGG
TGGCCCAGGCTGCGTTGGCGGCCTACCCATGGCTAACGCCATGGGACGCTAGA
TGTGAACAAGGTGTGAAGAGCCTATTGAGCTACTTAAGAGTCCTCCGGCCCCT
GAATGCGGCTAATCCCAACCACGGAGCAGGTGCCTTCAACCCAGAGGGTGGCC
TGTCGTAACGCGCAAGTCTGTGGCGGAACCGACTACTTTGGGTGTCCGTGTTTC
CTTTTATCTTTTATATGGCTGCTTATGGTGACAATCATAGATTGTTATCATAAAG
CGACTTGGATTGGCCATCCGGTAAAATACAAACACATCATTTACTTGTTTGTTG
GATTTACTCCACTCTCCCAATTTACTCCTAGCATAATATCCATTGTTTTGTTAAC
AAGACATTACTATCACA
(SEQ ID NO: 24879)
ATCGAAAGGCTTATACGATCCCACGTGTGTATGCCAACCTACATGGACTCNNN
NNNNTAGGNTNNANNNNCAACCCTTTCATAACCCCCCCCTTTTAACCCNNCNN
NNNGTNANCNNACGCTTCACTCGCCTTTGGGTATAGCGGCCCAATGTGCTGAA
GAAAAGGATACGCTATAAGGGGCCAACGGGTGGTGGCCCTTAAGACCACCCA
ACCTAGAAGCTTGTACACTCGGGCAATAGTGAGGCCCACATCCAGTGGGTCAA
GCCCAAAGCATTCTTGTTCCCCGGTATGATCTCATAAGCTGTACCCACGGCTGA
AAGAGTGATTATCGTTATCCCACTCAGTACTTCGGAGAGCCTAGTACACCACTT
GGAAATGGAAGTCTGTGATCCGGGGTTGACCCTGAACCCCAGAAACTCATGAT
GAGGCTAACCTTCCCGAACACGGCGACGTGTGGTTAGCCTGCGCTGGCATGAG
GCCTCTTTGTAGGCAGACTGAAATGGAAGGGTGACGAAGAGCCGACTGAGCTA
CTGTTTTATTCCTCCGGCCCCCTGAATGCGGCTAATCCTAACTCCTGGTCCAGT
ACTTGTAACCCAACAGGTGGCTGGTCGTAATGCGTAAGCCGGGAGCGGAACCG
ACTACTTTGGGGCGTCCGTGTTTCTCAATATTATTCATTTCTAGCTTATGGTGAC
AATTTATGATTGCAGAGATTGTGCTGTATTTGTGTCTGAGAGAAGAAGTAACA
AT
(SEQ ID NO: 24880)
CAACCCACCAACGGTACACCGCNCNNNTTNNNGNNNNNNNNNNNNNNNATTC
CCAAGGCCCACTCGGTTCAACTTCGGTTTCCGGACAAATANAAAGAACCTCAG
TCCTCTTGGTACTTTCTCGCCTGAGTCTACAAAGCGAGAAACCTGCCCCTCTAA
CGCCAGACGAGCGGCATAAAACTCGAACTTCTGGCACGTTCCACCACCCCTTC
TCCTATCCCAACCCCCATTGCGCTCTCAAGGTCGCGCTTTTCCGAGACTAGCTC
GGATTCAAAAAGTTCCTGGCACCCTTTACCCCTTCAGGCCCTTAAGGTAGGAA
CTGACCTTGTGCTGTGATCTCGGTGCGGAAGTGCTACTGCGTAGTGATTGTAAG
ATCCTTTTGTGGTTCTGCCCTGGCAAGGCTACAGAGTGCTGTGATCCGCTGCGG
ATGCCATCCTGGTAACAGGACCCCCAGTGTGCGCAACAGTATGTTCACGGTCT
TCCGTGTTCACCACATTCGGAACACTGCTTTCGTGAAACAGTGTGTGTCCAATC
CCTGTGATCAGTATCAACCACACCACCTAGGAATGCTAGGAAGGTACCCCGGT
TCGCCGGGATCTGATCCTAGGCTAATTGTCTACGGTGGTGCTCCTTTTTATTTTC
CACTTCAACTCACTGGTTACAACTGCTTGATTTCTGTGTTTGCTGCTTTTCTCTG
CTCTCACTGCCATTCTCAAGTGTTCACACTGTCCAAGCTCCTTTGGTTGTTCGCT
TCCACTTGCCACTGTCAACTCTTGTC
(SEQ ID NO: 24881)
NNNNNNNNNNNNNNNNNNNNTTAAAACAGCTCTGGGGTTGNACCCACCCCAG
AGGCCCACGTGGCGGCTAGTACTCCGGTATTGCGGTACCCTTGTACGCCTGTTT
TATACTCCCTTCCCGTAACTTAGACGCACAAAACCAAGTTCAATAGAAGGGGG
TACAAACCAGTACCACCACGAACAAGCACTTCTGTTTCCCCGGTGATGTTGTAT
AGACTGCTTGCGTGGTTGAAAGCGACGGATCCGTTATCCGCTTATGTACTTCGA
GAAGCCCAGTACCACCTCGGAATCTTCGATGCGTTGCGCTCAGCACTCAACCC
CAGAGTGTAGCTTAGGCTGATGAGTCTGGACATCCCTCACCGGTGACGGTGGT
CCAGGCTGCGTTGGCGGCCTACCTATGGCTAACGCCATGGGACGCTAGTTGTG
AACAAGGTGTGAAGAGCCTATTGAGCTACATAAGAATCCTCCGGCCCCTGAAT
GCGGCTAATCCCAACCTCGGAGCAGGTGGTCACAAACCAGTGATTGGCCTGTC
GTAACGCGCAAGTCCGTGGCGGAACCGACTACTTTGGGTGTCCGTGTTTCCTTT
TATTTTATTGTGGCTGCTTATGGTGACAATCACAGATTGTTATCATAAAGCGAA
TTGGATTGGCCATCCGGTGAAAGTGAGATTCATTATCTATCTGTTTGCTGGATT
CGCTCCATTGAGTGTGTTTACTCTAAGTACAATTTCAACAGTTATTTCAATCAG
ACAATTGTATCANA
(SEQ ID NO: 24882)
ACCACCGTGAATGCGACAATGGCGCGCCTCGGCAAGGCCGCCGATACCGCACT
GTGCTACTTCGGTACCACTCTGCGGGAATGACAATATGCCCGGTGTATAGTTTG
CCTGTTTCTCGCACNNNNNNNGCNNNNNCGGNANTGTGAAACTGGCACCCCTC
CTCTCCCCTACCACCCTTTCTCCTTCGCCCCCATTCATAATTTACAACGCCGCAC
ACAGCGGCGGCCGCCAAGGGCTAGCCTGGCGGTTATAAAAGGAACCTGGGTCT
TTCCCTCTTCAAGCCAAAAGGTAGGTTCCCTGTGTCCCTGAATGCTCGGTGAGG
AATGCTGCACCGTAACGCTTTGTGAAGTGTTTGCAAGTTCTGGCCCGGCAAGCC
TACAGAGTGCTGTGATCCGCTGCGGACGCCATCCTGGTAACAGGACCCCCAGT
GTGCGCAACAGTATGTTCAGACTTCGGTTTGTTCACTTGCTTTCATGGACCATT
GCGCGAAAGTGCGTGCGCCATATCCCTGTACTTCAGGTGTGCTTCTCTGGACCC
TAGGAATGCTGCGAAGGTACCCCGTTTCGGCGGGATCTGATCGCAGGCTAATT
GTCTATGGGTTCAGTTTCCTTTTTCTTTACTCCACAATTGACTGCTTAACTGACT
CTGGATCTTGTGCTTCCACTGCTCTTTCTGCTCTCAAAACGGCTTCACTTACCAA
CTCTCACCTTTCGACCAACACCATTTACACACTAACTTTTTTCGACTCTTCTGAC
TCCTGGCTTGGTGAAGAC
(SEQ ID NO: 24883)
NNNNNNNNNNNNNNNNNNNNATTCTCGGGCTACGGCCCTGGAGCCACTCCGG
CTCCTAAAGATTTAGAAGTTTGAGCACACCCGCCCACTAGGGCCCCCCATCCA
GGGGGGCAACGGGCAAGCACTTCTGTTTCCCCGGTATGATCTGATAGGCTGTA
ACCACGGCTGAAACAGAGATTATCGTTATCCGCTTCACTACTTCGAGAAGCCT
AGTAATGATGGGTGAAATTGAATCCGTTGATCCGGTGTCTCCCCCACACCAGA
AACTCATGATGAGGGTTGCCATCCCGGCTACGGCGACGTAGCGGGCATCCCTG
CGCTGGCATGAGGCCTCTTAGGAGGACGGATGATATGGATCTTGTCGTGAAGA
GCCTATTGAGCTAGTGTCGACTCCTCCGCCCCCGTGAATGCGGCTAATCCTAAC
CCCGGAGCAGGTGGGTCCAATCCAGGGCCTGGCCTGTCGTAATGCGTAAGTCT
GGGACGGAACCGACTACTTTCGGGAAGGCGTGTTTCCATTTGTTCATTATTTGT
GTGTTTATGGTGACAACTCTGGGTAAACGTTCTATTGCGTTTATTGAGAGATTC
CCAACAATTGAACAAACGAGAACTACCTGTTTTATTAAATTTACACAGAGAAG
AATTACA
(SEQ ID NO: 24884)
ACGTTCGCTGCGGTGCCAATTTAAAACAGCTCTGGGGTTGTTCCCACCCCAGA
GGCCCACGTGGCGGCTAGTACTCTGGTATTACGGTACCTTTGTACGCCTGTTTT
ATACTCCCTTCCCCTCCGTAACTTAGAAGCATGAGAATCAAGTTCAATAGAAG
GGGGTACAAACCAGTACCACCACGAACAAGCACTTCTGTCACCCCGGTGAGGT
TGTATAGACTGTCCCCACGGTTGAAAGCGACTGATCCGTTATCCGCTCAATTAC
TTCGGGAAGCCTAGTACCATCCTGGAATCTTCGATGCGTTGCGCTCAGCACTCA
ACCCCAGAGTGTAGCTTAGGCCGATGAGTCTGGACAATCCTCACTGGCGACAG
TGGTCCAGGCTGCGTTGGCGGCCTACCTGTGGTCCAAAGCCACAGGACGCTAG
TTGTGAACAAGGTGCGAAGAGCCTACTGAGCTAATTGAGAGTCCTCCGGCCCC
TGAATGCGGCTAATCCCAACCACGGAGCAGGCGGTTGCAAACCAGCAACTGGC
TTGTCGTAACGCGAAAGTCTGTGGCGGAACCGACTACTTTGGGTGTCCGTGTTT
CCTTTTATTCTTATGATGGCTGCTTATGGTGACAATCGTTGATTGTTATCATAAA
GCGAATTGGATTGGCCATCCGGTGAAAATCAAATCTATTGTTTACCTGTTCATT
GGGTTCACTCCACTAACTTCACACACTATATATCTCATTAAGGTCGTATTACTA
GTAAGGAATTATCACCAAA
(SEQ ID NO: 24885)
CGAGCTCCGTGAATAAACTCCTTCACTTTCCATAACTACAGTGCTTTGGTAGGT
AAGCATCCTGACCCCCCGCGGAAGCTGCCAACGTGGCAACTGTGGGGATCCAG
GCAGGTTATCAAAGGCACCCGGTCTTTCCGCCTTCAGGAGTATCCCTGCCGGTG
AATTCCGACAGGGCTCTGCTTGGTGCCAACCTCCCCCAAATGCGCGCTGCGGG
AGTGCTCTTCCCCAACTCATCTTAGTAACCTCTCATGTGTGTGCTTGGTCAGCA
TATCTGAGGCGACGTTCCGCTGTCCCAGACCAGTCCAGCAATGGACGGGCCAG
TGTGCGTAGTCGCTTTCCGGTTTCCCGGCGCATGTTTGGCGAAACGCTGAGGTA
AGGTTGGTGTGCCCAATGCCCGTAATTTGGTGACACCTCAAGACCACCCAGGA
ATGCCAGGGAGGTACCCCACTTCGGTGGGATCTGACCCTGGGCTAATTGTCTA
CGGTGGTTCTTCTTGCTTCCACTTCTCTTTTTTCTGGCATG
(SEQ ID NO: 24886)
AGGAGTGCTGATATAGATTCGGATTTAGGCCGTGGTGCCCGTGGGCACGACGG
CCACGGCTCCAGCCGCACTCCTCTCCACGCCTTGTCCCACGGGACATAAAGTG
GAGCCCTAAGCCCTAAACCCCCATGTTACCCAATTTTCTTATCCACTTCCCTTTT
TCCCTTTGACTCACATTTTTCTTGACCGCGCTTGTGAGGCGGTTCCCGTACAGTT
GAGTTGACGGGTCCCGCGCGGGACCTGAAAATTACTCCATACTGATTAGGAAA
GGTTCTAGTACGCTTGTACCCTTAAGGCCAGCGGGCTACCTCCTTTAGGTCTTA
TAGCCCCAACTGGTTAGAACCACTCCAGCTCACCTCTATCGGTGCGGGAGTGC
TACCACCCCTGCCTGCGTCAAGCCCGTTTTTCTAGTGATCTGTCCACAAACACT
TGGATTCCCGTCGCGACTGACAGAACCTGGCGACAGGTGTCTCGATGTGTGTG
ACCAAGTGGCATGAGAATCGGAAACTAACGCTGGTAAGATGGTAAGTGCCCA
ATCCCTGTGGGGTGATGTTTTTCTCTGCATAGGAATGCTGCGAAGGTACCCCGG
CCTTGAGTCGGGATCTGATCGCAGGCTAATTGTCCTCTGTGCAGTTCATTTCCT
TTTCTTTTCATTGGCATC
(SEQ ID NO: 24887)
NNNNNNNNNNNNNNNNNNNNNTNNNNNNNNNNNNNNNNNNNNNNCACTCAC
AGGACCCACGCGGTGCTAGCACTCTGGTTCCACGGNACCTTTGTGCGCCTGTTT
TANGTCCCCTCTCCCCAATTTGTAACTTAGAAGCAATTTNCAACACTGATCANT
AGCAGGCATNGCGCACCAGCTATGTCTTGATCAAGCACTTCTGTTTCCCCGGAC
CGAGTATCAATAGACTGTTCACACGGTTGAAGGAGAAAGCGTCCGTTATCCGG
CTAACTACTTCGAGAAACCCAGTAGCNCCATTGAAACTNCAGAGTGTTTCGCT
CCACACTTCCCCCGTGTAGATCAGGTCGATGAGTCACTGCAATCCCCACGGGC
GACCGTGGCAGTGGCTGCGCTGGCGGCCTGCCTATGGGGCAACCCATAGGACG
CTCTAATGTGGACATGGTGNGAAGAGTCTATTGAGCTAGTTAGTAGTCCTCCG
GCCCCTGAATGCGGCTAATCCTAACTGCGGAGCACATGCCTTCAANCCAGAGG
GTAGTGTGTCGTAATGGGCAACTCTGCAGCGGAACCGACTACTTTGGGTGTCC
GTGTTTCCTTTTATTCTCACATTGGCTGCTTATGGTGACAATTACAGAATTGTTA
CCATATAGCTATTGGATTGGCCATCCGGTGTGCAATAGAGCTATTATATACCTA
TTTGTTGGCTTTGTACCACTNACCTTAAAATCTNTAACCACTCTNGAGTNTATA
TTAACCCTTAATACAATCAAAC
(SEQ ID NO: 24888)
NNNNNNNNNNNNNNNNNNNNNTAAAANNGCCTTGGGGTTGTTCCCACTCCAA
GGGCCCACGTGGCGGCTAGTACTCTGGTACTTCGGTACCTTTGTACGCCTGTTT
TATCTCCCTTCCCAATGTAANTTAGAAGNTCTTAAATCAANGCTCAATAGGTGG
GGCGCAAACCAGCGCTCTCATGAGCAAGCACTCCTGTCTCCCCGGTGNGGTTG
TATAAACTGTTCCCACGGTTGAAAACAACCTATCCGTTATCCGCTATAGTACTT
CGAGAAACCTAGTATCACCTTTGGATTGTTGACGCGTTGCGCTCAGCACACTAA
CCCGTGTGTAGCTTGGGTCGATGAGTCTGGACATACCCCACTGGCGACAGTGG
TCCAGGCTGCGTTGGCGGCCTACTCATGGTGAAAACCATGAGACGCTAGACAT
GAACAAGGTGTGAAGAGTCTATTGAGCTACTATAGAGTCCTCCGGCCCCTGAA
TGCGGCTAATCCTAACCATGGAGCAAGTGCTCACAGGCCAGTGAGTTGCTTGT
CGTAATGCGCAAGTCCGTGGCGGAACCGACTACTTTGGGTGTCCGTGTTTCACT
TTTTACTTTTATGACTGCTTATGGTGACAATTTGATATTGTTACCATTTAGCTTG
TCAAATNAATTGCAAAAGATCCTAAATCTTANTTANCAANTTGCATTTTGATAA
CNTTNATTTGAAAATTTTAANANNNNNNTNAANNA
(SEQ ID NO: 24889)
AGATACTATAGATGGATCCACCTCGGCCCCCTCACCCTCTTTTCCGGTGGCCAC
GCCCGGGCCACCGATACTTCCCTTNNCNNNNNCGGGANTNNTNNNNNGNAAC
NCNACANNNNNNNNNTNTTTNNNNNNNCNNTCCCCCTTTTCCNAACCCCAACC
GCCGTATCTGGTGGCGGCAAGACACACGGGTCTTTCCCTCTAAAGCACAATTG
TGTGTGTGTCCCAGGTCCTCCTGCGTNCGGTGCGGGAGTGCTCCCACCCAACTG
TTGTAAGCCTGTCCAACGNGTCGTCCTGGCAAGACTATGACGTCGCATGTTCCG
CTGCGGATGCCGACCGGGTAACCGGTTCCCCAGTGTGTGTAGTGCGATCTTCC
AGGTNCTCCTGGTTGGCGTTGTCCAGAAACTGCTTCAGGTAAGTGGGGTGTGC
CCAATCCCTACAAAGGTTGATTCTTTCACCACCTTAGGAATGCTCCGGAGGTAC
CCCAGCAACAGCTGGGATCTGACCGGAGGCTAATTGTCTACGGGTGGTGTTTC
CTTTTTCTTTTTCACACAACTNNANTGCTGACAACTCACTGACTANCCACTTGC
TCTCTTGTGCCTTTCTGCTCTGGTTCAAGTTCCTTGATTGTTTTTGANTGCTTTTT
NANTGCTTTTCTTCNCACAATCCTTGCTCAGTTCAAAGTC
(SEQ ID NO: 24890)
TTGAGTGGCCTCACCGGAATTGTGAAACTNGNNNNNNNNNNNNNCCCTNCCAC
CCTTTCTCCTTCGCCCCCATTCATAANTNNNNNCGNNNNNNNNNNCGGCGGCC
GCCAAGGGCTAGCCTGGCGGTTATAAAAGGAACCTGGGTCTTTCCCTCTTCAA
GCCAAAAGGTAGGTTCCCTGTGTCCCTGAATGCTCGGTGAGGAATGCTGCACC
GTAACGCTTTGTGAAGTGTTTGCAAGTTCTGGCCCGGCAAGCCTACAGAGTGCT
GTGATCCGCTGCGGACGCCATCCTGGTAACAGGACCCCCAGTGTGCGCAACAG
TATGTTCAGACTTCGGTTTGTTCACTTGCTTTCATGGACCATTGCGCGAAAGTG
CGTGCGCCATATCCCTGTACTTCAGGTGTGCTTCTCTGGACCCTAGGAATGCTG
CGAAGGTACCCCGTTTCGGCGGGATCTGATCGCAGGCTAATTGTCTATGGGTTC
AGTTTCCTTTTTCTTTACTCCACAATTGACTGCTTAACTGACTCTGGATCTTGTG
CTTCCACTGCTCTTTCTGCTCTCAAAACGGCTTCACTTACCAACTCTCACCTTTC
GACCAACACCATTTACACACTAACTTTTTTCGACTCTTCTGACTCCTGGCTTGGT
GAAGAC
(SEQ ID NO: 24891)
NNNNNNNNNNNNNNNNNANNTTNAAANNNNNNNNGGGGNTGTTCCCACCCTA
GAGGCCCACGTGGCGGCTAGCACTCTGGTATCACGGTACCTTTGTGCGCCTGTT
TTATATCCCTTTCCCCATGCAACTTAGAAGTTTTTTAAACAAAGTTCAATAGAA
GGGGGTACAAACCAGTGCCACCACGAACAAGCACTTCTGTTTCCCCGGTGAAG
CTGCATAGACTGTCCCCACGGTTGAAAGCGGCAGATCCGTNATCCGCTTTGGT
ACTTCGAGAAACCTAGTATCACCTTGGAATCTTCGATGCGTTGCGCTCAGCACT
CAACCCCAGAGTGTAGCTTAGGTTGATGAGTCTGGACNATCCTCACTGGCGAC
AGTGGTCCAGGCTGCGTTGGCGGCCTACCTGTGGTGAAAACCACAGGACGCTA
GATGTGAACAAGGTGTGAAGAGTCTATTGAGCTACCAAAGAGTCCTCCGGCCC
CTGAATGCGGCTAATCCTAACCACGGAGCAAGTACCCACAAACCAGTGGGCAG
CTTGTCGTAACGCGCAAGTCTGTGGCGGAACCGACTACTTTGGGTGTCCGTGTT
TCCTTTTTATTTTATCATGGCTGCTTATGGTGACAATCTAAGATTGTTATCATAT
AGCTATTGGATTGGCCATCCGGTGACTAACAGAGATATTATATACCTATTTGTT
GGTTTTNTTAGATTAAATACTATCACATTCAAAACTCTTCTTTATATCATACAAT
TGAACAGTAGAAAGAGAAA
(SEQ ID NO: 24892)

c. Termination Sequence

In certain embodiments, the core functional element comprises a termination sequence. In some embodiments, the termination sequence comprises a stop codon. In one embodiment, the termination sequence comprises a stop cassette. In some embodiments, the stop cassette comprises at least 2 stop codons. In some embodiments, the stop cassette comprises at least 2 frames of stop codons. In the same embodiment, the frames of the stop codons in a stop cassette each comprise 1, 2 or more stop codons. In some embodiments, the stop cassette comprises a LoxP or a RoxStopRox, or frt-flanked stop cassette. In the same embodiment, the stop cassette comprises a lox-stop-lox stop cassette.

### C. Variants

In certain embodiments, a provided polynucleotide (e.g., a DNA template, a precursor RNA polynucleotide, or a circular RNA polynucleotide) comprises modified nucleotides and/or modified nucleosides. In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine). In other embodiments, the modified nucleoside is m¹A (1-methyladenosine); m²A (2-methyladenosine); Am (2′-O-methyladenosine); ms² m⁶A (2-methylthio-N⁶-methyladenosine); i⁶A (N⁶-isopentenyladenosine); ms²i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t6A (2-methylthio-N⁶-threonyl carbamoyladenosine); m t A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); miIm (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine(phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); nCm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ 2A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ 2Am (N⁶,N⁶,0-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); fVCm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In some embodiments, the modified nucleoside may include a compound selected from the group of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In another embodiment, the modifications are independently selected from the group consisting of 5-methylcytosine, pseudouridine and 1-methylpseudouridine.

In some embodiments, the modified ribonucleosides include 5-methylcytidine, 5-methoxyuridine, 1-methyl-pseudouridine, N6-methyladenosine, and/or pseudouridine. In some embodiments, such modified nucleosides provide additional stability and resistance to immune activation.

In particular embodiments, polynucleotides may be codon-optimized. A codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the core functional element.

### 3. Payloads

In some embodiments, the expression sequence encodes a therapeutic protein. In some embodiments, the therapeutic protein is selected from the proteins listed in Table 1.

TABLE 1
Payloads

| | | Tar- | |
|---|---|---|---|
| | | get | |
| | | cell/ | |
| Pay- | | or- | |
| load | Sequence | gan | Preferred delivery formulation |
| CD19 CAR | Any of sequences selected from SEQ ID NOs: 3153- 3161 | T cells | 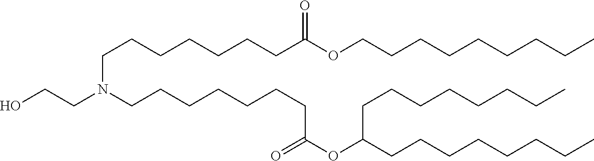 [SMILES: CCCCCCCCCOC(=O)CCCCCCCN(CCO)CCCCCCCC(=O)OC(CCCCCCCC)CCCCCCCC] |
| | | | (50 mol %) |
| | | | DSPC (10 mol %) |
| | | | Beta-sitosterol (28.5% mol %) |
| | | | Cholesterol (10 mol %) |
| | | | PEG DMG (1.5 mol %) |
| BCMA CAR* | MALPVTALLLPLALLL HAARPDIVLTQSPASLA VSLGERATINCRASESV SVIGAHLIHWYQQKPG QPPKLLIYLASNLETGV PARFSGSGSGTDFTLTIS SLQAEDAAIYYCLQSRI FPRTFGQGTKLEIKGST SGSGKPGSGEGSTKGQ VQLVQSGSELKKPGAS | T cells | 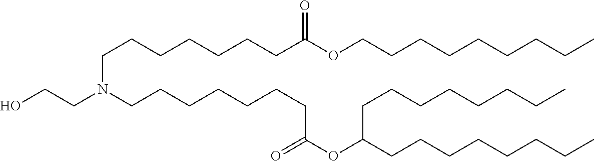 [SMILES: CCCCCCCCCOC(=O)CCCCCCCN(CCO)CCCCCCCC(=O)OC(CCCCCCCC)CCCCCCCC] |
| | VKVSCKASGYTFTDYSI | | |
| | NWVRQAPGQGLEWMG | | (50 mol %) |
| | WINTETREPAYAYDFR | | DSPC (10 mol %) |
| | GRFVFSLDTSVSTAYLQ | | Beta-sitosterol (28.5% mol %) |
| | ISSLKAEDTAVYYCAR | | Cholesterol (10 mol %) |
| | DYSYAMDYWGQGTLV | | PEG DMG (1.5 mol %) |
| | TVSSAAATTTPAPRPPT | | |
| | PAPTIASQPLSLRPEACR | | |
| | PAAGGAVHTRGLDFAC | | |
| | DIYIWAPLAGTCGVLLL | | |
| | SLVITLYCKRGRKKLLY | | |
| | IFKQPFMRPVQTTQEED | | |
| | GCSCRFPEEEEGGCELR | | |
| | VKFSRSADAPAYQQGQ | | |
| | NQLYNELNLGRREEYD | | |
| | VLDKRRGRDPEMGGKP | | |
| | RRKNPQEGLYNELQKD | | |
| | KMAEAYSEIGMKGERR | | |
| | RGKGHDGLYQGLSTAT | | |
| | KDTYDALHMQALPPR | | |
| | (SEQ ID NO: 3270) | | |
| | *The BCMA CAR may be | | |
| | chosen from any of the | | |
| | anti-BCMA CARs | | |
| | disclosed in US Patent | | |
| | Application US | | |
| | 2021/0128618A1 | | |
| MAGE- A4 TCR | TCR alpha chain: KNQVEQSPQSLIILEGK NCTLQCNYTVSPFSNLR WYKQDTGRGPVSLTIM TFSENTKSNGRYTATLD ADTKQSSLHITASQLSD SASYICVVNHSGGSYIP TFGRGTSLIVHPYIQKP DPAVYQLRDSKSSDKS VCLFTDFDSQTNVSQSK | T cells | 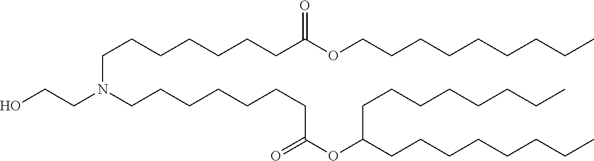 [SMILES: CCCCCCCCCOC(=O)CCCCCCCN(CCO)CCCCCCCC(=O)OC(CCCCCCCC)CCCCCCCC] |
| | DSDVYITDKTVLDMRS | | |
| | MDFKSNSAVAWSNKS | | (50 mol %) |
| | DFACANAFNNSIIPEDT | | DSPC (10 mol %) |
| | FFPSPESS (SEQ ID NO: | | Beta-sitosterol (28.5% mol %) |
| | 3271) | | Cholesterol (10 mol %) |
| | TCR beta chain: | | PEG DMG (1.5 mol %) |
| | DVKVTQSSRYLVKRTG | | |
| | EKVFLECVQDMDHEN | | |
| | MFWYRQDPGLGLRLIY | | |
| | FSYDVKMKEKGDIPEG | | |
| | YSVSREKKERFSLILES | | |
| | ASTNQTSMYLCASSFL | | |
| | MTSGDPYEQYFGPGTR | | |
| | LTVTEDLKNVFPPEVA | | |
| | VFEPSEAEISHTQKATL | | |
| | VCLATGFYPDHVELSW | | |
| | WVNGKEVHSGVSTDPQ | | |
| | PLKEQPALNDSRYCLSS | | |
| | RLRVSATFWQNPRNHF | | |
| | RCQVQFYGLSENDEWT | | |
| | QDRAKPVTQIVSAEAW | | |
| | GRAD (SEQ ID NO: | | |
| | 3272) | | |
| NY- ESO TCR | TCRalpha extracellular sequence MQEVTQIPAALSVPEGE NLVLNCSFTDSAIYNLQ WFRQDPGKGLTSLLLIQ SSQREQTSGRLNASLDK SSGRSTLYIAASQPGDS ATYLCAVRPTSGGSYIP TFGRGTSLIVHPY (SEQ ID NO: 3273) | T cells | 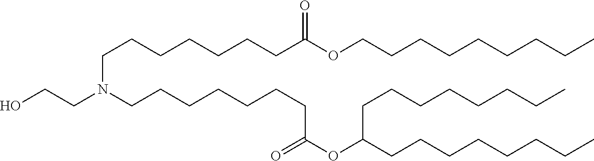 [SMILES: CCCCCCCCCOC(=O)CCCCCCCN(CCO)CCCCCCCC(=O)OC(CCCCCCCC)CCCCCCCC] |
| | TCRbeta extracellular | | |
| | sequence | | (50 mol %) |
| | MGVTQTPKFQVLKTGQ | | DSPC (10 mol %) |
| | SMTLQCAQDMNHEYM | | Beta-sitosterol (28.5% mol %) |
| | SWYRQDPGMGLRLIHY | | Cholesterol (10 mol %) |
| | SVGAGITDQGEVPNGY | | PEG DMG (1.5 mol %) |
| | NVSRSTTEDFPLRLLSA | | |
| | APSQTSVYFCASSYVG | | |
| | NTGELFFGEGSRLTVL | | |
| | (SEQ ID NO: 3274) | | |
| EPO | APPRLICDSRVLERYLL | Kid- | |
| | EAKEAENITTGCAEHCS | ney | |
| | LNENITVPDTKVNFYA | or | |
| | WKRMEVGQQAVEVW | bone | |
| | QGLALLSEAVLRGQAL | mar- | |
| | LVNSSQPWEPLQLHVD | row | |
| | KAVSGLRSLTTLLRALG | | |
| | AQKEAISPPDAASAAPL | | |
| | RTITADTFRKLFRVYSN | | |
| | FLRGKLKLYTGEACRT | | |
| | GDR (SEQ ID NO: 3275) | | |
| PAH | MSTAVLENPGLGRKLS DFGQETSYIEDNCNQN GAISLIFSLKEEVGALA KVLRLFEENDVNLTHIE SRPSRLKKDEYEFFTHL DKRSLPALTNIIKILRHD IGATVHELSRDKKKDT VPWFPRTIQELDRFANQ ILSYGAELDADHPGFKD PVYRARRKQFADIAYN YRHGQPIPRVEYMEEE | Hep- atic cells | 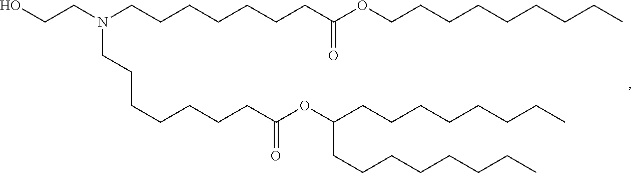 [SMILES: CCCCCCCCCOC(=O)CCCCCCCN(CCO)CCCCCCCC(=O)OC(CCCCCCCC)CCCCCCCC] |
| | KKTWGTVFKTLKSLYK | | |
| | THACYEYNHIFPLLEKY | | (50 mol %) |
| | CGFHEDNIPQLEDVSQF | | DSPC (10 mol %) |
| | LQTCTGFRLRPVAGLLS | | Cholesterol (38.5% mol %) |
| | SRDFLGGLAFRVFHCT | | PEG-DMG (1.5%) |
| | QYIRHGSKPMYTPEPDI | | OR |
| | CHELLGHVPLFSDRSFA | | MC3 (50 mol %) |
| | QFSQEIGLASLGAPDEY | | DSPC (10 mol %) |
| | IEKLATIYWFTVEFGLC | | Cholesterol (38.5% mol %) |
| | KQGDSIKAYGAGLLSSF | | PEG-DMG (1.5%) |
| | GELQYCLSEKPKLLPLE | | |
| | LEKTAIQNYTVTEFQPL | | |
| | YYVAESFNDAKEKVRN | | |
| | FAATIPRPFSVRYDPYT | | |
| | QRIEVLDNTQQLKILAD | | |
| | SINSEIGILCSALQKIK | | |
| | (SEQ ID NO: 3257) | | |
| CPS1 | LSVKAQTAHIVLEDGT KMKGYSFGHPSSVAGE VVFNTGLGGYPEAITDP AYKGQILTMANPIIGNG GAPDTTALDELGLSKY LESNGIKVSGLLVLDYS KDYNHWLATKSLGQW LQEEKVPAIYGVDTRM LTKIIRDKGTMLGKIEF EGQPVDFVDPNKQNLI AEVSTKDVKVYGKGNP | Hep- atic cells | 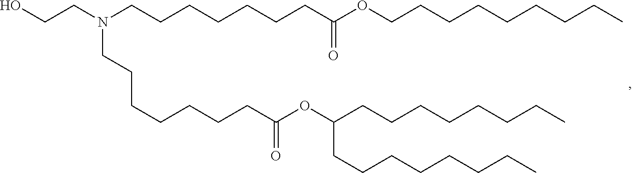 [SMILES: CCCCCCCCCOC(=O)CCCCCCCN(CCO)CCCCCCCC(=O)OC(CCCCCCCC)CCCCCCCC] |
| | TKVVAVDCGIKNNVIR | | |
| | LLVKRGAEVHLVPWN | | (50 mol %) |
| | HDFTKMEYDGILIAGGP | | DSPC (10 mol %) |
| | GNPALAEPLIQNVRKIL | | Cholesterol (38.5% mol %) |
| | ESDRKEPLFGISTGNLIT | | PEG-DMG (1.5%) |
| | GLAAGAKTYKMSMAN | | OR |
| | RGQNQPVLNITNKQAFI | | MC3 (50 mol %) |
| | TAQNHGYALDNTLPAG | | DSPC (10 mol %) |
| | WKPLFVNVNDQTNEGI | | Cholesterol (38.5% mol %) |
| | MHESKPFFAVQFHPEV | | PEG-DMG (1.5%) |
| | TPGPIDTEYLFDSFFSLI | | |
| | KKGKATTITSVLPKPAL | | |
| | VASRVEVSKVLILGSGG | | |
| | LSIGQAGEFDYSGSQAV | | |
| | KAMKEENVKTVLMNP | | |
| | NIASVQTNEVGLKQAD | | |
| | TVYFLPITPQFVTEVIKA | | |
| | EQPDGLILGMGGQTAL | | |
| | NCGVELFKRGVLKEYG | | |
| | VKVLGTSVESIMATED | | |
| | ROLFSDKLNEINEKIAPS | | |
| | FAVESIEDALKAADTIG | | |
| | YPVMIRSAYALGGLGS | | |
| | GICPNRETLMDLSTKAF | | |
| | AMTNQILVEKSVTGWK | | |
| | EIEYEVVRDADDNCVT | | |
| | VCNMENVDAMGVHTG | | |
| | DSVVVAPAQTLSNAEF | | |
| | QMLRRTSINVVRHLGIV | | |
| | GECNIQFALHPTSMEYC | | |
| | IIEVNARLSRSSALASK | | |
| | ATGYPLAFIAAKIALGIP | | |
| | LPEIKNVVSGKTSACFE | | |
| | PSLDYMVTKIPRWDLD | | |
| | RFHGTSSRIGSSMKSVG | | |
| | EVMAIGRTFEESFQKAL | | |
| | RMCHPSIEGFTPRLPMN | | |
| | KEWPSNLDLRKELSEPS | | |
| | STRIYAIAKAIDDNMSL | | |
| | DEIEKLTYIDKWFLYK | | |
| | MRDILNMEKTLKGLNS | | |
| | ESMTEETLKRAKEIGFS | | |
| | DKQISKCLGLTEAQTRE | | |
| | LRLKKNIHPWVKQIDTL | | |
| | AAEYPSVTNYLYVTYN | | |
| | GQEHDVNFDDHGMMV | | |
| | LGCGPYHIGSSVEFDW | | |
| | CAVSSIRTLRQLGKKTV | | |
| | VVNCNPETVSTDFDEC | | |
| | DKLYFEELSLERILDIYH | | |
| | QEACGGCIISVGGQIPN | | |
| | NLAVPLYKNGVKIMGT | | |
| | SPLQIDRAEDRSIFSAVL | | |
| | DELKVAQAPWKAVNT | | |
| | LNEALEFAKSVDYPCLL | | |
| | RPSYVLSGSAMNVVFS | | |
| | EDEMKKFLEEATRVSQ | | |
| | EHPVVLTKFVEGAREV | | |
| | EMDAVGKDGRVISHAI | | |
| | SEHVEDAGVHSGDATL | | |
| | MLPTQTISQGAIEKVKD | | |
| | ATRKIAKAFAISGPFNV | | |
| | QFLVKGNDVLVIECNL | | |
| | RASRSFPFVSKTLGVDF | | |
| | IDVATKVMIGENVDEK | | |
| | HLPTLDHPIIPADYVAIK | | |
| | APMFSWPRLRDADPILR | | |
| | CEMASTGEVACFGEGI | | |
| | HTAFLKAMLSTGFKIPQ | | |
| | KGILIGIQQSFRPRFLGV | | |
| | AEQLHNEGFKLFATEA | | |
| | TSDWLNANNVPATPVA | | |
| | WPSQEGQNPSLSSIRKLI | | |
| | RDGSIDLVINLPNNNTK | | |
| | FVHDNYVIRRTAVDSGI | | |
| | PLLTNFQVTKLFAEAV | | |
| | QKSRKVDSKSLFHYRQ | | |
| | YSAGKAA (SEQ ID NO: | | |
| | 3276) | | |
| Cas9 | MKRNYILGLDIGITSVG YGIIDYETRDVIDAGVR LFKEANVENNEGRRSK RGARRLKRRRRHRIQR VKKLLFDYNLLTDHSE LSGINPYEARVKGLSQK LSEEEFSAALLHLAKRR GVHNVNEVEEDTGNEL STKEQISRNSKALEEKY VAELQLERLKKDGEVR | Im- mune cells | 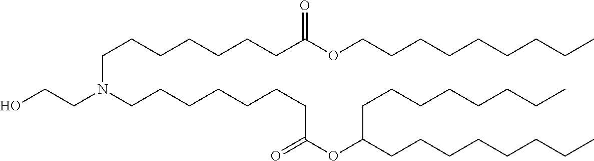 [SMILES: CCCCCCCCCOC(=O)CCCCCCCN(CCO)CCCCCCCC(=O)OC(CCCCCCCC)CCCCCCCC] |
| | GSINRFKTSDYVKEAK | | |
| | QLLKVQKAYHQLDQSF | | (50 mol %) |
| | IDTYIDLLETRRTYYEG | | DSPC (10 mol %) |
| | PGEGSPFGWKDIKEWY | | Beta-sitosterol (28.5% mol %) |
| | EMLMGHCTYFPEELRS | | Cholesterol (10 mol %) |
| | VKYAYNADLYNALND | | PEG DMG (1.5 mol %) |
| | LNNLVITRDENEKLEYY | | |
| | EKFQIIENVFKQKKKPT | | |
| | LKQIAKEILVNEEDIKG | | |
| | YRVTSTGKPEFTNLKV | | |
| | YHDIKDITARKEIIENAE | | |
| | LLDQIAKILTIYQSSEDI | | |
| | QEELTNLNSELTQEEIE | | |
| | QISNLKGYTGTHNLSLK | | |
| | AINLILDELWHTNDNQI | | |
| | AIFNRLKLVPKKVDLSQ | | |
| | QKEIPTTLVDDFILSPVV | | |
| | KRSFIQSIKVINAIIKKY | | |
| | GLPNDIIIELAREKNSKD | | |
| | AQKMINEMQKRNRQT | | |
| | NERIEEIIRTTGKENAKY | | |
| | LIEKIKLHDMQEGKCLY | | |
| | SLEAIPLEDLLNNPFNY | | |
| | EVDHIIPRSVSFDNSFNN | | |
| | KVLVKQEENSKKGNRT | | |
| | PFQYLSSSDSKISYETFK | | |
| | KHILNLAKGKGRISKTK | | |
| | KEYLLEERDINRFSVQK | | |
| | DFINRNLVDTRYATRG | | |
| | LMNLLRSYFRVNNLDV | | |
| | KVKSINGGFTSFLRRK | | |
| | WKFKKERNKGYKHHA | | |
| | EDALIIANADFIFKEWK | | |
| | KLDKAKKVMENQMFE | | |
| | EKQAESMPEIETEQEYK | | |
| | EIFITPHQIKHIKDFKDY | | |
| | KYSHRVDKKPNRELIN | | |
| | DTLYSTRKDDKGNTLI | | |
| | VNNLNGLYDKDNDKL | | |
| | KKLINKSPEKLLMYHH | | |
| | DPQTYQKLKLIMEQYG | | |
| | DEKNPLYKYYEETGNY | | |
| | LTKYSKKDNGPVIKKIK | | |
| | YYGNKLNAHLDITDDY | | |
| | PNSRNKVVKLSLKPYR | | |
| | FDVYLDNGVYKFVTVK | | |
| | NLDVIKKENYYEVNSK | | |
| | CYEEAKKLKKISNQAEF | | |
| | IASFYNNDLIKINGELY | | |
| | RVIGVNNDLLNRIEVN | | |
| | MIDITYREYLENMNDK | | |
| | RPPRIIKTIASKTQSIKK | | |
| | YSTDILGNLYEVKSKK | | |
| | HPQIIKKG (SEQ ID | | |
| | NO: 3277) | | |
| ADAM TS13 | AAGGILHLELLVAVGP DVFQAHQEDTERYVLT NLNIGAELLRDPSLGAQ FRVHLVKMVILTEPEG APNITANLTSSLLSVCG WSQTINPEDDTDPGHA DLVLYITRFDLELPDGN RQVRGVTQLGGACSPT WSCLITEDTGFDLGVTI AHEIGHSFGLEHDGAPG SGCGPSGHVMASDGAA | Hep- atic cells | 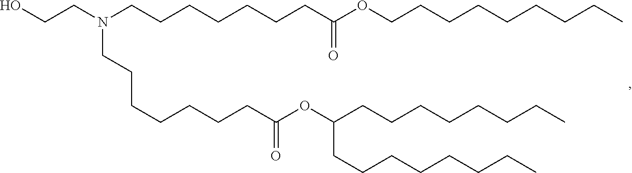 [SMILES: CCCCCCCCCOC(=O)CCCCCCCN(CCO)CCCCCCCC(=O)OC(CCCCCCCC)CCCCCCCC] |
| | PRAGLAWSPCSRRQLL | | |
| | SLLSAGRARCVWDPPR | | (50 mol %) |
| | PQPGSAGHPPDAQPGL | | DSPC (10 mol %) |
| | YYSANEQCRVAFGPKA | | Cholesterol (38.5% mol %) |
| | VACTFAREHLDMCQAL | | PEG-DMG (1.5%) |
| | SCHTDPLDQSSCSRLLV | | OR |
| | PLLDGTECGVEKWCSK | | MC3 (50 mol %) |
| | GRCRSLVELTPIAAVHG | | DSPC (10 mol %) |
| | RWSSWGPRSPCSRSCG | | Cholesterol (38.5% mol %) |
| | GGVVTRRRQCNNPRPA | | PEG-DMG (1.5%) |
| | FGGRACVGADLQAEM | | |
| | CNTQACEKTQLEFMSQ | | |
| | QCARTDGQPLRSSPGG | | |
| | ASFYHWGAAVPHSQG | | |
| | DALCRHMCRAIGESFIM | | |
| | KRGDSFLDGTRCMPSG | | |
| | PREDGTLSLCVSGSCRT | | |
| | FGCDGRMDSQQVWDR | | |
| | CQVCGGDNSTCSPRKG | | |
| | SFTAGRAREYVTFLTVT | | |
| | PNLTSVYIANHRPLFTH | | |
| | LAVRIGGRYVVAGKMS | | |
| | ISPNTTYPSLLEDGRVE | | |
| | YRVALTEDRLPRLEEIRI | | |
| | WGPLQEDADIQVYRRY | | |
| | GEEYGNLTRPDITFTYF | | |
| | QPKPRQAWVWAAVRG | | |
| | PCSVSCGAGLRWVNYS | | |
| | CLDQARKELVETVQCQ | | |
| | GSQQPPAWPEACVLEP | | |
| | CPPYWAVGDFGPCSAS | | |
| | CGGGLRERPVRCVEAQ | | |
| | GSLLKTLPPARCRAGA | | |
| | QQPAVALETCNPQPCP | | |
| | ARWEVSEPSSCTSAGG | | |
| | AGLALENETCVPGADG | | |
| | LEAPVTEGPGSVDEKLP | | |
| | APEPCVGMSCPPGWGH | | |
| | LDATSAGEKAPSPWGSI | | |
| | RTGAQAAHVWTPAAG | | |
| | SCSVSCGRGLMELRFLC | | |
| | MDSALRVPVQEELCGL | | |
| | ASKPGSRREVCQAVPC | | |
| | PARWQYKLAACSVSCG | | |
| | RGVVRRILYCARAHGE | | |
| | DDGEEILLDTQCQGLPR | | |
| | PEPQEACSLEPCPPRWK | | |
| | VMSLGPCSASCGLGTA | | |
| | RRSVACVQLDQGQDVE | | |
| | VDEAACAALVRPEASV | | |
| | PCLIADCTYRWHVGTW | | |
| | MECSVSCGDGIQRRRD | | |
| | TCLGPQAQAPVPADFC | | |
| | QHLPKPVTVRGCWAGP | | |
| | CVGQGTPSLVPHEEAA | | |
| | APGRTTATPAGASLEW | | |
| | SQARGLLFSPAPQPRRL | | |
| | LPGPQENSVQSSACGR | | |
| | QHLEPTGTIDMRGPGQ | | |
| | ADCAVAIGRPLGEVVT | | |
| | LRVLESSLNCSAGDML | | |
| | LLWGRLTWRKMCRKL | | |
| | LDMTFSSKTNTLVVRQ | | |
| | RCGRPGGGVLLRYGSQ | | |
| | LAPETFYRECDMQLFG | | |
| | PWGEIVSPSLSPATSNA | | |
| | GGCRLFINVAPHARIAI | | |
| | HALATNMGAGTEGAN | | |
| | ASYILIRDTHSLRTTAFH | | |
| | GQQVLYWESESSQAEM | | |
| | EFSEGFLKAQASLRGQ | | |
| | YWTLQSWVPEMQDPQ | | |
| | SWKGKEGT (SEQ ID | | |
| | NO: 3278) | | |
| FOXP3 | MPNPRPGKPSAPSLALG PSPGASPSWRAAPKAS DLLGARGPGGTFQGRD LRGGAHASSSSLNPMPP SQLQLPTLPLVMVAPSG ARLGPLPHLQALLQDR PHFMHQLSTVDAHART PVLQVHPLESPAMISLT PPTTATGVFSLKARPGL PPGINVASLEWVSREPA | Im- mune cells | 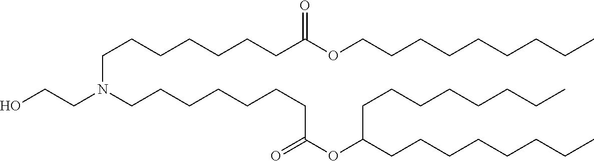 [SMILES: CCCCCCCCCOC(=O)CCCCCCCN(CCO)CCCCCCCC(=O)OC(CCCCCCCC)CCCCCCCC] |
| | LLCTFPNPSAPRKDSTL | | |
| | SAVPQSSYPLLANGVC | | (50 mol %) |
| | KWPGCEKVFEEPEDFL | | DSPC (10 mol %) |
| | KHCQADHLLDEKGRA | | Beta-sitosterol (28.5% mol %) |
| | QCLLQREMVQSLEQQL | | Cholesterol (10 mol %) |
| | VLEKEKLSAMQAHLAG | | PEG DMG (1.5 mol %) |
| | KMALTKASSVASSDKG | | |
| | SCCIVAAGSQGPVVPA | | |
| | WSGPREAPDSLFAVRR | | |
| | HLWGSHGNSTFPEFLH | | |
| | NMDYFKFHNMRPPFTY | | |
| | ATLIRWAILEAPEKQRT | | |
| | LNEIYHWFTRMFAFFR | | |
| | NHPATWKNAIRHNLSL | | |
| | HKCFVRVESEKGAVWT | | |
| | VDELEFRKKRSQRPSRC | | |
| | SNPTPGP (SEQ ID NO: | | |
| | 3187) | | |
| IL-10 | SPGQGTQSENSCTHFPG NLPNMLRDLRDAFSRV KTFFQMKDQLDNLLLK ESLLEDFKGYLGCQALS EMIQFYLEEVMPQAEN QDPDIKAHVNSLGENL KTLRLRLRRCHRFLPCE NKSKAVEQVKNAFNKL QEKGIYKAMSEFDIFIN YIEAYMTMKIRN (SEQ ID NO: 3181) | Im- mune cells | 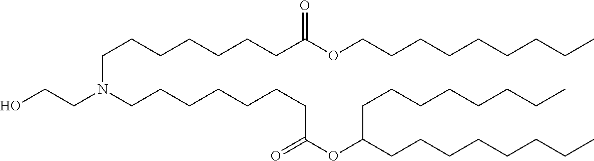 [SMILES: CCCCCCCCCOC(=O)CCCCCCCN(CCO)CCCCCCCC(=O)OC(CCCCCCCC)CCCCCCCC] |
| | | | (50 mol %) |
| | | | DSPC (10 mol %) |
| | | | Beta-sitosterol (28.5% mol %) |
| | | | Cholesterol (10 mol %) |
| | | | PEG DMG (1.5 mol %) |
| IL-2 | APTSSSTKKTQLQLEHL LLDLQMILNGINNYKNP KLTRMLTFKFYMPKKA TELKHLQCLEEELKPLE EVLNLAQSKNFHLRPR DLISNINVIVLELKGSET TFMCEYADETATIVEFL NRWITFCQSIISTLT (SEQ ID NO: 3177) | Im- mune cells | 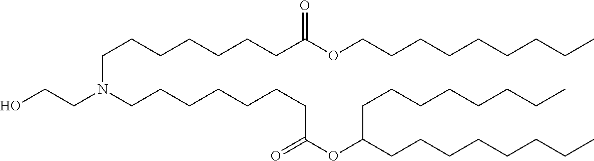 [SMILES: CCCCCCCCCOC(=O)CCCCCCCN(CCO)CCCCCCCC(=O)OC(CCCCCCCC)CCCCCCCC] |
| | | | (50 mol %) |
| | | | DSPC (10 mol %) |
| | | | Beta-sitosterol (28.5% mol %) |
| | | | Cholesterol (10 mol %) |
| | | | PEG DMG (1.5 mol %) |

In some embodiments, the expression sequence encodes a therapeutic protein. In some embodiments, the expression sequence encodes a cytokine, e.g., IL-12p70, IL-15, IL-2, IL-18, IL-21, IFN-α, IFN-β, IL-10, TGF-beta, IL-4, or IL-35, or a functional fragment thereof. In some embodiments, the expression sequence encodes an immune checkpoint inhibitor. In some embodiments, the expression sequence encodes an agonist (e.g., a TNFR family member such as CD137L, OX40L, ICOSL, LIGHT, or CD70). In some embodiments, the expression sequence encodes a chimeric antigen receptor. In some embodiments, the expression sequence encodes an inhibitory receptor agonist (e.g., PDL1, PDL2, Galectin-9, VISTA, B7H4, or MHCII) or inhibitory receptor (e.g., PD1, CTLA4, TIGIT, LAG3, or TIM3). In some embodiments, the expression sequence encodes an inhibitory receptor antagonist. In some embodiments, the expression sequence encodes one or more TCR chains (alpha and beta chains or gamma and delta chains). In some embodiments, the expression sequence encodes a secreted T cell or immune cell engager (e.g., a bispecific antibody such as BiTE, targeting, e.g., CD3, CD137, or CD28 and a tumor-expressed protein e.g., CD19, CD20, or BCMA etc.). In some embodiments, the expression sequence encodes a transcription factor (e.g., FOXP3, HELIOS, TOX1, or TOX2). In some embodiments, the expression sequence encodes an immunosuppressive enzyme (e.g., IDO or CD39/CD73). In some embodiments, the expression sequence encodes a GvHD (e.g., anti-HLA-A2 CAR-Tregs).

In some embodiments, the therapeutic protein is an antigen-binding protein, including but not limited to an antibody or an antigen-binding fragment thereof. An antigen-binding protein disclosed herein may be monoclonal or polyclonal. In some embodiments, the antigen-binding protein is monoclonal. In some embodiments, the antigen-binding protein is polyclonal. In particular embodiments, antigen-binding proteins of the present disclosure are human antibodies.

In some embodiments, the antibodies are intact immunoglobulin molecules, such as, e.g., a human antibody, as well as those portions of a humanized Ig molecule that contain the antigen-binding site (i.e., paratope) or a single heavy chain and a single light chain, including those portions known in the art such as Fab, Fab′, F(ab)′, F(ab′)2, Fd, scFv, a variable heavy domain, a variable light domain, a variable NAR domain, a single chain binding polypeptide, a dAb fragment, a nanobody, a VHH, and others also referred to as antigen-binding fragments. When constructing polynucleotides encoding an immunoglobulin molecule or fragments thereof, polynucleotides encoding variable regions or portions thereof are, in some embodiments, connected to, or otherwise joined to polynucleotides encoding one or more constant regions or portions thereof to produce any of the antibodies or fragments thereof described herein. Thus, in some embodiments, the antigen-binding fragment of any one of the antibodies described above is a Fab, Fab′, F(ab)′, F(ab′)2, Fd, scFv, a variable heavy domain, a variable light domain, a variable NAR domain, a dAb fragment, a nanobody, a VHH, a single chain binding polypeptide (e.g., a scFv with Fc portion) or any other functional fragment thereof as described herein.

In some embodiments, the antibodies are of any immunoglobulin class, and, therefore, in some embodiments, have a gamma, mu, alpha, delta, or epsilon heavy chain. In some embodiments, the gamma chain is gamma 1, gamma 2, gamma 3, or gamma 4. In some embodiments, the alpha chain is alpha 1 or alpha 2.

In some embodiments, an antibody of the present disclosure is an IgA immunoglobulin. In some embodiments, antibodies of the present disclosure are of any IgA subclass. In some embodiments, the antibody is IgA1. In some embodiments, the antibody is IgA2. In some embodiments, an antibody of the present disclosure is an IgD immunoglobulin. In some embodiments, an antibody of the present disclosure is an IgE immunoglobulin. In some embodiments, an antibody of the present disclosure is an IgG immunoglobulin. In some embodiments, antibodies of the present disclosure are of any IgG subclass. In some embodiments, the antibody is IgG1. In some embodiments, the antibody is IgG2. In some embodiments, the antibody is IgG3. In some embodiments, the antibody is IgG4. In some embodiments, an antibody of the present disclosure is an IgM immunoglobulin.

In some embodiments, antigen-binding proteins of the present disclosure comprise a variable light chain that is either kappa or lambda. In some embodiments, the lambda chain is of any subtype, including, e.g., lambda 1, lambda 2, lambda 3, and lambda 4. In some embodiments, the light chain is kappa.

In some embodiments, an antigen-binding protein of the present disclosure is monospecific. Exemplary monospecific antigen-binding proteins bind one epitope of a single antigen.

In some embodiments, an antigen-binding protein of the present disclosure is bispecific or multi-specific (e.g., tri-specific). Bispecific antigen-binding proteins have binding specificities for at least two different epitopes. Exemplary bispecific antigen-binding proteins, in some embodiments, bind to two different epitopes of a single antigen. Other such antigen-binding proteins, in some embodiments, combine a first antigen binding site with a binding site for a second antigen. In some embodiments, the bispecific antigen-binding proteins bind at least two different epitopes.

In some embodiments, an antigen-binding protein of the present disclosure has two or more valences, which are also referred to as multivalent. In some embodiments, an antigen-binding protein of the present disclosure is trispecific. In some embodiments, the antigen-binding proteins of the present disclosure are multivalent antibodies or fragments thereof with three or more antigen binding sites (e.g., tetravalent antibodies). In some embodiments, the multivalent antigen-binding protein comprises a dimerization domain and three or more antigen binding sites. In some embodiments, the dimerization domain comprises (or consists of) an Fc region or a hinge region. In this scenario, the antigen-binding protein will comprise an Fc region and three or more antigen binding sites amino-terminal to the Fc region. In some embodiments, the multivalent antigen-binding protein herein comprises about three to about eight, but preferably four, antigen binding sites. The multivalent antigen-binding protein comprises at least one polypeptide chain (and preferably two polypeptide chains), wherein the polypeptide chain(s) comprise two or more variable regions. For instance, the polypeptide chain(s) comprises VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. In some embodiments, the polypeptide chain(s) each independently comprise: V_{H}-C_{H}1-flexible linker-V_{H}-C_{H}1-Fc region chain; or V_{H}-C_{H}1-V_{H}-C_{H}1-Fc region chain. In some embodiments, the multivalent antigen-binding protein herein further comprises at least two (and preferably four) light chain variable region polypeptides. In some embodiments, the multivalent antigen-binding protein herein comprises from about two to about eight light chain variable region polypeptides. In some embodiments, the light chain variable region polypeptides described herein comprise a light chain variable region. In some embodiments, the light chain variable region polypeptides described herein further comprise a CL domain.

In some embodiments, an antigen-binding protein of the present disclosure is an SMIP or binding domain immunoglobulin fusion protein specific for the target protein. These constructs are single-chain polypeptides comprising antigen-binding domains fused to immunoglobulin domains necessary to carry out antibody effector functions.

In some embodiments, an antigen-binding protein of the present disclosure comprises a single chain binding polypeptide having a heavy chain variable region, and/or a light chain variable region which binds an epitope disclosed herein and has, optionally, an immunoglobulin Fc region. Such a molecule is a single chain variable fragment (scFv) optionally having effector function or increased half-life through the presence of the immunoglobulin Fc region.

As noted above, the disclosure further provides antibody fragments. In certain circumstances, there are advantages of using antibody fragments, rather than whole antibodies. For example, the smaller size of the fragments allows for rapid clearance, and leads to improved access to certain tissues, such as organs (e.g., lung, kidney, liver, or heart). Examples of antibody fragments include: Fab, F(ab′), F(ab′)₂, and Fv fragments, diabodies, linear antibodies, single-chain antibodies, and multispecific antibodies formed from antibody fragments.

The antigen-binding protein of choice can be a single chain Fv fragment (scFv). Fv and sFv are the only species with intact combining sites that are devoid of constant regions. Thus, they are suitable for reduced nonspecific binding during in vivo use. sFv fusion proteins can be constructed to yield fusion of an effector protein at either the amino or the carboxy terminus of an sFv. The antigen-binding protein fragment can also be a “linear antibody.” In some embodiments, such linear antibody fragments are monospecific or bispecific.

In some embodiments, a polynucleotide encodes a protein that is made up of subunits that are encoded by more than one gene. For example, the protein may be a heterodimer, wherein each chain or subunit of the protein is encoded by a separate gene. It is possible that more than one circRNA molecule is delivered in the transfer vehicle and each circRNA encodes a separate subunit of the protein. Alternatively, a single circRNA may be engineered to encode more than one subunit. In certain embodiments, separate circRNA molecules encoding the individual subunits may be administered in separate transfer vehicles.

### A. Antigen-Recognition Receptors

a. Chimeric Antigen Receptors (CARS)

In some embodiments, a provided RNA polynucleotide encodes one or more chimeric antigen receptors (CARs or CAR-Ts). CARs are genetically-engineered receptors. These engineered receptors may be inserted into and expressed by immune cells, including T cells via circular RNA as described herein. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell. In some embodiments, the CAR encoded by the polynucleotide comprises (i) an antigen-binding molecule that specifically binds to a target antigen, (ii) a hinge domain, a transmembrane domain, and an intracellular domain, and (iii) an activating domain.

In some embodiments, an orientation of the CARs in accordance with the disclosure comprises an antigen binding domain (such as an scFv) in tandem with a costimulatory domain and an activating domain. The costimulatory domain may comprise one or more of an extracellular portion, a transmembrane portion, and an intracellular portion. In other embodiments, multiple costimulatory domains may be utilized in tandem.

i. Antigen Binding Domain

CARs may be engineered to bind to an antigen (such as a cell-surface antigen) by incorporating an antigen binding molecule that interacts with that targeted antigen. In some embodiments, the antigen binding molecule is an antibody fragment thereof, e.g., one or more single chain antibody fragment (scFv). An scFv is a single chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45: 131-136. An scFv retains the parent antibody's ability to specifically interact with target antigen. scFvs are useful in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797. It will be appreciated that the antigen binding molecule is typically contained within the extracellular portion of the CAR such that it is capable of recognizing and binding to the antigen of interest. Bispecific and multispecific CARs are contemplated within the scope of the invention, with specificity to more than one target of interest.

In some embodiments, the antigen binding molecule comprises a single chain, wherein the heavy chain variable region and the light chain variable region are connected by a linker. In some embodiments, the VH is located at the N terminus of the linker and the VL is located at the C terminus of the linker. In other embodiments, the VL is located at the N terminus of the linker and the VH is located at the C terminus of the linker. In some embodiments, the linker comprises at least about 5, at least about 8, at least about 10, at least about 13, at least about 15, at least about 18, at least about 20, at least about 25, at least about 30, at least about 35, at least about 40, at least about 45, at least about 50, at least about 60, at least about 70, at least about 80, at least about 90, or at least about 100 amino acids.

In some embodiments, the antigen binding molecule comprises a nanobody. In some embodiments, the antigen binding molecule comprises a DARPin. In some embodiments, the antigen binding molecule comprises an anticalin or other synthetic protein capable of specific binding to target protein.

In some embodiments, the CAR comprises an antigen binding domain specific for an antigen selected from the group CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GaINAca-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, HER2, HER3, Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Ab1) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WT1), Cancer/testis antigen 1 (NY-ESO-1), Cancer/testis antigen 2 (LAGE-1a), MAGE family members (including MAGE-A1, MAGE-A3 and MAGE-A4), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, survivin, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLECI2A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), MUC16, 5T4, 8H9, αvβθ integrin, αvβ6 integrin, alphafetoprotein (AFP), B7-H6, ca-125, CA9, CD44, CD44v7/8, CD52, E-cadherin, EMA (epithelial membrane antigen), epithelial glycoprotein-2 (EGP-2), epithelial glycoprotein-40 (EGP-40), ErbB4, epithelial tumor antigen (ETA), folate binding protein (FBP), kinase insert domain receptor (KDR), k-light chain, L1 cell adhesion molecule, MUC18, NKG2D, oncofetal antigen (h5T4), tumor/testis-antigen 1B, GAGE, GAGE-1, BAGE, SCP-1, CTZ9, SAGE, CAGE, CT10, MART-1, immunoglobulin lambda-like polypeptide 1 (IGLL1), Hepatitis B Surface Antigen Binding Protein (HBsAg), viral capsid antigen (VCA), early antigen (EA), EBV nuclear antigen (EBNA), HHV-6 p41 early antigen, HHV-6B U94 latent antigen, HHV-6B p98 late antigen, cytomegalovirus (CMV) antigen, large T antigen, small T antigen, adenovirus antigen, respiratory syncytial virus (RSV) antigen, haemagglutinin (HA), neuraminidase (NA), parainfluenza type 1 antigen, parainfluenza type 2 antigen, parainfluenza type 3 antigen, parainfluenza type 4 antigen, Human Metapneumovirus (HMPV) antigen, hepatitis C virus (HCV) core antigen, HIV p24 antigen, human T cell lymphotrophic virus (HTLV-1) antigen, Merkel cell polyoma virus small T antigen, Merkel cell polyoma virus large T antigen, Kaposi sarcoma-associated herpesvirus (KSHV) lytic nuclear antigen and KSHV latent nuclear antigen. In some embodiments, an antigen binding domain comprises an amino acid sequence selected from SEQ ID NOs: 3162-3176.

ii. Hinge/Spacer Domain

In some embodiments, a CAR of the instant disclosure comprises a hinge or spacer domain. In some embodiments, the hinge/spacer domain may comprise a truncated hinge/spacer domain (THD) the THD domain is a truncated version of a complete hinge/spacer domain (“CHD”). In some embodiments, an extracellular domain is from or derived from (e.g., comprises all or a fragment of) ErbB2, glycophorin A (GpA), CD2, CD3 delta, CD3 epsilon, CD3 gamma, CD4, CD7, CD8a, CD8[T CD11a (IT GAL), CD11b (IT GAM), CD11c (ITGAX), CD11d (IT GAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (0X40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRT AM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof. A hinge or spacer domain may be derived either from a natural or from a synthetic source.

In some embodiments, a hinge or spacer domain is positioned between an antigen binding molecule (e.g., an scFv) and a transmembrane domain. In this orientation, the hinge/spacer domain provides distance between the antigen binding molecule and the surface of a cell membrane on which the CAR is expressed. In some embodiments, a hinge or spacer domain is from or derived from an immunoglobulin. In some embodiments, a hinge or spacer domain is selected from the hinge/spacer regions of IgG1, IgG2, IgG3, IgG4, IgA, IgD, IgE, IgM, or a fragment thereof. In some embodiments, a hinge or spacer domain comprises, is from, or is derived from the hinge/spacer region of CD8 alpha. In some embodiments, a hinge or spacer domain comprises, is from, or is derived from the hinge/spacer region of CD28. In some embodiments, a hinge or spacer domain comprises a fragment of the hinge/spacer region of CD8 alpha or a fragment of the hinge/spacer region of CD28, wherein the fragment is anything less than the whole hinge/spacer region. In some embodiments, the fragment of the CD8 alpha hinge/spacer region or the fragment of the CD28 hinge/spacer region comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of the CD8 alpha hinge/spacer region, or of the CD28 hinge/spacer region.

iii. Transmembrane Domain

The CAR of the present disclosure may further comprise a transmembrane domain and/or an intracellular signaling domain. The transmembrane domain may be designed to be fused to the extracellular domain of the CAR. It may similarly be fused to the intracellular domain of the CAR. In some embodiments, the transmembrane domain that naturally is associated with one of the domains in a CAR is used. In some instances, the transmembrane domain may be selected or modified (e.g., by an amino acid substitution) to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein.

Transmembrane regions may be derived from (i.e. comprise) a receptor tyrosine kinase (e.g., ErbB2), glycophorin A (GpA), 4-1BB/CD137, activating NK cell receptors, an immunoglobulin protein, B7-H3, BAFFR, BFAME (SEAMF8), BTEA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (EIGHTR), IA4, ICAM-1, ICAM-1, Ig alpha (CD79a), IE-2R beta, IE-2R gamma, IE-7R alpha, inducible T cell costimulator (ICOS), integrins, ITGA4, ITGA4, ITGA6, IT GAD, ITGAE, ITGAE, IT GAM, ITGAX, ITGB2, ITGB7, ITGB1, KIRDS2, EAT, LFA-1, LFA-1, a ligand that specifically binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Lyl08), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

In some embodiments, suitable intracellular signaling domain include, but are not limited to, activating Macrophage/Myeloid cell receptors CSFR1, MYD88, CD14, TIE2, TLR4, CR3, CD64, TREM2, DAP10, DAP12, CD169, DECTIN1, CD206, CD47, CD163, CD36, MARCO, TIM4, MERTK, F4/80, CD91, C1QR, LOX-1, CD68, SRA, BAI-1, ABCA7, CD36, CD31, Lactoferrin, or a fragment, truncation, or combination thereof.

In some embodiments, a receptor tyrosine kinase may be derived from (e.g., comprise) Insulin receptor (InsR), Insulin-like growth factor 1 receptor (IGF1R), Insulin receptor-related receptor (IRR), platelet derived growth factor receptor alpha (PDGFRa), platelet derived growth factor receptor beta (PDGFRfi). KIT proto-oncogene receptor tyrosine kinase (Kit), colony stimulating factor 1 receptor (CSFR), fms related tyrosine kinase 3 (FLT3), fms related tyrosine kinase 1 (VEGFR-1), kinase insert domain receptor (VEGFR-2), fms related tyrosine kinase 4 (VEGFR-3), fibroblast growth factor receptor 1 (FGFR1), fibroblast growth factor receptor 2 (FGFR2), fibroblast growth factor receptor 3 (FGFR3), fibroblast growth factor receptor 4 (FGFR4), protein tyrosine kinase 7 (CCK4), neurotrophic receptor tyrosine kinase 1 (trkA), neurotrophic receptor tyrosine kinase 2 (trkB), neurotrophic receptor tyrosine kinase 3 (trkC), receptor tyrosine kinase like orphan receptor 1 (ROR1), receptor tyrosine kinase like orphan receptor 2 (ROR2), muscle associated receptor tyrosine kinase (MuSK), MET proto-oncogene, receptor tyrosine kinase (MET), macrophage stimulating 1 receptor (Ron), AXL receptor tyrosine kinase (Axl), TYR03 protein tyrosine kinase (Tyro3), MER proto-oncogene, tyrosine kinase (Mer), tyrosine kinase with immunoglobulin like and EGF like domains 1 (TIE1), TEK receptor tyrosine kinase (TIE2), EPH receptor A1 (EphA1), EPH receptor A2 (EphA2), (EPH receptor A3) EphA3, EPH receptor A4 (EphA4), EPH receptor A5 (EphA5), EPH receptor A6 (EphA6), EPH receptor A7 (EphA7), EPH receptor A8 (EphA8), EPH receptor A10 (EphAlO), EPH receptor B1 (EphB1), EPH receptor B2 (EphB2), EPH receptor B3 (EphB3), EPH receptor B4 (EphB4), EPH receptor B6 (EphB6), ret proto oncogene (Ret), receptor-like tyrosine kinase (RYK), discoidin domain receptor tyrosine kinase 1 (DDR1), discoidin domain receptor tyrosine kinase 2 (DDR2), c-ros oncogene 1, receptor tyrosine kinase (ROS), apoptosis associated tyrosine kinase (Lmr1), lemur tyrosine kinase 2 (Lmr2), lemur tyrosine kinase 3 (Lmr3), leukocyte receptor tyrosine kinase (LTK), ALK receptor tyrosine kinase (ALK), or serine/threonine/tyrosine kinase 1 (STYK1).

iv. Costimulatory Domain

In certain embodiments, the CAR comprises a costimulatory domain. In some embodiments, the costimulatory domain comprises 4-1BB (CD137), CD28, or both, and/or an intracellular T cell signaling domain. In a preferred embodiment, the costimulatory domain is human CD28, human 4-1BB, or both, and the intracellular T cell signaling domain is human CD3 zeta (ζ). 4-1BB, CD28, CD3 zeta may comprise less than the whole 4-1BB, CD28 or CD3 zeta, respectively. Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Amur. Rev. Pharmacol. Toxicol. 56:59-83 (2016).

v. Intracellular Signaling Domain

The intracellular (signaling) domain of the engineered T-cells disclosed herein may provide signaling to an activating domain, which then activates at least one of the normal effector functions of the immune cell. Effector function of a T-cell, for example, may be cytolytic activity or helper activity including the secretion of cytokines.

In some embodiments, suitable intracellular signaling domain include (e.g., comprise), but are not limited to 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), integrins, ITGA4, ITGA6, ITGAD, ITGAE, ITGAL, ITGAM, ITGAX, ITGB2, ITGB7, ITGB1, KIRDS2, LAT, LFA-1, ligand that specifically binds with CD83, LIGHT, LTBR, Ly9 (CD229), Lyl08, lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

CD3 is an element of the T-cell receptor on native T-cells, and has been shown to be an important intracellular activating element in CARs. In some embodiments, the CD3 is CD3 zeta. In some embodiments, the activating domain comprises an amino acid sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a polypeptide sequence selected from SEQ ID NOs: 3162-3176.

b. T-Cell Receptors (TCR)

In some embodiments, a provided circular RNA polynucleotide encodes a T-cell receptor. TCRs are described using the International Immunogenetics (IMGT) TCR nomenclature, and links to the IMGT public database of TCR sequences. Native alpha-beta heterodimeric TCRs have an alpha chain and a beta chain. Broadly, each chain may comprise variable, joining and constant regions, and the beta chain also usually contains a short diversity region between the variable and joining regions, but this diversity region is often considered as part of the joining region. Each variable region may comprise three CDRs (Complementarity Determining Regions) embedded in a framework sequence, one being the hypervariable region named CDR3. There are several types of alpha chain variable (Vα) regions and several types of beta chain variable (Vβ) regions distinguished by their framework, CDR1 and CDR2 sequences, and by a partly defined CDR3 sequence. The Va types are referred to in IMGT nomenclature by a unique TRAV number. Thus “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. In the same way, “TRBV5-1” defines a TCR Vβ region having unique framework and CDR1 and CDR2 sequences, but with only a partly defined CDR3 sequence.

The joining regions of the TCR are similarly defined by the unique IMGT TRAJ and TRBJ nomenclature, and the constant regions by the IMGT TRAC and TRBC nomenclature.

The beta chain diversity region is referred to in IMGT nomenclature by the abbreviation TRBD, and, as mentioned, the concatenated TRBD/TRBJ regions are often considered together as the joining region.

The unique sequences defined by the IMGT nomenclature are widely known and accessible to those working in the TCR field. For example, they can be found in the IMGT public database. The “T cell Receptor Factsbook”, (2001) LeFranc and LeFranc, Academic Press, ISBN 0-12-441352-8 also discloses sequences defined by the IMGT nomenclature, but because of its publication date and consequent time-lag, the information therein sometimes needs to be confirmed by reference to the IMGT database.

Native TCRs exist in heterodimeric αβ or γδ forms. However, recombinant TCRs consisting of aa or RR homodimers have previously been shown to bind to peptide MHC molecules. Therefore, the TCR of the invention may be a heterodimeric αβ TCR or may be an aa or RR homodimeric TCR.

For use in adoptive therapy, an αβ heterodimeric TCR may, for example, be transfected as full length chains having both cytoplasmic and transmembrane domains. In certain embodiments TCRs of the invention may have an introduced disulfide bond between residues of the respective constant domains, as described, for example, in WO 2006/000830.

TCRs of the invention, particularly alpha-beta heterodimeric TCRs, may comprise an alpha chain TRAC constant domain sequence and/or a beta chain TRBC1 or TRBC2 constant domain sequence. The alpha and beta chain constant domain sequences may be modified by truncation or substitution to delete the native disulfide bond between Cys4 of exon 2 of TRAC and Cys2 of exon 2 of TRBC1 or TRBC2. The alpha and/or beta chain constant domain sequence(s) may also be modified by substitution of cysteine residues for Thr 48 of TRAC and Ser 57 of TRBC1 or TRBC2, the said cysteines forming a disulfide bond between the alpha and beta constant domains of the TCR.

Binding affinity (inversely proportional to the equilibrium constant K_{D}) and binding half-life (expressed as T½) can be Determined by any Appropriate Method. It Will be appreciated that doubling the affinity of a TCR results in halving the K_{D}. T½ is calculated as ln 2 divided by the off-rate (koff). So doubling of T½ results in a halving in koff. K_{D} and koff values for TCRs are usually measured for soluble forms of the TCR, i.e. those forms which are truncated to remove cytoplasmic and transmembrane domain residues. Therefore it is to be understood that a given TCR has an improved binding affinity for, and/or a binding half-life for the parental TCR if a soluble form of that TCR has the said characteristics. Preferably the binding affinity or binding half-life of a given TCR is measured several times, for example 3 or more times, using the same assay protocol, and an average of the results is taken.

Since the TCRs of the invention have utility in adoptive therapy, the invention includes a non-naturally occurring and/or purified and/or or engineered cell, especially a T-cell, presenting a TCR of the invention. There are a number of methods suitable for the transfection of T-cells with nucleic acid (such as DNA, cDNA or RNA) encoding the TCRs of the invention (see for example Robbins et al., (2008) J Immunol. 180: 6116-6131). T-cells expressing the TCRs of the invention will be suitable for use in adoptive therapy-based treatment of cancers such as those of the pancreas and liver. As will be known to those skilled in the art, there are a number of suitable methods by which adoptive therapy can be carried out (see for example Rosenberg et al., (2008) Nat Rev Cancer 8(4): 299-308).

As is well-known in the art TCRs of the invention may be subject to post-translational modifications when expressed by transfected cells. Glycosylation is one such modification, which may comprise the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e., oligosaccharide type, covalent linkage and total number of attachments) can influence protein function. Therefore, when producing recombinant proteins, controlling glycosylation is often desirable. Glycosylation of transfected TCRs may be controlled by mutations of the transfected gene (Kuball J et al. (2009), J Exp Med 206(2):463-475). Such mutations are also encompassed in this invention.

A TCR may be specific for an antigen in the group MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (AGE-B4), tyrosinase, brain glycogen phosphorylase, Melan-A, MAGE-C1, MAGE-C2, NY-ESO-1, LAGE-1, SSX-1, SSX-2(HOM-MEL-40), SSX-1, SSX-4, SSX-5, SCP-1, CT-7, alpha-actinin-4, Bcr-Ab1 fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pm1-RARa fusion protein, PTPRK, K-ras, N-ras, Triosephosphate isomeras, GnTV, Herv-K-mel, Lage-1, Mage-C₂, NA-88, Lage-2, SP17, and TRP2-Int2, (MART-I), gp100 (Pmel 17), TRP-1, TRP-2, MAGE-1, MAGE-3, pi5(58), CEA, NY-ESO (LAGE), SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, beta-catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAGi6, TA-90 (Mac-2 binding protein\cyclophilin C-associated protein), TAAL6, TAG72, TLP, and TPS.

c. B-Cell Receptors (BCR)

In some embodiments, a provided circular RNA polynucleotide encodes one or more B-cell receptors (BCRs). BCRs (or B-cell antigen receptors) are immunoglobulin molecules that form a type I transmembrane protein on the surface of a B cell. A BCR is capable of transmitting activating signal into a B cell following recognition of a specific antigen. Prior to binding of a B cell to an antigen, the BCR will remain in an unstimulated or “resting” stage. Binding of an antigen to a BCR leads to signaling that initiates a humoral immune response.

A BCR is expressed by mature B cells. These B cells work with immunoglobulins (Igs) in recognizing and tagging pathogens. The typical BCR comprises a membrane-bound immunoglobulin (e.g., mIgA, mIgD, mIgE, mIgG, and mIgM), along with associated and Igα/Igβ (CD79a/CD79b) heterodimers (α/β). These membrane-bound immunoglobulins are tetramers consisting of two identical heavy and two light chains. Within the BCR, the membrane bound immunoglobulins is capable of responding to antigen binding by signal transmission across the plasma membrane leading to B cell activation and consequently clonal expansion and specific antibody production (Friess M et al. (2018), Front. Immunol. 2947(9)). The Igα/Igβ heterodimers is responsible for transducing signals to the cell interior.

A Igα/Igβ heterodimer signaling relies on the presence of immunoreceptor tyrosine-based activation motifs (ITAMs) located on each of the cytosolic tails of the heterodimers. ITAMs comprise two tyrosine residues separated by 9-12 amino acids (e.g., tyrosine, leucine, and/or valine). Upon binding of an antigen, the tyrosine of the BCR's ITAMs become phosphorylated by Src-family tyrosine kinases Blk, Fyn, or Lyn (Janeway C et al., Immunobiology: The Immune System in Health and Disease (Garland Science, 5th ed. 2001)).

d. Other Chimeric Proteins

In addition to the chimeric proteins provided above, the circular RNA polynucleotide may encode for a various number of other chimeric proteins available in the art. The chimeric proteins may include recombinant fusion proteins, chimeric mutant protein, or other fusion proteins.

### B. Immune Modulatory Ligands

In some embodiments, the circular RNA polynucleotide encodes for an immune modulatory ligand. In certain embodiments, the immune modulatory ligand may be immunostimulatory; while in other embodiments, the immune modulatory ligand may be immunosuppressive.

a. Cytokines: Interferon, Chemokines, Interleukins, Growth Factor & Others

In some embodiments, the circular RNA polynucleotide encodes for a cytokine. In some embodiments, the cytokine comprises a chemokine, interferon, interleukin, lymphokine, and tumor necrosis factor. Chemokines are chemotactic cytokine produced by a variety of cell types in acute and chronic inflammation that mobilizes and activates white blood cells. An interferon comprises a family of secreted α-helical cytokines induced in response to specific extracellular molecules through stimulation of TLRs (Borden, Molecular Basis of Cancer (Fourth Edition) 2015). Interleukins are cytokines expressed by leukocytes.

Descriptions and/or amino acid sequences of IL-2, IL-7, IL-10, IL-12, IL-15, IL-18, IL-27β, IFNγ, and/or TGFβ1 are provided herein and at the www.uniprot.org database at accession numbers: P60568 (IL-2), P29459 (IL-12A), P29460 (IL-12B), P13232 (IL-7), P22301 (IL-10), P40933 (IL-15), Q14116 (IL-18), Q14213 (IL-27β), P01579 (IFNγ), and/or P01137 (TGFβ1).

### C. Transcription Factors

Regulatory T-cells (Treg) are important in maintaining homeostasis, controlling the magnitude and duration of the inflammatory response, and in preventing autoimmune and allergic responses.

In general, Tregs are thought to be mainly involved in suppressing immune responses, functioning in part as a “self-check” for the immune system to prevent excessive reactions. In particular, Tregs are involved in maintaining tolerance to self-antigens, harmless agents such as pollen or food, and abrogating autoimmune disease.

Tregs are found throughout the body including, without limitation, the gut, skin, lung, and liver. Additionally, Treg cells may also be found in certain compartments of the body that are not directly exposed to the external environment such as the spleen, lymph nodes, and even adipose tissue. Each of these Treg cell populations is known or suspected to have one or more unique features and additional information may be found in Lehtimaki and Lahesmaa, Regulatory T-cells control immune responses through their non-redundant tissue specific features, 2013, FRONTIERS IN IMMUNOL., 4(294): 1-10, the disclosure of which is hereby incorporated in its entirety.

Typically, Tregs are known to require TGF-β and IL-2 for proper activation and development. Tregs, expressing abundant amounts of the IL-2 receptor (IL-2R), are reliant on IL-2 produced by activated T-cells. Tregs are known to produce both IL-10 and TGF-β, both potent immune suppressive cytokines. Additionally, Tregs are known to inhibit the ability of antigen presenting cells (APCs) to stimulate T-cells. One proposed mechanism for APC inhibition is via CTLA-4, which is expressed by Foxp3+ Tregs. It is thought that CTLA-4 may bind to B7 molecules on APCs and either block these molecules or remove them by causing internalization resulting in reduced availability of B7 and an inability to provide adequate co-stimulation for immune responses. Additional discussion regarding the origin, differentiation and function of Tregs may be found in Dhamne et al., Peripheral and thymic Foxp3+ regulatory T-cells in search of origin, distinction, and function, 2013, Frontiers in Immunol., 4 (253): 1-11, the disclosure of which is hereby incorporated in its entirety.

### D. Checkpoint Inhibitors & Agonists

As provided herein, in certain embodiments, the coding element of the circular RNA encodes for one or more checkpoint inhibitors or agonists.

In some embodiments, the immune checkpoint inhibitor is an inhibitor of Programmed Death-Ligand 1 (PD-L1, also known as B7-H1, CD274), Programmed Death 1 (PD-1), CTLA-4, PD-L2 (B7-DC, CD273), LAG3, TIM3, 2B4, A2aR, B7H1, B7H3, B7H4, BTLA, CD2, CD27, CD28, CD30, CD40, CD70, CD80, CD86, CD137, CD160, CD226, CD276, DR3, GAL9, GITR, HAVCR2, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGHT, VISTA, VTCN1, or any combinations thereof. In some embodiments, the immune checkpoint inhibitor is an inhibitor of IDO1, CTLA4, PD-1, LAG3, PD-L1, TIM3, or combinations thereof. In some embodiments, the immune checkpoint inhibitor is an inhibitor of PD-L1. In some embodiments, the immune checkpoint inhibitor is an inhibitor of PD-1. In some embodiments, the immune checkpoint inhibitor is an inhibitor of CTLA-4. In some embodiments, the immune checkpoint inhibitor is an inhibitor of LAG3. In some embodiments, the immune checkpoint inhibitor is an inhibitor of TIM3. In some embodiments, the immune checkpoint inhibitor is an inhibitor of IDO1.

As described herein, at least in one aspect, the invention encompasses the use of immune checkpoint antagonists. Such immune checkpoint antagonists include antagonists of immune checkpoint molecules such as Cytotoxic T-Lymphocyte Antigen 4 (CTLA-4), Programmed Cell Death Protein 1 (PD-1), Programmed Death-Ligand 1 (PDL-1), Lymphocyte-activation gene 3 (LAG-3), and T cell immunoglobulin and mucin domain 3 (TIM-3). An antagonist of CTLA-4, PD-1, PDL-1, LAG-3, or TIM-3 interferes with CTLA-4, PD-1, PDL-1, LAG-3, or TIM-3 function, respectively. Such antagonists of CTLA-4, PD-1, PDL-1, LAG-3, and TIM-3 can include antibodies which specifically bind to CTLA-4, PD-1, PDL-1, LAG-3, and TIM-3, respectively and inhibit and/or block biological activity and function.

### E. Others

In some embodiments, the payload encoded within one or more of the coding elements is a hormone, FC fusion protein, anticoagulant, blood clotting factor, protein associated with deficiencies and genetic disease, a chaperone protein, an antimicrobial protein, an enzyme (e.g., metabolic enzyme), a structural protein (e.g., a channel or nuclear pore protein), protein variant, small molecule, antibody, nanobody, an engineered non-body antibody, or a combination thereof.

### 4. Additional Accessory Elements (Sequence Elements)

As described in this invention, the polynucleotide (e.g., circular RNA polynucleotide, linear RNA polynucleotide, and/or DNA template) may further comprise of accessory elements. In certain embodiments, these accessory elements may be included within the sequences of the circular RNA, linear RNA polynucleotide and/or DNA template for enhancing circularization, translation or both. Accessory elements are sequences, in certain embodiments that are located with specificity between or within the enhanced intron elements, enhanced exon elements, or core functional element of the respective polynucleotide. As an example, but not intended to be limiting, an accessory element includes, a IRES transacting factor region, a miRNA binding site, a restriction site, an RNA editing region, a structural or sequence element, a granule site, a zip code element, an RNA trafficking element or another specialized sequence as found in the art that enhances promotes circularization and/or translation of the protein encoded within the circular RNA polynucleotide.

### A. IRES Transacting Factors

In certain embodiments, the accessory element comprises an IRES transacting factor (ITAF) region. In some embodiments, the IRES transacting factor region modulates the initiation of translation through binding to PCBP1-PCBP4 (polyC binding protein), PABP1 (polyA binding protein), PTB (polypyrimidine tract binding), Argonaute protein family, HNRNPK (Heterogeneous nuclear ribonucleoprotein K protein), or La protein. In some embodiments, the IRES transacting factor region comprises a polyA, polyC, polyAC, or polypyrimidine track.

In some embodiments, the ITAF region is located within the core functional element. In some embodiments, the ITAF region is located within the TIE.

### B. miRNA Binding Sites

In certain embodiments, the accessory element comprises a miRNA binding site. In some embodiments the miRNA binding site is located within the 5′ enhanced intron element, 5′ enhanced exon element, core functional element, 3′ enhanced exon element, and/or 3′ enhanced intron element.

In some embodiments, wherein the miRNA binding site is located within the spacer within the enhanced intron element or enhanced exon element. In certain embodiments, the miRNA binding site comprises the entire spacer regions.

In some embodiments, the 5′ enhanced intron element and 3′ enhanced intron elements each comprise identical miRNA binding sites. In another embodiment, the miRNA binding site of the 5′ enhanced intron element comprises a different, in length or nucleotides, miRNA binding site than the 3′ enhanced intron element. In one embodiment, the 5′ enhanced exon element and 3′ enhanced exon element comprise identical miRNA binding sites. In other embodiments, the 5′ enhanced exon element and 3′ enhanced exon element comprises different, in length or nucleotides, miRNA binding sites.

In some embodiments, the miRNA binding sites are located adjacent to each other within the circular RNA polynucleotide, linear RNA polynucleotide precursor, and/or DNA template. In certain embodiments, the first nucleotide of one of the miRNA binding sites follows the first nucleotide last nucleotide of the second miRNA binding site.

In some embodiments, the miRNA binding site is located within a translation initiation element (TIE) of a core functional element. In one embodiment, the miRNA binding site is located before, trailing or within an internal ribosome entry site (IRES). In another embodiment, the miRNA binding site is located before, trailing, or within an aptamer complex.

Incorporation of miRNA sequences within a circular RNA molecule can permit tissue-specific expression of a coding sequence within a core functional element. For example, in a circular RNA intended to express a protein in immune cells, miRNA binding sequences resulting in expression suppression in tissues such as the liver or kidney may be desired. Such miRNA binding sequences may be selected based on the cell or tissue expression of miRNAs.

The unique sequences defined by the miRNA nomenclature are widely known and accessible to those working in the microRNA field. For example, they can be found in the miRDB public database.

### 5. Production of Polynucleotides

The DNA templates provided herein can be made using standard techniques of molecular biology. For example, the various elements of the vectors provided herein can be obtained using recombinant methods, such as by screening cDNA and genomic libraries from cells, or by deriving the polynucleotides from a DNA template known to include the same.

The various elements of the DNA template provided herein can also be produced synthetically, rather than cloned, based on the known sequences. The complete sequence can be assembled from overlapping oligonucleotides prepared by standard methods and assembled into the complete sequence. See, e.g., Edge, Nature (1981) 292:756; Nambair et al., Science (1984) 223: 1299; and Jay et al., J. Biol. Chem. (1984) 259:631 1.

Thus, particular nucleotide sequences can be obtained from DNA template harboring the desired sequences or synthesized completely, or in part, using various oligonucleotide synthesis techniques known in the art, such as site-directed mutagenesis and polymerase chain reaction (PCR) techniques where appropriate. One method of obtaining nucleotide sequences encoding the desired DNA template elements is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54:75-82), oligonucleotide directed mutagenesis of preexisting nucleotide regions (Riechmann et al., Nature (1988) 332:323-327 and Verhoeyen et al., Science (1988) 239: 1534-1536), and enzymatic filling-in of gapped oligonucleotides using T4 DNA polymerase (Queen et al., Proc. Natl. Acad. Sci. USA (1989) 86: 10029-10033) can be used.

The precursor RNA provided herein can be generated by incubating a DNA template provided herein under conditions permissive of transcription of the precursor RNA encoded by the DNA template. For example, in some embodiments a precursor RNA is synthesized by incubating a DNA template provided herein that comprises an RNA polymerase promoter upstream of its 5′ duplex sequence and/or expression sequences with a compatible RNA polymerase enzyme under conditions permissive of in vitro transcription. In some embodiments, the DNA template is incubated inside of a cell by a bacteriophage RNA polymerase or in the nucleus of a cell by host RNA polymerase II.

In certain embodiments, provided herein is a method of generating precursor RNA by performing in vitro transcription using a DNA template provided herein as a template (e.g., a vector provided herein with an RNA polymerase promoter positioned upstream of the 5′ duplex region).

In certain embodiments, the resulting precursor RNA can be used to generate circular RNA (e.g., a circular RNA polynucleotide provided herein) by incubating it in the presence of magnesium ions and guanosine nucleotide or nucleoside at a temperature at which RNA circularization occurs (e.g., between 20° C. and 60° C.).

Thus, in certain embodiments provided herein is a method of making circular RNA. In certain embodiments, the method comprises synthesizing precursor RNA by transcription (e.g., run-off transcription) using a vector provided herein (e.g., a 5′ enhanced intron element, a 5′ enhanced exon element, a core functional element, a 3′ enhanced exon element, and a 3′ enhanced intron element) as a template, and incubating the resulting precursor RNA in the presence of divalent cations (e.g., magnesium ions) and GTP such that it circularizes to form circular RNA. In some embodiments, the precursor RNA disclosed herein is capable of circularizing in the absence of magnesium ions and GTP and/or without the step of incubation with magnesium ions and GTP. It has been discovered that circular RNA has reduced immunogenicity relative to a corresponding mRNA, at least partially because the mRNA contains an immunogenic 5′ cap. When transcribing a DNA vector from certain promoters (e.g., a T7 promoter) to produce a precursor RNA, it is understood that the 5′ end of the precursor RNA is G. To reduce the immunogenicity of a circular RNA composition that contains a low level of contaminant linear mRNA, an excess of GMP relative to GTP can be provided during transcription such that most transcripts contain a 5′ GMP, which cannot be capped. Therefore, in some embodiments, transcription is carried out in the presence of an excess of GMP. In some embodiments, transcription is carried out where the ratio of GMP concentration to GTP concentration is within the range of about 3:1 to about 15:1, for example, about 3:1 to about 10:1, about 3:1 to about 5:1, about 3:1, about 4:1, or about 5:1.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA. In some embodiments, purification of circular RNA comprises an affinity-purification or negative selection method described herein. In some embodiments, purification of circular RNA comprises separation of linear RNA from circular RNA using oligonucleotides that are complementary to a sequence in the linear RNA but are not complementary to a sequence in the circular RNA. In some embodiments, a purified composition is less immunogenic than an unpurified composition. In some embodiments, immune cells exposed to a purified composition produce less TNFα, RIG-I, IL-2, IL-6, IFNγ, and/or a type 1 interferon, e.g., IFN-β1, than immune cells exposed to an unpurified composition.

Described herein is a method of evaluating RNA translatability using polysome profiling. Polysome profiling involves the analysis of ribosome-free and polysome-bound RNAs and may be used to determine translatability of an RNA molecule (see, e.g., Leppek et al., Nature Commun. (2022), 13, 1536). Polysomes are an aggregate of ribosomes that are in the process of actively translating RNA into protein and have a different molecular weight as compared to unbound ribosomes (i.e., non-translating ribosomes that are not bound to an RNA molecule). Given that there are different molecular weights between bound polysomes and unbound ribosomes, ribosome fractionation can be conducted for the bound polysomes (associated with actively translating RNAs) and unbound (non-translating RNAs) molecules. In some embodiments, separation and/or fractionation of the polysomes may be performed using a sucrose gradient.

Polysomes having RNAs with multiple bound ribosomes are an indication of higher translational capacity. By contrast, free RNAs that are not bound by any ribosomes or ribosomal subunits are an indication of lower or no translational capacity. In some embodiments, the RNA sequences (e.g., circular RNA), following cellular introduction and isolation, may be bound by two or more ribosomes resulting in a sucrose fractionation correlating to the number of bound ribosomes. In certain embodiments, the RNA sequences may be bound by a single ribosome resulting in a correlative sucrose fraction. In some embodiments, the RNA sequences may be bound by the 60s ribosomal subunit resulting in a correlative sucrose fraction. In other embodiments, the RNA sequences may be bound by the 40s ribosomal subunit resulting in a correlative sucrose fraction. Alternatively, in some embodiments, the RNA sequences may not be bound by any ribosomes or subunits resulting in a correlative sucrose fraction.

In certain embodiments, RNA polysome loading (i.e., the process by which the RNA of interest is loaded onto ribosome) can be evaluated in a cell-free expression (CFE) environment. CFE is a system by which polynucleotides can be evaluated for their transcription and translational capacity via an in-vitro environment. Such experiments may comprise introducing a DNA template into a mix of a cytoplasmic extract consisting of the cellular products necessary for transcription and translation (see, e.g., Garenne et al., Nat. Rev. Methods Primers (2021), 1, 49). From this system, the polysomes may then be collected and evaluated via the polysome profiling methods described herein.

In some embodiments, a pool of the RNA of interest (e.g., circular RNA) is transfected into a collection of cells (e.g., mammalian cells) instead of a CFE environment. In some embodiments, the cells undergo cell lysis to release the cytoplasmic polysomes that are then extracted for polysome profiling analysis. In some embodiments, following cell lysis, the lysed cells and their RNA content are added to a sucrose gradient and centrifuged to separate the RNA molecules based on their ribosomal load (see, e.g., bChass6 et al., Nucleic Acids Res. (2017), 45(3):e15). The sucrose gradients are then fractionated by pumping the gradients into a fractional collection instrument and collected as individual sucrose/polysome fractions. In some embodiments, the nature of these fractions can be defined by volume and/or time. A set referential ‘ladder’ RNA sequences are added to the individual fractions at known concentrations to aid in quantifying and for comparative purposes against the original pool of RNA molecules. The RNA may be then extracted from the individual sucrose gradient fractions for sequencing and analysis (e.g., RNA stability).

In some embodiments, polysome profiling may be used to determine translatability of one or more RNA molecule. In some embodiments, the RNA molecules analyzed through polysome profiling comprise a circular RNA and/or linear RNA. In some embodiments, the RNA (e.g., circular RNA) comprises a translation initiation element (TIE) described herein. In some embodiments, the polysome profiling may be used to select and optimize translatability and/or expression in the RNA molecule. In some embodiments, a TIE capable of high translation efficiency, e.g., in a circular RNA disclosed herein, refers to high ribosome initiation, load, and/or recruitment, e.g., de novo ribosome initiation and/or recruitment, as determined by the polysome profiling methods disclosed herein and/or known in the field.

Provided herein are methods of identifying a TIE capable of initiating, promoting, and/or driving translation comprising the steps of: (a) obtaining a pool of RNA molecules comprising barcode sequence and TIE, (b) transfecting the RNA molecules into a cell comprising one or more ribosomes, (c) lysing the cell and adding the content of the lysed cell into a gradient to fractionate based on the ribosomal load, and (d) selecting the RNA and TIE combination having high ribosomal load. In some embodiments, the RNA is circular RNA.

Provided herein are methods of identifying a TIE having high translation efficiency comprising the steps of: (a) obtaining a pool of RNA molecules comprising barcode sequence and TIE, (b) transfecting the RNA molecules into a cell comprising one or more ribosomes, (c) lysing the cell and adding the content of the lysed cell into a gradient to fractionate based on the ribosomal load, and (d) selecting the RNA and TIE combination having high ribosomal load. In some embodiments, the RNA is circular RNA.

### 6. Overview of Transfer Vehicle & Other Delivery Mechanisms

### A. Ionizable Lipids

In certain embodiments, disclosed herein are ionizable lipids that may be used as a component of a transfer vehicle to facilitate or enhance the delivery and release of circular RNA to one or more target cells (e.g., by permeating or fusing with the lipid membranes of such target cells). In certain embodiments, an ionizable lipid comprises one or more cleavable functional groups (e.g., a disulfide) that allow, for example, a hydrophilic functional head-group to dissociate from a lipophilic functional tail-group of the compound (e.g., upon exposure to oxidative, reducing or acidic conditions), thereby facilitating a phase transition in the lipid bilayer of the one or more target cells.

In some embodiments, an ionizable lipid is a lipid as described in international patent application PCT/US2018/058555.

In some embodiments, the transfer vehicle comprises Lipid A, Lipid B, Lipid C, and/or Lipid D. In some embodiments, inclusion of Lipid A, Lipid B, Lipid C, and/or Lipid D improves encapsulation and/or endosomal escape.

In some embodiments, an ionizable lipid is Lipid A, which is (9Z,12Z)-3-((4,4-bis(octyloxy)butanoyl)oxy)-2-((((3-(diethylamino)propoxy)carbonyl)oxy)methyl)propyl octadeca9,12-dienoate, also called 3-((4,44bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

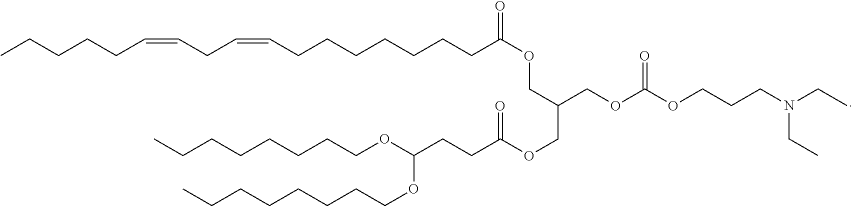 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments, an ionizable lipid of the disclosure is selected from Table 2.

TABLE 2
Exemplary Ionizable Lipids

| Lipid | Structure |
|---|---|
| 10a-26 | 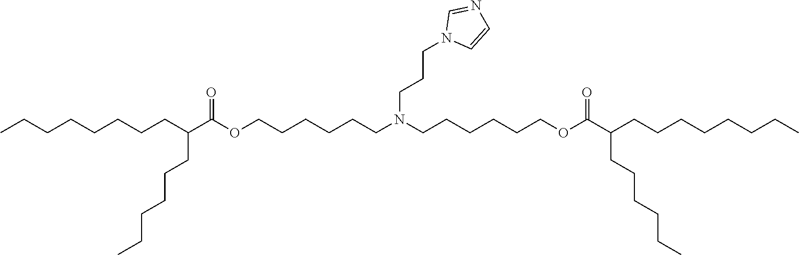 [SMILES: CCCCCCCCC(CCCCCC)C(=O)OCCCCCCN(CCCCCCOC(=O)C(CCCCCC)CCCCCCCC)CCCn1ccnc1] |
| 10a-27 | 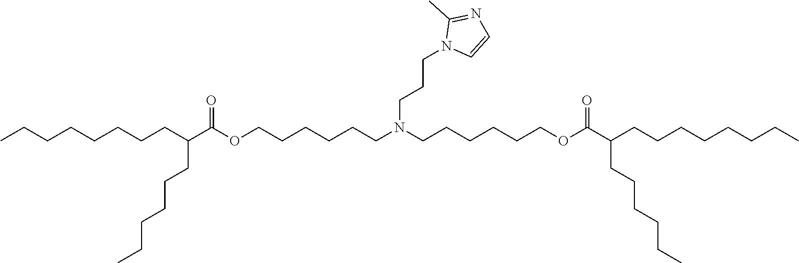 [SMILES: CCCCCCCCC(CCCCCC)C(=O)OCCCCCCN(CCCCCCOC(=O)C(CCCCCC)CCCCCCCC)CCCn1ccnc1C] |
| 10a-53 | 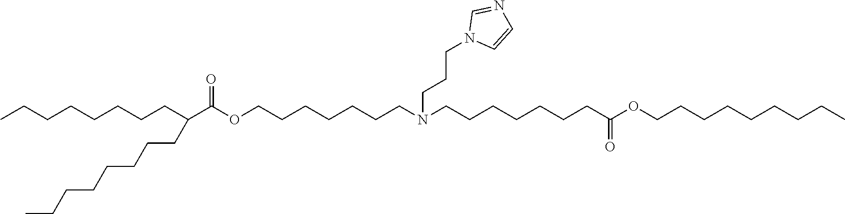 [SMILES: CCCCCCCCCOC(=O)CCCCCCCN(CCCCCCCOC(=O)C(CCCCCCCC)CCCCCCCC)CCCn1ccnc1] |
| 10a-54 | 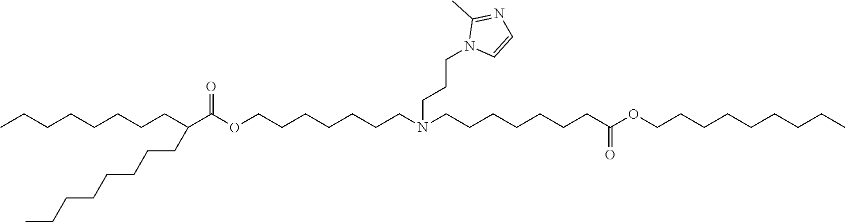 [SMILES: CCCCCCCCCOC(=O)CCCCCCCN(CCCCCCCOC(=O)C(CCCCCCCC)CCCCCCCC)CCCn1ccnc1C] |
| 10b-15 | 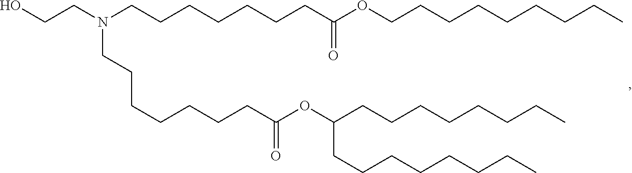 [SMILES: CCCCCCCCCOC(=O)CCCCCCCN(CCO)CCCCCCCC(=O)OC(CCCCCCCC)CCCCCCCC] |
| 22-S14 | 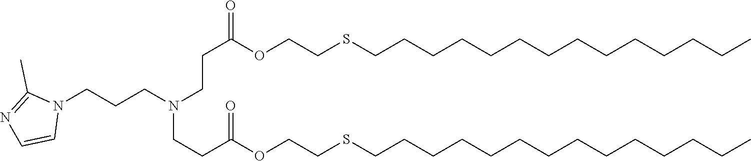 [SMILES: CCCCCCCCCCCCCCSCCOC(=O)CCN(CCCn1ccnc1C)CCC(=O)OCCSCCCCCCCCCCCCCC] |
| 93-S14 | 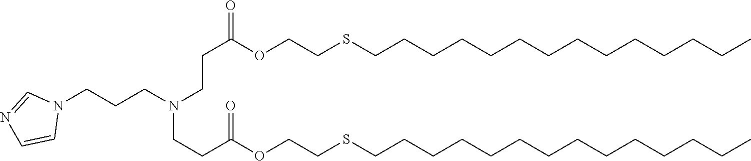 [SMILES: CCCCCCCCCCCCCCSCCOC(=O)CCN(CCCn1ccnc1)CCC(=O)OCCSCCCCCCCCCCCCCC] |
| Lipid 39 | 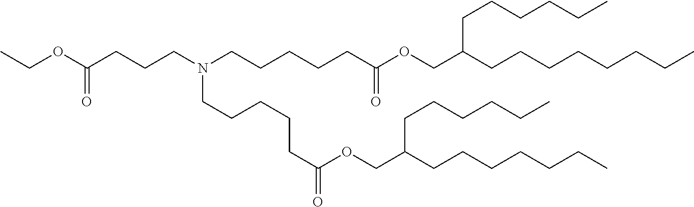 [SMILES: CCCCCCCCC(CCCCCC)COC(=O)CCCCCN(CCCCCC(=O)OCC(CCCCCC)CCCCCCC)CCCC(=O)OCC] |

Table of Additional Exemplary Ionizable Lipids (Table B)

| Lipid | Structure |
|---|---|
| 1 | 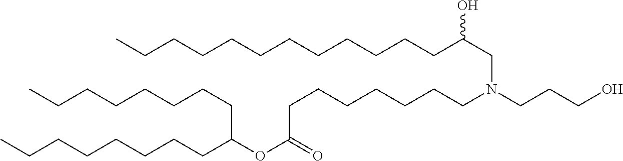 [SMILES: CCCCCCCCCCCCC(O)CN(CCCO)CCCCCCCC(=O)OC(CCCCCCCC)CCCCCCCC] |
| 2 | 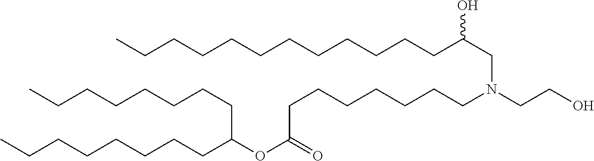 [SMILES: CCCCCCCCCCCCC(O)CN(CCO)CCCCCCCC(=O)OC(CCCCCCCC)CCCCCCCC] |
| 3 | 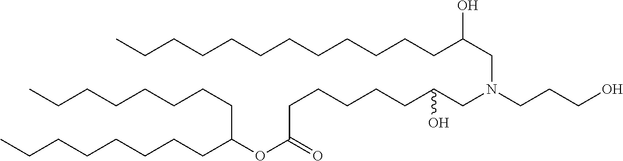 [SMILES: CCCCCCCCCCCCC(O)CN(CCCO)CC(O)CCCCCC(=O)OC(CCCCCCCC)CCCCCCCC] |
| 4 | 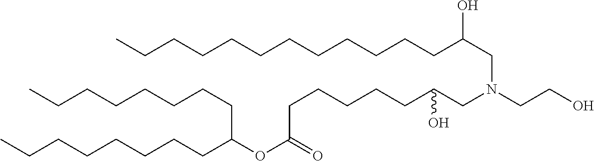 [SMILES: CCCCCCCCCCCCC(O)CN(CCO)CC(O)CCCCCC(=O)OC(CCCCCCCC)CCCCCCCC] |
| 5 | 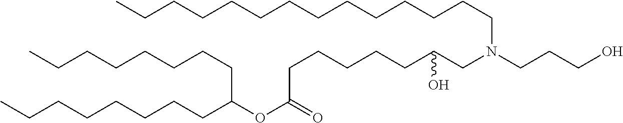 [SMILES: CCCCCCCCCCCCCCN(CCCO)CC(O)CCCCCC(=O)OC(CCCCCCCC)CCCCCCCC] |
| 6 | 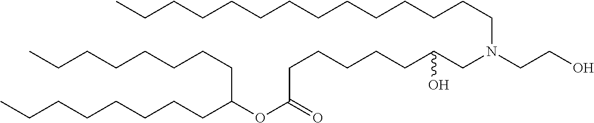 [SMILES: CCCCCCCCCCCCCCN(CCO)CC(O)CCCCCC(=O)OC(CCCCCCCC)CCCCCCCC] |
| 7 | 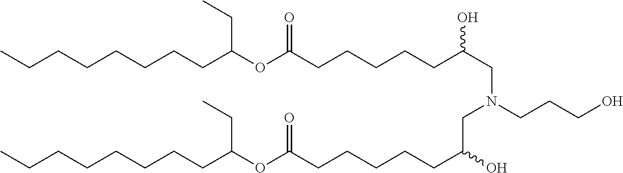 [SMILES: CCCCCCCCC(CC)OC(=O)CCCCCC(O)CN(CCCO)CC(O)CCCCCC(=O)OC(CC)CCCCCCCC] |
| 8 | 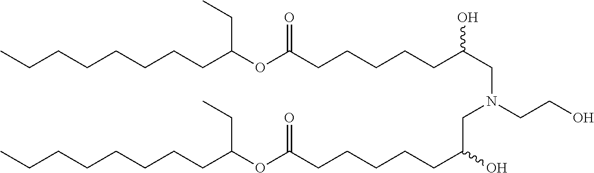 [SMILES: CCCCCCCCC(CC)OC(=O)CCCCCC(O)CN(CCO)CC(O)CCCCCC(=O)OC(CC)CCCCCCCC] |
| 9 | 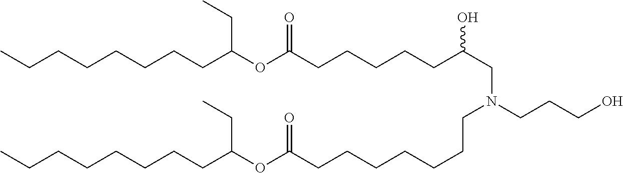 [SMILES: CCCCCCCCC(CC)OC(=O)CCCCCCCN(CCCO)CC(O)CCCCCC(=O)OC(CC)CCCCCCCC] |
| 10 | 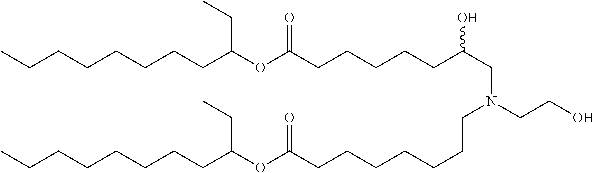 [SMILES: CCCCCCCCC(CC)OC(=O)CCCCCCCN(CCO)CC(O)CCCCCC(=O)OC(CC)CCCCCCCC] |
| 11 | 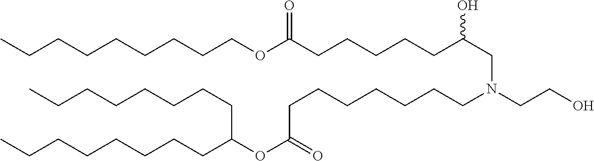 [SMILES: CCCCCCCCCOC(=O)CCCCCC(O)CN(CCO)CCCCCCCC(=O)OC(CCCCCCCC)CCCCCCCC] |
| 12 | 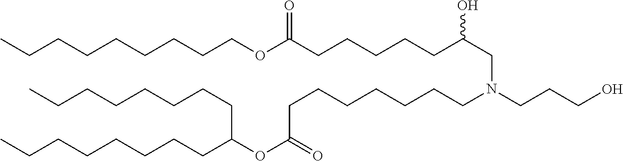 [SMILES: CCCCCCCCCOC(=O)CCCCCC(O)CN(CCCO)CCCCCCCC(=O)OC(CCCCCCCC)CCCCCCCC] |
| 13 | 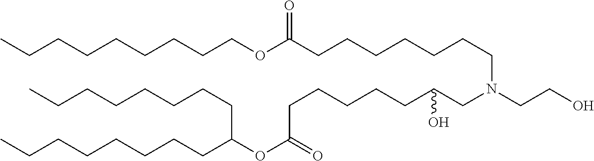 [SMILES: CCCCCCCCCOC(=O)CCCCCCCN(CCO)CC(O)CCCCCC(=O)OC(CCCCCCCC)CCCCCCCC] |
| 14 | 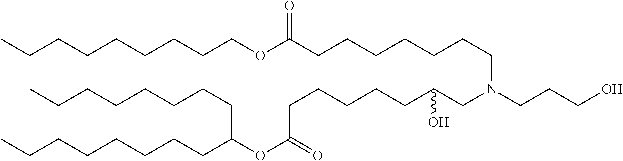 [SMILES: CCCCCCCCCOC(=O)CCCCCCCN(CCCO)CC(O)CCCCCC(=O)OC(CCCCCCCC)CCCCCCCC] |
| 15 | 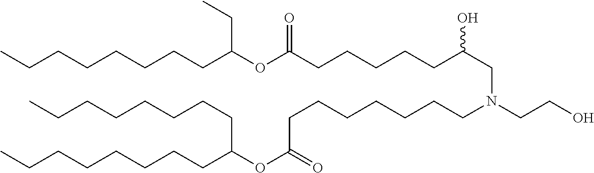 [SMILES: CCCCCCCCC(CC)OC(=O)CCCCCC(O)CN(CCO)CCCCCCCC(=O)OC(CCCCCCCC)CCCCCCCC] |
| 16 | 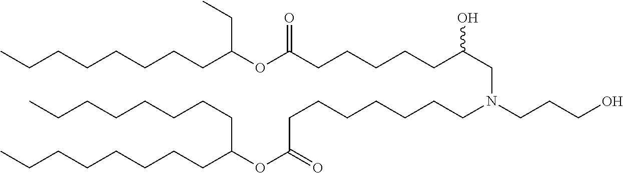 [SMILES: CCCCCCCCC(CC)OC(=O)CCCCCC(O)CN(CCCO)CCCCCCCC(=O)OC(CCCCCCCC)CCCCCCCC] |
| 17 | 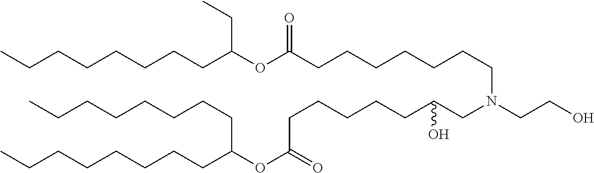 [SMILES: CCCCCCCCC(CC)OC(=O)CCCCCCCN(CCO)CC(O)CCCCCC(=O)OC(CCCCCCCC)CCCCCCCC] |
| 18 | 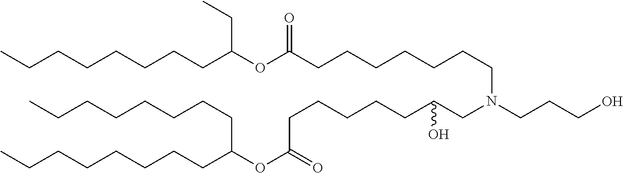 [SMILES: CCCCCCCCC(CC)OC(=O)CCCCCCCN(CCCO)CC(O)CCCCCC(=O)OC(CCCCCCCC)CCCCCCCC] |
| 19 | 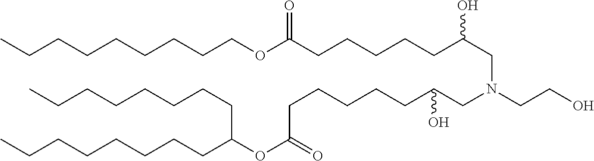 [SMILES: CCCCCCCCCOC(=O)CCCCCC(O)CN(CCO)CC(O)CCCCCC(=O)OC(CCCCCCCC)CCCCCCCC] |
| 20 | 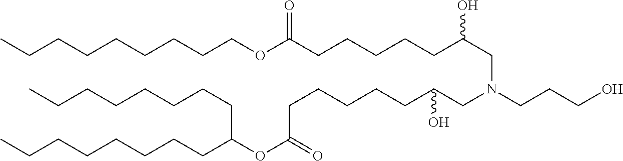 [SMILES: CCCCCCCCCOC(=O)CCCCCC(O)CN(CCCO)CC(O)CCCCCC(=O)OC(CCCCCCCC)CCCCCCCC] |
| 21 | 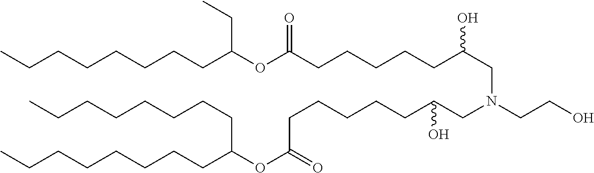 [SMILES: CCCCCCCCC(CC)OC(=O)CCCCCC(O)CN(CCO)CC(O)CCCCCC(=O)OC(CCCCCCCC)CCCCCCCC] |
| 22 | 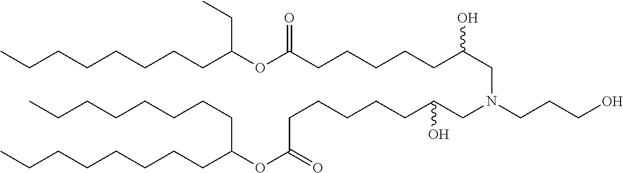 [SMILES: CCCCCCCCC(CC)OC(=O)CCCCCC(O)CN(CCCO)CC(O)CCCCCC(=O)OC(CCCCCCCC)CCCCCCCC] |
| 23 | 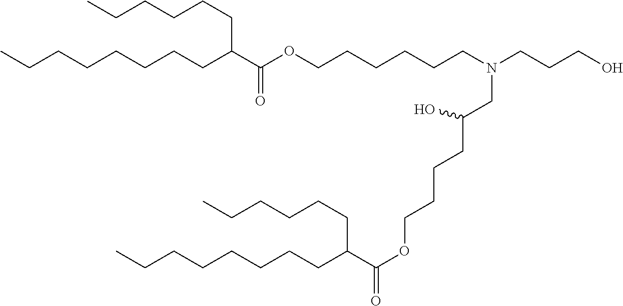 [SMILES: CCCCCCCCC(CCCCCC)C(=O)OCCCCCCN(CCCO)CC(O)CCCCOC(=O)C(CCCCCC)CCCCCCCC] |
| 24 | 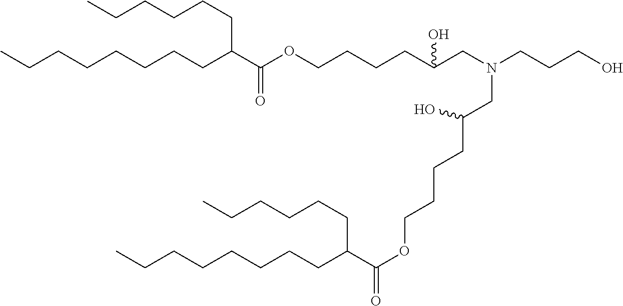 [SMILES: CCCCCCCCC(CCCCCC)C(=O)OCCCCC(O)CN(CCCO)CC(O)CCCCOC(=O)C(CCCCCC)CCCCCCCC] |
| 25 | 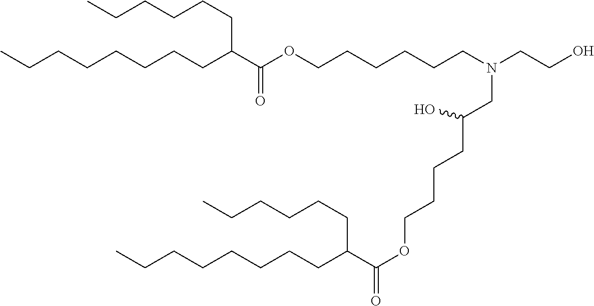 [SMILES: CCCCCCCCC(CCCCCC)C(=O)OCCCCCCN(CCO)CC(O)CCCCOC(=O)C(CCCCCC)CCCCCCCC] |
| 26 | 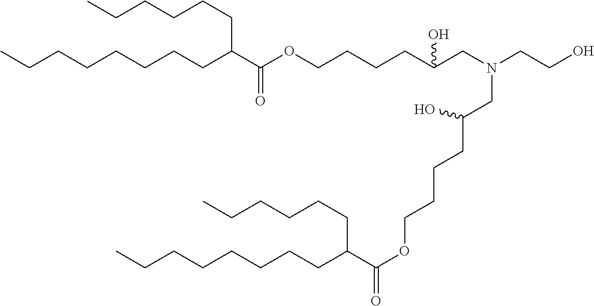 [SMILES: CCCCCCCCC(CCCCCC)C(=O)OCCCCC(O)CN(CCO)CC(O)CCCCOC(=O)C(CCCCCC)CCCCCCCC] |
| 27 | 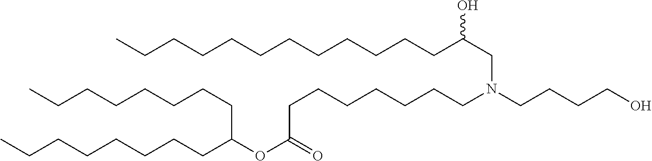 [SMILES: CCCCCCCCCCCCC(O)CN(CCCCO)CCCCCCCC(=O)OC(CCCCCCCC)CCCCCCCC] |
| 28 | 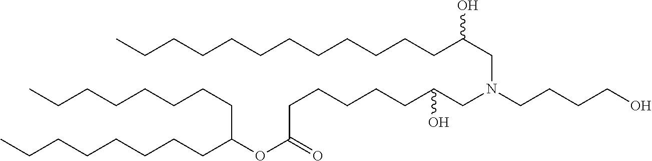 [SMILES: CCCCCCCCCCCCC(O)CN(CCCCO)CC(O)CCCCCC(=O)OC(CCCCCCCC)CCCCCCCC] |
| 29 | 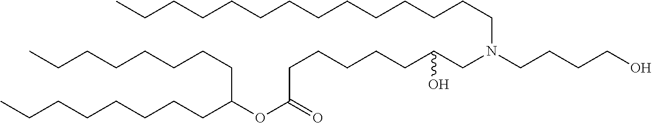 [SMILES: CCCCCCCCCCCCCCN(CCCCO)CC(O)CCCCCC(=O)OC(CCCCCCCC)CCCCCCCC] |
| 30 | 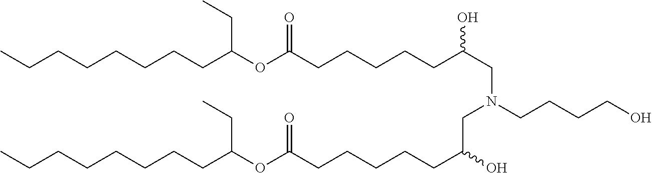 [SMILES: CCCCCCCCC(CC)OC(=O)CCCCCC(O)CN(CCCCO)CC(O)CCCCCC(=O)OC(CC)CCCCCCCC] |
| 31 | 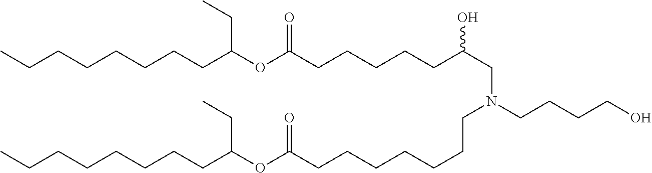 [SMILES: CCCCCCCCC(CC)OC(=O)CCCCCCCN(CCCCO)CC(O)CCCCCC(=O)OC(CC)CCCCCCCC] |
| 32 | 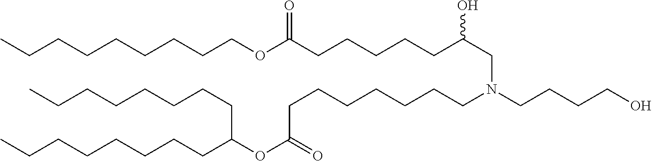 [SMILES: CCCCCCCCCOC(=O)CCCCCC(O)CN(CCCCO)CCCCCCCC(=O)OC(CCCCCCCC)CCCCCCCC] |
| 33 | 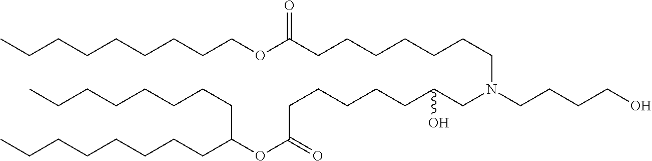 [SMILES: CCCCCCCCCOC(=O)CCCCCCCN(CCCCO)CC(O)CCCCCC(=O)OC(CCCCCCCC)CCCCCCCC] |
| 34 | 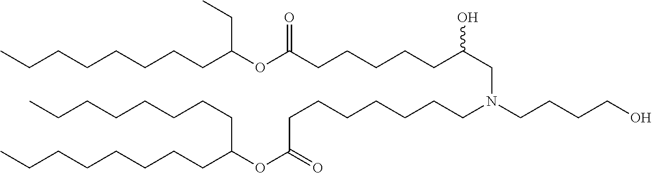 [SMILES: CCCCCCCCC(CC)OC(=O)CCCCCC(O)CN(CCCCO)CCCCCCCC(=O)OC(CCCCCCCC)CCCCCCCC] |
| 35 | 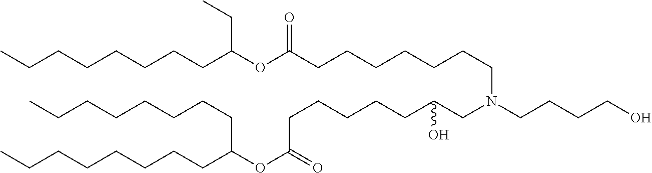 [SMILES: CCCCCCCCC(CC)OC(=O)CCCCCCCN(CCCCO)CC(O)CCCCCC(=O)OC(CCCCCCCC)CCCCCCCC] |
| 36 | 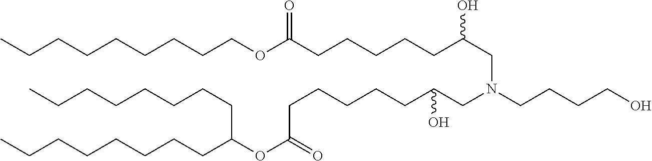 [SMILES: CCCCCCCCCOC(=O)CCCCCC(O)CN(CCCCO)CC(O)CCCCCC(=O)OC(CCCCCCCC)CCCCCCCC] |
| 37 | 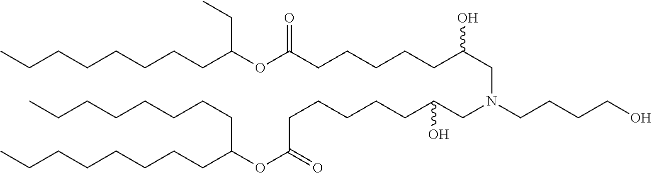 [SMILES: CCCCCCCCC(CC)OC(=O)CCCCCC(O)CN(CCCCO)CC(O)CCCCCC(=O)OC(CCCCCCCC)CCCCCCCC] |
| 38 | 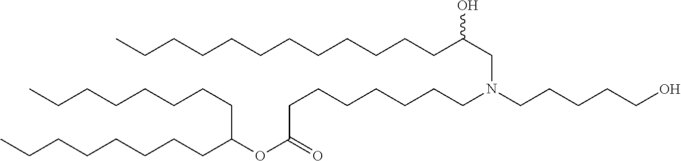 [SMILES: CCCCCCCCCCCCC(O)CN(CCCCCO)CCCCCCCC(=O)OC(CCCCCCCC)CCCCCCCC] |
| 39 | 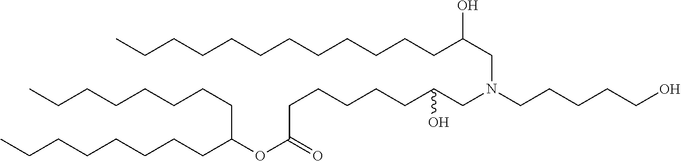 [SMILES: CCCCCCCCCCCCC(O)CN(CCCCCO)CC(O)CCCCCC(=O)OC(CCCCCCCC)CCCCCCCC] |
| 40 | 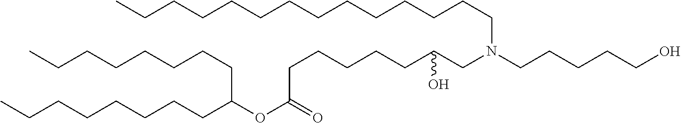 [SMILES: CCCCCCCCCCCCCCN(CCCCCO)CC(O)CCCCCC(=O)OC(CCCCCCCC)CCCCCCCC] |
| 41 | 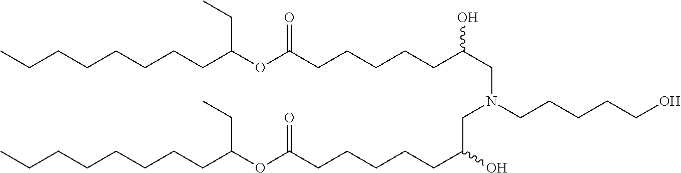 [SMILES: CCCCCCCCC(CC)OC(=O)CCCCCC(O)CN(CCCCCO)CC(O)CCCCCC(=O)OC(CC)CCCCCCCC] |
| 42 | 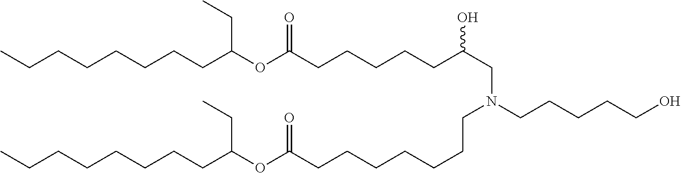 [SMILES: CCCCCCCCC(CC)OC(=O)CCCCCCCN(CCCCCO)CC(O)CCCCCC(=O)OC(CC)CCCCCCCC] |
| 43 | 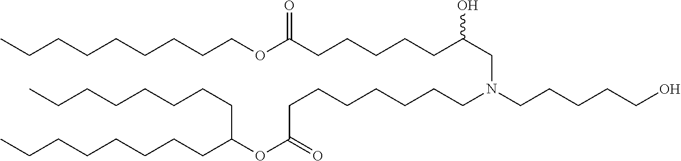 [SMILES: CCCCCCCCCOC(=O)CCCCCC(O)CN(CCCCCO)CCCCCCCC(=O)OC(CCCCCCCC)CCCCCCCC] |
| 44 | 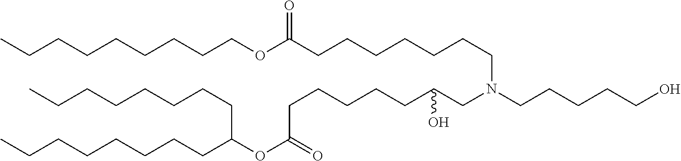 [SMILES: CCCCCCCCCOC(=O)CCCCCCCN(CCCCCO)CC(O)CCCCCC(=O)OC(CCCCCCCC)CCCCCCCC] |
| 45 | 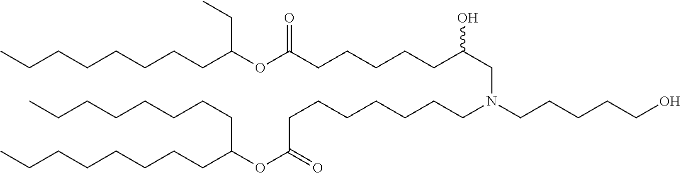 [SMILES: CCCCCCCCC(CC)OC(=O)CCCCCC(O)CN(CCCCCO)CCCCCCCC(=O)OC(CCCCCCCC)CCCCCCCC] |
| 46 | 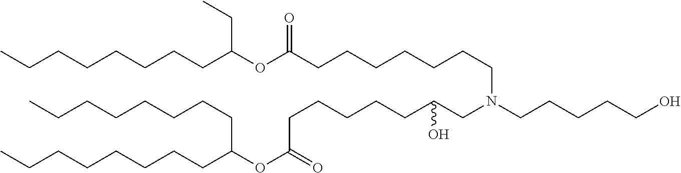 [SMILES: CCCCCCCCC(CC)OC(=O)CCCCCCCN(CCCCCO)CC(O)CCCCCC(=O)OC(CCCCCCCC)CCCCCCCC] |
| 47 | 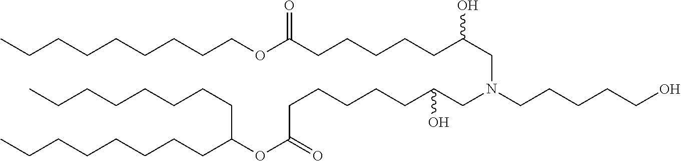 [SMILES: CCCCCCCCCOC(=O)CCCCCC(O)CN(CCCCCO)CC(O)CCCCCC(=O)OC(CCCCCCCC)CCCCCCCC] |
| 48 | 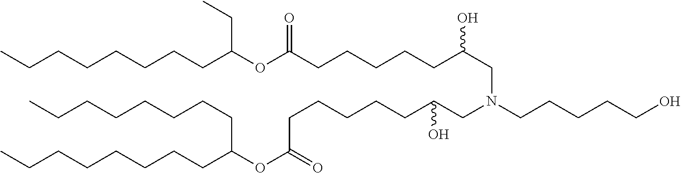 [SMILES: CCCCCCCCC(CC)OC(=O)CCCCCC(O)CN(CCCCCO)CC(O)CCCCCC(=O)OC(CCCCCCCC)CCCCCCCC] |
| 49 | 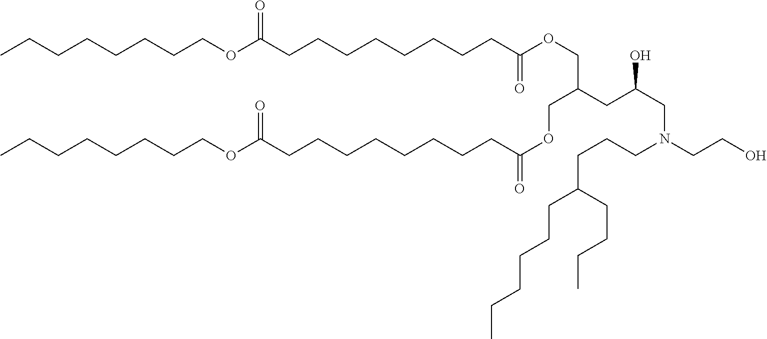 [SMILES: CCCCCCCCOC(=O)CCCCCCCCC(=O)OCC(COC(=O)CCCCCCCCC(=O)OCCCCCCCC)C[C@@H](O)CN(CCO)CCCC(CCCC)CCCCCC] |
| 50 | 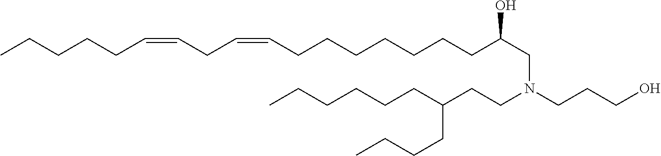 [SMILES: CCCCC/C=C\C/C=C\CCCCCCC[C@@H](O)CN(CCCO)CCC(CCCC)CCCCCC] |
| 51 | 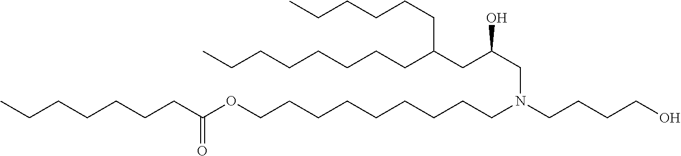 [SMILES: CCCCCCCCC(CCCCCC)C[C@@H](O)CN(CCCCO)CCCCCCCCCOC(=O)CCCCCCC] |
| 52 | 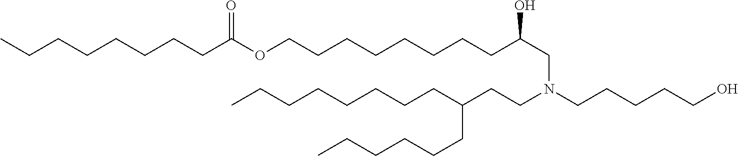 [SMILES: CCCCCCCCC(=O)OCCCCCCCC[C@@H](O)CN(CCCCCO)CCC(CCCCCC)CCCCCCCC] |
| 53 | 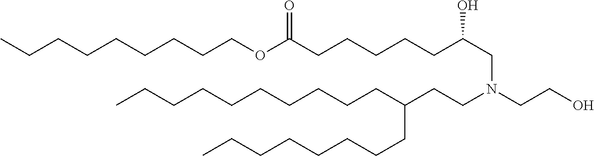 [SMILES: CCCCCCCCCCC(CCCCCCCC)CCN(CCO)C[C@@H](O)CCCCCC(=O)OCCCCCCCCC] |
| 54 | 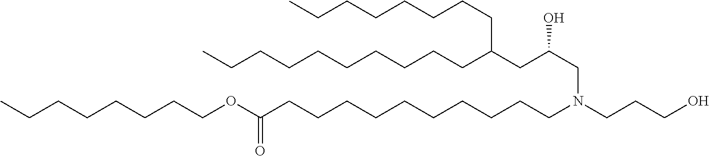 [SMILES: CCCCCCCCCCC(CCCCCCCC)C[C@H](O)CN(CCCO)CCCCCCCCCCC(=O)OCCCCCCCC] |
| 55 | 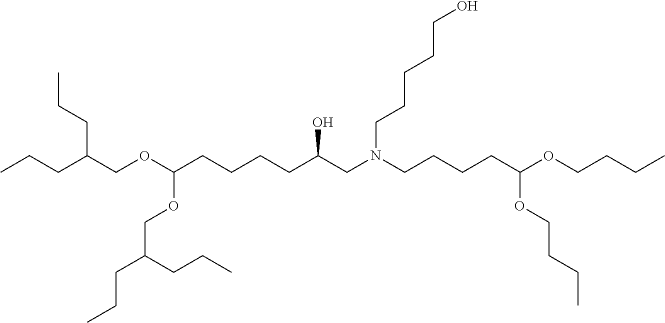 [SMILES: CCCCOC(CCCCN(CCCCCO)C[C@H](O)CCCCC(OCC(CCC)CCC)OCC(CCC)CCC)OCCCC] |
| 56 | 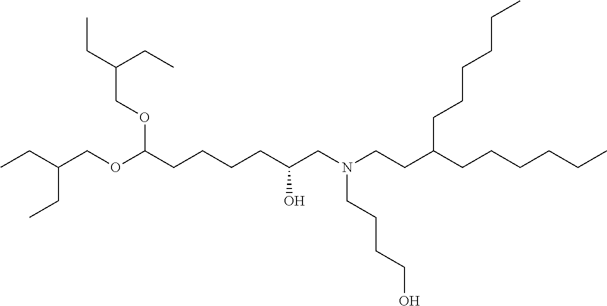 [SMILES: CCCCCCC(CCCCCC)CCN(CCCCO)C[C@H](O)CCCCC(OCC(CC)CC)OCC(CC)CC] |
| 57 | 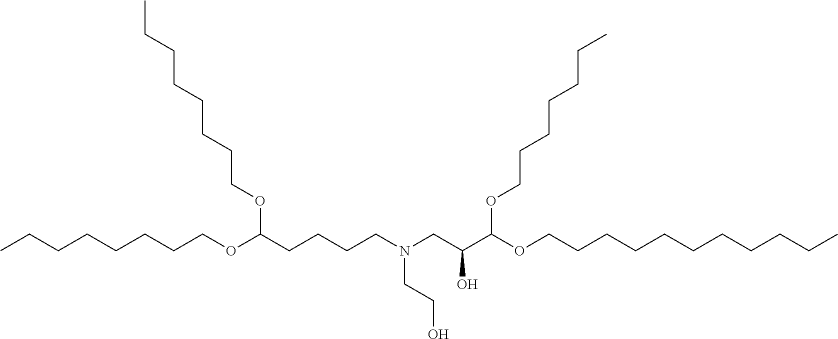 [SMILES: CCCCCCCCCCCOC(OCCCCCCC)[C@@H](O)CN(CCO)CCCCC(OCCCCCCCC)OCCCCCCCC] |
| 58 | 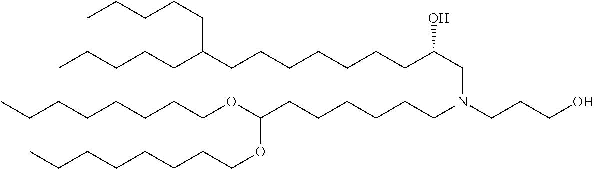 [SMILES: CCCCCCCCOC(CCCCCCN(CCCO)C[C@@H](O)CCCCCCCC(CCCCC)CCCCC)OCCCCCCCC] |
| 59 | 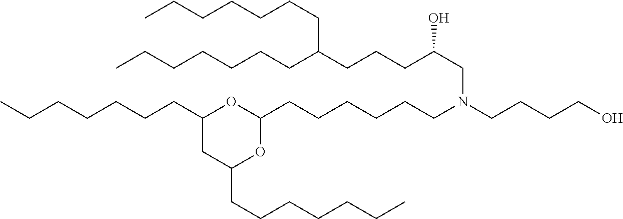 [SMILES: CCCCCCCC(CCCCCCC)CCC[C@H](O)CN(CCCCO)CCCCCCC1OC(CCCCCCC)CC(CCCCCCC)O1] |
| 60 | 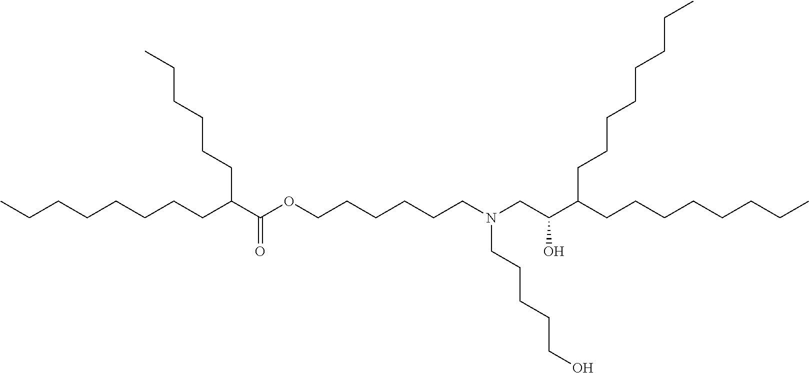 [SMILES: CCCCCCCCC(CCCCCC)C(=O)OCCCCCCN(CCCCCO)C[C@@H](O)C(CCCCCCCC)CCCCCCCC] |
| 61 | 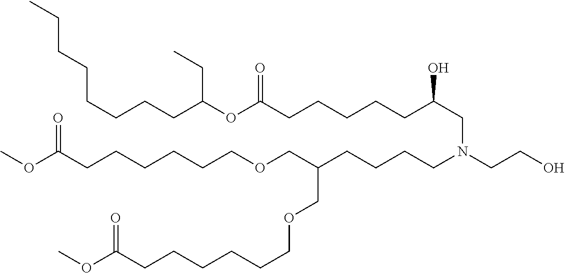 [SMILES: CCCCCCCCC(CC)OC(=O)CCCCC[C@@H](O)CN(CCO)CCCCC(COCCCCCCC(=O)OC)COCCCCCCC(=O)OC] |
| 62 | 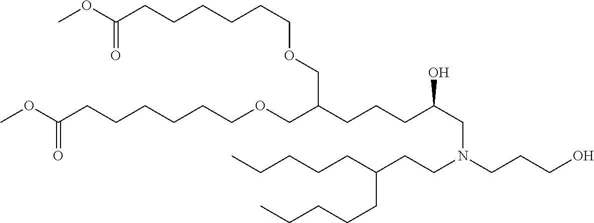 [SMILES: CCCCCC(CCCCC)CCN(CCCO)C[C@H](O)CCCC(COCCCCCCC(=O)OC)COCCCCCCC(=O)OC] |
| 63 | 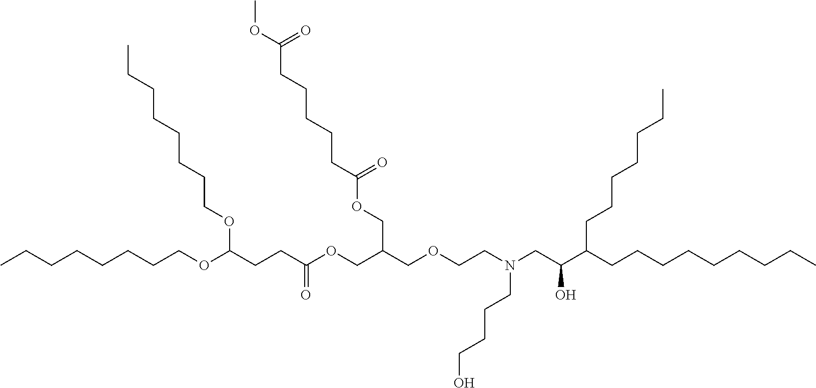 [SMILES: CCCCCCCCCC(CCCCCCC)[C@@H](O)CN(CCCCO)CCOCC(COC(=O)CCCCCC(=O)OC)COC(=O)CCC(OCCCCCCCC)OCCCCCCCC] |
| 64 | 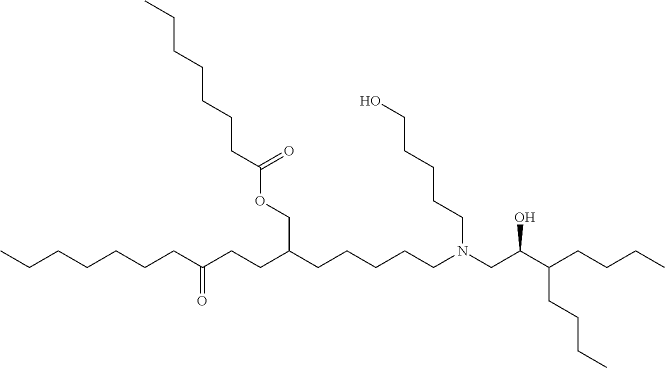 [SMILES: CCCCCCCC(=O)CCC(CCCCCN(CCCCCO)C[C@@H](O)C(CCCC)CCCC)COC(=O)CCCCCCC] |
| 65 | 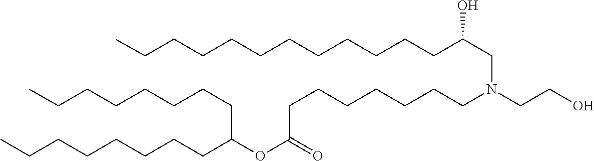 [SMILES: CCCCCCCCCCCC[C@H](O)CN(CCO)CCCCCCCC(=O)OC(CCCCCCCC)CCCCCCCC] |
| 66 | 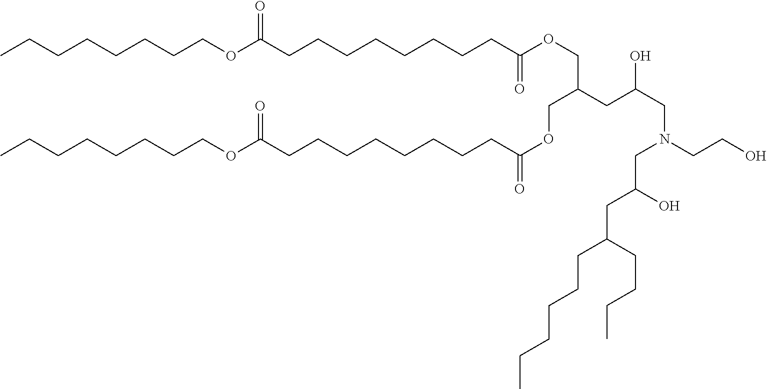 [SMILES: CCCCCCCCOC(=O)CCCCCCCCC(=O)OCC(COC(=O)CCCCCCCCC(=O)OCCCCCCCC)CC(O)CN(CCO)CC(O)CC(CCCC)CCCCCC] |
| 67 | 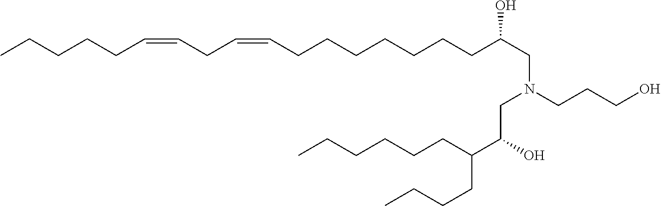 [SMILES: CCCCC/C=C\C/C=C\CCCCCCC[C@H](O)CN(CCCO)C[C@H](O)C(CCCC)CCCCCC] |
| 68 | 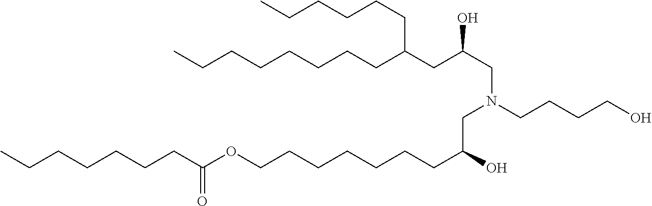 [SMILES: CCCCCCCCC(CCCCCC)C[C@@H](O)CN(CCCCO)C[C@@H](O)CCCCCCCOC(=O)CCCCCCC] |
| 69 | 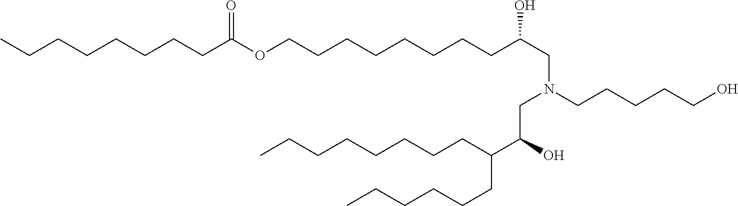 [SMILES: CCCCCCCCC(=O)OCCCCCCCC[C@H](O)CN(CCCCCO)C[C@@H](O)C(CCCCCC)CCCCCCCC] |
| 70 | 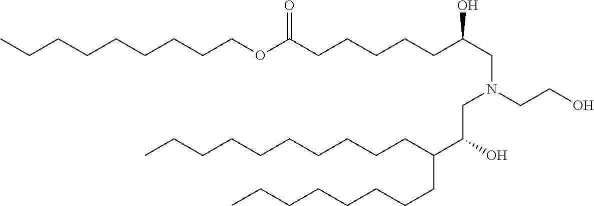 [SMILES: CCCCCCCCCCC(CCCCCCCC)[C@@H](O)CN(CCO)C[C@H](O)CCCCCC(=O)OCCCCCCCCC] |
| 71 | 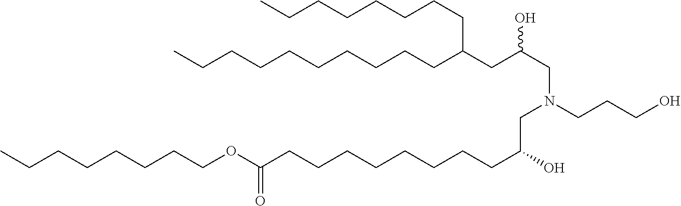 [SMILES: CCCCCCCCCCC(CCCCCCCC)CC(O)CN(CCCO)C[C@H](O)CCCCCCCCC(=O)OCCCCCCCC] |
| 72 | 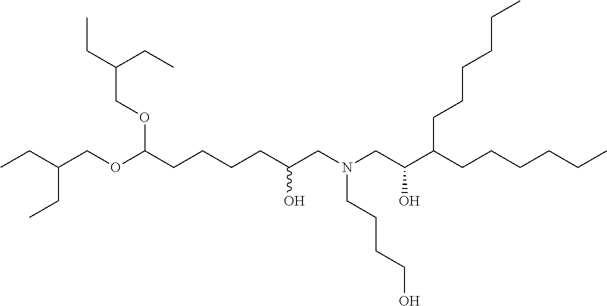 [SMILES: CCCCCCC(CCCCCC)[C@H](O)CN(CCCCO)CC(O)CCCCC(OCC(CC)CC)OCC(CC)CC] |
| 73 | 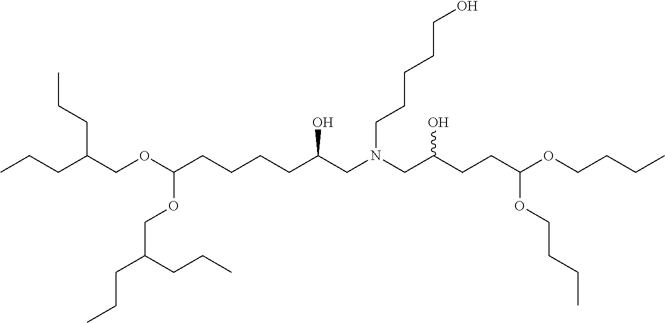 [SMILES: CCCCOC(CCC(O)CN(CCCCCO)C[C@H](O)CCCCC(OCC(CCC)CCC)OCC(CCC)CCC)OCCCC] |
| 74 | 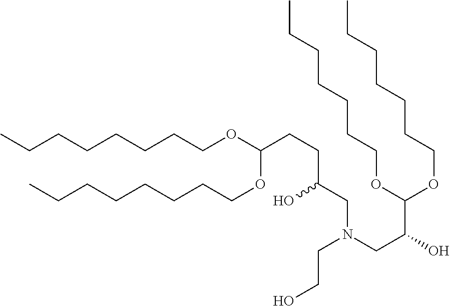 [SMILES: CCCCCCCCOC(CCC(O)CN(CCO)C[C@@H](O)C(OCCCCCCC)OCCCCCCC)OCCCCCCCC] |
| 75 | 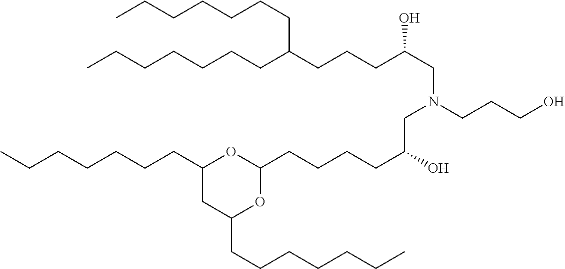 [SMILES: CCCCCCCC(CCCCCCC)CCC[C@H](O)CN(CCCO)C[C@H](O)CCCCC1OC(CCCCCCC)CC(CCCCCCC)O1] |
| 76 | 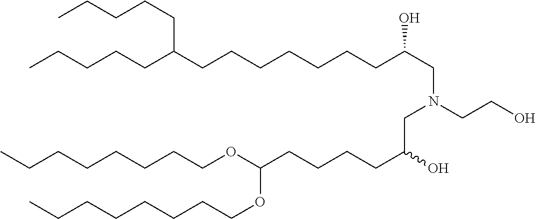 [SMILES: CCCCCCCCOC(CCCCC(O)CN(CCO)C[C@@H](O)CCCCCCCC(CCCCC)CCCCC)OCCCCCCCC] |
| 77 | 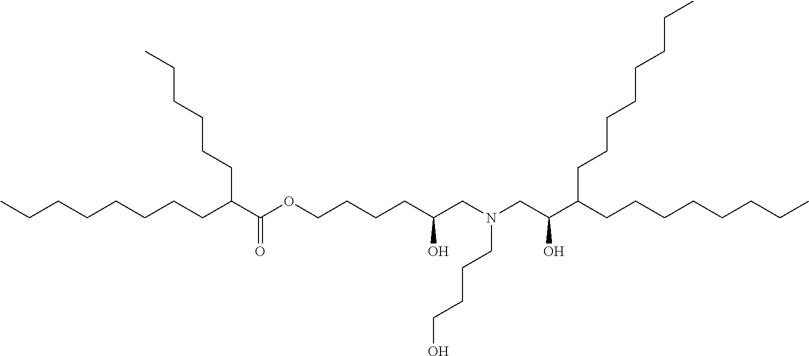 [SMILES: CCCCCCCCC(CCCCCC)C(=O)OCCCC[C@H](O)CN(CCCCO)C[C@H](O)C(CCCCCCCC)CCCCCCCC] |
| 78 | 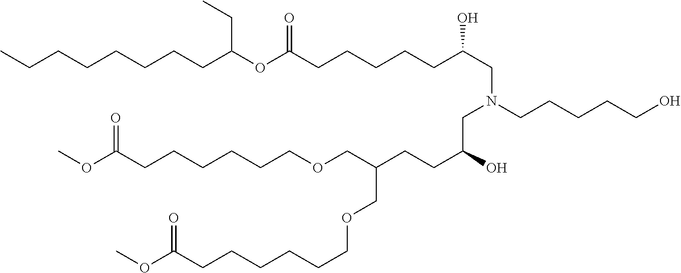 [SMILES: CCCCCCCCC(CC)OC(=O)CCCCC[C@H](O)CN(CCCCCO)C[C@@H](O)CCC(COCCCCCCC(=O)OC)COCCCCCCC(=O)OC] |
| 79 | 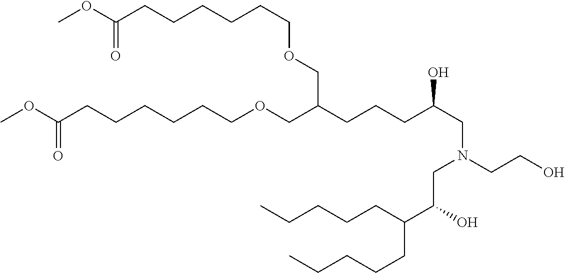 [SMILES: CCCCCC(CCCCC)[C@@H](O)CN(CCO)C[C@H](O)CCCC(COCCCCCCC(=O)OC)COCCCCCCC(=O)OC] |
| 80 | 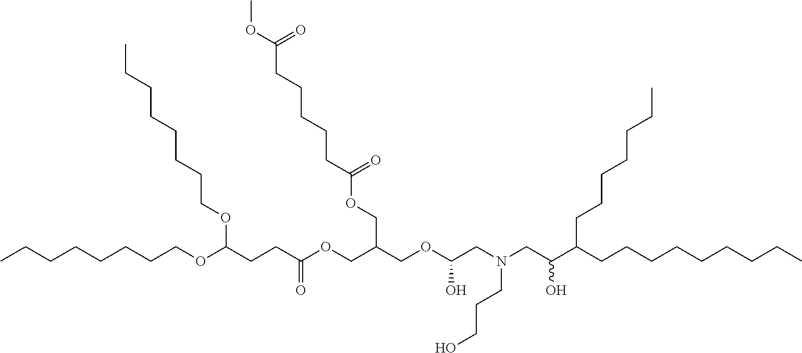 [SMILES: CCCCCCCCCC(CCCCCCC)C(O)CN(CCCO)C[C@H](O)OCC(COC(=O)CCCCCC(=O)OC)COC(=O)CCC(OCCCCCCCC)OCCCCCCCC] |
| 81 | 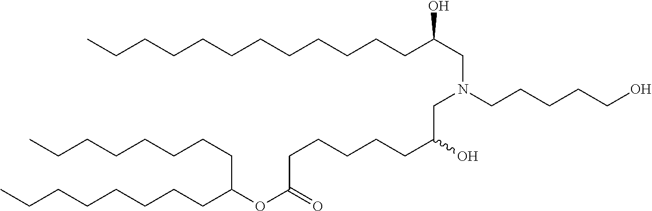 [SMILES: CCCCCCCCCCCC[C@@H](O)CN(CCCCCO)CC(O)CCCCCC(=O)OC(CCCCCCCC)CCCCCCCC] |
| 82 | 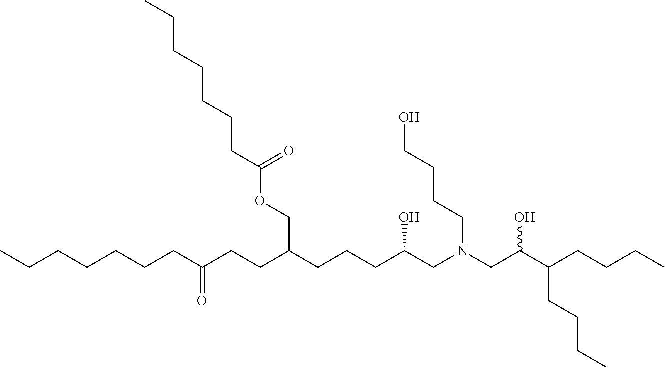 [SMILES: CCCCCCCC(=O)CCC(CCC[C@H](O)CN(CCCCO)CC(O)C(CCCC)CCCC)COC(=O)CCCCCCC] |
| 83 | 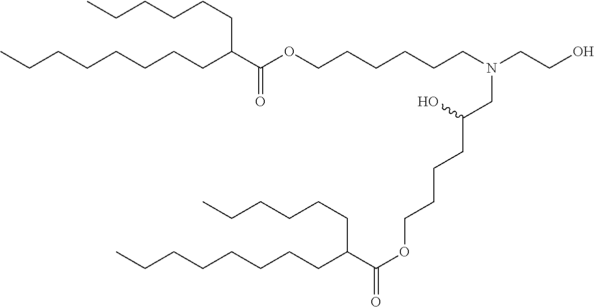 [SMILES: CCCCCCCCC(CCCCCC)C(=O)OCCCCCCN(CCO)CC(O)CCCCOC(=O)C(CCCCCC)CCCCCCCC] |
| 84 | 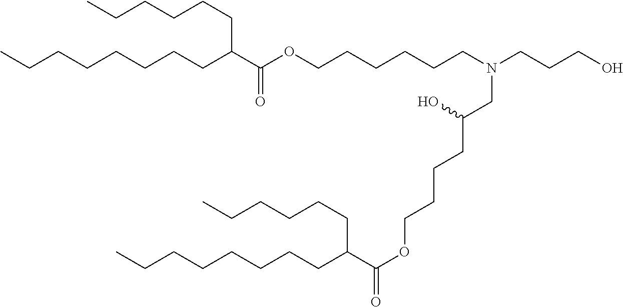 [SMILES: CCCCCCCCC(CCCCCC)C(=O)OCCCCCCN(CCCO)CC(O)CCCCOC(=O)C(CCCCCC)CCCCCCCC] |
| 85 | 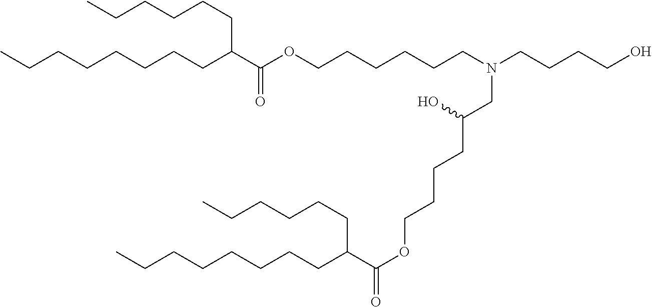 [SMILES: CCCCCCCCC(CCCCCC)C(=O)OCCCCCCN(CCCCO)CC(O)CCCCOC(=O)C(CCCCCC)CCCCCCCC] |
| 86 | 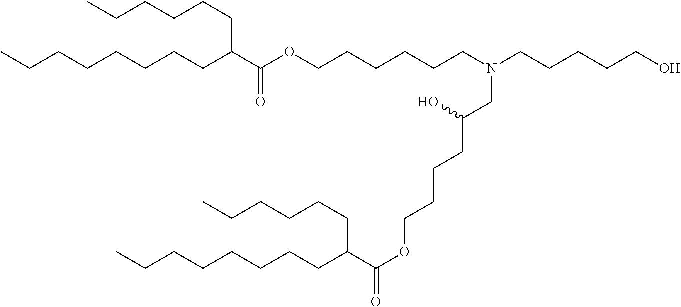 [SMILES: CCCCCCCCC(CCCCCC)C(=O)OCCCCCCN(CCCCCO)CC(O)CCCCOC(=O)C(CCCCCC)CCCCCCCC] |
| 87 | 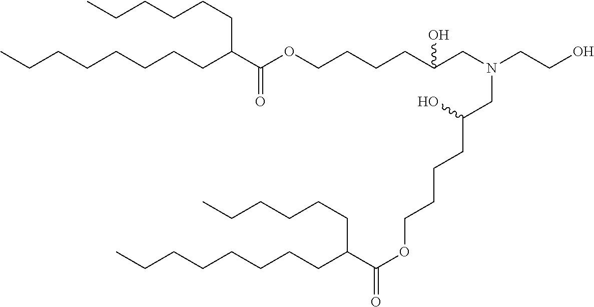 [SMILES: CCCCCCCCC(CCCCCC)C(=O)OCCCCC(O)CN(CCO)CC(O)CCCCOC(=O)C(CCCCCC)CCCCCCCC] |
| 88 | 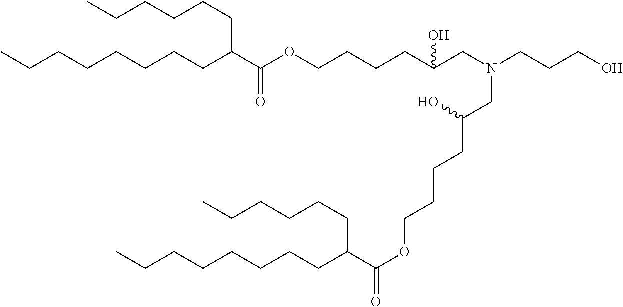 [SMILES: CCCCCCCCC(CCCCCC)C(=O)OCCCCC(O)CN(CCCO)CC(O)CCCCOC(=O)C(CCCCCC)CCCCCCCC] |
| 89 | 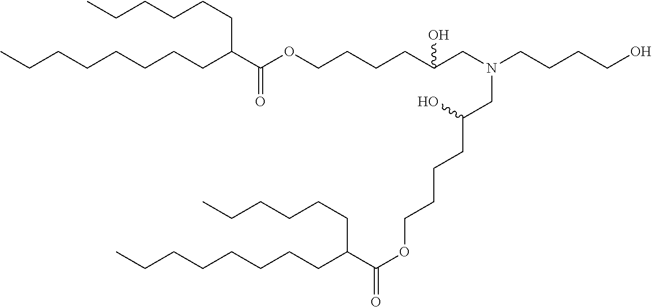 [SMILES: CCCCCCCCC(CCCCCC)C(=O)OCCCCC(O)CN(CCCCO)CC(O)CCCCOC(=O)C(CCCCCC)CCCCCCCC] |
| 90 | 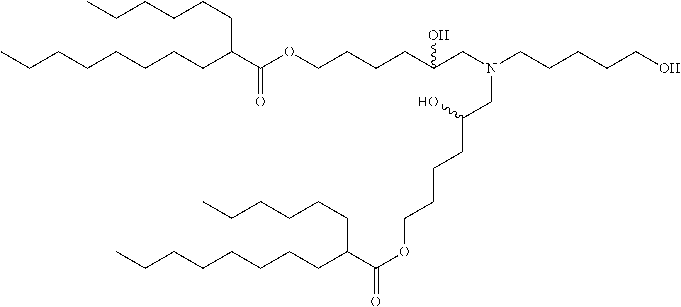 [SMILES: CCCCCCCCC(CCCCCC)C(=O)OCCCCC(O)CN(CCCCCO)CC(O)CCCCOC(=O)C(CCCCCC)CCCCCCCC] |
| 91 | 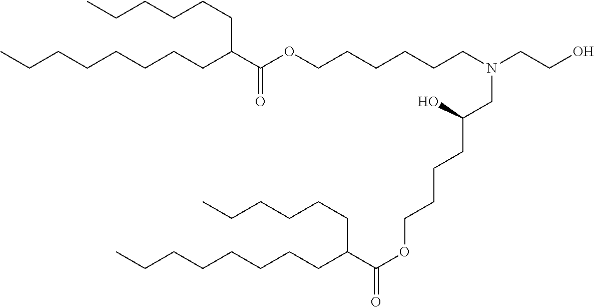 [SMILES: CCCCCCCCC(CCCCCC)C(=O)OCCCCCCN(CCO)C[C@H](O)CCCCOC(=O)C(CCCCCC)CCCCCCCC] |
| 92 | 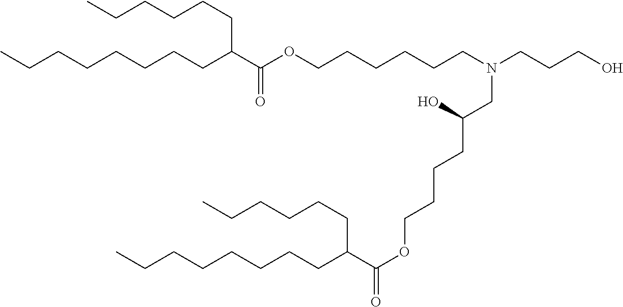 [SMILES: CCCCCCCCC(CCCCCC)C(=O)OCCCCCCN(CCCO)C[C@H](O)CCCCOC(=O)C(CCCCCC)CCCCCCCC] |
| 93 | 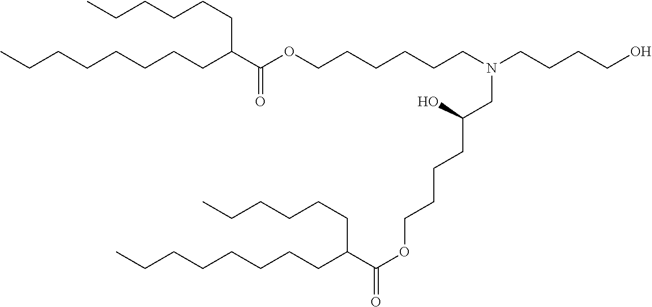 [SMILES: CCCCCCCCC(CCCCCC)C(=O)OCCCCCCN(CCCCO)C[C@H](O)CCCCOC(=O)C(CCCCCC)CCCCCCCC] |
| 94 | 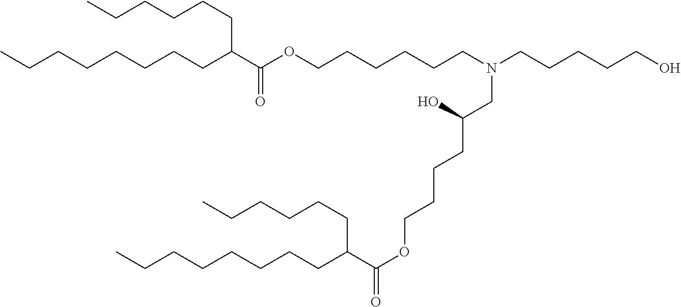 [SMILES: CCCCCCCCC(CCCCCC)C(=O)OCCCCCCN(CCCCCO)C[C@H](O)CCCCOC(=O)C(CCCCCC)CCCCCCCC] |
| 95 | 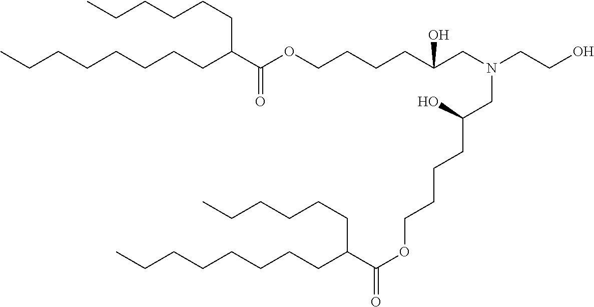 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 96 | 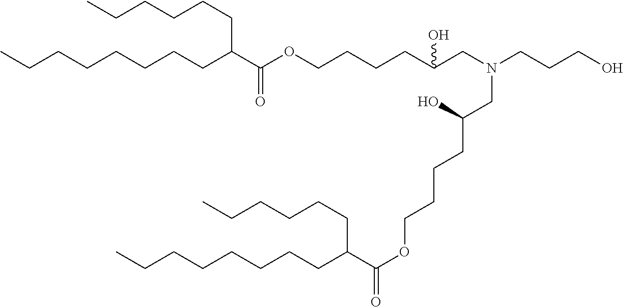 [SMILES: CCCCCCCCC(CCCCCC)C(=O)OCCCCC(O)CN(CCCO)C[C@H](O)CCCCOC(=O)C(CCCCCC)CCCCCCCC] |
| 97 | 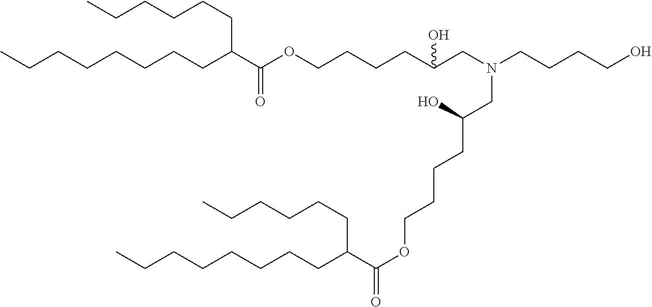 [SMILES: CCCCCCCCC(CCCCCC)C(=O)OCCCCC(O)CN(CCCCO)C[C@H](O)CCCCOC(=O)C(CCCCCC)CCCCCCCC] |
| 98 | 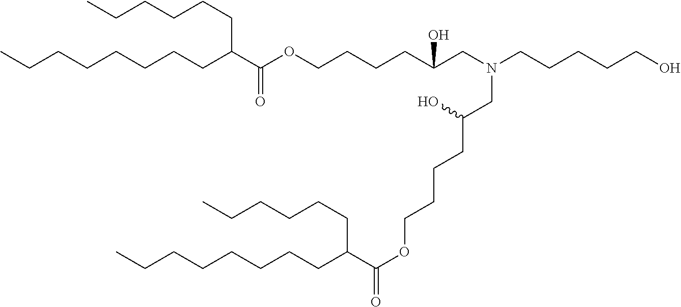 [SMILES: CCCCCCCCC(CCCCCC)C(=O)OCCCCC(O)CN(CCCCCO)C[C@@H](O)CCCCOC(=O)C(CCCCCC)CCCCCCCC] |
| 99 | 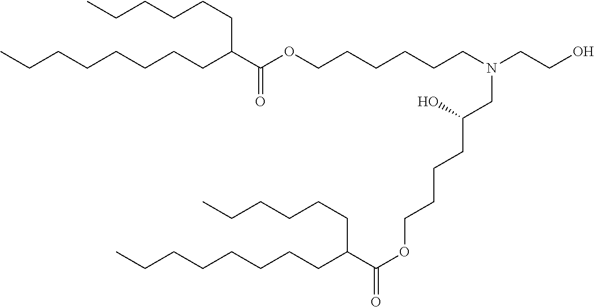 [SMILES: CCCCCCCCC(CCCCCC)C(=O)OCCCCCCN(CCO)C[C@@H](O)CCCCOC(=O)C(CCCCCC)CCCCCCCC] |
| 100 | 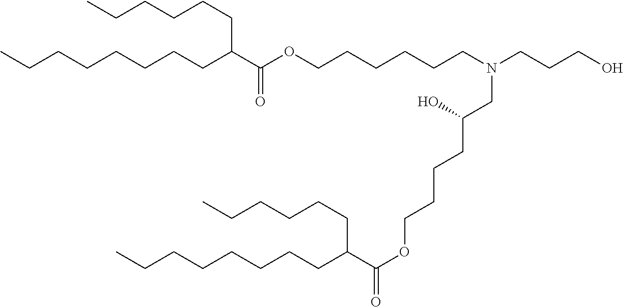 [SMILES: CCCCCCCCC(CCCCCC)C(=O)OCCCCCCN(CCCO)C[C@@H](O)CCCCOC(=O)C(CCCCCC)CCCCCCCC] |
| 101 | 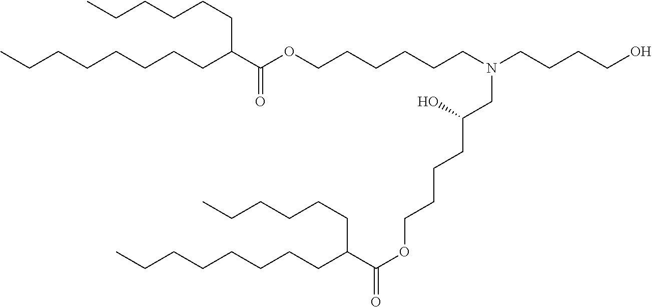 [SMILES: CCCCCCCCC(CCCCCC)C(=O)OCCCCCCN(CCCCO)C[C@@H](O)CCCCOC(=O)C(CCCCCC)CCCCCCCC] |
| 102 | 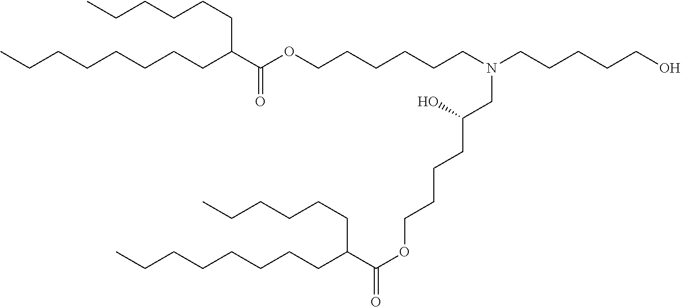 [SMILES: CCCCCCCCC(CCCCCC)C(=O)OCCCCCCN(CCCCCO)C[C@@H](O)CCCCOC(=O)C(CCCCCC)CCCCCCCC] |
| 103 | 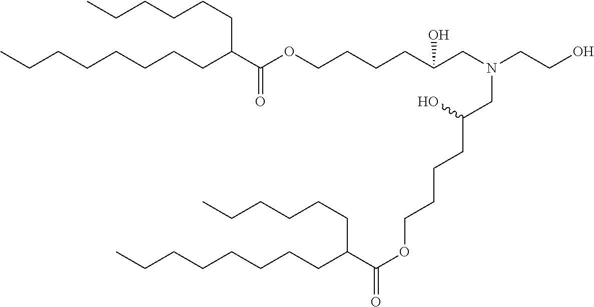 [SMILES: CCCCCCCCC(CCCCCC)C(=O)OCCCCC(O)CN(CCO)C[C@H](O)CCCCOC(=O)C(CCCCCC)CCCCCCCC] |
| 104 | 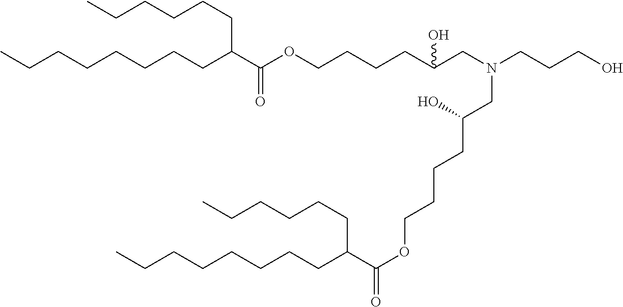 [SMILES: CCCCCCCCC(CCCCCC)C(=O)OCCCCC(O)CN(CCCO)C[C@@H](O)CCCCOC(=O)C(CCCCCC)CCCCCCCC] |
| 105 | 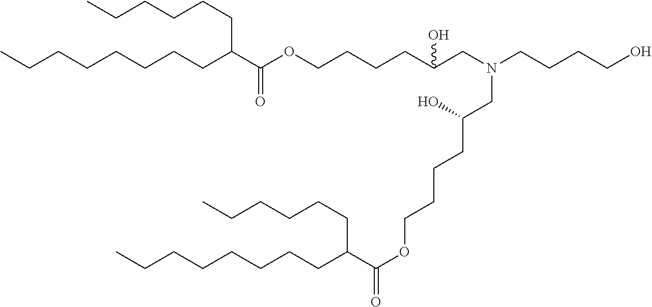 [SMILES: CCCCCCCCC(CCCCCC)C(=O)OCCCCC(O)CN(CCCCO)C[C@@H](O)CCCCOC(=O)C(CCCCCC)CCCCCCCC] |
| 106 | 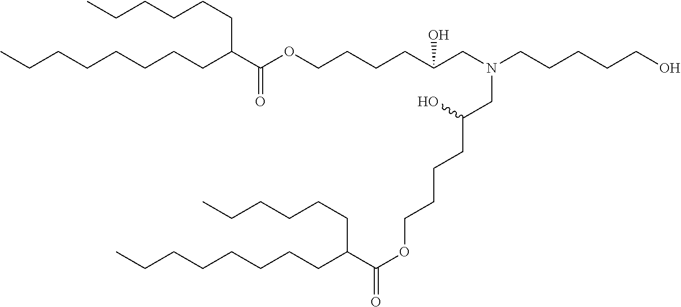 [SMILES: CCCCCCCCC(CCCCCC)C(=O)OCCCCC(O)CN(CCCCCO)C[C@H](O)CCCCOC(=O)C(CCCCCC)CCCCCCCC] |
| 107 | 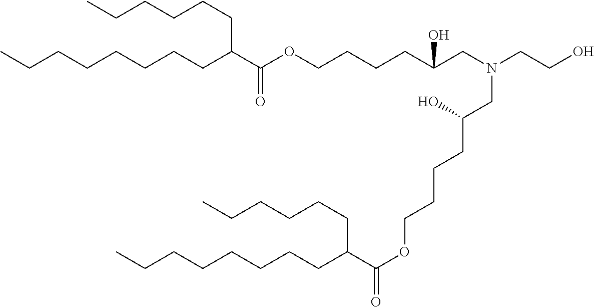 [SMILES: CCCCCCCCC(CCCCCC)C(=O)OCCCC[C@H](O)CN(CCO)C[C@@H](O)CCCCOC(=O)C(CCCCCC)CCCCCCCC] |
| 108 | 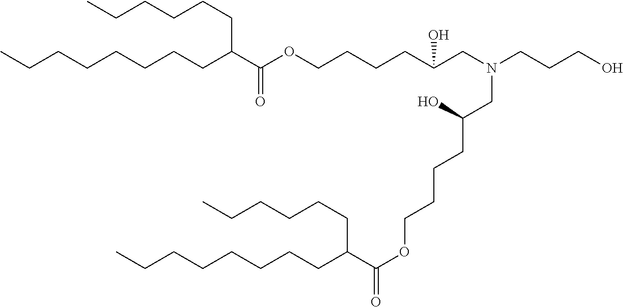 [SMILES: CCCCCCCCC(CCCCCC)C(=O)OCCCC[C@@H](O)CN(CCCO)C[C@H](O)CCCCOC(=O)C(CCCCCC)CCCCCCCC] |
| 109 | 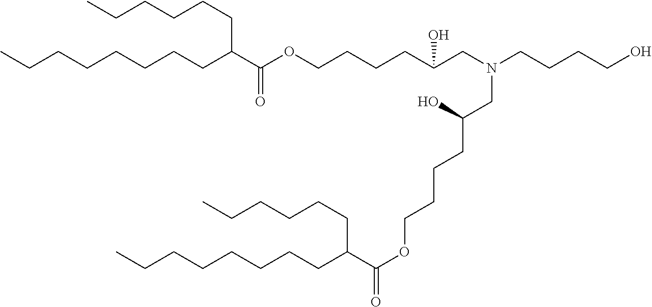 [SMILES: CCCCCCCCC(CCCCCC)C(=O)OCCCC[C@@H](O)CN(CCCCO)C[C@H](O)CCCCOC(=O)C(CCCCCC)CCCCCCCC] |
| 110 | 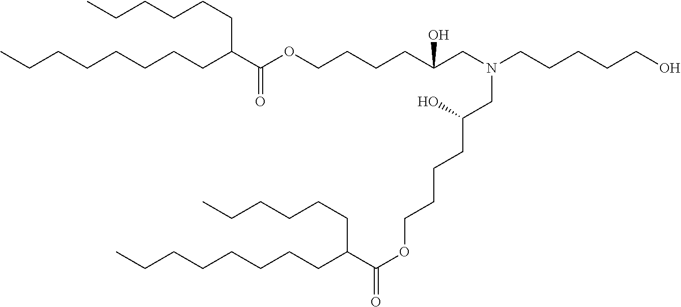 [SMILES: CCCCCCCCC(CCCCCC)C(=O)OCCCC[C@H](O)CN(CCCCCO)C[C@@H](O)CCCCOC(=O)C(CCCCCC)CCCCCCCC] |
| 111 | 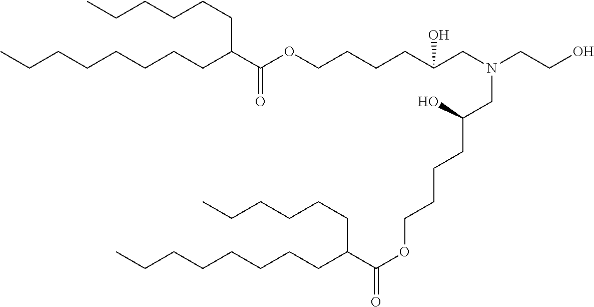 [SMILES: CCCCCCCCC(CCCCCC)C(=O)OCCCC[C@@H](O)CN(CCO)C[C@H](O)CCCCOC(=O)C(CCCCCC)CCCCCCCC] |
| 112 | 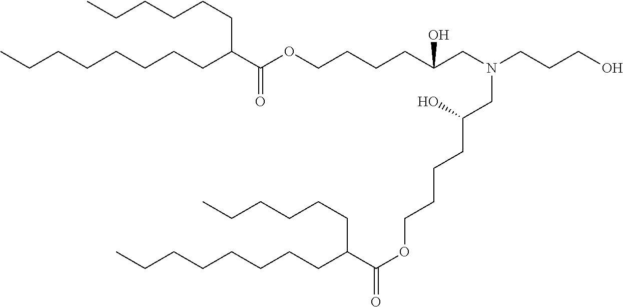 [SMILES: CCCCCCCCC(CCCCCC)C(=O)OCCCC[C@H](O)CN(CCCO)C[C@@H](O)CCCCOC(=O)C(CCCCCC)CCCCCCCC] |
| 113 | 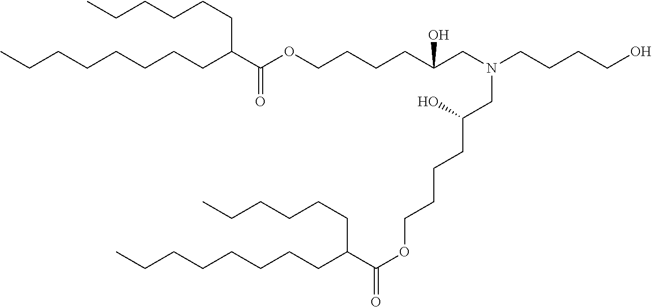 [SMILES: CCCCCCCCC(CCCCCC)C(=O)OCCCC[C@H](O)CN(CCCCO)C[C@@H](O)CCCCOC(=O)C(CCCCCC)CCCCCCCC] |
| 114 | 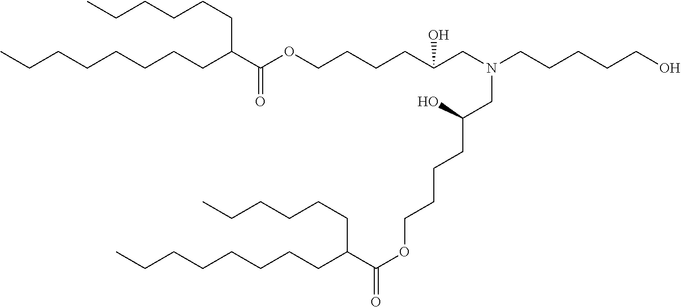 [SMILES: CCCCCCCCC(CCCCCC)C(=O)OCCCC[C@@H](O)CN(CCCCCO)C[C@H](O)CCCCOC(=O)C(CCCCCC)CCCCCCCC] |
| 115 | 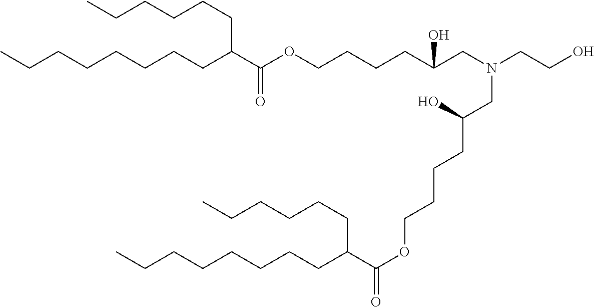 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 116 | 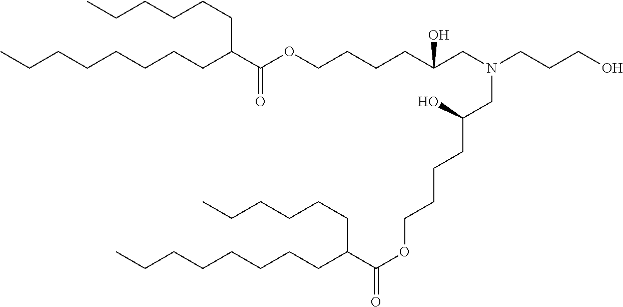 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 117 | 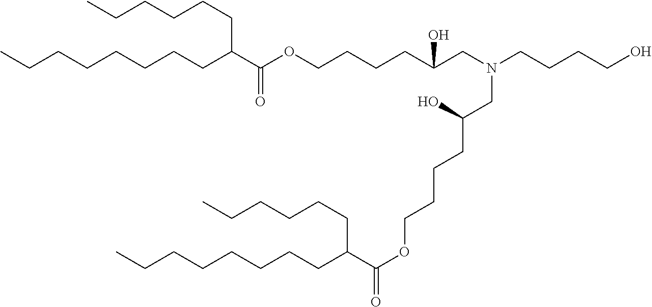 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 118 | 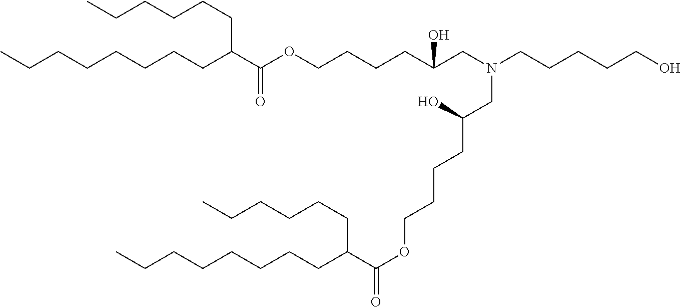 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 119 | 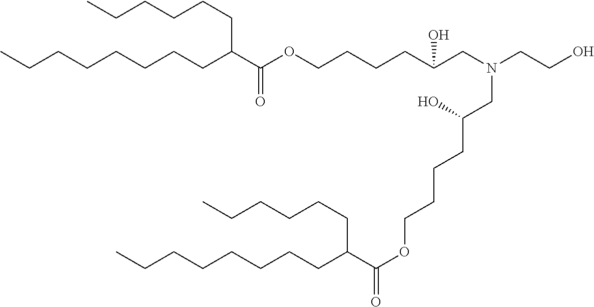 [SMILES: CCCCCCCCC(CCCCCC)C(=O)OCCCC[C@@H](O)CN(CCO)C[C@@H](O)CCCCOC(=O)C(CCCCCC)CCCCCCCC] |
| 120 | 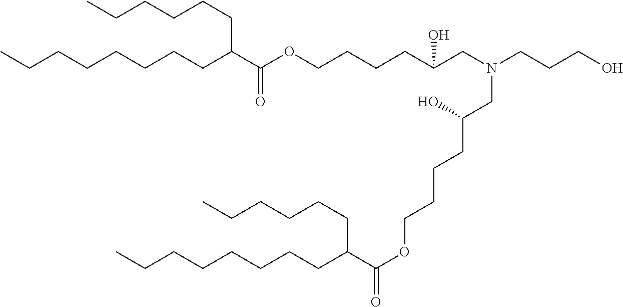 [SMILES: CCCCCCCCC(CCCCCC)C(=O)OCCCC[C@@H](O)CN(CCCO)C[C@@H](O)CCCCOC(=O)C(CCCCCC)CCCCCCCC] |
| 121 | 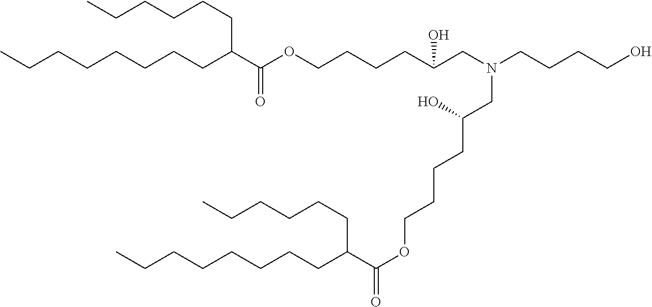 [SMILES: CCCCCCCCC(CCCCCC)C(=O)OCCCC[C@@H](O)CN(CCCCO)C[C@@H](O)CCCCOC(=O)C(CCCCCC)CCCCCCCC] |
| 122 | 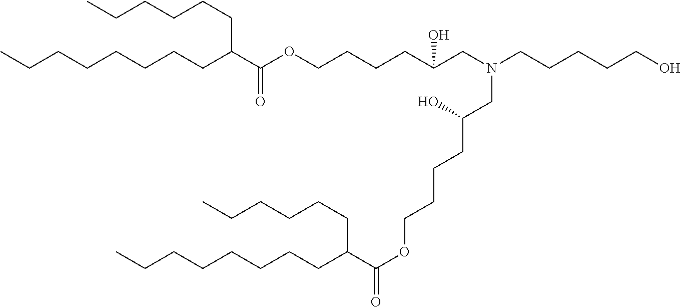 [SMILES: CCCCCCCCC(CCCCCC)C(=O)OCCCC[C@@H](O)CN(CCCCCO)C[C@@H](O)CCCCOC(=O)C(CCCCCC)CCCCCCCC] |
| 123 | 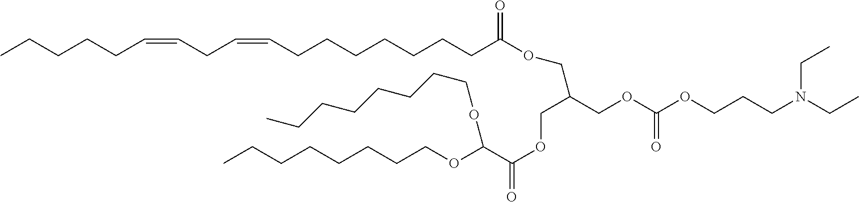 [SMILES: CCCCC/C=C\C/C=C\CCCCCCCC(=O)OCC(COC(=O)OCCCN(CC)CC)COC(=O)C(OCCCCCCCC)OCCCCCCCC] |
| 124 | 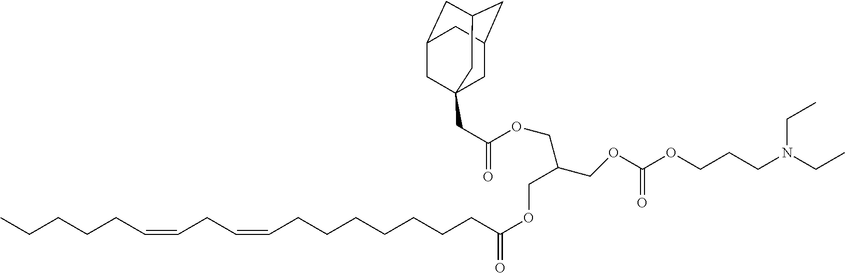 |
| 125 | 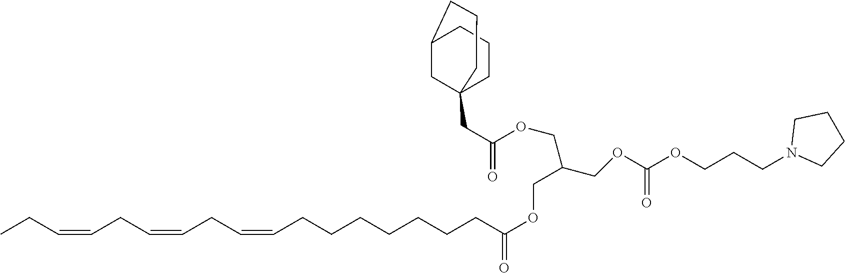 |
| 126 | 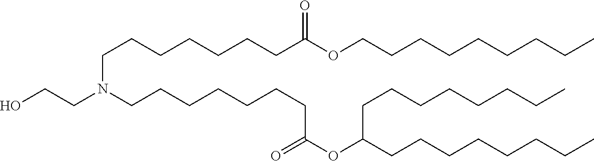 [SMILES: CCCCCCCCCOC(=O)CCCCCCCN(CCO)CCCCCCCC(=O)OC(CCCCCCCC)CCCCCCCC] |
| 127 | 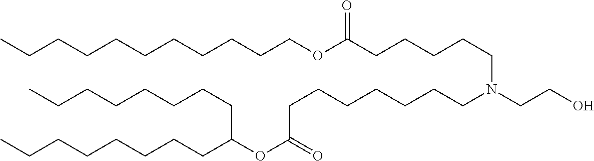 [SMILES: CCCCCCCCCCCOC(=O)CCCCCN(CCO)CCCCCCCC(=O)OC(CCCCCCCC)CCCCCCCC] |
| 128 | 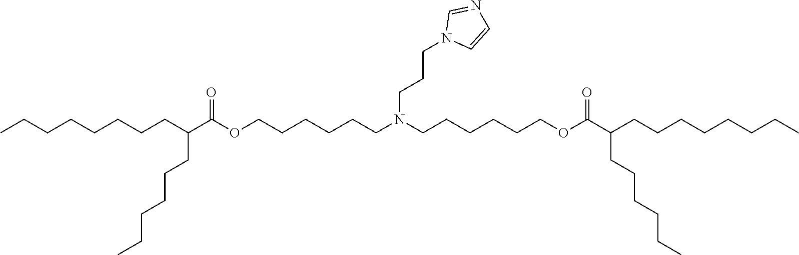 [SMILES: CCCCCCCCC(CCCCCC)C(=O)OCCCCCCN(CCCCCCOC(=O)C(CCCCCC)CCCCCCCC)CCCn1ccnc1] |
| 129 | 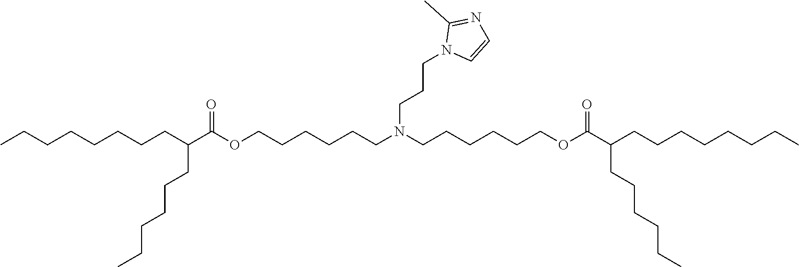 [SMILES: CCCCCCCCC(CCCCCC)C(=O)OCCCCCCN(CCCCCCOC(=O)C(CCCCCC)CCCCCCCC)CCCn1ccnc1C] |
| 130 | 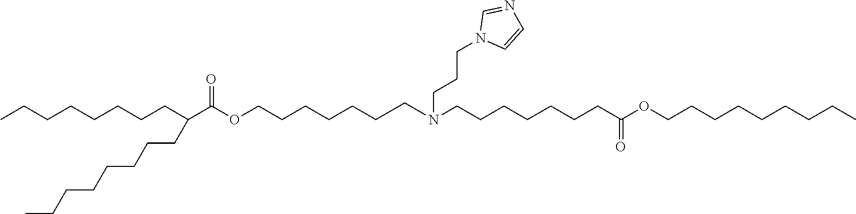 [SMILES: CCCCCCCCCOC(=O)CCCCCCCN(CCCCCCCOC(=O)C(CCCCCCCC)CCCCCCCC)CCCn1ccnc1] |
| 131 | 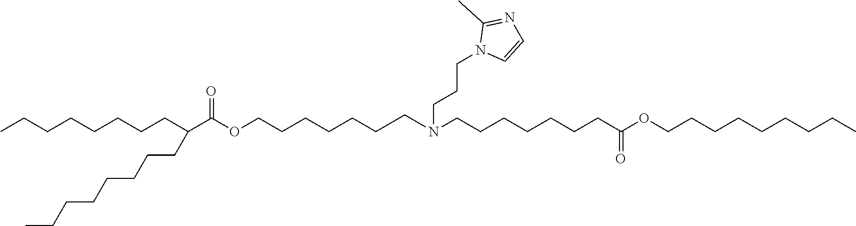 [SMILES: CCCCCCCCCOC(=O)CCCCCCCN(CCCCCCCOC(=O)C(CCCCCCCC)CCCCCCCC)CCCn1ccnc1C] |
| 132 | 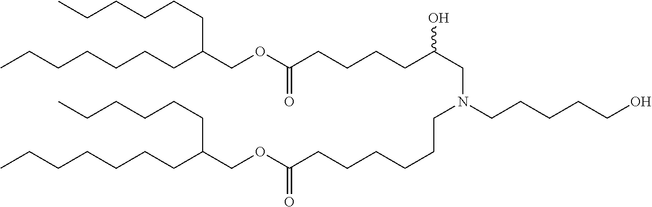 [SMILES: CCCCCCCC(CCCCCC)COC(=O)CCCCCCN(CCCCCO)CC(O)CCCCC(=O)OCC(CCCCCC)CCCCCCC] |
| 133 | 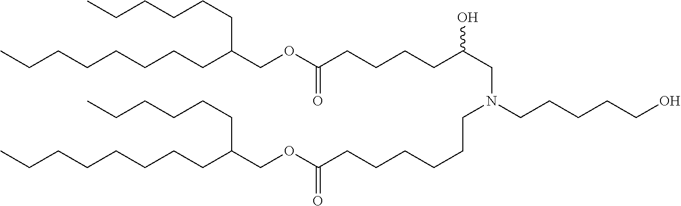 [SMILES: CCCCCCCCC(CCCCCC)COC(=O)CCCCCCN(CCCCCO)CC(O)CCCCC(=O)OCC(CCCCCC)CCCCCCCC] |
| 134 | 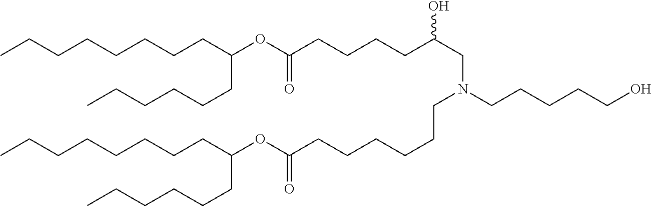 [SMILES: CCCCCCCCC(CCCCCC)OC(=O)CCCCCCN(CCCCCO)CC(O)CCCCC(=O)OC(CCCCCC)CCCCCCCC] |
| 135 | 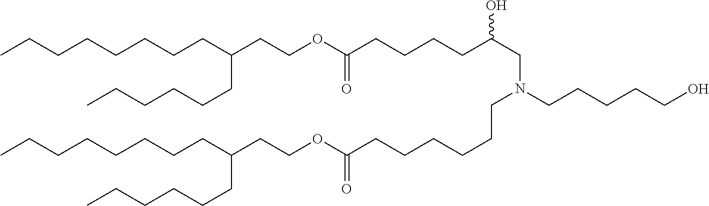 [SMILES: CCCCCCCCC(CCCCCC)CCOC(=O)CCCCCCN(CCCCCO)CC(O)CCCCC(=O)OCCC(CCCCCC)CCCCCCCC] |
| 136 | 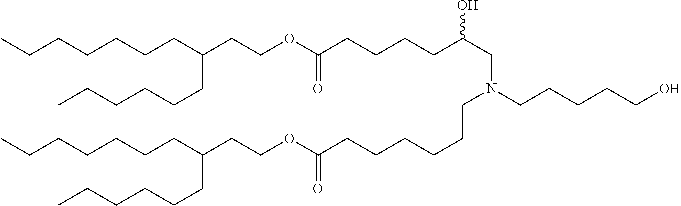 [SMILES: CCCCCCCC(CCCCCC)CCOC(=O)CCCCCCN(CCCCCO)CC(O)CCCCC(=O)OCCC(CCCCCC)CCCCCCC] |
| 137 | 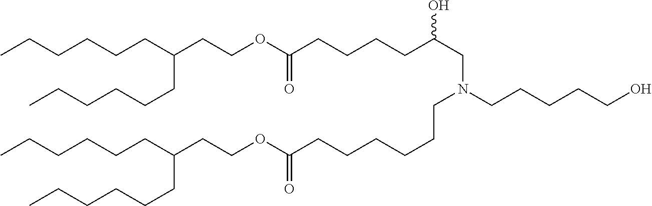 [SMILES: CCCCCCC(CCCCCC)CCOC(=O)CCCCCCN(CCCCCO)CC(O)CCCCC(=O)OCCC(CCCCCC)CCCCCC] |
| 138 | 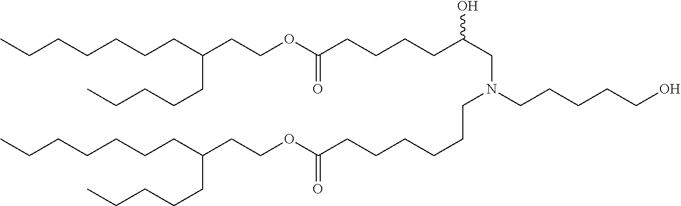 [SMILES: CCCCCCCC(CCCCC)CCOC(=O)CCCCCCN(CCCCCO)CC(O)CCCCC(=O)OCCC(CCCCC)CCCCCCC] |
| 139 | 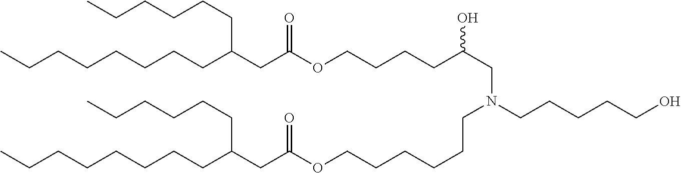 [SMILES: CCCCCCCCC(CCCCCC)CC(=O)OCCCCCCN(CCCCCO)CC(O)CCCCOC(=O)CC(CCCCCC)CCCCCCCC] |
| 140 | 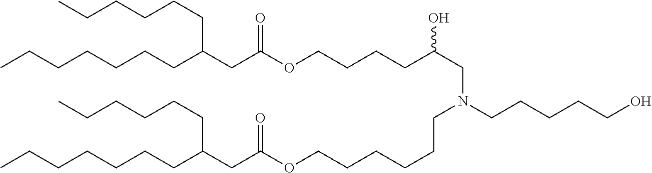 [SMILES: CCCCCCCC(CCCCCC)CC(=O)OCCCCCCN(CCCCCO)CC(O)CCCCOC(=O)CC(CCCCCC)CCCCCCC] |
| 141 | 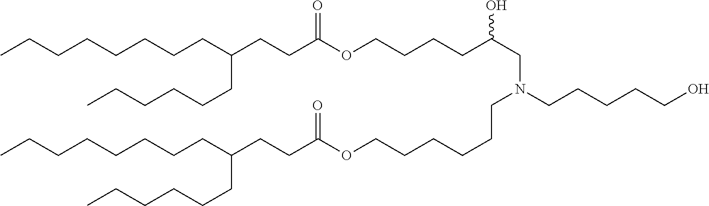 [SMILES: CCCCCCCCC(CCCCCC)CCC(=O)OCCCCCCN(CCCCCO)CC(O)CCCCOC(=O)CCC(CCCCCC)CCCCCCCC] |
| 142 | 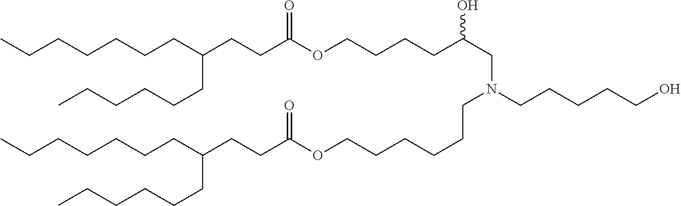 [SMILES: CCCCCCCC(CCCCCC)CCC(=O)OCCCCCCN(CCCCCO)CC(O)CCCCOC(=O)CCC(CCCCCC)CCCCCCC] |
| 143 | 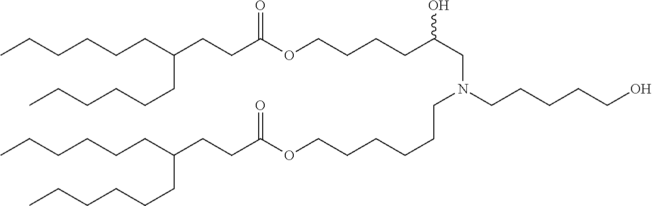 [SMILES: CCCCCCC(CCCCCC)CCC(=O)OCCCCCCN(CCCCCO)CC(O)CCCCOC(=O)CCC(CCCCCC)CCCCCC] |
| 144 | 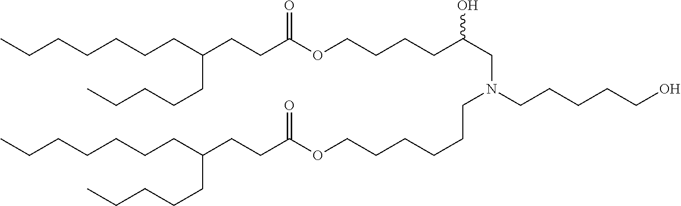 [SMILES: CCCCCCCC(CCCCC)CCC(=O)OCCCCCCN(CCCCCO)CC(O)CCCCOC(=O)CCC(CCCCC)CCCCCCC] |
| 145 | 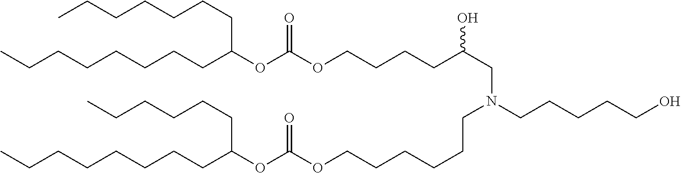 [SMILES: CCCCCCCCC(CCCCCC)OC(=O)OCCCCCCN(CCCCCO)CC(O)CCCCOC(=O)OC(CCCCCCC)CCCCCCCC] |
| 146 | 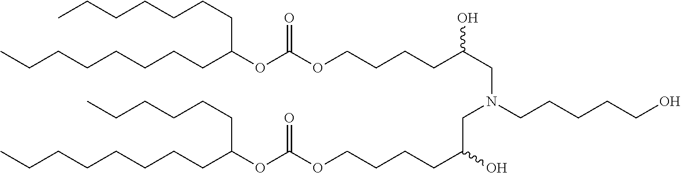 [SMILES: CCCCCCCCC(CCCCCC)OC(=O)OCCCCC(O)CN(CCCCCO)CC(O)CCCCOC(=O)OC(CCCCCCC)CCCCCCCC] |
| 147 | 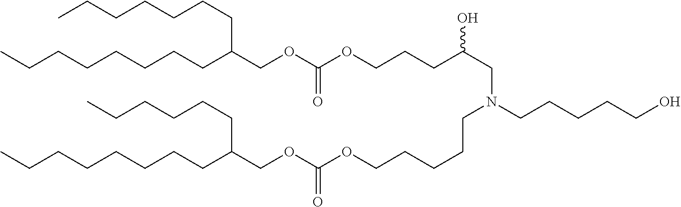 [SMILES: CCCCCCCCC(CCCCCC)COC(=O)OCCCCCN(CCCCCO)CC(O)CCCOC(=O)OCC(CCCCCCC)CCCCCCCC] |
| 148 | 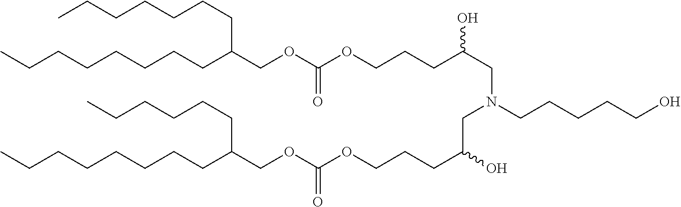 [SMILES: CCCCCCCCC(CCCCCC)COC(=O)OCCCC(O)CN(CCCCCO)CC(O)CCCOC(=O)OCC(CCCCCCC)CCCCCCCC] |
| 149 | 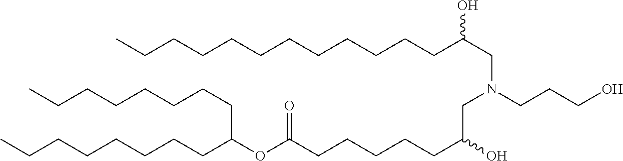 [SMILES: CCCCCCCCCCCCC(O)CN(CCCO)CC(O)CCCCCC(=O)OC(CCCCCCCC)CCCCCCCC] |
| 150 | 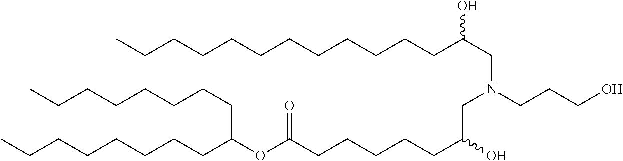 [SMILES: CCCCCCCCCCCCC(O)CN(CCCO)CC(O)CCCCCC(=O)OC(CCCCCCCC)CCCCCCCC] |
| 151 | 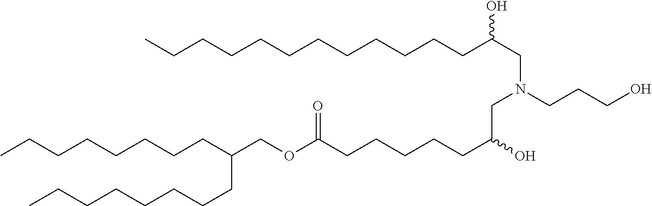 [SMILES: CCCCCCCCCCCCC(O)CN(CCCO)CC(O)CCCCCC(=O)OCC(CCCCCCCC)CCCCCCCC] |
| 152 | 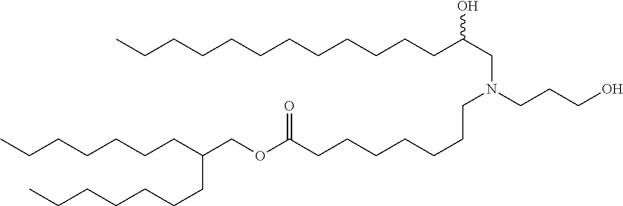 [SMILES: CCCCCCCCCCCCC(O)CN(CCCO)CCCCCCCC(=O)OCC(CCCCCCC)CCCCCCC] |
| 153 | 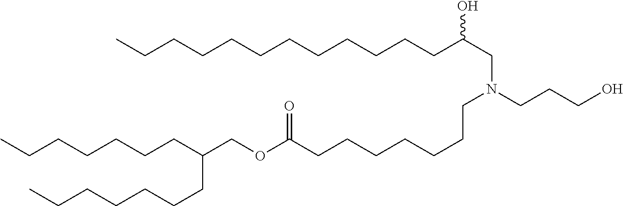 [SMILES: CCCCCCCCCCCCC(O)CN(CCCO)CCCCCCCC(=O)OCC(CCCCCCC)CCCCCCC] |
| 154 | 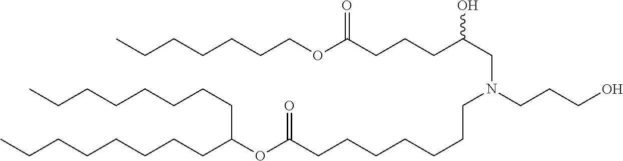 [SMILES: CCCCCCCCC(CCCCCCCC)OC(=O)CCCCCCCN(CCCO)CC(O)CCCC(=O)OCCCCCCC] |
| 155 | 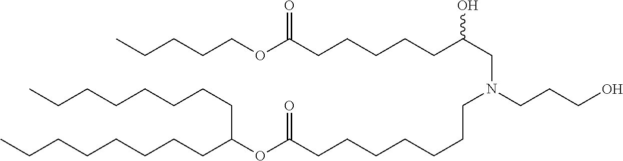 [SMILES: CCCCCCCCC(CCCCCCCC)OC(=O)CCCCCCCN(CCCO)CC(O)CCCCCC(=O)OCCCCC] |
| 156 | 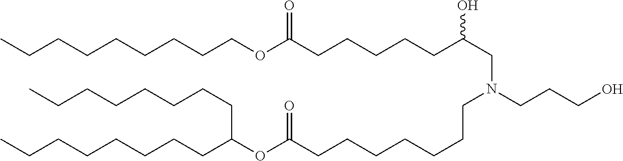 [SMILES: CCCCCCCCCOC(=O)CCCCCC(O)CN(CCCO)CCCCCCCC(=O)OC(CCCCCCCC)CCCCCCCC] |
| 157 | 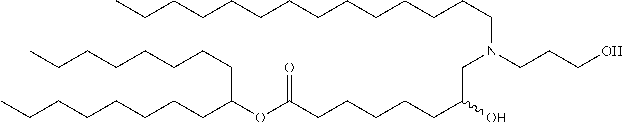 [SMILES: CCCCCCCCCCCCCCN(CCCO)CC(O)CCCCCC(=O)OC(CCCCCCCC)CCCCCCCC] |
| 158 | 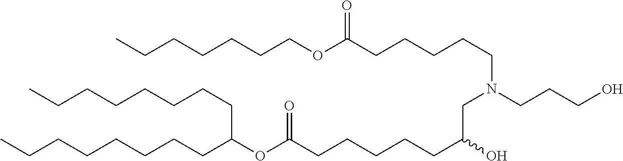 [SMILES: CCCCCCCCC(CCCCCCCC)OC(=O)CCCCCC(O)CN(CCCO)CCCCCC(=O)OCCCCCCC] |
| 159 | 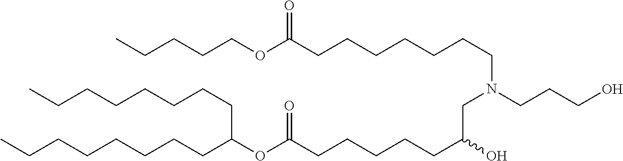 [SMILES: CCCCCCCCC(CCCCCCCC)OC(=O)CCCCCC(O)CN(CCCO)CCCCCCCC(=O)OCCCCC] |
| 160 | 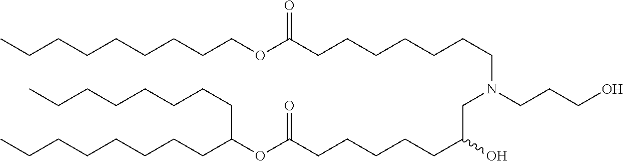 [SMILES: CCCCCCCCCOC(=O)CCCCCCCN(CCCO)CC(O)CCCCCC(=O)OC(CCCCCCCC)CCCCCCCC] |
| 161 | 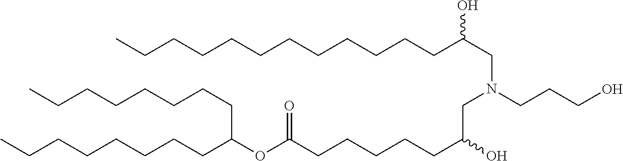 [SMILES: CCCCCCCCCCCCC(O)CN(CCCO)CC(O)CCCCCC(=O)OC(CCCCCCCC)CCCCCCCC] |
| 162 | 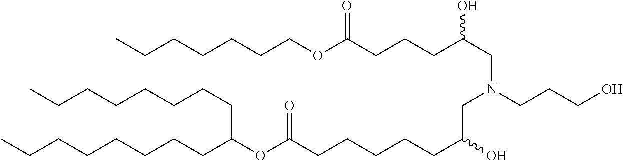 [SMILES: CCCCCCCCC(CCCCCCCC)OC(=O)CCCCCC(O)CN(CCCO)CC(O)CCCC(=O)OCCCCCCC] |
| 163 | 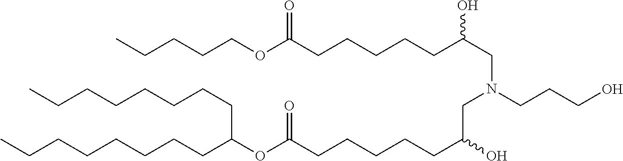 [SMILES: CCCCCCCCC(CCCCCCCC)OC(=O)CCCCCC(O)CN(CCCO)CC(O)CCCCCC(=O)OCCCCC] |
| 164 | 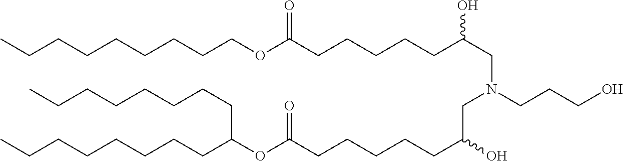 [SMILES: CCCCCCCCCOC(=O)CCCCCC(O)CN(CCCO)CC(O)CCCCCC(=O)OC(CCCCCCCC)CCCCCCCC] |
| 165 | 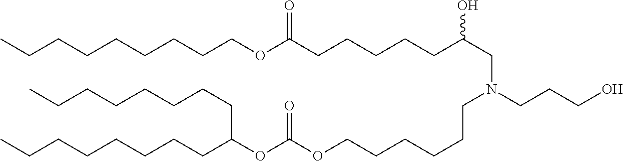 [SMILES: CCCCCCCCCOC(=O)CCCCCC(O)CN(CCCO)CCCCCCOC(=O)OC(CCCCCCCC)CCCCCCCC] |
| 166 | 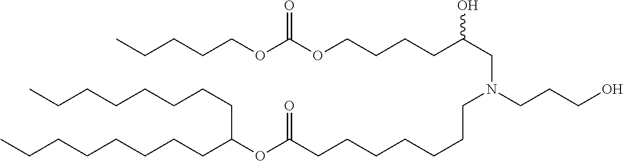 [SMILES: CCCCCCCCC(CCCCCCCC)OC(=O)CCCCCCCN(CCCO)CC(O)CCCCOC(=O)OCCCCC] |
| 167 | 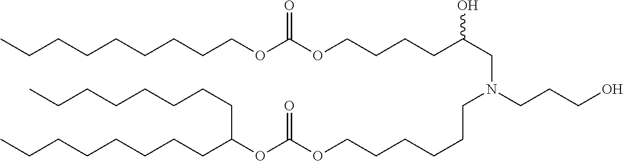 [SMILES: CCCCCCCCCOC(=O)OCCCCC(O)CN(CCCO)CCCCCCOC(=O)OC(CCCCCCCC)CCCCCCCC] |
| 168 | 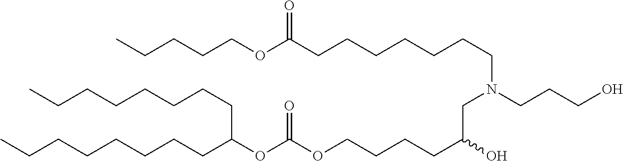 [SMILES: CCCCCCCCC(CCCCCCCC)OC(=O)OCCCCC(O)CN(CCCO)CCCCCCCC(=O)OCCCCC] |
| 169 | 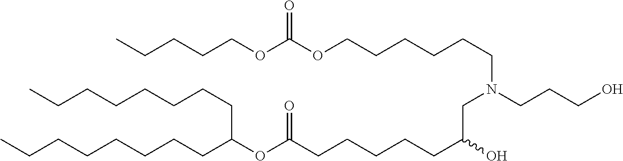 [SMILES: CCCCCCCCC(CCCCCCCC)OC(=O)CCCCCC(O)CN(CCCO)CCCCCCOC(=O)OCCCCC] |
| 170 | 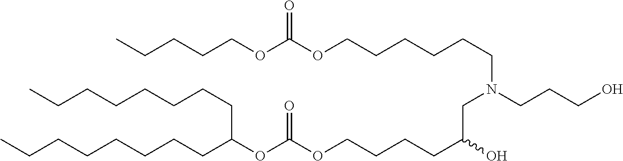 [SMILES: CCCCCCCCC(CCCCCCCC)OC(=O)OCCCCC(O)CN(CCCO)CCCCCCOC(=O)OCCCCC] |
| 171 | 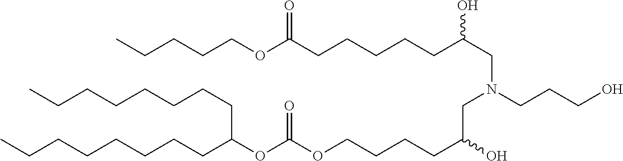 [SMILES: CCCCCCCCC(CCCCCCCC)OC(=O)OCCCCC(O)CN(CCCO)CC(O)CCCCCC(=O)OCCCCC] |
| 172 | 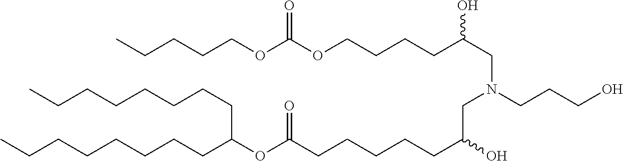 [SMILES: CCCCCCCCC(CCCCCCCC)OC(=O)CCCCCC(O)CN(CCCO)CC(O)CCCCOC(=O)OCCCCC] |
| 173 | 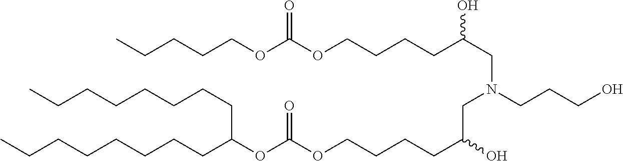 [SMILES: CCCCCCCCC(CCCCCCCC)OC(=O)OCCCCC(O)CN(CCCO)CC(O)CCCCOC(=O)OCCCCC] |
| 174 | 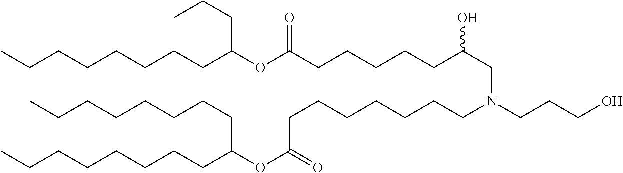 [SMILES: CCCCCCCCC(CCC)OC(=O)CCCCCC(O)CN(CCCO)CCCCCCCC(=O)OC(CCCCCCCC)CCCCCCCC] |
| 175 | 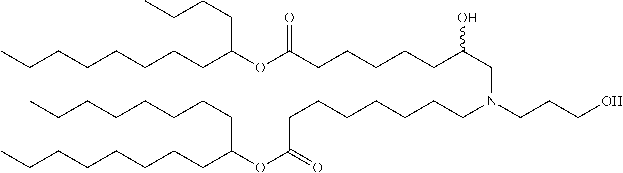 [SMILES: CCCCCCCCC(CCCC)OC(=O)CCCCCC(O)CN(CCCO)CCCCCCCC(=O)OC(CCCCCCCC)CCCCCCCC] |
| 176 | 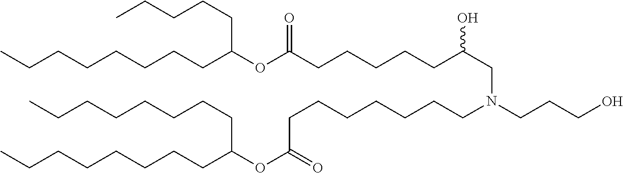 [SMILES: CCCCCCCCC(CCCCC)OC(=O)CCCCCC(O)CN(CCCO)CCCCCCCC(=O)OC(CCCCCCCC)CCCCCCCC] |
| 177 | 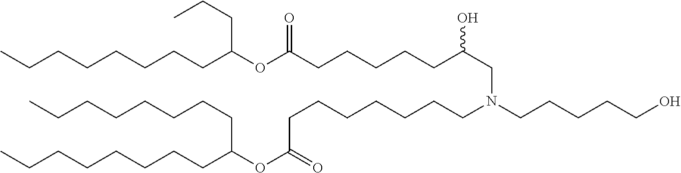 [SMILES: CCCCCCCCC(CCC)OC(=O)CCCCCC(O)CN(CCCCCO)CCCCCCCC(=O)OC(CCCCCCCC)CCCCCCCC] |
| 178 | 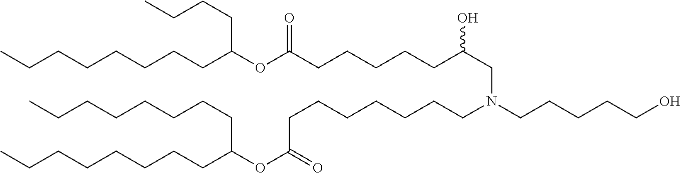 [SMILES: CCCCCCCCC(CCCC)OC(=O)CCCCCC(O)CN(CCCCCO)CCCCCCCC(=O)OC(CCCCCCCC)CCCCCCCC] |
| 179 | 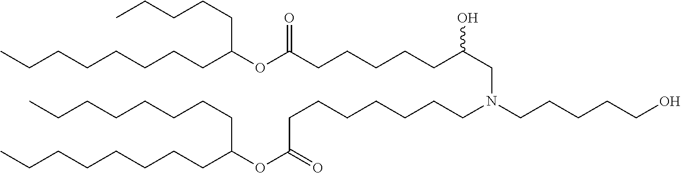 [SMILES: CCCCCCCCC(CCCCC)OC(=O)CCCCCC(O)CN(CCCCCO)CCCCCCCC(=O)OC(CCCCCCCC)CCCCCCCC] |
| 180 | 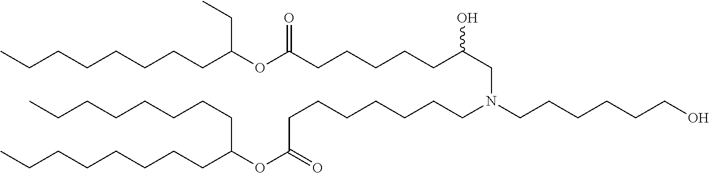 [SMILES: CCCCCCCCC(CC)OC(=O)CCCCCC(O)CN(CCCCCCO)CCCCCCCC(=O)OC(CCCCCCCC)CCCCCCCC] |
| 181 | 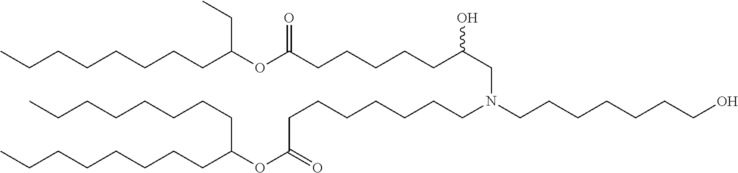 [SMILES: CCCCCCCCC(CC)OC(=O)CCCCCC(O)CN(CCCCCCCO)CCCCCCCC(=O)OC(CCCCCCCC)CCCCCCCC] |
| 182 | 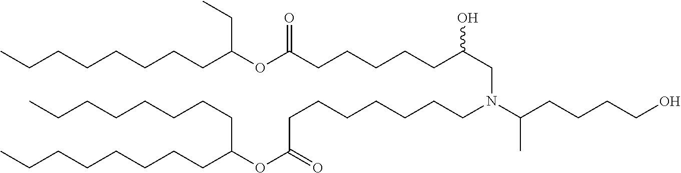 [SMILES: CCCCCCCCC(CC)OC(=O)CCCCCC(O)CN(CCCCCCCC(=O)OC(CCCCCCCC)CCCCCCCC)C(C)CCCCO] |
| 183 | 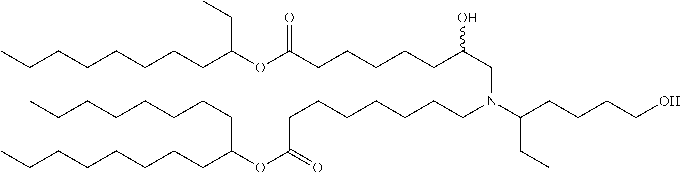 [SMILES: CCCCCCCCC(CC)OC(=O)CCCCCC(O)CN(CCCCCCCC(=O)OC(CCCCCCCC)CCCCCCCC)C(CC)CCCCO] |
| 184 | 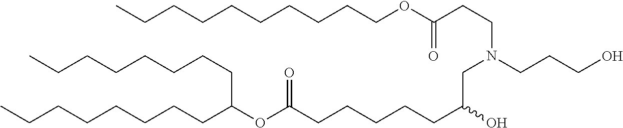 [SMILES: CCCCCCCCCCOC(=O)CCN(CCCO)CC(O)CCCCCC(=O)OC(CCCCCCCC)CCCCCCCC] |
| 185 | 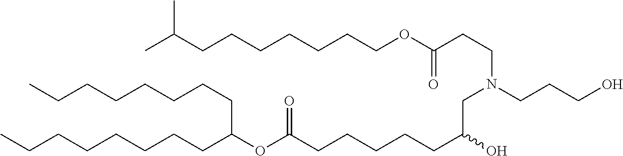 [SMILES: CCCCCCCCC(CCCCCCCC)OC(=O)CCCCCC(O)CN(CCCO)CCC(=O)OCCCCCCCC(C)C] |

In some embodiments, an ionizable lipid is a compound of Formula (15):

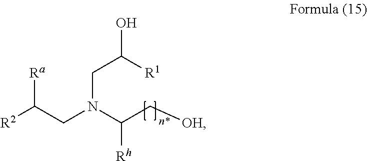

or is a pharmaceutically acceptable salt thereof, wherein:
n* is an integer from 1 to 7;
R^{a} is hydrogen or hydroxyl;
R^{h} is hydrogen or C₁-C₆ alkyl;
R¹ is C₁-C₃₀ alkyl or R¹*;
R² is C₁-C₃₀ alkyl or R²*;
R¹* and R²* are independently selected from:

—(CH₂)_{q}C(O)O(CH₂)ᵣC(R⁸)(R⁹)(R¹⁰),

—(CH₂)_{q}OC(O)(CH₂)ᵣC(R⁸)(R⁹)(R¹⁰), and

—(CH₂)_{q}OC(O)O(CH₂)ᵣC(R⁸)(R⁹)(R¹⁰);
wherein:
   q is an integer from 0 to 12,
   r is an integer from 0 to 6, wherein at least one occurrence of r is not 0;
   R⁸ is H or R¹¹;
   R⁹, R¹⁰, and R¹¹ are each independently C₁-C₂₀ alkyl or C₂-C₂₀-alkenyl; and
wherein (i) R¹ is R¹*, (ii) R² is R²*, or (iii) R¹ is R¹* and R² is R²*;

In some embodiments of Formula (15), R^{a} is hydrogen and the ionizable lipid is of Formula (16):

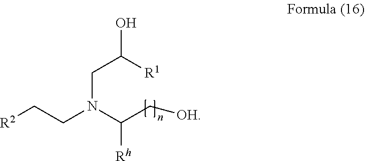

or is a pharmaceutically acceptable salt thereof, wherein:
n* is an integer from 1 to 7.

In some embodiments of Formula (16), the ionizable lipid is of Formula (17):

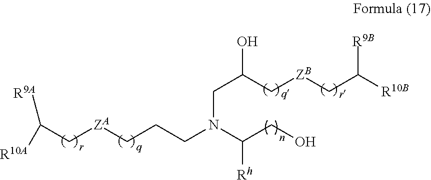

or a pharmaceutically acceptable salt thereof, wherein:
n is an integer from 1 to 7;
q and q′ are each independently integers from 0 to 12;
r and r′ are each independently integers from 0 to 6, wherein at least one of r or r′ is not 0;
Z^{A} and Z^{B} are each independently selected from \hat{ }—C(O)O—, \hat{ }—OC(O), and —OC(O)O—; where \hat{ } denotes the attachment point to —(CH₂)_{q}— or —(CH₂)_{q′}—; and
R^{9A}, R^{9B}, R^{10A}, and R^{10B} are each independently C₁-C₂₀ alkyl or C₂-C₂₀ alkenyl.

In some embodiments of Formula (17), Z^{A} and Z^{B} are \hat{ }—C(O)O—, and the ionizable lipid is of Formula (17a-1)

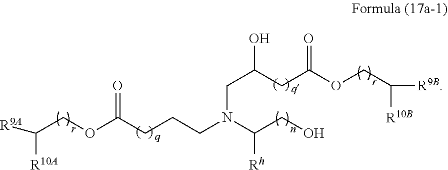

In some embodiments of Formula (17), Z^{A} and Z^{B} are \hat{ }—OC(O)—, and the ionizable lipid is of Formula (17a-2)

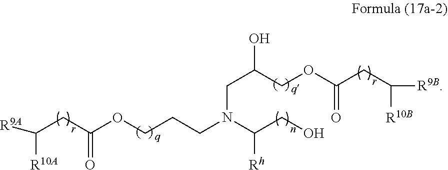

In some embodiments of Formula (17), Z^{A} and Z^{B} are —O(C)(O)O—, and the ionizable lipid is represented by Formula (17a-3):

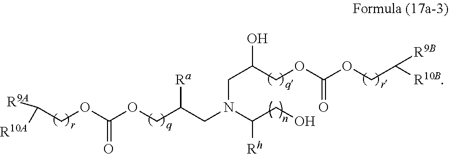

In some embodiments of Formula (15), R^{a} is hydroxyl and the ionizable lipid is of Formula (18):

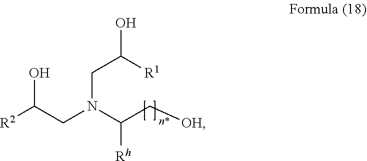

or is a pharmaceutically acceptable salt thereof, wherein:
n* is an integer from 1 to 7;
R^{h} is hydrogen or C₁-C₆ alkyl;
R¹ is C₁-C₃₀ alkyl or R¹*;
R² is C₁-C₃₀ alkyl or R²*;
R¹* and R²* are independently selected from:

—(CH₂)_{q}C(O)O(CH₂)ᵣC(R⁸)(R⁹)(R¹⁰),

—(CH₂)_{q}OC(O)(CH₂)ᵣC(R⁸)(R⁹)(R¹⁰), and

—(CH₂)_{q}OC(O)O(CH₂)ᵣC(R⁸)(R⁹)(R¹⁰);
wherein:
q is an integer from 0 to 12,
r is an integer from 0 to 6, wherein at least one occurrence of r is not 0;
R⁸ is hydrogen or R¹¹;
R⁹, R¹⁰, and R¹¹ are each independently C₁-C₂₀ alkyl or C₂-C₂₀-alkenyl;
wherein (i) R¹ is R¹*, (ii) R² is R²*, or (iii) R¹ is R¹* and R² is R²*; and
wherein, for (iii), (a) R¹* and R²* are different or (b) R⁹ and R¹⁰ have different numbers of carbon atoms for at least one of R¹* and R²*.

In some embodiments of Formula (18), the ionizable lipid of is of Formula (19):

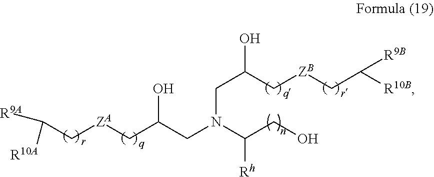

or is a pharmaceutically acceptable salt thereof, wherein:
   n is an integer from 1 to 7;
   q and q′ are each independently integers from 0 to 12;
   r and r′ are each independently integers from 0 to 6, wherein at least one of r
   or r′ is not 0;
   Z^{A} and Z^{B} are each independently selected from \hat{ }—C(O)O—, \hat{ }—OC(O), and —OC(O)O—; where \hat{ } denotes the attachment point to —(CH₂)_{q}— or —(CH₂)_{q′}; —and
   R^{9A}, R^{9B}, R^{10A}, and R^{10B} are each independently C₁-C₂₀ alkyl or C₂-C₂₀ alkenyl.

In some embodiments of Formula (19), Z^{A} and Z^{B} are \hat{ }—C(O)O—, and the ionizable lipid is of Formula (19a-1):

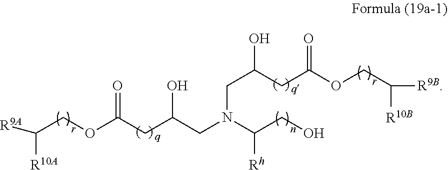

In some embodiments of Formula (19), Z^{A} and Z^{B} are \hat{ }—OC(O)—, and the ionizable lipid is of Formula (19a-2):

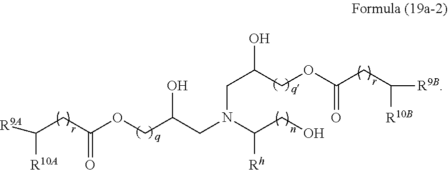

In some embodiments of Formula (19), Z^{A} and Z^{B} are —O(C)(O)O—, and the ionizable lipid is represented by Formula (19a-3):

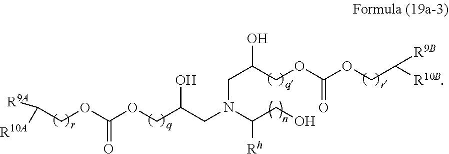

In some embodiments of Formula (15), R¹ is C₁-C₃₀ alkyl, and the ionizable lipid is of Formula (20):

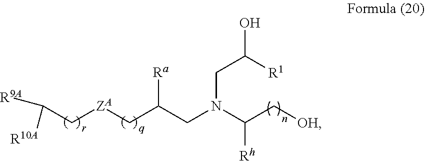

or is a pharmaceutically acceptable salt thereof, wherein:
Z^{A} is selected from \hat{ }—C(O)O—, \hat{ }—OC(O)—, and —OC(O)O—; where \hat{ } denotes the attachment point to —(CH₂)_{q}—;
R^{9A} and R^{10A} are each independently C₁-C₂₀ alkyl or C₂-C₂₀ alkenyl;
n is an integer from 1 to 7;
q is an integer from 0 to 12; and
r is an integer from 1 to 6.

In some embodiments of Formula (20), Z^{A} is \hat{ }—C(O)O—, and the ionizable lipid is of Formula (20a-1):

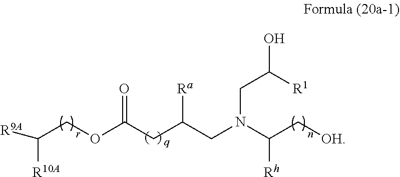

In some embodiments of Formula (20), Z^{A} is \hat{ }—OC(O)—, and the ionizable lipid is of Formula (20a-2):

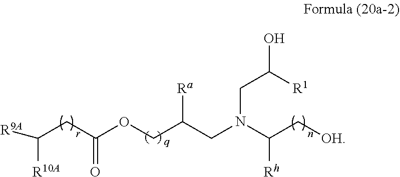

In some embodiments of Formula (20), Z^{A} is —OC(O)O—, and the ionizable lipid is of Formula (20a-3):

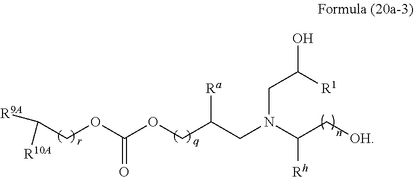

In some embodiments of Formula (15), R² is C₁-C₃₀ alkyl, and the ionizable lipid is of Formula (21):

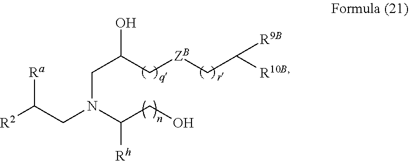

or is a pharmaceutically acceptable salt thereof, wherein:
Z^{B} is selected from \hat{ }—C(O)O—, \hat{ }—OC(O)—, and —OC(O)O—; where \hat{ } denotes the attachment point to —(CH₂)_{q′}—;
R^{9B} and R^{10B} are each independently C₁-C₂₀ alkyl or C₂-C₂₀ alkenyl;
n is an integer from 1 to 7;
q′ is an integer from 0 to 12; and
r′ is an integer from 1 to 6.

In some embodiments of Formula (21), Z^{B} is \hat{ }—C(O)O—, and the ionizable lipid is of Formula (21a-1):

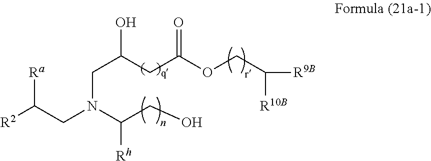

In some embodiments of Formula (21), Z^{B} is \hat{ }—OC(O)—, and the ionizable lipid is of Formula (21a-2):

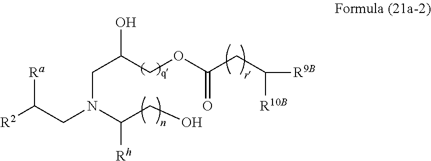

In some embodiments of Formula (21), Z^{B} is —OC(O)O—, and the ionizable lipid is of Formula (21a-3):

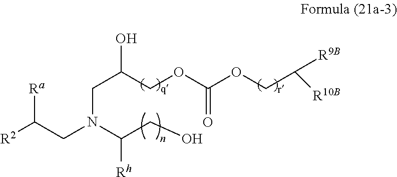

In some embodiments, an ionizable lipid is selected from the table below:

| Ionizable | |
|---|---|
| lipid | |
| number | Structure |
| 1-a | 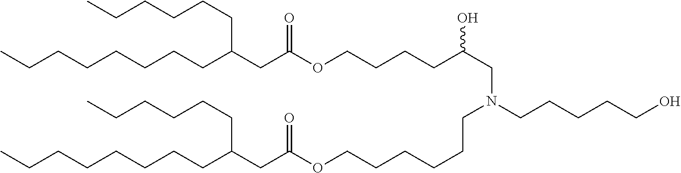 [SMILES: CCCCCCCCC(CCCCCC)CC(=O)OCCCCCCN(CCCCCO)CC(O)CCCCOC(=O)CC(CCCCCC)CCCCCCCC] |
| 1-b | 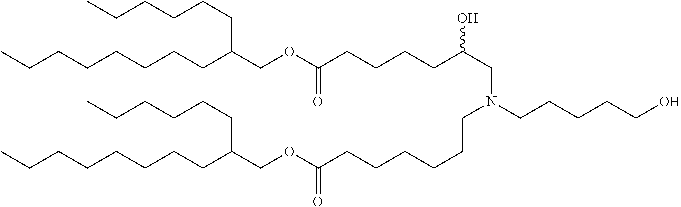 [SMILES: CCCCCCCCC(CCCCCC)COC(=O)CCCCCCN(CCCCCO)CC(O)CCCCC(=O)OCC(CCCCCC)CCCCCCCC] |
| 1-c | 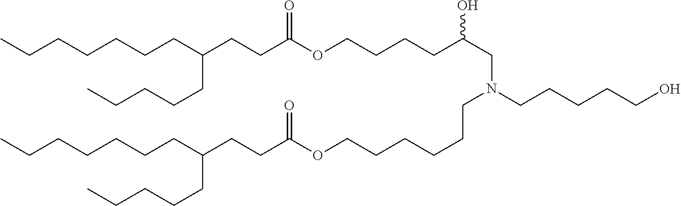 [SMILES: CCCCCCCC(CCCCC)CCC(=O)OCCCCCCN(CCCCCO)CC(O)CCCCOC(=O)CCC(CCCCC)CCCCCCC] |
| 1-d | 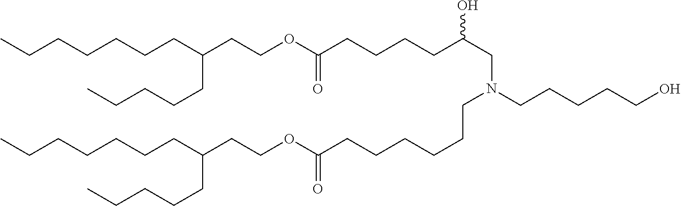 [SMILES: CCCCCCCC(CCCCC)CCOC(=O)CCCCCCN(CCCCCO)CC(O)CCCCC(=O)OCCC(CCCCC)CCCCCCC] |
| 1-e | 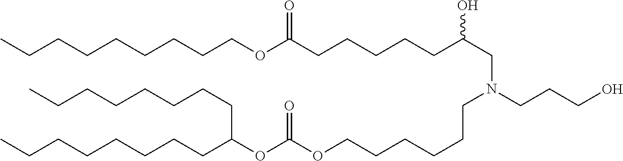 [SMILES: CCCCCCCCCOC(=O)CCCCCC(O)CN(CCCO)CCCCCCOC(=O)OC(CCCCCCCC)CCCCCCCC] |
| 1-f | 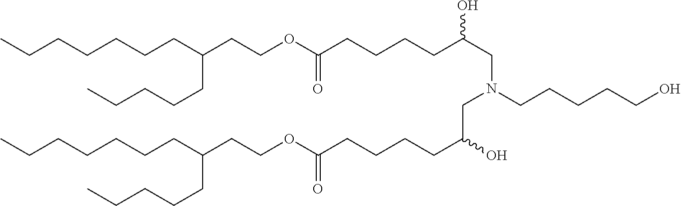 [SMILES: CCCCCCCC(CCCCC)CCOC(=O)CCCCC(O)CN(CCCCCO)CC(O)CCCCC(=O)OCCC(CCCCC)CCCCCCC] |
| 1-g | 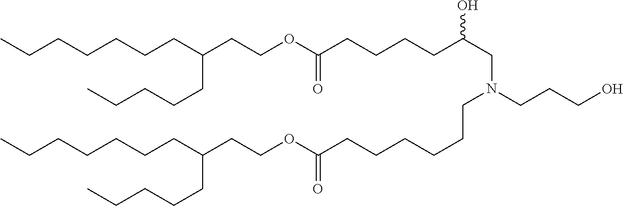 [SMILES: CCCCCCCC(CCCCC)CCOC(=O)CCCCCCN(CCCO)CC(O)CCCCC(=O)OCCC(CCCCC)CCCCCCC] |
| 1-h | 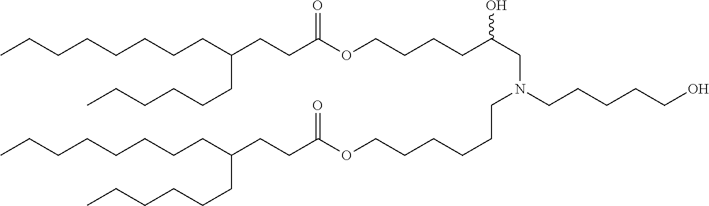 [SMILES: CCCCCCCCC(CCCCCC)CCC(=O)OCCCCCCN(CCCCCO)CC(O)CCCCOC(=O)CCC(CCCCCC)CCCCCCCC] |
| 1-i | 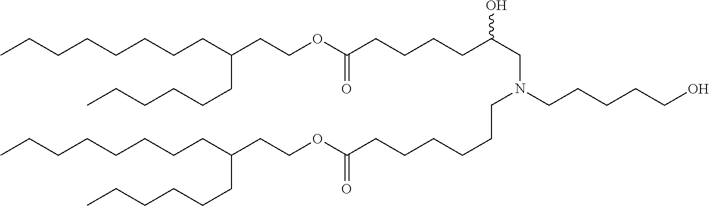 [SMILES: CCCCCCCCC(CCCCCC)CCOC(=O)CCCCCCN(CCCCCO)CC(O)CCCCC(=O)OCCC(CCCCCC)CCCCCCCC] |
| 1-j | 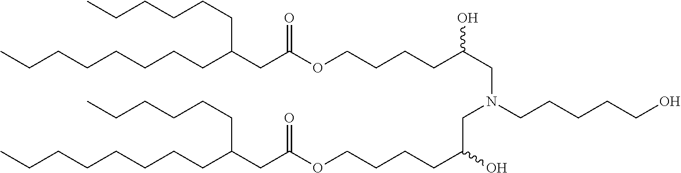 [SMILES: CCCCCCCCC(CCCCCC)CC(=O)OCCCCC(O)CN(CCCCCO)CC(O)CCCCOC(=O)CC(CCCCCC)CCCCCCCC] |
| 1-k | 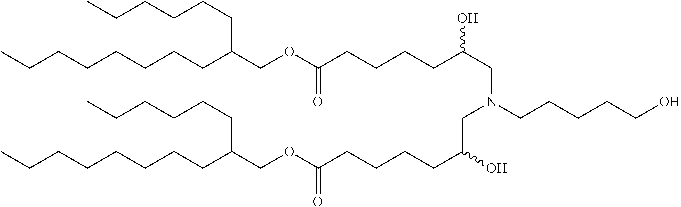 [SMILES: CCCCCCCCC(CCCCCC)COC(=O)CCCCC(O)CN(CCCCCO)CC(O)CCCCC(=O)OCC(CCCCCC)CCCCCCCC] |
| 1-l | 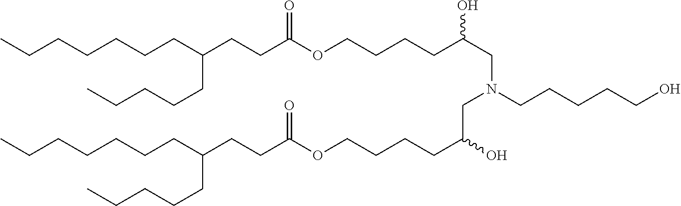 [SMILES: CCCCCCCC(CCCCC)CCC(=O)OCCCCC(O)CN(CCCCCO)CC(O)CCCCOC(=O)CCC(CCCCC)CCCCCCC] |
| 1-m | 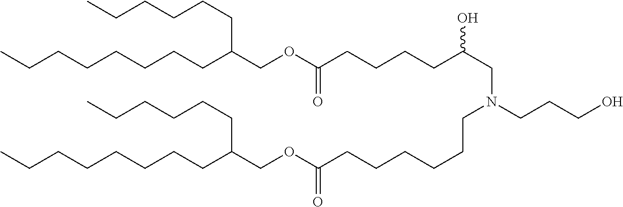 [SMILES: CCCCCCCCC(CCCCCC)COC(=O)CCCCCCN(CCCO)CC(O)CCCCC(=O)OCC(CCCCCC)CCCCCCCC] |
| 1-m | 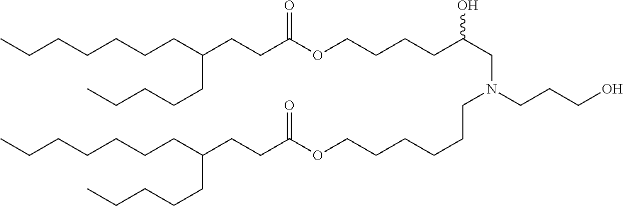 [SMILES: CCCCCCCC(CCCCC)CCC(=O)OCCCCCCN(CCCO)CC(O)CCCCOC(=O)CCC(CCCCC)CCCCCCC] |
| 1-n | 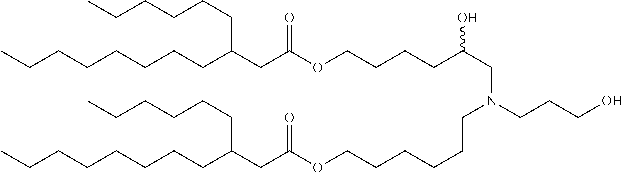 [SMILES: CCCCCCCCC(CCCCCC)CC(=O)OCCCCCCN(CCCO)CC(O)CCCCOC(=O)CC(CCCCCC)CCCCCCCC] |
| 1-o | 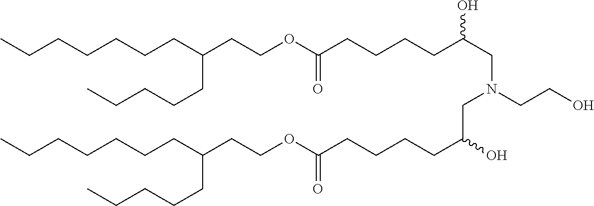 [SMILES: CCCCCCCC(CCCCC)CCOC(=O)CCCCC(O)CN(CCO)CC(O)CCCCC(=O)OCCC(CCCCC)CCCCCCC] |
| 1-p | 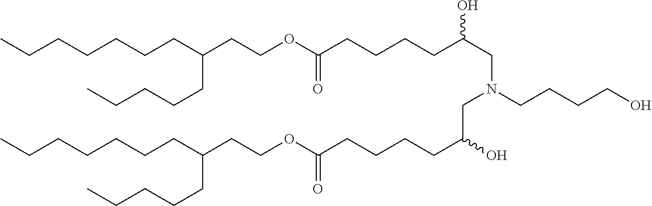 [SMILES: CCCCCCCC(CCCCC)CCOC(=O)CCCCC(O)CN(CCCCO)CC(O)CCCCC(=O)OCCC(CCCCC)CCCCCCC] |
| 1-q | 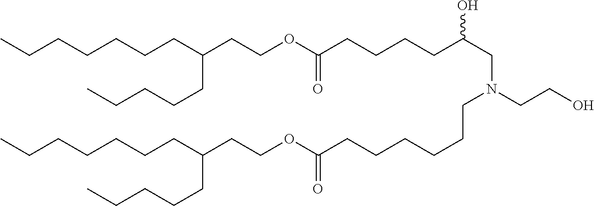 [SMILES: CCCCCCCC(CCCCC)CCOC(=O)CCCCCCN(CCO)CC(O)CCCCC(=O)OCCC(CCCCC)CCCCCCC] |
| 1-r | 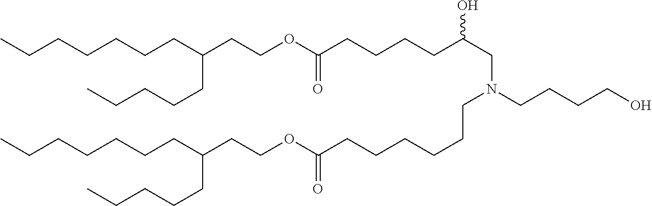 [SMILES: CCCCCCCC(CCCCC)CCOC(=O)CCCCCCN(CCCCO)CC(O)CCCCC(=O)OCCC(CCCCC)CCCCCCC] |
| 1-s | 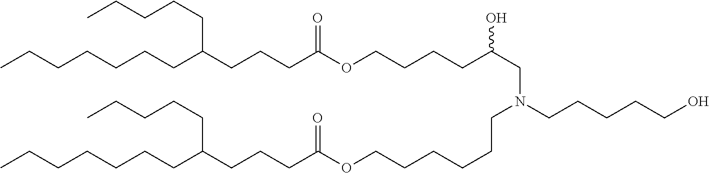 [SMILES: CCCCCCCC(CCCCC)CCCC(=O)OCCCCCCN(CCCCCO)CC(O)CCCCOC(=O)CCCC(CCCCC)CCCCCCC] |
| 1-t | 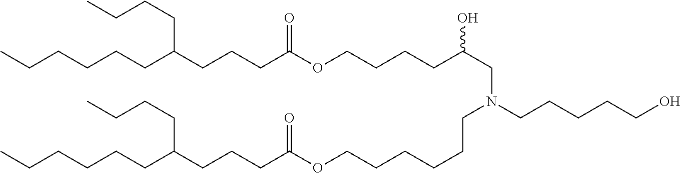 [SMILES: CCCCCCC(CCCC)CCCC(=O)OCCCCCCN(CCCCCO)CC(O)CCCCOC(=O)CCCC(CCCC)CCCCCC] |
| 1-u | 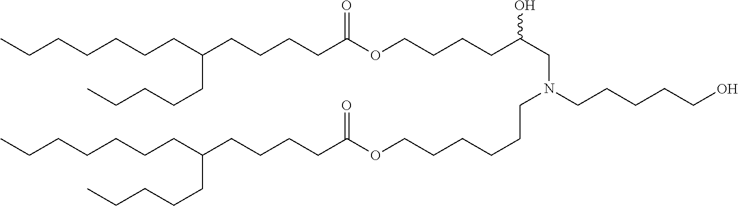 [SMILES: CCCCCCCC(CCCCC)CCCCC(=O)OCCCCCCN(CCCCCO)CC(O)CCCCOC(=O)CCCCC(CCCCC)CCCCCCC] |
| 1-v | 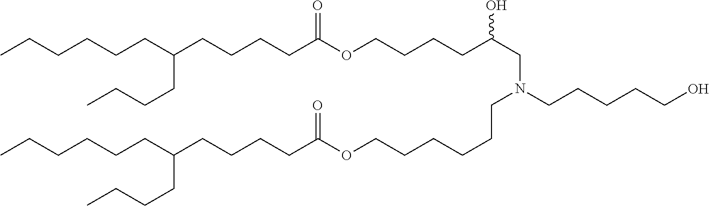 [SMILES: CCCCCCC(CCCC)CCCCC(=O)OCCCCCCN(CCCCCO)CC(O)CCCCOC(=O)CCCCC(CCCC)CCCCCC] |
| 1-w | 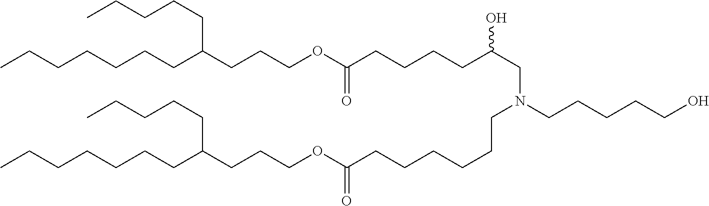 [SMILES: CCCCCCCC(CCCCC)CCCOC(=O)CCCCCCN(CCCCCO)CC(O)CCCCC(=O)OCCCC(CCCCC)CCCCCCC] |
| 1-x | 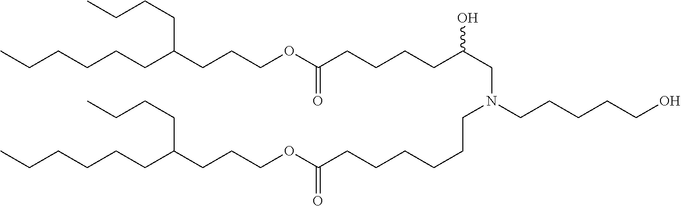 [SMILES: CCCCCCC(CCCC)CCCOC(=O)CCCCCCN(CCCCCO)CC(O)CCCCC(=O)OCCCC(CCCC)CCCCCC] |
| 1-y | 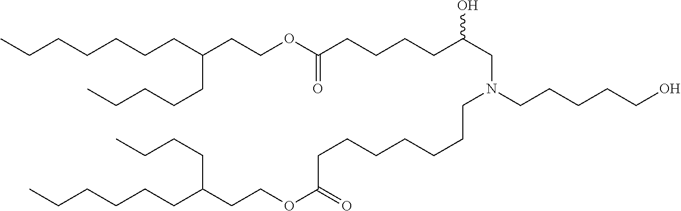 [SMILES: CCCCCCCC(CCCCC)CCOC(=O)CCCCC(O)CN(CCCCCO)CCCCCCCC(=O)OCCC(CCCC)CCCCCC] |
| 1-z | 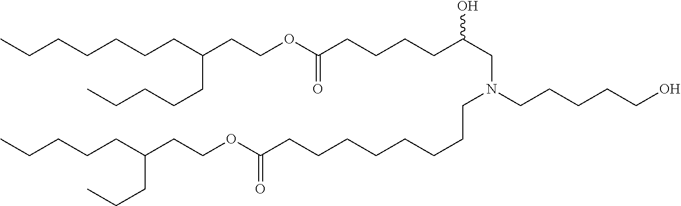 [SMILES: CCCCCCCC(CCCCC)CCOC(=O)CCCCC(O)CN(CCCCCO)CCCCCCCCC(=O)OCCC(CCC)CCCCC] |
| 1-aa | 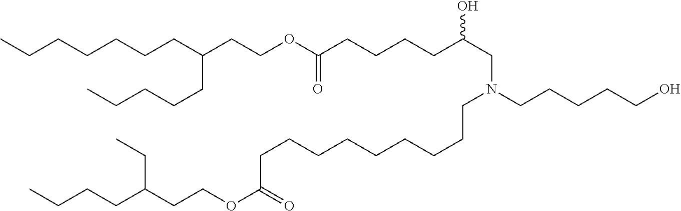 [SMILES: CCCCCCCC(CCCCC)CCOC(=O)CCCCC(O)CN(CCCCCO)CCCCCCCCCC(=O)OCCC(CC)CCCC] |
| 1-ab | 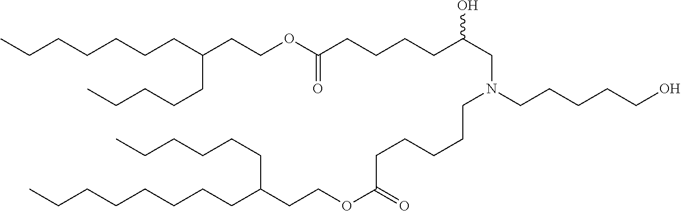 [SMILES: CCCCCCCCC(CCCCCC)CCOC(=O)CCCCCN(CCCCCO)CC(O)CCCCC(=O)OCCC(CCCCC)CCCCCCC] |
| 1-ac | 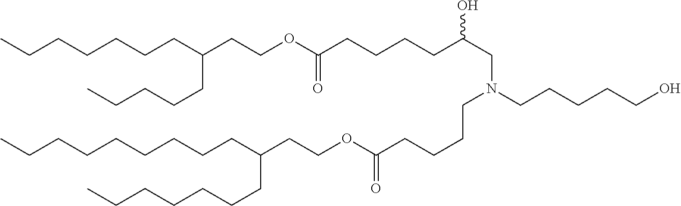 [SMILES: CCCCCCCCCC(CCCCCCC)CCOC(=O)CCCCN(CCCCCO)CC(O)CCCCC(=O)OCCC(CCCCC)CCCCCCC] |
| 1-ad | 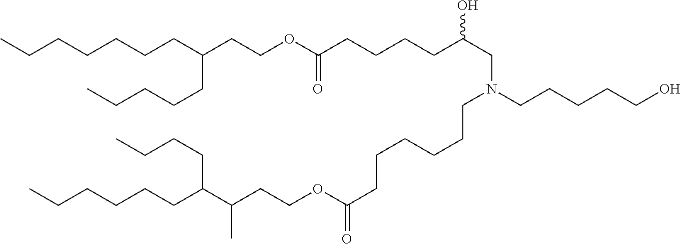 [SMILES: CCCCCCCC(CCCCC)CCOC(=O)CCCCC(O)CN(CCCCCO)CCCCCCC(=O)OCCC(C)C(CCCC)CCCCCC] |
| 1-ae | 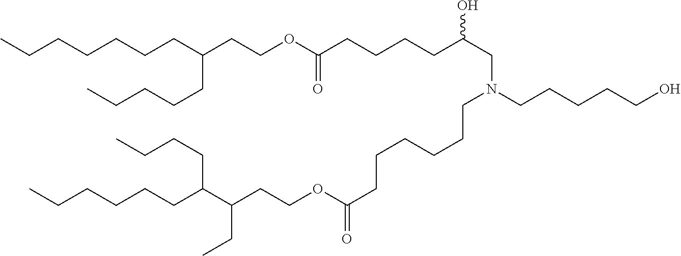 [SMILES: CCCCCCCC(CCCCC)CCOC(=O)CCCCC(O)CN(CCCCCO)CCCCCCC(=O)OCCC(CC)C(CCCC)CCCCCC] |
| 1-af | 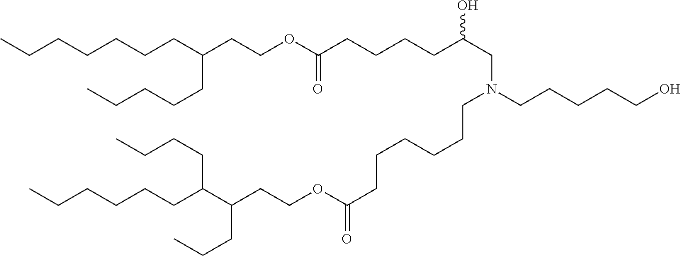 [SMILES: CCCCCCCC(CCCCC)CCOC(=O)CCCCC(O)CN(CCCCCO)CCCCCCC(=O)OCCC(CCC)C(CCCC)CCCCCC] |
| 1-ag | 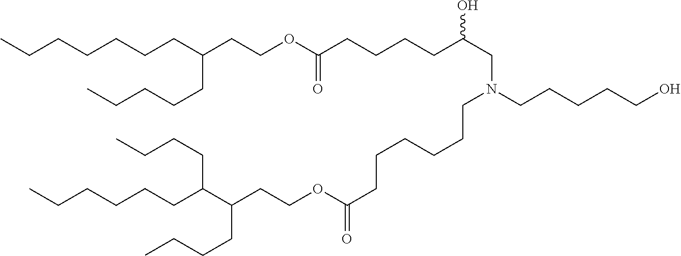 [SMILES: CCCCCCCC(CCCCC)CCOC(=O)CCCCC(O)CN(CCCCCO)CCCCCCC(=O)OCCC(CCCC)C(CCCC)CCCCCC] |
| 1-ah | 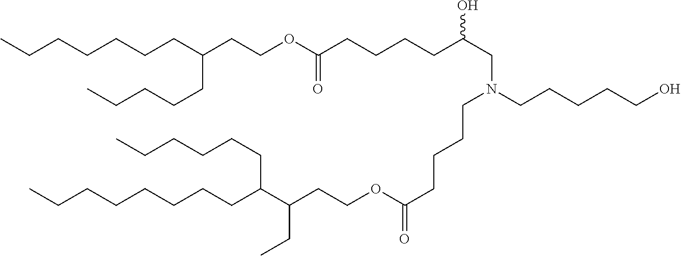 [SMILES: CCCCCCCCC(CCCCCC)C(CC)CCOC(=O)CCCCN(CCCCCO)CC(O)CCCCC(=O)OCCC(CCCCC)CCCCCCC] |
| 1-ai | 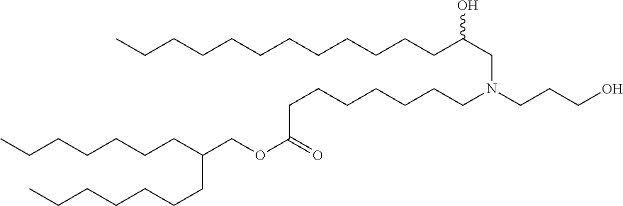 [SMILES: CCCCCCCCCCCCC(O)CN(CCCO)CCCCCCCC(=O)OCC(CCCCCCC)CCCCCCC] |
| 1-aj | 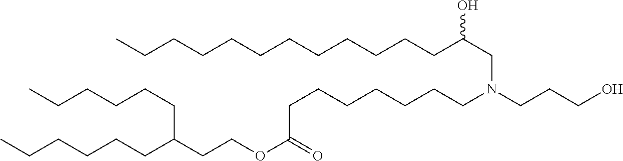 [SMILES: CCCCCCCCCCCCC(O)CN(CCCO)CCCCCCCC(=O)OCCC(CCCCCC)CCCCCC] |
| 1-ak | 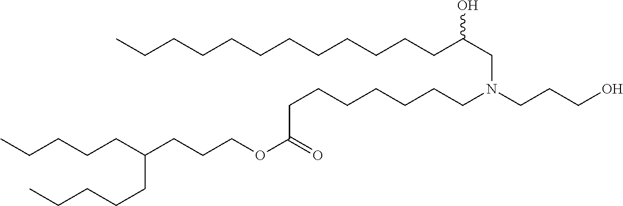 [SMILES: CCCCCCCCCCCCC(O)CN(CCCO)CCCCCCCC(=O)OCCCC(CCCCC)CCCCC] |

In some embodiments, an ionizable lipid of the present disclosure is represented by Formula (22):

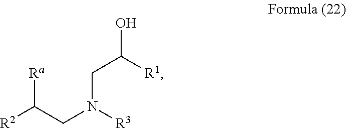

or is a pharmaceutically acceptable salt thereof, wherein:
R^{a} is hydrogen or hydroxyl;
R¹ is C₁-C₃₀ alkyl or R¹*;
R² is C₁-C₃₀ alkyl or R²*;
R¹* and R²* are independently selected from:

—(CH₂)_{q}C(O)O(CH₂)ᵣC(R⁴)(R⁵)(R⁶),

—(CH₂)_{q}OC(O)(CH₂)ᵣC(R⁴)(R⁵)(R⁶), and

—(CH₂)_{q}OC(O)O(CH₂)ᵣC(R⁴)(R⁵)(R⁶);
wherein:
   q is an integer from 0 to 12,
   r is an integer from 0 to 6, wherein at least one occurrence of r is not 0;
   R⁴ is hydrogen or R⁷;
   R⁵, R⁶, and R⁷ are each independently C₁-C₂₀ alkyl or C₂-C₂₀-alkenyl;
wherein (i) R¹ is R¹*, (ii) R² is R²*, or (iii) R¹ is R¹* and R² is R²*; and
R³ is L-R′, wherein L is linear or branched C₁-C₁₀ alkylene, and R′ is (i) mono- or bicyclic heterocyclyl or heteroaryl, such as imidazolyl, pyrazolyl, 1,2,4-triazolyl, or benzimidazolyl, each optionally substituted at one or more available carbon and nitrogen by C₁-C₆ alkyl, or (ii) R^{A}, R^{B}, or R^{C}, wherein
   R^{A} is selected from:

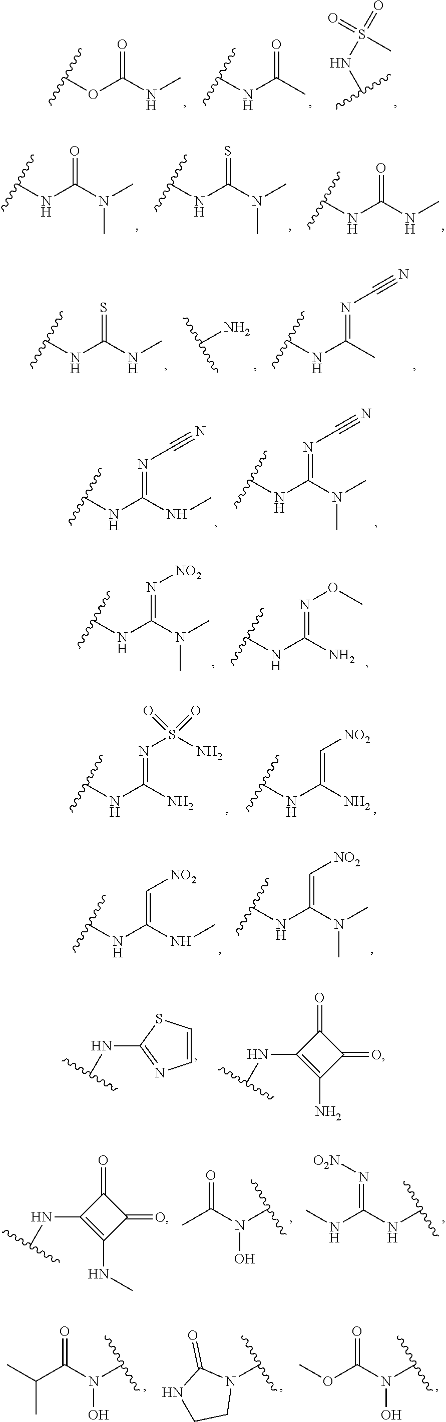

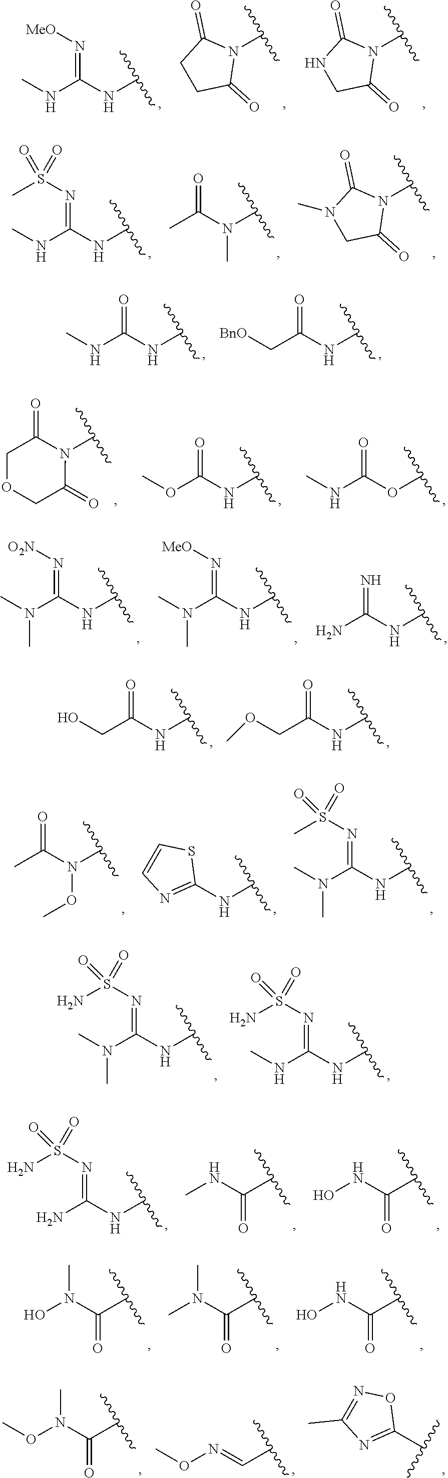

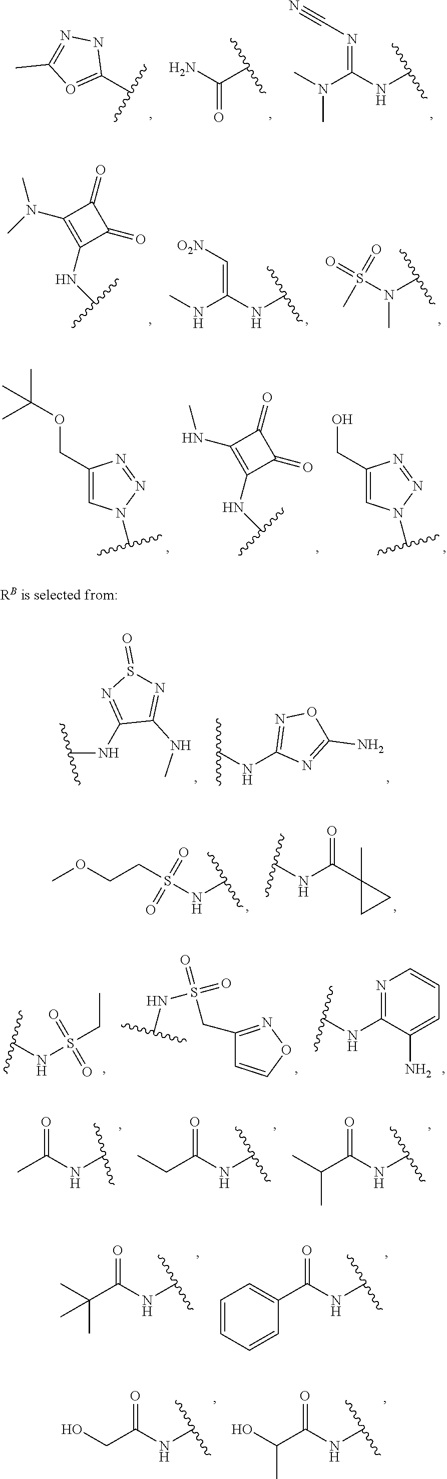

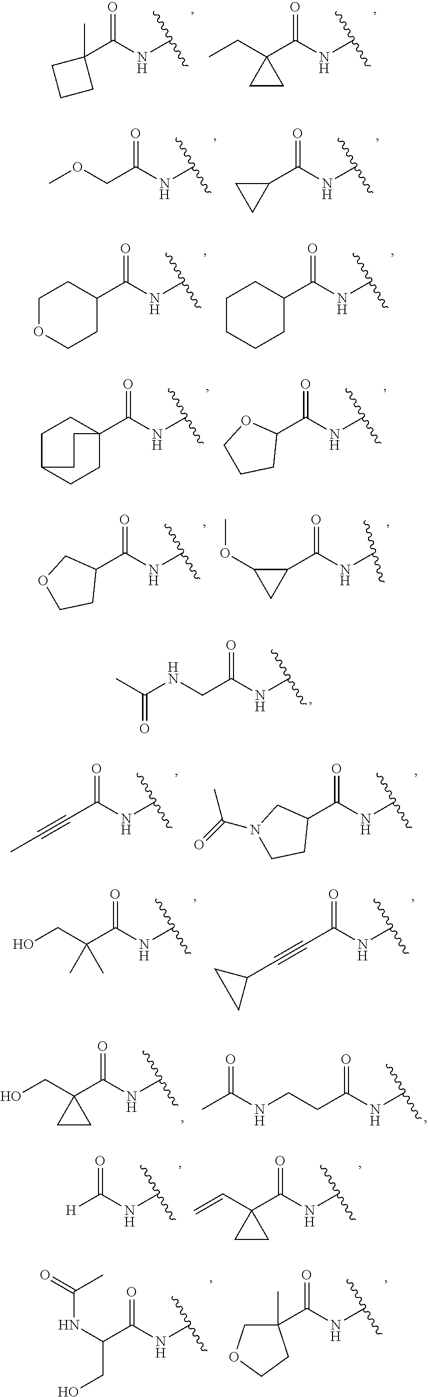

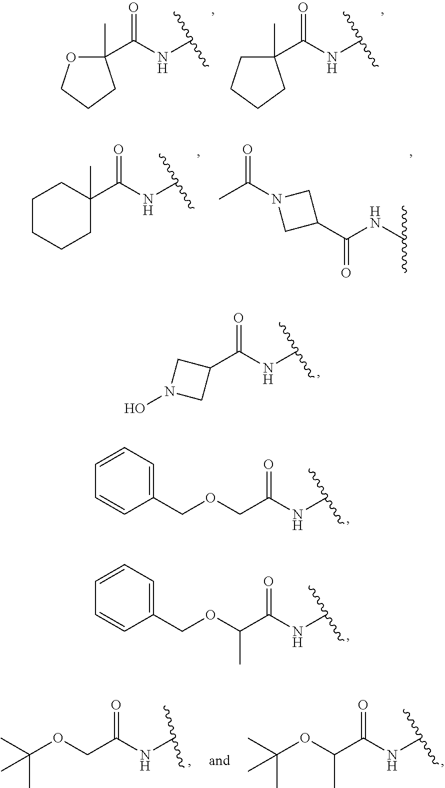

and
R^{C} is selected from:

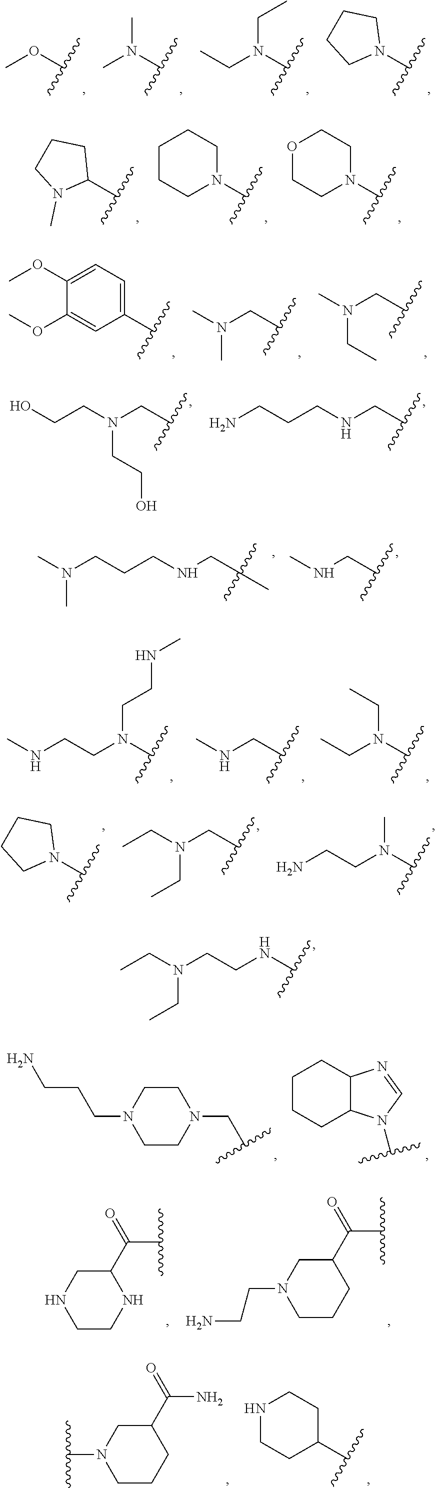

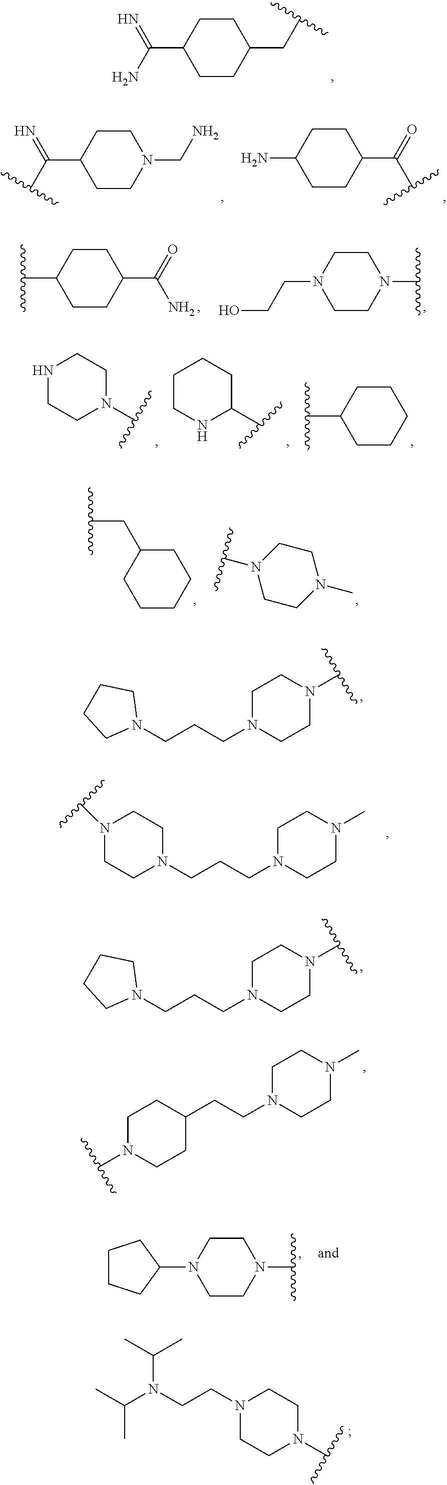

with the proviso that the ionizable lipid is not:

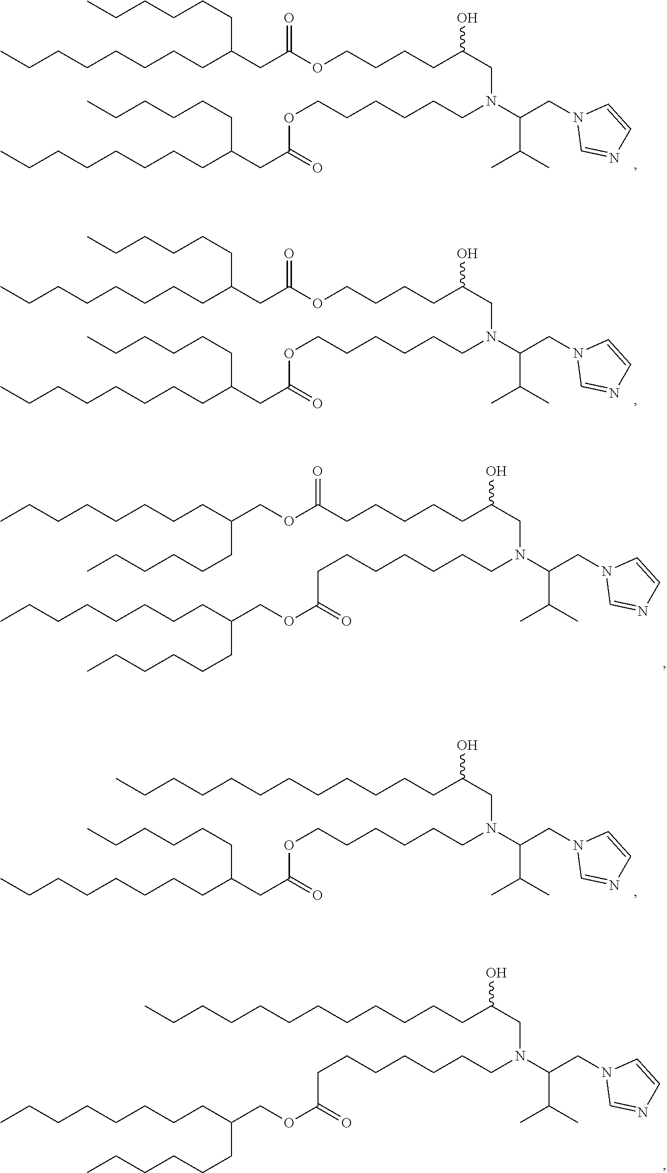 [SMILES: CCCCCCCCC(CCCCCC)CC(=O)OCCCCCCN(CC(O)CCCCOC(=O)CC(CCCCCC)CCCCCCCC)C(Cn1ccnc1)C(C)C ; CCCCCCCCC(CCCCCC)CC(=O)OCCCCCCN(CC(O)CCCCOC(=O)CC(CCCCCC)CCCCCCCC)C(Cn1ccnc1)C(C)C ; CCCCCCCCC(CCCCCC)COC(=O)CCCCCCCN(CC(O)CCCCCC(=O)OCC(CCCCCC)CCCCCCCC)C(Cn1ccnc1)C(C)C ; CCCCCCCCCCCCC(O)CN(CCCCCCOC(=O)CC(CCCCCC)CCCCCCCC)C(Cn1ccnc1)C(C)C ; CCCCCCCCCCCCC(O)CN(CCCCCCCC(=O)OCC(CCCCCC)CCCCCCCC)C(Cn1ccnc1)C(C)C]

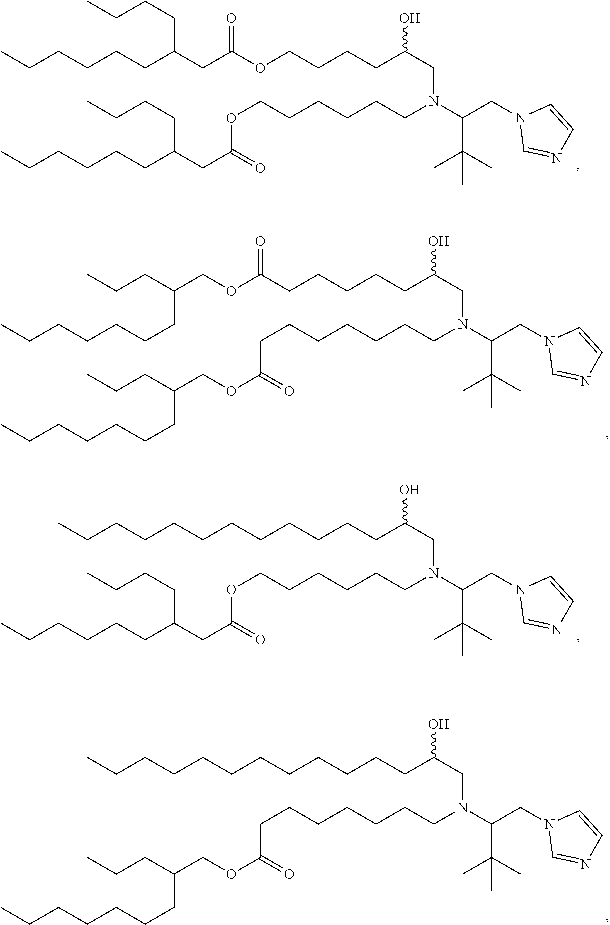 [SMILES: CCCCCCC(CCCC)CC(=O)OCCCCCCN(CC(O)CCCCOC(=O)CC(CCCC)CCCCCC)C(Cn1ccnc1)C(C)(C)C ; CCCCCCCC(CCC)COC(=O)CCCCCCCN(CC(O)CCCCCC(=O)OCC(CCC)CCCCCCC)C(Cn1ccnc1)C(C)(C)C ; CCCCCCCCCCCCC(O)CN(CCCCCCOC(=O)CC(CCCC)CCCCCC)C(Cn1ccnc1)C(C)(C)C ; CCCCCCCCCCCCC(O)CN(CCCCCCCC(=O)OCC(CCC)CCCCCCC)C(Cn1ccnc1)C(C)(C)C]

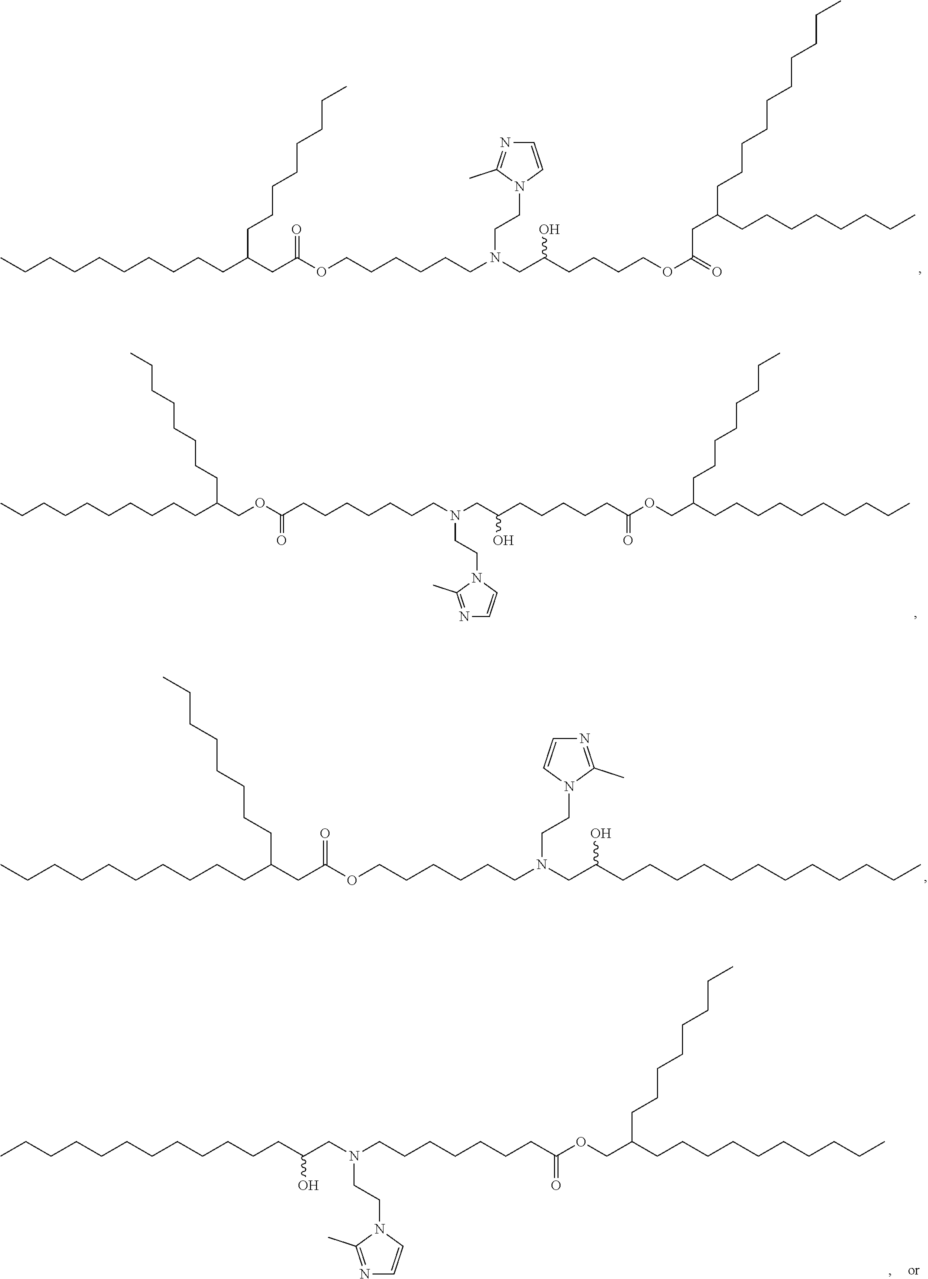

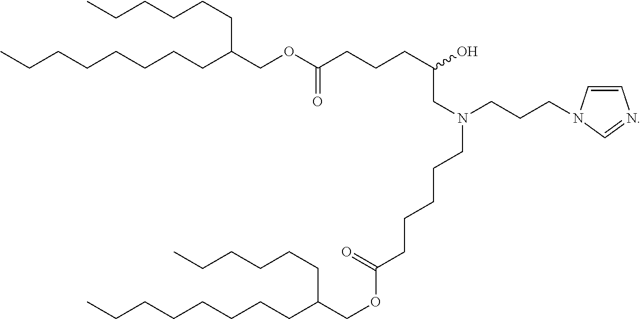 [SMILES: CCCCCCCCC(CCCCCC)COC(=O)CCCCCN(CCCn1ccnc1)CC(O)CCCC(=O)OCC(CCCCCC)CCCCCCCC]

In some embodiments of Formula (22), R³ is selected from:

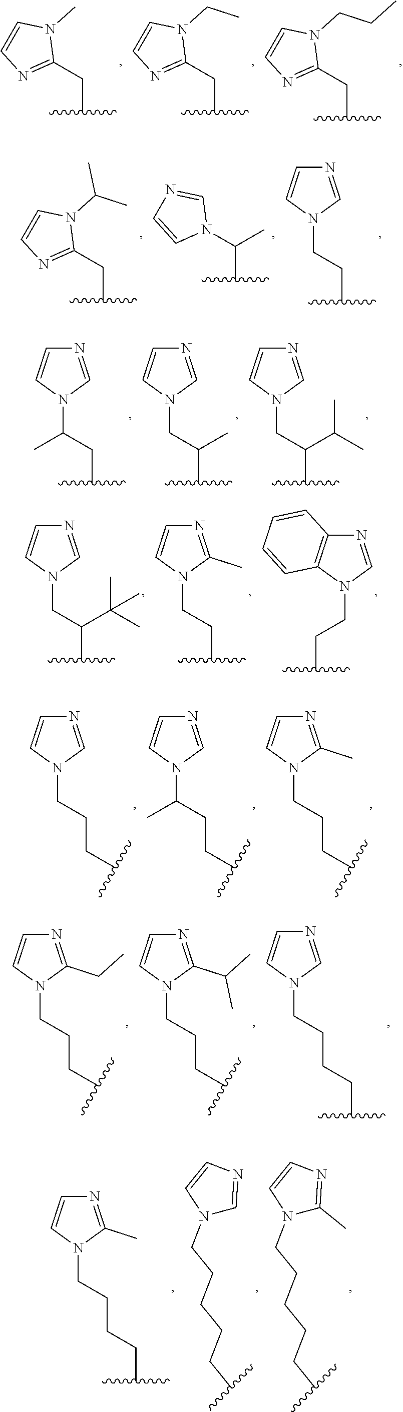

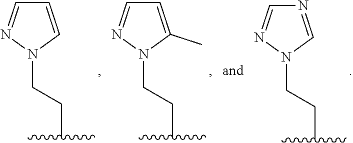

In some embodiments of Formula (22), R¹ is R¹*, R² is R²*, and the ionizable lipid is of Formula (23):

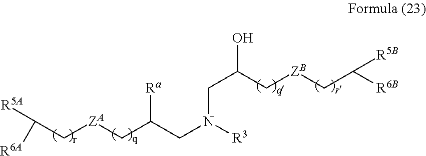

wherein:
q and q′ are each independently integers from 0 to 12;
r and r′ are each independently integers from 0 to 6, wherein at least one of r or r′ is not 0;
Z^{A} and Z^{B} are each independently selected from —C(O)O—, \hat{ }—OC(O), and —OC(O)O—; where \hat{ } denotes the attachment point to —(CH₂)_{q}— or —(CH₂)_{q′}—; and
R^{5A}, R^{5B}, R^{6A}, and R^{6B} are each independently C₁-C₂₀ alkyl or C₂-C₂₀ alkenyl.

In some embodiments of Formula (23), Z^{A} and Z^{B} are \hat{ }—C(O)O—, and the ionizable lipid is of Formula (23a-1):

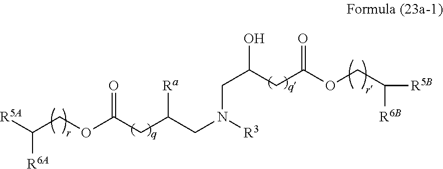

In some embodiments of Formula (23), Z^{A} and Z^{B} are \hat{ }—OC(O)—, and the ionizable lipid is of Formula (23a-2)

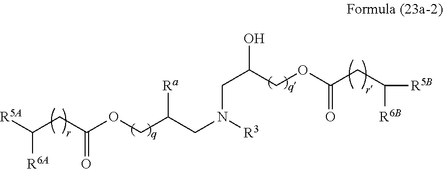

In some embodiments of Formula (23), Z^{A} and Z^{B} are —O(C)(O)O—, and the ionizable lipid is represented by Formula (23a-3):

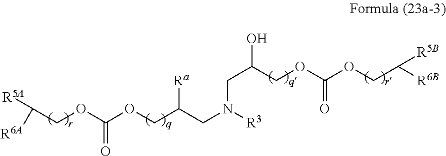

In some embodiments of Formula (22), R² is C₁-C₃₀ alkyl, and the ionizable lipid is of Formula (25):

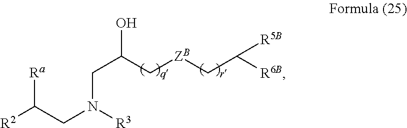

or is a pharmaceutically acceptable salt thereof, wherein:
Z^{B} is selected from \hat{ }—C(O)O—, \hat{ }—OC(O)—, and —OC(O)O—; where \hat{ } denotes the attachment point to —(CH₂)_{q′}—;
R^{5B} and R^{6B} are each independently C₁-C₂₀ alkyl or C₂-C₂₀ alkenyl;
q′ is an integer from 0 to 12; and
r′ is an integer from 1 to 6.

In some embodiments of Formula (25), Z^{B} is \hat{ }—C(O)O—, and the ionizable lipid is of Formula (25a-1):

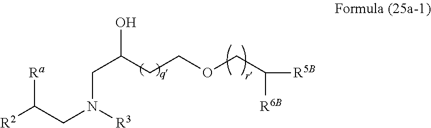

In some embodiments of Formula (25), Z^{B} is \hat{ }—OC(O)—, and the ionizable lipid is of Formula (25a-2):

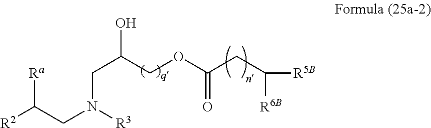

In some embodiments of Formula (25), Z^{B} is —OC(O)O—, and the ionizable lipid is of Formula (25a-3):

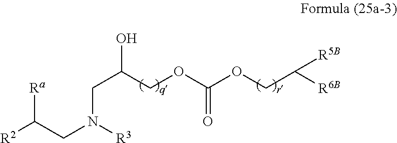

In some embodiments, an ionizable lipid is selected from the table below:

| Lipid | Structure |
|---|---|
| 100-a | 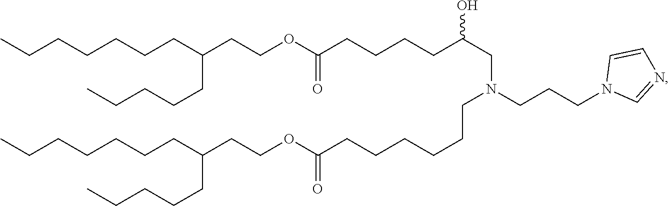 [SMILES: CCCCCCCC(CCCCC)CCOC(=O)CCCCCCN(CCCn1ccnc1)CC(O)CCCCC(=O)OCCC(CCCCC)CCCCCCC] |
| 100-b | 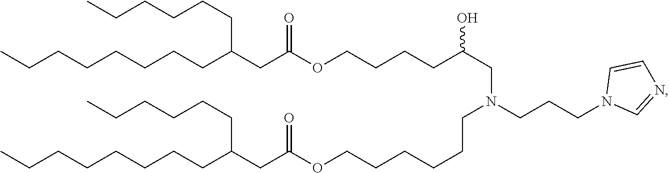 [SMILES: CCCCCCCCC(CCCCCC)CC(=O)OCCCCCCN(CCCn1ccnc1)CC(O)CCCCOC(=O)CC(CCCCCC)CCCCCCCC] |
| 100-c | 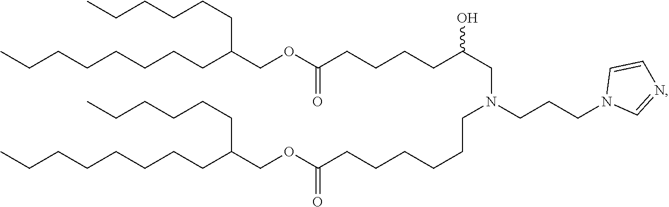 [SMILES: CCCCCCCCC(CCCCCC)COC(=O)CCCCCCN(CCCn1ccnc1)CC(O)CCCCC(=O)OCC(CCCCCC)CCCCCCCC] |
| 100-d | 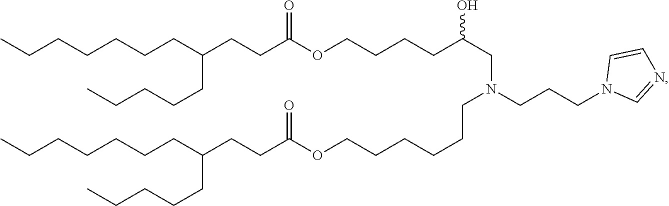 [SMILES: CCCCCCCC(CCCCC)CCC(=O)OCCCCCCN(CCCn1ccnc1)CC(O)CCCCOC(=O)CCC(CCCCC)CCCCCCC] |
| 100-e | 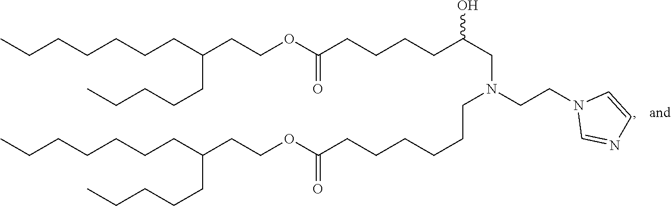 [SMILES: CCCCCCCC(CCCCC)CCOC(=O)CCCCCCN(CCn1ccnc1)CC(O)CCCCC(=O)OCCC(CCCCC)CCCCCCC] |
| | and |
| 100-f | 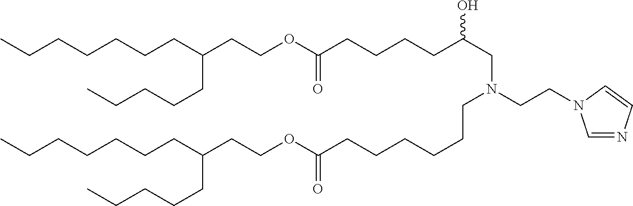 [SMILES: CCCCCCCC(CCCCC)CCOC(=O)CCCCCCN(CCn1ccnc1)CC(O)CCCCC(=O)OCCC(CCCCC)CCCCCCC] |

In some embodiments, an ionizable lipid is selected from the table below.

| Lipid | Structure |
|---|---|
| 1-al | 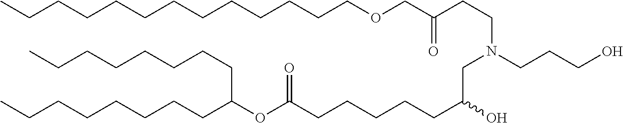 [SMILES: CCCCCCCCCCCCCOCC(=O)CCN(CCCO)CC(O)CCCCCC(=O)OC(CCCCCCCC)CCCCCCCC] |
| 1-am | 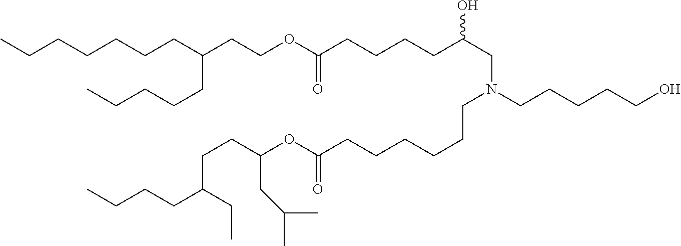 [SMILES: CCCCCCCC(CCCCC)CCOC(=O)CCCCC(O)CN(CCCCCO)CCCCCCC(=O)OC(CCC(CC)CCCC)CC(C)C] |
| 1-an | 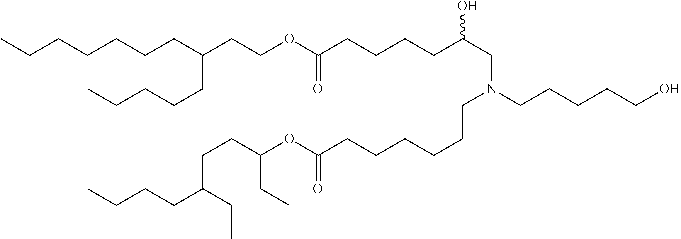 [SMILES: CCCCCCCC(CCCCC)CCOC(=O)CCCCC(O)CN(CCCCCO)CCCCCCC(=O)OC(CC)CCC(CC)CCCC] |
| 1-ao | 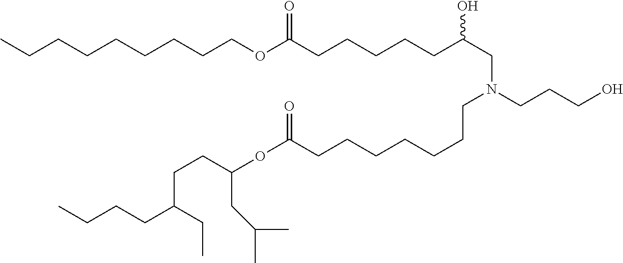 [SMILES: CCCCCCCCCOC(=O)CCCCCC(O)CN(CCCO)CCCCCCCC(=O)OC(CCC(CC)CCCC)CC(C)C] |
| 1-ap | 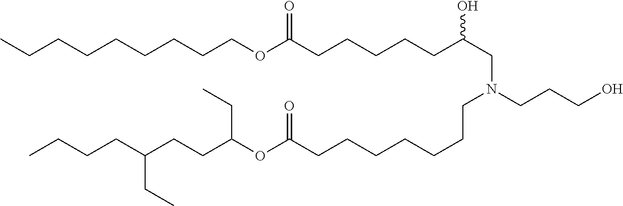 [SMILES: CCCCCCCCCOC(=O)CCCCCC(O)CN(CCCO)CCCCCCCC(=O)OC(CC)CCC(CC)CCCC] |
| 1-aq | 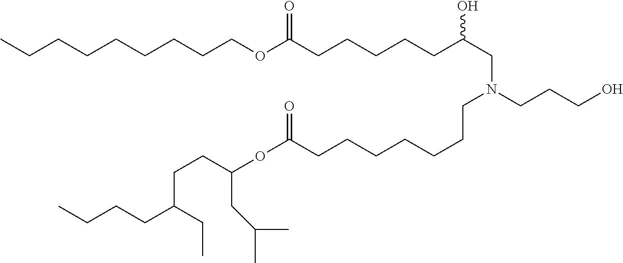 [SMILES: CCCCCCCCCOC(=O)CCCCCC(O)CN(CCCO)CCCCCCCC(=O)OC(CCC(CC)CCCC)CC(C)C] |
| 1-ar | 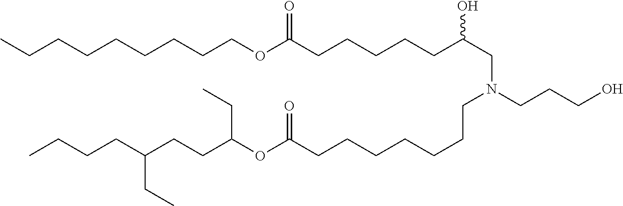 [SMILES: CCCCCCCCCOC(=O)CCCCCC(O)CN(CCCO)CCCCCCCC(=O)OC(CC)CCC(CC)CCCC] |
| 1-as | 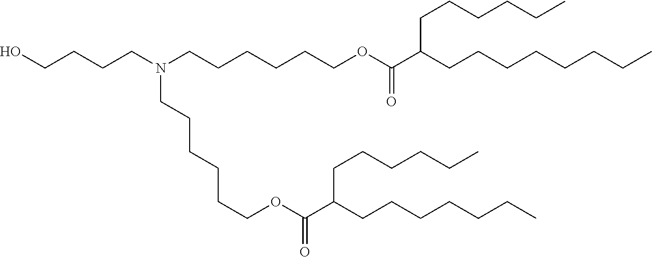 [SMILES: CCCCCCCCC(CCCCCC)C(=O)OCCCCCCN(CCCCO)CCCCCCOC(=O)C(CCCCCC)CCCCCCC] |
| 1-at | 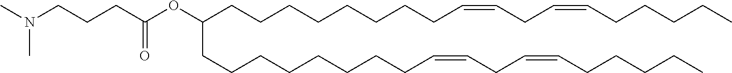 [SMILES: CCCCC/C=C\C/C=C\CCCCCCCCC(CCCCCCCC/C=C\C/C=C\CCCCC)OC(=O)CCCN(C)C] |
| 1-au | 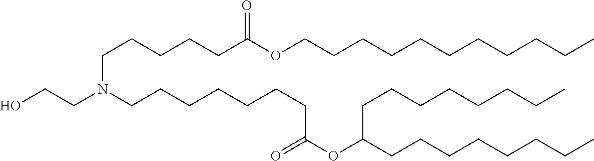 [SMILES: CCCCCCCCCCCOC(=O)CCCCCN(CCO)CCCCCCCC(=O)OC(CCCCCCCC)CCCCCCCC] |

In some embodiments, an ionizable lipid is described in US patent publication number 20190321489. In some embodiments, an ionizable lipid is described in international patent publication WO 2010/053572, incorporated herein by reference. In some embodiments, an ionizable lipid is C12-200, described at paragraph [00225] of WO 2010/053572.

Several ionizable lipids have been described in the literature, many of which are commercially available. In certain embodiments, such ionizable lipids are included in the transfer vehicles described herein. In some embodiments, the ionizable lipid N-[1-(2,3-dioleyloxy)propyl]-N,N,N-trimethylammonium chloride or “DOTMA” is used. (Felgner et al. Proc. Nat'l Acad. Sci. 84, 7413 (1987); U.S. Pat. No. 4,897,355). DOTMA can be formulated alone or can be combined with a neutral lipid, dioleoylphosphatidylethanolamine or “DOPE” or other cationic or non-cationic lipids into a lipid nanoparticle. Other suitable cationic lipids include, for example, ionizable cationic lipids as described in U.S. provisional patent application 61/617,468, filed Mar. 29, 2012 (incorporated herein by reference), such as, e.g., (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), and (15Z,18Z)—N,N-dimethyl-6-((9Z,12Z)-octadeca-9,12-dien-1-yl)tetracosa-5,15,18-trien-1-amine (HGT5002), C12-200 (described in WO 2010/053572), 2-(2,2-di((9Z,12Z)-octadeca-9,12-dien-1-yl)-1,3-dioxolan-4-yl)-N,N-dimethylethanamine (DLinKC2-DMA)) (See, WO 2010/042877; Semple et al., Nature Biotech. 28:172-176 (2010)), 2-(2,2-di((9Z,2Z)-octadeca-9,12-dien-1-yl)-1,3-dioxolan-4-yl)-N,N-dimethylethanamine (DLin-KC2-DMA), (3S,10R,13R,17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), (15Z,18Z)—N,N-dimethyl-6-(9Z,12Z)-octadeca-9,12-dien-1-yl)tetracosa-15,18-dien-1-amine (HGT5000), (15Z,18Z)—N,N-dimethyl-6-((9Z,12Z)-octadeca-9,12-dien-1-yl)tetracosa-4,15,18-trien-1-amine (HGT5001), (15Z,18 Z)—N,N-dimethyl-6-((9Z,12Z)-octadeca-9,12-dien-1-yl)tetracosa-5,15,18-trien-1-amine (HGT5002), 5-carboxyspermylglycine-dioctadecylamide (DOGS), 2,3-dioleyloxy-N-[2(spermine-carboxamido)ethyl]-N,N-dimethyl-1-propanaminium (DOSPA) (Behr et al. Proc. Nat.'l Acad. Sci. 86, 6982 (1989); U.S. Pat. Nos. 5,171,678; 5,334,761), 1,2-Dioleoyl-3-Dimethylammonium-Propane (DODAP), 1,2-Dioleoyl-3-Trimethylammonium-Propane or (DOTAP). Contemplated ionizable lipids also include 1,2-distcaryloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′, 1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylamninopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA) or GL67, or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1). The use of cholesterol-based ionizable lipids to formulate the transfer vehicles (e.g., lipid nanoparticles) is also contemplated by the present invention. Such cholesterol-based ionizable lipids can be used, either alone or in combination with other lipids. Suitable cholesterol-based ionizable lipids include, for example, DC-Cholesterol (N,N-dimethyl-N-ethylcarboxamidocholesterol), and 1,4-bis(3-N-oleylamino-propyl)piperazine (Gao, et al., Biochem. Biophys. Res. Comm. 179, 280 (1991); Wolf et al. BioTechniques 23, 139 (1997); U.S. Pat. No. 5,744,335).

Also contemplated are cationic lipids such as dialkylamino-based, imidazole-based, and guanidinium-based lipids. For example, also contemplated is the use of the ionizable lipid (3S,10R, 13R, 17R)-10,13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate (ICE), as disclosed in International Application No. PCT/US2010/058457, incorporated herein by reference.

Also contemplated are ionizable lipids such as the dialkylamino-based, imidazole-based, and guanidinium-based lipids. For example, certain embodiments are directed to a composition comprising one or more imidazole-based ionizable lipids.

Without wishing to be bound by a particular theory, it is believed that the fusogenicity of the imidazole-based cationic lipid is related to the endosomal disruption which is facilitated by the imidazole group, which has a lower pKa relative to traditional ionizable lipids. The endosomal disruption in turn promotes osmotic swelling and the disruption of the liposomal membrane, followed by the transfection or intracellular release of the nucleic acid(s) contents loaded therein into the target cell.

The imidazole-based ionizable lipids are also characterized by their reduced toxicity relative to other ionizable lipids.

a. Amine Lipids

In certain embodiments, transfer vehicle compositions for the delivery of circular RNA comprise an amine lipid. In certain embodiments, an ionizable lipid is an amine lipid. In some embodiments, an amine lipid is described in international patent application PCT/US2018/053569.

In some embodiments, the amine lipid is Lipid E, which is (9Z, 12Z)-3-((4,4-bis(octyloxy)butanoyl)oxy)-2-((((3-(diethylamino)propoxy)carbonyl)oxy)methyl)propyl octadeca-9, 12-dienoate.

Lipid E can be depicted as:

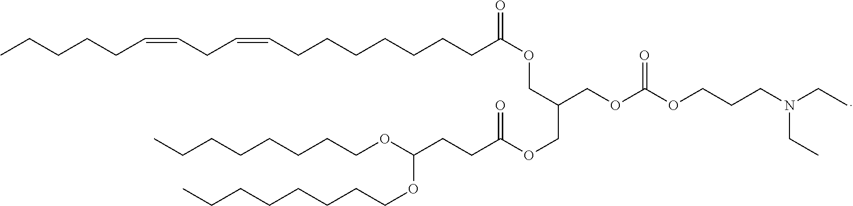 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid E may be synthesized according to WO2015/095340 (e.g., pp. 84-86). In certain embodiments, the amine lipid is an equivalent to Lipid E.

In certain embodiments, an amine lipid is an analog of Lipid E. In certain embodiments, a Lipid E analog is an acetal analog of Lipid E. In particular transfer vehicle compositions, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11 and C12 acetal analog.

Amine lipids and other biodegradable lipids suitable for use in the transfer vehicles, e.g., lipid nanoparticles, described herein are biodegradable in vivo. The amine lipids described herein have low toxicity (e.g., are tolerated in animal models without adverse effect in amounts of greater than or equal to 10 mg/kg). In certain embodiments, transfer vehicles composing an amine lipid include those where at least 75% of the amine lipid is cleared from the plasma within 8, 10, 12, 24, or 48 hours, or 3, 4, 5, 6, 7, or 10 days.

Biodegradable lipids include, for example, the biodegradable lipids of WO2017/173054, WO2015/095340, and WO2014/136086.

Lipid clearance may be measured by methods known by persons of skill in the art. See, for example, Maier, M. A., et al. Biodegradable Lipids Enabling Rapidly Eliminated Lipid Nanoparticles for Systemic Delivery of RNAi Therapeutics. Mol. Ther. 2013, 21(8), 1570-78.

Transfer vehicle compositions comprising an amine lipid can lead to an increased clearance rate. In some embodiments, the clearance rate is a lipid clearance rate, for example the rate at which a lipid is cleared from the blood, serum, or plasma. In some embodiments, the clearance rate is an RNA clearance rate, for example the rate at which an circRNA is cleared from the blood, serum, or plasma. In some embodiments, the clearance rate is the rate at which transfer vehicles are cleared from the blood, serum, or plasma. In some embodiments, the clearance rate is the rate at which transfer vehicles are cleared from a tissue, such as liver tissue or spleen tissue. In certain embodiments, a high rate of clearance leads to a safety profile with no substantial adverse effects. The amine lipids and biodegradable lipids may reduce transfer vehicle accumulation in circulation and in tissues. In some embodiments, a reduction in transfer vehicle accumulation in circulation and in tissues leads to a safety profile with no substantial adverse effects.

Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood, where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g., to the liver. Further, it has been found that lipids with a pKa ranging from about 5.3 to about 6.4 are effective for delivery in vivo, e.g., into tumors. See, e.g., WO2014/136086.

b. Lipids Containing a Disulfide Bond

A lipid of the present invention may have an S—S(disulfide) bond. These lipid-like compounds are useful for delivery of pharmaceutical agents. They can be preliminarily screened for their efficacy in delivering pharmaceutical agents by an in vitro assay and then confirmed by animal experiments and clinic trials. Other methods will also be apparent to those of ordinary skill in the art.

The above described complexes can be prepared using procedures described in publications such as Wang et al., ACS Synthetic Biology, 1, 403-07 (2012). Generally, they are obtained by incubating a lipid-like compound and a pharmaceutical agent in a buffer such as a sodium acetate buffer or a phosphate buffered saline (“PBS”).

i. Hydrophilic Groups

In certain embodiments, the selected hydrophilic functional group or moiety may alter or otherwise impart properties to the compound or to the transfer vehicle of which such compound is a component (e.g., by improving the transfection efficiencies of a lipid nanoparticle of which the compound is a component). For example, the incorporation of guanidinium as a hydrophilic head-group in the compounds disclosed herein may promote the fusogenicity of such compounds (or of the transfer vehicle of which such compounds are a component) with the cell membrane of one or more target cells, thereby enhancing, for example, the transfection efficiencies of such compounds. It has been hypothesized that the nitrogen from the hydrophilic guanidinium moiety forms a six-membered ring transition state which grants stability to the interaction and thus allows for cellular uptake of encapsulated materials. (Wender, et al., Adv. Drug Del. Rev. (2008) 60: 452-472.) Similarly, the incorporation of one or more amino groups or moieties into the disclosed compounds (e.g., as a head-group) may further promote disruption of the endosomal/lysosomal membrane of the target cell by exploiting the fusogenicity of such amino groups. This is based not only on the pKa of the amino group of the composition, but also on the ability of the amino group to undergo a hexagonal phase transition and fuse with the target cell surface, i.e. the vesicle membrane. (Koltover, et al. Science (1998) 281: 78-81.) The result is believed to promote the disruption of the vesicle membrane and release of the lipid nanoparticle contents into the target cell.

Similarly, in certain embodiments the incorporation of, for example, imidazole as a hydrophilic head-group in the compounds disclosed herein may serve to promote endosomal or lysosomal release of, for example, contents that are encapsulated in a transfer vehicle (e.g., lipid nanoparticle) of the invention. Such enhanced release may be achieved by one or both of a proton-sponge mediated disruption mechanism and/or an enhanced fusogenicity mechanism. The proton-sponge mechanism is based on the ability of a compound, and in particular a functional moiety or group of the compound, to buffer the acidification of the endosome. This may be manipulated or otherwise controlled by the pKa of the compound or of one or more of the functional groups comprising such compound (e.g., imidazole). Accordingly, in certain embodiments the fusogenicity of, for example, the imidazole-based compounds disclosed herein (e.g., HGT4001 and HGT4004) are related to the endosomal disruption properties, which are facilitated by such imidazole groups, which have a lower pKa relative to other traditional ionizable lipids. Such endosomal disruption properties in turn promote osmotic swelling and the disruption of the liposomal membrane, followed by the transfection or intracellular release of the polynucleotide materials loaded or encapsulated therein into the target cell. This phenomenon can be applicable to a variety of compounds with desirable pKa profiles in addition to an imidazole moiety. Such embodiments also include multi-nitrogen based functionalities such as polyamines, polypeptide (histidine), and nitrogen-based dendritic structures.

### B. Stabilizing Lipids

A stabilizing lipid or surface stabilizing lipid may be used to enhance the structure of the LNP. A stabilizing lipid as contemplated herein may be a polyethylene glycol (PEG)-modified phospholipid. The use and inclusion of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C8 PEG-2000 ceramide) in the liposomal and pharmaceutical compositions described herein is contemplated, preferably in combination with one or more of the compounds and lipids disclosed herein. Contemplated PEG-modified lipids include, but are not limited to, a polyethylene glycol chain of up to 5 kDa in length covalently attached to a lipid with alkyl chain(s) of C6-C20 length. In some embodiments, the PEG-modified lipid employed in the compositions and methods of the invention is 1,2-dimyristoyl-sn-glycerol, methoxypolyethylene Glycol (2000 MW PEG) “DMG-PEG2000.” The addition of PEG-modified lipids to the lipid delivery vehicle may prevent complex aggregation and may also provide a means for increasing circulation lifetime and increasing the delivery of the lipid-polynucleotide composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in a liposomal lipid nanoparticle.

In an embodiment, a PEG-modified lipid is described in International Pat. Appl. No. PCT/US2019/015913, which is incorporated herein by reference in their entirety. In an embodiment, a transfer vehicle comprises one or more PEG-modified lipids.

Non-limiting examples of PEG-modified lipids include PEG-modified phosphatidylethanolamines and phosphatidic acids, PEG-ceramide conjugates (e.g., PEG-CerC14 or PEG-CerC20), PEG-modified dialkylamines and PEG-modified 1,2-diacyloxypropan-3-amines. In some further enbodiments, a PEG-modified lipid may be, e,g, PEG-c-DOMG, PEG-DMG, PEG-DLPE, PEG-DMPE, PEG-DPPC, or a PEG-DSPE.

In some still further embodiments, the PEG-modified lipid includes, but is not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In various embodiments, a PEG-modified lipid may also be referred to as “PEGylated lipid” or “PEG-lipid.”

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C14 to about C22, such as from about C14 to about C16. In some embodiments, a PEG moiety, for example a mPEG-NH₂, has a size of about 1000, about 2000, about 5000, about 10,000, about 15,000 or about 20,000 Daltons. In one embodiment, the PEG-lipid is PEG2k-DMG.

In one embodiment, the lipid nanoparticles described herein can comprise a lipid modified with a non-diffusible PEG. Non-limiting examples of non-diffusible PEGs include PEG-DSG and PEG-DSPE.

PEG-lipids are known in the art, such as those described in U.S. Pat. No. 8,158,601 and International Pat. Publ. No. WO2015/130584 A2, which are incorporated herein by reference in their entirety.

In various embodiments, lipids (e.g., PEG-lipids), described herein may be synthesized as described International Pat. Publ. No. PCT/US2016/000129, which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

### C. Helper Lipids

In some embodiments, the transfer vehicle (e.g., LNP) described herein comprises one or more non-cationic helper lipids. In some embodiments, the helper lipid is a phospholipid. In some embodiments, the helper lipid is a phospholipid substitute or replacement. In some embodiments, the phospholipid or phospholipid substitute can be, for example, one or more saturated or (poly)unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the helper lipid is a 1,2-distearoyl-177-glycero-3-phosphocholine (DSPC) analog, a DSPC substitute, oleic acid, or an oleic acid analog.

In some embodiments, a helper lipid is a non-phosphatidyl choline (PC) zwitterionic lipid, a DSPC analog, oleic acid, an oleic acid analog, or a DSPC substitute.

In some embodiments, a helper lipid is described in PCT/US2018/053569. Helper lipids suitable for use in a lipid composition of the disclosure include, for example, a variety of neutral, uncharged or zwitterionic lipids. Such helper lipids are preferably used in combination with one or more of the compounds and lipids disclosed herein. Examples of helper lipids include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoylsn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-paimitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), paimitoyioieoyl phosphatidylcholine (POPC), lysophosphatidyl choline, dioleoyl phosphatidylethanol amine (DOPE) dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the helper lipid may be distearoylphosphatidylcholine (DSPC) or dimyristoyl phosphatidyl ethanolamine (DMPE). In another embodiment, the helper lipid may be distearoylphosphatidylcholine (DSPC). Helper lipids function to stabilize and improve processing of the transfer vehicles. Such helper lipids are preferably used in combination with other excipients, for example, one or more of the ionizable lipids disclosed herein. In some embodiments, when used in combination with an ionizable lipid, the helper lipid may comprise a molar ratio of 5% to about 90%, or about 10% to about 70% of the total lipid present in the lipid nanoparticle.

### D. Structural Lipids

The transfer vehicles described herein comprise one or more structural lipids. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

The transfer vehicles described herein comprise one or more structural lipids. Incorporation of structural lipids in a transfer vehicle, e.g., a lipid nanoparticle, may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, or alpha-tocopherol.

In some embodiments, a transfer vehicle includes an effective amount of an immune cell delivery potentiating lipid, e.g., a cholesterol analog or an amino lipid or combination thereof, that, when present in a transfer vehicle, e.g., an lipid nanoparticle, may function by enhancing cellular association and/or uptake, internalization, intracellular trafficking and/or processing, and/or endosomal escape and/or may enhance recognition by and/or binding to immune cells, relative to a transfer vehicle lacking the immune cell delivery potentiating lipid. Accordingly, while not intending to be bound by any particular mechanism or theory, in one embodiment, a structural lipid or other immune cell delivery potentiating lipid of the disclosure binds to C1q or promotes the binding of a transfer vehicle comprising such lipid to C1q. Thus, for in vitro use of the transfer vehicles of the disclosure for delivery of a nucleic acid molecule to an immune cell, culture conditions that include C1q are used (e.g., use of culture media that includes serum or addition of exogenous C1q to serum-free media). For in vivo use of the transfer vehicles of the disclosure, the requirement for C1q is supplied by endogenous C1q.

In certain embodiments, the structural lipid is cholesterol. In certain embodiments, the structural lipid is an analog of cholesterol.

### E. Lipid Nanoparticle (LNP) Formulations

In various embodiments, lipid nanoparticles (LNPs) may be used as a transfer vehicle to deliver a disclosed circular RNA (e.g., to one or more target cells).

The formation of a lipid nanoparticle (LNP) described herein may be accomplished by any methods known in the art. For example, as described in U.S. Pat. Pub. No. US2012/0178702 A1, which is incorporated herein by reference in its entirety. Non-limiting examples of lipid nanoparticle compositions and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578 (the contents of each of which are incorporated herein by reference in their entirety).

In one embodiment, the LNP formulation may be prepared by, e.g., the methods described in International Pat. Pub. No. WO 2011/127255 or WO 2008/103276, the contents of each of which are herein incorporated by reference in their entirety.

In one embodiment, LNP formulations described herein may comprise a polycationic composition. As a non-limiting example, the polycationic composition may be a composition selected from Formulae 1-60 of U.S. Pat. Pub. No. US2005/0222064 A1, the content of which is herein incorporated by reference in its entirety.

In one embodiment, the lipid nanoparticle may be formulated by the methods described in U.S. Pat. Pub. No. US2013/0156845 A1, and International Pat. Pub. No. WO2013/093648 A2 or WO2012/024526 A2, each of which is herein incorporated by reference in its entirety.

In one embodiment, the lipid nanoparticles described herein may be made in a sterile environment by the system and/or methods described in U.S. Pat. Pub. No. US2013/0164400 A1, which is incorporated herein by reference in its entirety.

In one embodiment, the LNP formulation may be formulated in a nanoparticle such as a nucleic acid-lipid particle described in U.S. Pat. No. 8,492,359, which is incorporated herein by reference in its entirety.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

In some embodiments, the lipid nanoparticles described herein may be synthesized using methods comprising microfluidic mixers. Exemplary microfluidic mixers may include, but are not limited to, a slit interdigital micromixer including, but not limited to, those manufactured by Precision Nanosystems (Vancouver, BC, Canada), Microinnova (Allerheiligen bei Wildon, Austria) and/or a staggered herringbone micromixer (SHM) (Zhigaltsev, I. V. et al. (2012) Langmuir. 28:3633-40; Belliveau, N. M. et al. Mol. Ther. Nucleic. Acids. (2012) 1:e37; Chen, D. et al. J. Am. Chem. Soc. (2012) 134(16):6948-51; each of which is herein incorporated by reference in its entirety).

In some embodiments, methods of LNP generation comprising SHM, further comprise the mixing of at least two input streams wherein mixing occurs by microstructure-induced chaotic advection (MICA). According to this method, fluid streams flow through channels present in a herringbone pattern causing rotational flow and folding the fluids around each other. This method may also comprise a surface for fluid mixing wherein the surface changes orientations during fluid cycling. Methods of generating LNPs using SHM include those disclosed in U.S. Pat. Pub. Nos. US2004/0262223 A1 and US2012/0276209 A1, each of which is incorporated herein by reference in their entirety.

In one embodiment, the lipid nanoparticles may be formulated using a micromixer such as, but not limited to, a Slit Interdigital Microstructured Mixer (SIMM-V2) or a Standard Slit Interdigital Micro Mixer (SSIMM) or Caterpillar (CPMM) or Impinging-jet (IJMM) from the Institut fur Mikrotechnik Mainz GmbH, Mainz Germany). In one embodiment, the lipid nanoparticles are created using microfluidic technology (see, Whitesides (2006) Nature. 442: 368-373; and Abraham et al. (2002) Science. 295: 647-651; each of which is herein incorporated by reference in its entirety). As a non-limiting example, controlled microfluidic formulation includes a passive method for mixing streams of steady pressure-driven flows in micro channels at a low Reynolds number (see, e.g., Abraham et al. (2002) Science. 295: 647651; which is herein incorporated by reference in its entirety).

In one embodiment, the circRNA of the present invention may be formulated in lipid nanoparticles created using a micromixer chip such as, but not limited to, those from Harvard Apparatus (Holliston, MA), Dolomite Microfluidics (Royston, UK), or Precision Nanosystems (Van Couver, BC, Canada). A micromixer chip can be used for rapid mixing of two or more fluid streams with a split and recombine mechanism.

In some embodiments, the LNP of the present disclosure comprises a molar ratio of between about 40% and about 60% ionizable lipid, a molar ratio of between about 3.5% and about 14% helper lipid, a molar ratio of between about 28% and about 50% structural lipid, and a molar ratio of between about 0.5% and about 5% PEG-lipid, inclusive of all endpoints. In some embodiments, the total molar percentage of the ionizable lipid, the helper lipid, the structural lipid, and the PEG-lipid is 100% in the LNP.

In some embodiments, the molar ratio of the ionizable lipid in the LNP is from about 40 to about 60% of the total lipid present in the LNP. In some embodiments, the molar ratio of the ionizable lipid in the LNP is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% of the total lipid present in the LNP. In some embodiments, the ionizable lipid is represented by Formula (7). In some embodiments, the ionizable lipid is represented by Formula (8). All values are inclusive of all endpoints.

In some embodiments, the molar ratio of the helper lipid in the LNP is from about 3.5% to about 14% of the total lipid present in the LNP. In some embodiments, the molar ratio of the helper lipid in the LNP is about 3, about 4%, about 5%, about 6%, about 7%, about 8%, about 9%, about 10%, about 11%, about 12%, about 13%, or about 14% of the total lipid present in the LNP. In some embodiments, the helper lipid is DSPC. In some embodiments, the helper lipid is DOPE. All values are inclusive of all endpoints.

In some embodiments, the molar ratio of the structural lipid in the LNP is from about 28% to about 50% of the total lipid present in the LNP. In some embodiments, the molar ratio of the structural lipid in the LNP is about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the total lipid present in the LNP. In some embodiments, the structural lipid is cholesterol. All values are inclusive of all endpoints.

In some embodiments, the molar ratio of the PEG-lipid in the LNP is from about 0.5% to about 5% of the total lipid present in the LNP. In some embodiments, the molar ratio of the PEG-lipid in the LNP is about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, 3.4%, about 3.5%, about 4.0%, about 4.5%, or about 5% of the total lipid present in the LNP. In some embodiments, the PEG-lipid is DSPE-PEG(2000). In some embodiments, the PEG-lipid is DMG-PEG(2000). All values are inclusive of all endpoints.

In some embodiments, the molar ratio of ionizable lipid:helper lipid: structural lipid:PEG-lipid in the LNP is about 45:9:44:2. In some embodiments, the molar ratio of ionizable lipid:helper lipid:structural lipid:PEG-lipid in the LNP is about 50:10:38.5:1.5. In some embodiments, the molar ratio of ionizable lipid:helper lipid:structural lipid:PEG-lipid in the LNP is about 41:12:45:2. In some embodiments, the molar ratio of ionizable lipid:helper lipid:structural lipid:PEG-lipid in the LNP is about 62:4:33:1. In some embodiments, the molar ratio of ionizable lipid:helper lipid:structural lipid:PEG-lipid in the LNP is about 53:5:41:1. In some embodiments, the molar ratio of each of the ionizable lipid, helper lipid, structural lipid, and PEG-lipid is within 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% of the stated value.

In one embodiment, the lipid nanoparticles may have a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm. In one embodiment, the lipid nanoparticles may have a diameter from about 10 to 500 nm. In one embodiment, the lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm. Each possibility represents a separate embodiment of the present invention.

In some embodiments, a nanoparticle (e.g., a lipid nanoparticle) has a mean diameter of 10-500 nm, 20-400 nm, 30-300 nm, or 40-200 nm. In some embodiments, a nanoparticle (e.g., a lipid nanoparticle) has a mean diameter of 50-150 nm, 50-200 nm, 80-100 nm, or 80-200 nm.

In some embodiments, the lipid nanoparticles described herein can have a diameter from below 0.1 μm to up to 1 mm such as, but not limited to, less than 0.1 μm, less than 1.0 μm, less than 5 μm, less than 10 μm, less than 15 μm, less than 20 μm, less than 25 μm, less than 30 μm, less than 35 μm, less than 40 μm, less than 50 μm, less than 55 μm, less than 60 μm, less than 65 μm, less than 70 μm, less than 75 μm, less than 80 μm, less than 85 μm, less than 90 μm, less than 95 μm, less than 100 μm, less than 125 μm, less than 150 μm, less than 175 μm, less than 200 μm, less than 225 μm, less than 250 μm, less than 275 μm, less than 300 μm, less than 325 μm, less than 350 μm, less than 375 μm, less than 400 μm, less than 425 μm, less than 450 μm, less than 475 μm, less than 500 μm, less than 525 μm, less than 550 μm, less than 575 μm, less than 600 μm, less than 625 μm, less than 650 μm, less than 675 μm, less than 700 μm, less than 725 μm, less than 750 μm, less than 775 μm, less than 800 μm, less than 825 μm, less than 850 μm, less than 875 μm, less than 900 μm, less than 925 μm, less than 950 μm, less than 975 μm.

In another embodiment, LNPs may have a diameter from about 1 nm to about 100 nm, from about 1 nm to about 10 nm, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 50 nM, from about 20 to about 50 nm, from about 30 to about 50 nm, from about 40 to about 50 nm, from about 20 to about 60 nm, from about 30 to about 60 nm, from about 40 to about 60 nm, from about 20 to about 70 nm, from about 30 to about 70 nm, from about 40 to about 70 nm, from about 50 to about 70 nm, from about 60 to about 70 nm, from about 20 to about 80 nm, from about 30 to about 80 nm, from about 40 to about 80 nm, from about 50 to about 80 nm, from about 60 to about 80 nm, from about 20 to about 90 nm, from about 30 to about 90 nm, from about 40 to about 90 nm, from about 50 to about 90 nm, from about 60 to about 90 nm and/or from about 70 to about 90 nm. Each possibility represents a separate embodiment of the present invention.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.1 1, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20. Each possibility represents a separate embodiment of the present invention.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −20 mV to about +20 mV, from about −20 mV to about +15 mV, from about −20 mV to about +10 mV, from about −20 mV to about +5 mV, from about −20 mV to about 0 mV, from about −20 mV to about −5 mV, from about −20 mV to about −10 mV, from about −20 mV to about −15 mV from about −20 mV to about +20 mV, from about −20 mV to about +15 mV, from about −20 mV to about +10 mV, from about −20 mV to about +5 mV, from about −20 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV. Each possibility represents a separate embodiment of the present invention.

The efficiency of encapsulation of a therapeutic agent describes the amount of therapeutic agent that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic agent in a solution containing the nanoparticle composition before and after breaking up the nanoparticle composition with one or more organic solvents or detergents. Fluorescence may be used to measure the amount of free therapeutic agent (e.g., nucleic acids) in a solution. For the nanoparticle compositions described herein, the encapsulation efficiency of a therapeutic agent may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%. Each possibility represents a separate embodiment of the present invention. In some embodiments, the lipid nanoparticle has a polydispersity value of less than 0.4. In some embodiments, the lipid nanoparticle has a net neutral charge at a neutral pH. In some embodiments, the lipid nanoparticle has a mean diameter of 50-200 nm.

The properties of a lipid nanoparticle formulation may be influenced by factors including, but not limited to, the selection of the cationic lipid component, the degree of cationic lipid saturation, the selection of the non-cationic lipid component, the degree of noncationic lipid saturation, the selection of the structural lipid component, the nature of the PEGylation, ratio of all components and biophysical parameters such as size. As described herein, the purity of a PEG lipid component is also important to an LNP's properties and performance.

### F. Methods for Preparing Lipid Nanoparticles (LNP) Formulations

In one embodiment, a lipid nanoparticle formulation may be prepared by the methods described in International Publication Nos. WO2011127255 or WO2008103276, each of which is herein incorporated by reference in their entirety. In some embodiments, lipid nanoparticle formulations may be as described in International Publication No. WO2019131770, which is herein incorporated by reference in its entirety.

In some embodiments, circular RNA is formulated according to a process described in U.S. patent application Ser. No. 15/809,680. In some embodiments, the present invention provides a process of encapsulating circular RNA in transfer vehicles comprising the steps of forming lipids into pre-formed transfer vehicles (i.e. formed in the absence of RNA) and then combining the pre-formed transfer vehicles with RNA. In some embodiments, the novel formulation process results in an RNA formulation with higher potency (peptide or protein expression) and higher efficacy (improvement of a biologically relevant endpoint) both in vitro and in vivo with potentially better tolerability as compared to the same RNA formulation prepared without the step of preforming the lipid nanoparticles (e.g., combining the lipids directly with the RNA).

For certain cationic lipid nanoparticle formulations of RNA, in order to achieve high encapsulation of RNA, the RNA in buffer (e.g., citrate buffer) has to be heated. In those processes or methods, the heating is required to occur before the formulation process (i.e. heating the separate components) as heating post-formulation (post-formation of nanoparticles) does not increase the encapsulation efficiency of the RNA in the lipid nanoparticles. In contrast, in some embodiments of the novel processes of the present invention, the order of heating of RNA does not appear to affect the RNA encapsulation percentage. In some embodiments, no heating (i.e. maintaining at ambient temperature) of one or more of the solutions comprising the pre-formed lipid nanoparticles, the solution comprising the RNA and the mixed solution comprising the lipid nanoparticle encapsulated RNA is required to occur before or after the formulation process.

RNA may be provided in a solution to be mixed with a lipid solution such that the RNA may be encapsulated in lipid nanoparticles. A suitable RNA solution may be any aqueous solution containing RNA to be encapsulated at various concentrations. For example, a suitable RNA solution may contain an RNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable RNA solution may contain an RNA at a concentration in a range from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml.

Typically, a suitable RNA solution may also contain a buffering agent and/or salt. Generally, buffering agents can include HEPES, Tris, ammonium sulfate, sodium bicarbonate, sodium citrate, sodium acetate, potassium phosphate or sodium phosphate. In some embodiments, suitable concentration of the buffering agent may be in a range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM.

Exemplary salts can include sodium chloride, magnesium chloride, and potassium chloride. In some embodiments, suitable concentration of salts in an RNA solution may be in a range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM.

In some embodiments, a suitable RNA solution may have a pH in a range from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5.

Various methods may be used to prepare an RNA solution suitable for the present invention. In some embodiments, RNA may be directly dissolved in a buffer solution described herein. In some embodiments, an RNA solution may be generated by mixing an RNA stock solution with a buffer solution prior to mixing with a lipid solution for encapsulation. In some embodiments, an RNA solution may be generated by mixing an RNA stock solution with a buffer solution immediately before mixing with a lipid solution for encapsulation.

According to the present invention, a lipid solution contains a mixture of lipids suitable to form transfer vehicles for encapsulation of RNA. In some embodiments, a suitable lipid solution is ethanol based. For example, a suitable lipid solution may contain a mixture of desired lipids dissolved in pure ethanol (i.e. 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. In some embodiments, a suitable lipid solution may contain a mixture of desired lipids at a total concentration in a range from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml.

### G. Other Delivery Vehicles Known in the Art

In certain embodiments, other delivery vehicles that are known in the art may be used to transport the circular RNA (i.e., are transfer vehicles encompassed herein).

In some embodiments, liposomes or other lipid bilayer vesicles may be used as a component or as the whole transfer vehicle to facilitate or enhance the delivery and release of circular RAN to one or more target cells. Liposomes are usually characterized by having an interior space sequestered from an outer medium by a membrane of one or more bilayers forming a microscopic sack or vesicle. Bilayer membranes of liposomes are typically formed by lipids, i.e. amphiphilic molecules of synthetic or natural origin that comprise spatially separated hydrophobic or hydrophilic domains (Lasic, D, and Papahadjopoulos, D., eds. Medical Applications of Liposomes. Elsevier, Amsterdam, 1998).

In certain embodiments, the transfer vehicle for transporting the circular RNA comprises a dendrimer. Use of “dendrimer” describes the architectural motif of the transfer vehicle. In some embodiments, the dendrimer includes but is not limited to containing an interior core and one or more layers (i.e. generations) that extend or attach out from the interior core. In some of the embodiments, the generations may contain one or more branching points and an exterior surface of terminal groups that attach to the outermost generation. The branching points, in certain embodiments, may be mostly monodispersed and contain symmetric branching units built around the interior core. In some embodiments, the interior core. Synthesis of the dendrimer may comprise the divergent method, convergent growth, hypercore and branched monomer growth, double exponential growth, lego chemistry, click chemistry and other methods as available in the art (Mendes L. et al., Molecules. 2017. 22 (9): 1401 further describes these methods).

In certain embodiments, as described herein, the transfer vehicle for the circular RNA construct comprises a polymer nanoparticle. In some embodiments, the polymer nanoparticle includes nanocapsules and nanospheres. Nanocapsules, in some embodiments, are composed of an oily core surrounded by a polymeric shell. In some embodiments, the circular RNA is contained within the core and the polymeric shell controls the release of the circular RNA. On the other hand, nanospheres comprise a continuous polymeric network in which the circular RNA is retained or absorbed onto the surface. In some embodiments, cationic polymers are used to encapsulate the circular RNA due to the favorable electrostatic interaction of the cations to the negatively charged nucleic acids and cell membrane. The polymer nanoparticle may be prepared by various methods. In some embodiments, the polymer nanoparticle may be prepared by nanoprecipitation, emulsion techniques, solvent evaporation, solvent diffusion, reverse salting-out or other methods available in the art.

In certain embodiments, as described herein, the transfer vehicle for the circular RNA construct comprises a polymer-lipid hybrid nanoparticle (LPHNP). In some embodiments, the LPHNP comprises a polymer core enveloped within a lipid bilayer. In some embodiments, the polymer core encapsulates the circular RNA construct. In some embodiments, the LPHNP further comprises an outer lipid bilayer. In certain embodiments this outer lipid bilayer comprises a PEG-lipid, helper lipid, cholesterol or other molecule as known in the art to help with stability in a lipid-based nanoparticle. The lipid bilayer closest to the polymer core mitigates the loss of the entrapped circular RNA during LPHNP formation and protects from degradation of the polymer core by preventing diffusion of water from outside of the transfer vehicle into the polymer core (Mukherjee et al., In J. Nanomedicine. 2019; 14: 1937-1952).

In certain embodiments, the circular RNA can be transported using a peptide-based delivery mechanism. In some embodiments, the peptide-based delivery mechanism comprises a lipoprotein. Based on the size of the drug to be delivered, the lipoprotein may be either a low-density (LDL) or high-density lipoprotein (HDL). As seen in U.S. Pat. No. 8,734,853B2, high-density lipoproteins are capable of transporting a nucleic acid in vivo and in vitro. In particular embodiments, the lipid component includes cholesterol. In more particular embodiments, the lipid component includes a combination of cholesterol and cholesterol oleate.

In certain embodiments, the circular RNA construct can be transported using a carbohydrate carrier or a sugar-nanocapsule. In certain embodiments, the carbohydrate carrier comprises a sugar-decorated nanoparticle, peptide- and saccharide-conjugated dendrimer, nanoparticles based on polysaccharides, and other carbohydrate-based carriers available in the art. As described herein, the incorporation of carbohydrate molecules may be through synthetic means. In some embodiments, the carbohydrate carrier comprises polysaccharides. These polysaccharides may be made from the microbial cell wall of the target cell. For example, carbohydrate carriers comprised of mannan carbohydrates have been shown to successfully deliver mRNA (Son et al., Nano Lett. 2020. 20(3): 1499-1509).

In certain embodiments, as provided herein, the transfer vehicle for the circular RNA is a glyconanoparticle (GlycoNP). As known in the art, glyconanoparticles comprise a core comprising gold, iron oxide, semiconductor nanoparticles or a combination thereof. In some embodiments, the glyconanoparticle is functionalized using carbohydrates. In certain embodiments, the glyconanoparticle comprises a carbon nanotube or graphene. In one embodiment the glyconanoparticle comprises a polysaccharide-based GlycoNP (e.g., chitosan-based GlycoNP). In certain embodiments, the glyconanoparticle is a glycodendrimer.

In certain embodiments, as provided herein, the circular RNA is transferred through use of an exosome, a type of extracellular vesicle. Exosomes naturally are secreted by various types of cells and are used as a transport vesicle for various forms of cargo. During delivery exosomes can contain and protect specific mRNAs, regulatory microRNAs, lipids, and proteins (Luan et al., Acta Pharmacologica Sinica. 2017. 38:754-763). Naturally, exosomes may be 30 nm to 125 nm.

In some embodiments, the exosome may be made in part from an immune cell. As shown in Haney et al, use of immune cell derived exosomes are able to avoid mononuclear phagocytes (J Control Release. 2015. 207:18-30). In some embodiments, the exosome may be a dendritic cell, macrophage, T cell, B cell or derived from another immune cell. As seen in WO/2021/041473A1, various forms of RNAs of varying lengths may be transported through exosome delivery including messenger RNA (mRNA), microRNA (miRNA), long intergenic non-coding RNA (lincRNA), long non-coding RNA (lncRNA), non-coding RNA (ncRNA), non-messenger RNA (nmRNA), small RNA (sRNA), small non-messenger RNA (smnRNA), DNA damage response RNA (DD RNA), extracellular RNA (exRNA), small nuclear RNA (snRNA), small nucleolar RNA (snoRNA), and precursor messenger RNA (pre-mRNA).

In other embodiments, the transfer vehicle may comprise in whole or in part from a fusome. In some embodiments, the fusome is derived from an endoplasmic reticulum of a germline cyst. In certain embodiments, the germline cyst is from a Drosophila ovary.

In certain embodiments, the circular RNA construct may be transported using noncellular and instead be through mechanical delivery mechanisms. In some embodiments, this delivery method includes microneedles, electroporation, continuous pumps and/or gene guns.

In some embodiments, the transfer vehicle of the circular RNA construct is a solution or diluent comprising of a salt or a buffer.

Various circular RNA, circular RNA constructs, compositions comprising circular RNA, precursor RNA, lipids, and related methods are described, for example in WO2019236673, WO2020237227, WO2021113777, WO2021226597, WO2021189059, WO2021236855, WO2022261490, WO2023056033, and WO2023081526, each incorporated by reference in their entireties herein.

### 7. Combinations of Proteins and IRES

In certain embodiments, as provided herein, the payload encoded by the circular RNA polynucleotide may be optimized through use of a specific internal ribosome entry sites (IRES) within the translation initiation element (TIE). In some embodiments, IRES specificity within a circular RNA can significantly enhance expression of specific proteins encoded within the coding element.

### 8. Targeting

### A. Targeting Methods

The present invention also contemplates the discriminatory targeting of target cells and tissues by both passive and active targeting means. The phenomenon of passive targeting exploits the natural distributions patterns of a transfer vehicle in vivo without relying upon the use of additional excipients or means to enhance recognition of the transfer vehicle by target cells. For example, transfer vehicles which are subject to phagocytosis by the cells of the reticulo-endothelial system are likely to accumulate in the liver or spleen, and accordingly may provide a means to passively direct the delivery of the compositions to such target cells.

Alternatively, the present invention contemplates active targeting, which involves the use of targeting moieties that may be bound (either covalently or non-covalently) to the transfer vehicle to encourage localization of such transfer vehicle at certain target cells or target tissues. For example, targeting may be mediated by the inclusion of one or more endogenous targeting moieties in or on the transfer vehicle to encourage distribution to the target cells or tissues. Recognition of the targeting moiety by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes). As provided herein, the composition can comprise a moiety capable of enhancing affinity of the composition to the target cell. Targeting moieties may be linked to the outer bilayer of the lipid particle during formulation or post-formulation. These methods are well known in the art. In addition, some lipid particle formulations may employ fusogenic polymers such as PEAA, hemagluttinin, other lipopeptides (see U.S. patent application Ser. No. 08/835,281, and 60/083,294, which are incorporated herein by reference) and other features useful for in vivo and/or intracellular delivery. In other some embodiments, the compositions of the present invention demonstrate improved transfection efficacies, and/or demonstrate enhanced selectivity towards target cells or tissues of interest. Contemplated therefore are compositions which comprise one or more moieties (e.g., peptides, aptamers, oligonucleotides, a vitamin or other molecules) that are capable of enhancing the affinity of the compositions and their nucleic acid contents for the target cells or tissues. Suitable moieties may optionally be bound or linked to the surface of the transfer vehicle. In some embodiments, the targeting moiety may span the surface of a transfer vehicle or be encapsulated within the transfer vehicle. Suitable moieties and are selected based upon their physical, chemical or biological properties (e.g., selective affinity and/or recognition of target cell surface markers or features). Cell-specific target sites and their corresponding targeting ligand can vary widely. Suitable targeting moieties are selected such that the unique characteristics of a target cell are exploited, thus allowing the composition to discriminate between target and non-target cells. For example, compositions of the invention may include surface markers (e.g., apolipoprotein-B or apolipoprotein-E) that selectively enhance recognition of, or affinity to hepatocytes (e.g., by receptor-mediated recognition of and binding to such surface markers). As an example, the use of galactose as a targeting moiety would be expected to direct the compositions of the present invention to parenchymal hepatocytes, or alternatively the use of mannose containing sugar residues as a targeting ligand would be expected to direct the compositions of the present invention to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting moieties that have been conjugated to moieties present in the transfer vehicle (e.g., a lipid nanoparticle) therefore facilitate recognition and uptake of the compositions of the present invention in target cells and tissues. Examples of suitable targeting moieties include one or more peptides, proteins, aptamers, vitamins and oligonucleotides.

In particular embodiments, a transfer vehicle comprises a targeting moiety. In some embodiments, the targeting moiety mediates receptor-mediated endocytosis selectively into a specific population of cells. In some embodiments, the targeting moiety is capable of binding to a T cell antigen. In some embodiments, the targeting moiety is capable of binding to a NK, NKT, or macrophage antigen. In some embodiments, the targeting moiety is capable of binding to a protein selected from the group CD3, CD4, CD8, PD-1, 4-1BB, and CD2. In some embodiments, the targeting moiety is an single chain Fv (scFv) fragment, nanobody, peptide, peptide-based macrocycle, minibody, heavy chain variable region, light chain variable region or fragment thereof. In some embodiments, the targeting moiety is selected from T-cell receptor motif antibodies, T-cell α chain antibodies, T-cell R chain antibodies, T-cell γ chain antibodies, T-cell δ chain antibodies, CCR7 antibodies, CD3 antibodies, CD4 antibodies, CD5 antibodies, CD7 antibodies, CD8 antibodies, CD11b antibodies, CD11c antibodies, CD16 antibodies, CD19 antibodies, CD20 antibodies, CD21 antibodies, CD22 antibodies, CD25 antibodies, CD28 antibodies, CD34 antibodies, CD35 antibodies, CD40 antibodies, CD45RA antibodies, CD45RO antibodies, CD52 antibodies, CD56 antibodies, CD62L antibodies, CD68 antibodies, CD80 antibodies, CD95 antibodies, CD117 antibodies, CD127 antibodies, CD133 antibodies, CD137 (4-1BB) antibodies, CD163 antibodies, F4/80 antibodies, IL-4Rα antibodies, Sca-1 antibodies, CTLA-4 antibodies, GITR antibodies GARP antibodies, LAP antibodies, granzyme B antibodies, LFA-1 antibodies, transferrin receptor antibodies, and fragments thereof. In some embodiments, the targeting moiety is a small molecule binder of an ectoenzyme on lymphocytes. Small molecule binders of ectoenzymes include A2A inhibitors CD73 inhibitors, CD39 or adenosine receptors A2aR and A2bR. Potential small molecules include AB928.

In some embodiments, transfer vehicles are formulated and/or targeted as described in Shobaki N, Sato Y, Harashima H. Mixing lipids to manipulate the ionization status of lipid nanoparticles for specific tissue targeting. Int J Nanomedicine. 2018; 13:8395-8410. Published 2018 Dec. 10. In some embodiments, a transfer vehicle is made up of 3 lipid types. In some embodiments, a transfer vehicle is made up of 4 lipid types. In some embodiments, a transfer vehicle is made up of 5 lipid types. In some embodiments, a transfer vehicle is made up of 6 lipid types.

### B. Target Cells

Where it is desired to deliver a nucleic acid to an immune cell, the immune cell represents the target cell. In some embodiments, the compositions of the invention transfect the target cells on a discriminatory basis (i.e., do not transfect non-target cells). The compositions of the invention may also be prepared to preferentially target a variety of target cells, which include, but are not limited to, T cells, liver cells (e.g., hepatocytes), and muscle cells (e.g., myotubes), B cells, macrophages, and dendritic cells.

In some embodiments, the target cells are T cells, liver cells (e.g., hepatocytes), and muscle cells (e.g., myotubes). In some embodiments, the cells are primary cells (e.g., human primary cells). In some embodiments, the T cell is a CD8+ cytotoxic T cell, a CD4+ helper T cell (Th), a regulatory T (Treg) cell, a memory T cell, or an innate-like T cell. In some embodiments, the Th cell is a Th1 cell, a Th2 cell, a Th17 cell, a Th9 cell, a Tfh cell, or a Th22 cell. In some embodiments, the memory T cell is a central memory T cell (Tcm), an effector memory T cell (Tem), a tissue-resident memory T cell (Trm), or a virtual memory T cell. In some embodiments, the innate-like T cell is a natural killer T (NKT) cell, a mucosal-associated invariant T cell (MAIT), or a gamma delta T cell (γδ T cell). In some embodiments, the T cells are activated T cells (e.g., with an antigen-presenting cell (APC) or a CD3-stimulating agent and a CD28-stimulating agent). In some embodiments, activation is associated with production and release of cytokines, production of cytolytic enzymes, interaction with APCs, among others. In some embodiments, the T cells are not activated. In some embodiments, the target cells are deficient in a protein or enzyme of interest. For example, where it is desired to deliver a nucleic acid to a T cell, the T cell represents the target cell. In some embodiments, the compositions of the invention transfect the target cells on a discriminatory basis (i.e., do not transfect non-target cells).

In some embodiments, the target cells are deficient in a protein or enzyme of interest. For example, where it is desired to deliver a nucleic acid to a hepatocyte, the hepatocyte represents the target cell; where it is desired to deliver a nucleic acid to a T-cell, the T-cell represents the target cell. In some embodiments, the compositions of the invention transfect the target cells on a discriminatory basis (i.e., do not transfect non-target cells).

The compositions of the invention may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

The compositions of the invention may be prepared to preferentially distribute to target cells such as in the heart, lungs, kidneys, liver, and spleen. In some embodiments, the compositions of the invention distribute into the cells of the liver or spleen to facilitate the delivery and the subsequent expression of the circRNA comprised therein by the cells of the liver (e.g., hepatocytes) or the cells of spleen (e.g., immune cells). The compositions of the invention may be prepared to preferentially distribute to target cells such as in the blood, muscle, thymus, liver, and spleen. In some embodiments, the compositions of the invention distribute into the cells of the blood, liver, or spleen to facilitate the delivery and the subsequent expression of the circRNA comprised therein by the cells of or the cells of blood or spleen (e.g., immune cells), muscle (e.g., myotubes) or the liver (e.g., hepatocytes).

The targeted cells may function as a biological “reservoir” or “depot” capable of producing, and systemically excreting a functional protein or enzyme. Accordingly, in one embodiment of the invention the transfer vehicle may target hepatocytes or immune cells and/or preferentially distribute to the cells of the liver or spleen upon delivery. In an embodiment, following transfection of the target hepatocytes or immune cells, the circRNA loaded in the vehicle are translated and a functional protein product is produced, excreted and systemically distributed. In other embodiments, cells other than hepatocytes (e.g., lung, spleen, heart, ocular, or cells of the central nervous system) can serve as a depot location for protein production. In one embodiment of the invention the transfer vehicle may target T cells, myotubes, and hepatocytes. In an embodiment, following transfection of the target T cells, hepatocytes, and myotubes, the circRNA loaded in the vehicle are translated and a functional protein product is produced, excreted and systemically distributed.

In one embodiment, the compositions of the invention facilitate a subject's endogenous production of one or more functional proteins and/or enzymes. In an embodiment of the present invention, the transfer vehicles comprise circRNA which encode a deficient protein or enzyme. Upon distribution of such compositions to the target tissues and the subsequent transfection of such target cells, the exogenous circRNA loaded into the transfer vehicle (e.g., a lipid nanoparticle) may be translated in vivo to produce a functional protein or enzyme encoded by the exogenously administered circRNA (e.g., a protein or enzyme in which the subject is deficient). Accordingly, the compositions of the present invention exploit a subject's ability to translate exogenously- or recombinantly-prepared circRNA to produce an endogenously-translated protein or enzyme, and thereby produce (and where applicable excrete) a functional protein or enzyme. The expressed or translated proteins or enzymes may also be characterized by the in vivo inclusion of native post-translational modifications which may often be absent in recombinantly-prepared proteins or enzymes, thereby further reducing the immunogenicity of the translated protein or enzyme.

The administration of circRNA encoding a deficient protein or enzyme avoids the need to deliver the nucleic acids to specific organelles within a target cell. Rather, upon transfection of a target cell and delivery of the nucleic acids to the cytoplasm of the target cell, the circRNA contents of a transfer vehicle may be translated and a functional protein or enzyme expressed.

In some embodiments, a circular RNA comprises one or more miRNA binding sites. In some embodiments, a circular RNA comprises one or more miRNA binding sites recognized by miRNA present in one or more non-target cells or non-target cell types (e.g., Kupffer cells or hepatic cells) and not present in one or more target cells or target cell types (e.g., hepatocytes or T cells). In some embodiments, a circular RNA comprises one or more miRNA binding sites recognized by miRNA present in an increased concentration in one or more non-target cells or non-target cell types (e.g., Kupffer cells or hepatic cells) compared to one or more target cells or target cell types (e.g., hepatocytes or T cells). miRNAs are thought to function by pairing with complementary sequences within RNA molecules, resulting in gene silencing.

In some embodiments, the compositions of the invention transfect or distribute to target cells on a discriminatory basis (i.e. do not transfect non-target cells). The compositions of the invention may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells. In some embodiments, the compositions of the invention may be prepared to preferentially target a variety of target cells, which include T cells, hepatocytes, and muscle cells (e.g., myotubes).

### 9. Pharmaceutical Compositions

In certain embodiments, provided herein are compositions (e.g., pharmaceutical compositions) comprising a therapeutic agent provided herein. In some embodiments, the therapeutic agent is a circular RNA polynucleotide provided herein. In some embodiments the therapeutic agent is a vector provided herein. In some embodiments, the therapeutic agent is a cell comprising a circular RNA or vector provided herein (e.g., a human cell, such as a human T-cell). In certain embodiments, the composition further comprises a pharmaceutically acceptable carrier. In some embodiments, the compositions provided herein comprise a therapeutic agent provided herein in combination with other pharmaceutically active agents or drugs, such as anti-inflammatory drugs or antibodies capable of targeting B cell antigens, e.g., anti-CD20 antibodies, e.g., rituximab.

With respect to pharmaceutical compositions, the pharmaceutically acceptable carrier can be any of those conventionally used and is limited only by physico-chemical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions provided herein.

In certain embodiments, the pharmaceutical composition comprises a preservative. In certain embodiments, suitable preservatives may include, for example, methylparaben, propylparaben, sodium benzoate, and benzalkonium chloride. Optionally, a mixture of two or more preservatives may be used. The preservative or mixtures thereof are typically present in an amount of about 0.0001% to about 2% by weight of the total composition.

In some embodiments, the pharmaceutical composition comprises a buffering agent. In some embodiments, suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

In some embodiments, the concentration of therapeutic agent in the pharmaceutical composition can vary, e.g., less than about 1%, or at least about 1%, 2%, 3%, 4%, 5%, 6%, 7%, 8%9% 10%%, 15%, 20%, 25%, 30%, 35%, 40%, 45%, or about 50% or more by weight, and can be selected primarily by fluid volumes, and viscosities, in accordance with the particular mode of administration selected.

The following formulations for oral, aerosol, parenteral (e.g., subcutaneous, intravenous, intraarterial, intramuscular, intradermal, intraperitoneal, and intrathecal), and topical administration are merely exemplary and are in no way limiting. More than one route can be used to administer the therapeutic agents provided herein, and in certain instances, a particular route can provide a more immediate and more effective response than another route.

Formulations suitable for oral administration can comprise or consist of (a) liquid solutions, such as an effective amount of the therapeutic agent dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the therapeutic agent with a flavorant, usually sucrose, acacia or tragacanth. Pastilles can comprise the therapeutic agent with an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In some embodiments, the therapeutic agents provided herein can be administered in a physiologically acceptable diluent in a pharmaceutical carrier, such as a sterile liquid or mixture of liquids including water, saline, aqueous dextrose and related sugar solutions, an alcohol such as ethanol or hexadecyl alcohol, a glycol such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agents and other pharmaceutical adjuvants.

Oils, which can be used in parenteral formulations in some embodiments, include petroleum, animal oils, vegetable oils, or synthetic oils. Specific examples of oils include peanut, soybean, sesame, cottonseed, corn, olive, petrolatum, and mineral oil. Suitable fatty acids for use in parenteral formulations include oleic acid, stearic acid, and isostearic acid. Ethyl oleate and isopropyl myristate are examples of suitable fatty acid esters.

Suitable soaps for use in certain embodiments of parenteral formulations include fatty alkali metal, ammonium, and triethanolamine salts, and suitable detergents include (a) cationic detergents such as, for example, dimethyl dialkyl ammonium halides. and alkyl pyridinium halides, (b) anionic detergents such as, for example, alkyl, aryl, and olefin sulfonates, alky, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

In some embodiments, the parenteral formulations will contain, for example, from about 0.5% to about 25% by weight of the therapeutic agent in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol, sorbitan, fatty acid esters such as sorbitan monooleate, and high molecular weight adducts of ethylene oxide with a hydrophobic base formed by the condensation of propylene oxide with propylene glycol. The parenteral formulations can be presented in unit-dose or multi-dose sealed containers, such as ampoules or vials, and can be stored in a freeze-dried (lyophilized) condition requiring only the addition of a sterile liquid excipient, for example, water for injections, immediately prior to use. Extemporaneous injection solutions and suspensions can be prepared from sterile powders, granules, and tablets of the kind previously described.

In certain embodiments, injectable formulations are provided herein. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed, pages 622-630 (1986)).

In some embodiments, topical formulations are provided herein. Topical formulations, including those that are useful for transdermal drug release, are suitable in the context of certain embodiments provided herein for application to skin. In some embodiments, the therapeutic agent alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

In certain embodiments, the therapeutic agents provided herein can be formulated as inclusion complexes, such as cyclodextrin inclusion complexes, or liposomes. Liposomes can serve to target the therapeutic agents to a particular tissue. Liposomes also can be used to increase the half-life of the therapeutic agents. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

In some embodiments, the therapeutic agents provided herein are formulated in time-released, delayed release, or sustained release delivery systems such that the delivery of the composition occurs prior to, and with sufficient time to, cause sensitization of the site to be treated. Such systems can avoid repeated administrations of the therapeutic agent, thereby increasing convenience to the subject and the physician, and may be particularly suitable for certain composition embodiments provided herein. In one embodiment, the compositions of the invention are formulated such that they are suitable for extended-release of the circRNA contained therein. Such extended-release compositions may be conveniently administered to a subject at extended dosing intervals. For example, in one embodiment, the compositions of the present invention are administered to a subject twice a day, daily or every other day. In an embodiment, the compositions of the present invention are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by an inventive polynucleotide is produced by a target cell for sustained amounts of time. For example, the protein may be produced for more than one hour, more than four, more than six, more than 12, more than 24, more than 48 hours, or more than 72 hours after administration. In some embodiments the polypeptide is expressed at a peak level about six hours after administration. In some embodiments the expression of the polypeptide is sustained at least at a therapeutic level. In some embodiments, the polypeptide is expressed at least at a therapeutic level for more than one, more than four, more than six, more than 12, more than 24, more than 48, or more than 72 hours after administration. In some embodiments, the polypeptide is detectable at a therapeutic level in patient tissue (e.g., liver or lung). In some embodiments, the level of detectable polypeptide is from continuous expression from the circRNA composition over periods of time of more than one, more than four, more than six, more than 12, more than 24, more than 48, or more than 72 hours after administration.

In certain embodiments, a protein encoded by an inventive polynucleotide is produced at levels above normal physiological levels. The level of protein may be increased as compared to a control. In some embodiments, the control is the baseline physiological level of the polypeptide in a normal individual or in a population of normal individuals. In other embodiments, the control is the baseline physiological level of the polypeptide in an individual having a deficiency in the relevant protein or polypeptide or in a population of individuals having a deficiency in the relevant protein or polypeptide. In some embodiments, the control can be the normal level of the relevant protein or polypeptide in the individual to whom the composition is administered. In other embodiments, the control is the expression level of the polypeptide upon other therapeutic intervention, e.g., upon direct injection of the corresponding polypeptide, at one or more comparable time points.

In certain embodiments, the levels of a protein encoded by an inventive polynucleotide are detectable at 3 days, 4 days, 5 days, or 1 week or more after administration. Increased levels of protein may be observed in a tissue (e.g., liver or lung).

In some embodiments, the method yields a sustained circulation half-life of a protein encoded by an inventive polynucleotide. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more. In some embodiments, the functional half-life of the protein is at least 6 hours (e.g., at least 6, 7, 8, 9, 10, 12, 14, 16, 18, 20, 22, 24, 36, 48 hours, or more).

Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems: wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

In some embodiments, the therapeutic agent can be conjugated either directly or indirectly through a linking moiety to a targeting moiety. Methods for conjugating therapeutic agents to targeting moieties is known in the art. See, for instance, Wadwa et al., J, Drug Targeting 3:111 (1995) and U.S. Pat. No. 5,087,616.

In some embodiments, the therapeutic agents provided herein are formulated into a depot form, such that the manner in which the therapeutic agent is released into the body to which it is administered is controlled with respect to time and location within the body (see, for example, U.S. Pat. No. 4,450,150). Depot forms of therapeutic agents can be, for example, an implantable composition comprising the therapeutic agents and a porous or non-porous material, such as a polymer, wherein the therapeutic agents are encapsulated by or diffused throughout the material and/or degradation of the non-porous material. The depot is then implanted into the desired location within the body and the therapeutic agents are released from the implant at a predetermined rate.

### 10. Therapeutic Methods

### A. Areas for Treatment & Relevant Diseases/Disorders

Also provided herein is a method of treating a subject in need thereof comprising administering a therapeutically effective amount of the circular RNA provided herein. In some embodiments, provided herein is a method of treating a subject in need thereof comprising administering a therapeutically effective amount of a composition comprising the circular RNA provided herein. In some embodiments, in addition to the circular RNA, a delivery vehicle, and optionally, a targeting moiety operably connected to the delivery vehicle is administered.

In some embodiments, provided herein are methods of treating and/or preventing a disease in a subject (e.g., mammalian subject, such as a human subject).

In some embodiments, provided herein are methods of treating and/or preventing a disease in a subject (e.g., mammalian subject, such as a human subject) by introducing or administering an effective amount of a pharmaceutical composition comprising a circular RNA encoding a therapeutic protein, e.g., dystrophin, and at least one delivery vehicle as described herein.

In some embodiments, provided herein is a method of vaccinating a subject by introducing or administering an effective amount of a pharmaceutical composition described herein comprising the circular RNA described herein and at least one delivery vehicle as described herein. In some embodiments, the method of vaccinating comprises administering an effective amount of an antigen comprising a viral polypeptide from an adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumo virus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; Japanese encephalitis virus; Vesicular exanthernavirus; SARS-CoV-2; Eastern equine encephalitis, or a combination of any two or more of the foregoing.

In some embodiments, the circular RNA encodes a CAR, the CARs have biological activity, e.g., ability to recognize an antigen, e.g., CD19, HER2, or BCMA, such that the CAR, when expressed by a cell, is able to mediate an immune response against the cell expressing the antigen, e.g., CD19, HER2, or BCMA, for which the CAR is specific. In this regard, an embodiment provided herein provides a method of treating or preventing cancer in a subject, comprising administering to the subject the circular RNA therapeutic agents, and/or the pharmaceutical compositions provided herein in an amount effective to treat or prevent cancer in the subject.

In some embodiments, the subject has a cancer selected from the group consisting of: acute myeloid leukemia (AML); alveolar rhabdomyosarcoma; B cell malignancies; bladder cancer (e.g., bladder carcinoma); bone cancer; brain cancer (e.g., medulloblastoma and glioblastoma multiforme); breast cancer; cancer of the anus, anal canal, or anorectum; cancer of the eye; cancer of the intrahepatic bile duct; cancer of the joints; cancer of the neck; gallbladder cancer; cancer of the pleura; cancer of the nose, nasal cavity, or middle ear; cancer of the oral cavity; cancer of the vulva; chronic lymphocytic leukemia; chronic myeloid cancer; colon cancer; esophageal cancer, cervical cancer; fibrosarcoma; gastrointestinal carcinoid tumor; head and neck cancer (e.g., head and neck squamous cell carcinoma); Hodgkin lymphoma; hypopharynx cancer; kidney cancer; larynx cancer; leukemia; liquid tumors; lipoma; liver cancer; lung cancer (e.g., non-small cell lung carcinoma, lung adenocarcinoma, and small cell lung carcinoma); lymphoma; mesothelioma; mastocytoma; melanoma; multiple myeloma; nasopharynx cancer; non-Hodgkin lymphoma; B-chronic lymphocytic leukemia; hairy cell leukemia; Burkitt's lymphoma; ovarian cancer; pancreatic cancer; cancer of the peritoneum; cancer of the omentum; mesentery cancer; pharynx cancer; prostate cancer; rectal cancer; renal cancer; skin cancer; small intestine cancer; soft tissue cancer; solid tumors; synovial sarcoma; gastric cancer; teratoma; testicular cancer; thyroid cancer; and ureter cancer.

In some embodiments, the subject has an autoimmune disease or disorder.

In some embodiments, the subject has an autoimmune disease or disorder selected from scleroderma, Grave's disease, Crohn's disease, Sjogren's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, thyroiditis, and the generalized autoimmune diseases typified by human Lupus.

In some embodiments, the subject has an autoimmune disease or disorder that is B-cell mediated. In some embodiments, the subject has an autoimmune disease or disorder that is autoantibody mediated.

In some embodiments, the subject has lupus, e.g., systemic lupus erythematosus (SLE), cutaneous lupus erythematosus (CLE), lupus nephritis (LN), neonatal lupus, drug-induced lupus.

In the subject has antisynthetase syndrome, multifocal motor neuropathy, myasthenia gravis, neuromyelitis optica, pemphigus vulgaris, and/or systemic sclerosis.

In some embodiments, provided herein is a method of treating and/or preventing cancer in a subject, comprising introducing the circular RNA and/or pharmaceutical composition thereof disclosed herein. In some embodiments, provided herein is a method of treating cancer in a subject, comprising introducing the circular RNA encoding CD19 CAR and/or pharmaceutical composition thereof disclosed herein. In some embodiments, provided herein is a method of treating cancer in a subject, comprising introducing the circular RNA encoding HER2 CAR and/or pharmaceutical composition thereof disclosed herein. In some embodiments, provided herein is a method of treating cancer in a subject, comprising introducing the circular RNA encoding BCMA CAR and/or pharmaceutical composition thereof disclosed herein.

In some embodiments, provided herein is a method of treating and/or preventing B-cell lymphoma, e.g., large B-cell lymphoma, in a subject, comprising introducing the circular RNA and/or pharmaceutical composition thereof disclosed herein. In some embodiments, provided herein is a method of treating B-cell lymphoma, e.g., large B-cell lymphoma, in a subject, comprising introducing the circular RNA encoding CD19 CAR and/or pharmaceutical composition thereof disclosed herein. In some embodiments, provided herein is a method of treating B-cell lymphoma, e.g., large B-cell lymphoma, in a subject, comprising introducing the circular RNA encoding CD19 CAR and comprising an IRES and/or pharmaceutical composition thereof disclosed herein.

In some embodiments, provided herein is a method of treating and/or preventing mantle cell lymphoma in a subject, comprising introducing the circular RNA and/or pharmaceutical composition thereof disclosed herein. In some embodiments, provided herein is a method of treating mantle cell lymphoma in a subject, comprising introducing the circular RNA encoding CD19 CAR and/or pharmaceutical composition thereof disclosed herein. In some embodiments, provided herein is a method of treating mantle cell lymphoma in a subject, comprising introducing the circular RNA encoding CD19 CAR and comprising an IRES and/or pharmaceutical composition thereof disclosed herein.

In some embodiments, provided herein is a method of treating and/or preventing multiple myeloma in a subject, comprising introducing the circular RNA and/or pharmaceutical composition thereof disclosed herein. In some embodiments, provided herein is a method of treating multiple myeloma in a subject, comprising introducing the circular RNA encoding BCMA CAR and/or pharmaceutical composition thereof disclosed herein. In some embodiments, provided herein is a method of treating multiple myeloma in a subject, comprising introducing the circular RNA encoding BCMA CAR and comprising an IRES and/or pharmaceutical composition thereof disclosed herein.

In some embodiments, provided herein is a method of treating and/or preventing a solid tumor in a subject, comprising introducing the circular RNA and/or pharmaceutical composition thereof disclosed herein. In some embodiments, provided herein is a method of treating a solid tumor in a subject, comprising introducing the circular RNA encoding HER2 CAR and/or pharmaceutical composition thereof disclosed herein. In some embodiments, provided herein is a method of treating a solid tumor in a subject, comprising introducing the circular RNA encoding HER2 CAR and comprising an IRES and/or pharmaceutical composition thereof disclosed herein.

In 2022, Mackensen et al. reported a compassionate-use anti-CD19 CAR T cell therapy for refractory systemic lupus erythematosus in which autologous T cells from SLE patients “were transduced with a lentiviral anti-CD19 CAR vector, expanded and reinfused . . . into the patients after lymphodepletion . . . CAR T cells expanded in vivo, led to deep depletion of B cells, improvement of clinical symptoms and normalization of laboratory parameters including seroconversion of anti-double-stranded DNA antibodies. Remission of SLE according to DORIS criteria was achieved in all five patients after 3 months and the median (range) Systemic Lupus Erythematosus Disease Activity Index score after 3 months was 0 (2).” Mackensen et al., Anti-CD19 CAR T cell therapy for refractory systemic lupus erythematosus, Nature Medicine (2022); see also Nunez et al., Cytokine and reactivity profiles in SLE patients following anti-CD19 CART therapy, Molecular Therapy (2023); the contents of both of which are hereby incorporated by reference in their entireties.

In some embodiments, provided herein is a method of treating and/or preventing an autoimmune disease, e.g., a B cell mediated autoimmune disease, e.g., lupus, in a subject, comprising introducing the circular RNA encoding CD19 CAR and/or pharmaceutical composition thereof disclosed herein. In some embodiments, provided herein is a method of treating lupus in a subject, comprising introducing the circular RNA encoding CD19 CAR and/or pharmaceutical composition thereof disclosed herein. In some embodiments, provided herein is a method of treating SLE in a subject, comprising introducing the circular RNA encoding CD19 CAR and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA encoding CD19 CAR and comprising an IRES and/or pharmaceutical composition thereof disclosed herein. In some embodiments, provided herein is a method of treating lupus in a subject, comprising introducing the circular RNA encoding CD19 CAR and comprising an IRES and/or pharmaceutical composition thereof disclosed herein. In some embodiments, provided herein is a method of treating SLE in a subject, comprising introducing the circular RNA encoding CD19 CAR and comprising an IRES and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA and/or pharmaceutical composition thereof disclosed herein. In some embodiments, provided herein is a method of treating lupus in a subject, comprising introducing the circular RNA and/or pharmaceutical composition thereof disclosed herein. In some embodiments, provided herein is a method of treating SLE in a subject, comprising introducing the circular RNA and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA encoding CD19 CAR and/or pharmaceutical composition thereof disclosed herein. In some embodiments, provided herein is a method of treating lupus in a subject, comprising introducing the circular RNA encoding CD19 CAR and/or pharmaceutical composition thereof disclosed herein. In some embodiments, provided herein is a method of treating SLE in a subject, comprising introducing the circular RNA encoding CD19 CAR and/or pharmaceutical composition thereof disclosed herein.

In some embodiments, provided herein is a method of treating and/or preventing an autoimmune disease, e.g., a B cell mediated autoimmune disease, e.g., lupus, in a subject, comprising introducing the circular RNA encoding CD19 CAR and comprising an IRES and/or pharmaceutical composition thereof disclosed herein. In some embodiments, provided herein is a method of treating lupus in a subject, comprising introducing the circular RNA encoding CD19 CAR and comprising an IRES and/or pharmaceutical composition thereof disclosed herein. In some embodiments, provided herein is a method of treating SLE in a subject, comprising introducing the circular RNA encoding CD19 CAR and comprising an IRES and/or pharmaceutical composition thereof disclosed herein.

The transfer vehicles may preferentially distribute to specific target cells such as immune cells (e.g., T cells, NK cells, macrophages, etc.), or in the heart, lungs, kidneys, liver, and spleen. In some embodiments, the compositions distribute into the cells of the liver or spleen to facilitate the delivery and the subsequent expression of the circular RNA comprised therein by the cells of the liver (e.g., hepatocytes) or the cells of spleen (e.g., immune cells). The targeted cells may function as a biological “reservoir” or “depot” capable of producing, and systemically excreting a functional protein or enzyme. Accordingly, in one embodiment of the disclosure, the transfer vehicle may target hepatocytes or immune cells and/or preferentially distribute to the cells of the liver or spleen upon delivery. In an embodiment, following transfection of the target hepatocytes or immune cells, the circular RNA loaded in the vehicle is translated and a functional protein product is produced, excreted and systemically distributed. In other embodiments, cells other than hepatocytes (e.g., lung, spleen, heart, ocular, or cells of the central nervous system) can serve as a depot location for protein production. The compositions may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

In some embodiments, provided herein is a method of treating and/or preventing a disease or disorder in a subject, comprising introducing the circular RNA and/or pharmaceutical composition thereof to an immune cell in the subject.

In some embodiments, provided herein is a method of treating and/or preventing a disease or disorder in a subject, comprising introducing the circular RNA and/or pharmaceutical composition thereof to a non-immune cell in the subject, e.g., a muscle or liver cell.

In some embodiments, provided herein is a method of treating and/or preventing a disease or disorder in a subject, comprising introducing the circular RNA and/or pharmaceutical composition thereof to a T cell in the subject. In some embodiments, provided herein is a method of treating a disease or disorder in a subject, comprising introducing the circular RNA and/or pharmaceutical composition thereof to a helper T cell in the subject. In some embodiments, provided herein is a method of treating a disease or disorder in a subject, comprising introducing the circular RNA and/or pharmaceutical composition thereof to a cytotoxic T cell in the subject.

In some embodiments, provided herein is a method of treating and/or preventing a disease or disorder in a subject, comprising introducing the circular RNA and/or pharmaceutical composition thereof to a NK cell in the subject.

In some embodiments, provided herein is a method of treating and/or preventing a disease or disorder in a subject, comprising introducing the circular RNA and/or pharmaceutical composition thereof to a macrophage in the subject.

In some embodiments, provided herein is a method of treating and/or preventing a disease or disorder in a subject, comprising introducing the circular RNA and/or pharmaceutical composition thereof to a myeloid cell in the subject.

In some embodiments, provided herein is a method of treating and/or preventing a disease or disorder in a subject, comprising introducing the circular RNA and/or pharmaceutical composition thereof to a monocyte in the subject.

In some embodiments, provided herein is a method of treating and/or preventing a disease or disorder in a subject, comprising introducing the circular RNA and/or pharmaceutical composition thereof to a CD3+ cell in the subject.

In some embodiments, provided herein is a method of treating and/or preventing a disease or disorder in a subject, comprising introducing the circular RNA and/or pharmaceutical composition thereof to a CD4+ cell in the subject.

In some embodiments, provided herein is a method of treating and/or preventing a disease or disorder in a subject, comprising introducing the circular RNA and/or pharmaceutical composition thereof to a CD8+ cell in the subject.

In some embodiments, provided herein is a method of treating and/or preventing a disease or disorder in a subject, comprising introducing the circular RNA and/or pharmaceutical composition thereof to a CD14+ cell in the subject.

In some embodiments, provided herein is a method of treating and/or preventing a disease or disorder in a subject, comprising introducing the circular RNA and/or pharmaceutical composition thereof to a CD16+ cell in the subject.

In some embodiments, provided herein is a method of treating and/or preventing a disease or disorder in a subject, comprising introducing the circular RNA and/or pharmaceutical composition thereof to a CD33+ cell in the subject.

In some embodiments, provided herein is a method of treating and/or preventing a disease or disorder in a subject, comprising introducing the circular RNA and/or pharmaceutical composition thereof to a CD33+CD14+ cell in the subject.

In some embodiments, provided herein is a method of treating and/or preventing a disease or disorder in a subject, comprising introducing the circular RNA and/or pharmaceutical composition thereof to a CD33+CD64+ cell in the subject.

In some embodiments, provided herein is a method of treating and/or preventing a disease or disorder in a subject, comprising introducing the circular RNA and/or pharmaceutical composition thereof to a CD56+ cell in the subject.

In some embodiments, provided herein is a method of treating and/or preventing a disease or disorder in a subject, comprising introducing the circular RNA and/or pharmaceutical composition thereof to a CD11B+ cell in the subject.

In certain embodiments the therapeutic agents provided herein (i.e., circular RNA constructs and compositions thereof) and the transfer vehicles (e.g., lipid nanoparticles) exhibit an enhanced (e.g., increased) ability to transfect one or more target cells. Accordingly, also provided herein are methods of transfecting one or more target cells. Such methods generally comprise the step of contacting the one or more target cells with the compounds and/or pharmaceutical compositions disclosed herein such that the one or more target cells are transfected with the circular RNA encapsulated therein. As used herein, the terms “transfect” or “transfection” refer to the intracellular introduction of one or more encapsulated materials (e.g., nucleic acids and/or polynucleotides) into a cell, or preferably into a target cell. The term “transfection efficiency” refers to the relative amount of such encapsulated material (e.g., polynucleotides) up-taken by, introduced into and/or expressed by the target cell which is subject to transfection. In some embodiments, transfection efficiency may be estimated by the amount of a reporter polynucleotide product produced by the target cells following transfection. In some embodiments, a transfer vehicle has high transfection efficiency. In some embodiments, a transfer vehicle has at least about 10%, 20%, 30%, 40%, 50%, 60%, 70%, 80%, or 90% transfection efficiency.

In certain embodiments, the therapeutic agents provided herein (i.e., circular RNA constructs and compositions thereof) are co-administered with one or more additional therapeutic agents e.g., in the same pharmaceutical composition or in separate pharmaceutical compositions. In some embodiments, the therapeutic agent provided herein can be administered first and the one or more additional therapeutic agents can be administered second, or vice versa. Alternatively, the therapeutic agent provided herein and the one or more additional therapeutic agents can be administered simultaneously. In some embodiments, the therapeutic agents are co-administered sufficiently close in time such that the therapeutic agent provided herein can enhance the effect of the one or more additional therapeutic agents, or vice versa.

In some embodiments, the subject is a mammal. In some embodiments, the mammal referred to herein can be any mammal, including, but not limited to, mammals of the order Rodentia, such as mice and hamsters, or mammals of the order Logomorpha, such as rabbits. The mammals may be from the order Carnivora, including Felines (cats) and Canines (dogs). The mammals may be from the order Artiodactyla, including Bovines (cows) and Swines (pigs), or of the order Perssodactyla, including Equines (horses). The mammals may be of the order Primates, Ceboids, or Simoids (monkeys) or of the order Anthropoids (humans and apes). Preferably, the mammal is a human.

### EXAMPLES

Wesselhoeft et al., (2019) RNA Circularization Diminishes Immunogenicity and Can Extend Translation Duration In vivo. Molecular Cell. 74(3), 508-520 and Wesselhoeft et al., (2018) Engineering circular RNA for Potent and Stable Translation in Eukaryotic Cells. Nature Communications. 9, 2629 are incorporated by reference in their entirety.

The invention is further described in detail by reference to the following examples but are not intended to be limited to the following examples. These examples encompass any and all variations of the illustrations with the intention of providing those of ordinary skill in the art with complete disclosure and description of how to make and use the subject invention and are not intended to limit the scope of what is regarded as the invention.

### Example 1

### Example 1a: External Duplex Regions Allow for Circularization of Long Precursor RNA Using the Permuted Intron Exon (PIE) Circularization Strategy

A 1.1 kb sequence containing a full-length encephalomyocarditis virus (EMCV) IRES, a Gaussia luciferase (GLuc) expression sequence, and two short exon segments of the permuted intron-exon (PIE) construct were inserted between the 3′ and 5′ introns of the permuted group I catalytic intron in the thymidylate synthase (Td) gene of the T4 phage. Precursor RNA was synthesized by run-off transcription. Circularization was attempted by heating the precursor RNA in the presence of magnesium ions and GTP, but splicing products were not obtained.

Perfectly complementary 9 nucleotide and 19 nucleotide long duplex regions were designed and added at the 5′ and 3′ ends of the precursor RNA. Addition of these homology arms increased splicing efficiency from 0 to 16% for 9 nucleotide duplex regions and to 48% for 19 nucleotide duplex regions as assessed by disappearance of the precursor RNA band.

The splicing product was treated with RNase R. Sequencing across the putative splice junction of RNase R-treated splicing reactions revealed ligated exons, and digestion of the RNase R-treated splicing reaction with oligonucleotide-targeted RNase H produced a single band in contrast to two bands yielded by RNase H-digested linear precursor. This shows that circular RNA is a major product of the splicing reactions of precursor RNA containing the 9 or 19 nucleotide long external duplex regions

### Example 1B: Spacers that Conserve Secondary Structures of IRES and PIE Splice Sites Increase Circularization Efficiency

A series of spacers was designed and inserted between the 3′ PIE splice site and the TRES. These spacers were designed to either conserve or disrupt secondary structures within intron sequences in the IRES, 3′ PIE splice site, and/or 5′ splice site. The addition of spacer sequences designed to conserve secondary structures resulted in 87% splicing efficiency, while the addition of a disruptive spacer sequences resulted in no detectable splicing.

### Example 2

### Example 2a: Internal Duplex Regions in Addition to External Duplex Regions Create a Splicing Bubble and Allows for Translation of Several Expression Sequences

Spacers were designed to be unstructured, non-homologous to the intron and IRES sequences, and to contain spacer-spacer duplex regions. These were inserted between the 5′ exon and IRES and between the 3′ exon and expression sequence in constructs containing external duplex regions, EMCV IRES, and expression sequences for Gaussia luciferase (total length: 1289 nt), Firefly luciferase (2384 nt), eGFP (1451 nt), human erythropoietin (1313 nt), and Cas9 endonuclease (4934 nt). Circularization of all 5 constructs was achieved. Circularization of constructs utilizing T4 phage and Anabaena introns were roughly equal. Circularization efficiency was higher for shorter sequences. To measure translation, each construct was transfected into HEK293 cells. Gaussia and Firefly luciferase transfected cells produced a robust response as measured by luminescence, human erythropoietin was detectable in the media of cells transfected with erythropoietin circRNA, and EGFP fluorescence was observed from cells transfected with EGFP circRNA. Co-transfection of Cas9 circRNA with sgRNA directed against GFP into cells constitutively expressing GFP resulted in ablated fluorescence in up to 97% of cells in comparison to an sgRNA-only control.

### Example 2B: Use of CVB3 IRES Increases Protein Production

Constructs with internal and external duplex regions and differing IRES containing either Gaussia luciferase or Firefly luciferase expression sequences were made. Protein production was measured by luminescence in the supernatant of HEK293 cells 24 hours after transfection. The Coxsackievirus B3 (CVB3) IRES construct produced the most protein in both cases.

### Example 2C: Use of polyA or polyAC Spacers Increases Protein Production

Thirty nucleotide long polyA or polyAC spacers were added between the IRES and splice junction in a construct with each IRES that produced protein in example 2B. Gaussia luciferase activity was measured by luminescence in the supernatant of HEK293 cells 24 hours after transfection. Both spacers improved expression in every construct over control constructs without spacers.

### Example 3

Hek293 or HeLa Cells Transfected with Circular RNA Produce More Protein than Those Transfected with Comparable Unmodified or Modified Linear RNA.

HPLC-purified Gaussia luciferase-coding circRNA (CVB3-GLuc-pAC) was compared with a canonical unmodified 5′ methylguanosine-capped and 3′ polyA-tailed linear GLuc mRNA, and a commercially available nucleoside-modified (pseudouridine, 5-methylcytosine) linear GLuc mRNA (from Trilink). Luminescence was measured 24 h post-transfection, revealing that circRNA produced 811.2% more protein than the unmodified linear mRNA in HEK293 cells and 54.5% more protein than the modified mRNA. Similar results were obtained in HeLa cells and a comparison of optimized circRNA coding for human erythropoietin with linear mRNA modified with 5-methoxyuridine.

Luminescence data was collected over 6 days. In HEK293 cells, circRNA transfection resulted in a protein production half-life of 80 hours, in comparison with the 43 hours of unmodified linear mRNA and 45 hours of modified linear mRNA. In HeLa cells, circRNA transfection resulted in a protein production half-life of 116 hours, in comparison with the 44 hours of unmodified linear mRNA and 49 hours of modified linear mRNA. CircRNA produced substantially more protein than both the unmodified and modified linear mRNAs over its lifetime in both cell types.

### Example 4

### Example 4a: Purification of circRNA by RNase Digestion, HPLC Purification, and Phosphatase Treatment Decreases Immunogenicity. Completely Purified Circular RNA is Significantly Less Immunogenic than Unpurified or Partially Purified Circular RNA. Protein Expression Stability and Cell Viability are Dependent on Cell Type and Circular RNA Purity

Human embryonic kidney 293 (HEK293) and human lung carcinoma A549 cells were transfected with:
a. products of an unpurified GLuc circular RNA splicing reaction,
b. products of RNase R digestion of the splicing reaction,
c. products of RNase R digestion and HPLC purification of the splicing reaction, or
d. products of RNase digestion, HPLC purification, and phosphatase treatment of the splicing reaction.

RNase R digestion of splicing reactions was insufficient to prevent cytokine release in A549 cells in comparison to untransfected controls.

The addition of HPLC purification was also insufficient to prevent cytokine release, although there was a significant reduction in interleukin-6 (IL-6) and a significant increase in interferon-α1 (IFNα1) compared to the unpurified splicing reaction.

The addition of a phosphatase treatment after HPLC purification and before RNase R digestion dramatically reduced the expression of all upregulated cytokines assessed in A549 cells. Secreted monocyte chemoattractant protein 1 (MCP1), IL-6, IFNα1, tumor necrosis factor α (TNFα), and IFNγ inducible protein-10 (IP-10) fell to undetectable or untransfected baseline levels.

There was no substantial cytokine release in HEK293 cells. A549 cells had increased GLuc expression stability and cell viability when transfected with higher purity circular RNA. Completely purified circular RNA had a stability phenotype similar to that of transfected 293 cells.

### Example 4B: Circular RNA Does not Cause Significant Immunogenicity and is not a RIG-I Ligand

A549 cells were transfected with:
a. unpurified circular RNA,
b. high molecular weight (linear and circular concatenations) RNA,
c. circular (nicked) RNA,
d. an early fraction of purified circular RNA (more overlap with nicked RNA peak),
e. a late fraction of purified circular RNA (less overlap with nicked RNA peak),
f. introns excised during circularization, or
g. vehicle (i.e. untransfected control).

Precursor RNA was separately synthesized and purified in the form of the splice site deletion mutant (DS) due to difficulties in obtaining suitably pure linear precursor RNA from the splicing reaction. Cytokine release and cell viability was measured in each case.

Robust IL-6, RANTES, and IP-10 release was observed in response to most of the species present within the splicing reaction, as well as precursor RNA. Early circRNA fractions elicited cytokine responses comparable to other non-circRNA fractions, indicating that even relatively small quantities of linear RNA contaminants are able to induce a substantial cellular immune response in A549 cells. Late circRNA fractions elicited no cytokine response in excess of that from untransfected controls. A549 cell viability 36 hours post-transfection was significantly greater for late circRNA fractions compared with all of the other fractions.

RIG-I and IFN-β 1 transcript induction upon transfection of A549 cells with late circRNA HPLC fractions, precursor RNA or unpurified splicing reactions were analyzed. Induction of both RIG-I and IFN-β1 transcripts were weaker for late circRNA fractions than precursor RNA and unpurified splicing reactions. RNase R treatment of splicing reactions alone was not sufficient to ablate this effect. Addition of very small quantities of the RIG-I ligand 3p-hpRNA to circular RNA induced substantial RIG-I transcription. In HeLa cells, transfection of RNase R-digested splicing reactions induced RIG-I and IFN-β1, but purified circRNA did not. Overall, HeLa cells were less sensitive to contaminating RNA species than A549 cells.

A time course experiment monitoring RIG-I, IFN-β1, IL-6, and RANTES transcript induction within the first 8 hours after transfection of A549 cells with splicing reactions or fully purified circRNA did not reveal a transient response to circRNA. Purified circRNA similarly failed to induce pro-inflammatory transcripts in RAW264.7 murine macrophages.

A549 cells were transfected with purified circRNA containing an EMCV IRES and EGFP expression sequence. This failed to produce substantial induction of pro-inflammatory transcripts. These data demonstrate that non-circular components of the splicing reaction are responsible for the immunogenicity observed in previous studies and that circRNA is not a natural ligand for RIG-I.

### Example 5

### Circular RNA Avoids Detection by TLRs.

TLR3, 7, and 8 reporter cell lines were transfected with multiple linear or circular RNA constructs and secreted embryonic alkaline phosphatase (SEAP) was measured.

Linearized RNA was constructed by deleting the intron and homology arm sequences. The linear RNA constructs were then treated with phosphatase (in the case of capped RNAs, after capping) and purified by HPLC.

None of the attempted transfections produced a response in TLR7 reporter cells. TLR3 and TLR8 reporter cells were activated by capped linearized RNA, polyadenylated linearized RNA, the nicked circRNA HPLC fraction, and the early circRNA fraction. The late circRNA fraction and mlW-mRNA did not provoke TLR-mediated response in any cell line.

In a second experiment, circRNA was linearized using two methods: treatment of circRNA with heat in the presence of magnesium ions and DNA oligonucleotide-guided RNase H digestion. Both methods yielded a majority of full-length linear RNA with small amounts of intact circRNA. TLR3, 7, and 8 reporter cells were transfected with circular RNA, circular RNA degraded by heat, or circular RNA degraded by RNase H, and SEAP secretion was measured 36 hours after transfection. TLR8 reporter cells secreted SEAP in response to both forms of degraded circular RNA, but did not produce a greater response to circular RNA transfection than mock transfection. No activation was observed in TLR3 and TLR7 reporter cells for degraded or intact conditions, despite the activation of TLR3 by in vitro transcribed linearized RNA.

### Example 6

Unmodified Circular RNA Produces Increased Sustained In Vivo Protein Expression than Linear RNA.

Mice were injected and HEK293 cells were transfected with unmodified and mlW-modified human erythropoietin (hEpo) linear mRNAs and circRNAs. Equimolar transfection of mlW-mRNA and unmodified circRNA resulted in robust protein expression in HEK293 cells. hEpo linear mRNA and circRNA displayed similar relative protein expression patterns and cell viabilities in comparison to GLuc linear mRNA and circRNA upon equal weight transfection of HEK293 and A549 cells.

In mice, hEpo was detected in serum after the injection of hEpo circRNA or linear mRNA into visceral adipose. hEpo detected after the injection of unmodified circRNA decayed more slowly than that from unmodified or m1ψ-mRNA and was still present 42 hours post-injection. Serum hEpo rapidly declined upon the injection of unpurified circRNA splicing reactions or unmodified linear mRNA. Injection of unpurified splicing reactions produced a cytokine response detectable in serum that was not observed for the other RNAs, including purified circRNA.

### Example 7

### Circular RNA can be Effectively Delivered In Vivo or In Vitro Via Lipid Nanoparticles.

Purified circular RNA was formulated into lipid nanoparticles (LNPs) with the ionizable lipidoid cKK-E12 (Dong et al., 2014; Kauffman et al., 2015). The particles formed uniform multilamellar structures with an average size, polydispersity index, and encapsulation efficiency similar to that of particles containing commercially available control linear mRNA modified with 5moU.

Purified hEpo circRNA displayed greater expression than 5moU-mRNA when encapsulated in LNPs and added to HEK293 cells. Expression stability from LNP-RNA in HEK293 cells was similar to that of RNA delivered by transfection reagent, with the exception of a slight delay in decay for both 5moU-mRNA and circRNA. Both unmodified circRNA and 5moU-mRNA failed to activate RIG-I/IFN-β1 in vitro.

In mice, LNP-RNA was delivered by local injection into visceral adipose tissue or intravenous delivery to the liver. Serum hEpo expression from circRNA was lower but comparable with that from 5moU-mRNA 6 hours after delivery in both cases. Serum hEpo detected after adipose injection of unmodified LNP-circRNA decayed more slowly than that from LNP-5moU-mRNA, with a delay in expression decay present in serum that was similar to that noted in vitro, but serum hEpo after intravenous injection of LNP-circRNA or LNP-5moU-mRNA decayed at approximately the same rate. There was no increase in serum cytokines or local RIG-I, TNFα, or IL-6 transcript induction in any of these cases.

### Example 8

### Expression and Functional Stability by IRES in HEK293, HepG2, and ICIC7 Cells.

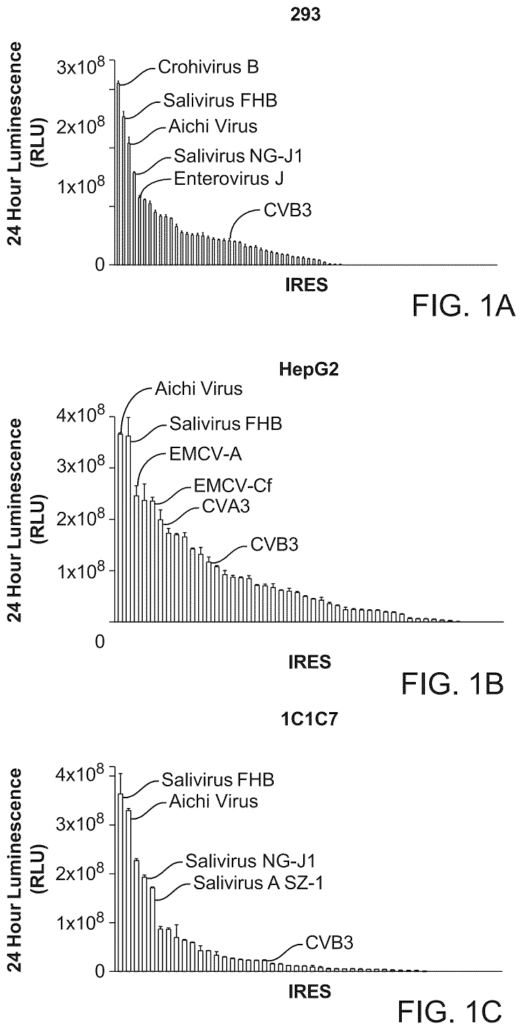

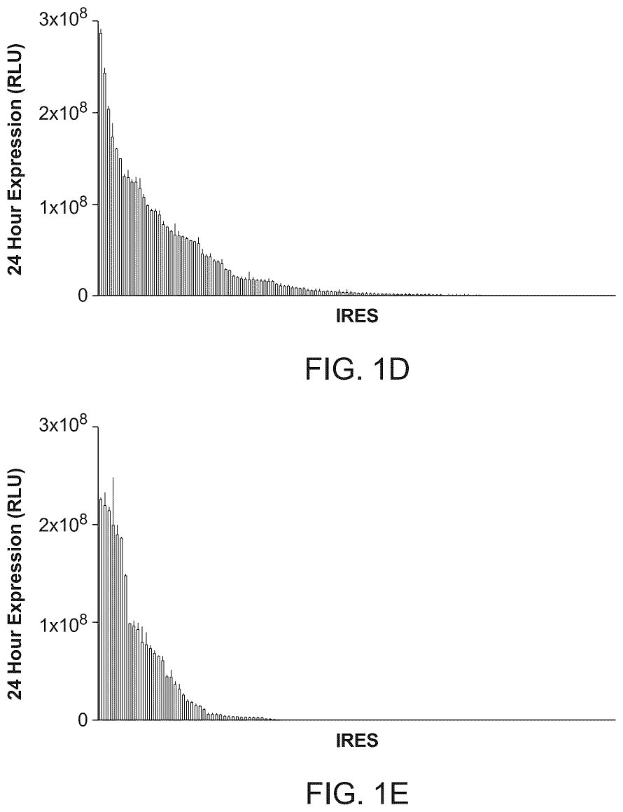

Constructs including anabaena intron/exon regions, a Gaussia luciferase expression sequence, and varying IRES were circularized. 100 ng of each circularization reaction was separately transfected into 20,000 HEK293 cells, HepG2 cells, and 1C1C7 cells using Lipofectamine MessengerMax. Luminescence in each supernatant was assessed after 24 hours as a measure of protein expression. In HEK293 cells, constructs including Crohivirus B, Salivirus FHB, Aichi Virus, Salivirus HG-J1, and Enterovirus J IRES produced the most luminescence at 24 hours (FIG. 1A). In HepG2 cells, constructs including Aichi Virus, Salivirus FHB, EMCV-Cf, and CVA3 IRES produced high luminescence at 24 hours (FIG. 1B). In 1C1C7 cells, constructs including Salivirus FHB, Aichi Virus, Salivirus NG-J1, and Salivirus A SZ-1 IRES produced high luminescence at 24 hours (FIG. 1C).

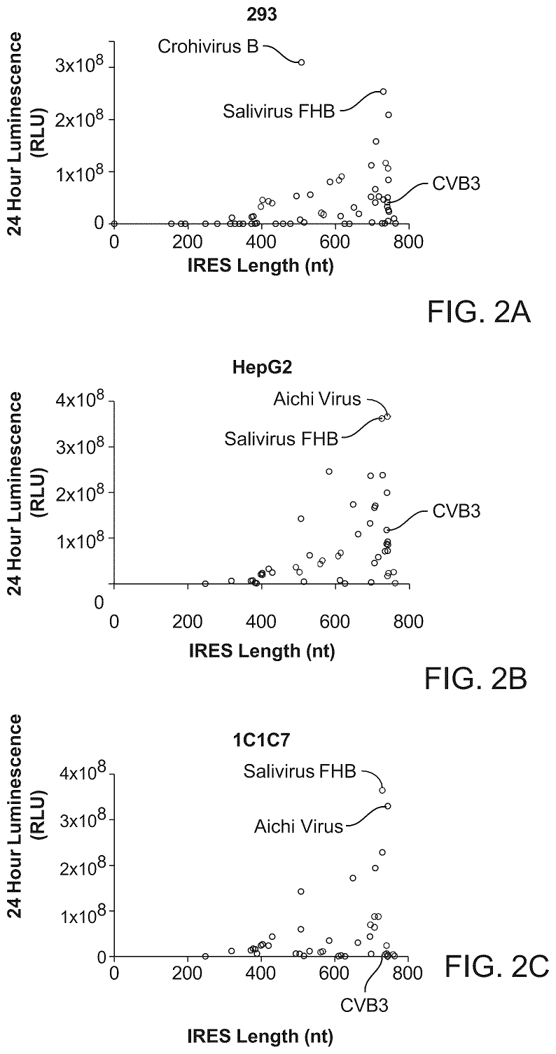

A trend of larger IRES producing greater luminescence at 24 hours was observed. Shorter total sequence length tends to increase circularization efficiency, so selecting a high expression and relatively short IRES may result in an improved construct. In HEK293 cells, a construct using the Crohivirus B IRES produced the highest luminescence, especially in comparison to other IRES of similar length (FIG. 2A). Expression from TRES constructs in HepG2 and 1C1C7 cells plotted against IRES size are in FIGS. 2B and 2C.

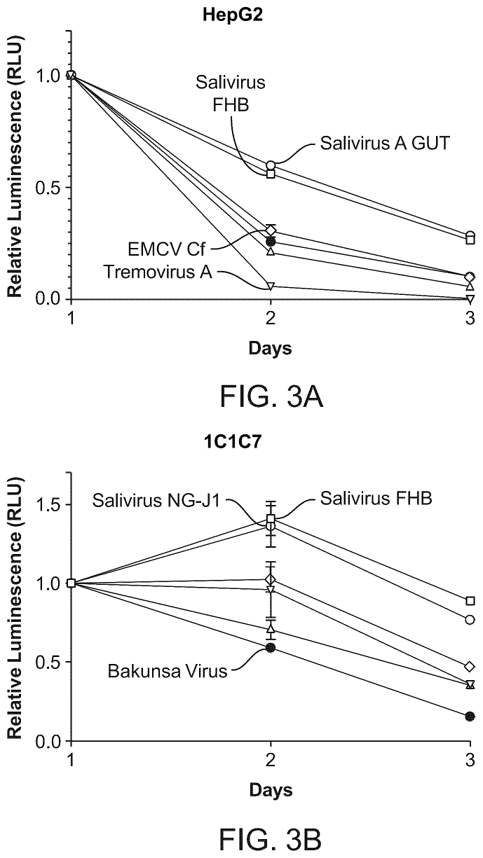

Functional stability of select IRES constructs in HepG2 and 1C1C7 cells were measured over 3 days. Luminescence from secreted Gaussia luciferase in supernatant was measured every 24 hours after transfection of 20,000 cells with 100 ng of each circularization reaction, followed by complete media replacement. Salivirus A GUT and Salivirus FHB exhibited the highest functional stability in HepG2 cells, and Salivirus N-J1 and Salivirus FHB produced the most stable expression in 1C1C7 cells (FIGS. 3A and 3B).

### Example 9

### Expression and Functional Stability by IRES in Jurkat Cells.

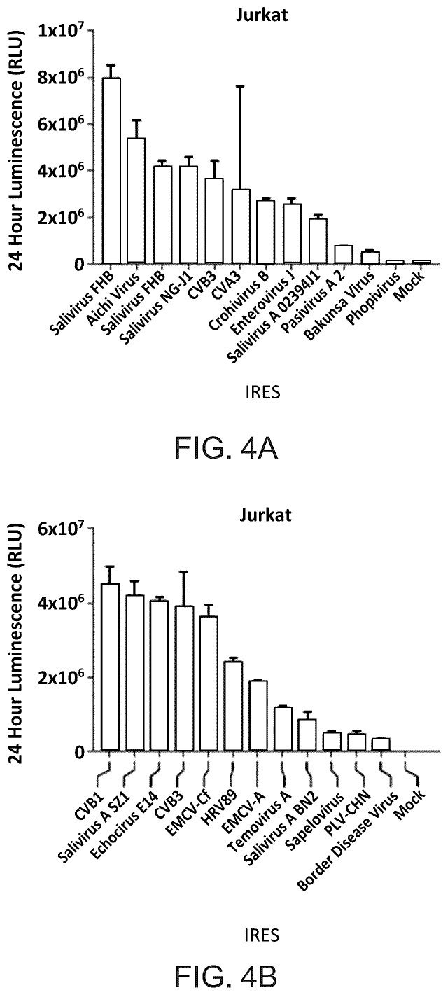

2 sets of constructs including anabaena intron/exon regions, a Gaussia luciferase expression sequence, and a subset of previously tested IRES were circularized. 60,000 Jurkat cells were electroporated with 1 μg of each circularization reaction. Luminescence from secreted Gaussia luciferase in supernatant was measured 24 hours after electroporation. A CVB3 IRES construct was included in both sets for comparison between sets and to previously defined IRES efficacy. CVB1 and Salivirus A SZ1 IRES constructs produced the most expression at 24 h. Data can be found in FIGS. 4A and 4B.

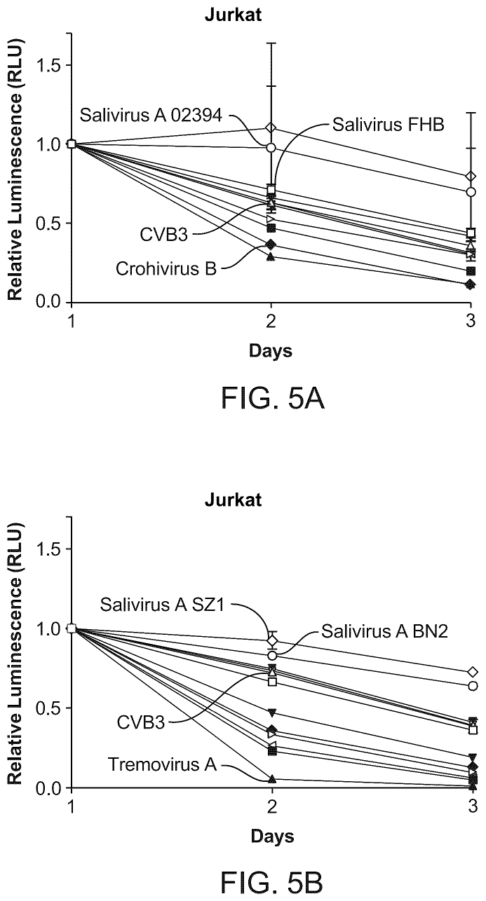

Functional stability of the TRES constructs in each round of electroporated Jurkat cells was measured over 3 days. Luminescence from secreted Gaussia luciferase in supernatant was measured every 24 hours after electroporation of 60,000 cells with 1 μg of each circularization reaction, followed by complete media replacement (FIGS. 5A and 5B).

Salivirus A SZ1 and Salivirus A BN2 IRES constructs had high functional stability compared to other constructs.

### Example 10

### Expression, Functional Stability, and Cytokine Release of Circular and Linear RNA in Jurkat Cells.

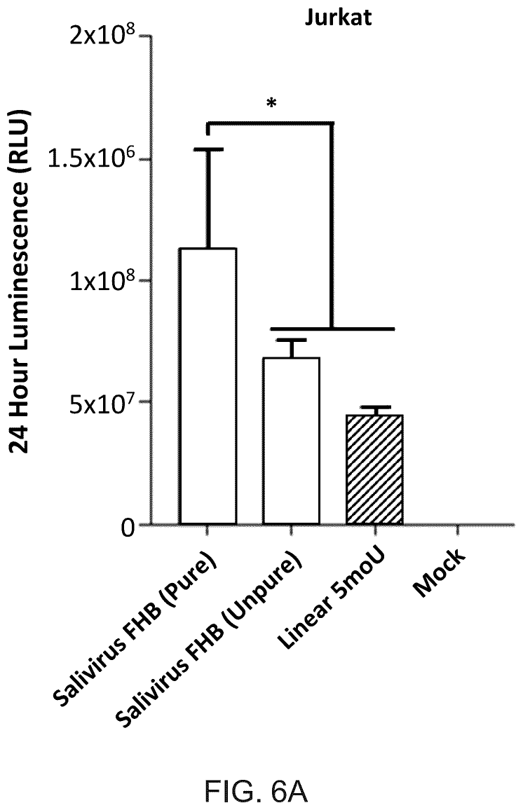

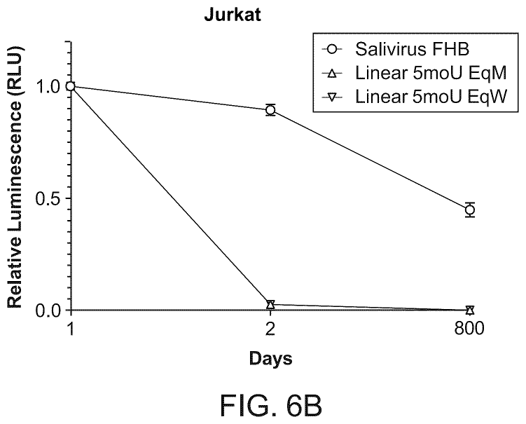

A construct including anabaena intron/exon regions, a Gaussia luciferase expression sequence, and a Salivirus FHB TRES was circularized. mRNA including a Gaussia luciferase expression sequence and a ˜150 nt polyA tail (e.g., SEQ ID NO: 3279), and modified to replace 100% of uridine with 5-methoxy uridine (5moU) is commercially available and was purchased from Trilink. 5moU nucleotide modifications have been shown to improve mRNA stability and expression (Bioconjug Chem. 2016 Mar. 16; 27(3):849-53). Expression of modified mRNA, circularization reactions (unpure), and circRNA purified by size exclusion HPLC (pure) in Jurkat cells were measured and compared (FIG. 6A). Luminescence from secreted Gaussia luciferase in supernatant was measured 24 hours after electroporation of 60,000 cells with 1 μg of each RNA species. Luminescence from secreted Gaussia luciferase in supernatant was measured every 24 hours after electroporation of 60,000 cells with 1 ug of each RNA species, followed by complete media replacement. A comparison of functional stability data of modified mRNA and circRNA in Jurkat cells over 3 days is in FIG. 6B.

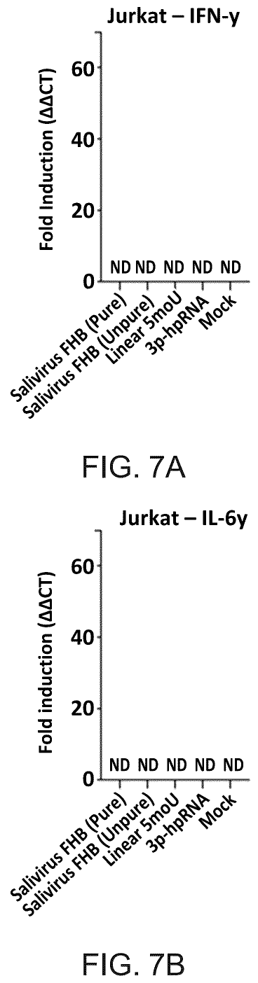

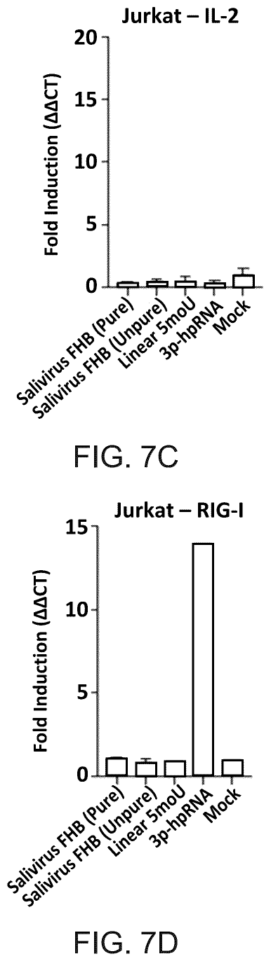

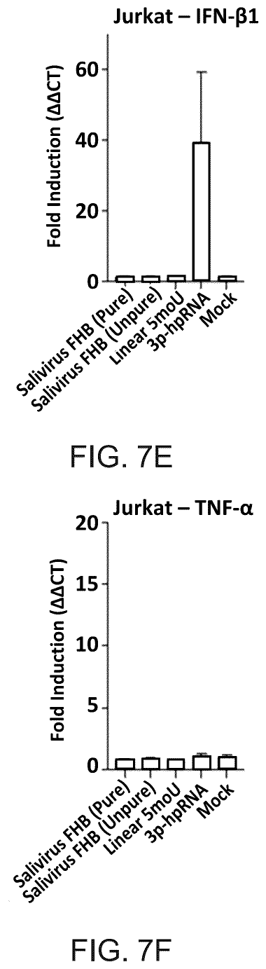

IFNγ (FIG. 7A), IL-6 (FIG. 7B), IL-2 (FIG. 7C), RIG-I (FIG. 7D), IFN-β 1 (FIG. 7E), and TNFα (FIG. 7F) transcript induction was measured 18 hours after electroporation of 60,000 Jurkat cells with 1 μg of each RNA species described above and 3p-hpRNA (5′ triphosphate hairpin RNA, which is a known RIG-I agonist).

### Example 11

### Expression of Circular and Linear RNA in Monocytes and Macrophages.

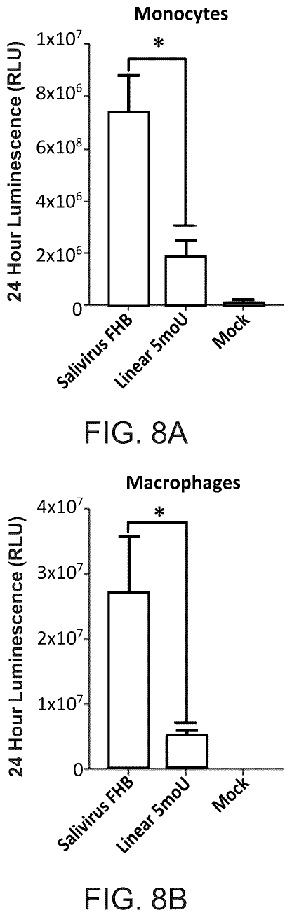

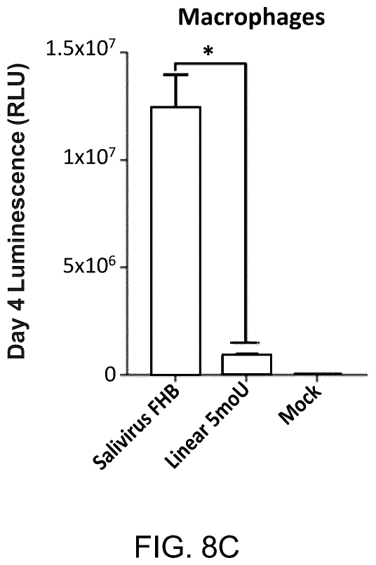

A construct including anabaena intron/exon regions, a Gaussia luciferase expression sequence, and a Salivirus FHB TRES was circularized. mRNA including a Gaussia luciferase expression sequence and a ˜150 nt polyA tail (e.g., SEQ ID NO: 3279), and modified to replace 100% of uridine with 5-methoxy uridine (5moU) was purchased from Trilink. Expression of circular and modified mRNA was measured in human primary monocytes (FIG. 8A) and human primary macrophages (FIG. 8B). Luminescence from secreted Gaussia luciferase in supernatant was measured 24 hours after electroporation of 60,000 cells with 1 μg of each RNA species. Luminescence was also measured 4 days after electroporation of human primary macrophages with media changes every 24 hours (FIG. 8C). The difference in luminescence was statistically significant in each case (p<0.05).

### Example 12

### Expression and Functional Stability by IRES in Primary T Cells.

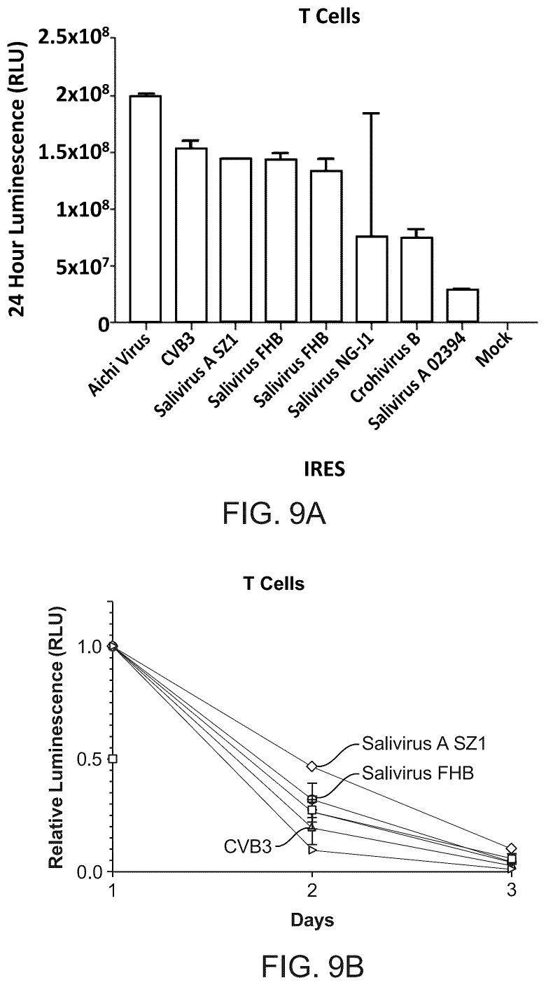

Constructs including anabaena intron/exon regions, a Gaussia luciferase expression sequence, and a subset of previously tested IRES were circularized and reaction products were purified by size exclusion HPLC. 150,000 primary human CD3+ T-cells were electroporated with 1 μg of each circRNA. Luminescence from secreted Gaussia luciferase in supernatant was measured 24 hours after electroporation (FIG. 9A). Aichi Virus and CVB3 IRES constructs had the most expression at 24 hours.

Luminescence was also measured every 24 hours after electroporation for 3 days in order to compare functional stability of each construct (FIG. 9B). The construct with a Salivirus A SZ1 TRES was the most stable.

### Example 13

### Expression and Functional Stability of Circular and Linear RNA in Primary T Cells and PBMCs.

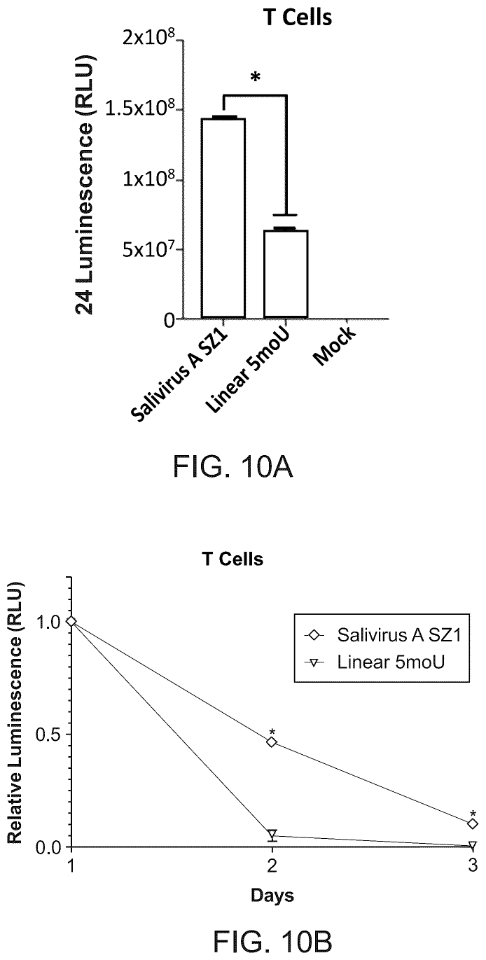

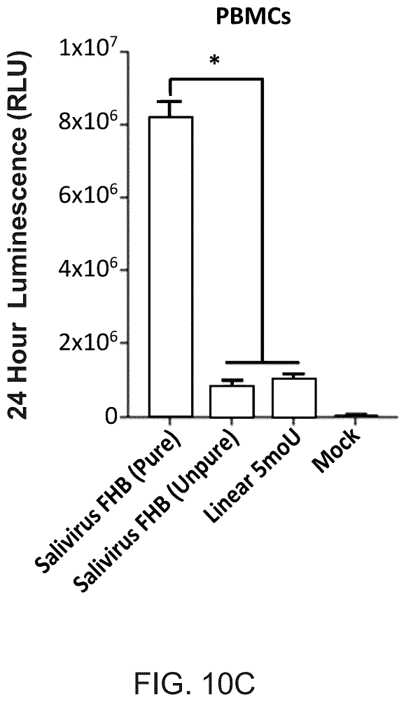

Constructs including anabaena intron/exon regions, a Gaussia luciferase expression sequence, and a Salivirus A SZ1 IRES or Salivirus FHB IRES were circularized. mRNA including a Gaussia luciferase expression sequence and a ˜150 nt polyA tail (e.g., SEQ ID NO: 3279), and modified to replace 100% of uridine with 5-methoxy uridine (5moU) and was purchased from Trilink. Expression of Salivirus A SZ1 IRES HPLC purified circular and modified mRNA was measured in human primary CD3+ T cells. Expression of Salivirus FHB HPLC purified circular, unpurified circular and modified mRNA was measured in human PBMCs. Luminescence from secreted Gaussia luciferase in supernatant was measured 24 hours after electroporation of 150,000 cells with 1 μg of each RNA species. Data for primary human T cells is in FIGS. 10A and 10B, and data for PBMCs is in FIG. 10C. The difference in expression between the purified circular RNA and unpurified circular RNA or linear RNA was significant in each case (p<0.05).

Luminescence from secreted Gaussia luciferase in primary T cell supernatant was measured every 24 hours after electroporation over 3 days in order to compare construct functional stability. Data is shown in FIG. 10B. The difference in relative luminescence from the day 1 measurement between purified circular RNA and linear RNA was significant at both day 2 and day 3 for primary T cells.

### Example 14

### Circularization Efficiency by Permutation Site in Anabaena Intron.

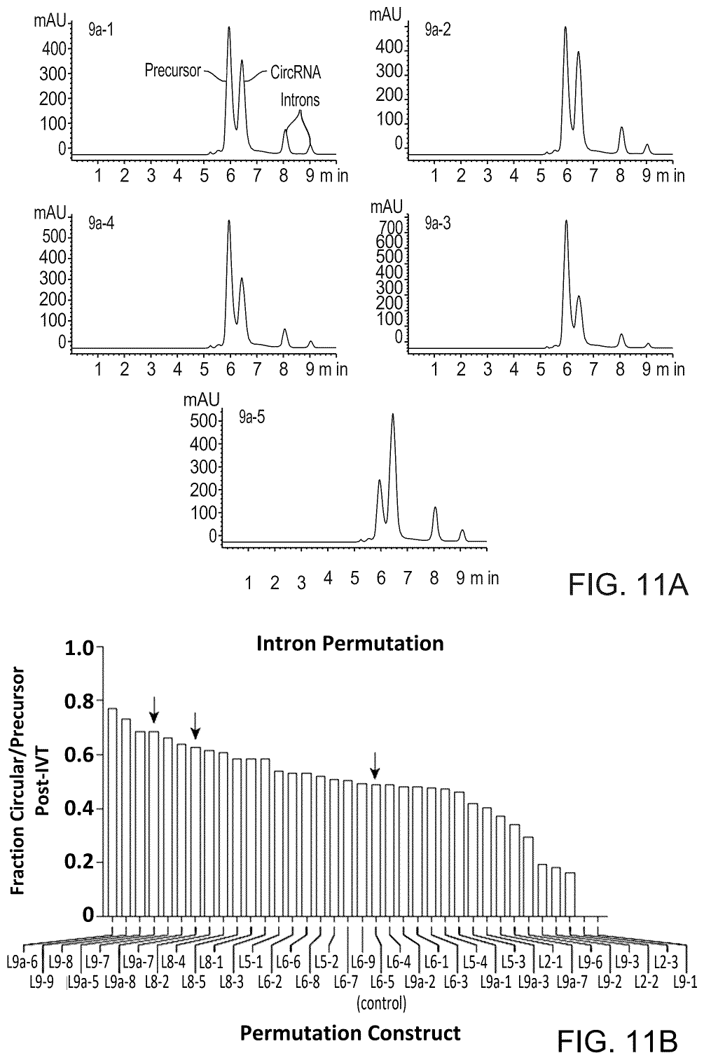

RNA constructs including a CVB3 IRES, a Gaussia luciferase expression sequence, anabaena intron/exon regions, spacers, internal duplex regions, and homology arms were produced. Circularization efficiency of constructs using the traditional anabaena intron permutation site and 5 consecutive permutations sites in P9 was measured by HPLC. HPLC chromatograms for the 5 consecutive permutation sites in P9 are shown in FIG. 11A.

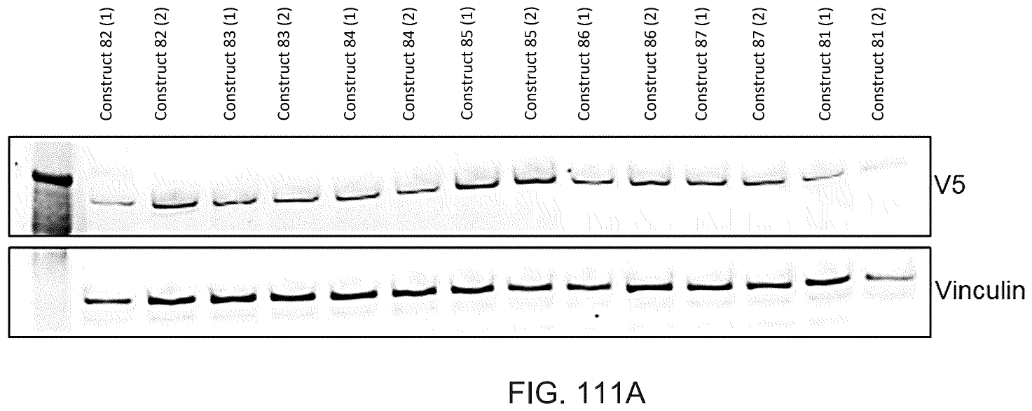

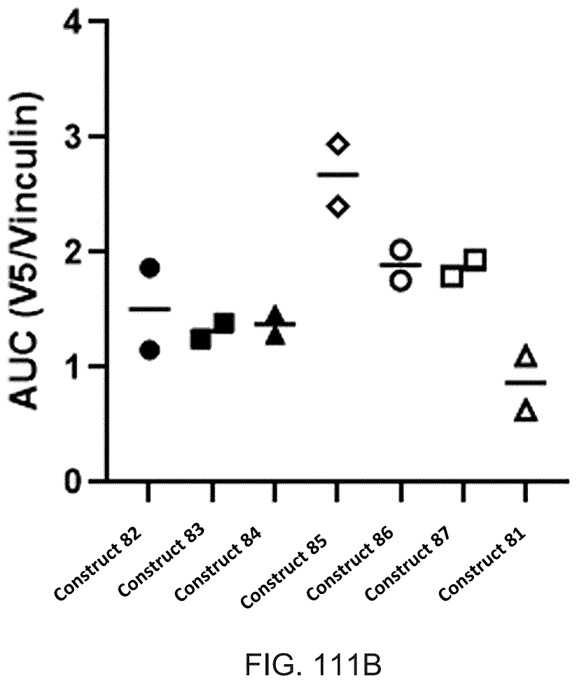

Circularization efficiency was measured at a variety of permutation sites. Circularization efficiency is defined as the area under the HPLC chromatogram curve for each of: circRNA/(circRNA+precursor RNA). Ranked quantification of circularization efficiency at each permutation site is in FIG. 11B. 3 permutation sites (indicated in FIG. 111B) were selected for further investigation.

Circular RNA in this example was circularized by in vitro transcription (IVT) then purified via spin column. Circularization efficiency for all constructs would likely be higher if the additional step of incubation with Mg²⁺ and guanosine nucleotide were included; however, removing this step allowed for comparison between, and optimization of, circular RNA constructs. This level of optimization is especially useful for maintaining high circularization efficiency with large RNA constructs, such as those encoding chimeric antigen receptors.

### Example 15

### Circularization Efficiency of Alternative Introns.

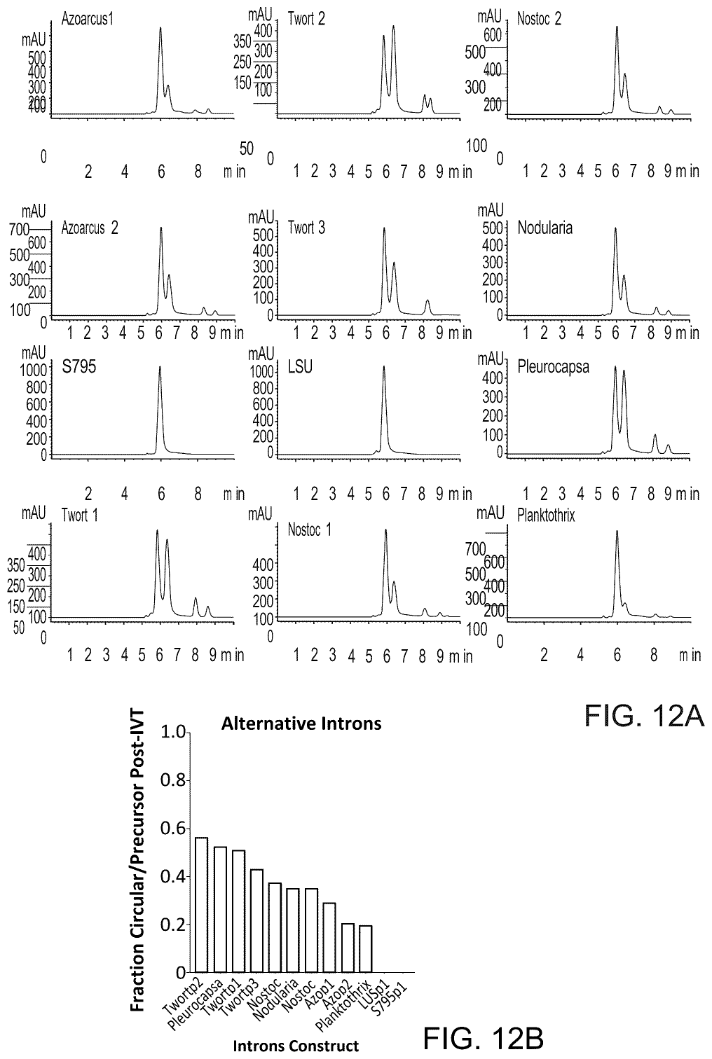

Precursor RNA containing a permuted group 1 intron of variable species origin or permutation site and several constant elements including: a CVB3 IRES, a Gaussia luciferase expression sequence, spacers, internal duplex regions, and homology arms were created. Circularization data can be found in FIG. 12. FIG. 12A shows chromatograms resolving precursor, CircRNA and introns. FIG. 12B provides ranked quantification of circularization efficiency, based on the chromatograms shown in FIG. 12A, as a function of intron construct.

Circular RNA in this example was circularized by in vitro transcription (IVT) then spin column purification. Circularization efficiency for all constructs would likely be higher if the additional step of incubation with Mg²⁺ and guanosine nucleotide were included; however, removing this step allows for comparison between, and optimization of, circular RNA constructs. This level of optimization is especially useful for maintaining high circularization efficiency with large RNA constructs, such as those encoding chimeric antigen receptors.

### Example 16

### Circularization Efficiency by Homology Arm Presence or Length.

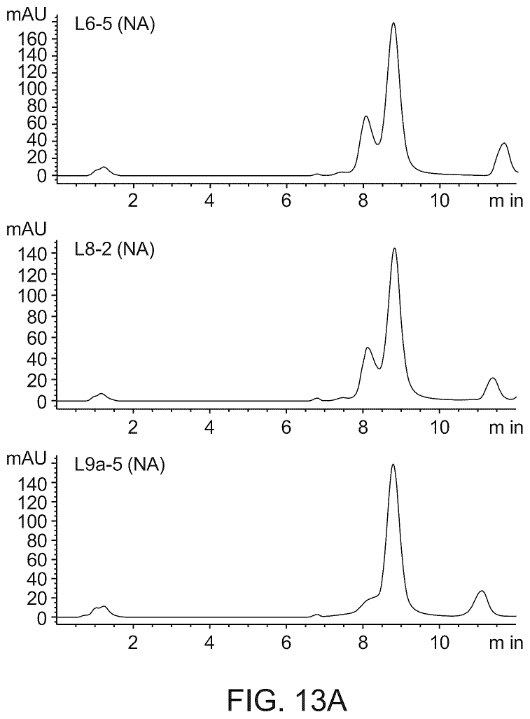

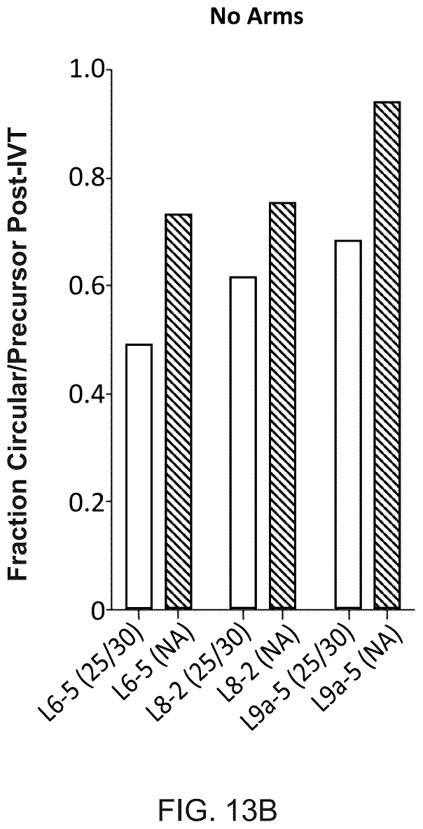

RNA constructs including a CVB3 IRES, a Gaussia luciferase expression sequence, anabaena intron/exon regions, spacers, and internal duplex regions were produced. Constructs representing 3 anabaena intron permutation sites were tested with 30 nt, 25% GC homology arms or without homology arms (“NA”). These constructs were allowed to circularize without the step of incubation with Mg²⁺. Circularization efficiency was measured and compared. Data can be found in FIG. 13. Circularization efficiency was higher for each construct lacking homology arms. FIG. 13A provides ranked quantification of circularization efficiency; FIG. 13B provides chromatograms resolving precursor, circRNA and introns.

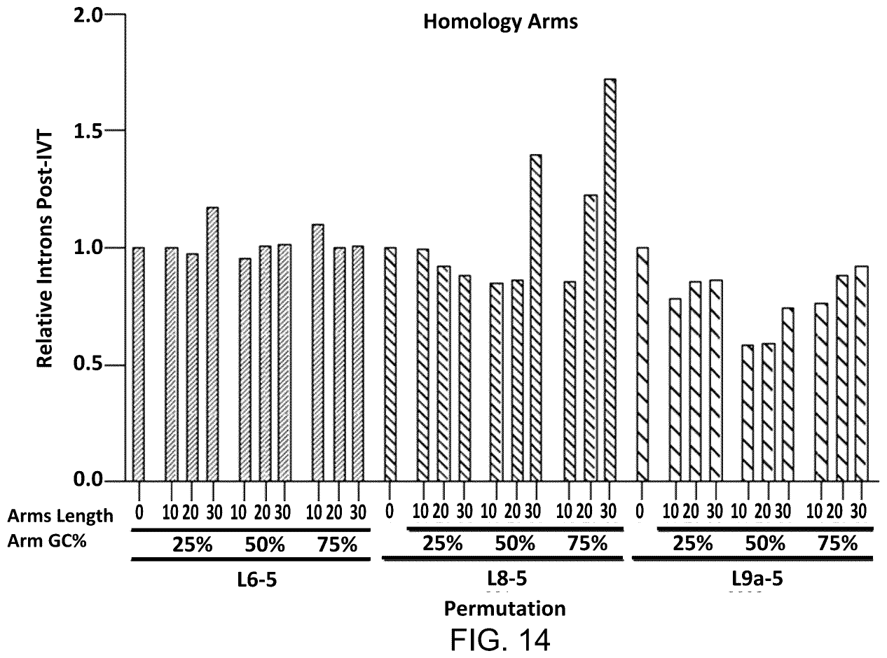

For each of the 3 permutation sites, constructs were created with 10 nt, 20 nt, and 30 nt arm length and 25%, 50%, and 75% GC. Splicing efficiency of these constructs was measured and compared to constructs without homology arms (FIG. 14). Splicing efficiency is defined as the proportion of free introns relative to the total RNA in the splicing reaction.

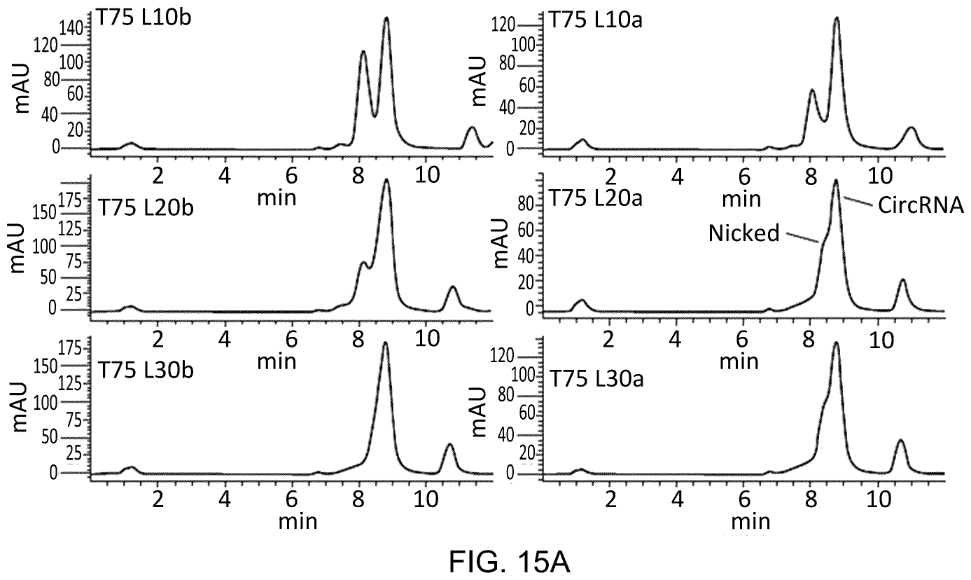

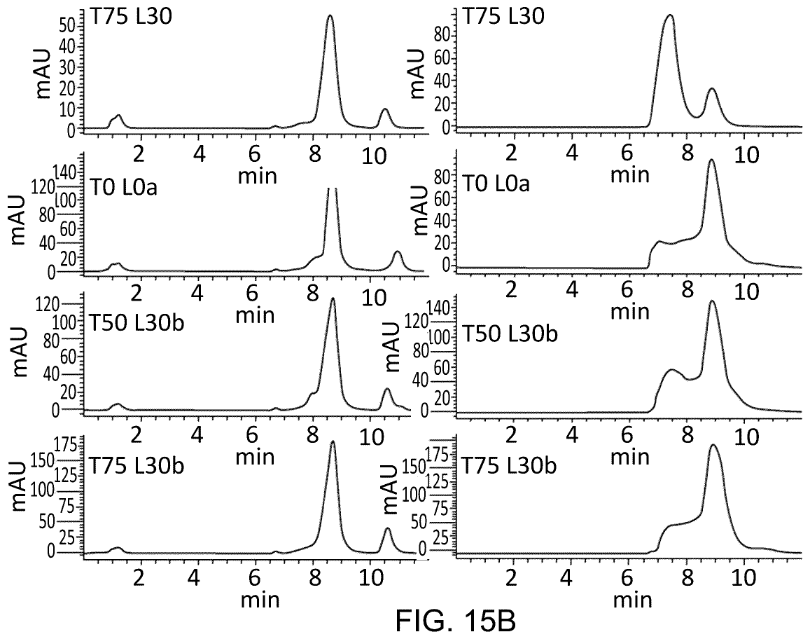

FIG. 15A (left) contains HPLC chromatograms showing the contribution of strong homology arms to improved splicing efficiency. Top left: 75% GC content, 10 nt homology arms. Center left: 75% GC content, 20 nt homology arms. Bottom left: 75% GC content, 30 nt homology arms.

FIG. 15A (right) shows HPLC chromatograms indicating increased splicing efficiency paired with increased nicking, appearing as a shoulder on the circRNA peak. Top right: 75% GC content, 10 nt homology arms. Center right: 75% GC content, 20 nt homology arms. Bottom right: 75% GC content, 30 nt homology arms.

FIG. 15B (left) shows select combinations of permutation sites and homology arms hypothesized to demonstrate improved circularization efficiency.

FIG. 15B (right) shows select combinations of permutation sites and homology arms hypothesized to demonstrate improved circularization efficiency, treated with E. coli polyA polymerase.

Circular RNA in this example was circularized by in vitro transcription (IVT) then spin-column purified. Circularization efficiency for all constructs would likely be higher if an additional Mg²⁺ incubation step with guanosine nucleotide were included; however, removing this step allowed for comparison between, and optimization of, circular RNA constructs. This level of optimization is especially useful for maintaining high circularization efficiency with large RNA constructs, such as those encoding chimeric antigen receptors.

### Example 17

### Circular RNA Encoding Chimeric Antigen Receptors.

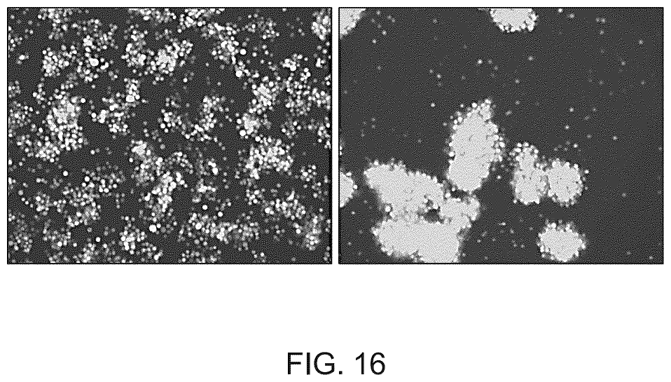

Constructs including anabaena intron/exon regions, a Kymriah chimeric antigen receptor (CAR) expression sequence, and a CVB3 IRES were circularized. 100,000 human primary CD3+ T cells were electroporated with 500 ng of circRNA and co-cultured for 24 hours with Raji cells stably expressing GFP and firefly luciferase. Effector to target ratio (E:T ratio) 0.75:1. 100,000 human primary CD3+ T cells were mock electroporated and co-cultured as a control (FIG. 16).

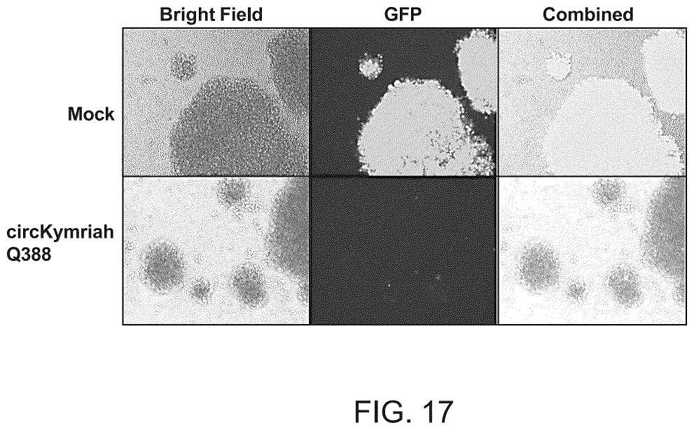

Sets of 100,000 human primary CD3+ T cells were mock electroporated or electroporated with 1 μg of circRNA then co-cultured for 48 hours with Raji cells stably expressing GFP and firefly luciferase. E:T ratio 10:1 (FIG. 17).

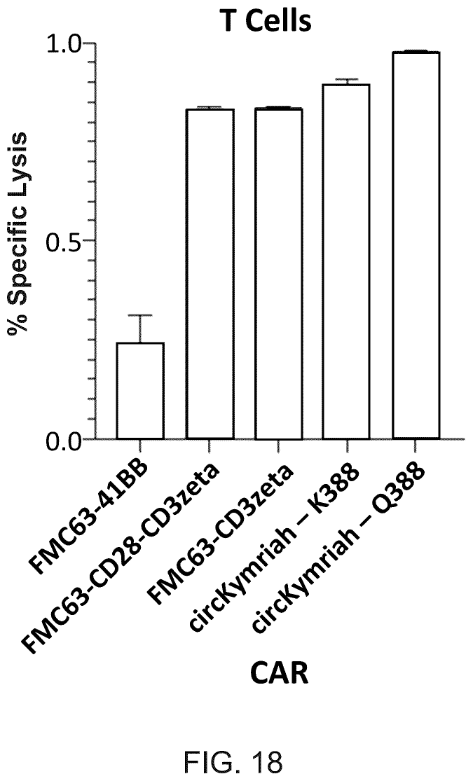

Quantification of specific lysis of Raji target cells was determined by detection of firefly luminescence (FIG. 18). 100,000 human primary CD3+ T cells either mock electroporated or electroporated with circRNA encoding different CAR sequences were co-cultured for 48 hours with Raji cells stably expressing GFP and firefly luciferase. % Specific lysis defined as 1-[CAR condition luminescence]/[mock condition luminescence]. E:T ratio 10:1.

### Example 18

### Expression and Functional Stability of Circular and Linear RNA in Jurkat Cells and Resting Human T Cells.

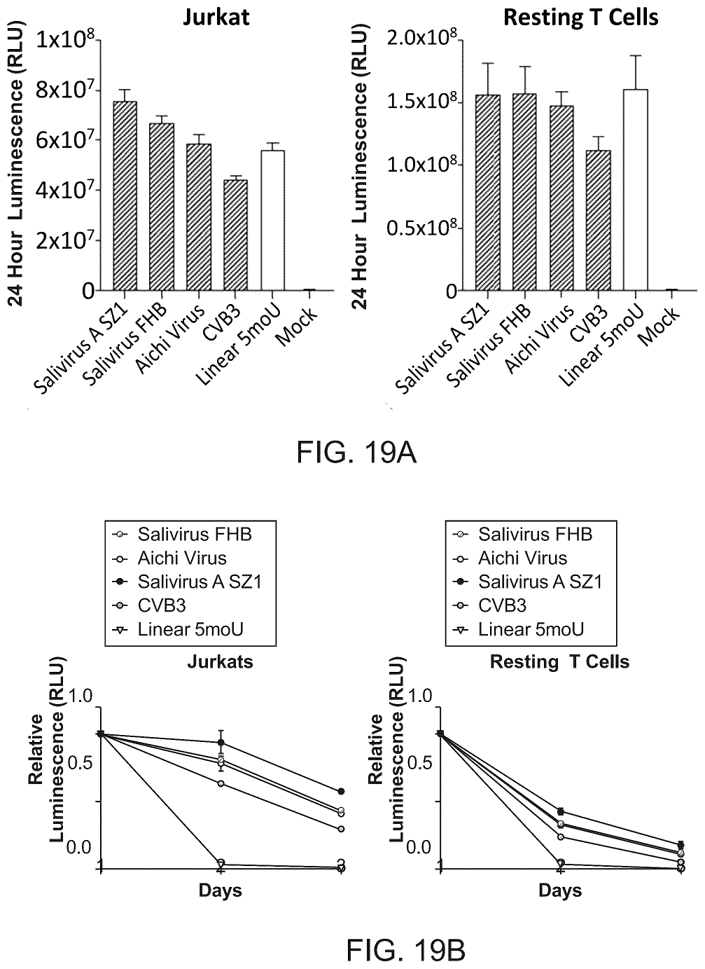

Constructs including anabaena intron/exon regions, a Gaussia luciferase expression sequence, and a subset of previously tested IRES were circularized and reaction products were purified by size exclusion HPLC. 150,000 Jurkat cells were electroporated with 1 μg of circular RNA or 5moU-mRNA. Luminescence from secreted Gaussia luciferase in supernatant was measured 24 hours after electroporation (FIG. 19A left). 150,000 resting primary human CD3+ T cells (10 days post-stimulation) were electroporated with 1 μg of circular RNA or 5moU-mRNA. Luminescence from secreted Gaussia luciferase in supernatant was measured 24 hours after electroporation (FIG. 19A right).

Luminescence from secreted Gaussia luciferase in supernatant was measured every 24 hours after electroporation, followed by complete media replacement. Functional stability data is shown in FIG. 19B. Circular RNA had more functional stability than linear RNA in each case, with a more pronounced difference in Jurkat cells.

### Example 19

IFN-β1, RIG-I, IL-2, IL-6, IFNγ, and TNFα Transcript Induction of Cells Electroporated with Linear RNA or Varying Circular RNA Constructs.

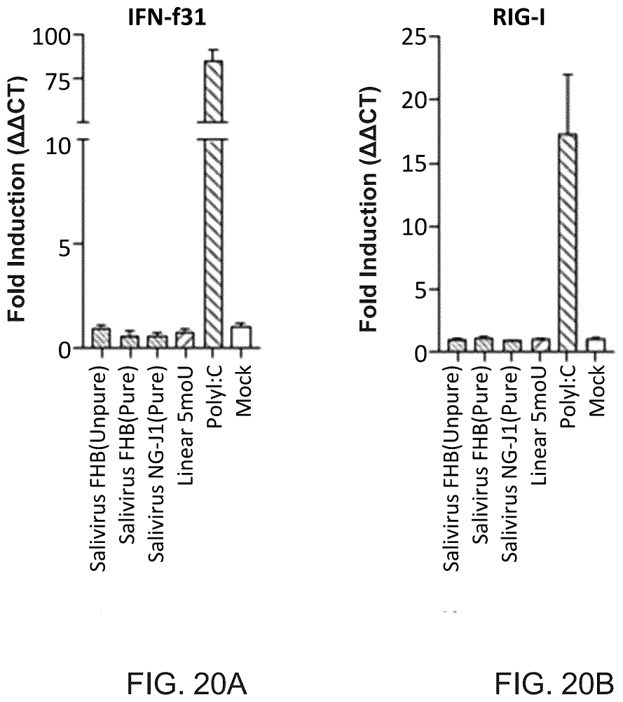

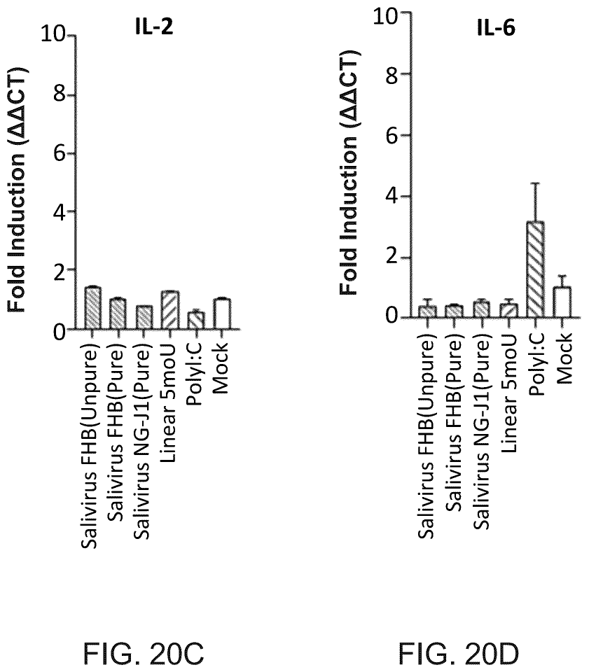

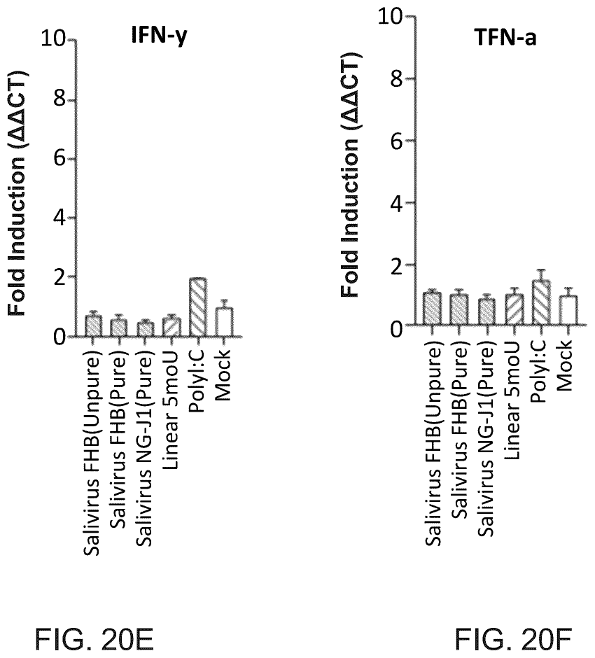

Constructs including anabaena intron/exon regions, a Gaussia luciferase expression sequence, and a subset of previously tested IRES were circularized and reaction products were purified by size exclusion HPLC. 150,000 CD3+ human T cells were electroporated with 1 μg of circular RNA, 5moU-mRNA, or immunostimulatory positive control poly inosine:cytosine. IFN-β1 (FIG. 20A), RIG-I (FIG. 20B), IL-2 (FIG. 20C), IL-6 (FIG. 20D), IFN-7 (FIG. 20E), and TNF-α (FIG. 20F) transcript induction was measured 18 hours after electroporation.

### Example 20

Specific lysis of target cells and IFNγ transcript induction by CAR expressing cells electroporated with different amounts of circular or linear RNA; specific lysis of target and non-target cells by CAR expressing cells at different E: T ratios.

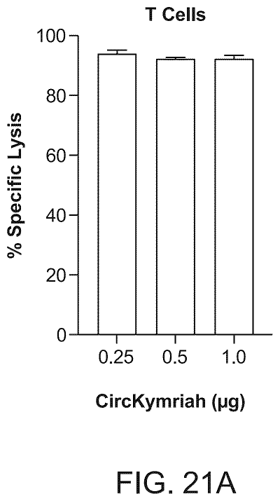

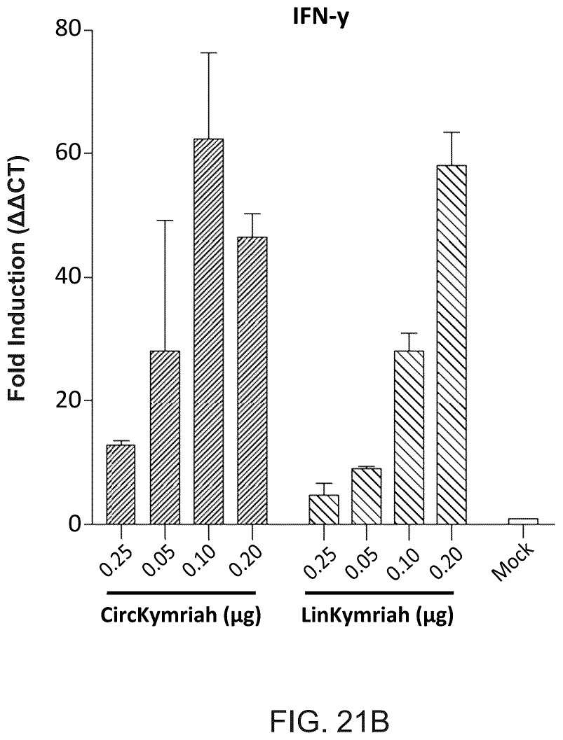

Constructs including anabaena intron/exon regions, an anti-CD19 CAR expression sequence, and a CVB3 IRES were circularized and reaction products were purified by size exclusion HPLC. 150,000 human primary CD3+ T cells either mock electroporated or electroporated with different quantities of circRNA encoding an anti-CD19 CAR sequence were co-cultured for 12 hours with Raji cells stably expressing GFP and firefly luciferase at an E:T ratio of 2:1. Specific lysis of Raji target cells was determined by detection of firefly luminescence (FIG. 21A). % Specific lysis was defined as 1-[CAR condition luminescence]/[mock condition luminescence]. IFNγ transcript induction was measured 24 hours after electroporation (FIG. 21).

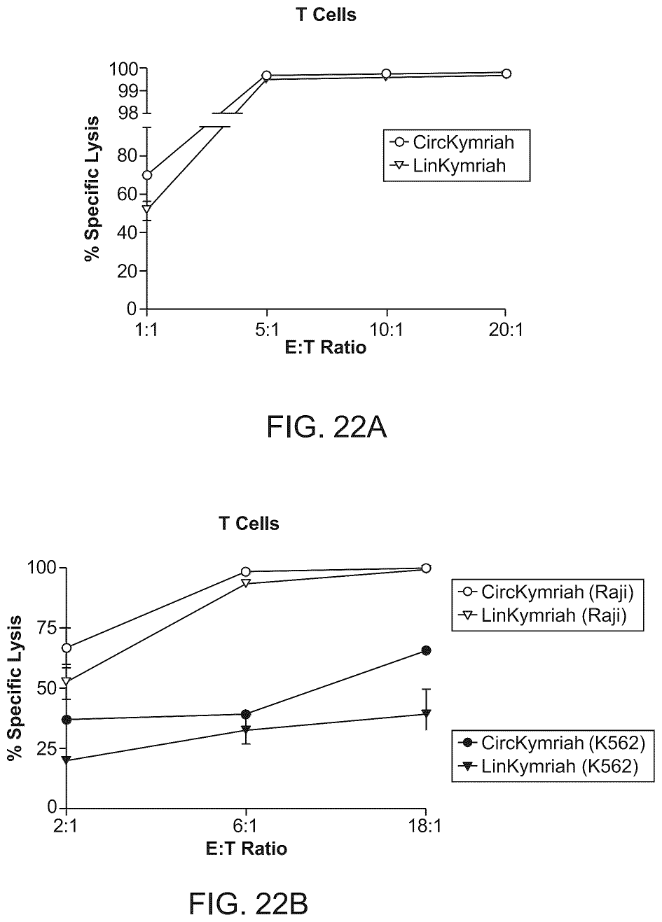

150,000 human primary CD3+ T cells were either mock electroporated or electroporated with 500 ng circRNA or mlW-mRNA encoding an anti-CD19 CAR sequence, then co-cultured for 24 hours with Raji cells stably expressing firefly luciferase at different E:T ratios. Specific lysis of Raji target cells was determined by detection of firefly luminescence (FIG. 22A). Specific lysis was defined as 1-[CAR condition luminescence]/[mock condition luminescence].

CAR expressing T cells were also co-cultured for 24 hours with Raji or K562 cells stably expressing firefly luciferase at different E:T ratios. Specific lysis of Raji target cells or K562 non-target cells was determined by detection of firefly luminescence (FIG. 22B). % Specific lysis is defined as 1-[CAR condition luminescence]/[mock condition luminescence].

### Example 21

Specific Lysis of Target Cells by T Cells Electroporated with Circular RNA or Linear RNA Encoding a CAR.

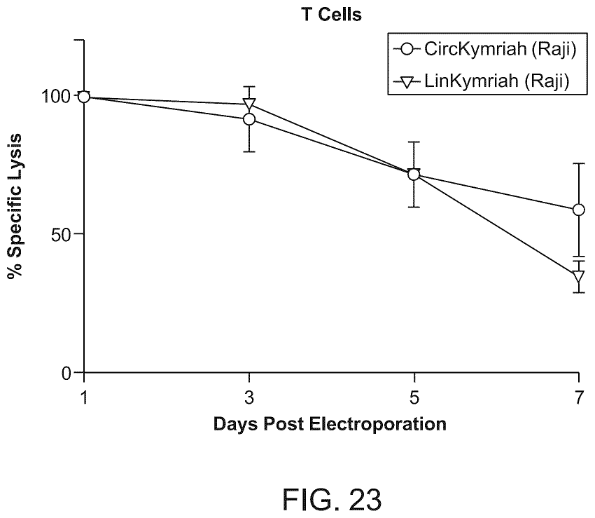

Constructs including anabaena intron/exon regions, an anti-CD19 CAR expression sequence, and a CVB3 IRES were circularized and reaction products were purified by size exclusion HPLC. Human primary CD3+ T cells were electroporated with 500 ng of circular RNA or an equimolar quantity of m1ψ-mRNA, each encoding a CD19-targeted CAR. Raji cells were added to CAR-T cell cultures over 7 days at an E:T ratio of 10:1. % Specific lysis was measured for both constructs at 1, 3, 5, and 7 days (FIG. 23).

### Example 22

### Specific Lysis of Raji Cells by T Cells Expressing an Anti-CD19 CAR or an Anti-BCMA CAR.

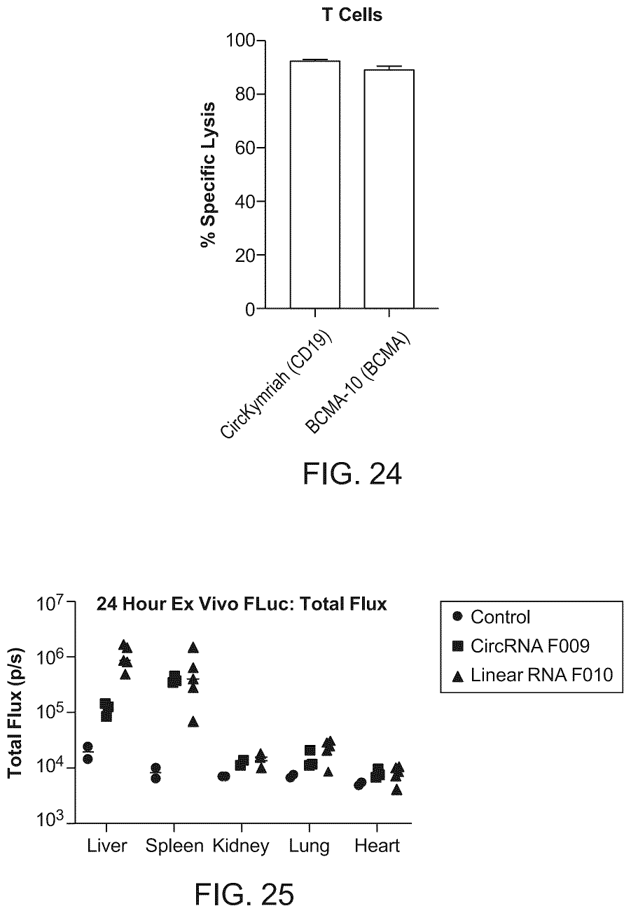

Constructs including anabaena intron/exon regions, anti-CD19 or anti-BCMA CAR expression sequence, and a CVB3 IRES were circularized and reaction products were purified by size exclusion HPLC. 150,000 primary human CD3+ T cells were electroporated with 500 ng of circRNA, then were co-cultured with Raji cells at an E:T ratio of 2:1. % Specific lysis was measured 12 hours after electroporation (FIG. 24).

### Example 23

### Expression, Functional Stability, and Cytokine Transcript Induction of Circular and Linear RNA Expressing Antigens.

Constructs including one or more antigen expression sequences are circularized and reaction products are purified by size exclusion HPLC. Antigen presenting cells are electroporated with circular RNA or mRNA.

In vitro antigen production is measured via ELISA. Optionally, antigen production is measured every 24 hours after electroporation. Cytokine transcript induction or release is measured 18 hours after electroporation of antigen presenting cells with circular or linear RNA encoding antigens. The tested cytokines may include IFN-β1, RIG-I, IL-2, TL-6, IFNγ, RANTES, and TNFα.

In vitro antigen production and cytokine induction are measured using purified circRNA, purified circRNA plus antisense circRNA, and unpurified circRNA in order to find the ratio that best preserves expression and immune stimulation.

### Example 24

### In Vivo Antigen and Antibody Expression in Animal Models.

To assess the ability of antigen encoding circRNAs to facilitate antigen expression and antibody production in vivo, escalating doses of RNA encoding one or more antigens is introduced into mice via intramuscular injection.

Mice are injected once, blood collected after 28 days, then injected again, with blood collected 14 days thereafter. Neutralizing antibodies against antigen of interest is measured via ELISA.

### Example 25

### Protection Against Infection.

To assess the ability of antigen encoding circRNAs to protect against or cure an infection, RNA encoding one or more antigens of a virus (such as influenza) is introduced into mice via intramuscular injection.

Mice receive an initial injection and boost injections of circRNA encoding one or more antigens. Protection from a virus such as influenza is determined by weight loss and mortality over 2 weeks.

### Example 26

### Protein Expression by Organ

Circular or linear RNA encoding FLuc was generated and loaded into transfer vehicles with the following formulation: 50% ionizable Lipid 10b-15 represented by

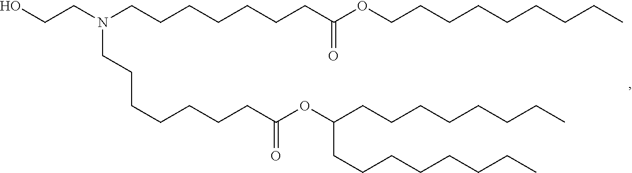 [SMILES: CCCCCCCCCOC(=O)CCCCCCCN(CCO)CCCCCCCC(=O)OC(CCCCCCCC)CCCCCCCC]

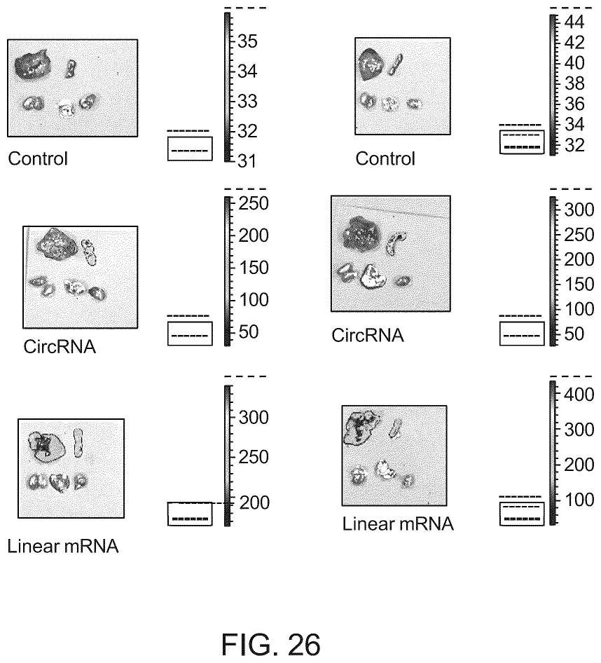

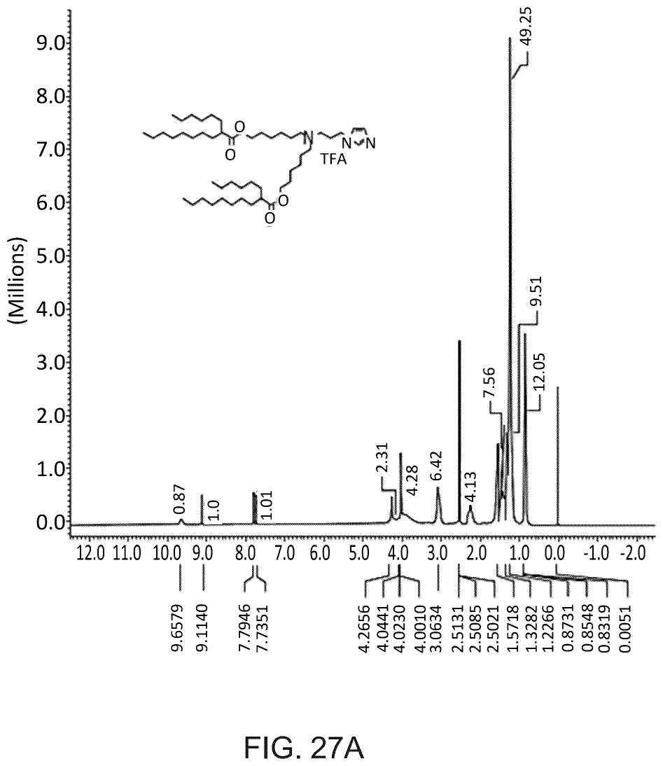

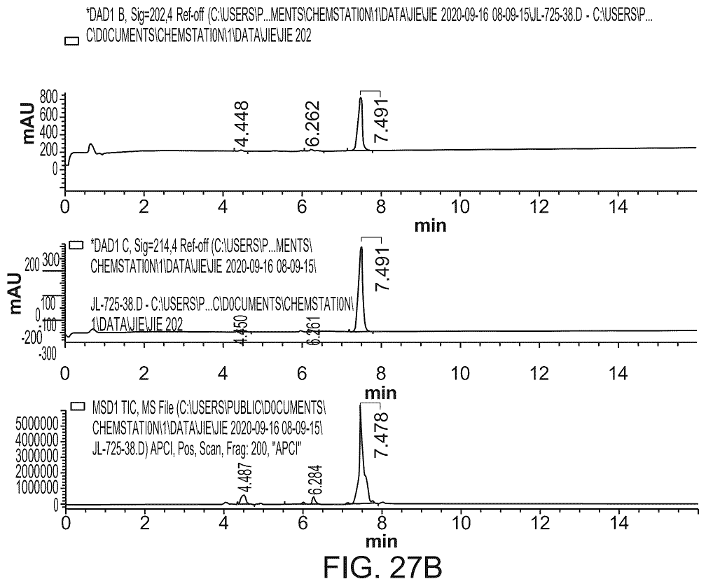

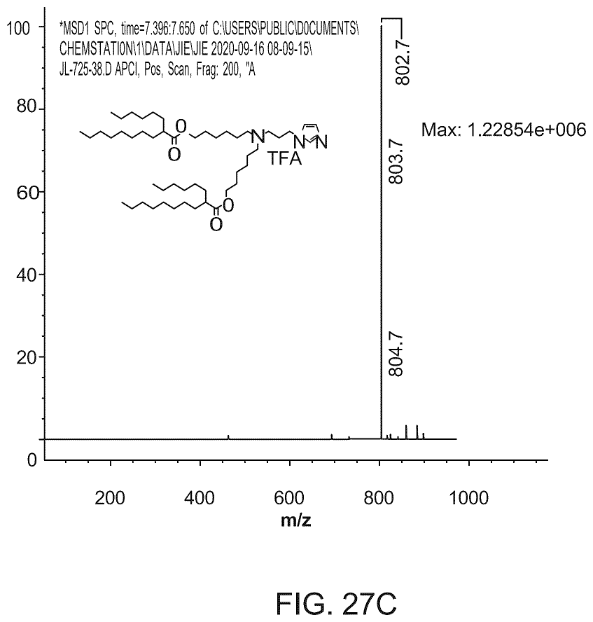

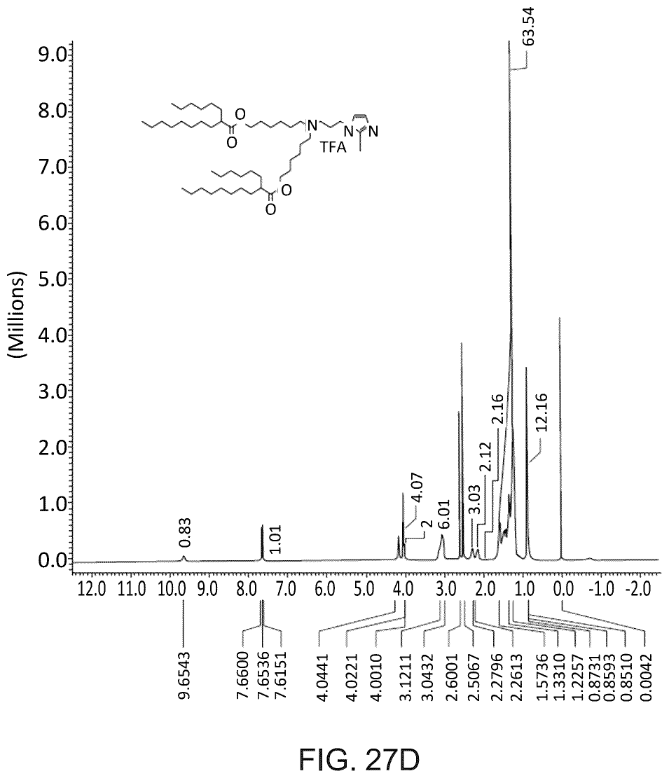

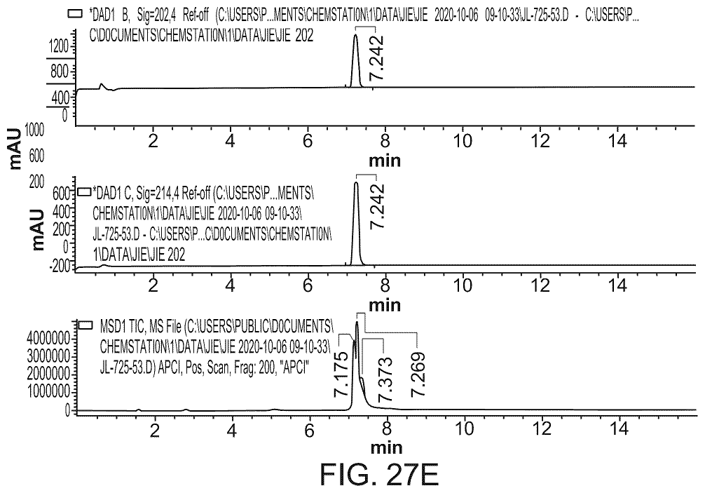

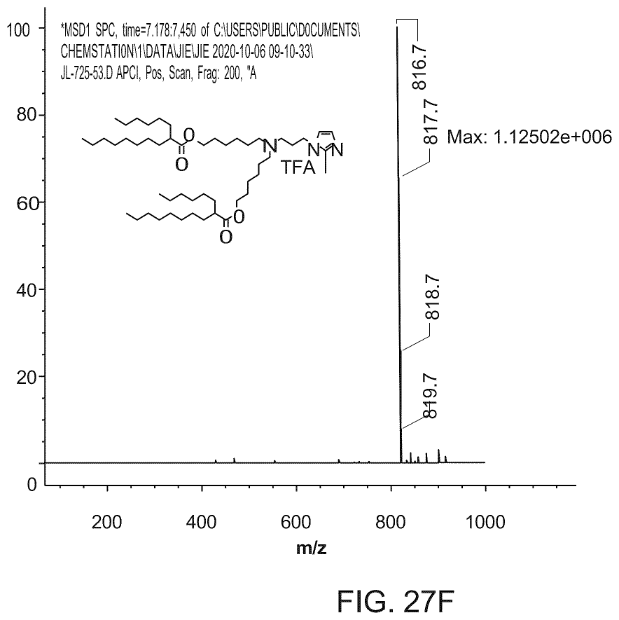

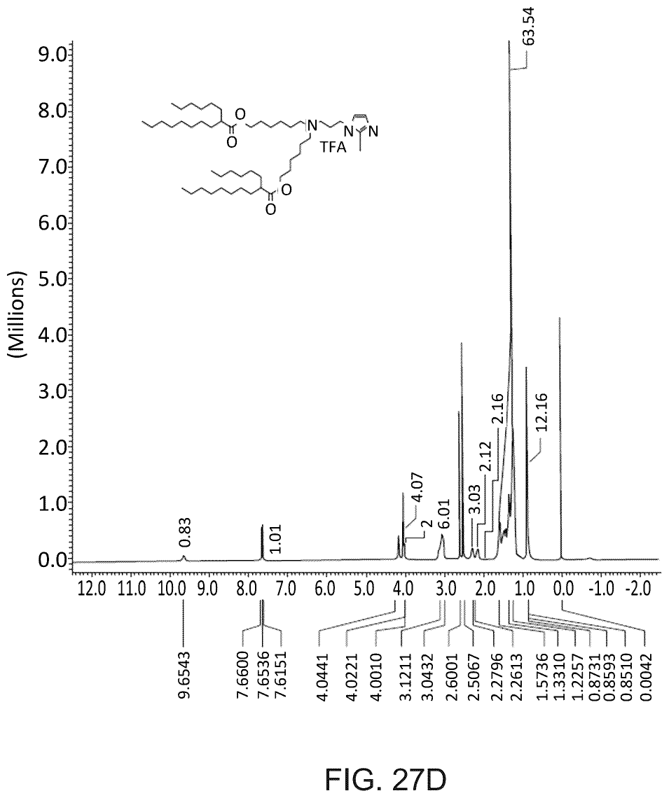

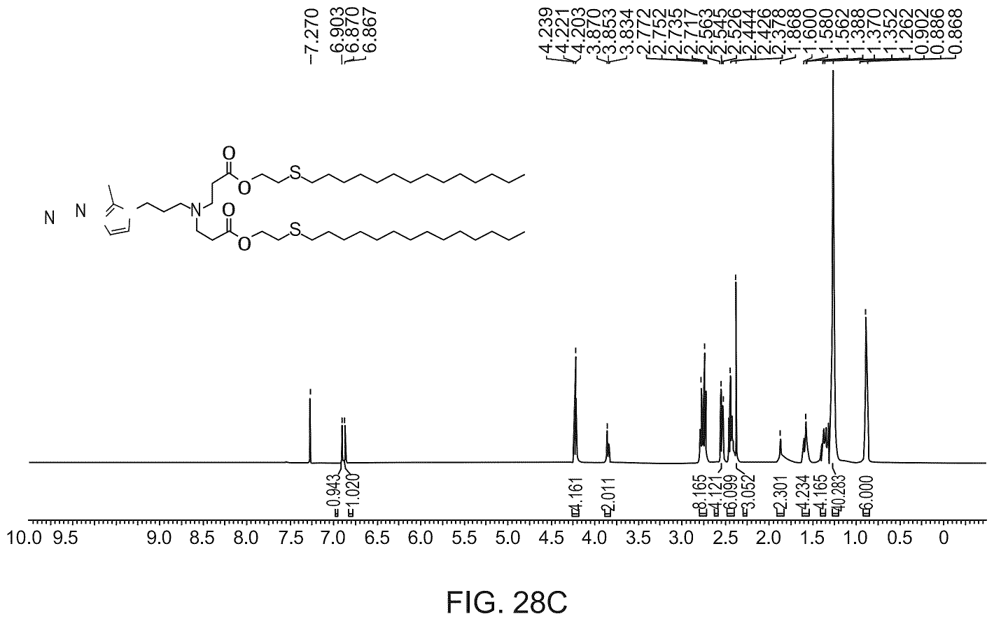

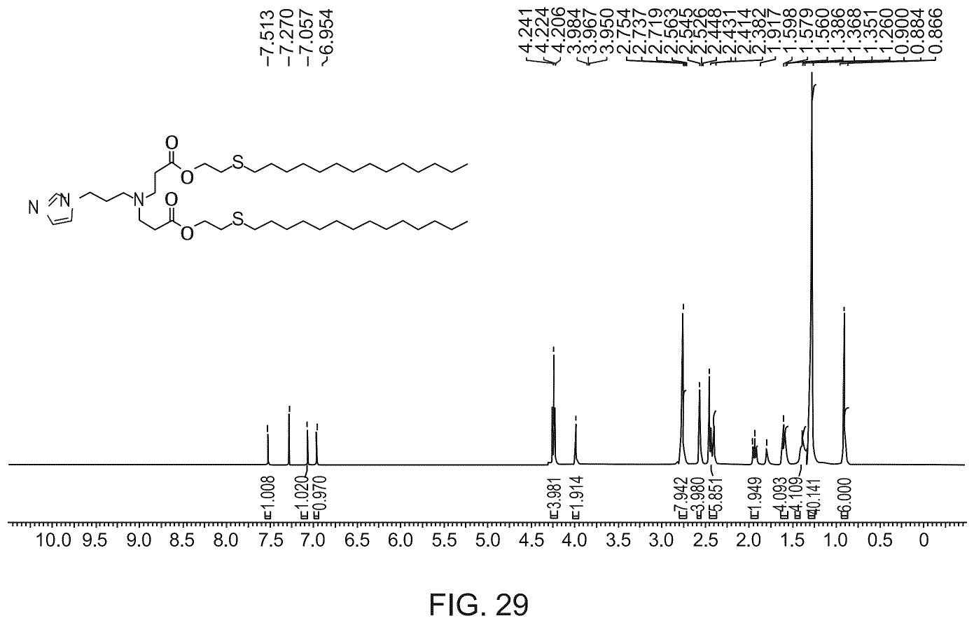

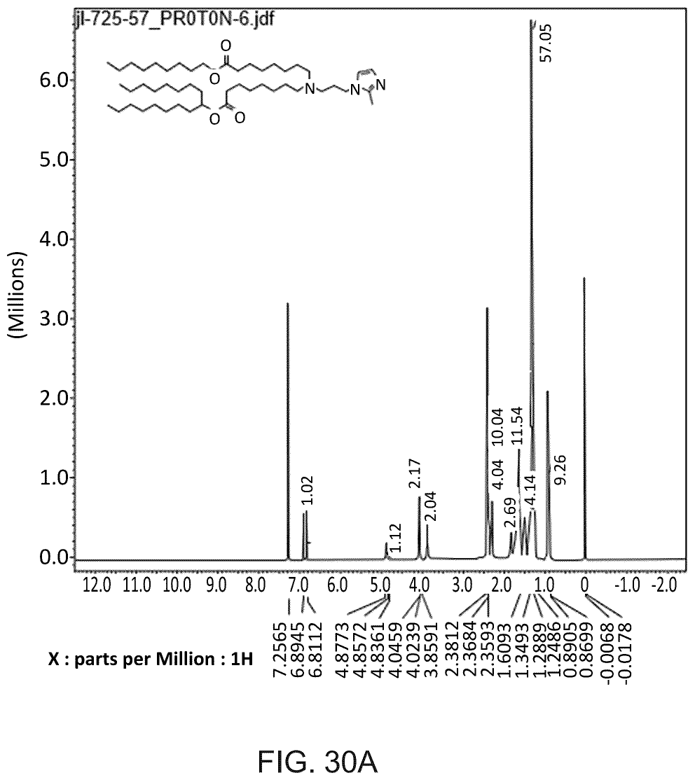

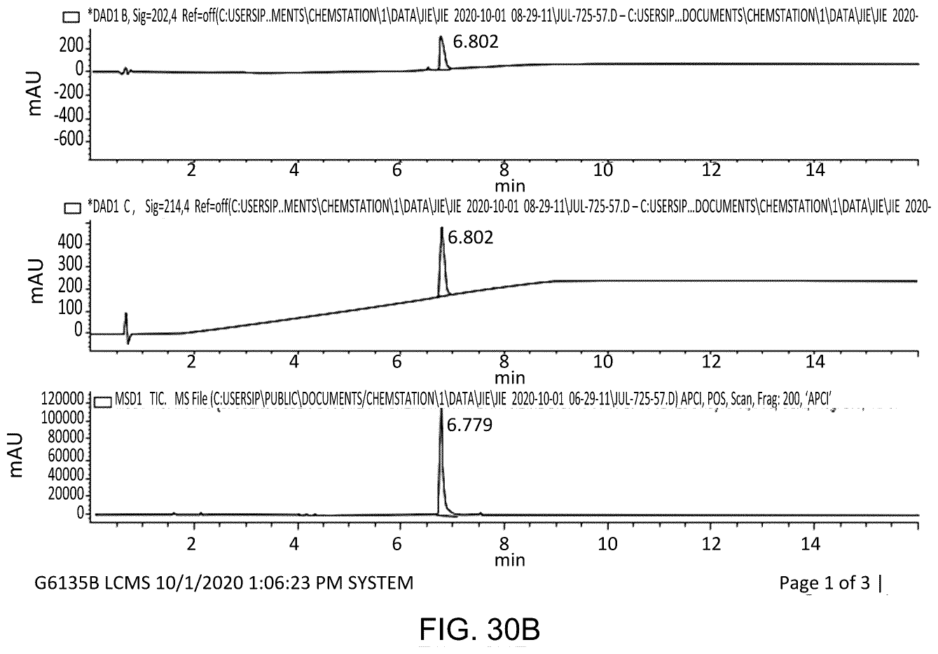

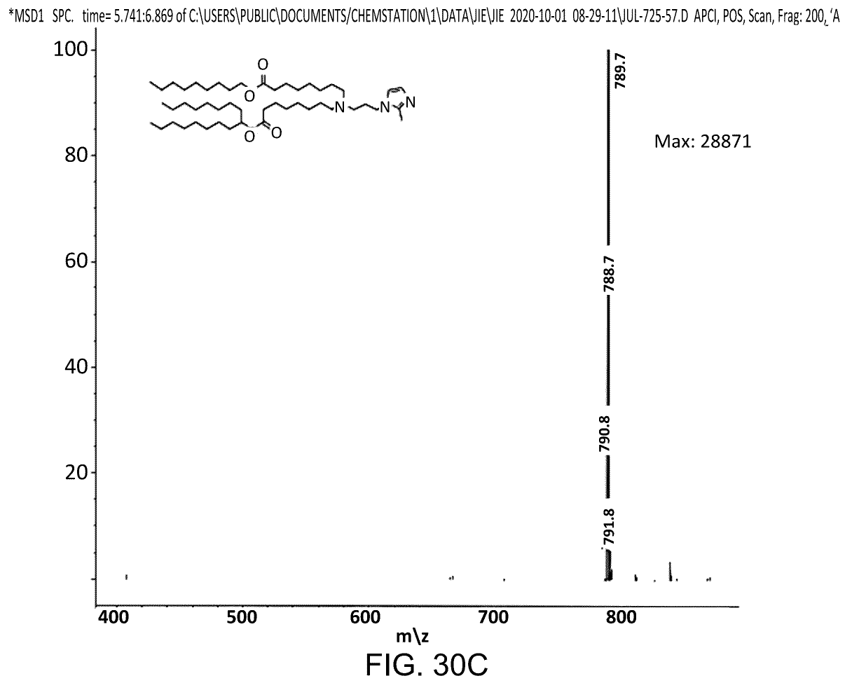

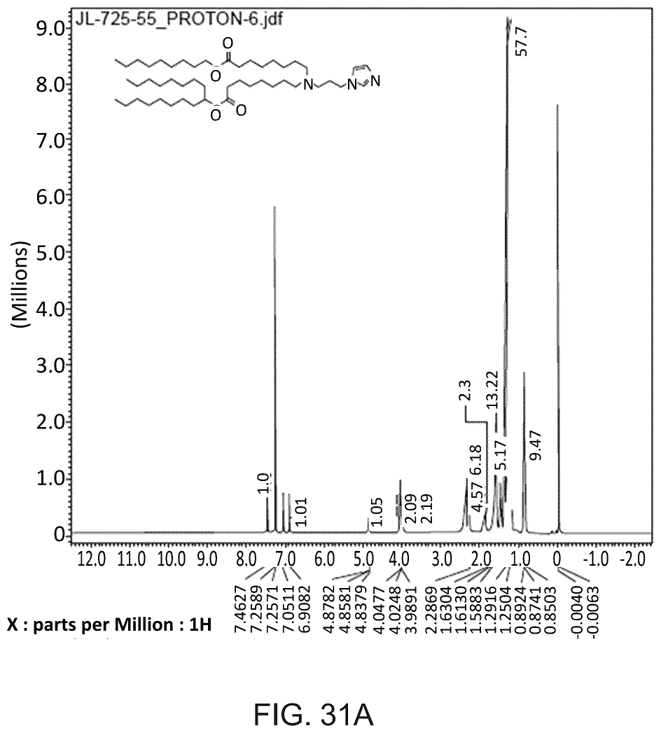

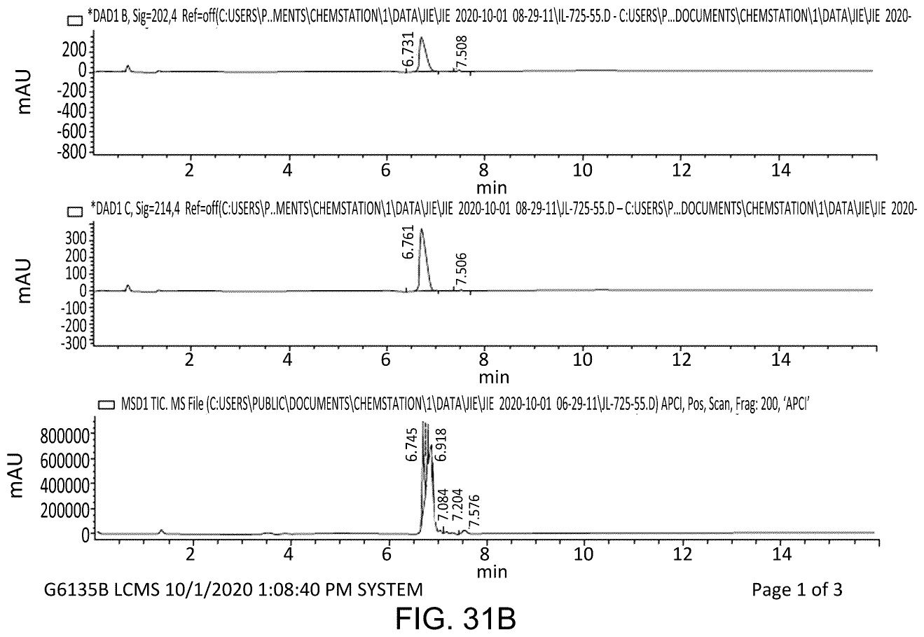

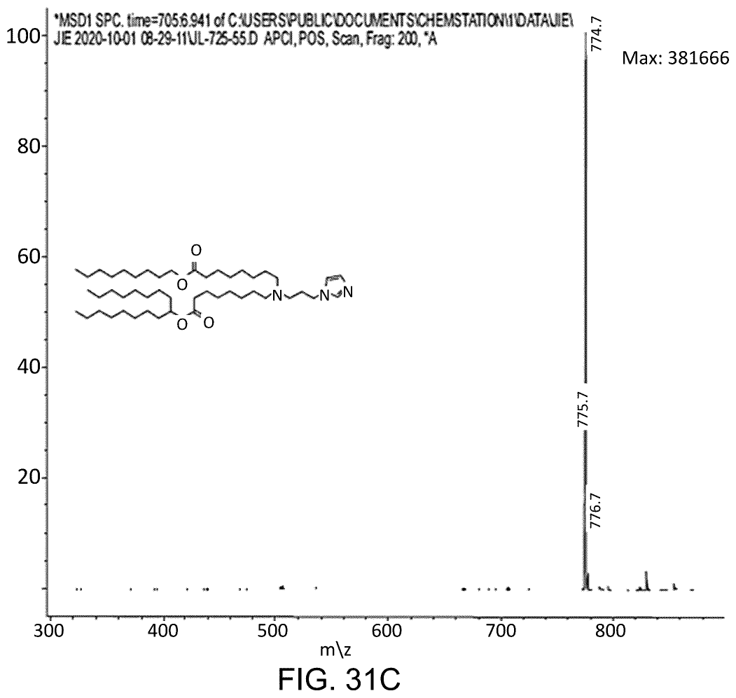

10% DSPC, 1.5% PEG-DMG, 38.5% cholesterol. CD-1 mice were dosed at 0.2 mg/kg and luminescence was measured at 6 hours (live IVIS) and 24 hours (live IVIS and ex vivo IVIS). Total Flux (photons/second over a region of interest) of the liver, spleen, kidney, lung, and heart was measured (FIGS. 25 and 26).

### Example 27

### Distribution of Expression in the Spleen

Circular or linear RNA encoding GFP is generated and loaded into transfer vehicles with the following formulation: 50% ionizable Lipid 10b-15 represented by

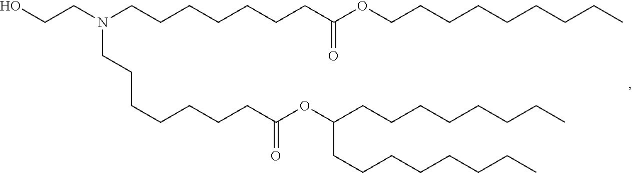 [SMILES: CCCCCCCCCOC(=O)CCCCCCCN(CCO)CCCCCCCC(=O)OC(CCCCCCCC)CCCCCCCC]

10% DSPC, 1.5% PEG-DMG, 38.5% cholesterol. The formulation is administered to CD-1 mice. Flow cytometry is run on spleen cells to determine the distribution of expression across cell types.

### Example 28

### Example 28a: Production of Nanoparticle Compositions

In order to investigate safe and efficacious nanoparticle compositions for use in the delivery of circular RNA to cells, a range of formulations are prepared and tested. Specifically, the particular elements and ratios thereof in the lipid component of nanoparticle compositions are optimized.

Nanoparticles can be made in a 1 fluid stream or with mixing processes such as microfluidics and T-junction mixing of two fluid streams, one of which contains the circular RNA and the other has the lipid components.

Lipid compositions are prepared by combining an ionizable lipid, optionally a helper lipid (such as DOPE, DSPC, or oleic acid obtainable from Avanti Polar Lipids, Alabaster, AL), a PEG lipid (such as 1,2-dimyristoyl-sn-glycerol methoxypolyethylene glycol, also known as PEG-DMG, obtainable from Avanti Polar Lipids, Alabaster, AL), and a structural lipid such as cholesterol at concentrations of about, e.g., 40 or 50 mM in a solvent, e.g., ethanol. Solutions should be refrigerated for storage at, for example, −20° C. Lipids are combined to yield desired molar ratios and diluted with water and ethanol to a final lipid concentration of e.g., between about 5.5 mM and about 25 mM.

### Example 28B: In Vivo Formulation Studies

In order to monitor how effectively various nanoparticle compositions deliver circRNA to targeted cells, different nanoparticle compositions including circRNA are prepared and administered to rodent populations. Mice are intravenously, intramuscularly, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a circRNA in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. Time courses of protein expression can also be evaluated. Samples collected from the rodents for evaluation may include blood and tissue (for example, muscle tissue from the site of an intramuscular injection and internal tissue); sample collection may involve sacrifice of the animals.

Higher levels of protein expression induced by administration of a composition including a circRNA will be indicative of higher circRNA translation and/or nanoparticle composition circRNA delivery efficiencies. As the non-RNA components are not thought to affect translational machineries themselves, a higher level of protein expression is likely indicative of a higher efficiency of delivery of the circRNA by a given nanoparticle composition relative to other nanoparticle compositions or the absence thereof.

### Example 29

### T Cell Targeting

To target transfer vehicles to T cells, T cell antigen binders, e.g., anti-CD8 antibodies, are coupled to the surface of the transfer vehicle. Anti-T cell antigen antibodies are mildly reduced with an excess of DTT in the presence of EDTA in PBS to expose free hinge region thiols. To remove DTT, antibodies are passed through a desalting column. The heterobifunctional cross-linker SM(PEG)24 is used to anchor antibodies to the surface of circRNA-loaded transfer vehicles (Amine groups are present in the head groups of PEG lipids, free thiol groups on antibodies were created by DTT, SM(PEG)24 cross-links between amines and thiol groups). Transfer vehicles are first incubated with an excess of SM(PEG)24 and centrifuged to remove unreacted cross-linker. Activated transfer vehicles are then incubated with an excess of reduced anti-T cell antigen antibody. Unbound antibody is removed using a centrifugal filtration device.

### Example 30

circRNA Formulation Using Preformed Vesicles

Cationic lipid containing transfer vehicles are made using the preformed vesicle method. Cationic lipid, DSPC, cholesterol and PEG-lipid are solubilized in ethanol at a molar ratio of 40/10/40/10, respectively. The lipid mixture is added to an aqueous buffer (50 mM citrate, pH 4) with mixing to a final ethanol and lipid concentration of 30% (vol/vol) and 6.1 mg/mL respectively and allowed to equilibrate at room temperature for 2 min before extrusion. The hydrated lipids are extruded through two stacked 80 nm pore-sized filters (Nuclepore) at 22° C. using a Lipex Extruder (Northern Lipids, Vancouver, BC) until a vesicle diameter of 70-90 nm, as determined by Nicomp analysis, is obtained. For cationic lipid mixtures which do not form small vesicles, hydrating the lipid mixture with a lower pH buffer (50 mM citrate, pH 3) to protonate the phosphate group on the DSPC headgroup helps form stable 70-90 nm vesicles.

The FVII circRNA (solubilized in a 50 mM citrate, pH 4 aqueous solution containing 30% ethanol) is added to the vesicles, pre-equilibrated to 35° C., at a rate of ˜5 mL/min with mixing. After a final target circRNA/lipid ratio of 0.06 (wt wt) is achieved, the mixture is incubated for a further 30 min at 35° C. to allow vesicle re-organization and encapsulation of the FVII RNA. The ethanol is then removed and the external buffer replaced with PBS (155 mM NaCl, 3 mM Na2HP04, ImM KH2P04, pH 7.5) by either dialysis or tangential flow diafiltration. The final encapsulated circRNA-to-lipid ratio is determined after removal of unencapsulated RNA using size-exclusion spin columns or ion exchange spin columns.

### Example 31

Expression of Engineered Circular RNA with a Half-Life Extension Domain has Improved Pharmacokinetic Parameters than without a Half-Life Extension Domain

The trispecific antigen-binding protein encoded on a circRNA molecule of example 23 is administered to cynomolgus monkeys as a 0.5 mg/kg bolus injection intramuscularly. Another cynomolgus monkey group receives a comparable protein encoded on a circRNA molecule in size with binding domains to CD3 and GPC-3, but lacking a half-life extension domain. A third and fourth group receive a protein encoded on a circRNA molecule with CD3 and half-life extension domain binding domains and a protein with GPC-3 and half-life extension domains, respectively. Both proteins encoded by circRNA are comparable in size to the trispecific antigen-binding protein. Each test group consists of 5 monkeys. Serum samples are taken at indicated time points, serially diluted, and the concentration of the proteins is determined using a binding ELISA to CD3 and/or GPC-3.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=Ae^{˜at}+Be^{˜pt}, where c(t) is the plasma concentration at time t, A and B are intercepts on the Y-axis, and a and β are the apparent first-order rate constants for the distribution and elimination phases, respectively. The a-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(a−k21)/(a−p), B=D/V(p−k21)/(a−p), and a and β (for α>β) are roots of the quadratic equation: r²+(k12+k21+k10)r+k21k10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KaleidaGraph™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WinNonlin® Professional V. 3.1 WinNonlin™ Copyright 1998-1999. Pharsight Corporation. Mountain View, Calif). Pharmacokinetic parameters are computed as described in Ritschel WA and Kearns G L, 1999, EST: Handbook Of Basic Pharmacokinetics Including Clinical Applications, 5th edition, American Pharmaceutical Assoc., Washington, DC.

It is expected that the trispecific antigen-binding protein encoded on a circRNA molecule of Example 23 has improved pharmacokinetic parameters such as an increase in elimination half-time as compared to proteins lacking a half-life extension domain.

### Example 32

### In Vivo Analysis

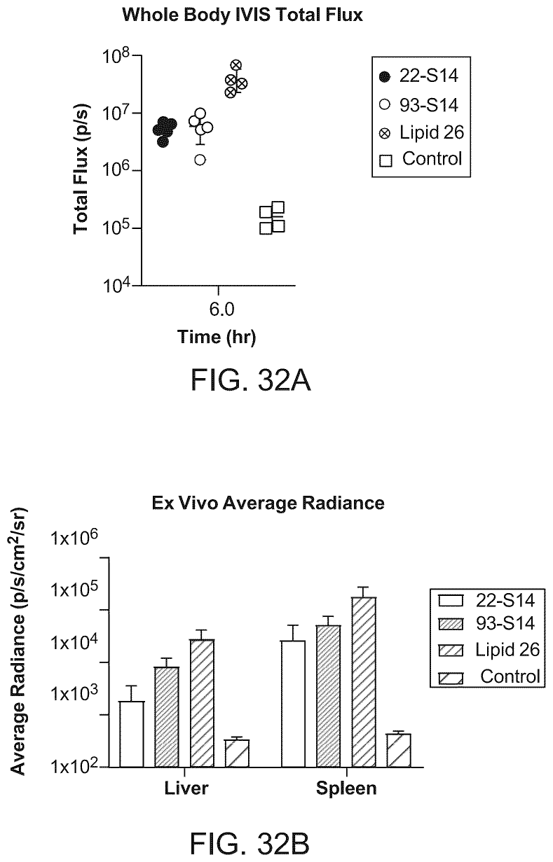

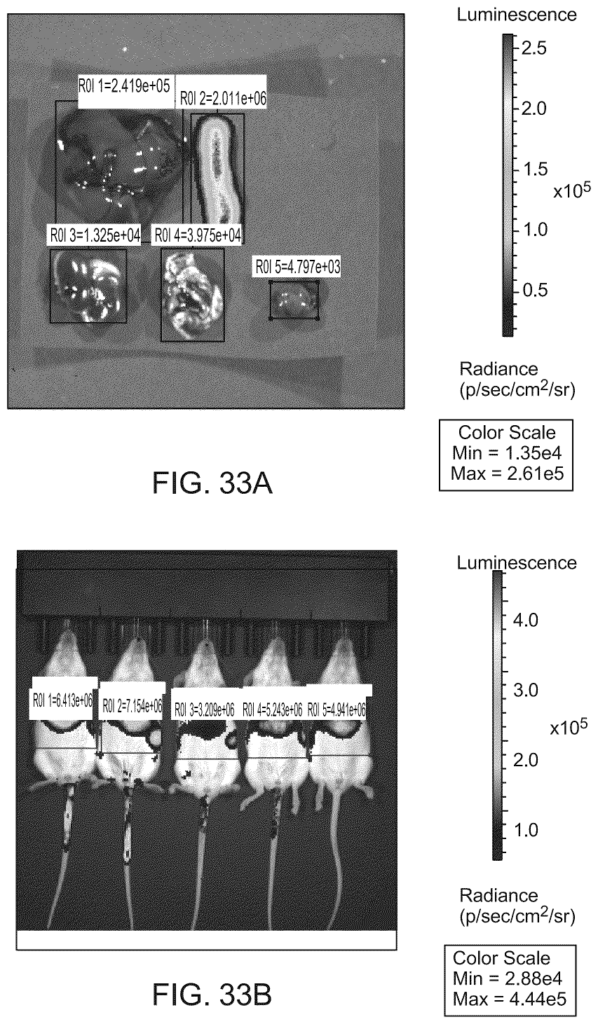

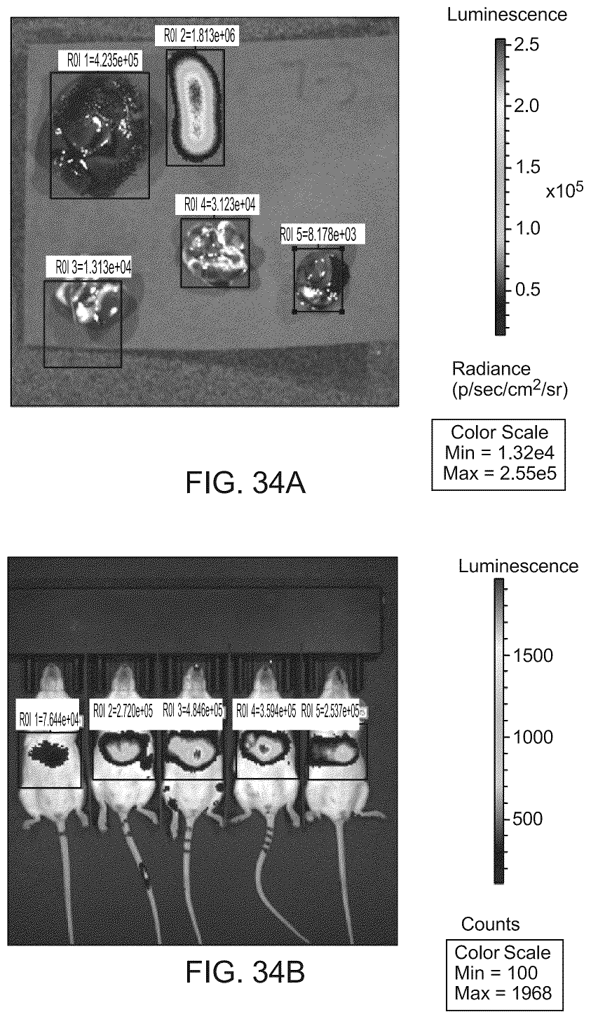

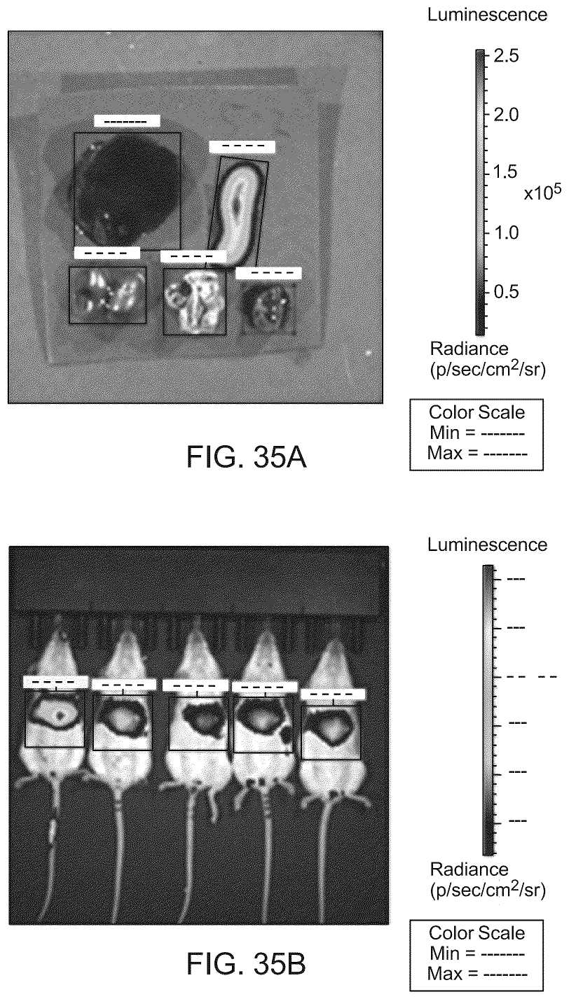

Female CD-1 or female c57BL/6J mice ranging from 22-25 g were dosed at 0.5 mg/kg RNA intravenously. Six hours after injection, mice were injected intraperitoneally with 200 μL of D-luciferin at 15 mg/mL concentration. 5 minutes after injection, mice were anesthetized using isoflurane, and placed inside the IVIS Spectrum In Vivo Imaging System (Perkin Elmer) with dorsal side up. Whole body total IVIS flux of Lipids 22-S14, 93-S14, Lipid 10a-26 is presented in FIG. 32A. Post 10 minutes injection, mice were scanned for luminescence. Mice were euthanized and organs were extracted within 25 minutes of luciferin injection to scan for luminescence in liver, spleen, kidneys, lungs, and heart. Images (FIGS. 33A-B, 34A-B, 35A-B) were analyzed using Living Images (Perkin Elmer) software. Regions of interest were drawn to obtain flux and average radiance and analyzed for biodistribution of protein expression (FIG. 32A-B).

FIG. 32A illustrates the increased whole-body total flux observed from luciferase circRNA with Lipid 10a-26 LNPs compared to LNPs made with lipids 22-S14 and 93-S14. FIG. 32B shows the ex vivo IVIS analysis of tissues further highlighting the increased overall expression with Lipid 10a-26 while maintaining the desired spleen to liver ratios observed with lipids 22-S14 and 93-S14 despite the significant structural changes designed to improve expression. These data highlight the improvements afforded by Lipid 10a-26 compared to previously reported lipids.

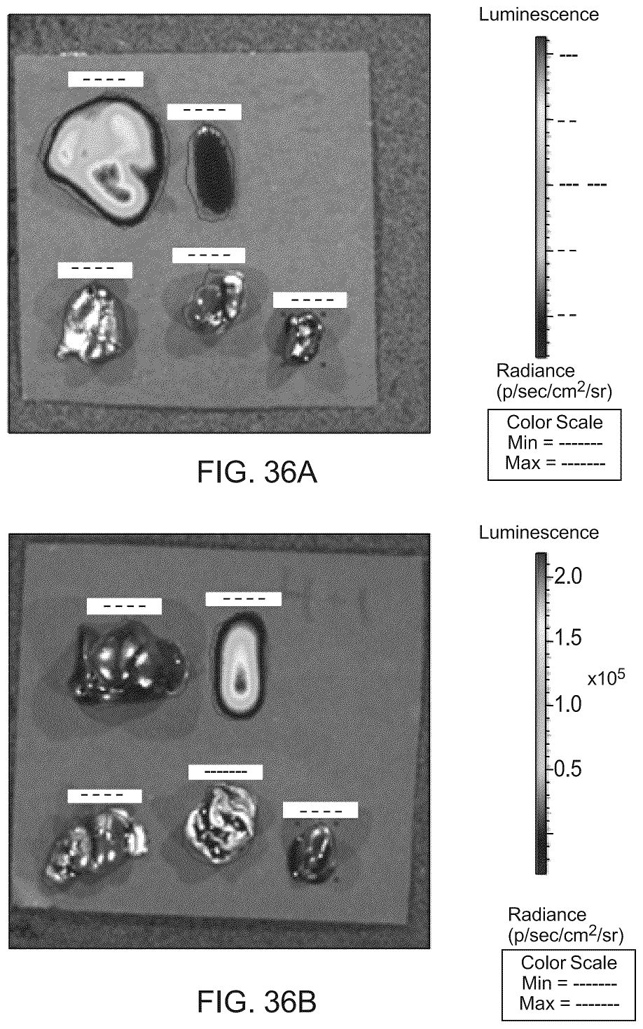

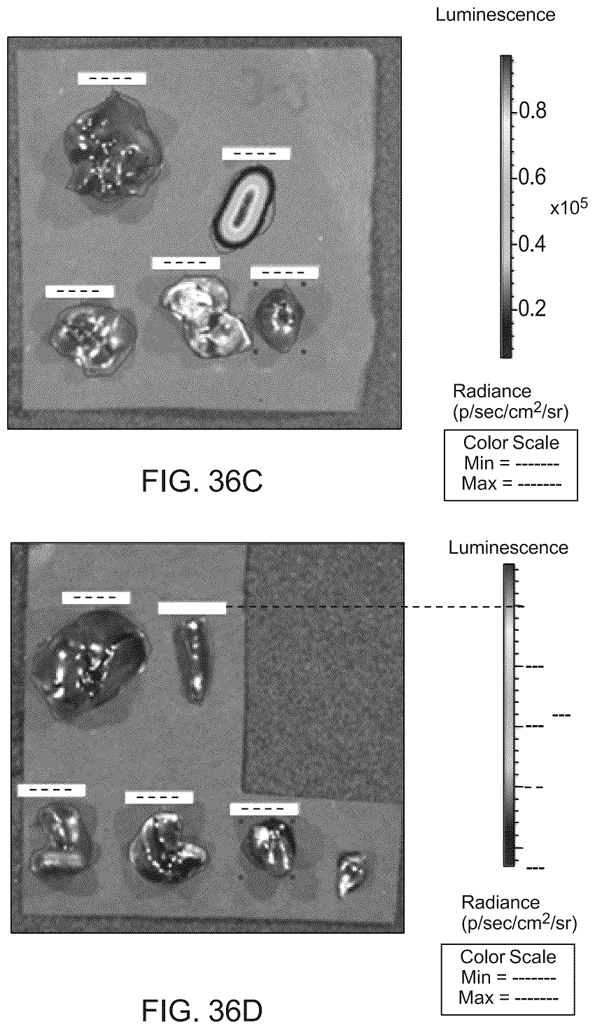

Similar analysis as described above was also performed with circRNA encapsulated in LNPs formed with Lipid 10b-15 or Lipid 10a-53 or 10a-54. FIGS. 36A-C show the ex vivo IVIS analysis of tissues, respectively highlighting the overall expression with Lipid 10b-15, 10a-53, and 10a-54 while maintaining the desired spleen to liver ratios despite the significant structural changes designed to improve expression. FIG. 36D shows the results for PBS control. These data demonstrates the improvements afforded by Lipids 10b-15, 10a-53, and 10a-54 compared to previously reported lipids such as 93-S14 and 22-S14.

### Example 33

### Delivery of Luciferase

Human peripheral blood mononuclear cells (PBMCs) (Stemcell Technologies) were transfected with lipid nanoparticles (LNP) encapsulating firefly luciferase (f. luc) circular RNA and examined for luciferase expression. PBMCs from two different donors were incubated in vitro with five different LNP compositions, containing circular RNA encoding for firefly luciferase (200 ng), at 37° C. in RPMI, 2% human serum, IL-2 (10 ng/mL), and 50 uM BME. PBMCs incubated without LNP were used as a negative control. After 24 hours, the cells were lysed and analyzed for firefly luciferase expression based on bioluminescence (Promega BrightGlo).

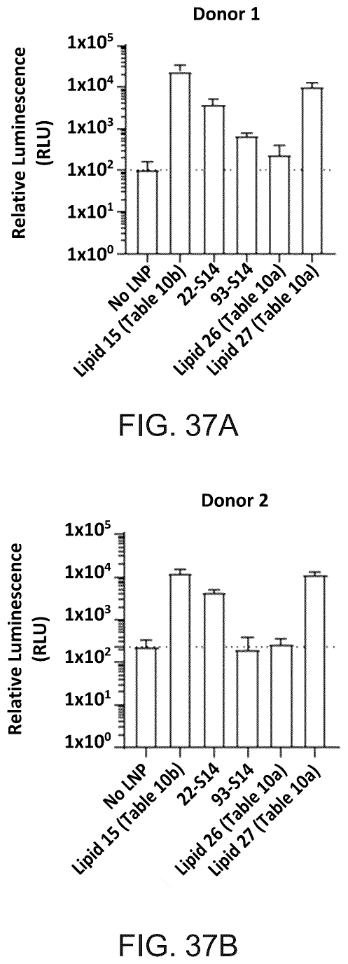

Representative data are presented in FIGS. 37A and 37B, showing that that the tested LNPs are capable of delivering circular RNA into primary human immune cells resulting in protein expression.

### Example 34

### In Vitro Delivery of Green Fluorescent Protein (GFP) or Chimeric Antigen Receptor (CAR)

Human PBMCs (Stemcell Technologies) were transfected with LNP encapsulating GFP and examined by flow cytometry. PBMCs from five different donors (PBMC A-E) were incubated in vitro with one LNP composition, containing circular RNA encoding either GFP or CD19-CAR (200 ng), at 37° C. in RPMI, 2% human serum, IL-2 (10 ng/mL), and 50 uM BME. PBMCs incubated without LNP were used as a negative control. After 24, 48, or 72 hours post-LNP incubation, cells were analyzed for CD3, CD19, CD56, CD14, CD11b, CD45, fixable live dead, and payload (GFP or CD19-CAR).

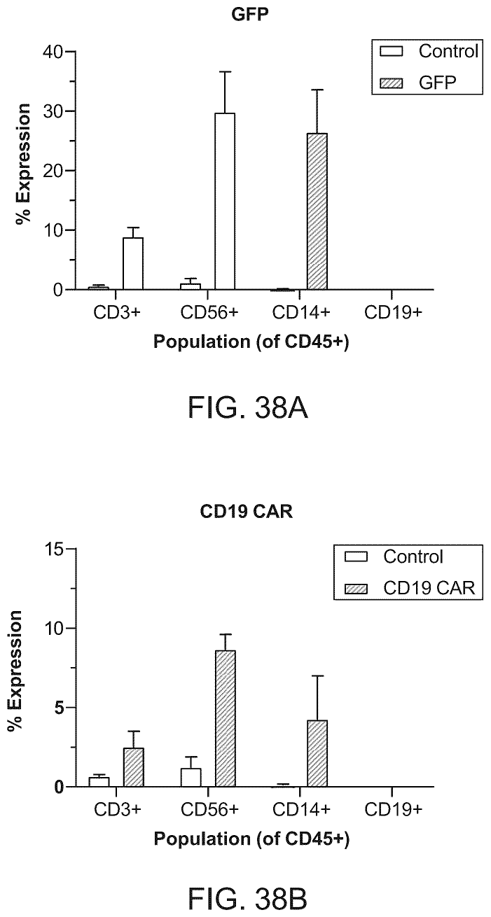

Representative data are presented in FIGS. 38A and 38B, showing that the tested LNP is capable of delivering circular RNA into primary human immune cells resulting in protein expression.

### Example 35

### Multiple IRES Variants can Mediate Expression of Murine CD19 CAR In Vitro

Multiple circular RNA constructs, encoding anti-murine CD19 CAR, contains unique IRES sequences and were lipotransfected into 1C1C7 cell lines. Prior to lipotransfection, 1C1C7 cells are expanded for several days in complete RPMI Once the cells expanded to appropriate numbers, 1C1C7 cells were lipotransfected (Invitrogen RNAiMAX) with four different circular RNA constructs. After 24 hours, 1C1C7 cells were incubated with His-tagged recombinant murine CD19 (Sino Biological) protein, then stained with a secondary anti-His antibody. Afterwards, the cells were analyzed via flow cytometry.

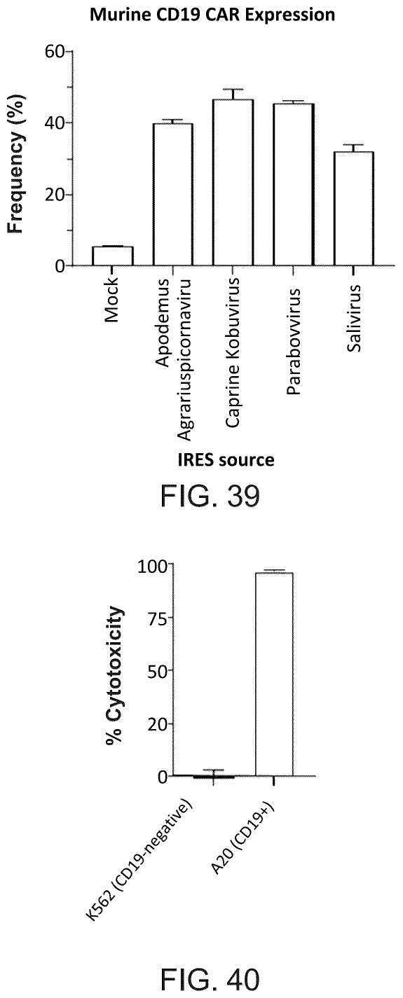

Representative data are presented in FIGS. 39, showing that IRES sourced from the indicated virus (apodemus agrarius picornavirus, caprine kobuvirus, parabovirus, and salivirus) are capable of driving expression of an anti-mouse CD19 CAR in murine T cells.

### Example 36

### Murine CD19 CAR Mediates Tumor Cell Killing In Vitro

Circular RNA encoding anti-mouse CD19 CAR were electroporated into murine T cells to evaluate CAR-mediated cytotoxicity. For electroporation, T cells were electroporated with circular RNA encoding anti-mouse CD19 CAR using ThermoFisher's Neon Transfection System then rested overnight. For the cytotoxicity assay, electroporated T cells were co-cultured with Fluc+ target and non-target cells at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega Brightglo Luciferase System) to detect lysis of Fluc+ target and non-target cells. Values shown are calculated relative to the untransfected mock signal.

Representative data are presented in FIG. 40, showing that an anti-mouse CD19 CAR expressed from circular RNA is functional in murine T cells in vitro.

### Example 37

Functional Depletion of B Cells with a Lipid Encapsulated Circular RNA Encoding Murine CD19 CAR

C57BL/6J mice were injected with LNP formed with Lipid 10b-15, encapsulating circular RNA encoding anti-murine CD19 CAR. As a control, Lipid 10b-15 encapsulating circular RNA encoding firefly luciferase (f.Luc) were injected in different group of mice. Female C57BL.6J, ranging from 20-25 g, were injected intravenously with 5 doses of 0.5 mg/kg of LNP, every other day. Between injections, blood draws were analyzed via flow cytometry for fixable live/dead, CD45, TCRvb, B220, CD1 ib, and anti-murine CAR. Two days after the last injection, spleens were harvested and processed for flow cytometry analysis. Splenocytes were stained with fixable live/dead, CD45, TCRvb, B220, CD1 ib, NK1.1, F4/80, CD11c, and anti-murine CAR. Data from mice injected with anti-murine CD19 CAR LNP were normalized to mice that receivedf.Luc LNP.

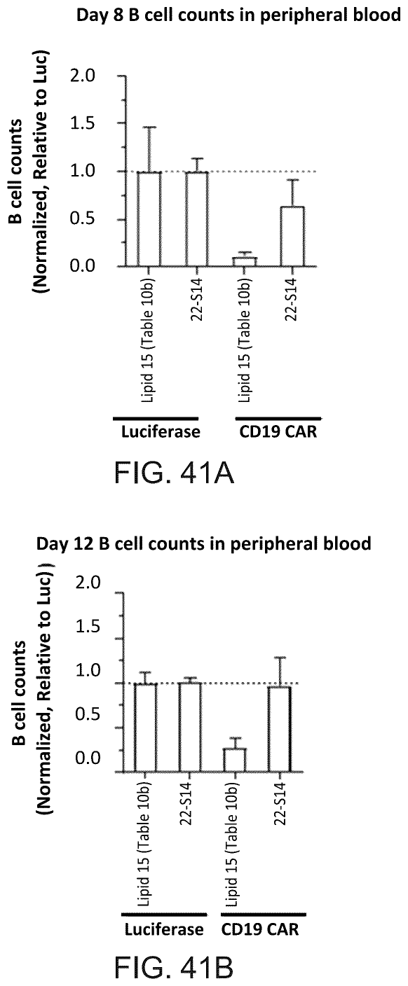

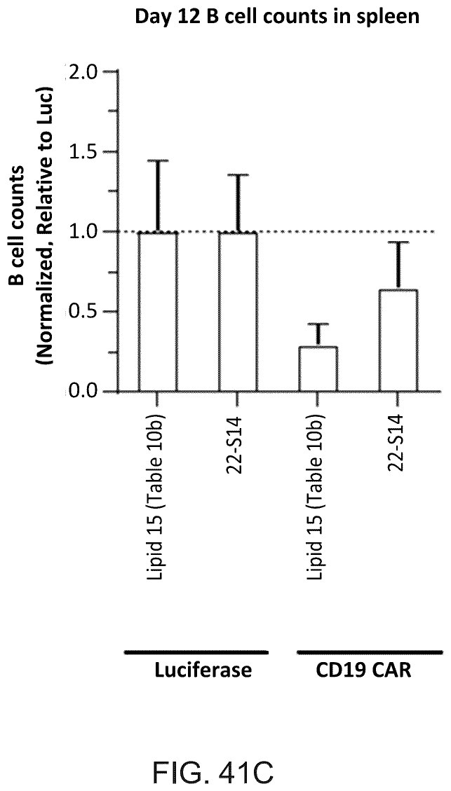

Representative data are presented in FIGS. 41A, 41B, and 41C, showing that an anti-mouse CD19 CAR expressed from circular circRNA delivered in vivo with LNPs is functional in murine T cells in vivo.

### Example 38

Cd19 CAR Expressed from Circular RNA has Higher Yield and Greate Cytotoxic Effect Compared to that Expressed from mRNA

Circular RNA encoding an anti-CD19 chimeric antigen receptor, which includes, from N-terminus to C-terminus, a FMC63-derived scFv, a CD8 transmembrane domain, a 4-1BB costimulatory domain, and a CD3ζ intracellular domain, were electroporated into human peripheral T cells to evaluate surface expression and CAR-mediated cytotoxicity. For comparison, circular RNA-electroporated T cells were compared to mRNA-electroporated T cells in this experiment. For electroporation, CD3+ T cells were isolated from human PBMCs using commercially available T cell isolation kits (Miltenyi Biotec) from donor human PBMCs. After isolation, T cells were stimulated with anti-CD3/anti-CD28 (Stemcell Technologies) and expanded over 5 days at 37° C. in complete RPMI containing 10% FBS, IL-2 (10 ng/mL), and 5 μM BME. Five days post stimulation, T cells were electroporated with circular RNA encoding anti-human CD19 CAR using ThermoFisher's Neon Transfection System and then rested overnight. For the cytotoxicity assay, electroporated T cells were co-cultured with Fluc+ target and non-target cells at 1:1 ratio in complete RPMI containing 10% FBS, IL-2 (10 ng/mL), and 5 μM BME and incubated overnight at 37° C. Cytotoxicity was measured using a luciferase assay system 24 hours post-co-culture (Promega Brightglo Luciferase System) to detect lysis of Fluc+ target and non-target cells. Furthermore, an aliquot of electroporated T cells were taken and stained for live dead fixable staining, CD3, CD45, and chimeric antigen receptors (FMC63) at the day of analysis.

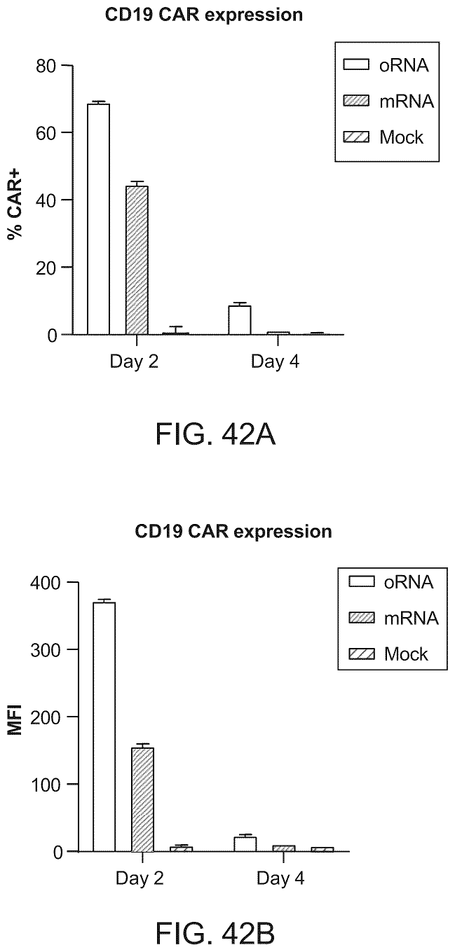

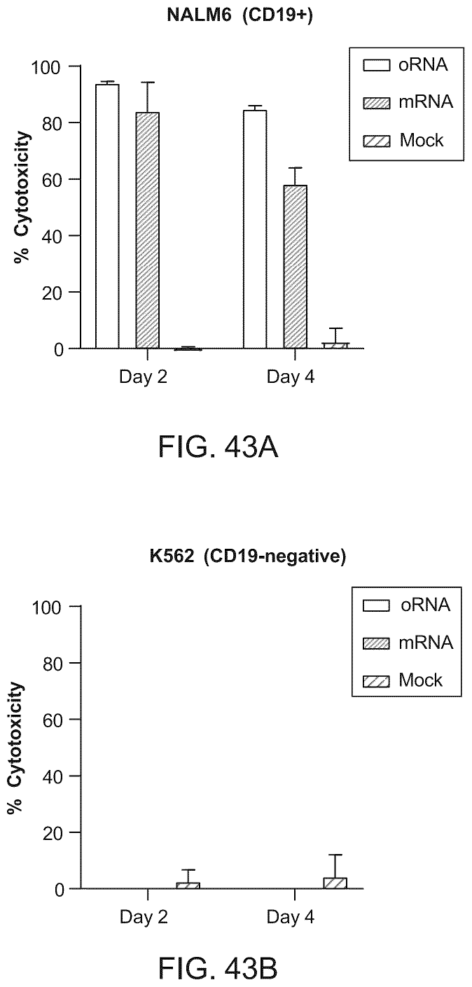

Representative data are presented in FIGS. 42 and 43. FIGS. 42A and 42B show that an anti-human CD19 CAR expressed from circular RNA is expressed at higher levels and longer than an anti-human CD19 CAR expressed from linear mRNA. FIGS. 43A and 43B show that an anti-human CD19 CAR expressed from circular RNA is exerts a greater cytotoxic effect relative to anti-human CD19 CAR expressed from linear mRNA.

### Example 39

Functional Expression of Two CARs from a Single Circular RNA

Circular RNA encoding chimeric antigen receptors were electroporated into human peripheral T cells to evaluate surface expression and CAR-mediated cytotoxicity. The purpose of this study is to evaluate if circular RNA encoding for two CARs can be stochastically expressed with a 2A (P2A) or an IRES sequence. For electroporation, CD3+ T cells were commercially purchased (Cellero) and stimulated with anti-CD3/anti-CD28 (Stemcell Technologies) and expanded over 5 days at 37° C. in complete RPMI containing 10% FBS, IL-2 (10 ng/mL), and 50 uM BME. Four days post stimulation, T cells were electroporated with circular RNA encoding anti-human CD19 CAR, anti-human CD19 CAR-2A-anti-human BCMA CAR, and anti-human CD19 CAR-IRES-anti-human BCMA CAR using ThermoFisher's Neon Transfection System then rested overnight. For the cytotoxicity assay, electroporated T cells were co-cultured with Fluc+K562 cells expressing human CD19 or BCMA antigens at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega BrightGlo Luciferase System) to detect lysis of Fluc+ target cells.

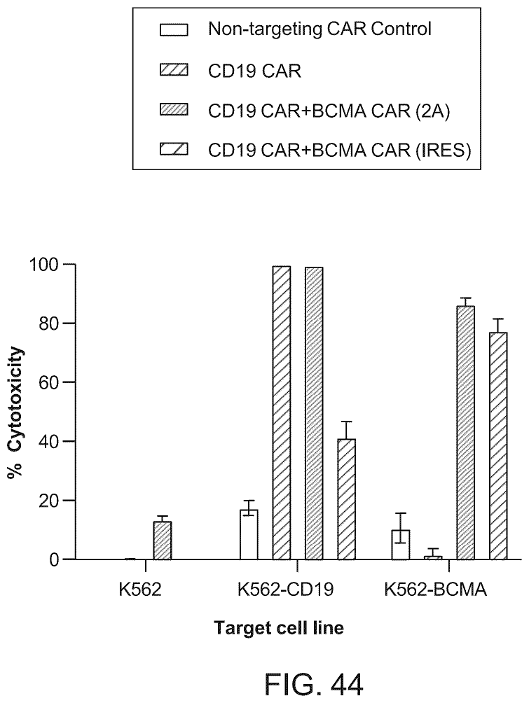

Representative data are presented in FIG. 44, showing that two CARs can be functionally expressed from the same circular RNA construct and exert cytotoxic effector function.

### Example 40

### In Vivo Circular RNA Transfection Using Cre Reporter Mice

Circular RNAs encoding Cre recombinase (Cre) are encapsulated into lipid nanoparticles as previously described. Female, 6-8 week old B6.Cg-Gt(ROSA)26Sortm9(CAG-tdTomato)Hze/J (Ai9) mice were dosed with lipid nanoparticles at 0.5 mg/kg RNA intravenously. Fluorescent tdTomato protein was transcribed and translated in Ai9 mice upon Cre recombination, meaning circular RNAs have been delivered to and translated in tdTomato+ cells. After 48 hr, mice were euthanized and the spleens were harvested, processed into a single cell suspension, and stained with various fluorophore-conjugated antibodies for immunophenotyping via flow cytometry.

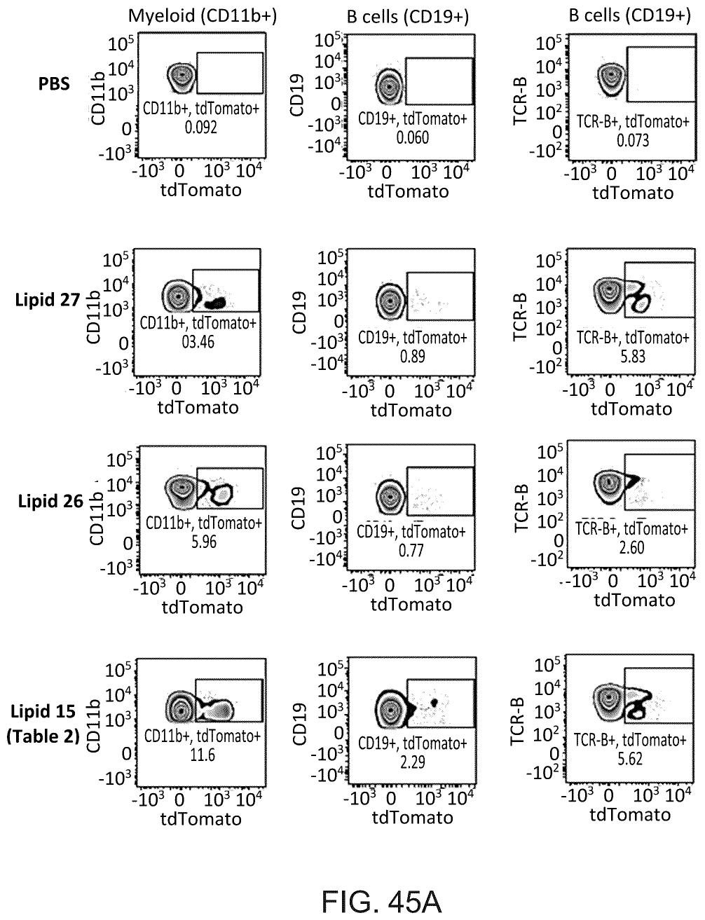

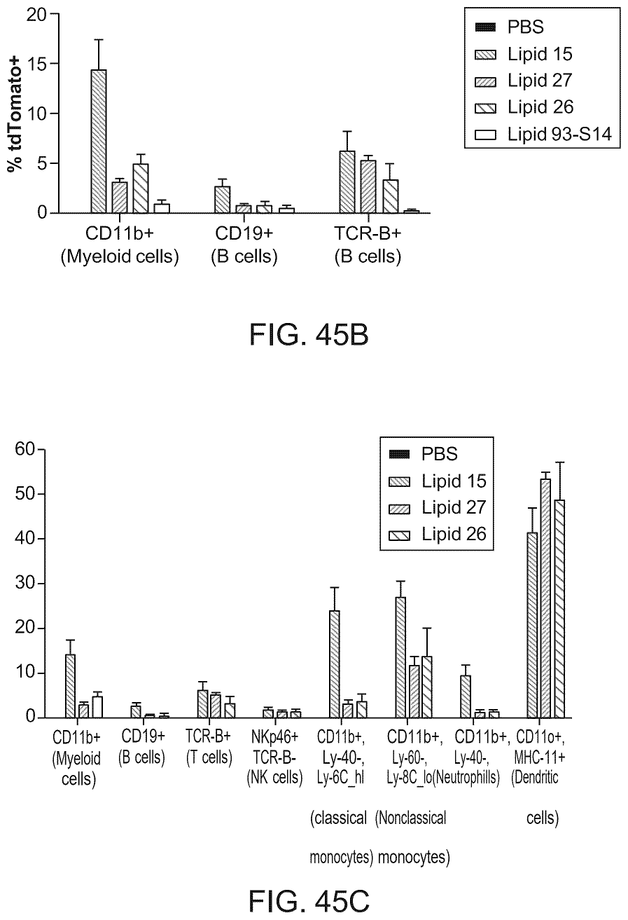

FIG. 45A shows representative FACS plots with frequencies of tdTomato expression in various spleen immune cell (CD45+, live) subsets, including total myeloid (CD11b+), B cells (CD19+), and T cells (TCR-B+) following treatment with LNPs formed with Lipid 10a-27 or 10a-26 or Lipid 10b-15. Ai9 mice injected with PBS represented background tdTomato fluorescence. FIG. 45B quantifies the proportion of myeloid cells, B cells, and T cells expressing tdTomato (mean+std. dev., n=3), which is equivalent to the proportion of each cell population which has been successfully transfected with Cre circular RNA. LNPs made with Lipids 10a-27 and 10a-26 exhibit significantly higher myeloid and T cell transfection compared with Lipid 93-S14, highlighting the improvements conferred by lipid structural modifications.

FIG. 45C illustrates the proportion of additional splenic immune cell populations expressing tdTomato with Lipids 10a-27 and 10a-26 (mean+std. dev., n=3), which also include NK cells (NKp46+, TCR-B−), classical monocytes (CD11b+, Ly-6G-, Ly-6C_hi), nonclassical monocytes (CD11b+, Ly-6G-, Ly-6C_lo), neutrophils (CD11b+, Ly-6G+), and dendritic cells (CD11c+, MHC-II+). These experiments demonstrate that LNPs made with Lipids 10a-27 and 10a-26 and Lipid 10b-15 are effective at delivering circular RNAs to many splenic immune cell subsets in mice and lead to successful protein expression from the circular RNA in those cells.

### Example 41

### Example 41A: Built-In polyA Sequences and Affinity-Purification to Produce Immune-Silent Circular RNA

PolyA sequences (20-30 nt) were inserted into the 5′ and 3′ ends of the RNA construct (precursor RNA with built-in polyA sequences in the introns). See, for example, FIG. 89A. Precursor RNA and introns can alternatively be polyadenylated post-transcriptionally using, e.g., E coli. polyA polymerase or yeast polyA polymerase, which requires the use of an additional enzyme.

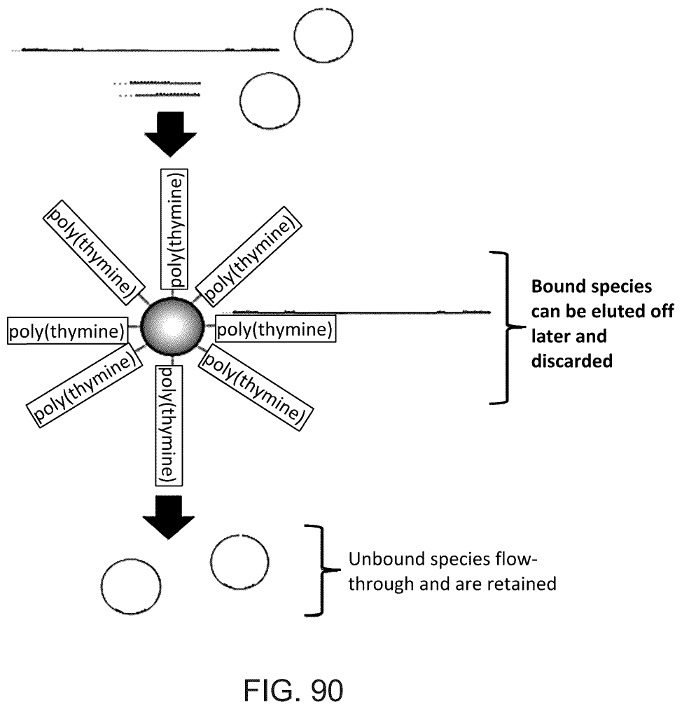

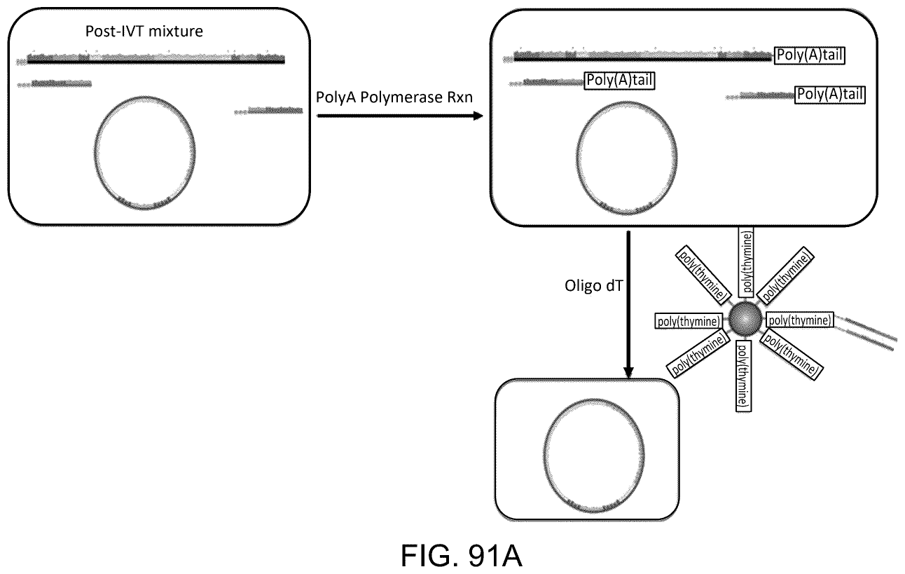

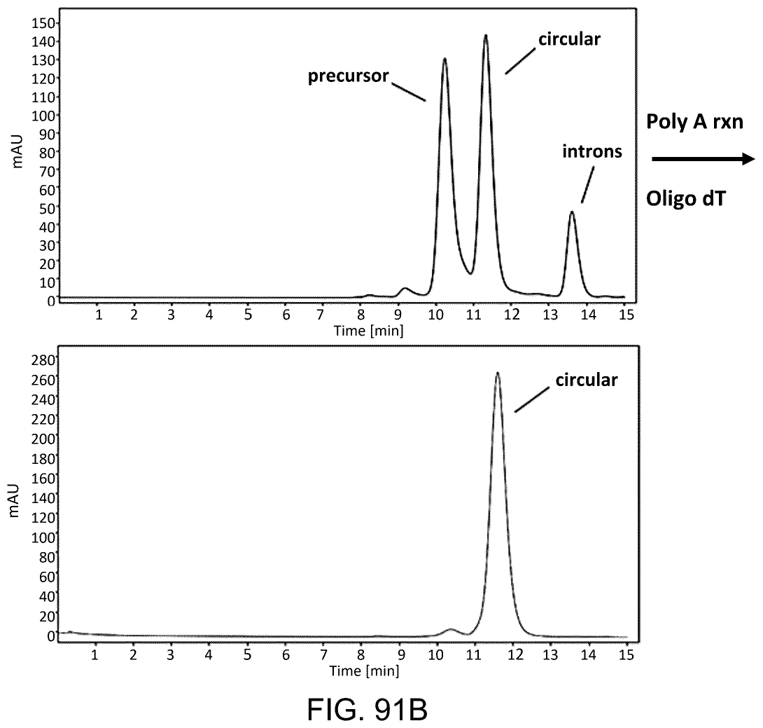

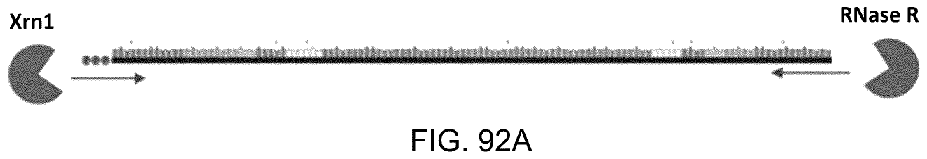

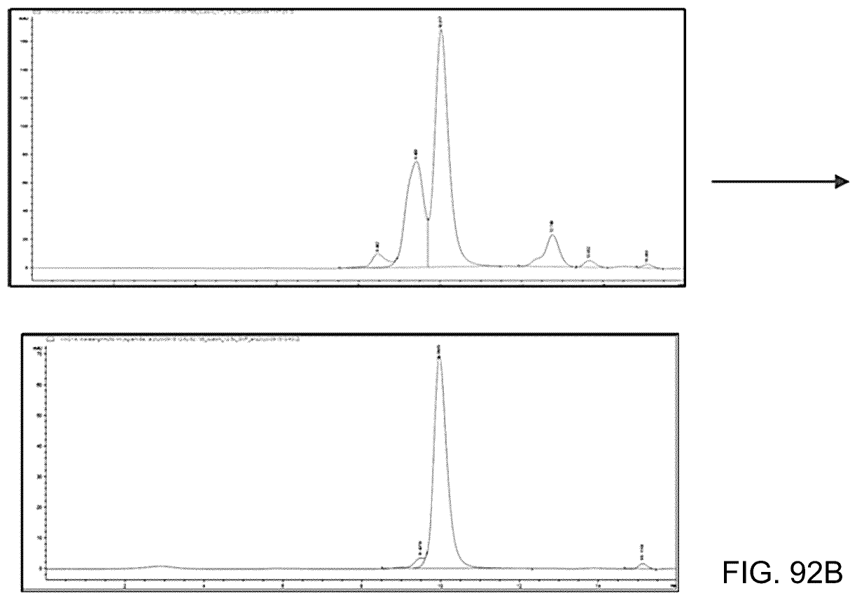

Circular RNA in this example was circularized by in vitro transcription (IVT) and affinity-purified by washing over a commercially available oligo-dT resin to selectively remove polyA-tagged sequences (including free introns and precursor RNA) from the splicing reaction. See, for example, FIG. 90. The IVT was performed with a commercial IVT kit (New England Biolabs) or a customized IVT mix (Orna Therapeutics), containing guanosine monophosphate (GMP) and guanosine triphosphate (GTP) at different ratios (GMP:GTP=8, 12.5, or 13.75). In some embodiments, GMP at a high GMP:GTP ratio may be preferentially included as the first nucleotide, yielding a majority of monophosphate-capped precursor RNAs. As a comparison, the circular RNA product was alternatively purified by the treatment with Xrn1, Rnase R, and Dnase I (enzyme purification). See, for example, FIG. 92.

Immunogenicity of the circular RNAs prepared using the affinity purification or enzyme purification process were then assessed. Briefly, the prepared circular RNAs were transfected into A549 cells. After 24 hours, the cells were lysed and interferon beta-1 induction relative to mock samples was measured by qPCR. 3p-hpRNA, a triphosphorylated RNA, was used as a positive control.

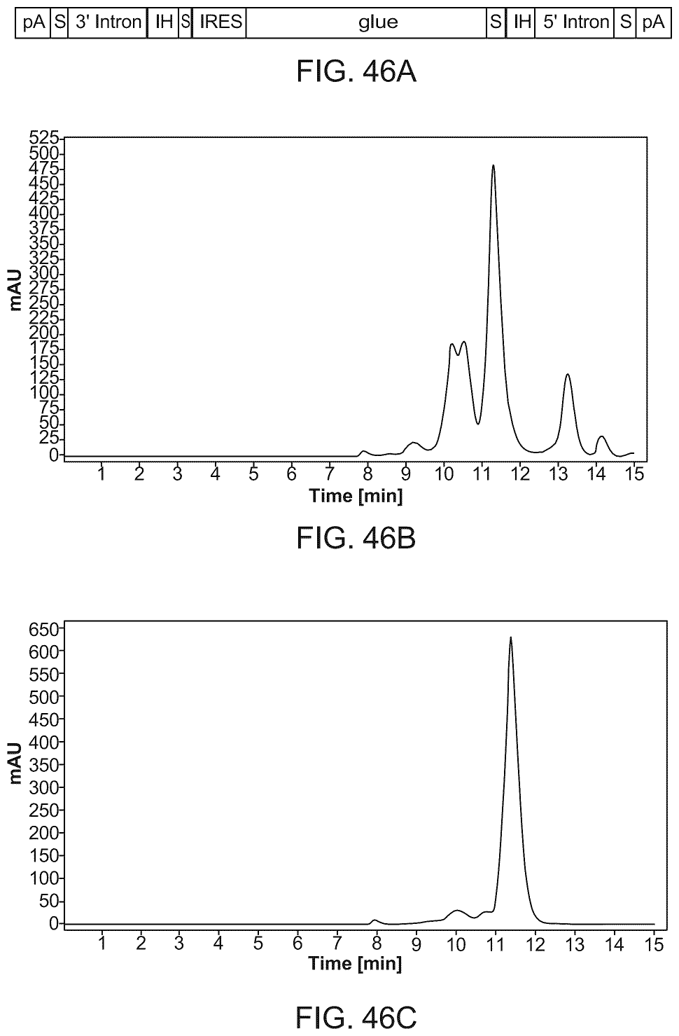

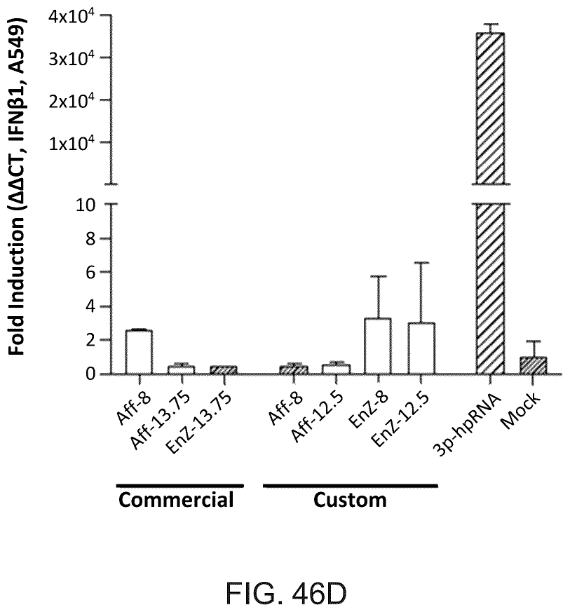

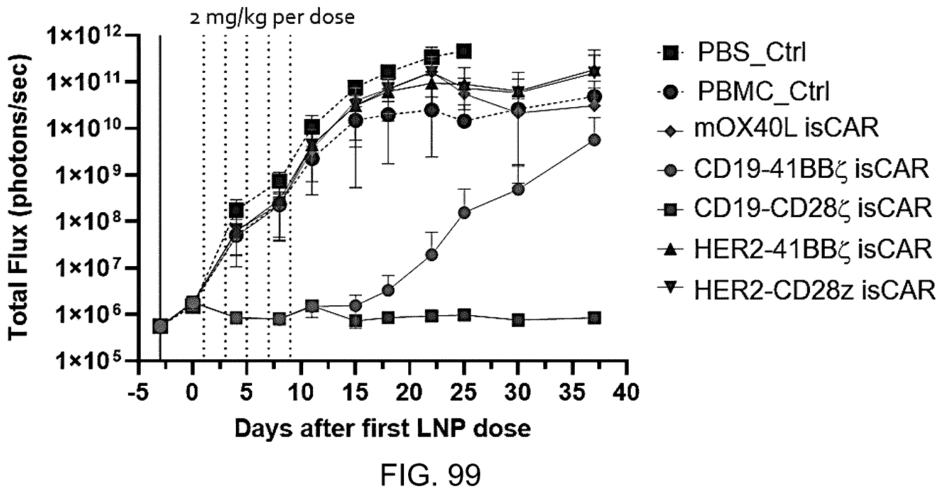

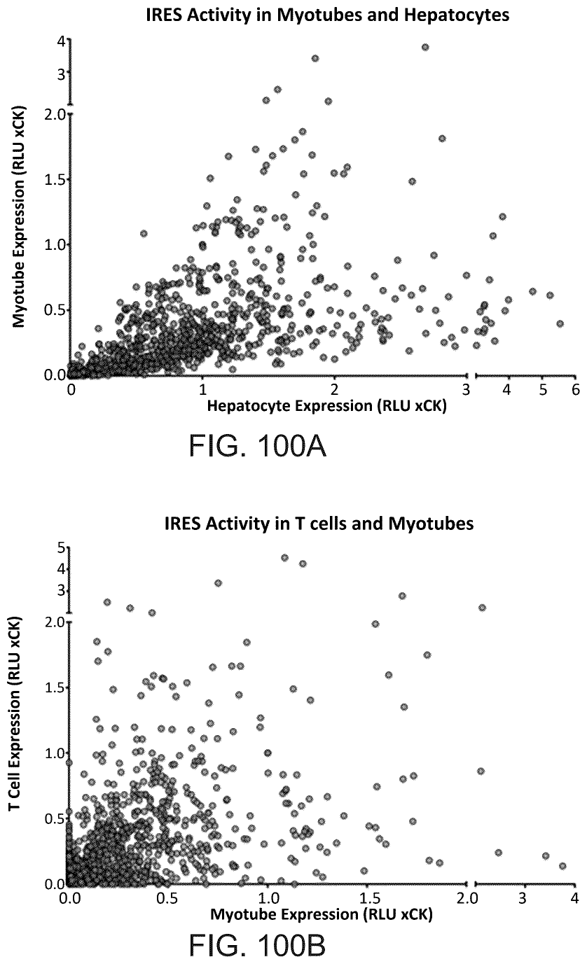

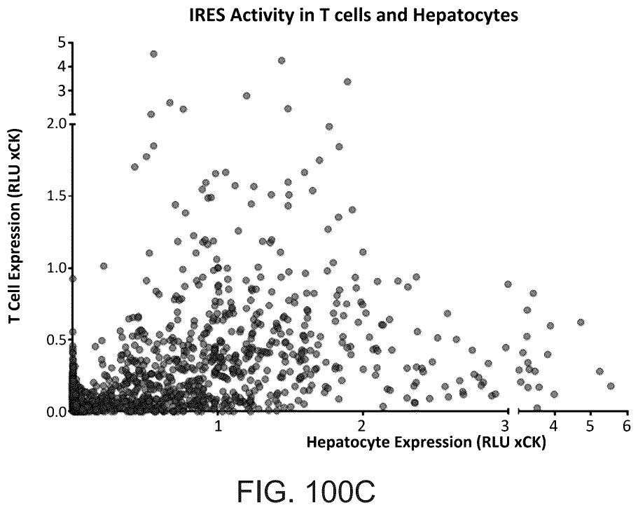

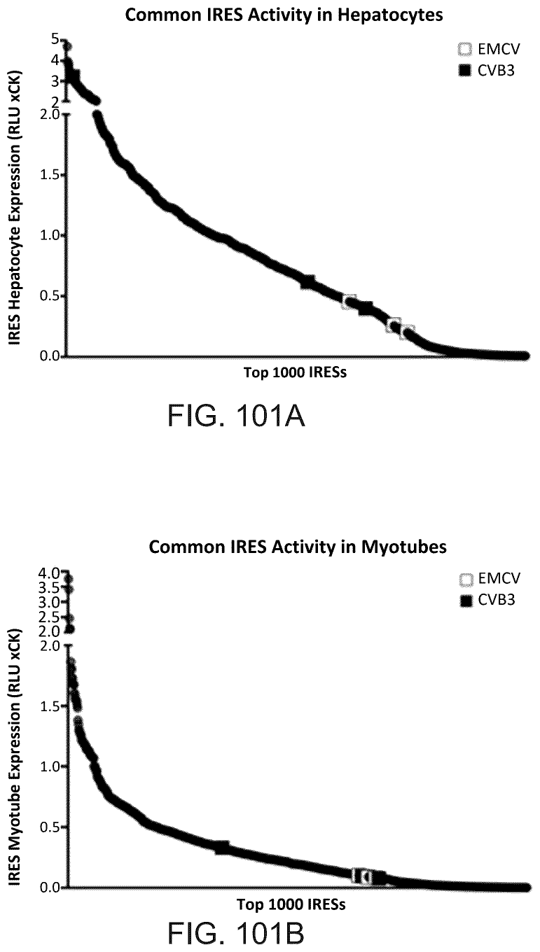

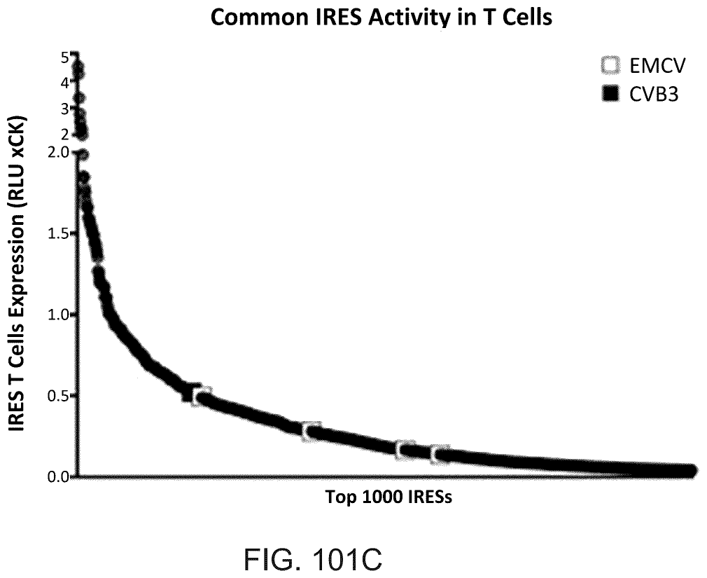

FIGS. 46B and 46C and FIGS. 99A and 99B show that the negative selection affinity purification removes non-circular products from splicing reactions when polyA sequences are included on elements that are removed during splicing and present in unspliced precursor molecules. FIG. 46D shows circular RNAs prepared with tested IVT conditions and purification methods are all immunoquiescent. These results suggest the negative selection affinity purification is equivalent or superior to enzyme purification for circular RNA purification and that customized circular RNA synthesis conditions (IVT conditions) may reduce the reliance on GMP excess to achieve maximal immunoquiescence.

### Example 41B: Dedicated Binding Site and Affinity-Purification for Circular RNA Production

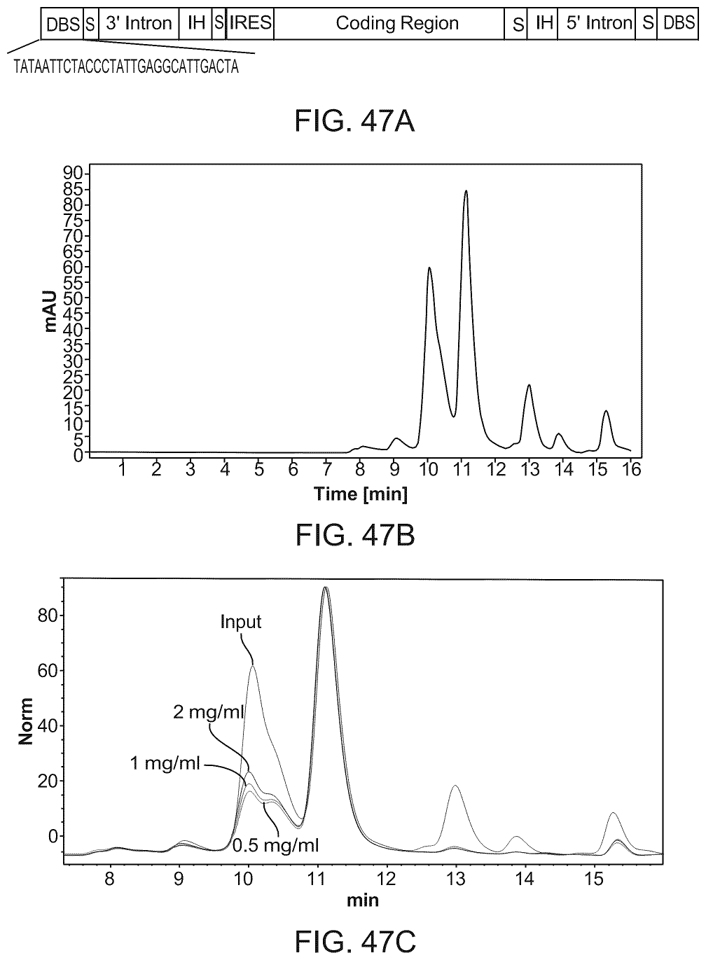

Instead of polyA tags, one can include specifically design sequences (DBS, dedicated binding site). See, for example, FIG. 47A.

Instead of a polyA tag, a dedicated binding site (DBS), such as a specifically designed complementary oligonucleotide that can bind to a resin, may be used to selectively deplete precursor RNA and free introns. In this example, DBS sequences (30 nt) were inserted into the 5′ and 3′ ends of the precursor RNA, such as TATAATTCTACCCTATTGAGGCATTGACTA (SEQ ID NO: 3269), shown in FIG. 47A. RNA was transcribed and the transcribed product was washed over a custom complementary oligonucleotide linked to a resin.

FIGS. 47B and 47C demonstrates that including the designed DBS sequence in elements that are removed during splicing enables the removal of unspliced precursor RNA and free intron components in a splicing reaction, via negative affinity purification.

### Example 41C: Production of a Circular RNA Encoding Dystrophin

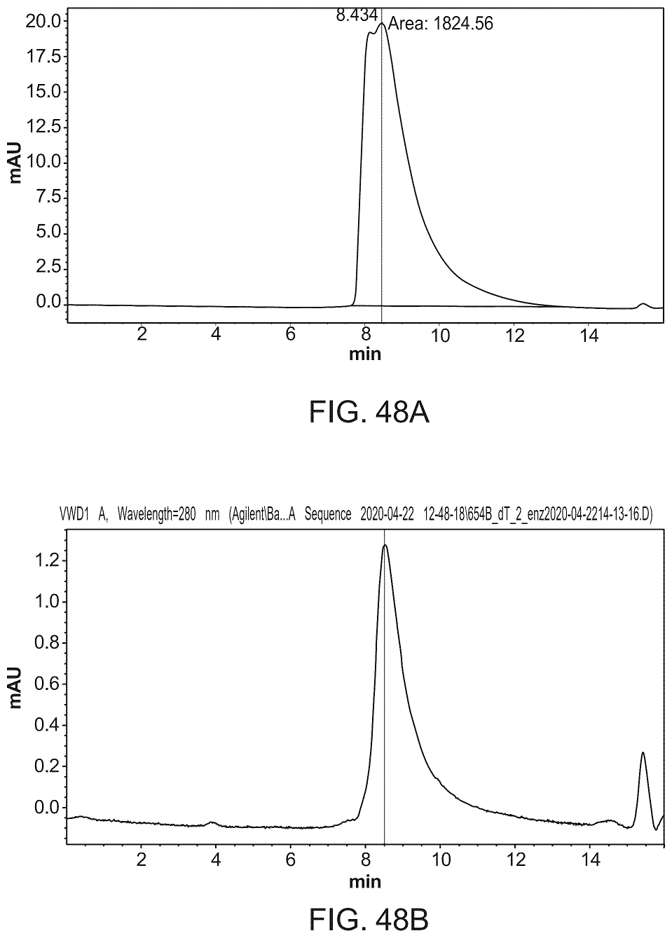

A 12 kb 12,000 nt circular RNA encoding dystrophin was produced by in vitro transcription of RNA precursors followed by enzyme purification using a mixture of Xrn1, DNase 1, and RNase R to degrade remaining linear components. FIG. 48 shows that the circular RNA encoding dystrophin was successfully produced.

### Example 41D: Negative Selection Affinity-Purification of Circular RNA

Affinity sequences are added to precursor RNAs, and circular RNA is produced by in vitro transcription (IVT), as described in Examples 51A and 51B. Oligonucleotides comprising a sequence that is 100% complementary to the affinity sequences are conjugated to a resin, or a commercially available resin conjugated to oligonucleotides complementary to the affinity sequences is obtained. The IVT reaction mixture may be directly added to the resin. Alternatively, the IVT reaction solution is buffer exchanged into binding buffer, such as a Tris buffer, comprising greater than 1 mM one or more monovalent salts, such as NaCl or KCl, or the one or more monovalent salts are simply added to the IVT reaction mixture before the IVT reaction mixture is added to the resin. Circular RNA that does not comprise an affinity sequence is then affinity-purified by washing the solution comprising linear RNA and circular RNA over the resin. The solution may be heated during this process in order to denature aberrantly hybridized sequences. The eluent from the beads is collected and buffer exchanged into water or storage buffer comprising 1 mM sodium citrate, pH 6.5, using a filtration threshold of 10-300 kDa.

### Example 42

### 5′ Spacer Between 3′ Intron Segment and the IRES Improves Circular RNA Expression

Expression level of purified circRNAs with different 5′ spacers between the 3′ intron segment and the IRES in Jurkat cells were compared. Briefly, luminescence from secreted Gaussia luciferase in supernatant was measured 24 hours after electroporation of 60,000 cells with 250 ng of each RNA.

Additionally, stability of purified circRNAs with different 5′ spacers between the 3′ intron segment and the IRES in Jurkat cells were compared. Briefly, luminescence from secreted Gaussia luciferase in supernatant was measured over 2 days after electroporation of 60,000 cells with 250 ng of each RNA and normalized to day 1 expression.

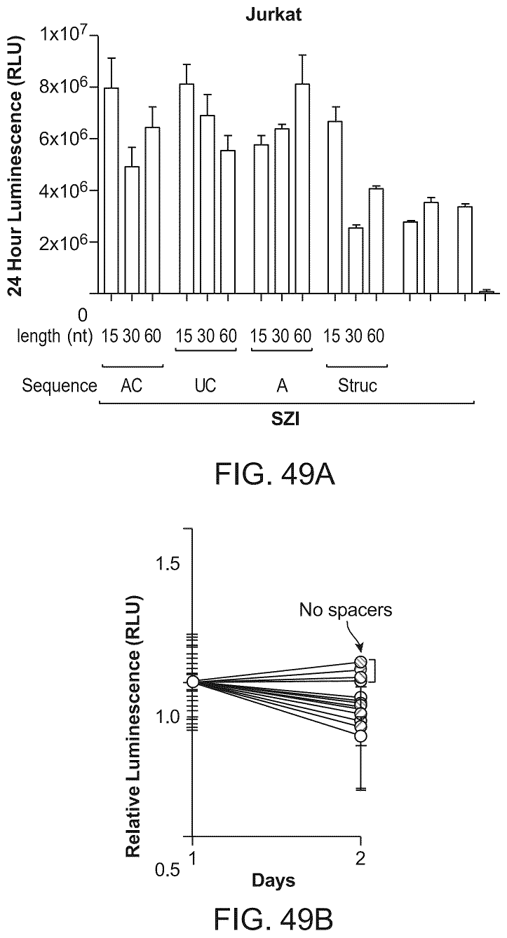

The results are shown in FIGS. 49A and 49B, indicating that adding a spacer can enhance IRES function and the importance of sequence identity and length of the added spacer. A potential explanation is that the spacer is added right before the IRES and likely functions by allowing the IRES to fold in isolation from other structured elements such as the intron segments.

### Example 43

This example describes deletion scanning from 5′ or 3′ end of the caprine kobuvirus TRES. TRES borders are generally poorly characterized and require empirical analysis, and this example can be used for locating the core functional sequences required for driving translation. Briefly, circular RNA constructs were generated with truncated IRES elements operably linked to a Gaussia luciferase coding sequence. The truncated IRES elements had nucleotide sequences of the indicated lengths removed from the 5′ or 3′ end. Luminescence from secreted Gaussia luciferase in supernatant was measured 24 and 48 hours after electroporation of primary human T cells with RNA. Stability of expression was calculated as the ratio of the expression level at the 48-hour time point relative to that at the 24-hour time point.

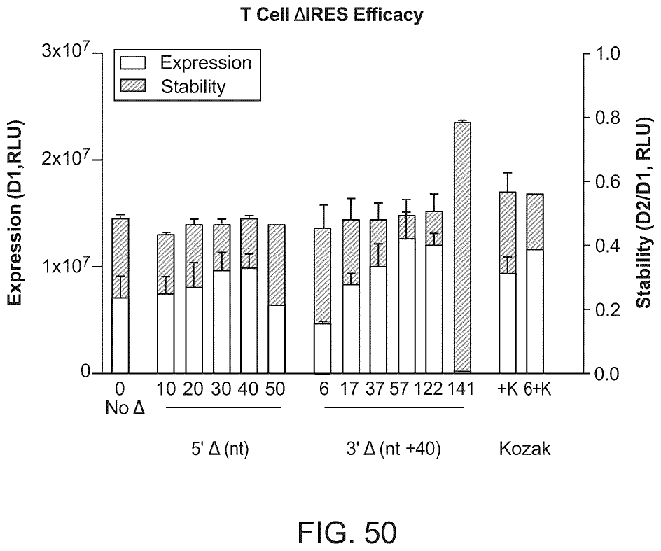

As shown in FIG. 50, deletion of more than 40 nucleotides from the 5′ end of the IRES reduced expression and disrupted IRES function. Stability of expression was relatively unaffected by the truncation of the IRES element but expression level was substantially reduced by deletion of 141 nucleotides from the 3′ end of the IRES, whereas deletion of 57 or 122 nucleotides from the 3′ end had a positive impact on the expression level.

It was also observed that deletion of the 6-nucleotide pre-start sequence reduced the expression level of the luciferase reporter. Replacement of the 6-nucleotide sequence with a classical Kozak sequence (GCCACC) did not have a significant impact but at least maintained expression.

### Example 44

This example describes modifications (e.g., truncations) of selected selected IRES sequences, including Caprine Kobuvirus (CKV) IRES, Parabovirus IRES, Apodemus Picornavirus (AP) IRES, Kobuvirus SZAL6 IRES, Crohivirus B (CrVB) IRES, CVB3 IRES, and SAFV IRES. Briefly, circular RNA constructs were generated with truncated IRES elements operably linked to a Gaussia luciferase coding sequence. HepG2 cells were transfected with the circular RNAs. Luminescence in the supernatant was assessed 24 and 48 hours after transfection. Stability of expression was calculated as the ratio of the expression level at the 48-hour time point relative to that at the 24-hour time point.

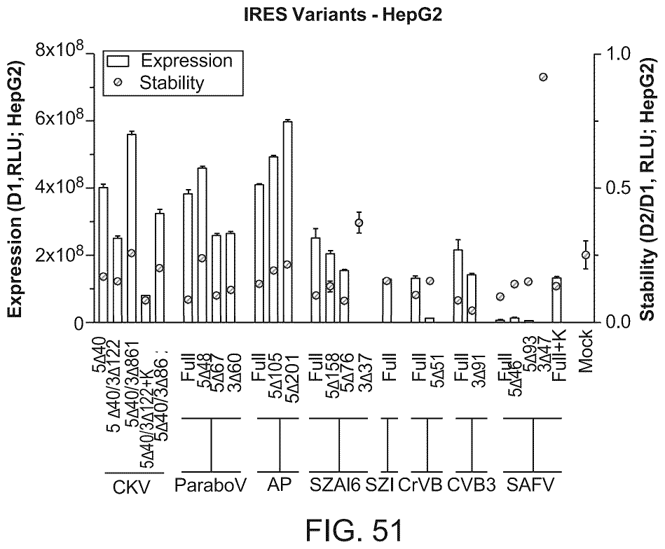

As shown in FIG. 51, truncations had variable effects depending on the identity of the IRES, which may depend on the initiation mechanism and protein factors used for translation, which often differs between IRESs. 5′ and 3′ deletions can be effectively combined, for example, in the context of CKV IRES. Addition of a canonical Kozak sequence in some cases significantly improved expression (as in SAFV, Full vs Full+K) or diminished expression (as in CKV, 5d40/3d122 vs 5d40/3d122+K).

### Example 45

This example describes modifications of CK-739, AP-748, and PV-743 IRES sequences, including mutations and alternative translation initiation sites. Briefly, circular RNA constructs were generated with modified IRES elements operably linked to a Gaussia luciferase coding sequence. Luminescence from secreted Gaussia luciferase in supernatant was measured 24 and 48 hours after transfection of 1C1C7 cells with RNA.

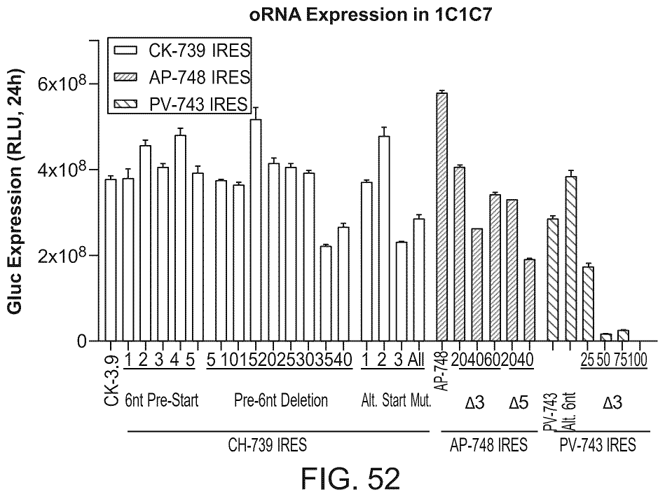

CUG was the most commonly found alternative start site but many others were also characterized. These triplets can be present in the IRES scanning tract prior to the start codon and can affect translation of correct polypeptides. Four alternative start site mutations of the IRES having the sequence of SEQ ID NO: 3282 were created. As shown in FIG. 52, mutations of alternative translation initiation sites in the CK-739 IRES affected translation of correct polypeptides, positively in some instances and negatively in other instances. Mutation of all the alternative translation initiation sites reduced the level of translation.

Alternative Kozak sequences, 6 nucleotides before start codon, can also affect expression levels. The 6-nucleotide sequence upstream of the start codon were gTcacG, aaagtc, gTcacG, gtcatg, gcaaac, and acaacc, respectively, in CK-739 IRES and Sample Nos. 1-5 in the “6 nt Pre-Start” group. As shown in FIG. 52, substitution of certain 6-nucleotide sequences prior to the start codon affected translation.

It was also observed that 5′ and 3′ terminal deletions in AP-748 and PV-743 IRES sequences reduced expression. However, in the CK-739 IRES, which had a long scanning tract, translation was relatively unaffected by deletions in the scanning tract.

### Example 46

This example describes modifications of selected IRES sequences by inserting 5′ and/or 3′ untranslated regions (UTRs) and creating IRES hybrids. Briefly, circular RNA constructs were generated with modified IRES elements operably linked to a Gaussia luciferase coding sequence. Luminescence from secreted Gaussia luciferase in supernatant was measured 24 and 48 hours after transfection of HepG2 cells with RNA.

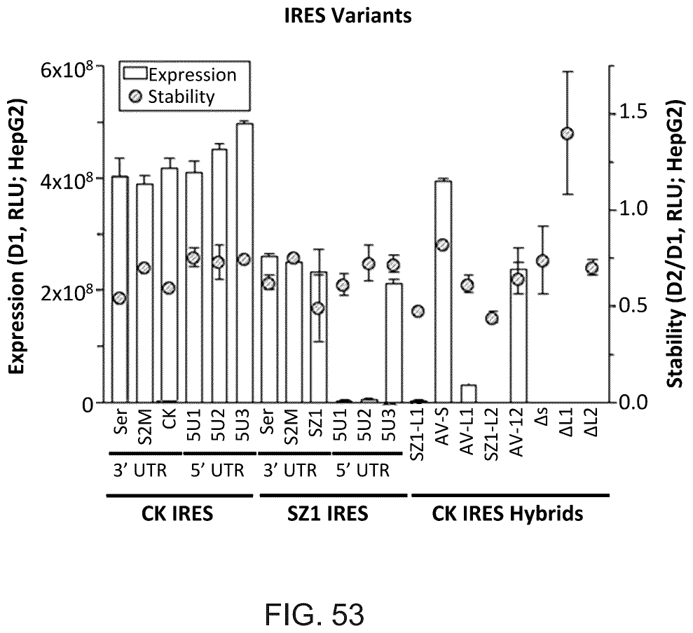

IRES sequences with UTRs inserted may comprise a sequence selected from SEQ ID NOs: 3192-3199. As shown in FIG. 53, insertion of 5′ UTR right after the 3′ end of the IRES and before the start codon slightly increased the translation from Caprine Kobuvirus (CK) IRES but in some instances abrogated translation from Salivirus SZ1 IRES. Insertion of 3′ UTR right after the stop cassette had no impact on both IRES sequences.

Hybrid CK IRES sequences were made with CK IRES as a base, with specific regions of the CK IRES replaced with similar-looking structures from other IRES sequences, for example, SZ1 and AV (Aichivirus). As shown in FIG. 53, certain hybrid synthetic IRES sequences were functional, indicating that hybrid IRES can be constructed using parts from distinct TRES sequences that show similar predicted structures while deleting these structures completely abrogates IRES function.

### Example 47

This example describes modifications of circular RNAs by introducing stop codon or cassette variants. Briefly, circular RNA constructs were generated with IRES elements operably linked to a Gaussia luciferase coding sequence followed by variable stop codon cassettes, which included a stop codon in each frame and two stop codons in the reading frame of the Gaussia luciferase coding sequence. 1C1C7 cells were transfected with the circular RNAs. Luminescence in supernatant was assessed 24 and 48 hours after transfection.

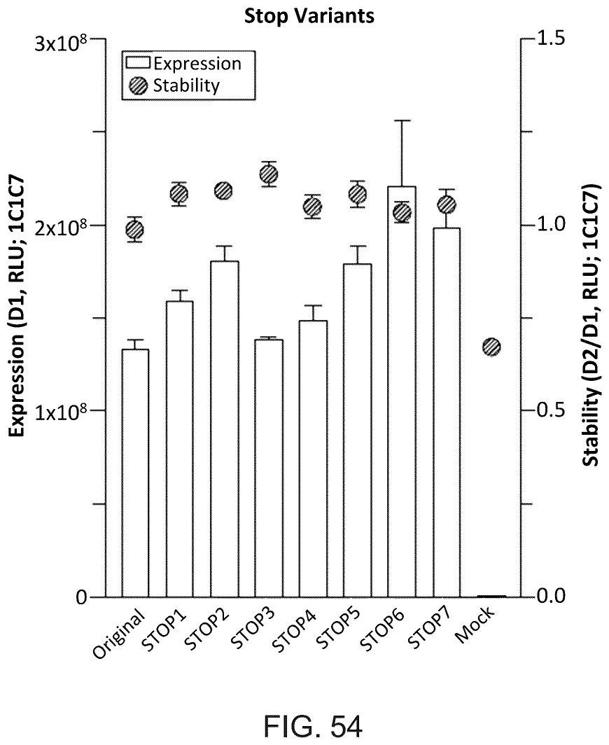

The sequences of the stop codon cassettes comprise a sequence selected from SEQ ID NOs: 3203-3209. As shown in FIG. 54, certain stop codon cassettes improved expression levels, although they had little impact on expression stability. In particular, a stop cassette with two frame 1 (the reading frame of the Gaussia luciferase coding sequence) stop codons, the first being TAA, followed by a frame 2 stop codon and a frame 3 stop codon, is effective for promoting functional translation.

### Example 48

This example describes modifications of circular RNAs by inserting 5′ UTR variants. Briefly, circular RNA constructs were generated with IRES elements with 5′ UTR variants inserted between the 3′ end of the IRES and the start codon, the IRES being operably linked to a Gaussia luciferase coding sequence. 1C1C7 cells were transfected with the circular RNAs. Luminescence in supernatant was assessed 24 and 48 hours after transfection.

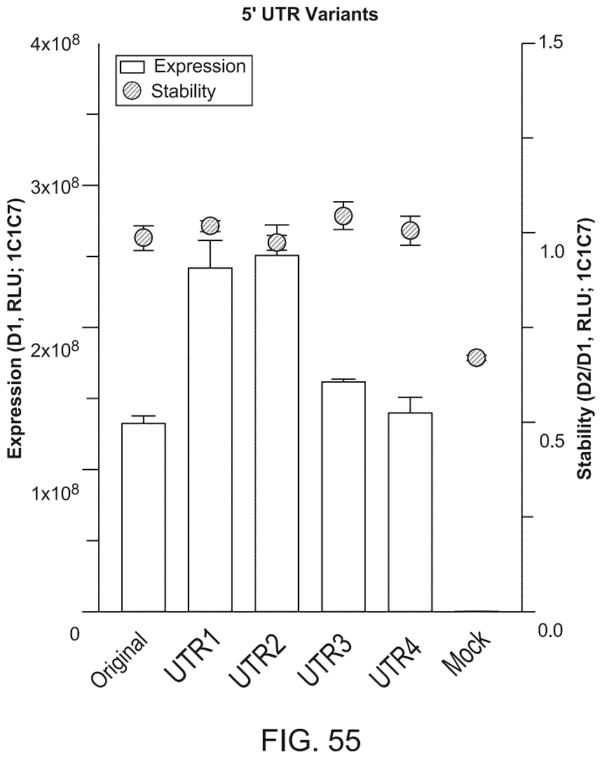

The sequences of the 5′ UTR variants comprise a sequence selected from SEQ ID NOs: 3192-3202. As shown in FIG. 55, a CK TRES with a canonical Kozak sequence (UTR4) was more effective when a 36-nucleotide unstructured/low GC spacer sequence was added (UTR2), suggesting that the GC-rich Kozak sequences may interfere with core TRES folding. Using a higher-GC/structured spacer with a kozak sequence did not show the same benefit (UTR3), possibly due to interference with IRES folding by the spacer itself. Mutating the kozak sequence to gTcacG (UTR1) enhanced translation to the same level as the Kozak+spacer alternative without the need for a spacer.

### Example 49

This example describes the impact of miRNA target sites in circular RNAs on expression levels. Briefly, circular RNA constructs were generated with IRES elements operably linked to a human erythropoietin (hEPO) coding sequence, where 2 tandem miR-122 target sites were inserted into the construct. miR-122-expressing Huh7 cells were transfected with the circular RNAs. hEPO expression in supernatant was assessed 24 and 48 hours after transfection by sandwich ELISA.

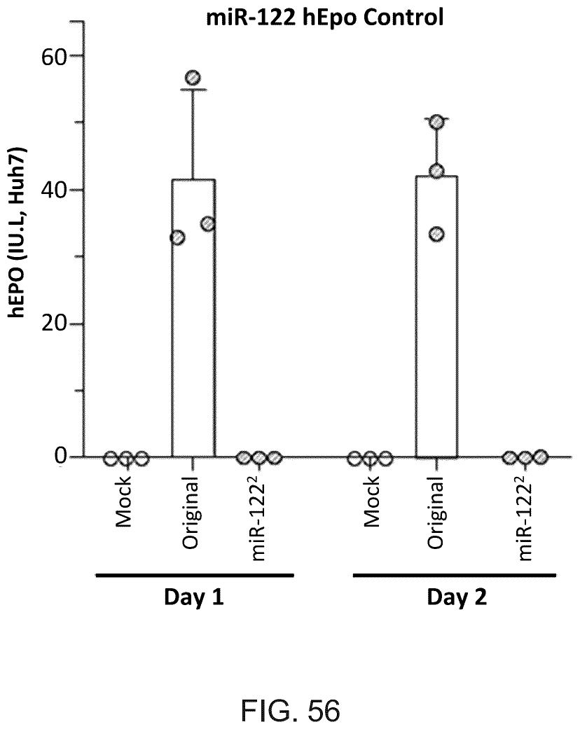

As shown in FIG. 56, the hEPO expression level was abrogated where the miR-122 target sites were inserted into the circular RNA. This result demonstrates that expression from circular RNA can be regulated by miRNA. As such, cell type- or tissue-specific expression can be achieved by incorporating target sites of the miRNAs expressed in the cell types in which expression of the recombinant protein is undesirable.

### Example 50

This example shows immune cell expression of mOX40L circRNA in wildtype mice.

C57BL/6 mice (female, 6-8 weeks, n=3 or 4 per group) were injected intravenously with 0.5 mg/kg mOX40L circRNA LNPs or PBS. After 24 hours, mice were euthanized and their spleens were collected and manually processed into single cell suspensions. Splenocytes were stained for dead cells (LiveDead Fixable Aqua, Thermo) and with anti-mouse antibodies (TCR-B chain, PacBlue, H57-597; CD11b, FITC, ICRF44; B220, PE, RA3-6B2; CD45, PerCP, 30-F11; mOX40L, AF647, RM134L; NK1.1, APC/750, PK136; TruStain FcX, 93; all antibodies from Biolegend) at 1:200. Flow cytometry was performed using an Attune NxT Flow Cytometer (Thermo).

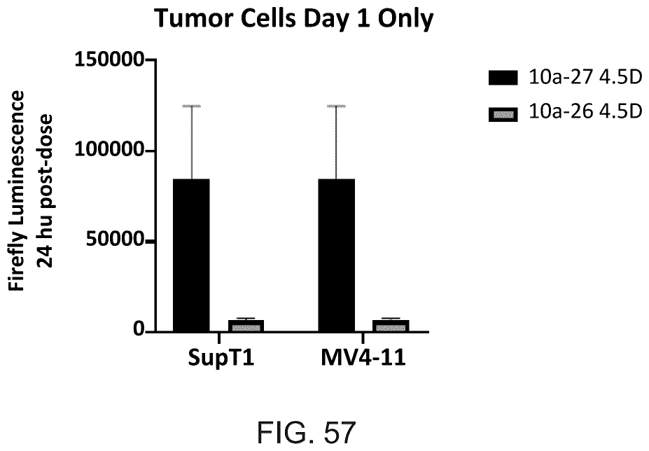

The percent of mOX40L+ cells in splenic myeloid (CD11b+), T cells (TCR-B+), and NK cells (NK1.1+) is presented in FIG. 57. Notably, significantly different transfection efficiencies are observed between the same formulations injected intravenously in different buffers (hypotonic PBS, isotonic PBS, and isotonic TBS). 10a-27 4.5D LNP in hypotonic PBS results in approximately 14% myeloid cell transfection, 6% T cell transfection, and 21% NK cell transfection in the spleen. Of the formulations injected in isotonic buffer, 10a-27 DSPC 5.7A LNP demonstrates myeloid, T cell, and NK cell transfection in the spleen (9%, 3%, and 8%, respectively).

### Example 51

This example shows circRNA-LNP CAR-mediated B cell depletion in mice.

C57BL/6 mice (female, 6-8 weeks, n=5 per group) were injected intravenously with 0.5 mg/kg aCD19-CAR circRNA LNPs or control FLuc circRNA LNPs on Days 0, 2, 5, 7, and 9. On Days −1, 1, 8 and 12, submandibular bleeds were performed to collect blood. 30 μL of blood was lysed with ACK lysis buffer and washed with MACS buffer to isolate immune cells. On Day 12, mice were euthanized and their spleens were collected and manually processed into single cell suspensions. To assess the frequency of B cells in the blood and spleen, these single cell suspensions were stained for dead cells (LiveDead Fixable Aqua, Thermo) and stained with anti-mouse antibodies (TCR-B chain, PacBlue, H57-597; CD11b, FITC, ICRF44; B220, PE, RA3-6B2; CD45, PerCP, 30-F11; TruStain FcX, 93; all antibodies from Biolegend) at 1:200. Flow cytometry was performed using an Attune NxT Flow Cytometer (Thermo).

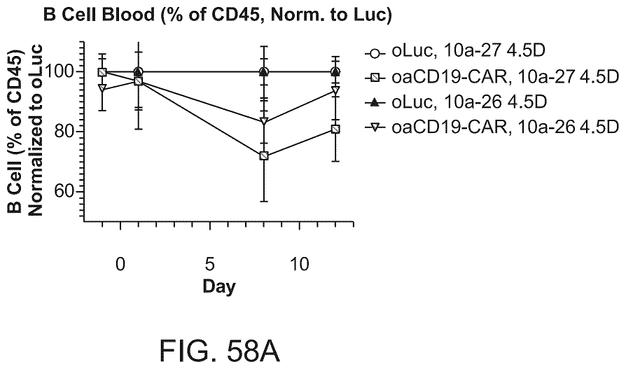

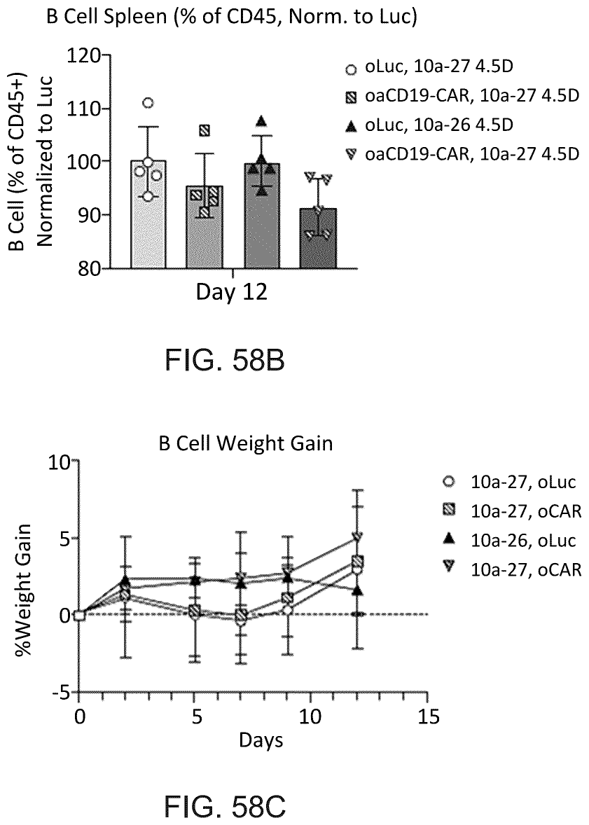

FIG. 58A quantifies the B cell depletion observed in this study, as defined by percentage of B220+ B cells of live, CD45+ immune cells. The B cell depletion in the aCD19-CAR circRNA LNP group was compared to its respective FLuc circRNA LNP control on Days 8 and 12 (for blood) and Day 12 (for spleen). In the blood, aCD19-CAR 10a-27 (4.5D) and 10a-26 (4.5D) LNPs resulted in approximately 28% and 17% reductions, respectively, in % B220+ of live CD45+ at Day 8 compared to FLuc control. In the spleen, aCD19-CAR 10a-27 (4.5D) and 10a-26 (4.5D) LNPs resulted in approximately 5% and 9% reductions in % B220+ of live CD45+ at Day 12 compared to FLuc control as shown in FIG. 58B. In all, these results suggest that CAR-mediated B cell depletion is occurring in mice treated with aCD19-CAR circRNA LNPs for Lipid 10a-27 (4.5D) and Lipid 10a-26 (4.5D).

In addition, FIG. 58C shows the percent weight gain of mice in this study. There was not significant weight loss on average from the 10a-27 4.5D or 10a-26 4.5D LNP treated mice (5×0.5 mg/kg over 9 days), suggesting that these LNPs may be well-tolerated in mice at this dose and schedule.

TABLE 3
H. Characterization of LNPs

| | Z-Average | | RNA Encapsulation |
|---|---|---|---|
| Formulation | (nm) | PDI | Efficiency (%) |
| 10a-27 (4.5D), oLuc | 65 | 0.03 | 93 |
| 10a-27 (4.5D), oaCD19-CAR | 74 | 0.02 | 96 |
| 10a-26 (4.5D), oLuc | 71 | 0.04 | 91 |
| 10a-26, (4.5D), oaCD19-CAR | 71 | 0.04 | 93 |

### Example 52

### LNP and Circular RNA Construct Containing Anti-CD19 CAR Reduces B Cells in the Blood and Spleen In Vivo.

Circular RNA constructs encoding an anti-CD19 CAR expression were encapsulated within lipid nanoparticles as described above. For comparison, circular RNAs encoding luciferase expression were encapsulated within separate lipid nanoparticle.

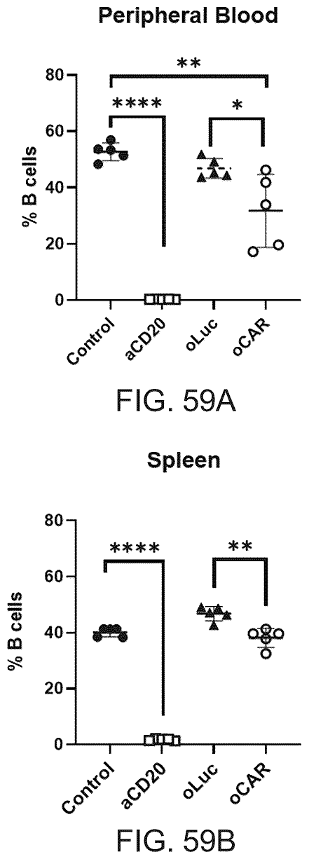

C57BL/6 mice at 6 to 8 weeks old were injected with either LNP solution every other day for a total of 4 LNP injections within each mouse. 24 hours after the last LNP injection, the mice's spleen and blood were harvested, stained, and analyzed via flow cytometry. As shown in FIG. 59A and FIG. 59B, mice containing LNP-circular RNA constructs encoding an anti-CD19 CAR led to a statistically significant reduction in CD19+ B cells in the peripheral blood and spleen compared to mice treated with LNP-circular RNA encoding a luciferase.

### Example 53

IRES Sequences Contained within Circular RNA Encoding CARs Improves CAR Expressions and Cytotoxicity of T-Cells.

Activated murine T-cells were electroporated with 200 ng of circular RNA constructs containing a unique IRES and a murine anti-CD19 1D3ζ CAR expression sequence. The IRES contained in these constructs were derived either in whole or in part from a Caprine Kobuvirus, Apodemus Picornavirus, Parabovirus, or Salivirus. A Caprine Kobuvirus derived IRES was additionally codon optimized. As a control, a circular RNA containing a wild-type zeta mouse CAR with no IRES was used for comparison. The T-cells were stained for the CD-19 CAR 24 hours post electroporation to evaluate for surface expression and then co-cultured with A20 Fluc target cells. The assay was then evaluated for cytotoxic killing of the Fluc+A20 cells 24 hours after co-culture of the T-cells with the target cells.

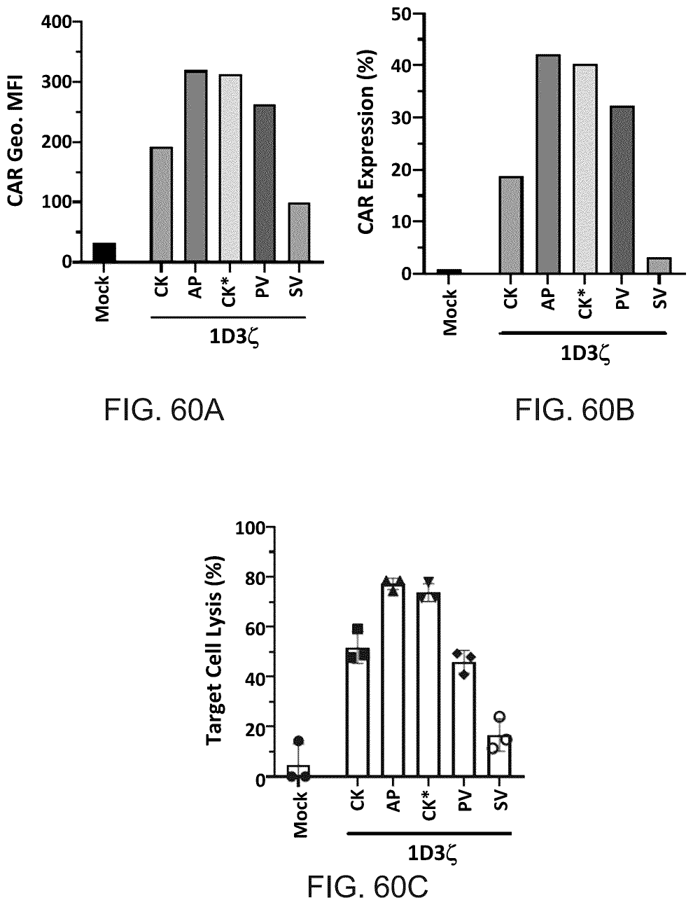

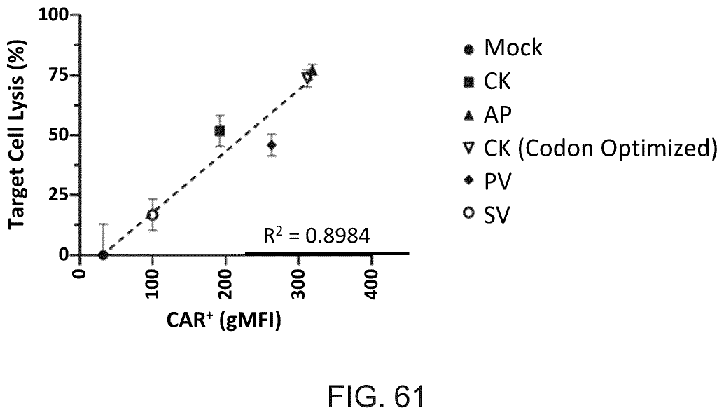

As seen in FIGS. 60A, 60B, 60C, and 61, the unique IRES were able to increase the frequency that the T-cells expressed the CAR protein and level of CAR expression on the surface of the cells. The increased frequency of expression of the CAR protein and level of CAR expression on the surface of cells lead to an improved anti-tumor response.

### Example 54

Cytosolic and Surface Proteins Expressed from Circular RNA Construct in Primary Human T Cells.

Circular RNA construct contained either a sequence encoding for a fluorescent cytosolic reporter or a surface antigen reporter. Fluorescent reporters included green fluorescent protein, mCitrine, mWasabi, Tsapphire. Surface reporters included CD52 and Thy1.1^{bio}. Primary human T cells were activated with an anti-CD3/anti-CD28 antibody and electroporated 6 days post activation of the circular RNA containing a reporter sequence. T cells were harvested and analyzed via flow cytometry 24 hours post electroporation. Surface antigens were stained with commercially available antibodies (e.g., Biolegend, Miltenyi, and BD).

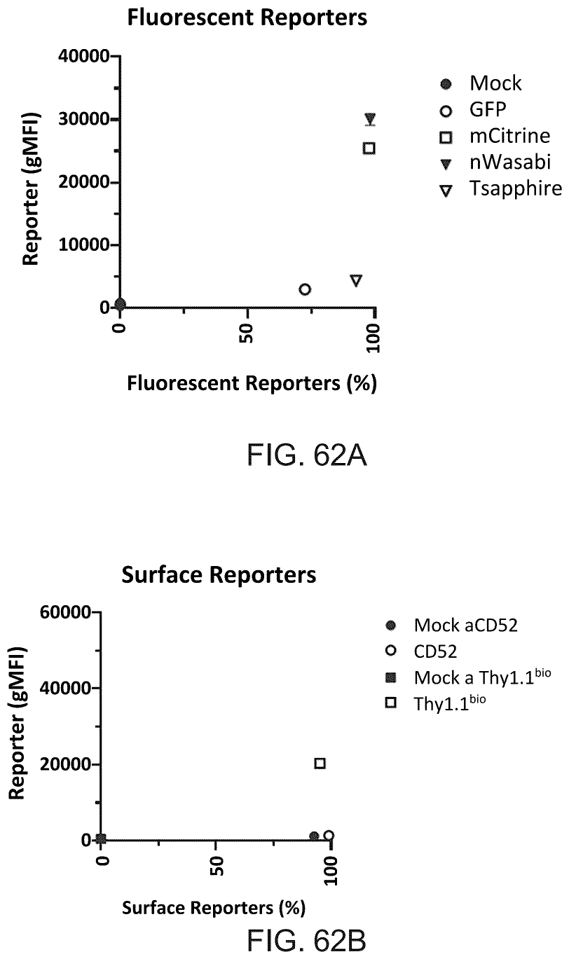

As seen in FIG. 62A and FIG. 62B, cytosolic and surface proteins can be expressed from circular RNA encoding the proteins in primary human T cells.

### Example 55

Circular RNAs Containing Unique IRES Sequences have Improved Translation Expression Over Linear mRNA.

Circular RNA constructs contained a unique IRES along with an expression sequence for Firefly luciferase (FLuc).

Human T cells from 2 donors were enriched and stimulated with anti-CD3/anti-CD28 antibodies. After several days of proliferation, activated T cells were harvested and electroporated with equal molar of either mRNA or circular RNA expressing FLuc payloads. Various IRES sequences, including those derived from Caprine Kobuvirus, Apodemus Picornavirus, and Parabovirus, were studied to evaluate expression level and durability of the payload expression across 7 days. Across the 7 days, the T cells were lysed with Promega Brightglo to evaluate for bioluminescence.

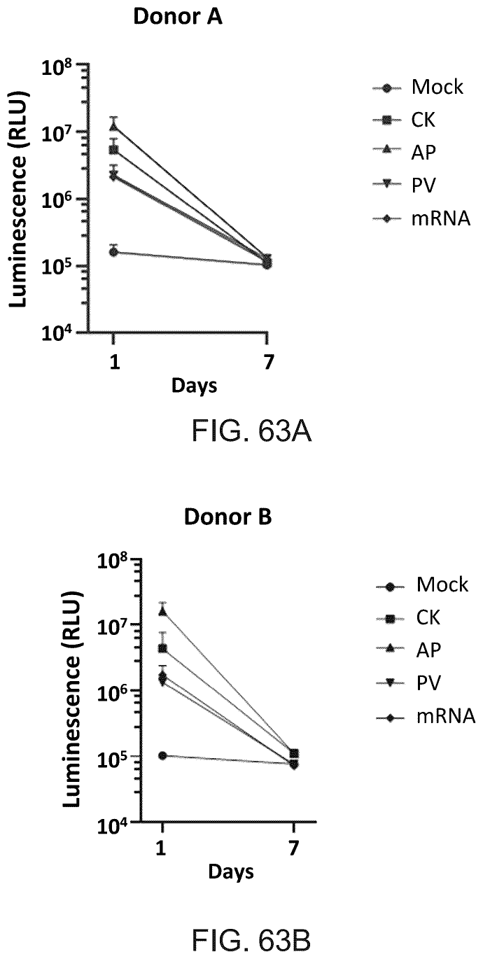

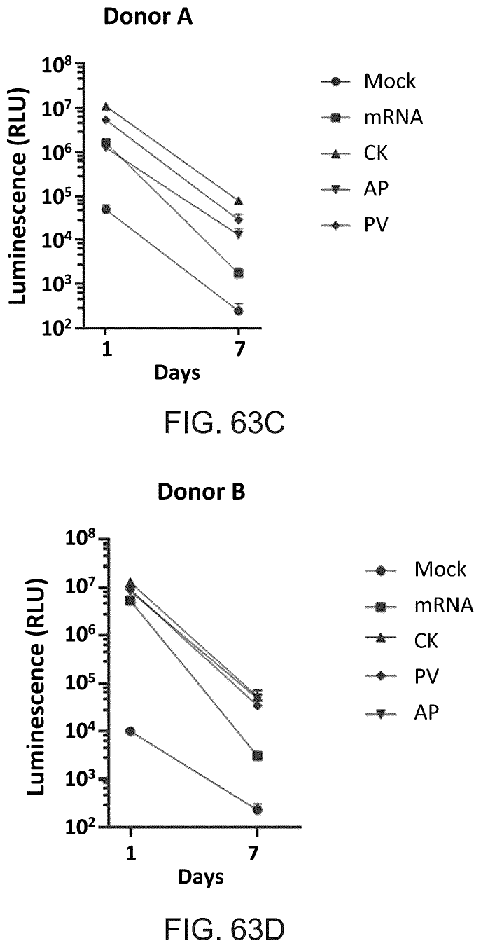

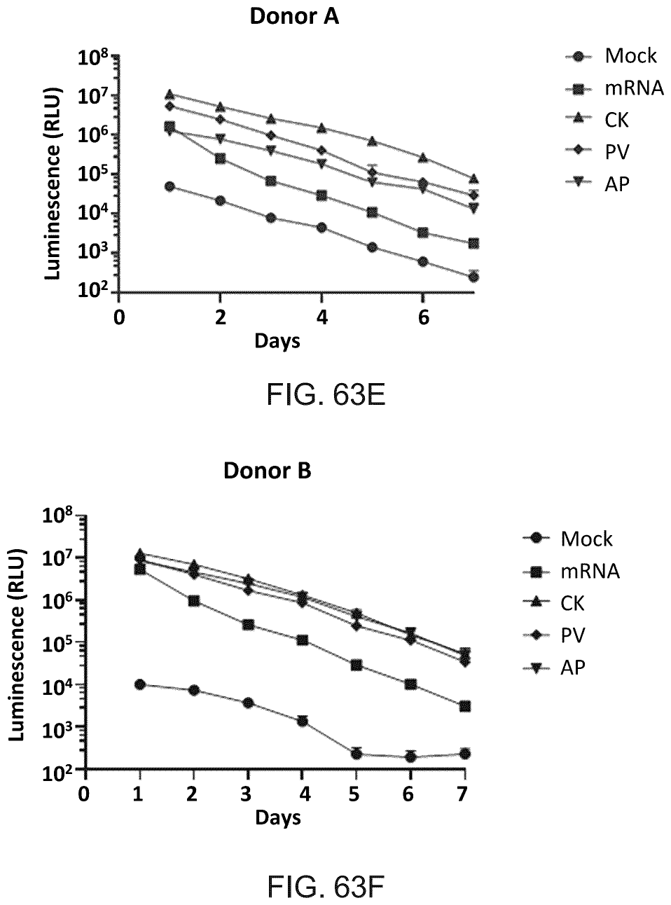

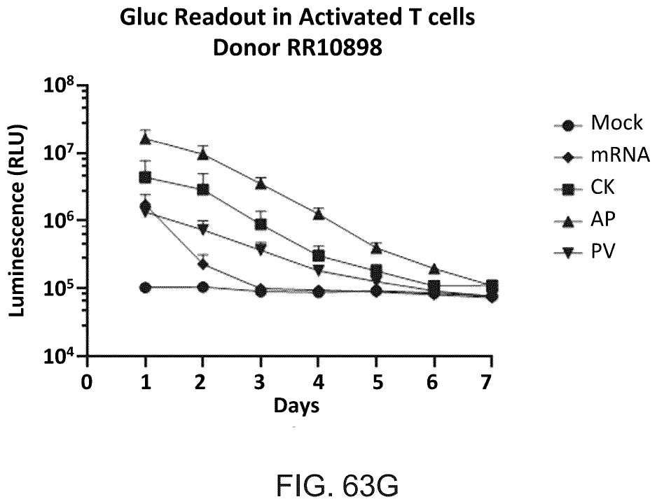

As shown in FIGS. 63C-63G, the presence of an IRES within a circular RNA can increase translation and expression of a cytosolic protein by orders of magnitude and can improve expression compared to linear mRNA. This was found consistent across multiple human T cell donors.

### Example 56

### Example 56A: LNP-Circular RNA Encoding Anti-CD19 Mediates Human T Cell Killing of K562 Cells

Circular RNA constructs contained a sequence encoding for anti-CD19 antibodies. Circular RNA constructs were then encapsulated within a lipid nanoparticle (LNP).

Human T cells were stimulated with anti-CD3/anti-CD28 and left to proliferate up to 6 says. At day 6, LNP-circular RNA and ApoE3 (1 μg/mL) were co-cultured with the T cells to mediate transfection. 24 hours later, Fluc+K562 cells were electroporated with 200 ng of circular RNA encoding anti-CD19 antibodies and were later co-cultured at day 7. 48 hours post co-culture, the assay was assessed for CAR expression and cytotoxic killing of K562 cells through Fluc expression.

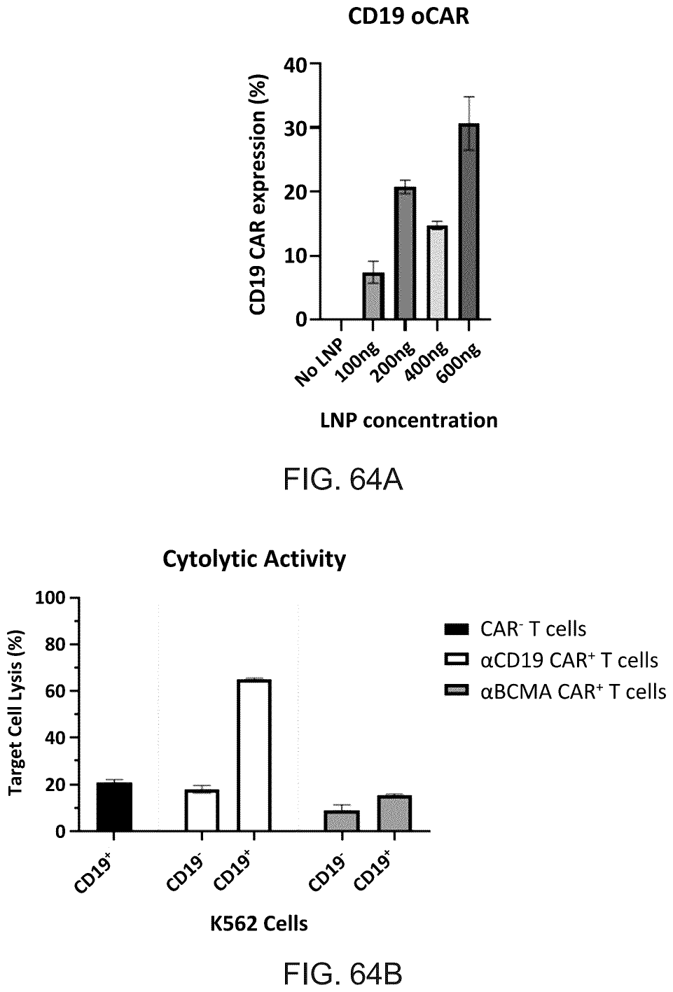

As shown in FIG. 64A and FIG. 64B, there is T cell expression of anti-CD19 CAR from the LNP-mediated delivery of a CAR in vitro to T cells and its capability to lyse tumor cells in a specific, antigen dependent manner in engineered K562 cells.

### Example 56B: LNP-Circular RNA Encoding Anti-BCMA Antibody Mediates Human T Cell Killing of K562 Cells

Circular RNA constructs contained a sequence encoding for anti-BCMA antibodies. Circular RNA constructs were then encapsulated within a lipid nanoparticle (LNP).

Human T cells were stimulated with anti-CD3/anti-CD28 and left to proliferate up to 6 says. At day 6, LNP-circular RNA and ApoE3 (1 μg/mL) were co-cultured with the T cells to mediate transfection. 24 hours later, Fluc+K562 cells were electroporated with 200 ng of circular RNA encoding anti-BCMA antibodies and were later co-cultured at day 7. 48 hours post co-culture, the assay was assessed for CAR expression and cytotoxic killing of K562 cells through Fluc expression.

As shown in FIG. 64B, there is T cell expression of BCMA CAR from the LNP-mediated delivery of a CAR in vitro to T cells and its capability to lyse tumor cells in a specific, antigen dependent manner in engineered K562 cells.

### Example 57

### Anti-CD19 CAR T Cells Exhibit Anti-Tumor Activity In Vitro.

Human T cells were activated with anti-CD3/anti-CD28 and electroporated once with 200 ng of anti-CD19 CAR-expressing circular RNA. Electroporated T cells were co-cultured with FLuc+ Nalm6 target cells and non-target Fluc+K562 cells to evaluate CAR-mediated killing. After 24 hours post co-culture, the T cells were lysed and examined for remanent FLuc expression by target and non-target cells to evaluate expression and stability of expression across 8 days total.

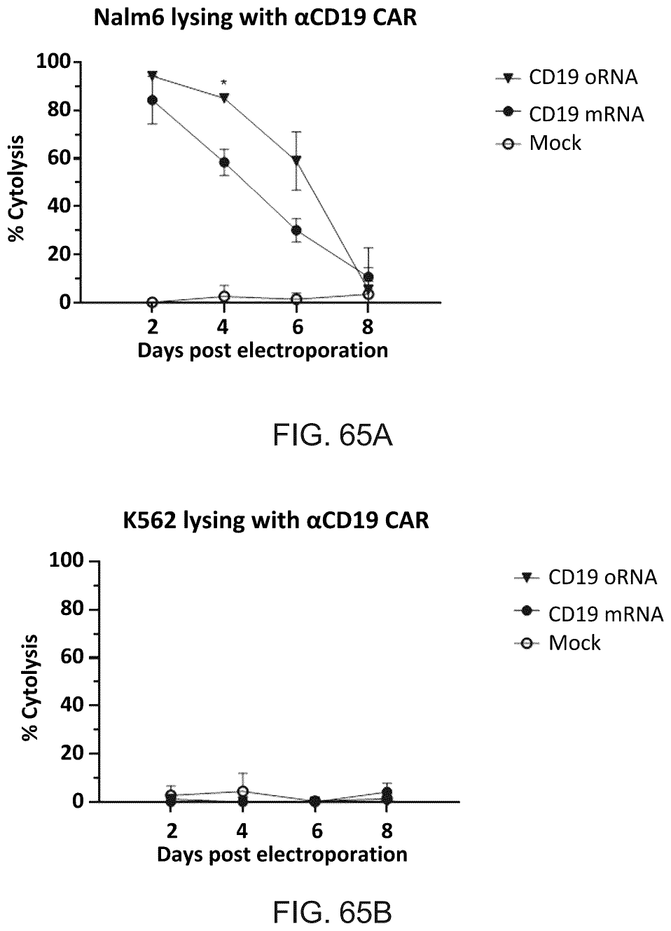

As shown in FIGS. 65A and 65B, T cells express circular RNA CAR constructs in specific, antigen-dependent manner. Results also shows improved cytotoxicity of circular RNAs encoding CARs compared to linear mRNA encoding CARs and delivery of a functional surface receptor.

### Example 58

Effective LNP Transfection of Circular RNA Mediated with ApoE3

Human T cells were stimulated with anti-CD3/anti-CD28 and left to proliferate up to 6 days. At day 6, lipid nanoparticle (LNP) was and circular RNA expressing green fluorescence protein solution with or without ApoE3 (1 μg/mL) were co-cultured with the T cells. 24 hours later, the T cells were stained for live/dead T cells and the live T cells were analyzed for GFP expression on a flow cytometer.

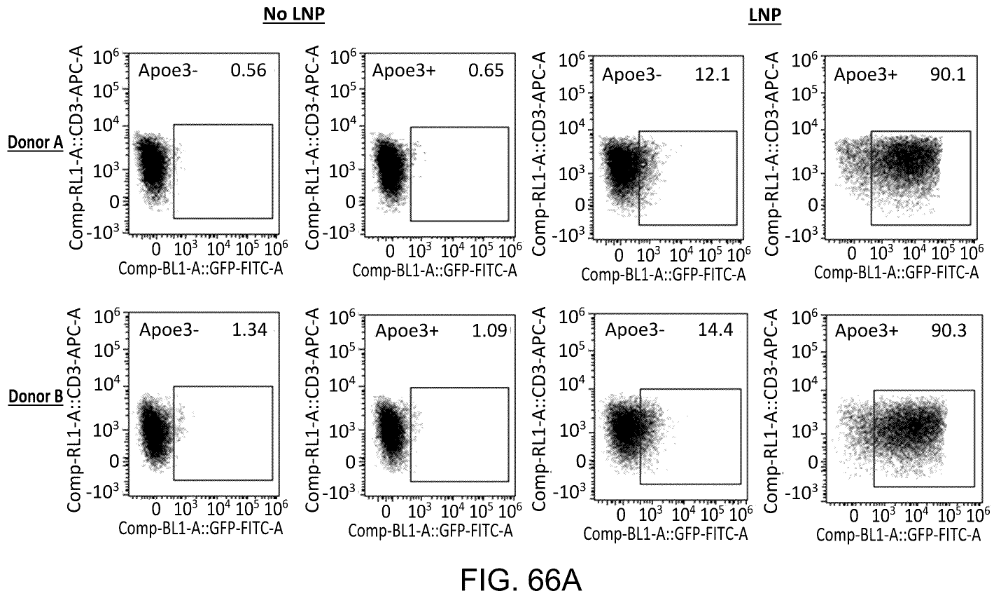

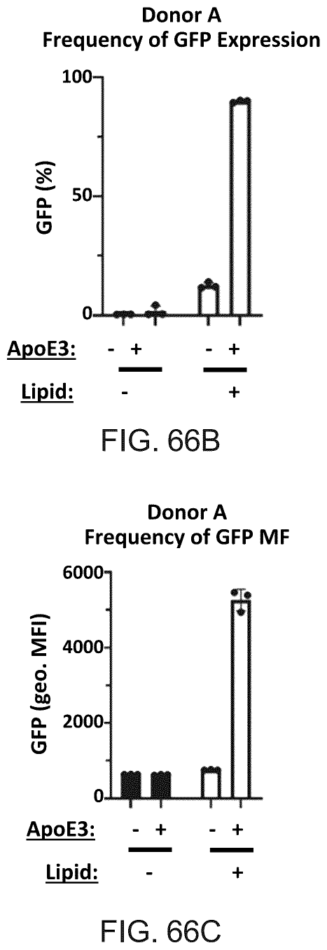

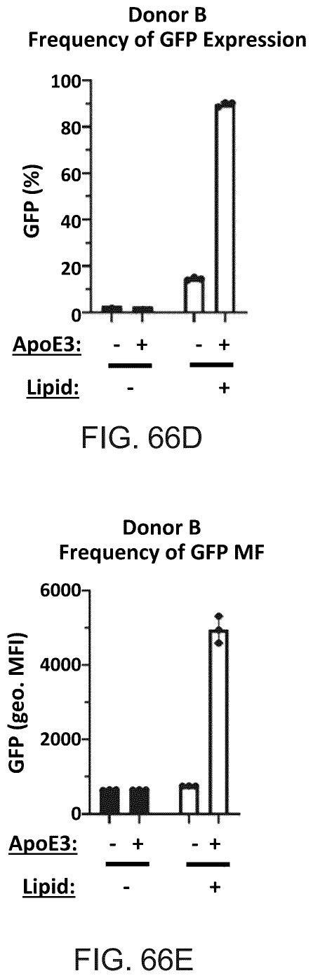

As shown by FIGS. 66A-66E, efficient LNP transfection can be mediated by ApoE3 on activated T cells, followed by significant payload expression. These results were exhibited across multiple donors.

### Example 59

This example shows in vivo cytokine response after IV injection of 0.2 mg/kg circRNA preparations encapsulated in a lipid nanoparticle formulation. circRNA splicing reactions synthesized with GTP as a precursor RNA initiator and splicing nucleotide incited greater cytokine responses than purified circRNA and linear mlyI-mRNA due to the presence of triphosphorylated 5′ termini in the splicing reaction. Levels of IL--10, IL-6, IL-10, IL-12p70, RANTES, TNFα were measured from blood drawn 6 hours following intravenous injection of the LNP-formulated circRNA preparation. Mice injected with PBS were used as a control.

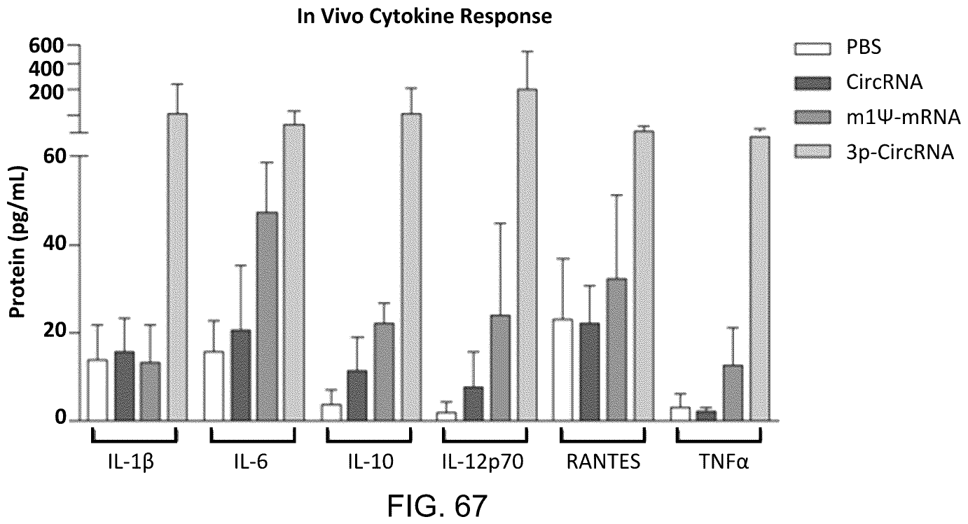

As seen in FIG. 67, circRNA splicing reactions synthesized with GTP as a precursor RNA initiator and splicing nucleotide incite greater cytokine responses than purified circRNA and linear mlW-mRNA due to the presence of triphosphorylated 5′ termini in the splicing reaction.

### Example 60

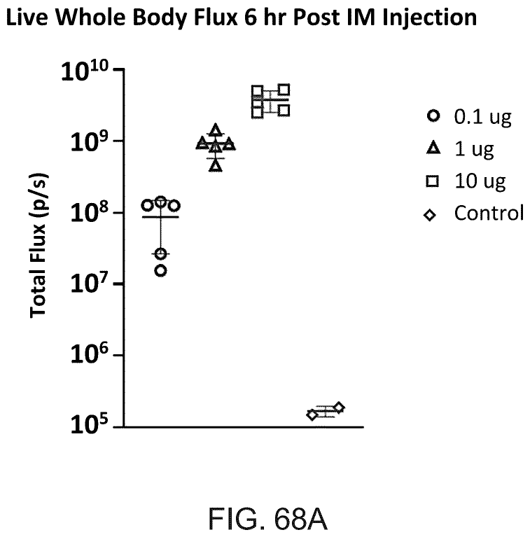

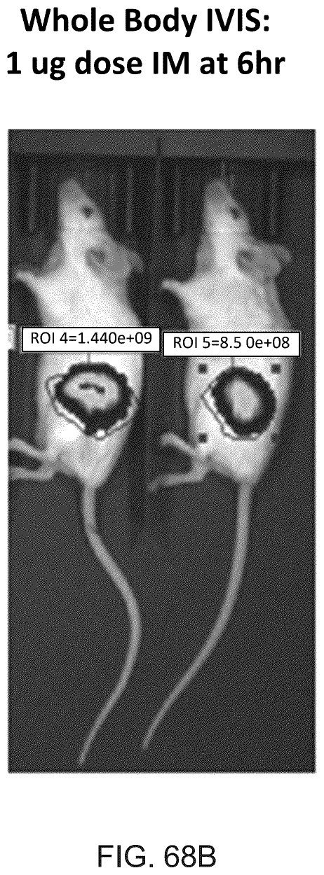

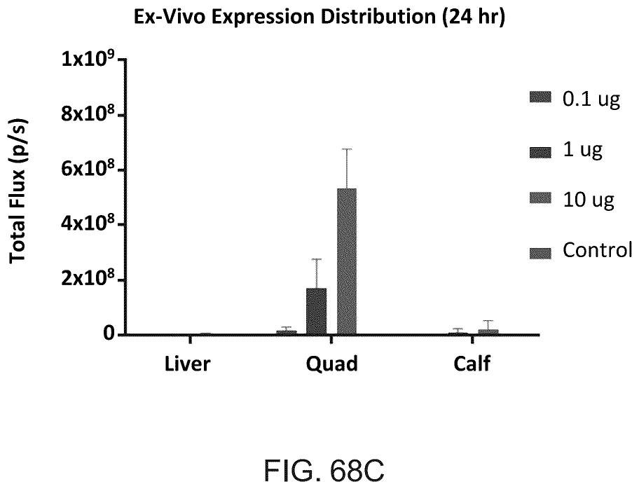

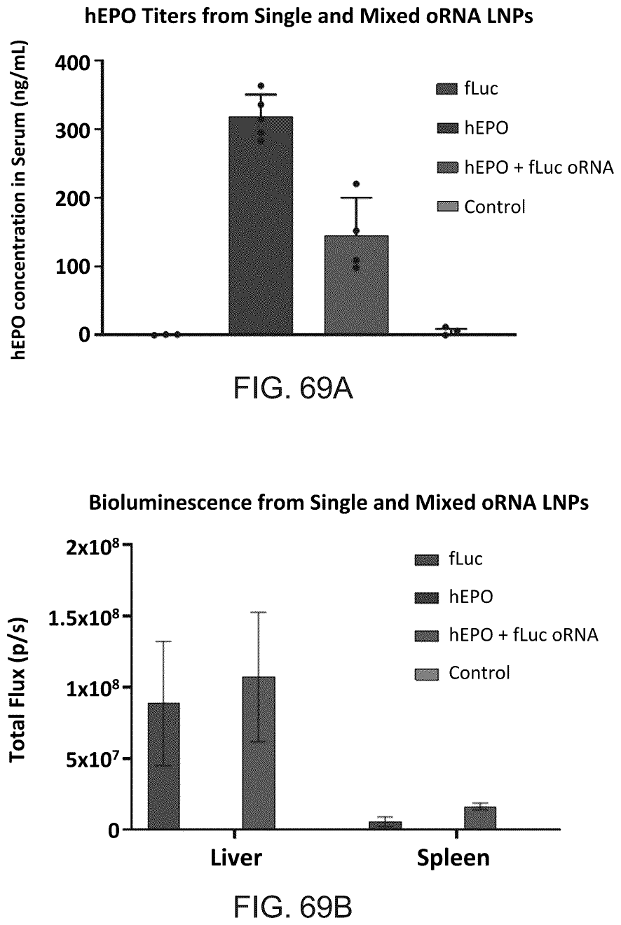

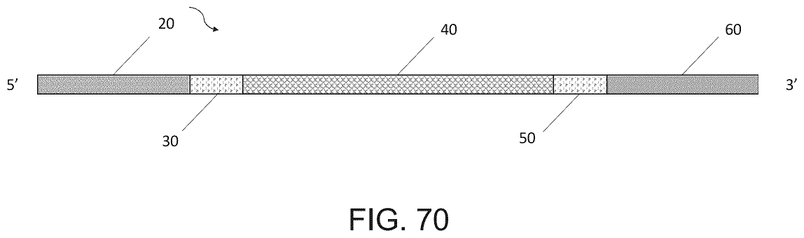

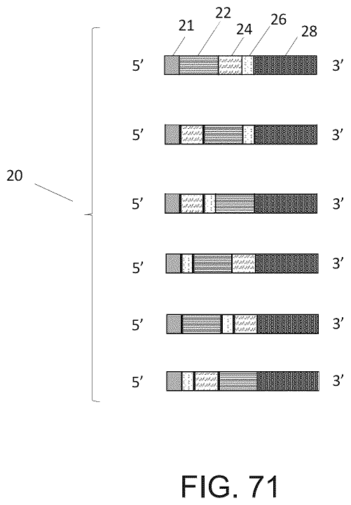

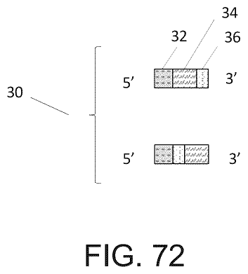

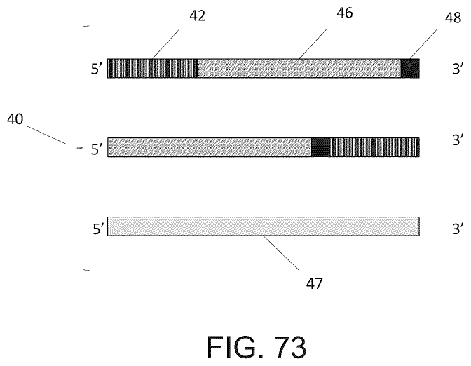

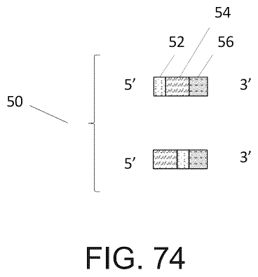

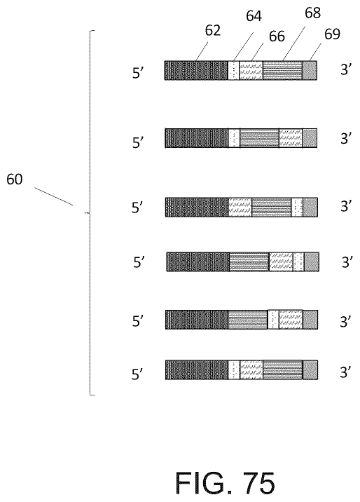

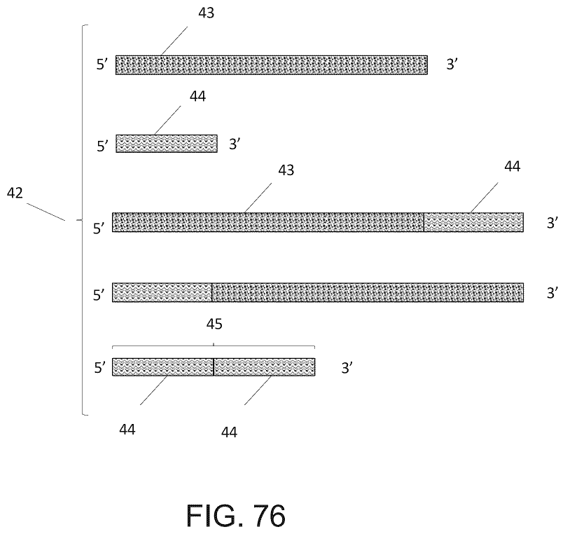

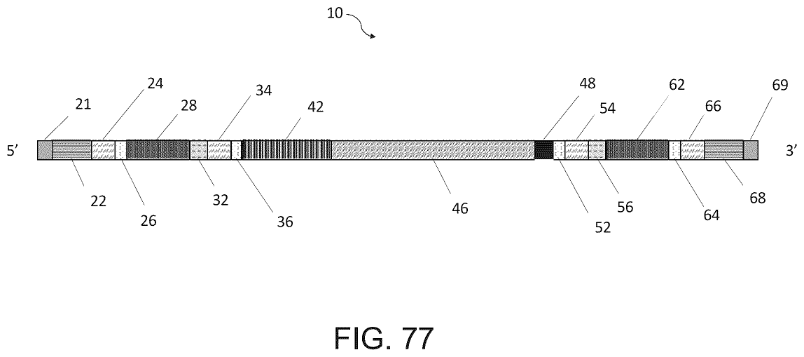

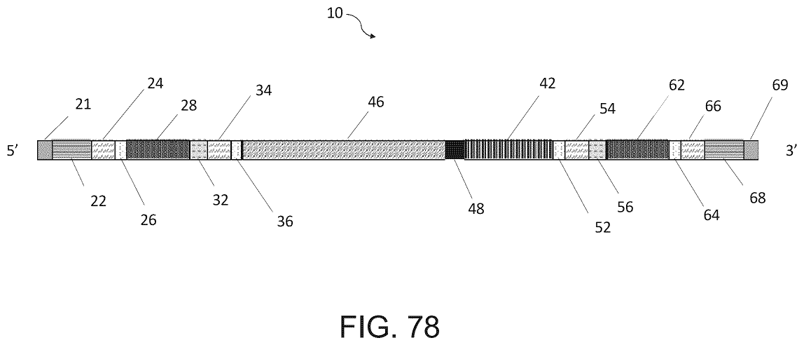

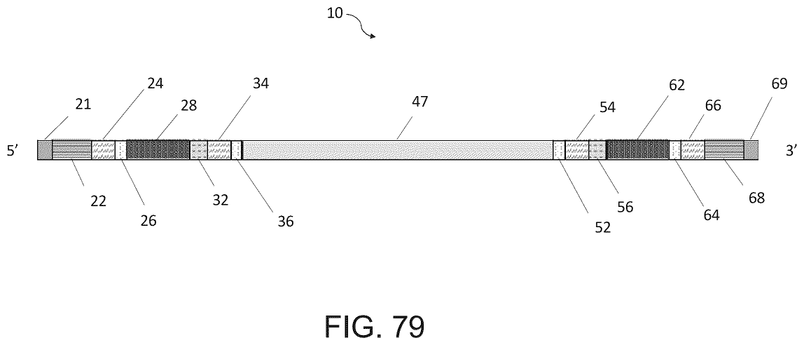

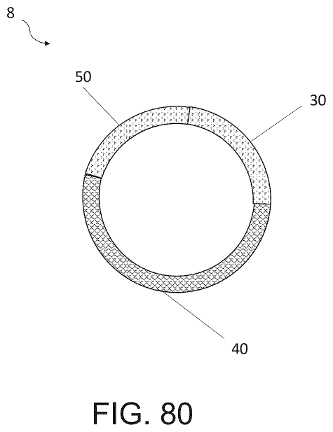

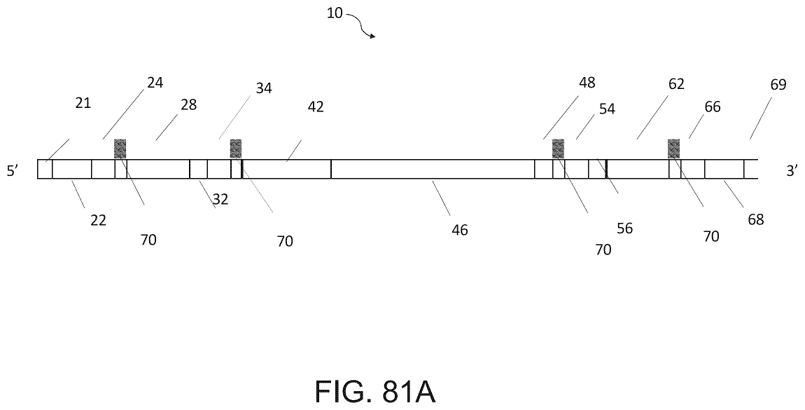

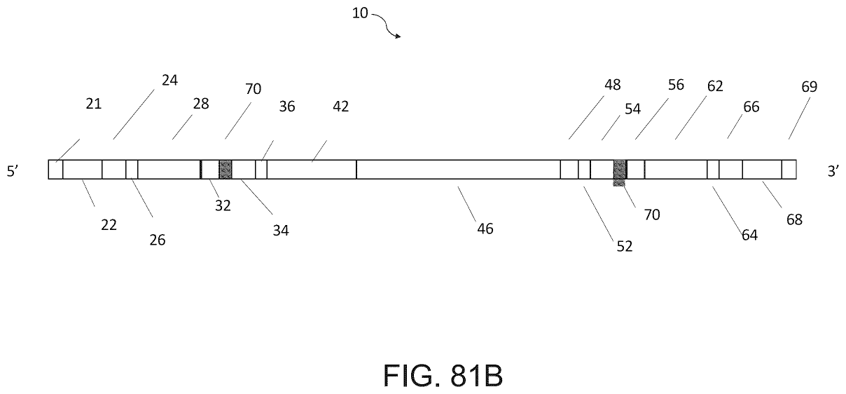

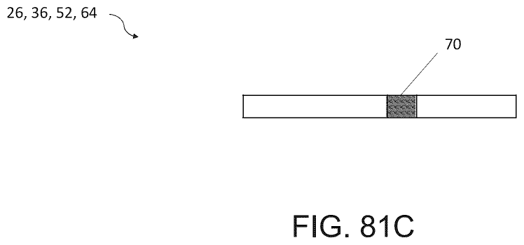

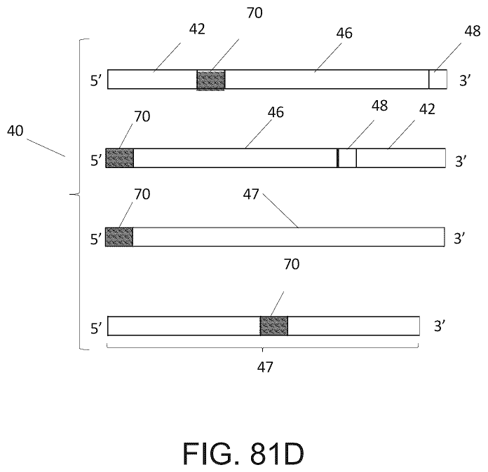

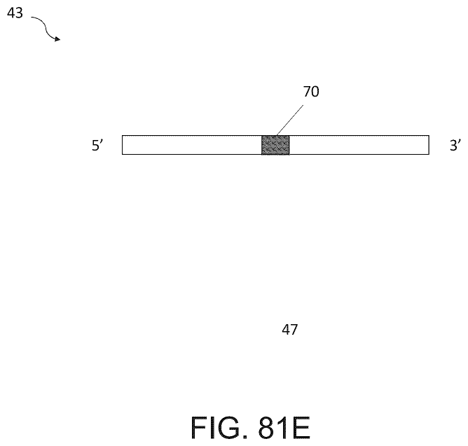

This example illustrates intramuscular delivery of varying doses of lipid nanoparticle containing circular RNAs. Mice were dosed with either 0.1 μg, 1 μg, or 10 μg of circRNA formulated in lipid nanoparticles. Whole body IVIS imagine was conducted at 6 hours following an injection of luciferin (FIG. 68A and FIG. 68B). Ex vivo IVIS imaging was conducted at 24-hour. Flux values for liver, quad, and calf are shown in FIG. 68C. FIG. 69B and FIG. 69C show that the expression of the circular RNA is present in the muscle tissue of the mice.

### Example 61

This example illustrates expression of multiple circular RNAs in LNP formulations. Circular RNA constructs encoding either hEPO or fLuc were dosed in a single and mixed set of LNPs. hEPO concentration in the serum (FIG. 69A) and total flux by IVIS imaging (FIG. 69B) was determined. The results show that the circular RNA hEPO or fLuc constructs individually formulated or co-formulated expressed protein efficiently.

### Example 62

### Example 62A: Hepatocyte Plating and Culture

Primary human hepatocytes (PHH), primary mouse hepatocytes (PMH), primary cynomolgus monkey hepatocytes (PCH) were thawed and resuspended in hepatocyte thawing medium (Xenotech, cat #K8600/K8650) followed by centrifugation. The supernatant was discarded, and the pelleted cells were resuspended in hepatocyte plating medium (Xenotech, cat #K8200). Cells were counted via hemocytometer and plated on Bio-coat collagen-I coated 96-well plates at a density of 25,000 cells/well for PHH, 25,000 cells/well for PMH, and 50,000 cells/well for PCH in 100 μL of plating media. Plated cells were allowed to settle and adhere for 6 hours in a tissue culture incubator at 37° C. and 5% CO₂ atmosphere. After incubation cells were checked for monolayer formation after which the plating media was aspirated and replaced with 100 ul of culture media (Xenotech, cat #K8300). Media was replaced every 24 hours for the duration of the experiment.

### Example 62B: In Vitro Screening of LNP Formulated Circular RNA Encoding Firefly Luciferase in Primary Human, Mouse, and Cynomolgus Monkey Hepatocytes

A circular RNA construct comprising a TIE and a coding element encoding for firefly luciferase was produced and transfected into LNPs. Various concentrations of LNPs formulated with the circularized RNA (oRNA) were diluted in hepatocyte media supplemented with 3% fetal bovine serum (FBS) (ThermoFisher, cat #A3160401). Media was aspirated from the cells prior to addition of 100 μL of LNP/FBS/media mixture to the cells.

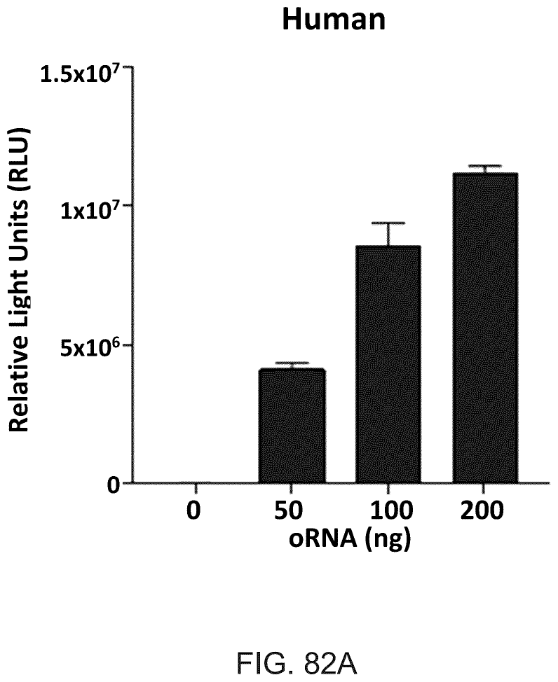

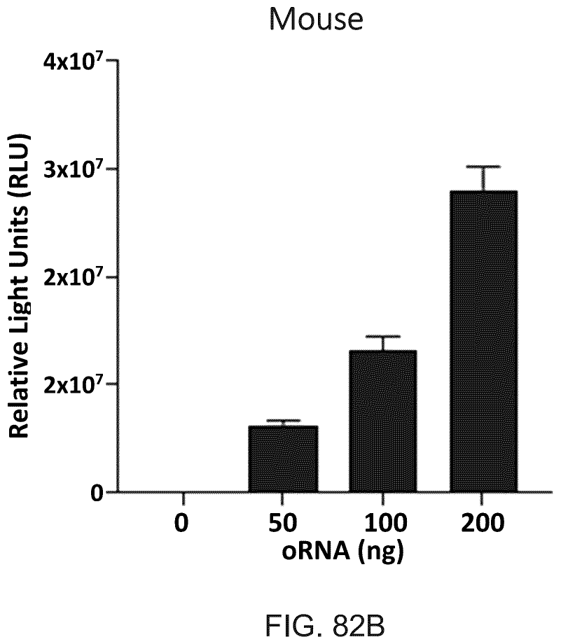

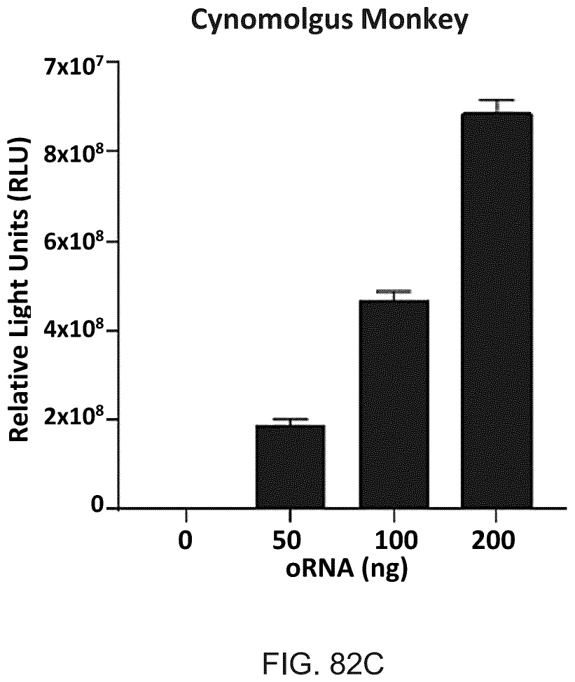

Luciferase activity was detected in primary human (FIG. 82A), mouse (FIG. 82B), and cynomolgus monkey (FIG. 82C) hepatocyte. 24 hours post-transfection plates were removed from the incubator and allowed to equilibrate to room temperature for 15 mins. A volume of 100 μL of Firefly Luciferase one-step glow assay working solution (Pierce, cat #16196) was added to each well. The plate was placed on a microplate shaker (ThermoFisher, cat #S72050) and mixed at 300 rpm for 3 min. Post-mixing, the plate was allowed to incubate at room temperature for 10 min. Luminescence was read using a Varioskan or Bio-Tek Cytation5 instrument.

As seen in FIG. 82A, FIG. 82B, and FIG. 82C, TIE containing circular RNAs are capable of driving firefly luciferase protein expression in primary hepatocytes from multiple species in a dose-dependent manner when transfected in vitro with an LNP.

### Example 62C: In Vitro Screening of LNP Formulated Circular RNA Encoding Firefly Luciferase in Multiple Primary Human Hepatocyte Donors

A circular RNA construct comprising a TIE and a coding element encoding for firefly luciferase was produced and transfected into LNPs. Various concentrations of LNPs formulated with the circularized RNA (oRNA) were diluted in hepatocyte media supplemented with 3% fetal bovine serum (FBS) (ThermoFisher, cat #A3160401). Media was aspirated from the cells prior to addition of 100 ul of LNP/FBS/media mixture to the cells.

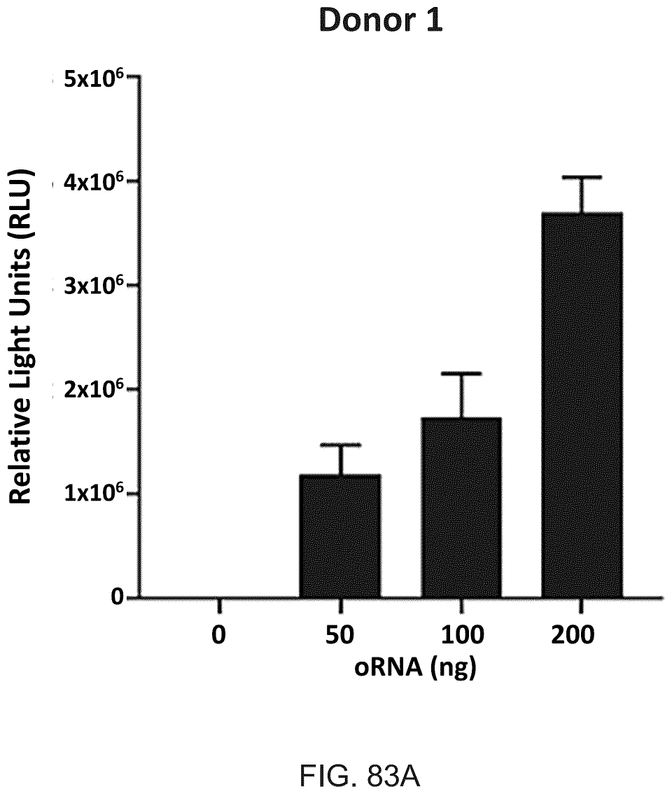

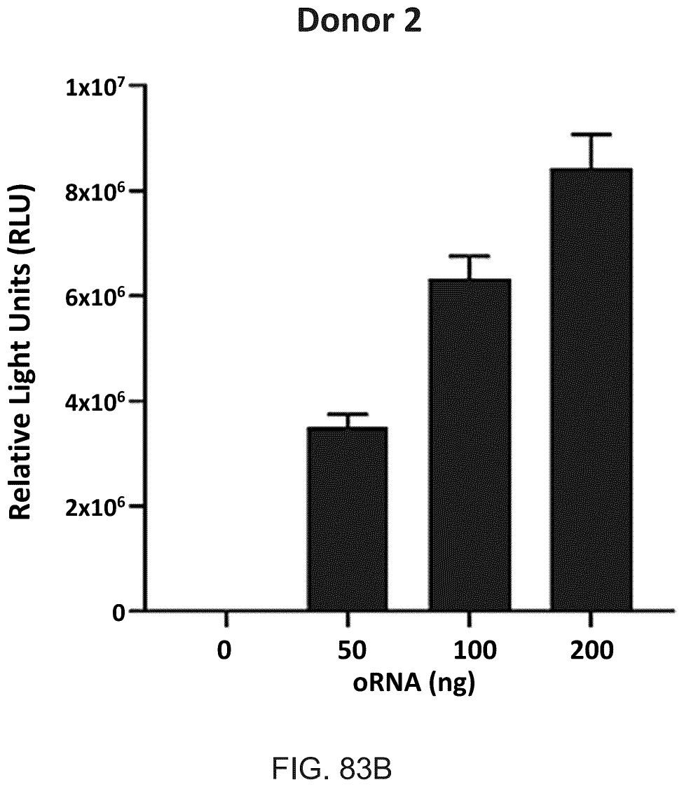

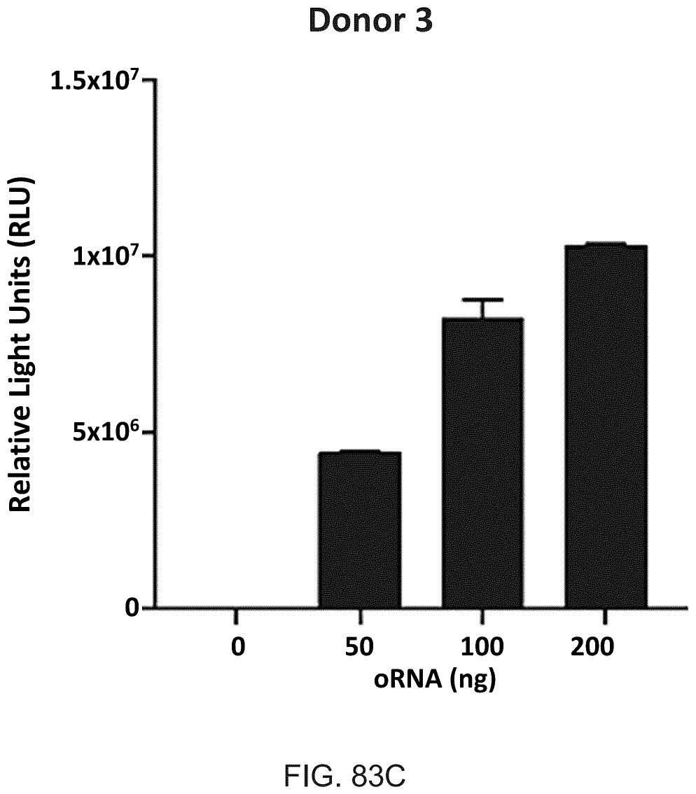

Luciferase activity was detected in primary human (FIG. 83A), mouse (FIG. 83B), and cynomolgus monkey (FIG. 83C) hepatocyte. 24 hours post-transfection plates were removed from the incubator and allowed to equilibrate to room temperature for 15 mins. A volume of 100 μL of Firefly Luciferase one-step glow assay working solution (Pierce, cat #16196) was added to each well. The plate was placed on a microplate shaker (ThermoFisher, cat #S72050) and mixed at 300 rpm for 3 min. Post-mixing, the plate was allowed to incubate at room temperature for 10 min. Luminescence was read using a Varioskan or Bio-Tek Cytation5 instrument.

As seen in FIG. 83A-83C, TIE containing circular RNAs are capable of driving firefly luciferase protein expression in primary hepatocytes from multiple human donors in a dose-dependent manner when transfected in vitro with an LNP.

### Example 63

In Vitro Expression of LNP Formulated with Circular RNA Encoding for GFP in Multiple Human Cell Models.

A circular RNA construct was produced comprising a TIE and coding element encoding for a GFP protein. LNP were formulated with the circular RNA construct. Then various concentrations of the LNP containing the circular RNA construct were diluted in hepatocyte media supplemented with 3% fetal bovine (FBS) (ThermoFisher, cat #A3160401). Media was aspirated from the cells prior to addition of 100 ul of LNP/FBS/media mixture to the cells.

HeLa (human cervical adenocarcinoma; ATCC, cat #CCL-2), HEK293 (human embryonic kidney; ATCC, cat #CRL-1573), and HUH7 (human liver hepatocellular carcinoma; JCRB, cat #JCRB0403) were transfected as previously described with LNP formulated oRNAs. Twenty-four hours post-transfection, the media was removed and the cells were trypsinized. The trypsinized cells were neutralized with PBS supplemented with 10% FBS, harvested, and transferred to a tube. The tube was centrifuged to pellet the cells and the supernatant was aspirated. The pellet was stored at −80° C. prior to lysis. For lysis the cells were thawed on ice and were lysed with 100 μL/well RIPA buffer (Boston Bio Products, Cat. BP-115) plus freshly added 1 mM DTT, and 250 U/mL Benzonase (EMD Millipore, cat #71206-3), and protease inhibitor mixture consisting of complete protease inhibitor cocktail (Sigma, cat #11697498001). Cells were kept on ice for 30 minutes at which time NaCl (1M final concentration) was added. Cell lysates were thoroughly mixed and retained on ice for 30 min. The whole cell extracts (WCE) were centrifuged to pellet debris. A Bradford assay (Bio-Rad, cat #500-0001) was used to assess protein content of the lysates. The Bradford assay procedure was completed according to the manufacturer's protocol. Extracts were stored at −20° C. prior to use. Western blots were performed to assess GFP protein levels. Whole cell extract lysates were mixed with Laemmli buffer and denatured at 95° C. for 10 min. Western blots were run using the NuPage system on 4-12% Bis-Tris gels (ThermoFisher, cat #NPO335BOX) according to the manufacturer's protocol followed by wet transfer onto 0.45 μm nitrocellulose membrane (ThermoFisher, cat #LC2001). After transfer membranes were rinsed thoroughly with water and stained with Ponceau S solution (Boston Bio Products, cat #ST-180) to confirm complete and even transfer. Blots were blocked using 5% Dry Milk in TBS for 30 minutes on a lab rocker at room temperature. Blots were rinsed with 1×TBST (Boston BioProducts, cat #IBB-180) and probed with mouse dylight 680-tagged anti-GFP monoclonal antibody (ThermoFisher, cat #MA515256D680) at 1:1,000 in 1×TBST. Anti-3-actin or GAPDH was used as a loading control (ThermoFisher, cat #AM4302/AM4300) at 1:4,000 in 1× TBST and incubated simultaneously with the GFP primary antibody. Blots were sealed in a bag and kept overnight at 4° C. on a lab rocker. After incubation, blots were rinsed 3 times for 5 minutes each in 1× TBST and probed with mouse secondary antibodies (ThermoFisher, cat #PI35519) at 1:25,000 each in 1× TBST for 30 minutes at room temperature. After incubation, blots were rinsed 3 times for 5 minutes each in 1× TBST. Blots were visualized and analyzed using a Licor Odyssey system.

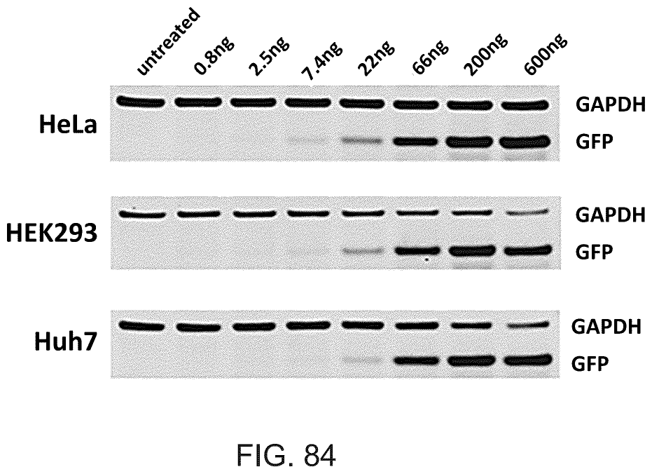

As shown in FIG. 84, TIE-containing circular RNA is capable of expressing GFP protein in diverse human cell lines (e.g., HeLa, HEK293, and HUH7 cells) in a dose dependent manner when transfected in vitro with an LNP.

### Example 64

In Vitro Expression of LNP Formulated with Circular RNA Encoding for GFP in Primary Human Hepatocytes.

A circular RNA construct was produced comprising a TIE and coding element encoding for a GFP protein. Various concentrations of LNP containing circularized RNA (oRNA) were diluted in hepatocyte media supplemented with 3% fetal bovine serum (FBS) (ThermoFisher, cat #A3160401). Media was aspirated from the cells prior to addition of 100 μL of LNP/FBS/media mixture to the cells.

Primary human hepatocytes (PHH) were thawed and resuspended in hepatocyte thawing medium (Xenotech, cat #K8600/K8650) followed by centrifugation. The supernatant was discarded, and the pelleted cells were resuspended in hepatocyte plating medium (Xenotech, cat #K8200). Cells were counted via hemocytometer and plated on Bio-coat collagen-I coated 96-well plates at a density of 25,000 cells/well for PHH, 25,000 cells/well for PMH, and 50,000 cells/well for PCH in 100 μl of plating media. Plated cells were allowed to settle and adhere for 6 hours in a tissue culture incubator at 37° C. and 5% CO₂ atmosphere. After incubation cells were checked for monolayer formation after which the plating media was aspirated and replaced with 100 μl of culture media (Xenotech, cat #K8300). Media was replaced every 24 hours for the duration of the experiment.

Primary human hepatocytes were transfected as previously described with LNP formulated oRNAs. Twenty-four hours post-transfection, the media was removed and the cells were trypsinized. The trypsinized cells were neutralized with PBS supplemented with 10% FBS, harvested, and transferred to a tube. The tube was centrifuged to pellet the cells and the supernatant was aspirated. The pellet was stored at −80° C. prior to lysis. For lysis the cells were thawed on ice and were lysed with 100 μL/well RIPA buffer (Boston Bio Products, Cat. BP-115) plus freshly added 1 mM DTT, and 250 U/ml Benzonase (EMD Millipore, cat #71206-3), and protease inhibitor mixture consisting of complete protease inhibitor cocktail (Sigma, cat #11697498001). Cells were kept on ice for 30 minutes at which time NaCl (1M final concentration) was added. Cell lysates were thoroughly mixed and retained on ice for 30 min. The whole cell extracts (WCE) were centrifuged to pellet debris. A Bradford assay (Bio-Rad, cat #500-0001) was used to assess protein content of the lysates. The Bradford assay procedure was completed according to the manufacturer's protocol. Extracts were stored at −20° C. prior to use. Western blots were performed to assess GFP protein levels. Whole cell extract lysates were mixed with Laemmli buffer and denatured at 95° C. for 10 min. Western blots were run using the NuPage system on 4-12% Bis-Tris gels (ThermoFisher, cat #NPO335BOX) according to the manufacturer's protocol followed by wet transfer onto 0.45 m nitrocellulose membrane (ThermoFisher, cat #LC2001). After transfer membranes were rinsed thoroughly with water and stained with Ponceau S solution (Boston Bio Products, cat #ST-180) to confirm complete and even transfer. Blots were blocked using 5% Dry Milk in TBS for 30 minutes on a lab rocker at room temperature. Blots were rinsed with 1× TBST (Boston BioProducts, cat #IBB-180) and probed with mouse dylight 680-tagged anti-GFP monoclonal antibody (ThermoFisher, cat #MA515256D680) at 1:1,000 in 1× TBST. Anti-p-actin or GAPDH was used as a loading control (ThermoFisher, cat #AM4302/AM4300) at 1:4,000 in 1× TBST and incubated simultaneously with the GFP primary antibody. Blots were sealed in a bag and kept overnight at 4° C. on a lab rocker. After incubation, blots were rinsed 3 times for 5 minutes each in 1× TBST and probed with mouse secondary antibodies (ThermoFisher, cat #PI35519) at 1:25,000 each in 1× TBST for 30 minutes at room temperature. After incubation, blots were rinsed 3 times for 5 minutes each in 1× TBST. Blots were visualized and analyzed using a Licor Odyssey system.

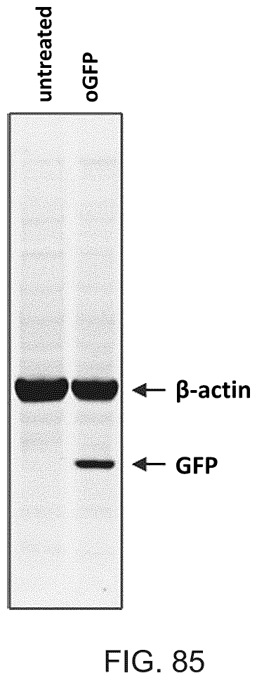

As shown in the western blot in FIG. 85, circular RNAs containing a TIE is capable of successfully encoding a GFP protein in primary human hepatocytes when transfected in vivo with an LNP.

### Example 65

### In Vitro Expression Offirefly Luciferase in Circular RNA Encoding Firefly Luciferase in Mouse Myoblast and Primary Human Skeletal Muscle Myoblast Cells Using Lipofectamine.

A circular RNA construct comprising a TIE and coding element encoding firefly luciferase protein.

Primary human skeletal muscle (HSkM) cells (Lonza, cat #20TL356514) were thawed in a 37° C. water bath and plated at recommended seeding density (3,000 to 5,000 per cm²) in SkGM-2 BulletKit growth media (Lonza, cat #CC-3245) and allowed to grow overnight. Cells were detached using ReagentPack subculture reagents (Lonza, cat #CC-5034) and plated on tissue culture grade 96-well plates at recommended seeding density and allowed to grow overnight in a tissue culture incubator at 37° C. and 5% CO₂ atmosphere or to 70-80% confluency with growth media changed every 2 days.

For one 96-well plate reaction, 0.3 μL of Lipofectamine-3000 transfection reagent (Lipo3K) (ThermoFisher, cat #L3000015) was mixed with 5 μL Opti-MEM reduced serum media (ThermoFisher, cat #51985091). In a separate tube, per reaction, firefly luciferase (f.luc) oRNA (at 10-200 ng) was combined with 5 μL Opti-MEM and 0.2 μL P3000TM enhancer reagent (ThermoFisher, cat #L3000015). Equal volumes of Lipo3K/Opti-MEM mix was combined with oRNA/Opti-MEM mix and incubated at room temperature for 15 min. The Lipo3K/oRNA mixture was added to each well to be transfected and placed in a tissue culture incubator at 37° C. and 5% CO₂ atmosphere for 24 hours.

After 24 hours, the transfection plates were removed from the incubator and allowed to equilibrate to room temperature for 15 minutes. A volume of 100 μL of firefly luciferase one-step glow assay working solution (Pierce, cat #16196) was added to each well. The plate was placed on a microplate shaker (ThermoFisher, cat #572050) and mixed at 300 rpm for 3 minutes. Post-mixing, the plate was allowed to incubate at room temperature for 10 minutes. Luminescence was read using a Varioskan or Bio-Tek Cytation5 instrument.

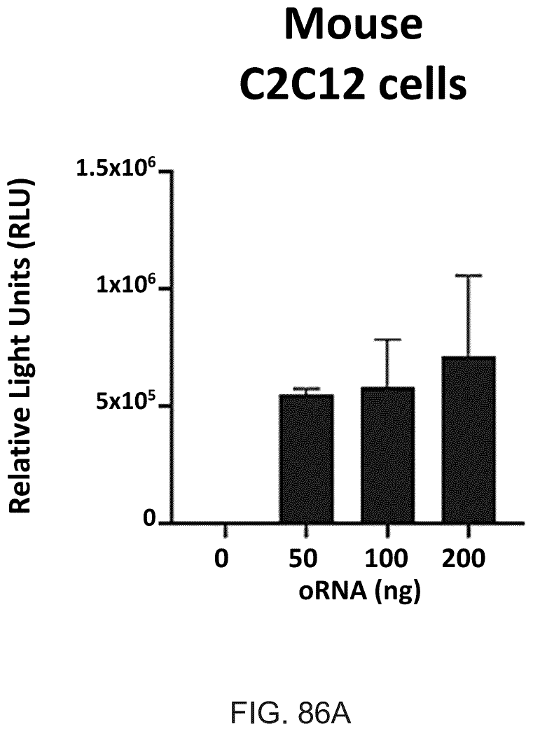

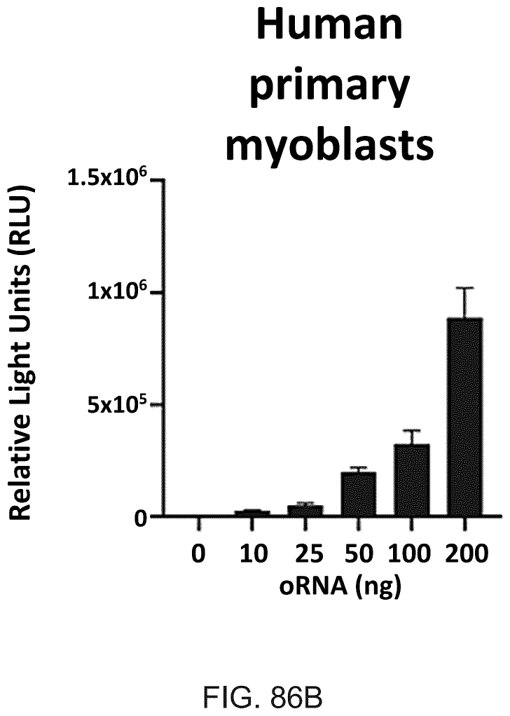

As shown in FIG. 86A and FIG. 86B, circular RNAs comprising a TIE is capable of driving firefly luciferase protein expression in myoblasts from different species in a dose-dependent manner when transfected in vitro with lipofectamine.

### Example 66

In Vitro Expression of Firefly Luciferase in Circular RNA Encoding Firefly Luciferase in Differentiated Primary Human Skeletal Muscles Myotubes

A circular RNA construct comprising a TIE and coding element encoding firefly luciferase protein.

Primary human skeletal muscle (HSkM) cells (Lonza, cat #20TL356514) were thawed in a 37° C. water bath and plated at recommended seeding density (3,000 to 5,000 per cm²) in SkGM-2 BulletKit growth media (Lonza, cat #CC-3245) and allowed to grow overnight. Cells were detached using ReagentPack subculture reagents (Lonza, cat #CC-5034) and plated on tissue culture grade 96-well plates at recommended seeding density and allowed to grow overnight in a tissue culture incubator at 37° C. and 5% CO₂ atmosphere or to 70-80% confluency with growth media changed every 2 days. Once cells reached 70-80% confluency, growth media was removed, cells were washed twice in 1×PBS (Gibco, cat #10010023) and changed to differentiation media consisting of F-10 (1×) (Gibco, cat #11550-043) supplemented with 2% Horse Serum (Gibco, cat #26050088) and 1% Pen-Strep (Gibco, cat #15140-122). Media was changed daily for 5 to 6 days until nearly all myoblasts had fused to form myotubes.

For one 96-well plate reaction, 0.3 μL of Lipofectamine-3000 transfection reagent (Lipo3K) (ThermoFisher, cat #L3000015) was mixed with Sul Opti-MEM reduced serum media (ThermoFisher, cat #51985091). In a separate tube, per reaction, firefly luciferase (f.luc) oRNA (at 10-200 ng) was combined with 5 μL Opti-MEM and 0.2 μL P3000TM enhancer reagent (ThermoFisher, cat #L3000015). Equal volumes of Lipo3K/Opti-MEM mix was combined with oRNA/Opti-MEM mix and incubated at room temperature for 15 min. The Lipo3K/oRNA mixture was added to each well to be transfected and placed in a tissue culture incubator at 37° C. and 5% CO₂ atmosphere for 24 hours.

After 24 hours, transfection plates were removed from the incubator and allowed to equilibrate to room temperature for 15 minutes. A volume of 100 μL of Firefly Luc one-step glow assay working solution (Pierce, cat #16196) was added to each well. The plate was placed on a microplate shaker (ThermoFisher, cat #S72050) and mixed at 300 rpm for 3 minutes. Post-mixing, the plate was allowed to incubate at room temperature for 10 minutes. Luminescence was read using a Varioskan or Bio-Tek Cytation5 instrument.

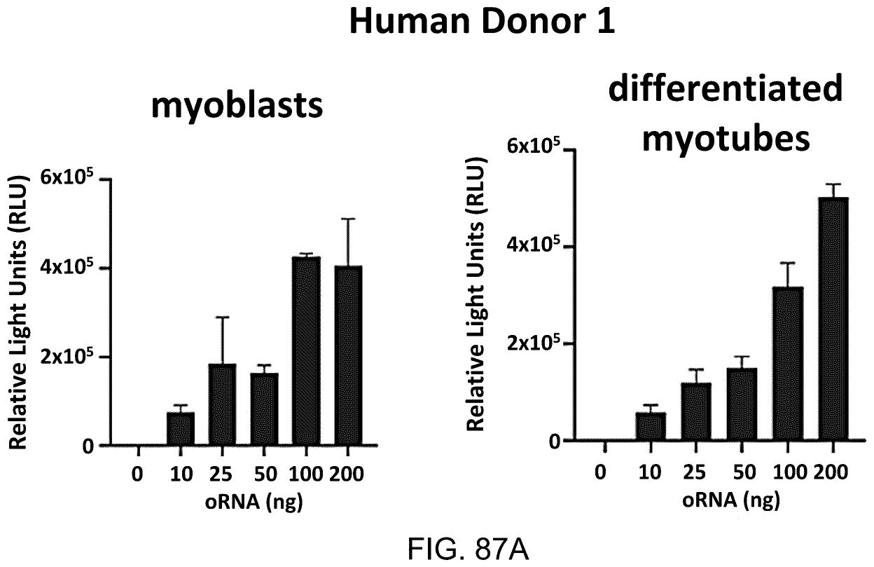

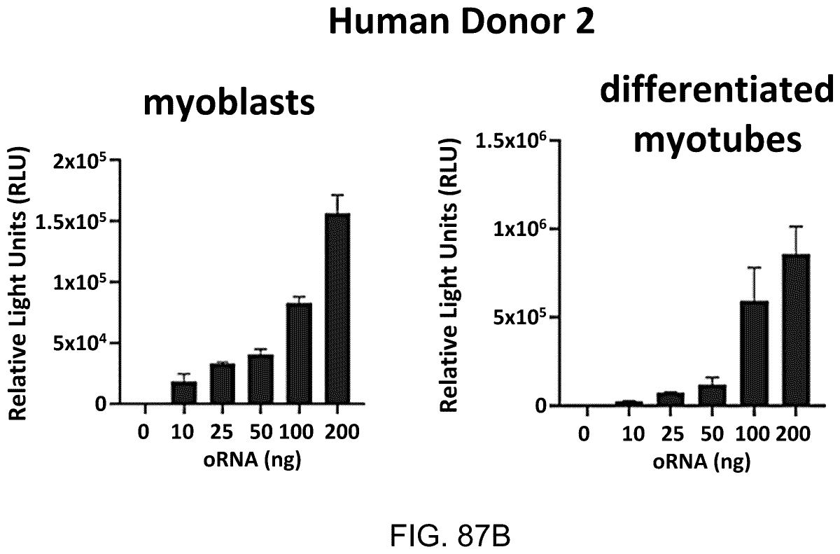

As shown in FIG. 87A and FIG. 87B, a circular RNA comprising a TIE is capable of driving firefly luciferase protein expression in primary muscles cells through differentiated states (e.g., in myoblast and differentiated myotubes) in multiple human donors in a dose-dependent manner when transfected in vitro with lipofectamine.

### Example 67

### Cell-Free In Vitro Translation of Circular RNAs Containing TIEs

A cell-free rabbit reticulocyte in vitro translation assay (Promega, cat #L4540) was completed to characterize protein products from various RNA templates. Both linear mRNA and circular oRNA templates were used in the assay and reaction components were assembled according to the manufacturer's protocol. Prior to assay, RNA templates were denatured at 65° C. for 3 minutes and immediately cooled on ice. All reaction components were assembled on ice. Flexi Rabbit Reticulocyte kit components, complete amino acid mixture (Promega, cat #L5061), RNAsin TIEuclease inhibitor (Promega, cat #N2111), and transcend tRNA (Promega, cat #L5061) were added to denatured RNA templates. The reaction was vortexed to mix and incubated at 30° C. for 60 minutes and then placed on ice.

The reaction mixture was added to 1× sample buffer (ThermoFisher, cat #NP0007) and heated at 70° C. for 15 minutes. The denatured protein sample was loaded onto 4-12% Bis-Tris gels (ThermoFisher, cat #NPO335BOX). Gel electrophoresis was completed, and the gel was wet transferred onto 0.45 m nitrocellulose membrane (ThermoFisher, cat #LC2001). Post-transfer, the membrane was blocked for 1 hour with rocking in freshly made TBS with 0.5% Tween-20 (Boston Bioproducts Inc Cat #IBB-180). The membrane was incubated for 45 min with rocking with streptavidin-AP (Promega, cat #V5591) at a 1:2,500 dilution. The membrane was rinsed with four cycles of: twice with TBST and twice with deionized water for 1 min per rinse. The membrane was incubated with Western Blue substrate (Promega, cat #S3841) for 45 minutes and the membrane was rinsed in water and scanned on a LICOR Odyssey CLx imaging system.

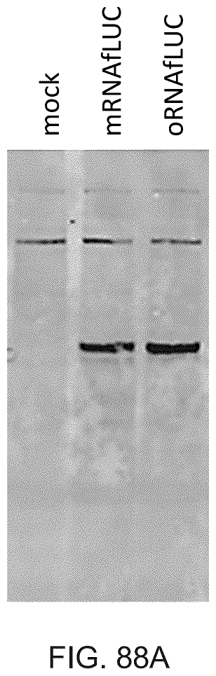

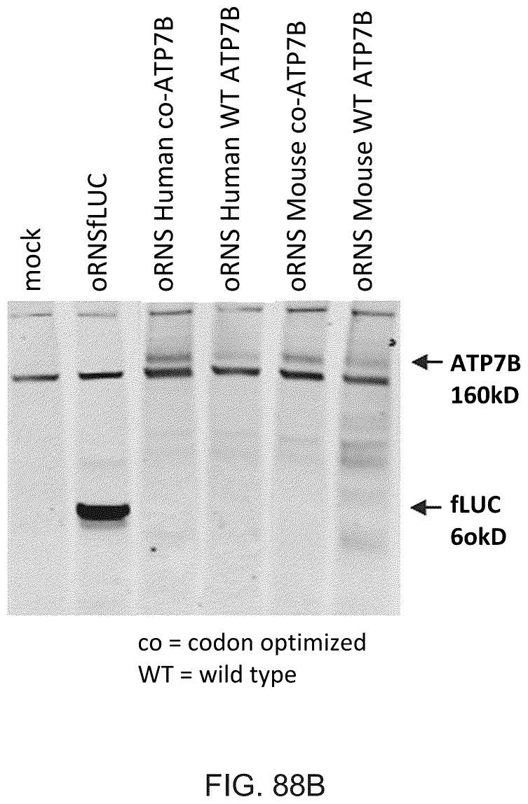

As shown in FIG. 88A and FIG. 88B, a circular RNA comprising a TIE is capable of driving protein expression in a cell-free lysate, independent of any cell type. FIG. 88A illustrates expression of firefly luciferase from a linear or circular RNA input. FIG. 88B illustrates expression of human and mouse ATP7B proteins with different codon optimization (co) approaches compared to wild-type native sequence (WT). The codon optimized-circular RNAs expressing ARP7B protein and the circular RNA expressing firefly luciferase shoed protein full-length protein expression.

### Example 68

### TIE Selection Methodology

Putative TIEs were identified for activity assessment from sequences in GenBank. Briefly, Riboviria and Unclassified Virus sequences greater than 1 kb in length were identified. 5′ and intergenic UTRs were extracted based on putative CDS start and end sites with a minimum length cutoff of 250 nt. Reverse sequences were also collected for negative sense CDS annotations. For genera not expected to contain TIE sequences, a few noncoding regions per genus were selected at random. Duplicates, >10 nt repeat-containing sequences, sequences with both XbaI and BamHI sites, and low-quality sequences (non acgt) were culled, then sequences were clustered through CDHit with an 80% sequence similarity cutoff for clustering; representative sequences from each cluster were selected for further study. For unclassified sequences or sequences expected to contain a TIE, all 5′ and intergenic UTRs were selected; duplicates, >10 nt repeat-containing sequences, sequences with both XbaI and BamHI sites, and low-quality sequences (non acgt) were culled, then sequences were clustered through CDHit with a 95% (low risk, known IRESs) sequence similarity cutoff for clustering; representative sequences from each cluster were selected for further study. For both strategies, sequences shorter than 300 nt or unable to be synthesized due to sequence complexity were eliminated.

### Example 69

### Example 69A: TIE Activity in Primary Human T Cells

Nucleic acid sequences containing putative TIEs were inserted into a circular RNA (oRNA) construct prior to the start codon of a Gaussia luciferase reporter sequence. oRNA containing the TIE was synthesized and purified. Purified oRNA was formulated into lipid nanoparticles. LNP-oRNA was transfected into T cells in vitro. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours indicates higher oRNA stability due to TIE function.

### Example 69B: TIE Activity in Primary Human Hepatocytes

Nucleic acid sequences containing putative TIEs were inserted into a circular RNA (oRNA) construct prior to the start codon of a Gaussia luciferase reporter sequence. oRNA containing the TIE was synthesized and purified. Purified oRNA was formulated into lipid nanoparticles. LNP-oRNA was transfected into hepatocytes in vitro. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours indicates higher oRNA stability due to TIE function.

### Example 69C: TIE Activity in Primary Human Myotubes

Nucleic acid sequences containing putative TIEs were inserted into a circular RNA (oRNA) construct prior to the start codon of a Gaussia luciferase reporter sequence. oRNA containing the TIE was synthesized and purified. Purified oRNA was formulated into lipid nanoparticles. LNP-oRNA was transfected into human myotubes in vitro. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours indicates higher oRNA stability due to TIE function.

### Example 70

### TIE Tissue Tropism

Select TIE-containing oRNAs were formulated into LNPs. LNP-oRNAs were transfected into T cells, hepatocytes, and myotubes. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours may indicate higher oRNA stability due to TIE function. TIE activity was compared between cell types and differences resulting from TIE tissue preference were noted. Differences may be a result of the TIE engaging proteins that show tissue-specific expression and promoting enhanced translation initiation, degradation, or stability.

### Example 71

### Example 71A: TIE Deletion Scanning

Select TIE sequences with progressive deletions from either the 5′ end or 3′ end of the TIE were inserted into a circular RNA (oRNA) construct prior to the start codon of a Gaussia luciferase reporter sequence. oRNA containing the TIE variant was synthesized and purified. Purified oRNA was formulated into lipid nanoparticles. LNP-oRNA was transfected into human primary T cells. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours may indicate higher oRNA stability due to TIE function. Expression or stability impairment due to progressive deletions identifies the core functional unit of the TIE.

### Example 71B: TIE Variant Generation and Identification

Select TIE-containing oRNA synthesis plasmids were subjected to error-prone PCR to introduce random mutations into the PCR product. PCR product was used as a template for oRNA synthesis. Purified oRNA was formulated into LNPs and transfected into primary human T cells. Polysome fractions were harvested from T cells at 6, 24, 48, and 72 hours post-transfection by HPLC. RNA associated with each polysome fraction was extracted from polysome fractions and sequenced by NGS. TIE mutation enrichment in each polysome fraction at each time point was analyzed to identify mutations that 1) maintain or improve translation activity from the TIE and/or 2) improve stability of the oRNA.

### Example 71C: TIE Single and Multi-Variant Validation

Nucleic acid sequences containing putative beneficial TIE mutations from example 6 alone or in combination were inserted into a circular RNA (oRNA) construct prior to the start codon of a Gaussia luciferase reporter sequence. oRNA containing the TIE variant was synthesized and purified. Purified oRNA was formulated into lipid nanoparticles. LNP-oRNA was transfected into human primary T cells. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours indicates higher oRNA stability due to TIE function.

### Example 72

### Example 72A: Selection of Eukaryotic TIEs

Selection of eukaryotic TIEs. Putative eukaryotic TIEs were identified using several databases. TIEs selected include sequences 40-1578 nucleotides in length and may or may not contain identified modification (m6A) sites.

### Example 72B: TIEs Containing Modified Nucleotides (m6A)

Nucleic acid sequences containing putative TIEs were inserted into a circular RNA (oRNA) construct preceding the coding region of a Gaussia luciferase reporter sequence. oRNAs were synthesized with a titration of modified nucleotide. Purified oRNA was formulated into lipid nanoparticles. LNP-oRNAs were transfected into T cells, hepatocytes, and myotubes. Supernatant was harvested and replaced 24 and 48 hours after transfection, and Gaussia luciferase expression from oRNA was determined using a coelenterazine-containing detection reagent and luminometer. Higher luminescence of modified nucleotide containing TIEs at 24 hours indicates necessity for modification for enhanced function. Higher luminescence at 48 hours relative to 24 hours indicates modified nucleotide containing TIEs enhance stability of oRNA.

### Example 73

Expression of TIEs In Cells Undergoing Oxidative and/or Hypoxic Stress

Nucleic acid sequences containing putative TIEs were inserted into a circular RNA (oRNA) construct preceding the coding region of a Gaussia luciferase reporter sequence. Purified oRNA was formulated into lipid nanoparticles. Hepatocytes were treated with hydrogen peroxide to induce oxidative stress or CoCl₂ to induce hypoxic stress. LNP-oRNA was transfected into hepatocytes (under hypoxic stress, oxidative stress, or untreated) in vitro. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours may indicate higher oRNA stability due to TIE function.

### Example 74

### Aptamer as a TIE

Nucleic acid sequences containing aptamers against translation initiation factors (ie eIF4E, eIF4G, eIF4a) were inserted into a circular RNA (oRNA) construct preceding the coding region of a Gaussia luciferase reporter sequence. Purified oRNA was formulated into lipid nanoparticles. LNP-oRNAs were transfected into hepatocytes. Supernatant was harvested and replaced 24 and 48 hours after transfection, and Gaussia luciferase expression from oRNA was determined using a coelenterazine-containing detection reagent and luminometer. Higher luminescence at 24 hours indicates higher TIE function. Higher luminescence at 48 hours relative to 24 hours may indicate higher oRNA stability due to TIE function.

### Example 75

### Tandem TIEs

Select combinations of viral, eukaryotic, and/or aptamer TIEs were inserted into a circular RNA (oRNA) construct preceding the coding region of a Gaussia luciferase reporter sequence. oRNAs were synthesized with a titration of modified nucleotide. Purified oRNA was formulated into lipid nanoparticles. LNP-oRNAs were transfected into T cells, hepatocytes, and myotubes. Supernatant was harvested and replaced 24 and 48 hours after transfection, and Gaussia luciferase expression from oRNA was determined using a coelenterazine-containing detection reagent and luminometer. Higher luminescence of constructs containing multiple TIEs at 24 hours indicates a synergy of TIEs. Higher luminescence at 48 hours relative to 24 hours may indicate having multiple TIEs in one construct enhance stability of oRNA.

### Example 76

### Example 76A: Coding Aptamers to Enhance Cap-Independent Translation

Certain aptamers that bind to eIF4E, eIF4a, and other translation initiators are known to inhibit translation by forcing the proteins to adopt a non-functional conformation. Nucleic acid sequences containing aptamers against translation initiation factors (ie eIF4E, eIF4a) were inserted into a circular RNA (oRNA) construct preceding a functional TIE and the coding region of a Gaussia luciferase reporter sequence. Purified oRNA was formulated into lipid nanoparticles. LNP-oRNAs were transfected into hepatocytes. Supernatant was harvested and replaced 24 and 48 hours after transfection, and Gaussia luciferase expression from oRNA was determined using a coelenterazine-containing detection reagent and luminometer. Higher luminescence at 24 hours indicates a preference for cap-independent translation. Higher luminescence at 48 hours relative to 24 hours indicates higher oRNA stability due to inhibition of cap-dependent translation.

### Example 76B: Coding Aptamers to Enhance Cap-Independent Translation

Co-transfection of oRNA and aptamers. Certain aptamers that bind to eIF4E, eIF4a, and other translation initiators are known to inhibit translation by forcing the proteins to adopt a non-functional conformation. Nucleic acid sequences containing aptamers against translation initiation factors (i.e., eIF4E, eIF4a) were co-transfected with a circular RNA (oRNA) containing a TIE and the coding region of a Gaussia luciferase reporter. Purified aptamer and oRNA were formulated into lipid nanoparticles together. LNP-oRNAs were transfected into hepatocytes. Supernatant was harvested and replaced 24 and 48 hours after transfection, and Gaussia luciferase expression from oRNA was determined using a coelenterazine-containing detection reagent and luminometer. Higher luminescence at 24 hours indicates a preference for cap-independent translation. Higher luminescence at 48 hours relative to 24 hours may indicate higher oRNA stability due to inhibition of cap-dependent translation.

### Example 77

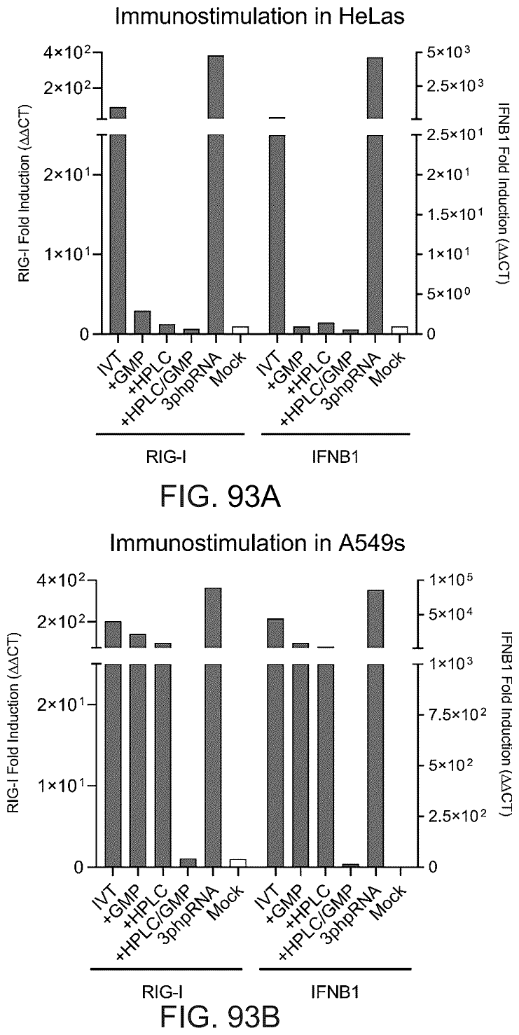

Immunogenicity of circular RNAs was assessed. Induction of RIG-1 and IFNB1 RNA expressions, markers of immune stimulation, was measured using RT-cPCR following transfection of the cells with various RNA preparations. All RNA preparations except for the commercially available 3phpRNA were produced using in vitro transcription (IVT) and circularization of RNA comprising an Anabaena permuted intron, GLuc reading frame, strong homology arms, 5′ and 3′ spacers, and a CVB3 IRES. In both HeLa cells (FIG. 93A) and A594 cells (FIG. 93B), the highest levels of RIG-1 and IFNB1 were induced by the IVT reaction product prepared in the absence of excess GMP and by the positive control 3phpRNA.

### Example 78—IRES Selection and Screening

Approximately 3,000 internal ribosome entry site (IRES) sequence candidates were computationally identified from untranslated regions (UTRs), e.g., viral UTRs, in partial and complete viral sequence available in the art (e.g., Genbank). Each DNA template was cloned to include a IRES sequence selected from SEQ ID NOs: 1-2983 and 3282-3287 or a fragment thereof, optionally in combination with barcode sequences selected from [3289-6271 and 6570-6575]. Exemplary constructs included a 5′ enhanced intron element, a 5′ enhanced exon element, an TRES candidate, a coding element encoding Gaussia Luciferase, a 3′ enhanced exon element, and a 3′ enhanced intron element. Engineered circular RNA (oRNA) was generated from the DNA template by in vitro transcription (IVT); and oRNA was subsequently purified from non-circular RNA components of the IVT reaction.

Purified oRNA was nanoprecipitated with lipids to form LNP-oRNA constructs. LNPs were formulated first by creating empty lipid nanoparticles using a Precision Nanosystems Ignite instrument with a “NextGen” mixing chamber. Ethanol phase contained Lipid 39 from Table 2, DSPC, cholesterol, and DMG-PEG 2000 (Avanti Polar Lipids, Inc., Alabaster, AL) at a molar ratio of 45:9:44:2 was combined with 50 mM sodium acetate buffer at pH 4.5. A 3:1 aqueous to ethanol mixing ratio was used. The empty LNPs were then dialyzed in 1 L of 1×PBS and exchanged 2 times over 18 hours. Dialyzed empty LNPs were then mixed with a final circular RNA concentration of 40 μg/mL in 50 mM sodium acetate buffer at pH 4.5. A 3:1 aqueous to empty lipid nanoparticle mixing ratio was used. The LNPs final ionizable lipid to RNA phosphate ratio was at 6.0.

Primary human skeletal myotubes, primary human hepatocytes, and activated primary human T cells were treated with formulated LNP-oRNA in triplicate. Protein expression in supernatant was assessed by luminescence measurements 48 hours following transfection.

Total luminescent signal was normalized across replicate plates to control for plate-wide differences between replicates due to flash assay readout timing. Plates with positive controls having <1.5× signal over background were excluded from analysis. Background was subtracted based on empty wells. Each test well replicate was normalized to the average of the plate controls.

Table 4 provides the protein expression and stability measurements for the screening. Measured numbers were normalized to a control (caprine kobuvirus IRES, SEQ ID NO: 3282) and represented as a fold change.

TABLE 4
I. Expression and functional stability screening of IRES

| | [Mod] | | [Mod] |
|---|---|---|---|
| SEQ | Fold | [Mod] T | PHH |
| ID | Change | Fold | Fold |
| NO | Myo | Change | Change |
| 97 | 0.260167 | 0.353177 | 1.163467 |
| 98 | 0.290648 | 0.677026 | 0.78802 |
| 99 | 0.163908 | 0.27666 | 1.020326 |
| 100 | 0.495163 | 0.48261 | 1.22777 |
| 101 | 0.533025 | 0.852995 | 1.370347 |
| 102 | 0.044695 | 0.030995 | 0.514093 |
| 103 | 0.045577 | 0.039508 | 0.647085 |
| 104 | 0.076071 | 0.168841 | 0.774536 |
| 105 | 0.249558 | 0.329006 | 0.642043 |
| 106 | 0.352419 | 0.648041 | 1.09325 |
| 107 | 0.287896 | 0.937122 | 1.119079 |
| 109 | 0.249157 | 0.626121 | 0.853308 |
| 110 | 0.242425 | 0.513277 | 0.717483 |
| 111 | 0.222321 | 0.465065 | 0.786969 |
| 112 | 0.371086 | 0.636962 | 0.807476 |
| 113 | 0.317071 | 0.43731 | 0.827882 |
| 114 | 0.05835 | 0.068124 | 0.789812 |
| 115 | 0.050735 | 0.046932 | 0.425032 |
| 116 | 0.039211 | 0.030739 | 0.529925 |
| 117 | 0.042734 | 0.046123 | 0.425775 |
| 118 | 0.154586 | 0.456184 | 0.666903 |
| 119 | 0.198021 | 0.987593 | 0.62119 |
| 120 | 0.275525 | 0.701885 | 0.489288 |
| 121 | 0.627572 | 1.028933 | 0.997696 |
| 122 | 0.388602 | 0.8876 | 0.832163 |
| 123 | 0.039714 | 0.030111 | 0.638047 |
| 125 | 0.382696 | 0.641754 | 0.649185 |
| 126 | 0.057929 | 0.06647 | 0.645602 |
| 127 | 0.293568 | 0.685807 | 0.674465 |
| 128 | 0.489317 | 0.583675 | 1.021214 |
| 129 | 0.053695 | 0.089454 | 0.668895 |
| 130 | 0.040577 | 0.060757 | 0.572842 |
| 131 | 0.039511 | 0.033359 | 0.53476 |
| 132 | 0.039977 | 0.031182 | 0.564324 |
| 133 | 0.039326 | 0.030041 | 0.541046 |
| 134 | 0.046625 | 0.052943 | 0.46727 |
| 136 | 0.047864 | 0.042316 | 0.428555 |
| 138 | 0.372287 | 0.637832 | 0.615651 |
| 139 | 0.059696 | 0.097316 | 0.520599 |
| 140 | 0.16008 | 0.399455 | 0.715372 |
| 141 | 0.040253 | 0.031062 | 0.546956 |
| 142 | 0.038521 | 0.027425 | 0.46798 |
| 143 | 0.03901 | 0.032654 | 0.420921 |
| 145 | 0.03927 | 0.026297 | 0.635934 |
| 146 | 0.043229 | 0.034645 | 0.587329 |
| 147 | 0.042637 | 0.036624 | 0.559359 |
| 148 | 0.039126 | 0.029094 | 0.736827 |
| 149 | 0.039186 | 0.028926 | 0.711791 |
| 150 | 0.03864 | 0.027048 | 0.514678 |
| 151 | 0.037309 | 0.026948 | 0.493258 |
| 152 | 0.038449 | 0.026258 | 0.615534 |
| 153 | 0.038341 | 0.025876 | 0.695107 |
| 154 | 0.0402 | 0.0304 | 0.722679 |
| 155 | 0.038723 | 0.027351 | 0.586864 |
| 156 | 0.038511 | 0.027142 | 0.627475 |
| 157 | 0.039049 | 0.027127 | 0.731503 |
| 158 | 0.038048 | 0.027701 | 0.672155 |
| 159 | 0.038668 | 0.025816 | 0.61191 |
| 160 | 0.039335 | 0.026247 | 0.5674 |
| 161 | 0.038824 | 0.025876 | 0.492452 |
| 162 | 0.038216 | 0.023393 | 0.575165 |
| 163 | 0.038264 | 0.027191 | 0.52053 |
| 164 | 0.038197 | 0.026151 | 0.524974 |
| 165 | 0.037817 | 0.026532 | 0.561699 |
| 166 | 0.038844 | 0.026828 | 0.575901 |
| 167 | 0.038524 | 0.025708 | 0.69976 |
| 168 | 0.038788 | 0.025543 | 0.731934 |
| 169 | 0.039231 | 0.027338 | 0.432891 |
| 170 | 0.037986 | 0.022969 | 0.541642 |
| 171 | 0.038154 | 0.027212 | 0.582587 |
| 172 | 0.037112 | 0.026558 | 0.639476 |
| 173 | 0.037335 | 0.026078 | 0.504809 |
| 174 | 0.037049 | 0.025905 | 0.577144 |
| 175 | 0.037822 | 0.025498 | 0.530502 |
| 177 | 0.038266 | 0.026051 | 0.561219 |
| 178 | 0.039045 | 0.027092 | 0.500802 |
| 179 | 0.037153 | 0.026609 | 0.50571 |
| 180 | 0.038755 | 0.028951 | 0.487148 |
| 181 | 0.037539 | 0.025831 | 0.659122 |
| 182 | 0.03747 | 0.025334 | 0.496612 |
| 183 | 0.038763 | 0.025478 | 0.768879 |
| 184 | 0.043604 | 0.030907 | 0.689648 |
| 185 | 0.039562 | 0.027297 | 0.687583 |
| 186 | 0.039007 | 0.026413 | 0.665244 |
| 187 | 0.038049 | 0.026641 | 0.587503 |
| 188 | 0.037709 | 0.026875 | 0.671188 |
| 189 | 0.037762 | 0.027089 | 0.52406 |
| 190 | 0.03731 | 0.026211 | 0.418576 |
| 191 | 0.038235 | 0.02599 | 0.484504 |
| 192 | 0.039939 | 0.027233 | 0.483419 |
| 194 | 0.020192 | 0.00648 | 0.519074 |
| 195 | 0.019531 | 0.006334 | 0.376436 |
| 196 | 0.019542 | 0.00641 | 0.498646 |
| 197 | 0.019244 | 0.006324 | 0.402627 |
| 198 | 0.018573 | 0.006291 | 0.419065 |
| 199 | 0.019198 | 0.006785 | 0.34161 |
| 200 | 0.019364 | 0.00696 | 0.385662 |
| 201 | 0.020124 | 0.006802 | 0.336954 |
| 202 | 0.019957 | 0.006958 | 0.433697 |
| 203 | 0.019453 | 0.006298 | 0.462718 |
| 204 | 0.019494 | 0.006471 | 0.441202 |
| 205 | 0.019442 | 0.006373 | 0.629439 |
| 206 | 0.018867 | 0.006885 | 0.374898 |
| 207 | 0.01914 | 0.006134 | 0.460931 |
| 208 | 0.018981 | 0.006092 | 0.433478 |
| 209 | 0.019624 | 0.006283 | 0.410556 |
| 210 | 0.019591 | 0.007049 | 0.588881 |
| 211 | 0.019397 | 0.006595 | 0.542165 |
| 212 | 0.019568 | 0.00626 | 0.532913 |
| 213 | 0.018989 | 0.006333 | 0.547859 |
| 214 | 0.01915 | 0.006268 | 0.425401 |
| 216 | 0.019187 | 0.006268 | 0.524257 |
| 217 | 0.019397 | 0.006238 | 0.390937 |
| 218 | 0.019339 | 0.006206 | 0.494667 |
| 219 | 0.018624 | 0.00708 | 0.490922 |
| 220 | 0.019181 | 0.006335 | 0.474431 |
| 221 | 0.019256 | 0.006244 | 0.457017 |
| 222 | 0.019112 | 0.006315 | 0.449704 |
| 223 | 0.01928 | 0.006732 | 0.413984 |
| 224 | 0.019162 | 0.006106 | 0.445024 |
| 225 | 0.019165 | 0.006185 | 0.408971 |
| 226 | 0.018975 | 0.006285 | 0.510492 |
| 227 | 0.019522 | 0.007133 | 0.437234 |
| 228 | 0.019859 | 0.006494 | 0.570592 |
| 229 | 0.019454 | 0.006315 | 0.473748 |
| 230 | 0.018871 | 0.006278 | 0.51804 |
| 231 | 0.019655 | 0.006844 | 0.511603 |
| 232 | 0.019285 | 0.006225 | 0.565024 |
| 233 | 0.019316 | 0.006211 | 0.442953 |
| 235 | 0.019002 | 0.007102 | 0.395003 |
| 236 | 0.018841 | 0.006315 | 0.509341 |
| 237 | 0.019316 | 0.006327 | 0.318761 |
| 238 | 0.019395 | 0.007836 | 0.596039 |
| 239 | 0.019249 | 0.006356 | 0.413194 |
| 241 | 0.019155 | 0.006661 | 0.275657 |
| 242 | 0.019203 | 0.006108 | 0.443553 |
| 243 | 0.019402 | 0.006124 | 0.451916 |
| 244 | 0.018854 | 0.007129 | 0.467265 |
| 245 | 0.019175 | 0.006401 | 0.543468 |
| 246 | 0.019056 | 0.007055 | 0.4824 |
| 247 | 0.018824 | 0.006353 | 0.484173 |
| 248 | 0.019655 | 0.006175 | 0.368398 |
| 249 | 0.01946 | 0.006185 | 0.396805 |
| 250 | 0.019 | 0.006117 | 0.480145 |
| 252 | 0.01904 | 0.007513 | 0.39182 |
| 253 | 0.018899 | 0.006977 | 0.575079 |
| 254 | 0.019338 | 0.006347 | 0.526008 |
| 255 | 0.019193 | 0.006255 | 0.287479 |
| 256 | 0.019022 | 0.006292 | 0.433444 |
| 257 | 0.01915 | 0.006647 | 0.428502 |
| 258 | 0.019567 | 0.006018 | 0.468423 |
| 259 | 0.019534 | 0.006067 | 0.58953 |
| 260 | 0.019436 | 0.007053 | 0.357243 |
| 261 | 0.019714 | 0.006449 | 0.480901 |
| 262 | 0.018951 | 0.006418 | 0.40858 |
| 263 | 0.019192 | 0.006212 | 0.560046 |
| 264 | 0.019612 | 0.006356 | 0.552301 |
| 265 | 0.019321 | 0.006195 | 0.401567 |
| 266 | 0.019369 | 0.006648 | 0.604516 |
| 267 | 0.019151 | 0.006684 | 0.464703 |
| 268 | 0.018286 | 0.007032 | 0.4753 |
| 269 | 0.01937 | 0.006374 | 0.5176 |
| 270 | 0.019261 | 0.006259 | 0.499406 |
| 271 | 0.019395 | 0.00619 | 0.412264 |
| 272 | 0.018911 | 0.006199 | 0.421016 |
| 274 | 0.019303 | 0.006349 | 0.455375 |
| 276 | 0.019357 | 0.006325 | 0.376791 |
| 278 | 0.019529 | 0.006257 | 0.583499 |
| 279 | 0.019217 | 0.006392 | 0.4111 |
| 281 | 0.019185 | 0.006136 | 0.587463 |
| 282 | 0.019681 | 0.006236 | 0.688522 |
| 283 | 0.020003 | 0.006456 | 0.361814 |
| 284 | 0.021001 | 0.008113 | 0.463016 |
| 285 | 0.019166 | 0.006828 | 0.350735 |
| 286 | 0.019521 | 0.006951 | 0.480012 |
| 287 | 0.019513 | 0.006754 | 0.469042 |
| 288 | 0.018469 | 0.006294 | 0.484396 |
| 289 | 0.019434 | 0.006129 | 0.551443 |
| 290 | 0.019277 | 0.006906 | 0.508385 |
| 291 | 0.021833 | 0.009489 | 0.384556 |
| 293 | 0.01252 | 0.006427 | 0.356902 |
| 294 | 0.011884 | 0.006276 | 0.322886 |
| 295 | 0.012392 | 0.00627 | 0.447166 |
| 297 | 0.012283 | 0.00621 | 0.567064 |
| 298 | 0.012177 | 0.006287 | 0.464133 |
| 299 | 0.012284 | 0.006151 | 0.348391 |
| 300 | 0.012339 | 0.006071 | 0.502548 |
| 301 | 0.012495 | 0.007116 | 0.35649 |
| 302 | 0.01222 | 0.006362 | 0.411845 |
| 303 | 0.012195 | 0.006278 | 0.26423 |
| 304 | 0.012215 | 0.006262 | 0.552091 |
| 305 | 0.01249 | 0.006788 | 0.42767 |
| 306 | 0.012402 | 0.006165 | 0.371097 |
| 307 | 0.012281 | 0.006139 | 0.520864 |
| 308 | 0.012497 | 0.00617 | 0.397038 |
| 309 | 0.012793 | 0.006274 | 0.245538 |
| 310 | 0.012404 | 0.006334 | 0.348876 |
| 311 | 0.012122 | 0.006441 | 0.294773 |
| 312 | 0.012249 | 0.006229 | 0.325353 |
| 313 | 0.012563 | 0.006174 | 0.365834 |
| 314 | 0.012287 | 0.006596 | 0.462958 |
| 315 | 0.012275 | 0.006142 | 0.298952 |
| 316 | 0.012716 | 0.007565 | 0.34122 |
| 317 | 0.012291 | 0.006397 | 0.309566 |
| 318 | 0.012115 | 0.006393 | 0.329808 |
| 319 | 0.01227 | 0.008317 | 0.304387 |
| 320 | 0.012221 | 0.006647 | 0.328903 |
| 321 | 0.012186 | 0.007163 | 0.399922 |
| 322 | 0.015035 | 0.031123 | 0.376798 |
| 323 | 0.012308 | 0.006096 | 0.237291 |
| 324 | 0.01192 | 0.006316 | 0.405807 |
| 325 | 0.012466 | 0.00625 | 0.493302 |
| 328 | 0.012498 | 0.00643 | 0.514255 |
| 329 | 0.012432 | 0.006216 | 0.374611 |
| 330 | 0.012053 | 0.006128 | 0.420442 |
| 331 | 0.012416 | 0.006082 | 0.375976 |
| 332 | 0.01236 | 0.006301 | 0.525602 |
| 333 | 0.012418 | 0.006263 | 0.410027 |
| 334 | 0.011605 | 0.006186 | 0.411457 |
| 335 | 0.011728 | 0.006232 | 0.357123 |
| 336 | 0.012106 | 0.006323 | 0.380811 |
| 337 | 0.012118 | 0.006278 | 0.325981 |
| 338 | 0.012218 | 0.006206 | 0.377091 |
| 339 | 0.012209 | 0.006258 | 0.32511 |
| 340 | 0.012372 | 0.006041 | 0.380202 |
| 341 | 0.012382 | 0.00629 | 0.480572 |
| 342 | 0.011896 | 0.006308 | 0.348783 |
| 343 | 0.01204 | 0.006332 | 0.37449 |
| 344 | 0.011928 | 0.006417 | 0.471347 |
| 345 | 0.01169 | 0.006267 | 0.36332 |
| 346 | 0.012345 | 0.006259 | 0.366717 |
| 347 | 0.012182 | 0.006163 | 0.363674 |
| 348 | 0.012163 | 0.00616 | 0.444482 |
| 349 | 0.012659 | 0.006222 | 0.317343 |
| 350 | 0.01182 | 0.006202 | 0.590775 |
| 351 | 0.011972 | 0.006135 | 0.408389 |
| 352 | 0.011885 | 0.006384 | 0.34904 |
| 353 | 0.011969 | 0.006398 | 0.336116 |
| 354 | 0.012428 | 0.006564 | 0.404646 |
| 355 | 0.012092 | 0.006308 | 0.299465 |
| 356 | 0.012409 | 0.006126 | 0.356694 |
| 357 | 0.012519 | 0.006278 | 0.390957 |
| 358 | 0.011963 | 0.006349 | 0.303741 |
| 359 | 0.012077 | 0.006261 | 0.334388 |
| 360 | 0.012007 | 0.006365 | 0.350437 |
| 361 | 0.012282 | 0.007154 | 0.317387 |
| 362 | 0.012469 | 0.007445 | 0.472965 |
| 363 | 0.012436 | 0.006211 | 0.393245 |
| 364 | 0.012106 | 0.006298 | 0.499986 |
| 365 | 0.012805 | 0.009062 | 0.426256 |
| 366 | 0.01212 | 0.006302 | 0.373071 |
| 367 | 0.01206 | 0.006217 | 0.396929 |
| 368 | 0.011994 | 0.006227 | 0.438247 |
| 369 | 0.011901 | 0.006318 | 0.340287 |
| 370 | 0.012341 | 0.007446 | 0.356303 |
| 371 | 0.012601 | 0.006237 | 0.447917 |
| 372 | 0.012511 | 0.007461 | 0.304352 |
| 373 | 0.012419 | 0.006273 | 0.445464 |
| 374 | 0.012212 | 0.006298 | 0.325297 |
| 375 | 0.012262 | 0.006342 | 0.253124 |
| 376 | 0.01206 | 0.00625 | 0.506673 |
| 377 | 0.012314 | 0.006239 | 0.391603 |
| 378 | 0.011869 | 0.006468 | 0.357757 |
| 380 | 0.013014 | 0.007118 | 0.33064 |
| 381 | 0.014152 | 0.007984 | 0.444288 |
| 382 | 0.012422 | 0.006289 | 0.366077 |
| 383 | 0.012649 | 0.0065 | 0.360518 |
| 384 | 0.012302 | 0.006376 | 0.350175 |
| 385 | 0.012345 | 0.006215 | 0.391858 |
| 386 | 0.012388 | 0.006074 | 0.51586 |
| 387 | 0.012519 | 0.007024 | 0.397167 |
| 388 | 0.013891 | 0.00748 | 0.425872 |
| 391 | 0.00426 | 0.025652 | 1.07762 |
| 392 | 0.004221 | 0.024819 | 0.967405 |
| 393 | 0.004402 | 0.026185 | 1.490273 |
| 395 | 0.004363 | 0.024306 | 1.116184 |
| 396 | 0.004078 | 0.025204 | 1.218613 |
| 397 | 0.004141 | 0.025195 | 1.186671 |
| 399 | 0.004058 | 0.025739 | 1.164329 |
| 400 | 0.004405 | 0.026216 | 0.849372 |
| 401 | 0.004166 | 0.025629 | 1.02162 |
| 402 | 0.004116 | 0.025377 | 1.037716 |
| 403 | 0.004734 | 0.028736 | 1.197277 |
| 404 | 0.004247 | 0.025532 | 0.859999 |
| 405 | 0.004301 | 0.025699 | 0.866291 |
| 406 | 0.004319 | 0.025744 | 1.453868 |
| 407 | 0.00439 | 0.026383 | 0.93497 |
| 408 | 0.00535 | 0.032252 | 0.865951 |
| 409 | 0.004149 | 0.025932 | 0.961605 |
| 410 | 0.004251 | 0.025634 | 1.117515 |
| 411 | 0.004339 | 0.025073 | 1.08764 |
| 412 | 0.004524 | 0.025372 | 0.873893 |
| 413 | 0.004216 | 0.025102 | 1.007087 |
| 414 | 0.004159 | 0.025802 | 0.964101 |
| 415 | 0.004389 | 0.025762 | 0.882981 |
| 416 | 0.004232 | 0.024642 | 1.018017 |
| 417 | 0.004211 | 0.025603 | 0.850283 |
| 418 | 0.004341 | 0.025106 | 0.846304 |
| 419 | 0.004202 | 0.024844 | 1.080883 |
| 420 | 0.004135 | 0.024946 | 0.97078 |
| 422 | 0.004459 | 0.025873 | 1.058666 |
| 423 | 0.004274 | 0.025787 | 1.226844 |
| 424 | 0.004383 | 0.024211 | 0.850711 |
| 425 | 0.004319 | 0.025464 | 0.973357 |
| 426 | 0.004226 | 0.025422 | 0.838836 |
| 427 | 0.004226 | 0.025762 | 0.973639 |
| 428 | 0.004161 | 0.025186 | 0.86052 |
| 429 | 0.004196 | 0.02512 | 1.028343 |
| 430 | 0.004215 | 0.0256 | 1.062937 |
| 431 | 0.00421 | 0.025733 | 1.00445 |
| 433 | 0.004741 | 0.027408 | 0.950117 |
| 434 | 0.004318 | 0.025754 | 1.092758 |
| 435 | 0.004428 | 0.025757 | 1.018609 |
| 436 | 0.004163 | 0.02512 | 0.727576 |
| 437 | 0.004243 | 0.025229 | 0.780294 |
| 438 | 0.00418 | 0.026218 | 1.039505 |
| 439 | 0.004887 | 0.026537 | 1.117939 |
| 440 | 0.005189 | 0.028072 | 0.889784 |
| 441 | 0.004272 | 0.025361 | 0.836717 |
| 442 | 0.004524 | 0.02738 | 0.921425 |
| 443 | 0.004218 | 0.025148 | 1.162762 |
| 444 | 0.004237 | 0.024847 | 0.728786 |
| 445 | 0.004293 | 0.025134 | 1.175772 |
| 446 | 0.005773 | 0.02595 | 0.999823 |
| 447 | 0.004274 | 0.025853 | 1.006288 |
| 448 | 0.004344 | 0.02546 | 0.863958 |
| 449 | 0.004346 | 0.025705 | 0.920647 |
| 450 | 0.004326 | 0.025476 | 0.816123 |
| 451 | 0.004237 | 0.025069 | 0.846299 |
| 452 | 0.005257 | 0.025596 | 0.800487 |
| 453 | 0.007428 | 0.026513 | 1.037012 |
| 454 | 0.004303 | 0.024553 | 0.94302 |
| 455 | 0.010434 | 0.037939 | 0.690676 |
| 456 | 0.007299 | 0.027948 | 1.099247 |
| 457 | 0.004251 | 0.025526 | 0.872239 |
| 458 | 0.004302 | 0.025162 | 1.050254 |
| 459 | 0.004217 | 0.025242 | 0.856808 |
| 460 | 0.004174 | 0.02491 | 0.771856 |
| 461 | 0.004283 | 0.023773 | 0.825394 |
| 462 | 0.004232 | 0.026042 | 1.06574 |
| 463 | 0.00424 | 0.02602 | 0.834992 |
| 464 | 0.004112 | 0.026109 | 1.215674 |
| 465 | 0.004229 | 0.025226 | 0.796978 |
| 466 | 0.004197 | 0.024889 | 1.287377 |
| 467 | 0.005177 | 0.025637 | 0.884542 |
| 469 | 0.00431 | 0.025306 | 1.013611 |
| 470 | 0.004249 | 0.025992 | 0.811355 |
| 471 | 0.004548 | 0.027775 | 0.99103 |
| 473 | 0.005266 | 0.027614 | 0.841048 |
| 474 | 0.005181 | 0.025263 | 1.045863 |
| 475 | 0.004214 | 0.024591 | 1.055417 |
| 476 | 0.004585 | 0.024957 | 0.96854 |
| 477 | 0.005587 | 0.026272 | 1.177024 |
| 478 | 0.005441 | 0.028445 | 0.988752 |
| 479 | 0.004717 | 0.027028 | 0.856542 |
| 480 | 0.004527 | 0.026333 | 0.732261 |
| 481 | 0.004189 | 0.026134 | 1.042274 |
| 482 | 0.004308 | 0.025592 | 1.144538 |
| 483 | 0.004232 | 0.025486 | 0.988084 |
| 484 | 0.005422 | 0.026264 | 0.930135 |
| 486 | 0.010488 | 0.036814 | 0.400608 |
| 487 | 0.009352 | 0.035227 | 0.398473 |
| 488 | 0.011628 | 0.039565 | 0.371609 |
| 489 | 0.010104 | 0.036386 | 0.522603 |
| 490 | 0.010243 | 0.035054 | 0.428049 |
| 491 | 0.009427 | 0.034974 | 0.456625 |
| 492 | 0.009978 | 0.034961 | 0.578826 |
| 493 | 0.017268 | 0.035854 | 0.439093 |
| 494 | 0.011939 | 0.036741 | 0.564414 |
| 495 | 0.009874 | 0.035139 | 0.393693 |
| 496 | 0.009436 | 0.033925 | 0.438625 |
| 499 | 0.017365 | 0.040412 | 0.408489 |
| 500 | 0.010443 | 0.035109 | 0.376297 |
| 501 | 0.098725 | 0.035501 | 0.705679 |
| 502 | 0.009362 | 0.036715 | 0.497175 |
| 503 | 0.010364 | 0.039033 | 0.408634 |
| 504 | 0.01097 | 0.034864 | 0.429402 |
| 505 | 0.011296 | 0.045354 | 0.444601 |
| 506 | 0.009332 | 0.034205 | 0.450232 |
| 507 | 0.009893 | 0.052264 | 0.571042 |
| 508 | 0.009472 | 0.034109 | 0.533749 |
| 510 | 0.010046 | 0.035326 | 0.405723 |
| 511 | 0.009439 | 0.03423 | 0.345853 |
| 512 | 0.009106 | 0.034492 | 0.364057 |
| 513 | 0.013017 | 0.036592 | 0.399432 |
| 514 | 0.029114 | 0.157091 | 0.665907 |
| 515 | 0.009588 | 0.035852 | 0.430589 |
| 516 | 0.01053 | 0.034159 | 0.347145 |
| 517 | 0.011616 | 0.049112 | 0.479119 |
| 518 | 0.010441 | 0.034833 | 0.526338 |
| 519 | 0.012705 | 0.03856 | 0.465362 |
| 520 | 0.036111 | 0.066426 | 0.601134 |
| 521 | 0.009721 | 0.035602 | 0.5263 |
| 522 | 0.009626 | 0.034174 | 0.411501 |
| 523 | 0.010243 | 0.034049 | 0.461773 |
| 524 | 0.009466 | 0.03404 | 0.450292 |
| 525 | 0.009484 | 0.035115 | 0.428652 |
| 526 | 0.034651 | 0.049443 | 0.470629 |
| 527 | 0.010225 | 0.035086 | 0.474979 |
| 528 | 0.01019 | 0.0345 | 0.559636 |
| 529 | 0.0097 | 0.044281 | 0.512486 |
| 531 | 0.011152 | 0.035546 | 0.358218 |
| 532 | 0.017782 | 0.037115 | 0.476891 |
| 533 | 0.010199 | 0.033963 | 0.420376 |
| 534 | 0.009483 | 0.035329 | 0.29936 |
| 535 | 0.90231 | 0.680855 | 0.881043 |
| 537 | 0.010176 | 0.034227 | 0.547417 |
| 539 | 0.009638 | 0.034846 | 0.4356 |
| 540 | 0.00967 | 0.035308 | 0.379571 |
| 541 | 0.020438 | 0.064563 | 0.553054 |
| 542 | 0.0096 | 0.035474 | 0.46663 |
| 543 | 0.010103 | 0.036928 | 0.391608 |
| 544 | 0.031817 | 0.071379 | 0.405136 |
| 545 | 0.011419 | 0.044632 | 0.471581 |
| 546 | 0.009434 | 0.034118 | 0.633882 |
| 547 | 0.009671 | 0.034574 | 0.37907 |
| 548 | 0.009753 | 0.03389 | 0.425888 |
| 549 | 0.009679 | 0.034543 | 0.416178 |
| 550 | 0.010414 | 0.035565 | 0.382037 |
| 551 | 0.009573 | 0.034555 | 0.569187 |
| 553 | 0.010895 | 0.037061 | 0.452852 |
| 554 | 0.0252 | 0.106556 | 0.399393 |
| 556 | 0.009499 | 0.034102 | 0.463958 |
| 557 | 0.016537 | 0.050391 | 0.406694 |
| 558 | 0.029851 | 0.098052 | 0.422865 |
| 559 | 0.240194 | 0.948046 | 0.755382 |
| 560 | 0.023732 | 0.142099 | 0.410883 |
| 561 | 0.009951 | 0.034656 | 0.525667 |
| 562 | 0.023838 | 0.071971 | 0.547672 |
| 563 | 0.029476 | 0.233222 | 0.518497 |
| 564 | 0.009775 | 0.033845 | 0.432675 |
| 565 | 0.014326 | 0.034722 | 0.462568 |
| 567 | 0.062129 | 0.223441 | 0.412738 |
| 568 | 0.010126 | 0.035292 | 0.406865 |
| 569 | 0.010231 | 0.035076 | 0.671012 |
| 570 | 0.010125 | 0.035003 | 0.480816 |
| 571 | 0.010039 | 0.034758 | 0.495482 |
| 572 | 0.010531 | 0.045745 | 0.522281 |
| 573 | 0.025354 | 0.034962 | 0.419466 |
| 574 | 0.243552 | 0.556199 | 0.762058 |
| 575 | 0.015572 | 0.035785 | 0.437496 |
| 576 | 0.013035 | 0.038805 | 0.531242 |
| 577 | 0.032586 | 0.079404 | 0.417157 |
| 578 | 0.02313 | 0.063316 | 0.474352 |
| 579 | 0.065246 | 0.302516 | 0.543815 |
| 581 | 0.354729 | 0.62217 | 1.144543 |
| 582 | 0.033618 | 0.058613 | 0.615124 |
| 583 | 0.308377 | 0.394948 | 0.932255 |
| 585 | 0.006337 | 0.020183 | 0.315108 |
| 586 | 0.163191 | 0.081969 | 0.381663 |
| 587 | 0.005108 | 0.023774 | 0.224587 |
| 588 | 0.021841 | 0.05963 | 0.370248 |
| 589 | 0.046721 | 0.333438 | 0.27475 |
| 590 | 0.090307 | 0.279891 | 0.29317 |
| 591 | 0.028787 | 0.07838 | 0.325743 |
| 592 | 0.005702 | 0.020121 | 0.306412 |
| 593 | 0.0099 | 0.046476 | 0.246072 |
| 594 | 0.015627 | 0.043937 | 0.215799 |
| 595 | 0.004629 | 0.019544 | 0.251833 |
| 596 | 0.046096 | 0.243097 | 0.24242 |
| 597 | 0.724998 | 0.981484 | 0.844379 |
| 598 | 0.026838 | 0.114469 | 0.289663 |
| 599 | 0.005828 | 0.019718 | 0.257416 |
| 600 | 0.005909 | 0.019865 | 0.251511 |
| 601 | 0.00614 | 0.020424 | 0.288905 |
| 602 | 0.005997 | 0.020776 | 0.216972 |
| 603 | 0.004669 | 0.019593 | 0.244223 |
| 604 | 0.008771 | 0.03346 | 0.323606 |
| 605 | 0.006624 | 0.020724 | 0.274329 |
| 606 | 0.005853 | 0.020382 | 0.242918 |
| 607 | 0.007747 | 0.021643 | 0.255363 |
| 608 | 0.005712 | 0.019678 | 0.221593 |
| 609 | 0.256073 | 0.504568 | 0.599603 |
| 610 | 0.005913 | 0.020199 | 0.199839 |
| 611 | 0.00447 | 0.019293 | 0.258074 |
| 612 | 0.005724 | 0.019734 | 0.360177 |
| 613 | 0.005694 | 0.019843 | 0.280102 |
| 614 | 0.005742 | 0.019885 | 0.286104 |
| 615 | 0.005705 | 0.019587 | 0.226829 |
| 616 | 0.006013 | 0.020573 | 0.266788 |
| 617 | 0.035272 | 0.094008 | 0.237734 |
| 618 | 0.026309 | 0.025971 | 0.242108 |
| 619 | 0.004687 | 0.020401 | 0.240359 |
| 620 | 0.00571 | 0.019825 | 0.218814 |
| 621 | 0.006001 | 0.020668 | 0.280146 |
| 622 | 0.082002 | 0.273485 | 0.335066 |
| 623 | 0.006029 | 0.020465 | 0.261982 |
| 624 | 0.140559 | 0.424388 | 0.42443 |
| 625 | 0.005826 | 0.019866 | 0.264531 |
| 626 | 0.051468 | 0.032244 | 0.306101 |
| 627 | 0.075104 | 0.276951 | 0.298932 |
| 628 | 0.005997 | 0.022048 | 0.26821 |
| 629 | 0.034034 | 0.401114 | 0.435605 |
| 630 | 0.011946 | 0.027566 | 0.254444 |
| 631 | 0.005914 | 0.020147 | 0.288981 |
| 632 | 0.037385 | 0.026122 | 0.362523 |
| 633 | 0.005826 | 0.019832 | 0.356419 |
| 634 | 0.010026 | 0.030547 | 0.206994 |
| 635 | 0.01508 | 0.04919 | 0.264614 |
| 636 | 0.296551 | 1.260655 | 0.610765 |
| 637 | 0.032124 | 0.375722 | 0.306647 |
| 638 | 0.005896 | 0.021846 | 0.271964 |
| 639 | 0.018881 | 0.148987 | 0.215144 |
| 640 | 0.018015 | 0.131161 | 0.288994 |
| 641 | 0.032334 | 0.02755 | 0.409355 |
| 642 | 0.067149 | 0.273403 | 0.35373 |
| 643 | 0.094609 | 0.53261 | 0.4944 |
| 644 | 0.006418 | 0.02472 | 0.231759 |
| 645 | 0.131335 | 0.734794 | 0.553125 |
| 646 | 0.029427 | 0.344127 | 0.390884 |
| 647 | 0.023863 | 0.136043 | 0.300959 |
| 650 | 0.010281 | 0.03014 | 0.242733 |
| 651 | 0.007994 | 0.028947 | 0.25194 |
| 652 | 0.15318 | 0.627023 | 0.43527 |
| 653 | 0.118823 | 0.69422 | 0.40527 |
| 654 | 0.105353 | 0.646828 | 0.388119 |
| 656 | 0.006783 | 0.02363 | 0.269316 |
| 657 | 0.307363 | 1.189775 | 0.605584 |
| 658 | 0.142732 | 0.450462 | 0.421859 |
| 659 | 0.22685 | 0.512994 | 0.582274 |
| 660 | 0.088314 | 0.402555 | 0.524885 |
| 661 | 0.180276 | 0.439707 | 0.530249 |
| 662 | 0.010698 | 0.060157 | 0.272901 |
| 663 | 0.161736 | 0.431703 | 0.609228 |
| 664 | 0.104153 | 0.348438 | 0.502663 |
| 665 | 0.509021 | 0.807128 | 0.717144 |
| 666 | 0.474224 | 0.60482 | 0.829418 |
| 667 | 0.04441 | 0.198916 | 0.289447 |
| 668 | 0.072205 | 0.466606 | 0.40305 |
| 669 | 0.026334 | 0.185174 | 0.304685 |
| 670 | 0.108713 | 0.572116 | 0.361198 |
| 671 | 0.054216 | 0.293384 | 0.286724 |
| 672 | 0.089022 | 0.461053 | 0.683679 |
| 673 | 0.052496 | 0.22393 | 0.290818 |
| 674 | 0.242917 | 0.426446 | 0.671781 |
| 675 | 0.020806 | 0.100778 | 0.254393 |
| 676 | 0.120498 | 0.594332 | 0.557211 |
| 677 | 0.083653 | 0.221249 | 0.399649 |
| 678 | 0.006713 | 0.026074 | 0.284843 |
| 679 | 0.012988 | 0.238136 | 0.251857 |
| 680 | 0.939588 | 1.168014 | 0.89748 |
| 682 | 0.116101 | 0.51627 | 0.411418 |
| 683 | 0.091835 | 0.41856 | 0.330861 |
| 684 | 0.269272 | 0.956985 | 0.388996 |
| 685 | 0.068553 | 0.420129 | 0.219238 |
| 686 | 0.118729 | 0.627236 | 0.408928 |
| 687 | 0.109438 | 0.486295 | 0.332833 |
| 688 | 0.136059 | 0.604523 | 0.382106 |
| 689 | 0.029476 | 0.230011 | 0.246326 |
| 690 | 0.713106 | 1.287446 | 0.527058 |
| 691 | 0.237497 | 0.831741 | 0.595391 |
| 692 | 0.223793 | 1.114774 | 0.464139 |
| 693 | 0.271839 | 2.152616 | 0.674694 |
| 694 | 0.05139 | 0.613276 | 0.240255 |
| 695 | 0.327666 | 1.036856 | 0.542561 |
| 696 | 0.098363 | 0.604772 | 0.25928 |
| 697 | 0.375897 | 0.957958 | 0.549728 |
| 698 | 0.026512 | 0.225899 | 0.241871 |
| 699 | 0.497694 | 1.50769 | 0.574673 |
| 700 | 0.288469 | 0.915024 | 0.510727 |
| 701 | 0.437509 | 1.020715 | 0.734873 |
| 702 | 0.046205 | 0.476705 | 0.190078 |
| 703 | 0.122863 | 0.83862 | 0.282447 |
| 704 | 0.147205 | 0.901082 | 0.301388 |
| 705 | 0.223074 | 0.853951 | 0.40577 |
| 706 | 0.008438 | 0.033092 | 0.191812 |
| 707 | 0.394494 | 0.997947 | 0.843619 |
| 708 | 0.186993 | 1.051411 | 0.468843 |
| 709 | 0.108893 | 0.601642 | 0.210326 |
| 710 | 0.091753 | 0.734575 | 0.237951 |
| 711 | 0.345755 | 1.159082 | 0.385849 |
| 712 | 0.115713 | 0.513471 | 0.287018 |
| 713 | 0.122493 | 0.686785 | 0.347889 |
| 714 | 0.191872 | 0.830288 | 0.567754 |
| 715 | 0.016321 | 0.055715 | 0.200218 |
| 716 | 0.020481 | 0.530732 | 0.198959 |
| 717 | 0.039274 | 0.234164 | 0.199885 |
| 718 | 0.344223 | 1.062216 | 0.47539 |
| 719 | 0.034917 | 0.299112 | 0.221854 |
| 720 | 0.149534 | 0.777313 | 0.356745 |
| 721 | 0.35108 | 1.01378 | 0.464163 |
| 722 | 0.234439 | 0.884539 | 0.353844 |
| 723 | 0.0088 | 0.035731 | 0.175315 |
| 724 | 0.302881 | 1.254204 | 0.495122 |
| 725 | 0.05075 | 0.39608 | 0.275113 |
| 726 | 0.081523 | 0.495768 | 0.301285 |
| 727 | 0.043541 | 0.247525 | 0.249821 |
| 728 | 0.33696 | 1.546341 | 0.460674 |
| 729 | 0.359043 | 1.008995 | 0.546047 |
| 730 | 0.097955 | 0.496152 | 0.329261 |
| 731 | 0.072919 | 0.443675 | 0.274429 |
| 732 | 0.015001 | 0.060524 | 0.182808 |
| 733 | 0.453244 | 1.410075 | 0.629982 |
| 734 | 0.342572 | 1.700432 | 0.413281 |
| 735 | 0.012613 | 0.050176 | 0.182816 |
| 736 | 0.016461 | 0.063952 | 0.182823 |
| 738 | 0.482005 | 1.605961 | 0.567678 |
| 739 | 0.58218 | 1.397435 | 0.754253 |
| 740 | 0.029093 | 0.051791 | 0.207152 |
| 741 | 0.349249 | 1.283688 | 0.511103 |
| 742 | 0.04606 | 0.482528 | 0.221214 |
| 743 | 0.141882 | 0.376925 | 2.230581 |
| 744 | 0.254656 | 0.939146 | 0.493241 |
| 745 | 0.015338 | 0.058975 | 0.213153 |
| 746 | 0.340833 | 0.962798 | 0.521323 |
| 747 | 0.701684 | 1.619727 | 0.658665 |
| 748 | 0.442992 | 1.472323 | 0.774321 |
| 749 | 0.196686 | 0.71019 | 0.441595 |
| 750 | 0.00964 | 0.038536 | 0.188239 |
| 751 | 0.360595 | 1.364262 | 0.554156 |
| 752 | 0.32576 | 1.182967 | 0.622747 |
| 753 | 0.066818 | 0.928007 | 0.240297 |
| 754 | 0.017628 | 0.16402 | 0.222692 |
| 755 | 0.237297 | 1.072629 | 0.451982 |
| 756 | 0.178168 | 1.009049 | 0.452329 |
| 757 | 0.012459 | 0.041059 | 0.194411 |
| 758 | 0.007271 | 0.030828 | 0.157203 |
| 759 | 0.031242 | 0.159577 | 0.186227 |
| 761 | 0.043353 | 0.146524 | 0.225515 |
| 762 | 0.007574 | 0.030242 | 0.208583 |
| 763 | 0.008587 | 0.032455 | 0.1986 |
| 764 | 0.008102 | 0.033408 | 0.169696 |
| 765 | 0.008951 | 0.03922 | 0.17488 |
| 766 | 0.018024 | 0.295069 | 0.205724 |
| 767 | 0.124411 | 0.768264 | 0.418591 |
| 768 | 0.008208 | 0.036795 | 0.183375 |
| 769 | 0.036346 | 0.37833 | 0.238709 |
| 770 | 0.279379 | 1.535747 | 0.723481 |
| 771 | 0.643475 | 0.520238 | 0.45387 |
| 772 | 0.014665 | 0.047355 | 0.166823 |
| 773 | 0.468419 | 2.022633 | 0.735404 |
| 774 | 0.362808 | 1.622526 | 0.436049 |
| 775 | 0.008859 | 0.038937 | 0.134732 |
| 776 | 0.311065 | 0.985297 | 0.574464 |
| 777 | 0.039862 | 0.065683 | 0.181863 |
| 779 | 0.123206 | 0.450318 | 0.697269 |
| 780 | 0.006814 | 0.029606 | 0.249389 |
| 781 | 0.009967 | 0.031473 | 0.286858 |
| 782 | 0.777497 | 1.325239 | 0.830948 |
| 783 | 0.587889 | 1.292049 | 1.019843 |
| 784 | 0.387894 | 0.821784 | 0.97539 |
| 786 | 0.120025 | 0.185762 | 0.380872 |
| 787 | 0.235698 | 0.942409 | 1.240669 |
| 788 | 0.336218 | 1.269178 | 0.957619 |
| 789 | 0.01732 | 0.096762 | 0.304029 |
| 791 | 0.617079 | 1.629033 | 1.466181 |
| 792 | 0.185997 | 0.704769 | 0.536405 |
| 793 | 0.877167 | 1.831368 | 1.454354 |
| 794 | 0.009408 | 0.076964 | 0.311169 |
| 795 | 0.099921 | 0.429592 | 0.632075 |
| 796 | 0.094809 | 0.536362 | 0.432216 |
| 797 | 0.244954 | 0.482612 | 0.621421 |
| 798 | 0.017916 | 0.084297 | 0.371982 |
| 799 | 0.012818 | 0.047289 | 0.29833 |
| 800 | 0.011675 | 0.13481 | 0.297163 |
| 801 | 0.099266 | 0.273608 | 0.403125 |
| 802 | 0.367887 | 1.18793 | 0.85394 |
| 803 | 0.006742 | 0.027036 | 0.289538 |
| 804 | 0.059456 | 0.162059 | 0.353715 |
| 805 | 0.01413 | 0.033509 | 0.294557 |
| 806 | 0.518086 | 1.610542 | 0.760268 |
| 807 | 0.022595 | 0.066194 | 0.294225 |
| 808 | 0.459064 | 1.277287 | 1.042237 |
| 810 | 0.012544 | 0.05468 | 0.274167 |
| 811 | 0.151174 | 0.538166 | 0.417778 |
| 812 | 0.006597 | 0.025521 | 0.283047 |
| 813 | 0.00961 | 0.027888 | 0.286255 |
| 814 | 0.048257 | 0.855988 | 0.357851 |
| 815 | 0.008921 | 0.03568 | 0.252635 |
| 816 | 0.39553 | 1.021138 | 0.918671 |
| 817 | 0.123764 | 0.912411 | 0.462477 |
| 818 | 0.007052 | 0.060048 | 0.259581 |
| 820 | 0.595672 | 0.572794 | 2.367059 |
| 821 | 0.027009 | 0.077125 | 0.292614 |
| 822 | 0.006996 | 0.035426 | 0.27968 |
| 823 | 0.61953 | 2.009281 | 0.808006 |
| 824 | 0.078118 | 0.029309 | 0.329722 |
| 825 | 0.038471 | 0.505871 | 0.321534 |
| 826 | 0.214307 | 1.236203 | 0.710454 |
| 827 | 0.187119 | 1.091001 | 0.678169 |
| 828 | 0.008155 | 0.032836 | 0.286114 |
| 829 | 0.043091 | 0.374192 | 0.385331 |
| 830 | 0.227057 | 0.993627 | 0.956788 |
| 831 | 0.111249 | 0.782791 | 0.514752 |
| 832 | 0.007741 | 0.034744 | 0.240929 |
| 833 | 0.148308 | 1.302523 | 0.621108 |
| 834 | 0.050544 | 0.635099 | 0.432155 |
| 835 | 0.107654 | 0.475431 | 0.419748 |
| 836 | 0.332776 | 1.197718 | 0.712195 |
| 837 | 0.007024 | 0.02723 | 0.249139 |
| 839 | 0.541139 | 1.487877 | 1.280705 |
| 840 | 0.434719 | 1.514761 | 1.27396 |
| 841 | 0.268407 | 1.048218 | 0.711256 |
| 842 | 0.258458 | 1.028083 | 0.818548 |
| 843 | 0.211266 | 0.691019 | 1.864166 |
| 846 | 0.14662 | 0.821729 | 1.1122 |
| 847 | 0.386867 | 1.568454 | 0.669131 |
| 848 | 0.080316 | 0.56734 | 0.850772 |
| 849 | 0.25383 | 1.721732 | 0.602704 |
| 850 | 0.526909 | 1.78363 | 0.891613 |
| 851 | 0.116222 | 0.76078 | 0.77039 |
| 852 | 0.206702 | 0.560921 | 1.415325 |
| 853 | 0.238304 | 1.081284 | 0.677935 |
| 854 | 0.053709 | 0.430447 | 0.580549 |
| 855 | 0.218825 | 0.941552 | 0.664315 |
| 856 | 0.101452 | 0.224533 | 0.577448 |
| 857 | 0.417144 | 0.902022 | 1.306233 |
| 858 | 0.03876 | 0.729869 | 0.32705 |
| 859 | 0.007745 | 0.034045 | 0.25266 |
| 860 | 0.008348 | 0.045165 | 0.274574 |
| 861 | 0.594983 | 1.639781 | 1.271579 |
| 862 | 0.900769 | 1.57937 | 1.004981 |
| 863 | 0.537996 | 1.331377 | 1.261956 |
| 864 | 0.388698 | 0.891772 | 1.285634 |
| 865 | 0.264661 | 1.079879 | 0.864063 |
| 866 | 0.06028 | 0.22964 | 0.478783 |
| 867 | 0.239258 | 0.716616 | 0.710261 |
| 868 | 0.008279 | 0.030116 | 0.264729 |
| 870 | 0.345182 | 0.941026 | 1.084312 |
| 871 | 0.381332 | 0.702232 | 1.235897 |
| 872 | 0.007184 | 0.0288 | 0.280018 |
| 873 | 0.011191 | 0.037242 | 0.285839 |
| 874 | 0.149494 | 0.617071 | 1.476994 |
| 875 | 0.172408 | 0.907391 | 0.867952 |
| 876 | 0.462175 | 1.088865 | 1.306636 |
| 878 | 0.010338 | 0.034189 | 0.216719 |
| 879 | 0.004925 | 0.019286 | 0.108567 |
| 881 | 0.005183 | 0.020939 | 0.088123 |
| 882 | 0.005094 | 0.020658 | 0.090945 |
| 883 | 0.005435 | 0.022801 | 0.085715 |
| 884 | 0.150855 | 0.669518 | 0.216276 |
| 885 | 0.352227 | 0.729722 | 0.522431 |
| 886 | 0.00519 | 0.04764 | 0.094334 |
| 887 | 0.006618 | 0.067628 | 0.098385 |
| 888 | 0.689507 | 1.833727 | 0.663895 |
| 889 | 0.192102 | 1.196074 | 0.344485 |
| 890 | 0.348046 | 1.61085 | 0.692815 |
| 891 | 0.080898 | 0.580847 | 0.200646 |
| 892 | 0.044425 | 0.718329 | 0.121203 |
| 893 | 0.382721 | 0.843293 | 0.376432 |
| 894 | 0.068781 | 0.640743 | 0.21479 |
| 896 | 0.014415 | 0.055218 | 0.107079 |
| 897 | 0.00687 | 0.027706 | 0.094585 |
| 898 | 0.286212 | 1.357183 | 0.357932 |
| 899 | 0.297653 | 1.1528 | 0.302755 |
| 900 | 0.327981 | 0.932419 | 0.386499 |
| 901 | 0.010727 | 0.05943 | 0.088041 |
| 902 | 0.350484 | 1.180504 | 0.40489 |
| 903 | 0.374566 | 1.622565 | 0.396166 |
| 904 | 0.166397 | 1.049689 | 0.320325 |
| 905 | 0.136541 | 0.817688 | 0.362347 |
| 906 | 0.291429 | 1.325833 | 0.396889 |
| 907 | 0.347797 | 1.267447 | 0.541789 |
| 908 | 0.034779 | 0.467747 | 0.163031 |
| 909 | 0.164487 | 0.350343 | 0.141732 |
| 910 | 0.265078 | 0.886219 | 0.407203 |
| 911 | 0.220201 | 0.717038 | 0.423402 |
| 912 | 0.186388 | 0.85741 | 0.362314 |
| 913 | 0.518467 | 1.762848 | 0.547938 |
| 914 | 0.40602 | 1.766605 | 0.454544 |
| 915 | 0.163459 | 1.042566 | 0.26138 |
| 916 | 0.296433 | 1.092387 | 0.371475 |
| 917 | 0.635553 | 1.261914 | 0.549559 |
| 918 | 0.291057 | 1.006901 | 0.474071 |
| 919 | 0.413799 | 1.246473 | 0.564896 |
| 920 | 0.179531 | 1.451147 | 0.495419 |
| 921 | 0.178932 | 1.285129 | 0.41161 |
| 923 | 0.317288 | 1.146979 | 0.387911 |
| 924 | 0.273164 | 0.914613 | 0.414757 |
| 925 | 0.19808 | 0.725391 | 0.296935 |
| 926 | 0.009777 | 0.035042 | 0.100935 |
| 927 | 0.436574 | 1.262457 | 0.683576 |
| 928 | 0.183262 | 0.813709 | 0.402288 |
| 929 | 0.094365 | 0.787482 | 0.213812 |
| 930 | 0.009025 | 0.063975 | 0.099171 |
| 931 | 0.032034 | 0.095047 | 0.131713 |
| 932 | 0.429418 | 1.050149 | 0.621605 |
| 933 | 0.0172 | 0.052524 | 0.099638 |
| 934 | 0.632772 | 1.169765 | 0.781487 |
| 935 | 0.253132 | 1.3481 | 0.278776 |
| 936 | 0.170298 | 1.000176 | 0.277753 |
| 937 | 0.15824 | 0.84717 | 0.442072 |
| 938 | 0.20538 | 0.88334 | 0.432407 |
| 939 | 0.224863 | 0.999881 | 0.380371 |
| 940 | 0.006178 | 0.021918 | 0.089462 |
| 941 | 0.011284 | 0.066173 | 0.115573 |
| 942 | 0.173491 | 0.708929 | 0.733394 |
| 943 | 0.189128 | 1.089849 | 0.316464 |
| 944 | 0.090245 | 0.422225 | 0.480858 |
| 946 | 0.230236 | 0.894889 | 0.396333 |
| 947 | 0.257151 | 0.909063 | 0.325964 |
| 948 | 0.170075 | 0.588195 | 0.260671 |
| 949 | 0.187726 | 0.806307 | 0.331384 |
| 950 | 0.22905 | 0.942413 | 0.603096 |
| 951 | 0.397644 | 0.841753 | 0.3522 |
| 952 | 0.049028 | 0.367599 | 0.253907 |
| 953 | 0.147509 | 0.791804 | 0.804695 |
| 954 | 0.16991 | 0.798194 | 0.475492 |
| 955 | 0.264691 | 0.591625 | 0.406549 |
| 957 | 0.148218 | 0.602345 | 0.46558 |
| 958 | 0.204506 | 0.863187 | 0.592641 |
| 959 | 0.255224 | 1.064249 | 0.774381 |
| 961 | 0.156063 | 1.110527 | 0.516403 |
| 962 | 0.212997 | 1.217959 | 0.365859 |
| 963 | 0.0211 | 0.144681 | 0.150198 |
| 964 | 0.489565 | 1.57383 | 0.615012 |
| 965 | 0.251985 | 0.770085 | 0.522089 |
| 966 | 0.55303 | 0.923252 | 0.527881 |
| 967 | 0.374958 | 1.224169 | 0.526617 |
| 968 | 0.07674 | 0.128785 | 0.431369 |
| 969 | 0.186199 | 0.698537 | 0.472067 |
| 970 | 0.161868 | 0.595542 | 0.889933 |
| 971 | 0.057774 | 0.497863 | 0.206401 |
| 972 | 0.089708 | 0.555687 | 0.328706 |
| 974 | 0.4611 | 1.588992 | 0.988689 |
| 975 | 0.501721 | 1.70434 | 0.729741 |
| 976 | 0.468553 | 1.572864 | 0.520521 |
| 977 | 0.465145 | 1.349452 | 0.80622 |
| 978 | 0.060567 | 1.227042 | 0.179786 |
| 980 | 0.649997 | 2.424418 | 0.991253 |
| 981 | 0.675473 | 1.395536 | 0.799773 |
| 982 | 0.493201 | 1.634458 | 0.836638 |
| 983 | 0.6355 | 4.406334 | 0.657997 |
| 984 | 0.622337 | 2.861176 | 0.393731 |
| 985 | 0.247394 | 1.921413 | 0.406224 |
| 986 | 0.098809 | 1.089629 | 0.184354 |
| 987 | 0.47511 | 2.42364 | 0.361921 |
| 988 | 0.364262 | 1.773289 | 0.713466 |
| 989 | 0.751797 | 1.903482 | 0.467399 |
| 992 | 0.251144 | 1.61049 | 0.379502 |
| 993 | 0.209284 | 1.691842 | 0.44776 |
| 994 | 0.34175 | 2.039731 | 0.798924 |
| 995 | 0.560565 | 3.12071 | 0.388763 |
| 996 | 0.407642 | 3.358356 | 0.353555 |
| 997 | 0.930576 | 2.730102 | 0.497813 |
| 998 | 0.325541 | 2.234049 | 0.512623 |
| 999 | 0.37115 | 2.362841 | 0.625508 |
| 1000 | 0.200748 | 2.565779 | 0.285251 |
| 1001 | 0.101767 | 1.532869 | 0.325446 |
| 1002 | 0.334714 | 3.270477 | 0.575591 |
| 1003 | 0.294501 | 2.785205 | 0.192242 |
| 1004 | 0.42759 | 3.329744 | 0.366353 |
| 1005 | 0.658112 | 2.554948 | 0.265405 |
| 1006 | 0.657566 | 1.865741 | 1.037788 |
| 1007 | 0.428267 | 2.664938 | 0.294918 |
| 1008 | 0.123986 | 1.595956 | 0.302035 |
| 1009 | 0.274442 | 2.817702 | 0.287299 |
| 1010 | 0.238204 | 3.007669 | 0.358667 |
| 1011 | 0.329633 | 2.440677 | 0.33232 |
| 1012 | 0.374152 | 3.023523 | 0.243111 |
| 1013 | 0.040086 | 0.407359 | 0.097212 |
| 1014 | 0.650383 | 2.577328 | 0.702306 |
| 1015 | 0.556326 | 3.822954 | 0.220832 |
| 1016 | 0.286549 | 2.359215 | 0.398523 |
| 1017 | 0.391716 | 5.493401 | 0.257803 |
| 1018 | 0.232929 | 2.135935 | 0.649378 |
| 1019 | 0.28219 | 2.371224 | 0.266729 |
| 1020 | 0.51863 | 2.875937 | 0.340747 |
| 1021 | 0.330813 | 1.726956 | 0.254804 |
| 1022 | 0.017793 | 0.063328 | 0.09479 |
| 1023 | 0.632422 | 5.001505 | 0.355245 |
| 1024 | 0.18462 | 1.771516 | 0.207356 |
| 1025 | 0.252709 | 2.019318 | 0.193805 |
| 1026 | 0.266125 | 3.425025 | 0.244887 |
| 1027 | 0.333355 | 2.88353 | 0.287246 |
| 1028 | 0.279444 | 1.656027 | 0.326932 |
| 1029 | 0.757301 | 2.168554 | 0.177607 |
| 1030 | 0.787768 | 3.103594 | 0.962656 |
| 1031 | 0.485323 | 3.727265 | 0.678268 |
| 1032 | 0.152334 | 1.921508 | 0.428204 |
| 1033 | 0.10525 | 1.592774 | 0.153351 |
| 1034 | 0.017448 | 0.092714 | 0.088247 |
| 1035 | 0.32027 | 2.717977 | 0.197946 |
| 1036 | 0.315469 | 1.59956 | 0.232083 |
| 1037 | 0.583145 | 2.318094 | 0.166805 |
| 1039 | 0.347433 | 2.874902 | 0.471893 |
| 1040 | 0.142964 | 1.920848 | 0.351992 |
| 1042 | 0.202209 | 2.100867 | 0.315035 |
| 1044 | 0.349521 | 2.320673 | 0.253838 |
| 1045 | 0.210983 | 0.761205 | 0.187876 |
| 1046 | 0.090748 | 0.371038 | 0.186849 |
| 1048 | 0.066928 | 0.667905 | 0.236017 |
| 1049 | 0.019242 | 0.114164 | 0.097972 |
| 1051 | 0.317465 | 2.158948 | 0.221567 |
| 1053 | 0.034762 | 0.16757 | 0.098127 |
| 1054 | 1.115094 | 3.572908 | 0.107919 |
| 1055 | 0.426255 | 2.279363 | 0.875474 |
| 1056 | 0.35721 | 2.140493 | 0.659378 |
| 1058 | 0.55246 | 3.14052 | 0.533825 |
| 1059 | 0.282424 | 1.535033 | 0.458698 |
| 1060 | 0.508811 | 3.071866 | 0.445119 |
| 1061 | 0.525733 | 2.601345 | 0.545443 |
| 1062 | 0.723702 | 1.828112 | 0.521941 |
| 1063 | 0.699279 | 2.002368 | 1.090698 |
| 1064 | 0.488057 | 1.746524 | 0.694956 |
| 1065 | 0.525503 | 2.352257 | 0.927797 |
| 1066 | 0.718979 | 3.256654 | 0.939838 |
| 1067 | 0.491483 | 1.809573 | 0.745394 |
| 1068 | 1.179541 | 3.74017 | 0.467877 |
| 1069 | 0.680638 | 2.4638 | 0.562974 |
| 1071 | 0.209584 | 0.500858 | 0.031699 |
| 1072 | 0.427886 | 1.498126 | 0.045503 |
| 1074 | 0.225285 | 0.934492 | 0.092819 |
| 1075 | 0.137715 | 0.599395 | 0.037514 |
| 1076 | 0.218044 | 1.024494 | 0.018691 |
| 1077 | 0.357504 | 1.710835 | 0.077848 |
| 1078 | 0.446448 | 1.518153 | 0.329426 |
| 1079 | 0.068825 | 0.57375 | 0.014851 |
| 1080 | 0.003884 | 0.019354 | 0.011699 |
| 1081 | 0.132094 | 0.892676 | 0.083692 |
| 1082 | 0.11434 | 0.923226 | 0.056705 |
| 1083 | 0.048631 | 0.398633 | 0.030346 |
| 1084 | 0.09557 | 0.924719 | 0.027728 |
| 1085 | 0.319794 | 0.776496 | 0.0123 |
| 1086 | 0.841477 | 2.348773 | 0.384012 |
| 1087 | 0.52158 | 1.199952 | 0.055537 |
| 1088 | 0.078823 | 0.411586 | 0.028465 |
| 1089 | 0.00343 | 0.019524 | 0.010609 |
| 1090 | 0.003436 | 0.019498 | 0.011946 |
| 1091 | 0.102675 | 0.821406 | 0.058983 |
| 1092 | 0.106604 | 0.815445 | 0.072592 |
| 1093 | 0.058575 | 0.476647 | 0.055827 |
| 1094 | 0.582942 | 1.352039 | 0.521226 |
| 1095 | 0.274994 | 0.998033 | 0.05091 |
| 1096 | 0.135482 | 0.659134 | 0.082443 |
| 1097 | 0.092301 | 0.934711 | 0.104868 |
| 1098 | 0.128824 | 1.226146 | 0.0726 |
| 1099 | 0.053711 | 0.584595 | 0.057494 |
| 1100 | 0.06731 | 0.735897 | 0.070774 |
| 1101 | 0.112476 | 0.815231 | 0.062219 |
| 1102 | 0.415663 | 1.212428 | 0.079545 |
| 1103 | 0.412005 | 1.754218 | 0.07277 |
| 1104 | 0.094334 | 0.470474 | 0.097149 |
| 1105 | 0.028017 | 0.537236 | 0.024877 |
| 1106 | 0.053871 | 0.742137 | 0.06444 |
| 1107 | 0.095169 | 1.103475 | 0.065829 |
| 1108 | 0.093046 | 0.820538 | 0.035385 |
| 1109 | 0.057641 | 0.482332 | 0.044518 |
| 1110 | 0.455043 | 1.55682 | 0.197962 |
| 1111 | 0.383534 | 1.350223 | 0.034935 |
| 1112 | 0.069077 | 0.719701 | 0.057509 |
| 1113 | 0.039422 | 0.485263 | 0.043295 |
| 1114 | 0.067072 | 0.91085 | 0.025613 |
| 1115 | 0.039005 | 0.503229 | 0.02457 |
| 1116 | 0.033876 | 0.323061 | 0.033704 |
| 1117 | 0.064028 | 0.56885 | 0.033787 |
| 1118 | 0.250182 | 0.508475 | 0.021224 |
| 1119 | 0.37371 | 0.826558 | 0.089181 |
| 1120 | 0.090101 | 0.592721 | 0.06047 |
| 1121 | 0.059672 | 1.02895 | 0.070457 |
| 1122 | 0.02492 | 0.538694 | 0.02872 |
| 1125 | 0.122305 | 0.680083 | 0.045805 |
| 1126 | 0.463398 | 1.267668 | 0.085815 |
| 1127 | 0.238281 | 1.080024 | 0.095938 |
| 1128 | 0.073365 | 0.521087 | 0.063778 |
| 1129 | 0.031089 | 0.393886 | 0.022686 |
| 1130 | 0.093374 | 0.767258 | 0.115614 |
| 1131 | 0.079149 | 0.574318 | 0.048331 |
| 1132 | 0.074191 | 0.643954 | 0.023463 |
| 1133 | 0.062971 | 0.480284 | 0.031931 |
| 1134 | 10.216714 | 2.20506 | 0.132402 |
| 1135 | 0.256515 | 0.844706 | 0.057365 |
| 1136 | 0.10945 | 0.887249 | 0.110266 |
| 1137 | 0.029429 | 0.352312 | 0.033672 |
| 1138 | 0.100706 | 0.864622 | 0.084684 |
| 1139 | 0.068952 | 0.846332 | 0.073941 |
| 1140 | 0.088214 | 0.797631 | 0.063666 |
| 1141 | 0.060094 | 0.359254 | 0.051047 |
| 1142 | 0.449206 | 1.015907 | 0.520133 |
| 1143 | 0.015059 | 0.08374 | 0.013579 |
| 1144 | 0.021431 | 0.415068 | 0.029096 |
| 1145 | 0.065197 | 0.376581 | 0.059013 |
| 1146 | 0.091757 | 0.625511 | 0.054384 |
| 1147 | 0.069289 | 0.534903 | 0.093464 |
| 1148 | 0.09357 | 0.984551 | 0.073067 |
| 1149 | 0.087768 | 0.722494 | 0.041795 |
| 1150 | 0.371622 | 1.089698 | 0.054021 |
| 1151 | 0.2795 | 0.947429 | 0.047765 |
| 1152 | 0.107195 | 0.384755 | 0.042152 |
| 1153 | 0.170766 | 0.881601 | 0.079923 |
| 1154 | 0.165804 | 1.199739 | 0.082085 |
| 1155 | 0.133725 | 0.619659 | 0.096667 |
| 1156 | 0.083408 | 0.615919 | 0.018728 |
| 1157 | 0.005065 | 0.022682 | 0.011868 |
| 1158 | 0.279768 | 0.567474 | 0.470594 |
| 1159 | 0.366023 | 1.092993 | 0.087006 |
| 1160 | 0.27576 | 0.987157 | 0.055226 |
| 1161 | 0.00888 | 0.211542 | 0.013514 |
| 1162 | 0.321948 | 1.123212 | 0.077234 |
| 1163 | 0.238473 | 0.676024 | 0.108033 |
| 1164 | 0.174887 | 0.553471 | 0.031922 |
| 1165 | 0.362907 | 1.060364 | 0.105512 |
| 1167 | 0.440301 | 3.41734 | |
| 1168 | 2.269595 | 6.148748 | |
| 1169 | 1.81238 | 4.676323 | |
| 1170 | 0.205013 | 0.821177 | |
| 1171 | 1.757897 | 3.916135 | |
| 1172 | 1.485171 | 1.939494 | |
| 1173 | 0.294994 | 0.522214 | |
| 1174 | 0.375652 | 1.011377 | |
| 1175 | 0.751857 | 4.08474 | |
| 1176 | 0.543517 | 3.338898 | |
| 1177 | 1.544519 | 7.245177 | |
| 1178 | 0.736878 | 2.629057 | |
| 1179 | 0.686547 | 2.072089 | |
| 1180 | 0.237255 | 0.98724 | |
| 1182 | 0.654624 | 1.237891 | |
| 1183 | 0.909645 | 4.061809 | |
| 1184 | 0.544952 | 2.024292 | |
| 1185 | 0.147863 | 1.25923 | |
| 1186 | 0.434429 | 1.954991 | |
| 1187 | 0.505786 | 3.505564 | |
| 1188 | 0.406861 | 2.548182 | |
| 1189 | 0.4624 | 2.051662 | |
| 1190 | 0.071895 | 0.226536 | |
| 1191 | 0.02375 | 0.087245 | |
| 1192 | 1.717893 | 7.465639 | |
| 1193 | 0.542623 | 4.373519 | |
| 1194 | 0.354838 | 3.577394 | |
| 1195 | 0.259933 | 3.12244 | |
| 1196 | 0.215423 | 2.052223 | |
| 1197 | 0.526893 | 2.109184 | |
| 1198 | 0.822275 | 1.940819 | |
| 1199 | 2.037406 | 9.409115 | |
| 1200 | 1.095359 | 5.130504 | |
| 1201 | 0.157644 | 0.731059 | |
| 1202 | 0.230264 | 1.923086 | |
| 1204 | 0.044579 | 0.913551 | |
| 1206 | 0.511955 | 1.209704 | |
| 1207 | 0.046192 | 0.162052 | |
| 1208 | 0.026908 | 0.124359 | |
| 1209 | 0.281822 | 3.216097 | |
| 1210 | 0.220147 | 2.427366 | |
| 1211 | 0.111229 | 1.298379 | |
| 1213 | 0.141412 | 0.865279 | |
| 1214 | 0.15488 | 0.459765 | |
| 1215 | 0.997818 | 3.321803 | |
| 1216 | 1.025901 | 4.406269 | |
| 1217 | 0.260845 | 2.930463 | |
| 1218 | 0.345315 | 3.143776 | |
| 1219 | 0.305255 | 1.957145 | |
| 1220 | 0.172212 | 1.168102 | |
| 1221 | 0.248967 | 0.814305 | |
| 1222 | 0.03732 | 0.119114 | |
| 1223 | 1.247387 | 3.024953 | |
| 1224 | 0.024175 | 0.081463 | |
| 1225 | 0.057454 | 0.455597 | |
| 1226 | 0.361586 | 4.237747 | |
| 1227 | 0.15804 | 1.518132 | |
| 1228 | 0.015375 | 0.056146 | |
| 1229 | 0.444241 | 1.249649 | |
| 1230 | 0.649691 | 1.15827 | |
| 1232 | 0.378509 | 0.925671 | |
| 1233 | 0.325411 | 2.625939 | |
| 1234 | 0.206621 | 2.051787 | |
| 1235 | 0.019165 | 0.09314 | |
| 1236 | 0.118642 | 1.658952 | |
| 1237 | 0.017709 | 0.057603 | |
| 1238 | 0.015895 | 0.043364 | |
| 1239 | 0.025104 | 0.079453 | |
| 1240 | 0.361175 | 2.493372 | |
| 1242 | 0.325351 | 1.821619 | |
| 1243 | 0.411323 | 2.267424 | |
| 1244 | 0.262681 | 1.368943 | |
| 1245 | 0.209877 | 1.009418 | |
| 1246 | 0.689115 | 0.935799 | |
| 1247 | 1.206727 | 3.45224 | |
| 1248 | 0.997846 | 3.186952 | |
| 1249 | 1.03331 | 4.151541 | |
| 1250 | 0.526962 | 1.830667 | |
| 1251 | 0.038789 | 0.160427 | |
| 1252 | 0.529876 | 1.476753 | |
| 1253 | 0.148526 | 0.512689 | |
| 1254 | 0.171619 | 0.496272 | |
| 1255 | 1.440028 | 4.650389 | |
| 1256 | 1.246323 | 3.526473 | |
| 1257 | 1.630471 | 3.27436 | |
| 1258 | 1.175351 | 3.563138 | |
| 1259 | 0.846546 | 1.714691 | |
| 1260 | 1.043862 | 0.869917 | |
| 1261 | 0.290872 | 0.44244 | |
| 1263 | 0.493679 | 0.657699 | 1.716926 |
| 1264 | 0.298742 | 0.443995 | 1.012966 |
| 1265 | 0.378744 | 0.471388 | 1.244712 |
| 1266 | 0.211218 | 0.327944 | 0.97188 |
| 1267 | 0.17315 | 0.41676 | 1.57404 |
| 1268 | 0.213092 | 0.396399 | 1.097609 |
| 1269 | 0.227781 | 0.372048 | 1.433456 |
| 1270 | 0.004499 | 0.018835 | 1.068802 |
| 1271 | 0.180553 | 0.407587 | 1.83541 |
| 1272 | 0.394666 | 0.599394 | 1.66783 |
| 1273 | 0.281898 | 0.489728 | 1.50982 |
| 1274 | 0.243913 | 0.319871 | 0.788908 |
| 1275 | 0.262209 | 0.4805 | 3.95034 |
| 1276 | 0.378342 | 0.610004 | 1.467933 |
| 1277 | 0.273883 | 0.469937 | 1.070136 |
| 1278 | 0.315846 | 0.734033 | 0.89974 |
| 1279 | 0.189246 | 0.354251 | 6.159851 |
| 1280 | 0.388472 | 0.494888 | 1.410746 |
| 1281 | 0.00514 | 0.020652 | 0.591781 |
| 1282 | 0.289013 | 0.482967 | 2.197797 |
| 1283 | 0.053841 | 0.067338 | 0.927451 |
| 1284 | 0.570301 | 0.633162 | 2.538618 |
| 1285 | 0.255977 | 0.464247 | 1.400939 |
| 1286 | 0.003738 | 0.01781 | 0.498459 |
| 1287 | 0.422493 | 0.771168 | 1.722777 |
| 1288 | 0.373076 | 0.512993 | 1.52444 |
| 1289 | 0.003707 | 0.017964 | 0.668949 |
| 1290 | 0.02527 | 0.048054 | 0.658627 |
| 1291 | 0.010359 | 0.025134 | 0.65155 |
| 1292 | 0.018398 | 0.043801 | 0.757339 |
| 1293 | 0.01987 | 0.051117 | 0.725236 |
| 1294 | 0.010602 | 0.029974 | 0.799556 |
| 1295 | 0.008196 | 0.027605 | 0.669465 |
| 1296 | 0.062396 | 0.233757 | 1.952551 |
| 1298 | 0.003269 | 0.017718 | 0.60053 |
| 1302 | 0.003654 | 0.017971 | 0.669614 |
| 1453 | 0.029035 | 0.074756 | 1.361452 |
| 1456 | 0.070888 | 0.141499 | 1.733833 |
| 1457 | 0.005302 | 0.02091 | 0.86533 |
| 1458 | 0.022487 | 0.056128 | 0.69949 |
| 1459 | 0.041481 | 0.10652 | 1.298416 |
| 1460 | 0.178585 | 0.283471 | 1.592463 |
| 1461 | 0.003819 | 0.018816 | 0.634444 |
| 1462 | 0.089803 | 0.219634 | 2.496717 |
| 1463 | 0.367361 | 0.489496 | 3.718475 |
| 1465 | 0.05509 | 0.079253 | 0.882252 |
| 1466 | 0.125624 | 0.195771 | 1.647541 |
| 1467 | 0.104653 | 0.186069 | 1.135532 |
| 1468 | 0.036721 | 0.09977 | 0.954748 |
| 1469 | 0.12058 | 0.220731 | 1.105286 |
| 1470 | 0.107829 | 0.199233 | 0.937104 |
| 1471 | 0.112351 | 0.216795 | 1.247919 |
| 1472 | 0.229734 | 0.29791 | 1.053188 |
| 1473 | 0.005236 | 0.020279 | 0.786991 |
| 1474 | 0.094218 | 0.183605 | 2.872396 |
| 1475 | 0.103852 | 0.178429 | 2.934125 |
| 1476 | 0.032997 | 0.071639 | 1.468191 |
| 1477 | 0.004763 | 0.019988 | 0.744601 |
| 1479 | 0.004862 | 0.02006 | 0.646639 |
| 1480 | 0.062493 | 0.120887 | 1.421179 |
| 1482 | 0.063512 | 0.117236 | 1.02612 |
| 1483 | 0.003687 | 0.018638 | 0.806893 |
| 1485 | 0.002923 | 0.016326 | 0.810278 |
| 1486 | 0.003046 | 0.016492 | 0.639067 |
| 1487 | 0.002997 | 0.016251 | 0.874065 |
| 1488 | 0.002931 | 0.015913 | 0.718561 |
| 1489 | 0.00278 | 0.016262 | 0.945829 |
| 1490 | 0.003396 | 0.017309 | 0.839493 |
| 1491 | 0.002835 | 0.016415 | 0.616515 |
| 1492 | 0.00337 | 0.017996 | 0.87573 |
| 1493 | 0.002838 | 0.016276 | 0.706791 |
| 1494 | 0.002936 | 0.016453 | 0.886075 |
| 1496 | 0.003021 | 0.016335 | 0.634456 |
| 1497 | 0.002921 | 0.016072 | 0.841508 |
| 1498 | 0.002932 | 0.016361 | 0.582778 |
| 1499 | 0.002819 | 0.017478 | 0.948983 |
| 1500 | 0.003006 | 0.017306 | 0.604048 |
| 1501 | 0.002807 | 0.016489 | 0.785628 |
| 1502 | 0.002769 | 0.015804 | 0.755524 |
| 1503 | 0.002882 | 0.016466 | 0.819839 |
| 1504 | 0.003412 | 0.016569 | 0.709579 |
| 1505 | 0.003997 | 0.017625 | 0.888899 |
| 1506 | 0.00289 | 0.017248 | 0.894677 |
| 1507 | 0.002795 | 0.01699 | 0.79213 |
| 1508 | 0.002911 | 0.016103 | 0.775025 |
| 1509 | 0.002935 | 0.016148 | 0.746427 |
| 1510 | 0.002847 | 0.016713 | 0.598686 |
| 1511 | 0.002895 | 0.016502 | 0.768356 |
| 1512 | 0.003954 | 0.017185 | 0.677016 |
| 1514 | 0.002815 | 0.027933 | 0.931244 |
| 1515 | 0.002933 | 0.027695 | 0.651567 |
| 1516 | 0.002785 | 0.026655 | 0.652685 |
| 1517 | 0.002783 | 0.027738 | 0.565322 |
| 1518 | 0.002828 | 0.026694 | 0.631511 |
| 1519 | 0.002834 | 0.027337 | 0.66479 |
| 1520 | 0.003453 | 0.028188 | 0.710203 |
| 1521 | 0.002992 | 0.026775 | 0.61482 |
| 1522 | 0.003072 | 0.028592 | 0.83538 |
| 1523 | 0.003473 | 0.028775 | 0.571612 |
| 1524 | 0.002805 | 0.027075 | 0.704035 |
| 1525 | 0.003615 | 0.030549 | 0.735642 |
| 1526 | 0.002812 | 0.027292 | 0.770641 |
| 1527 | 0.00283 | 0.026705 | 0.696095 |
| 1528 | 0.002794 | 0.026616 | 0.600694 |
| 1529 | 0.002883 | 0.026458 | 0.557503 |
| 1530 | 0.002978 | 0.028069 | 0.812791 |
| 1532 | 0.002984 | 0.027308 | 0.532545 |
| 1533 | 0.00285 | 0.02682 | 0.563657 |
| 1534 | 0.002846 | 0.02764 | 0.746323 |
| 1536 | 0.002799 | 0.027063 | 0.751124 |
| 1537 | 0.002812 | 0.02665 | 0.629611 |
| 1538 | 0.002753 | 0.026783 | 0.503323 |
| 1539 | 0.002909 | 0.027333 | 0.504615 |
| 1540 | 0.002819 | 0.027702 | 0.694792 |
| 1541 | 0.002759 | 0.02709 | 0.560466 |
| 1542 | 0.002733 | 0.027285 | 0.651773 |
| 1543 | 0.002765 | 0.026253 | 0.573015 |
| 1544 | 0.002706 | 0.027092 | 0.654941 |
| 1545 | 0.002874 | 0.026632 | 0.714128 |
| 1546 | 0.00294 | 0.027778 | 0.636466 |
| 1548 | 0.00279 | 0.028079 | 0.706656 |
| 1549 | 0.002731 | 0.027168 | 0.522207 |
| 1550 | 0.002677 | 0.027476 | 0.696415 |
| 1551 | 0.002816 | 0.027408 | 0.60935 |
| 1552 | 0.002864 | 0.027019 | 0.564324 |
| 1553 | 0.00289 | 0.027219 | 0.656982 |
| 1554 | 0.002904 | 0.027245 | 0.709589 |
| 1555 | 0.002857 | 0.027357 | 0.770251 |
| 1556 | 0.002779 | 0.027449 | 0.600841 |
| 1557 | 0.002804 | 0.027851 | 0.819866 |
| 1558 | 0.002822 | 0.027061 | 0.698614 |
| 1559 | 0.002789 | 0.026908 | 0.526565 |
| 1560 | 0.002867 | 0.02696 | 0.567773 |
| 1561 | 0.002954 | 0.026183 | 0.535325 |
| 1562 | 0.002832 | 0.028297 | 0.591001 |
| 1563 | 0.002851 | 0.026715 | 0.518507 |
| 1564 | 0.002773 | 0.027867 | 0.574878 |
| 1565 | 0.002829 | 0.027062 | 0.707218 |
| 1566 | 0.002787 | 0.027944 | 0.60274 |
| 1567 | 0.002789 | 0.026836 | 0.681669 |
| 1568 | 0.002819 | 0.026247 | 0.679288 |
| 1569 | 0.00288 | 0.026881 | 0.755689 |
| 1570 | 0.002853 | 0.026701 | 0.743775 |
| 1571 | 0.002811 | 0.027011 | 0.525869 |
| 1572 | 0.00285 | 0.027158 | 0.513429 |
| 1573 | 0.002801 | 0.027285 | 0.801479 |
| 1574 | 0.00283 | 0.026694 | 0.625502 |
| 1575 | 0.002879 | 0.026776 | 0.59054 |
| 1576 | 0.002797 | 0.026677 | 0.838621 |
| 1577 | 0.003048 | 0.027552 | 0.622979 |
| 1578 | 0.002889 | 0.027386 | 0.598139 |
| 1579 | 0.002955 | 0.027358 | 0.491017 |
| 1580 | 0.002857 | 0.026945 | 0.435188 |
| 1581 | 0.002782 | 0.02785 | 0.477584 |
| 1582 | 0.00277 | 0.026769 | 0.585209 |
| 1583 | 0.002882 | 0.027091 | 0.705566 |
| 1584 | 0.002823 | 0.026051 | 0.588066 |
| 1585 | 0.003011 | 0.02627 | 0.645734 |
| 1586 | 0.002783 | 0.027931 | 0.509146 |
| 1587 | 0.002834 | 0.027194 | 0.594226 |
| 1588 | 0.002788 | 0.027098 | 0.522658 |
| 1589 | 0.002909 | 0.027134 | 0.76665 |
| 1590 | 0.002772 | 0.026947 | 0.590476 |
| 1592 | 0.002841 | 0.026809 | 0.671333 |
| 1593 | 0.002895 | 0.026378 | 0.470406 |
| 1594 | 0.00308 | 0.026761 | 0.571625 |
| 1595 | 0.00292 | 0.026874 | 0.592748 |
| 1596 | 0.002734 | 0.02666 | 0.615701 |
| 1597 | 0.00288 | 0.026918 | 0.694078 |
| 1598 | 0.002796 | 0.028141 | 0.728341 |
| 1599 | 0.003183 | 0.027135 | 0.584021 |
| 1600 | 0.003303 | 0.02835 | 0.463076 |
| 1601 | 0.003179 | 0.02638 | 0.376498 |
| 1602 | 0.004066 | 0.027302 | 0.787392 |
| 1603 | 0.003293 | 0.027454 | 0.565816 |
| 1604 | 0.002846 | 0.02701 | 0.748421 |
| 1605 | 0.005887 | 0.032685 | 0.622649 |
| 1606 | 0.002889 | 0.02704 | 0.56754 |
| 1607 | 0.002831 | 0.026806 | 0.743947 |
| 1608 | 0.002965 | 0.027359 | 0.760013 |
| 1609 | 0.00396 | 0.027561 | 0.674454 |
| 1611 | 0.008586 | 0.075287 | 0.257982 |
| 1612 | 0.009539 | 0.076797 | 0.197109 |
| 1613 | 0.008018 | 0.072467 | 0.231769 |
| 1614 | 0.012023 | 0.075871 | 0.172221 |
| 1615 | 0.008328 | 0.071072 | 0.220705 |
| 1616 | 0.012023 | 0.075453 | 0.25357 |
| 1617 | 0.012384 | 0.075749 | 0.189438 |
| 1618 | 0.009907 | 0.074134 | 0.228244 |
| 1621 | 0.007789 | 0.070227 | 0.245667 |
| 1622 | 0.008908 | 0.072407 | 0.245921 |
| 1623 | 0.011175 | 0.077951 | 0.188074 |
| 1624 | 0.008036 | 0.069872 | 0.246323 |
| 1625 | 0.011073 | 0.074178 | 0.233123 |
| 1626 | 0.012397 | 0.076047 | 0.205037 |
| 1627 | 0.01196 | 0.082003 | 0.236073 |
| 1628 | 0.008087 | 0.073661 | 0.162851 |
| 1629 | 0.009228 | 0.071361 | 0.1983 |
| 1630 | 0.00787 | 0.068021 | 0.252184 |
| 1632 | 0.008463 | 0.070379 | 0.220109 |
| 1633 | 0.008069 | 0.070751 | 0.186138 |
| 1634 | 0.010343 | 0.072986 | 0.210934 |
| 1635 | 0.009638 | 0.077485 | 0.263883 |
| 1636 | 0.008719 | 0.074615 | 0.259029 |
| 1637 | 0.008512 | 0.073437 | 0.167086 |
| 1638 | 0.007929 | 0.072165 | 0.163977 |
| 1639 | 0.007868 | 0.07025 | 0.216269 |
| 1640 | 0.010217 | 0.075129 | 0.212716 |
| 1641 | 0.009846 | 0.072375 | 0.229234 |
| 1642 | 0.008044 | 0.072899 | 0.146821 |
| 1643 | 0.007903 | 0.073916 | 0.202451 |
| 1644 | 0.007997 | 0.072045 | 0.2269 |
| 1645 | 0.008005 | 0.071968 | 0.225724 |
| 1646 | 0.007868 | 0.072947 | 0.218694 |
| 1647 | 0.007874 | 0.07009 | 0.204347 |
| 1648 | 0.008126 | 0.072838 | 0.231684 |
| 1649 | 0.007985 | 0.07098 | 0.217341 |
| 1650 | 0.00805 | 0.073491 | 0.157452 |
| 1651 | 0.008583 | 0.074894 | 0.221222 |
| 1652 | 0.00782 | 0.073647 | 0.23366 |
| 1653 | 0.007727 | 0.072533 | 0.274822 |
| 1654 | 10.007898 | 0.072049 | 0.19207 |
| 1655 | 0.007809 | 0.072421 | 0.239521 |
| 1656 | 0.00814 | 0.070844 | 0.233632 |
| 1657 | 0.008044 | 0.069952 | 0.173161 |
| 1658 | 0.008147 | 0.070865 | 0.114916 |
| 1659 | 0.008054 | 0.070011 | 0.234503 |
| 1660 | 0.008051 | 0.073257 | 0.209313 |
| 1661 | 0.008006 | 0.073666 | 0.220585 |
| 1662 | 0.007882 | 0.072722 | 0.194041 |
| 1663 | 0.0079 | 0.07183 | 0.209789 |
| 1664 | 0.008074 | 0.069676 | 0.185531 |
| 1665 | 0.00878 | 0.071074 | 0.21086 |
| 1666 | 0.007895 | 0.071838 | 0.244376 |
| 1668 | 0.008053 | 0.072861 | 0.253493 |
| 1669 | 0.007873 | 0.073223 | 0.198109 |
| 1670 | 0.007816 | 0.068945 | 0.206857 |
| 1671 | 0.007989 | 0.071814 | 0.303432 |
| 1672 | 0.008207 | 0.073024 | 0.222844 |
| 1673 | 0.008259 | 0.0722 | 0.255081 |
| 1674 | 0.008145 | 0.071765 | 0.197578 |
| 1675 | 0.008243 | 0.07316 | 0.202955 |
| 1676 | 0.008121 | 0.07247 | 0.197388 |
| 1677 | 0.008023 | 0.071901 | 0.24222 |
| 1678 | 0.008907 | 0.074766 | 0.187839 |
| 1679 | 0.007883 | 0.072372 | 0.198258 |
| 1680 | 0.008303 | 0.07202 | 0.20641 |
| 1681 | 0.008051 | 0.068996 | 0.172375 |
| 1682 | 0.008184 | 0.073282 | 0.285672 |
| 1684 | 0.008361 | 0.072729 | 0.170928 |
| 1685 | 0.008716 | 0.074393 | 0.222029 |
| 1686 | 0.008059 | 0.071129 | 0.196348 |
| 1687 | 0.007953 | 0.070337 | 0.21236 |
| 1688 | 0.008643 | 0.070881 | 0.227238 |
| 1689 | 0.007905 | 0.069917 | 0.19241 |
| 1690 | 0.008127 | 0.072187 | 0.219756 |
| 1691 | 0.007958 | 0.073297 | 0.230226 |
| 1692 | 0.00806 | 0.069949 | 0.25326 |
| 1693 | 0.008331 | 0.073454 | 0.232442 |
| 1694 | 0.00995 | 0.07389 | 0.245006 |
| 1695 | 0.00813 | 0.070796 | 0.208166 |
| 1696 | 0.00828 | 0.070973 | 0.196401 |
| 1697 | 0.008427 | 0.070372 | 0.211389 |
| 1698 | 0.010244 | 0.071351 | 0.23418 |
| 1699 | 0.008127 | 0.071392 | 0.186818 |
| 1700 | 0.011627 | 0.078038 | 0.216352 |
| 1701 | 0.008158 | 0.071674 | 0.235836 |
| 1702 | 0.008405 | 0.072728 | 0.214028 |
| 1703 | 0.00931 | 0.072866 | 0.24933 |
| 1704 | 0.00834 | 0.06923 | 0.178878 |
| 1705 | 0.00998 | 0.07348 | 0.262106 |
| 1707 | 0.004095 | 0.007786 | 0.905391 |
| 1708 | 0.00319 | 0.007636 | 0.918093 |
| 1710 | 0.003179 | 0.007381 | 0.810737 |
| 1711 | 0.003206 | 0.007316 | 0.948158 |
| 1712 | 0.003223 | 0.007633 | 0.992454 |
| 1713 | 0.003147 | 0.007825 | 1.018484 |
| 1714 | 0.003156 | 0.007799 | 1.011062 |
| 1715 | 0.003144 | 0.007846 | 1.03755 |
| 1716 | 0.003126 | 0.007534 | 0.995065 |
| 1717 | 0.003303 | 0.008123 | 0.95218 |
| 1718 | 0.003174 | 0.007598 | 0.739364 |
| 1720 | 0.003107 | 0.007531 | 0.963791 |
| 1721 | 0.003184 | 0.007393 | 0.642433 |
| 1722 | 0.003234 | 0.007679 | 1.087014 |
| 1723 | 0.003183 | 0.007625 | 0.746413 |
| 1725 | 0.003166 | 0.00732 | 0.983384 |
| 1726 | 0.003192 | 0.007532 | 0.92262 |
| 1727 | 0.003216 | 0.007484 | 0.718343 |
| 1728 | 0.003213 | 0.00725 | 0.952505 |
| 1729 | 0.003225 | 0.007421 | 0.911589 |
| 1730 | 0.003281 | 0.007498 | 0.995307 |
| 1731 | 0.003053 | 0.007481 | 0.950875 |
| 1732 | 0.00316 | 0.007479 | 0.757397 |
| 1733 | 0.003214 | 0.007451 | 0.940672 |
| 1734 | 0.003219 | 0.007427 | 0.893661 |
| 1735 | 0.003254 | 0.007528 | 0.836399 |
| 1737 | 0.003227 | 0.007374 | 0.911326 |
| 1738 | 0.003214 | 0.007477 | 1.002511 |
| 1739 | 0.00327 | 0.008719 | 0.956423 |
| 1740 | 0.003147 | 0.007789 | 1.00538 |
| 1741 | 0.003279 | 0.007904 | 0.975752 |
| 1742 | 0.003162 | 0.007423 | 1.22881 |
| 1743 | 0.003139 | 0.007692 | 0.743436 |
| 1744 | 0.003205 | 0.007379 | 0.772479 |
| 1745 | 0.003182 | 0.007429 | 1.079506 |
| 1747 | 0.003253 | 0.0075 | 0.816336 |
| 1748 | 0.003362 | 0.008459 | 0.998276 |
| 1749 | 0.003015 | 0.007458 | 0.936332 |
| 1750 | 0.003212 | 0.008153 | 0.859119 |
| 1752 | 0.003259 | 0.007776 | 0.978627 |
| 1753 | 0.00325 | 0.007387 | 0.786866 |
| 1754 | 0.003162 | 0.006997 | 1.003224 |
| 1755 | 0.003259 | 0.007394 | 0.925965 |
| 1756 | 0.00318 | 0.007571 | 1.096225 |
| 1758 | 0.003337 | 0.007882 | 0.985142 |
| 1759 | 0.003418 | 0.008082 | 0.953122 |
| 1760 | 0.003232 | 0.007565 | 0.769634 |
| 1761 | 0.003209 | 0.007716 | 0.826567 |
| 1762 | 0.003197 | 0.007352 | 0.743692 |
| 1763 | 0.003236 | 0.007511 | 1.040475 |
| 1764 | 0.003251 | 0.007572 | 1.383275 |
| 1765 | 0.003257 | 0.013774 | 0.819945 |
| 1766 | 0.003138 | 0.007452 | 0.976752 |
| 1767 | 0.003206 | 0.007674 | 0.936511 |
| 1768 | 0.003234 | 0.008193 | 0.717088 |
| 1769 | 0.003178 | 0.007449 | 0.913192 |
| 1771 | 0.003187 | 0.007352 | 0.941054 |
| 1772 | 0.005576 | 0.019795 | 0.767377 |
| 1773 | 0.003294 | 0.012787 | 0.583382 |
| 1774 | 0.003183 | 0.007692 | 0.809862 |
| 1775 | 0.003131 | 0.007429 | 1.000089 |
| 1776 | 0.003174 | 0.007465 | 0.908376 |
| 1777 | 0.003238 | 0.00771 | 1.13952 |
| 1778 | 0.003232 | 0.007417 | 0.854898 |
| 1779 | 0.003218 | 0.007322 | 0.736095 |
| 1780 | 0.003511 | 0.008457 | 0.949085 |
| 1781 | 0.003302 | 0.009751 | 0.834468 |
| 1782 | 0.003093 | 0.007604 | 0.813662 |
| 1783 | 0.003061 | 0.007604 | 0.894994 |
| 1784 | 0.003161 | 0.007326 | 0.70474 |
| 1785 | 0.003225 | 0.007357 | 0.699541 |
| 1786 | 0.003164 | 0.007261 | 0.794668 |
| 1787 | 0.00315 | 0.007418 | 0.964931 |
| 1788 | 0.003283 | 0.007843 | 0.946281 |
| 1789 | 0.003291 | 0.007593 | 0.936091 |
| 1790 | 0.003761 | 0.00877 | 0.886762 |
| 1791 | 0.003112 | 0.007642 | 0.918008 |
| 1792 | 0.003268 | 0.007666 | 0.703354 |
| 1793 | 0.003133 | 0.007455 | 0.842638 |
| 1794 | 0.003159 | 0.007349 | 0.728775 |
| 1795 | 0.003246 | 0.007273 | 0.745148 |
| 1796 | 0.003613 | 0.007904 | 0.628593 |
| 1797 | 0.00427 | 0.008685 | 0.659605 |
| 1798 | 0.003091 | 0.007893 | 0.885414 |
| 1799 | 0.003119 | 0.007443 | 1.016389 |
| 1801 | 0.005041 | 0.009766 | 0.788892 |
| 1802 | 0.003799 | 0.009741 | 0.970487 |
| 1803 | 0.003191 | 0.007455 | 0.811591 |
| 1804 | 0.003256 | 0.007526 | 0.980185 |
| 1806 | 0.007832 | 0.015298 | 0.668837 |
| 1808 | 0.002099 | 0.00239 | 0.713927 |
| 1809 | 0.003792 | 0.004621 | 0.798598 |
| 1810 | 0.002017 | 0.002054 | 0.778629 |
| 1811 | 0.001935 | 0.002012 | 0.827105 |
| 1812 | 0.001955 | 0.002041 | 0.652207 |
| 1813 | 0.002017 | 0.002107 | 1.07398 |
| 1814 | 0.001992 | 0.00212 | 0.938161 |
| 1815 | 0.002057 | 0.002093 | 0.672235 |
| 1816 | 0.002067 | 0.002143 | 0.813551 |
| 1817 | 0.001993 | 0.00201 | 0.666663 |
| 1818 | 0.001943 | 0.002032 | 1.177111 |
| 1819 | 0.002001 | 0.002046 | 0.840791 |
| 1820 | 0.00202 | 0.002025 | 0.821089 |
| 1821 | 0.010528 | 0.014897 | 1.05278 |
| 1822 | 0.001978 | 0.002002 | 0.847594 |
| 1823 | 0.002033 | 0.002129 | 0.923411 |
| 1824 | 0.001995 | 0.002089 | 0.931098 |
| 1825 | 0.001951 | 0.002044 | 0.72614 |
| 1826 | 0.001901 | 0.002026 | 0.969977 |
| 1827 | 0.002017 | 0.002057 | 1.124311 |
| 1828 | 0.002003 | 0.002006 | 0.621247 |
| 1829 | 0.002024 | 0.002243 | 0.826881 |
| 1830 | 0.001924 | 0.001957 | 0.830188 |
| 1831 | 0.002058 | 0.002061 | 1.08971 |
| 1832 | 0.002021 | 0.002227 | 0.99019 |
| 1833 | 0.001961 | 0.002 | 1.043679 |
| 1834 | 0.001997 | 0.002049 | 1.079505 |
| 1835 | 0.002924 | 0.036425 | 0.9315 |
| 1836 | 0.002051 | 0.002049 | 0.764385 |
| 1837 | 0.001987 | 0.002013 | 0.939205 |
| 1838 | 0.001986 | 0.002074 | 0.945167 |
| 1839 | 0.00206 | 0.002081 | 0.757964 |
| 1840 | 0.001961 | 0.002069 | 0.963668 |
| 1841 | 0.001918 | 0.001952 | 0.824621 |
| 1842 | 0.001947 | 0.00206 | 0.828898 |
| 1844 | 0.002009 | 0.002099 | 0.812678 |
| 1845 | 0.001913 | 0.002007 | 0.924997 |
| 1846 | 0.002008 | 0.002012 | 1.067461 |
| 1847 | 0.002012 | 0.002021 | 0.910096 |
| 1848 | 0.002014 | 0.002092 | 1.070281 |
| 1849 | 0.00198 | 0.002053 | 1.115724 |
| 1850 | 0.00196 | 0.002017 | 0.780307 |
| 1851 | 0.001999 | 0.002057 | 0.88249 |
| 1852 | 0.001974 | 0.002022 | 0.630003 |
| 1853 | 0.00197 | 0.001976 | 0.893397 |
| 1854 | 0.002051 | 0.002022 | 0.767922 |
| 1855 | 0.002001 | 0.002064 | 1.128736 |
| 1856 | 0.002104 | 0.002101 | 1.08652 |
| 1857 | 0.001954 | 0.002061 | 0.814235 |
| 1858 | 0.001944 | 0.002018 | 0.855267 |
| 1859 | 0.001923 | 0.002007 | 1.245303 |
| 1860 | 0.001933 | 0.002022 | 0.820898 |
| 1861 | 0.001984 | 0.001993 | 0.92614 |
| 1862 | 0.002019 | 0.002032 | 1.0456 |
| 1863 | 0.002124 | 0.002046 | 0.904399 |
| 1864 | 0.002036 | 0.002068 | 0.957025 |
| 1865 | 0.001969 | 0.002058 | 0.878785 |
| 1866 | 0.001941 | 0.002003 | 0.88721 |
| 1867 | 0.001968 | 0.002036 | 0.765442 |
| 1868 | 0.002326 | 0.002549 | 0.633458 |
| 1869 | 0.002028 | 0.002049 | 0.630945 |
| 1870 | 0.002011 | 0.002052 | 1.044067 |
| 1871 | 0.002039 | 0.002032 | 0.858321 |
| 1872 | 0.002073 | 0.002103 | 0.894575 |
| 1874 | 0.00194 | 0.002077 | 0.741909 |
| 1876 | 0.002012 | 0.002203 | 0.77563 |
| 1877 | 0.002166 | 0.00293 | 0.909265 |
| 1878 | 0.002203 | 0.003175 | 0.764486 |
| 1879 | 0.002076 | 0.002159 | 0.870865 |
| 1880 | 0.002123 | 0.00211 | 0.974287 |
| 1882 | 0.001971 | 0.00206 | 1.194054 |
| 1883 | 0.001965 | 0.00198 | 0.850391 |
| 1884 | 0.001983 | 0.00208 | 0.739432 |
| 1885 | 0.004432 | 0.006831 | 0.921387 |
| 1886 | 0.001964 | 0.002047 | 0.905673 |
| 1887 | 0.002128 | 0.002131 | 0.970315 |
| 1888 | 0.00216 | 0.002141 | 0.900712 |
| 1889 | 0.001985 | 0.00209 | 0.904929 |
| 1890 | 0.002027 | 0.002135 | 0.942252 |
| 1891 | 0.001943 | 0.002032 | 1.031348 |
| 1892 | 0.001968 | 0.002051 | 0.673121 |
| 1894 | 0.002064 | 0.002078 | 1.215602 |
| 1895 | 0.002657 | 0.002895 | 0.952559 |
| 1897 | 0.004208 | 0.004197 | 0.877243 |
| 1898 | 0.001943 | 0.002098 | 0.720743 |
| 1899 | 0.001911 | 0.002064 | 0.771716 |
| 1900 | 0.001992 | 0.002012 | 1.042259 |
| 1902 | 0.002111 | 0.002087 | 0.877153 |
| 1903 | 0.002066 | 0.002106 | 1.055068 |
| 1904 | 0.003184 | 0.003144 | 1.09739 |
| 1909 | 0.002055 | 0.003948 | 0.847157 |
| 1912 | 0.00224 | 0.004186 | 0.742941 |
| 1914 | 0.002728 | 0.006378 | 1.015755 |
| 1915 | 0.002104 | 0.003971 | 1.11464 |
| 1916 | 0.002013 | 0.00378 | 0.838864 |
| 1917 | 0.002152 | 0.003877 | 0.908892 |
| 1919 | 0.002163 | 0.003718 | 0.800267 |
| 1921 | 0.002207 | 0.003959 | 0.605339 |
| 1922 | 0.002089 | 0.004028 | 0.664389 |
| 1923 | 0.002035 | 0.003926 | 0.807267 |
| 1924 | 0.002045 | 0.003803 | 0.75616 |
| 1925 | 0.002024 | 0.003787 | 0.78207 |
| 1926 | 0.002081 | 0.004003 | 0.919381 |
| 1927 | 0.002087 | 0.0039 | 0.87094 |
| 1928 | 0.002169 | 0.003842 | 0.85592 |
| 1929 | 0.002196 | 0.003892 | 1.04674 |
| 1930 | 0.002059 | 0.004072 | 0.758699 |
| 1931 | 0.002018 | 0.003893 | 0.935624 |
| 1932 | 0.00212 | 0.003782 | 0.741426 |
| 1935 | 0.002149 | 0.003827 | 0.781431 |
| 1936 | 0.002114 | 0.003987 | 0.983047 |
| 1939 | 0.002105 | 0.003927 | 1.012539 |
| 1940 | 0.002102 | 0.003894 | 0.972949 |
| 1943 | 0.002208 | 0.003969 | 0.819485 |
| 1944 | 0.002129 | 0.003932 | 0.90291 |
| 1949 | 0.002939 | 0.028633 | 0.702177 |
| 1951 | 0.00213 | 0.003953 | 0.756432 |
| 1954 | 0.002084 | 0.003994 | 0.716999 |
| 1955 | 0.002082 | 0.003928 | 0.924112 |
| 1956 | 0.002151 | 0.004369 | 0.83182 |
| 1959 | 0.002066 | 0.003906 | 0.943132 |
| 1960 | 0.002082 | 0.003897 | 0.845987 |
| 1962 | 0.002068 | 0.004065 | 0.677381 |
| 1964 | 0.002309 | 0.005092 | 0.691221 |
| 1965 | 0.002145 | 0.004599 | 1.02379 |
| 1966 | 0.002096 | 0.003928 | 0.821807 |
| 1967 | 0.002135 | 0.004049 | 0.784332 |
| 1969 | 0.00258 | 0.004963 | 0.869906 |
| 1970 | 0.002402 | 0.005499 | 0.802557 |
| 1971 | 0.003555 | 0.00803 | 0.915366 |
| 1972 | 0.002123 | 0.004341 | 1.002436 |
| 1973 | 0.002093 | 0.004195 | 0.739287 |
| 1974 | 0.002264 | 0.004213 | 1.090771 |
| 1975 | 0.00213 | 0.004172 | 0.890328 |
| 1976 | 0.002854 | 0.00629 | 1.03609 |
| 1977 | 0.0022 | 0.003857 | 0.750837 |
| 1978 | 0.002184 | 0.006424 | 0.789674 |
| 1979 | 0.00218 | 0.004072 | 1.041609 |
| 1980 | 0.002089 | 0.003918 | 0.742203 |
| 1981 | 0.002022 | 0.00393 | 0.802623 |
| 1983 | 0.00211 | 0.00387 | 0.823111 |
| 1985 | 0.00238 | 0.005063 | 0.920921 |
| 1987 | 0.002096 | 0.003989 | 0.820149 |
| 1988 | 0.002196 | 0.004207 | 0.733845 |
| 1993 | 0.002629 | 0.004548 | 0.923992 |
| 1994 | 0.003546 | 0.013836 | 0.780026 |
| 1996 | 0.00246 | 0.004831 | 0.793669 |
| 1998 | 0.005897 | 0.010175 | 0.864774 |
| 1999 | 0.002493 | 0.004889 | 0.91103 |
| 2001 | 0.003035 | 0.009213 | 0.760895 |
| 2004 | 0.009008 | 0.051959 | 0.315777 |
| 2006 | 0.007683 | 0.047495 | 0.479879 |
| 2007 | 0.00815 | 0.046714 | 0.350549 |
| 2009 | 0.007773 | 0.047424 | 0.356192 |
| 2010 | 0.008014 | 0.048727 | 0.376383 |
| 2011 | 0.007678 | 0.050682 | 0.388038 |
| 2012 | 0.007658 | 0.048585 | 0.40471 |
| 2013 | 0.00776 | 0.046273 | 0.269896 |
| 2014 | 0.007967 | 0.048251 | 0.353403 |
| 2015 | 0.008137 | 0.046658 | 0.338498 |
| 2016 | 0.007733 | 0.04596 | 0.356733 |
| 2017 | 0.007571 | 0.045682 | 0.396185 |
| 2018 | 0.007836 | 0.047143 | 0.396857 |
| 2019 | 0.008556 | 0.049704 | 0.356688 |
| 2020 | 0.007647 | 0.047487 | 0.406455 |
| 2021 | 0.007659 | 0.046888 | 0.36344 |
| 2022 | 0.007575 | 0.046464 | 0.411014 |
| 2023 | 0.008 | 0.046656 | 0.392491 |
| 2024 | 0.007671 | 0.046704 | 0.343154 |
| 2026 | 0.008452 | 0.049376 | 0.408859 |
| 2027 | 0.007698 | 0.046912 | 0.328525 |
| 2028 | 0.007683 | 0.046616 | 0.339399 |
| 2029 | 0.007666 | 0.046757 | 0.297973 |
| 2030 | 0.007675 | 0.046508 | 0.435685 |
| 2031 | 0.008134 | 0.04519 | 0.411958 |
| 2032 | 0.007675 | 0.045703 | 0.307964 |
| 2033 | 0.007603 | 0.046911 | 0.368773 |
| 2034 | 0.007487 | 0.046125 | 0.42095 |
| 2035 | 0.007824 | 0.048172 | 0.297846 |
| 2036 | 0.007336 | 0.048153 | 0.42074 |
| 2037 | 0.008092 | 0.047712 | 0.309579 |
| 2038 | 0.007771 | 0.046713 | 0.372379 |
| 2039 | 0.008147 | 0.047807 | 0.46323 |
| 2041 | 0.007752 | 0.046575 | 0.519016 |
| 2042 | 0.007808 | 0.047418 | 0.427933 |
| 2043 | 0.007841 | 0.048971 | 0.487233 |
| 2044 | 0.007751 | 0.04791 | 0.502666 |
| 2045 | 0.007997 | 0.04765 | 0.425789 |
| 2046 | 0.007681 | 0.047159 | 0.387429 |
| 2047 | 0.008297 | 0.04738 | 0.434821 |
| 2048 | 0.00876 | 0.04945 | 0.444633 |
| 2049 | 0.007392 | 0.046997 | 0.359452 |
| 2050 | 0.008459 | 0.051214 | 0.257893 |
| 2051 | 0.007739 | 0.048823 | 0.379033 |
| 2052 | 0.007651 | 0.047708 | 0.301475 |
| 2055 | 0.008096 | 0.046365 | 0.286331 |
| 2056 | 0.007633 | 0.045501 | 0.321644 |
| 2057 | 0.007723 | 0.04781 | 0.286926 |
| 2058 | 0.007596 | 0.047326 | 0.35697 |
| 2059 | 0.007819 | 0.048191 | 0.328469 |
| 2060 | 0.007522 | 0.049484 | 0.346836 |
| 2062 | 0.007728 | 0.04874 | 0.437359 |
| 2064 | 0.007614 | 0.047857 | 0.346887 |
| 2065 | 0.007718 | 0.04734 | 0.320753 |
| 2066 | 0.007861 | 0.048273 | 0.33439 |
| 2067 | 0.007787 | 0.048374 | 0.360579 |
| 2068 | 0.007899 | 0.047923 | 0.438593 |
| 2069 | 0.007997 | 0.047566 | 0.294398 |
| 2070 | 0.007618 | 0.047015 | 0.368413 |
| 2071 | 0.008211 | 0.047051 | 0.398477 |
| 2072 | 0.007816 | 0.045952 | 0.291645 |
| 2073 | 0.00744 | 0.047558 | 0.416445 |
| 2074 | 0.007748 | 0.047678 | 0.31422 |
| 2075 | 0.007821 | 0.049202 | 0.410995 |
| 2076 | 0.007579 | 0.048504 | 0.35862 |
| 2077 | 0.007606 | 0.048341 | 0.336701 |
| 2078 | 0.007833 | 0.047621 | 0.430521 |
| 2079 | 0.008881 | 0.04708 | 0.367176 |
| 2080 | 0.007632 | 0.046431 | 0.401737 |
| 2081 | 0.007585 | 0.047886 | 0.328978 |
| 2082 | 0.00782 | 0.048005 | 0.42346 |
| 2084 | 0.007872 | 0.048572 | 0.374299 |
| 2085 | 0.007508 | 0.048884 | 0.363426 |
| 2086 | 0.007641 | 0.04778 | 0.455748 |
| 2087 | 0.007576 | 0.046705 | 0.394438 |
| 2089 | 0.007588 | 0.050455 | 0.512216 |
| 2091 | 0.007914 | 0.049272 | 0.348157 |
| 2092 | 0.008757 | 0.085812 | 0.387008 |
| 2093 | 0.007033 | 0.04869 | 0.543809 |
| 2094 | 0.007412 | 0.048193 | 0.462981 |
| 2095 | 0.007785 | 0.048394 | 0.378497 |
| 2096 | 0.00808 | 0.04793 | 0.422319 |
| 2097 | 0.007585 | 0.052485 | 0.339995 |
| 2098 | 0.007722 | 0.077927 | 0.360082 |
| 2101 | 0.003196 | 0.02074 | 0.711343 |
| 2102 | 0.003171 | 0.019613 | 0.932187 |
| 2103 | 0.003015 | 0.019345 | 0.66012 |
| 2105 | 0.003183 | 0.019557 | 0.922989 |
| 2106 | 0.00304 | 0.019249 | 0.610898 |
| 2107 | 0.003086 | 0.020481 | 0.726218 |
| 2108 | 0.003083 | 0.01979 | 0.699435 |
| 2109 | 0.003083 | 0.019528 | 0.595743 |
| 2110 | 0.003043 | 0.01985 | 0.811218 |
| 2111 | 0.003105 | 0.019319 | 0.486517 |
| 2112 | 0.003041 | 0.018937 | 0.51889 |
| 2113 | 0.00304 | 0.019069 | 0.468082 |
| 2114 | 0.003108 | 0.018998 | 0.482694 |
| 2115 | 0.00313 | 0.019507 | 0.554585 |
| 2116 | 0.003166 | 0.020002 | 0.7362 |
| 2118 | 0.003188 | 0.01935 | 0.596176 |
| 2119 | 0.003134 | 0.019993 | 0.637627 |
| 2120 | 0.003081 | 0.019437 | 0.678214 |
| 2121 | 0.003146 | 0.019336 | 0.60839 |
| 2122 | 0.003065 | 0.019235 | 0.499429 |
| 2124 | 0.003203 | 0.019847 | 0.557828 |
| 2125 | 0.003239 | 0.02001 | 0.552858 |
| 2126 | 0.003195 | 0.019259 | 0.656 |
| 2127 | 0.003154 | 0.019186 | 0.483088 |
| 2129 | 0.003119 | 0.019051 | 0.620213 |
| 2130 | 0.003123 | 0.019615 | 0.424395 |
| 2134 | 0.003217 | 0.019331 | 0.536501 |
| 2135 | 0.003065 | 0.019226 | 0.52152 |
| 2137 | 0.003178 | 0.018652 | 0.698231 |
| 2138 | 0.003168 | 0.019197 | 0.587975 |
| 2140 | 0.003216 | 0.019528 | 0.625389 |
| 2141 | 0.00316 | 0.019709 | 0.682383 |
| 2142 | 0.003241 | 0.019746 | 0.516585 |
| 2143 | 0.00322 | 0.019625 | 0.613552 |
| 2144 | 0.003175 | 0.018746 | 0.700146 |
| 2145 | 0.003151 | 0.019352 | 0.390091 |
| 2146 | 0.003246 | 0.019676 | 0.504855 |
| 2147 | 0.00315 | 0.018806 | 0.691577 |
| 2148 | 0.00327 | 0.020065 | 0.647184 |
| 2149 | 0.003318 | 0.019742 | 0.593045 |
| 2150 | 0.003295 | 0.019112 | 0.545147 |
| 2151 | 0.003222 | 0.019324 | 0.623487 |
| 2153 | 0.003163 | 0.019031 | 0.622529 |
| 2154 | 0.003097 | 0.019212 | 0.412396 |
| 2155 | 0.003183 | 0.018864 | 0.628595 |
| 2157 | 0.003208 | 0.019445 | 0.512846 |
| 2158 | 0.00328 | 0.020175 | 0.608198 |
| 2159 | 0.00605 | 0.027206 | 0.523008 |
| 2160 | 0.003294 | 0.019204 | 0.60676 |
| 2161 | 0.003175 | 0.019186 | 0.58493 |
| 2162 | 0.003073 | 0.018774 | 0.676172 |
| 2163 | 0.003213 | 0.026017 | 0.717037 |
| 2164 | 0.003146 | 0.019228 | 0.629069 |
| 2165 | 0.003177 | 0.019712 | 0.651398 |
| 2166 | 0.015226 | 0.067595 | 0.77104 |
| 2168 | 0.003259 | 0.019199 | 0.709717 |
| 2169 | 0.003096 | 0.019088 | 0.552215 |
| 2170 | 0.003193 | 0.019073 | 0.843088 |
| 2171 | 0.003203 | 0.018715 | 0.663203 |
| 2173 | 0.003078 | 0.018872 | 0.596725 |
| 2174 | 0.003215 | 0.019295 | 0.586757 |
| 2175 | 0.003355 | 0.020379 | 0.591717 |
| 2176 | 0.00331 | 0.020075 | 0.621581 |
| 2178 | 0.003288 | 0.019762 | 0.563754 |
| 2179 | 0.023311 | 0.108889 | 0.634632 |
| 2180 | 0.003154 | 0.018821 | 0.582331 |
| 2181 | 0.003174 | 0.019114 | 0.467664 |
| 2182 | 0.003295 | 0.019446 | 0.780908 |
| 2183 | 0.003253 | 0.01977 | 0.567795 |
| 2184 | 0.003242 | 0.020002 | 0.791142 |
| 2185 | 0.003168 | 0.019575 | 0.775637 |
| 2186 | 0.003096 | 0.018948 | 0.568767 |
| 2187 | 0.003259 | 0.019195 | 0.523207 |
| 2188 | 0.003153 | 0.019022 | 0.565014 |
| 2189 | 0.003154 | 0.019989 | 0.535538 |
| 2190 | 0.003524 | 0.019646 | 0.64781 |
| 2191 | 0.004391 | 0.020707 | 0.675903 |
| 2192 | 0.00296 | 0.020679 | 0.695385 |
| 2193 | 0.00312 | 0.019868 | 0.56865 |
| 2194 | 0.003186 | 0.020148 | 0.67523 |
| 2195 | 0.003131 | 0.019043 | 0.529032 |
| 2196 | 0.003166 | 0.020125 | 0.702299 |
| 2197 | 0.003391 | 0.019802 | 0.716997 |
| 2198 | 0.007135 | 0.025543 | 0.647545 |
| 2200 | 0.006172 | 0.047972 | 0.218144 |
| 2201 | 0.006242 | 0.04465 | 0.132477 |
| 2202 | 0.00613 | 0.045688 | 0.181752 |
| 2203 | 0.006068 | 0.046964 | 0.152828 |
| 2204 | 0.005921 | 0.046084 | 0.14141 |
| 2205 | 0.006157 | 0.046238 | 0.191263 |
| 2206 | 0.006136 | 0.046016 | 0.190921 |
| 2207 | 0.005964 | 0.047186 | 0.153246 |
| 2208 | 0.006177 | 0.047117 | 0.155879 |
| 2210 | 0.006352 | 0.043635 | 0.146858 |
| 2211 | 0.006108 | 0.044772 | 0.155641 |
| 2212 | 0.006024 | 0.045543 | 0.142517 |
| 2213 | 0.005939 | 0.046344 | 0.143164 |
| 2214 | 0.006222 | 0.04614 | 0.192484 |
| 2215 | 0.006083 | 0.047678 | 0.16743 |
| 2216 | 0.006163 | 0.053998 | 0.13437 |
| 2217 | 0.006135 | 0.045467 | 0.137485 |
| 2218 | 0.006112 | 0.046402 | 0.156384 |
| 2219 | 0.006181 | 0.046612 | 0.162334 |
| 2220 | 0.006096 | 0.050543 | 0.162733 |
| 2221 | 0.006186 | 0.047334 | 0.152158 |
| 2222 | 0.006075 | 0.047502 | 0.194765 |
| 2223 | 0.006029 | 0.046467 | 0.137691 |
| 2224 | 0.006314 | 0.055065 | 0.20807 |
| 2226 | 0.006294 | 0.045913 | 0.209723 |
| 2227 | 0.006684 | 0.046979 | 0.190792 |
| 2228 | 0.006095 | 0.04607 | 0.165137 |
| 2229 | 0.006403 | 0.052747 | 0.16783 |
| 2231 | 0.006318 | 0.047325 | 0.120074 |
| 2232 | 0.006136 | 0.047645 | 0.154048 |
| 2233 | 0.008079 | 0.057783 | 0.168608 |
| 2234 | 0.006343 | 0.048289 | 0.130913 |
| 2238 | 0.006235 | 0.046307 | 0.168547 |
| 2239 | 0.006246 | 0.046488 | 0.140553 |
| 2241 | 0.006379 | 0.054148 | 0.24124 |
| 2242 | 0.006455 | 0.047961 | 0.131922 |
| 2243 | 0.006337 | 0.046456 | 0.186051 |
| 2244 | 0.006203 | 0.045602 | 0.125512 |
| 2245 | 0.006499 | 0.057369 | 0.154019 |
| 2246 | 0.006248 | 0.056398 | 0.149261 |
| 2247 | 0.006307 | 0.046397 | 0.228139 |
| 2248 | 0.006168 | 0.047874 | 0.112137 |
| 2249 | 0.006196 | 0.046805 | 0.155686 |
| 2250 | 0.00895 | 0.054401 | 0.183022 |
| 2251 | 0.00639 | 0.045554 | 0.169424 |
| 2252 | 0.006125 | 0.046755 | 0.150411 |
| 2253 | 0.00622 | 0.053633 | 0.191924 |
| 2254 | 0.006108 | 0.046631 | 0.13209 |
| 2255 | 0.00633 | 0.04723 | 0.135853 |
| 2256 | 0.006144 | 0.061456 | 0.112837 |
| 2257 | 0.006404 | 0.047664 | 0.179404 |
| 2258 | 0.00629 | 0.04559 | 0.115119 |
| 2259 | 0.006517 | 0.047495 | 0.139756 |
| 2260 | 0.006178 | 0.046241 | 0.166521 |
| 2261 | 0.006256 | 0.047309 | 0.21795 |
| 2262 | 0.006307 | 0.046961 | 0.155011 |
| 2263 | 0.006309 | 0.046877 | 0.215311 |
| 2264 | 0.00632 | 0.047891 | 0.153035 |
| 2265 | 0.006283 | 0.058641 | 0.211202 |
| 2266 | 0.006381 | 0.047328 | 0.134896 |
| 2267 | 0.006567 | 0.045783 | 0.135634 |
| 2269 | 0.006447 | 0.046908 | 0.133405 |
| 2270 | 0.006417 | 0.04824 | 0.139725 |
| 2271 | 0.00618 | 0.046553 | 0.166047 |
| 2272 | 0.006225 | 0.047393 | 0.160067 |
| 2273 | 0.006276 | 0.047685 | 0.170297 |
| 2274 | 0.00624 | 0.04688 | 0.198238 |
| 2275 | 0.006534 | 0.046315 | 0.157348 |
| 2276 | 0.007069 | 0.047997 | 0.152782 |
| 2277 | 0.009026 | 0.05061 | 0.196609 |
| 2278 | 0.014832 | 0.067865 | 0.14616 |
| 2279 | 0.006428 | 0.046196 | 0.131052 |
| 2280 | 0.006224 | 0.046989 | 0.145311 |
| 2281 | 0.006154 | 0.047328 | 0.14968 |
| 2282 | 0.006248 | 0.046031 | 0.150055 |
| 2283 | 0.006174 | 0.047343 | 0.163964 |
| 2284 | 0.008235 | 0.4302 | 0.20482 |
| 2285 | 1.795726 | 2.705619 | 0.273225 |
| 2286 | 0.008775 | 0.050342 | 0.1587 |
| 2287 | 0.00631 | 0.047599 | 0.134257 |
| 2288 | 0.006544 | 0.046959 | 0.161388 |
| 2289 | 0.008183 | 0.050593 | 0.141202 |
| 2290 | 0.006064 | 0.048815 | 0.175503 |
| 2291 | 0.00618 | 0.046271 | 0.152712 |
| 2292 | 0.007016 | 0.048265 | 0.181027 |
| 2293 | 0.008176 | 0.049316 | 0.164933 |
| 2294 | 0.006486 | 0.048746 | 0.152462 |
| 2295 | 0.006359 | 0.047944 | 0.131901 |
| 2296 | 0.00722 | 0.04902 | 0.134938 |
| 2299 | 0.001841 | 0.021836 | 0.688111 |
| 2300 | 0.086641 | 0.166328 | 0.690494 |
| 2301 | 0.001849 | 0.020911 | 0.564046 |
| 2302 | 0.001837 | 0.020036 | 0.674931 |
| 2303 | 0.001843 | 0.020641 | 0.808588 |
| 2304 | 0.001836 | 0.020295 | 0.58194 |
| 2305 | 0.001822 | 0.020215 | 0.654307 |
| 2306 | 0.002438 | 0.022581 | 0.736907 |
| 2307 | 0.00178 | 0.021066 | 0.620358 |
| 2308 | 0.001832 | 0.020095 | 0.668554 |
| 2309 | 0.00178 | 0.019898 | 0.763666 |
| 2310 | 0.003472 | 0.020626 | 0.667713 |
| 2311 | 0.001796 | 0.019725 | 0.588065 |
| 2312 | 0.001799 | 0.020161 | 0.680887 |
| 2313 | 0.001785 | 0.020109 | 0.751756 |
| 2314 | 0.001785 | 0.020767 | 0.734117 |
| 2315 | 0.001731 | 0.021027 | 0.575685 |
| 2316 | 0.001777 | 0.020545 | 0.621465 |
| 2317 | 0.001726 | 0.020462 | 0.635235 |
| 2319 | 0.001783 | 0.019558 | 0.623992 |
| 2320 | 0.001783 | 0.019848 | 0.456169 |
| 2321 | 0.001819 | 0.019947 | 0.557319 |
| 2322 | 0.001816 | 0.020314 | 0.849308 |
| 2323 | 0.001812 | 0.021185 | 0.5437 |
| 2324 | 0.002117 | 0.021167 | 0.623834 |
| 2325 | 0.001774 | 0.020086 | 0.705615 |
| 2326 | 0.001762 | 0.020397 | 0.609166 |
| 2327 | 0.001838 | 0.019555 | 0.516895 |
| 2328 | 0.001819 | 0.019407 | 0.566088 |
| 2329 | 0.001846 | 0.020255 | 0.675583 |
| 2330 | 0.001822 | 0.020372 | 0.658319 |
| 2331 | 0.00183 | 0.020907 | 0.712729 |
| 2332 | 0.001716 | 0.020645 | 0.633101 |
| 2333 | 0.001761 | 0.020053 | 0.496843 |
| 2334 | 0.001795 | 0.020116 | 0.470328 |
| 2335 | 0.001973 | 0.019994 | 0.69908 |
| 2336 | 0.002355 | 0.021316 | 0.639826 |
| 2337 | 0.002071 | 0.019995 | 0.717695 |
| 2338 | 0.003361 | 0.022952 | 0.608106 |
| 2339 | 0.002512 | 0.022295 | 0.934816 |
| 2340 | 0.001872 | 0.02124 | 0.653365 |
| 2341 | 0.001758 | 0.020188 | 0.628362 |
| 2342 | 0.00179 | 0.020031 | 0.430364 |
| 2343 | 0.00225 | 0.021727 | 0.620708 |
| 2344 | 0.487092 | 1.242389 | 0.705218 |
| 2346 | 0.62511 | 0.991799 | 0.755326 |
| 2347 | 0.007601 | 0.031311 | 0.516649 |
| 2348 | 0.003801 | 0.024391 | 0.44802 |
| 2349 | 0.001763 | 0.020418 | 0.710152 |
| 2352 | 0.001967 | 0.020354 | 0.674644 |
| 2353 | 0.002343 | 0.020885 | 0.451139 |
| 2354 | 0.005892 | 0.03064 | 0.7194 |
| 2355 | 0.002628 | 0.021243 | 0.487594 |
| 2356 | 0.002016 | 0.021037 | 0.615679 |
| 2357 | 0.002143 | 0.021701 | 0.386956 |
| 2358 | 0.00212 | 0.021198 | 0.829166 |
| 2359 | 0.001791 | 0.020172 | 0.544619 |
| 2360 | 0.001856 | 0.020163 | 0.486583 |
| 2361 | 0.001901 | 0.019958 | 0.530139 |
| 2362 | 0.001879 | 0.020308 | 0.653006 |
| 2363 | 0.001922 | 0.020067 | 0.590144 |
| 2364 | 0.001837 | 0.02092 | 0.592571 |
| 2365 | 0.001792 | 0.020929 | 0.551517 |
| 2366 | 0.001791 | 0.020276 | 0.562391 |
| 2367 | 0.001762 | 0.020237 | 0.490932 |
| 2368 | 0.001774 | 0.019958 | 0.581639 |
| 2369 | 0.002441 | 0.023886 | 0.442913 |
| 2370 | 0.001851 | 0.019744 | 0.597586 |
| 2371 | 0.001833 | 0.019874 | 0.780994 |
| 2372 | 0.001836 | 0.020828 | 0.624654 |
| 2373 | 0.002203 | 0.0221 | 0.648313 |
| 2374 | 0.001781 | 0.02029 | 0.477161 |
| 2375 | 0.001758 | 0.019773 | 0.487119 |
| 2376 | 0.001766 | 0.019771 | 0.526721 |
| 2377 | 0.001848 | 0.019724 | 0.617253 |
| 2379 | 0.001845 | 0.020022 | 0.490031 |
| 2380 | 0.001881 | 0.020903 | 0.584369 |
| 2381 | 0.329765 | 0.386006 | 0.504277 |
| 2382 | 0.002171 | 0.020794 | 0.601362 |
| 2383 | 0.001822 | 0.020056 | 0.529306 |
| 2384 | 0.001781 | 0.019899 | 0.518998 |
| 2385 | 0.002162 | 0.020271 | 0.525008 |
| 2386 | 0.001834 | 0.019886 | 0.548855 |
| 2387 | 0.002131 | 0.020496 | 0.494099 |
| 2388 | 0.002937 | 0.022063 | 0.622276 |
| 2390 | 0.001843 | 0.021049 | 0.592485 |
| 2392 | 0.001773 | 0.020597 | 0.495091 |
| 2394 | 0.001773 | 0.020237 | 0.601764 |
| 2395 | 0.001806 | 0.020086 | 0.558798 |
| 2396 | 0.001807 | 0.02021 | 0.456341 |
| 2397 | 0.002983 | 0.022137 | 0.664263 |
| 2399 | 0.002032 | 0.028561 | 0.419347 |
| 2400 | 0.001953 | 0.02732 | 0.539873 |
| 2401 | 0.001912 | 0.027274 | 0.638418 |
| 2402 | 0.001914 | 0.026898 | 0.523509 |
| 2403 | 0.001954 | 0.027389 | 0.678695 |
| 2404 | 0.001977 | 0.027124 | 0.765805 |
| 2405 | 0.00211 | 0.030541 | 0.701647 |
| 2406 | 0.00247 | 0.029078 | 0.813564 |
| 2407 | 0.002076 | 0.027414 | 0.698683 |
| 2408 | 0.001925 | 0.028973 | 0.656613 |
| 2409 | 0.002188 | 0.027465 | 0.541032 |
| 2410 | 0.001926 | 0.025909 | 0.545951 |
| 2411 | 0.00194 | 0.026781 | 0.754864 |
| 2412 | 0.00382 | 0.039591 | 0.762652 |
| 2413 | 0.002162 | 0.027084 | 0.685745 |
| 2414 | 0.002494 | 0.029168 | 0.594618 |
| 2415 | 0.002268 | 0.029022 | 0.768169 |
| 2416 | 0.001922 | 0.026555 | 0.470209 |
| 2417 | 0.002127 | 0.027122 | 0.636734 |
| 2418 | 0.001951 | 0.026994 | 0.650385 |
| 2419 | 0.001945 | 0.027143 | 0.883213 |
| 2420 | 0.002209 | 0.02794 | 0.572425 |
| 2421 | 0.003578 | 0.031521 | 0.65348 |
| 2422 | 0.4597 | 1.118984 | 0.742345 |
| 2423 | 0.002036 | 0.027481 | 0.594989 |
| 2424 | 0.001912 | 0.026795 | 0.566853 |
| 2425 | 0.002128 | 0.027336 | 0.616199 |
| 2426 | 0.004274 | 0.035029 | 0.701308 |
| 2427 | 0.002039 | 0.026637 | 0.688343 |
| 2428 | 0.002025 | 0.026834 | 0.482521 |
| 2430 | 0.002201 | 0.027234 | 0.454862 |
| 2431 | 0.002645 | 0.028863 | 0.606143 |
| 2432 | 0.002137 | 0.027883 | 0.73667 |
| 2433 | 0.001911 | 0.026777 | 0.764379 |
| 2434 | 0.002656 | 0.031144 | 0.654025 |
| 2435 | 0.357958 | 1.054187 | 0.789014 |
| 2436 | 0.002408 | 0.027834 | 0.652141 |
| 2437 | 0.002128 | 0.029482 | 0.589591 |
| 2438 | 0.002046 | 0.027328 | 0.6305 |
| 2439 | 0.002126 | 0.027527 | 0.702897 |
| 2441 | 0.001931 | 0.027353 | 0.554509 |
| 2442 | 0.00194 | 0.026835 | 0.748374 |
| 2443 | 0.00206 | 0.027504 | 0.580563 |
| 2444 | 0.002454 | 0.030028 | 0.562538 |
| 2445 | 0.002479 | 0.027979 | 0.691218 |
| 2446 | 0.002005 | 0.026464 | 0.592756 |
| 2447 | 0.00209 | 0.02736 | 0.67768 |
| 2448 | 0.00215 | 0.027489 | 0.746934 |
| 2449 | 0.001919 | 0.027314 | 0.63539 |
| 2450 | 0.001947 | 0.026788 | 0.971937 |
| 2451 | 0.002047 | 0.027081 | 0.714018 |
| 2452 | 0.00199 | 0.026765 | 0.687724 |
| 2453 | 0.002001 | 0.026884 | 0.675106 |
| 2454 | 0.002043 | 0.026814 | 0.578459 |
| 2455 | 0.002485 | 0.027898 | 0.62214 |
| 2456 | 0.003114 | 0.03194 | 0.536157 |
| 2457 | 0.002044 | 0.028064 | 0.672813 |
| 2458 | 0.015881 | 0.100316 | 0.627601 |
| 2460 | 0.00195 | 0.026811 | 0.61264 |
| 2461 | 0.001936 | 0.027321 | 0.614411 |
| 2462 | 0.001952 | 0.026879 | 0.570422 |
| 2463 | 0.001996 | 0.026846 | 0.724248 |
| 2464 | 0.003014 | 0.029551 | 0.681079 |
| 2465 | 0.851106 | 1.519622 | 0.556387 |
| 2466 | 0.001875 | 0.027585 | 0.573732 |
| 2467 | 0.001914 | 0.026865 | 0.584638 |
| 2468 | 0.001951 | 0.026667 | 0.76513 |
| 2469 | 0.001935 | 0.026646 | 0.483364 |
| 2470 | 0.001927 | 0.027128 | 0.708523 |
| 2471 | 0.002115 | 0.027369 | 0.645251 |
| 2472 | 0.002277 | 0.02739 | 0.83229 |
| 2473 | 0.003074 | 0.029112 | 0.868278 |
| 2474 | 0.001898 | 0.031517 | 0.628315 |
| 2475 | 0.001888 | 0.025737 | 0.488711 |
| 2476 | 0.001895 | 0.026321 | 0.539154 |
| 2477 | 0.001942 | 0.02957 | 0.540224 |
| 2478 | 0.001972 | 0.026698 | 0.560129 |
| 2479 | 0.002055 | 0.026361 | 0.588692 |
| 2480 | 0.002449 | 0.029257 | 0.564031 |
| 2481 | 0.002188 | 0.027339 | 0.617131 |
| 2482 | 0.001929 | 0.027532 | 0.647844 |
| 2483 | 0.001901 | 0.026611 | 0.505461 |
| 2484 | 0.001911 | 0.026331 | 0.632902 |
| 2485 | 0.001935 | 0.026733 | 0.781338 |
| 2486 | 0.00232 | 0.028127 | 0.762296 |
| 2487 | 0.002045 | 0.029716 | 0.555858 |
| 2488 | 0.002322 | 0.027239 | 0.655834 |
| 2489 | 0.003042 | 0.02839 | 0.619313 |
| 2490 | 0.001901 | 0.027794 | 0.675581 |
| 2491 | 0.001867 | 0.026896 | 0.63697 |
| 2492 | 0.001957 | 0.027113 | 0.720652 |
| 2493 | 0.00211 | 0.02692 | 0.928346 |
| 2494 | 0.001976 | 0.02681 | 0.505157 |
| 2496 | 0.005512 | 0.03457 | 0.661025 |
| 2498 | 0.001855 | 0.021094 | 1.26703 |
| 2499 | 0.001794 | 0.019382 | 1.205979 |
| 2500 | 0.001798 | 0.019209 | 1.194211 |
| 2501 | 0.001855 | 0.019647 | 1.175705 |
| 2502 | 0.001805 | 0.01951 | 1.05585 |
| 2503 | 0.001854 | 0.01969 | 0.740211 |
| 2504 | 0.001802 | 0.019108 | 1.169167 |
| 2505 | 0.001803 | 0.020364 | 1.044078 |
| 2507 | 0.001816 | 0.019238 | 0.826173 |
| 2508 | 0.0018 | 0.019457 | 0.747832 |
| 2509 | 0.001814 | 0.018725 | 0.764182 |
| 2510 | 0.001788 | 0.018705 | 0.961773 |
| 2511 | 0.001811 | 0.019187 | 1.002978 |
| 2512 | 0.00184 | 0.019337 | 0.951505 |
| 2513 | 0.001795 | 0.0198 | 0.784969 |
| 2514 | 0.001806 | 0.019423 | 1.155331 |
| 2515 | 0.001819 | 0.019277 | 0.899568 |
| 2516 | 0.001783 | 0.018727 | 0.906725 |
| 2517 | 0.001767 | 0.018491 | 1.156806 |
| 2518 | 0.001826 | 0.018843 | 0.984126 |
| 2519 | 0.001827 | 0.018971 | 1.106655 |
| 2520 | 0.001856 | 0.019129 | 0.981249 |
| 2521 | 0.001863 | 0.019361 | 0.815735 |
| 2522 | 0.001753 | 0.019788 | 0.683346 |
| 2523 | 0.00179 | 0.018958 | 0.763397 |
| 2524 | 0.001774 | 0.019189 | 0.99147 |
| 2525 | 0.001796 | 0.019005 | 1.173316 |
| 2526 | 0.00239 | 0.022053 | 0.703461 |
| 2527 | 0.001839 | 0.018912 | 0.926152 |
| 2528 | 0.002235 | 0.020166 | 0.849943 |
| 2529 | 0.001833 | 0.019542 | 1.169762 |
| 2531 | 0.001757 | 0.019516 | 0.804991 |
| 2532 | 0.001791 | 0.019332 | 0.97138 |
| 2533 | 0.00183 | 0.018744 | 0.932083 |
| 2534 | 0.001829 | 0.018966 | 0.603137 |
| 2535 | 0.001825 | 0.019067 | 0.775401 |
| 2536 | 0.001839 | 0.019239 | 1.005791 |
| 2537 | 0.001837 | 0.019027 | 0.742716 |
| 2538 | 0.00184 | 0.019119 | 1.025351 |
| 2539 | 0.001786 | 0.019252 | 1.038877 |
| 2540 | 0.001818 | 0.019031 | 1.121716 |
| 2541 | 0.001778 | 0.018762 | 0.803414 |
| 2542 | 0.00183 | 0.01897 | 0.935541 |
| 2543 | 0.001856 | 0.018641 | 0.779233 |
| 2544 | 0.001832 | 0.018997 | 0.936523 |
| 2545 | 0.001818 | 0.019351 | 0.785605 |
| 2546 | 0.001855 | 0.019618 | 1.078452 |
| 2547 | 0.001753 | 0.019425 | 0.952274 |
| 2548 | 0.001801 | 0.019016 | 0.883621 |
| 2549 | 0.001932 | 0.019884 | 0.963362 |
| 2550 | 0.001852 | 0.019493 | 0.756811 |
| 2551 | 0.001811 | 0.018994 | 0.735339 |
| 2552 | 0.001829 | 0.018641 | 0.751335 |
| 2553 | 0.001842 | 0.019264 | 0.734602 |
| 2555 | 0.001803 | 0.019524 | 0.791215 |
| 2556 | 0.001808 | 0.019326 | 1.234156 |
| 2557 | 0.001817 | 0.019034 | 0.844118 |
| 2559 | 0.0018 | 0.019361 | 0.832718 |
| 2560 | 0.001849 | 0.018958 | 1.108257 |
| 2561 | 0.001875 | 0.019191 | 0.924327 |
| 2562 | 0.00183 | 0.019757 | 0.913942 |
| 2563 | 0.001766 | 0.019449 | 1.039711 |
| 2564 | 0.001761 | 0.019642 | 0.975257 |
| 2565 | 0.001822 | 0.018907 | 0.875283 |
| 2566 | 0.001827 | 0.018953 | 30.804406 |
| 2567 | 0.00182 | 0.01881 | 0.84509 |
| 2568 | 0.001815 | 0.018919 | 0.949194 |
| 2569 | 0.00187 | 0.018963 | 0.839079 |
| 2570 | 0.001859 | 0.019606 | 0.843373 |
| 2571 | 0.001771 | 0.019425 | 0.664874 |
| 2572 | 0.001861 | 0.019316 | 0.807564 |
| 2573 | 0.001825 | 0.018896 | 1.142032 |
| 2574 | 0.001799 | 0.018916 | 0.733867 |
| 2575 | 0.001848 | 0.018948 | 0.764302 |
| 2576 | 0.001862 | 0.0189 | 0.934422 |
| 2578 | 0.001938 | 0.019715 | 0.942217 |
| 2579 | 0.001762 | 0.019242 | 0.986229 |
| 2580 | 0.001771 | 0.01972 | 0.75995 |
| 2581 | 0.001846 | 0.019138 | 0.813534 |
| 2582 | 0.001812 | 0.018893 | 1.03767 |
| 2583 | 0.001829 | 0.019116 | 0.974132 |
| 2584 | 0.001881 | 0.019667 | 0.850391 |
| 2585 | 0.002575 | 0.020133 | 1.050868 |
| 2586 | 0.002919 | 0.020784 | 0.982573 |
| 2587 | 0.001816 | 0.019662 | 0.675518 |
| 2588 | 0.001816 | 0.019437 | 0.754413 |
| 2589 | 0.001844 | 0.01968 | 0.725135 |
| 2590 | 0.00185 | 0.020018 | 0.754455 |
| 2591 | 0.001831 | 0.019558 | 0.900126 |
| 2593 | 0.001894 | 0.019772 | 0.853028 |
| 2594 | 0.003467 | 0.021701 | 0.877 |

### Example 79

### Expression of CD19-41BBζ, CD19-CD28ζ, HER2-41BBζ or HER2-CD28ζ Chimeric Antigen Receptors (CARs) Following In Vitro Administration of Circular RNAs Encoding Said CARs

Human T cells from three unique T cells donors were activated with anti-CD3 or CD28 for three days and set to rest for 24 hours. Engineered circular RNA (oRNA) encoding CD19-41BBζ, CD19-CD28ζ, HER2-41BBζ, or HER2-CD28ζ chimeric antigen receptors (CARs) were electroporated into the human T cells. 24 hours post transfection CAR expression for each of the CARs were measured using fluorescence-activated cell sorting (FACS).

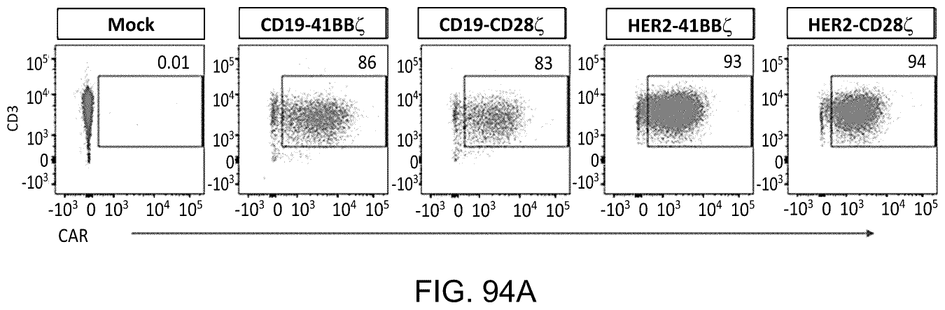

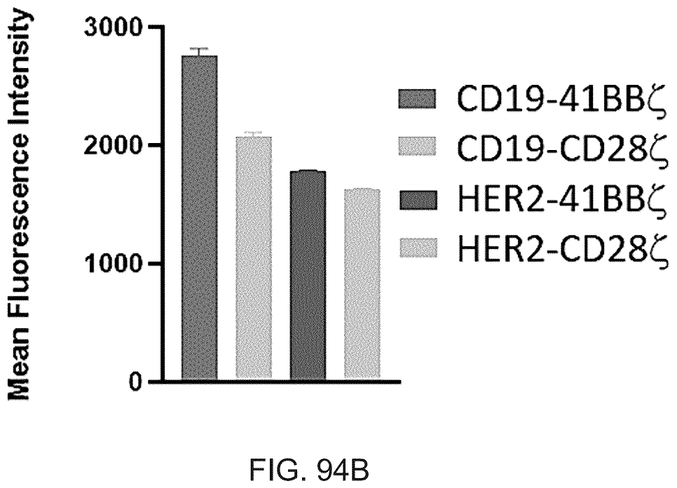

As seen in FIG. 94A and FIG. 94B, the circular RNA constructs were able to express CAR for each of the different CAR constructs.

### Example 80

### Lysis of Tumor Cells Following In Vitro Transfection of CD19-41BBζ and CD19-CD28ζ Chimeric Antigen Receptors (CARs) and Cytokine Production

Human T cells were activated with anti-CD3 or CD28 for three days and rested for 24 hours. The cells were then transfected with circular RNAs encoding for mOX40L (positive control), CD19-41BBζ, CD19-CD28ζ, HER2-41BBζ, or HER2-CD28ζ chimeric antigen receptors (CARs) via electroporation. 24 hours post-electroporation, said T cells were cocultured with Nalm6 tumor cells at a 1:1 effector to target (E:T) ratio. Nalm6 killing was measured 24 hours following coculturing of the human T cells with the Nalm6 tumor cells. Coculture supernatants were collect and analyzed using Meso Scale Discovery (MSD) (Agilent Technologies, Santa Clara, CA) for IFN-g and IL-2 expression.

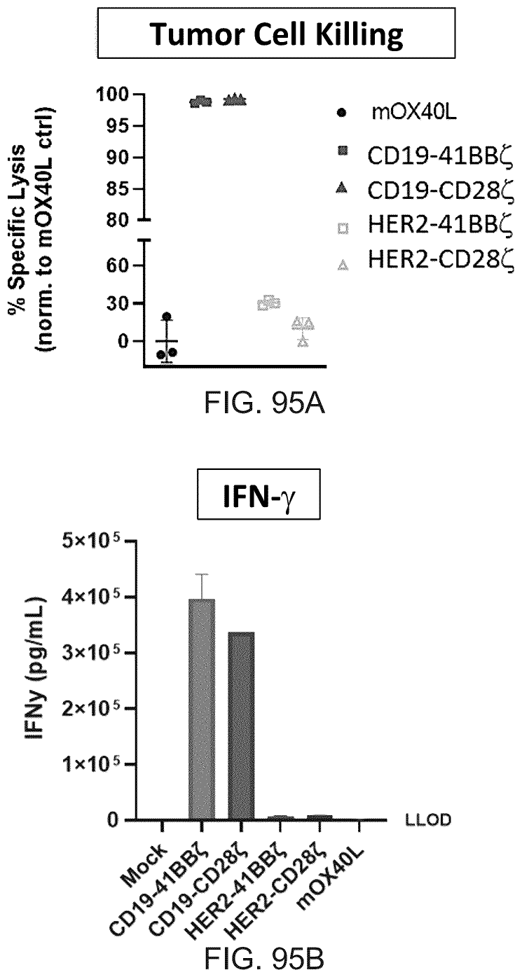

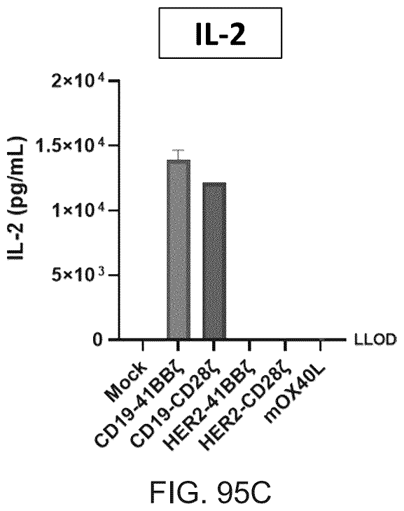

As seen in FIG. 95A, CD19-41BBζ and CD19-CD28ζ encoding circular RNAs surpassed tumor cell killing to the positive control, mOX40L circular RNAs, and even surpassed HER2-41BBζ and HER2-CD28ζ. The circRNAs encoding CD19-BBζ and CD19-28ζ also surpassed the IFN-g and IL-2 cytokine compared to the circRNAs encoding HER2-41BBζ, HER2-CD28ζ, or mOX40L (FIG. 95B and FIG. 95C). Antigen specific killing was observed at 1:3 effector to target (E:T) ratio following transfection of the circRNAs.

### Example 81

mOX40L Expression in Blood and Splenic Human T Cells of Humanized Mice Following Intravenous Administration of LNP-circRNA Encoding mOX40L

NOG mice (1E7 Mouse Hybridoma) were engrafted with human peripheral blood mononuclear cells (hPBMC). 3 weeks later the mice were treated with a single intravenous dose of LNP encapsulating circular RNAs encoding mOx40L at 2 mg/kg. LNP were formulated with ionizable lipid 10b-15, 10a-27, or 10a-26. 24 hours following intravenous administration, splenocytes and peripheral blood cells were isolated and analyzed for T cell expression of mOX40L.

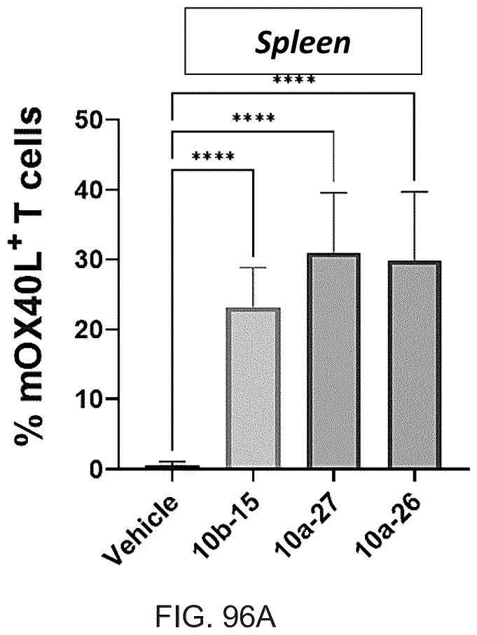

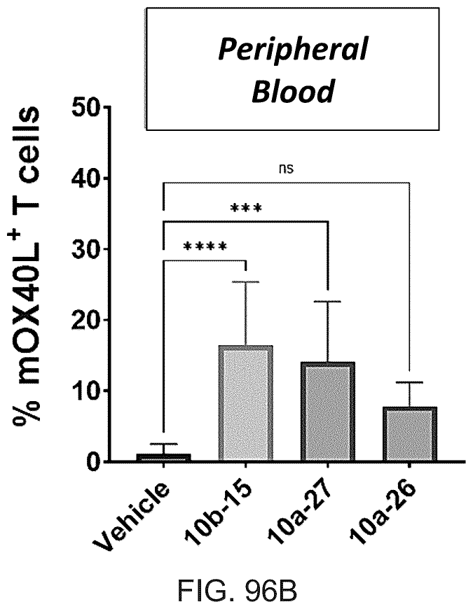

As seen in FIG. 96A and FIG. 96B, mOX40L was detected in the human T cells of the humanized mice's spleen and peripheral blood for each of the LNP constructs. Expression was higher for all three constructs in the spleen.

### Example 82

### B Cell Aplasia in the Spleen of Humanized Mice Following Intravenous Administration of LNP-circRNA Construct Encoding for an Anti-CD19 CAR

NOG mice (1E7 Mouse Hybridoma) were engrafted with human peripheral blood mononuclear cells (hPBMC) at 50-60% human engraftment in total. 3 weeks after engraftment, the mice were treated with a single dose of LNPs encapsulating circular RNA encoding anti-CD19 CAR or IXPBS (negative control) at 2 mg/kg. Six days post dosing, splenocytes were isolated and analyzed for the presence of B cells via CD19 expression.

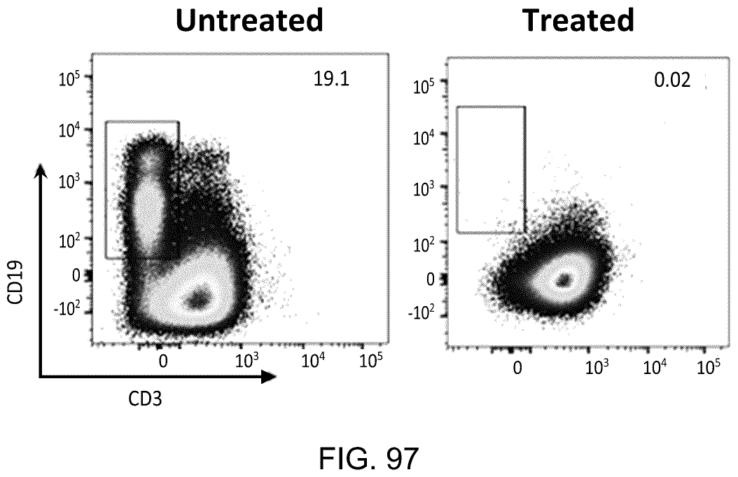

As seen in FIG. 97, B cell aplasia was present in the humanized mice when comparing the splenocytes of the mice receiving LNP-circRNA constructs (the left of FIG. 97) compared to the splenocytes of the mice receiving the negative control (the right of FIG. 97).

### Example 83

### Nalm6 Killing Following In Vitro Administration of Circular RNA Constructs Encoding Different CARs

T cells were activated with anti-CD3 or anti-CD28 for three days and rested for 24 hours. The cells were then treated with LNP-circRNAs encoding mOX40L (positive control), CD19-41BBζ, CD19-CD28ζ, HER2-41BBζ, or HER2-CD28ζ chimeric antigen receptors (CARs). LNPs were formulated using ionizable lipid 10b-15. Expression of the CARs was measured by fluorescence-activated cell sorting (FACS) to measure the mean fluorescence activity (MFI). T cells were then cocultured with Nalm6 tumor cells. Nalm6 killing was measured the following day.

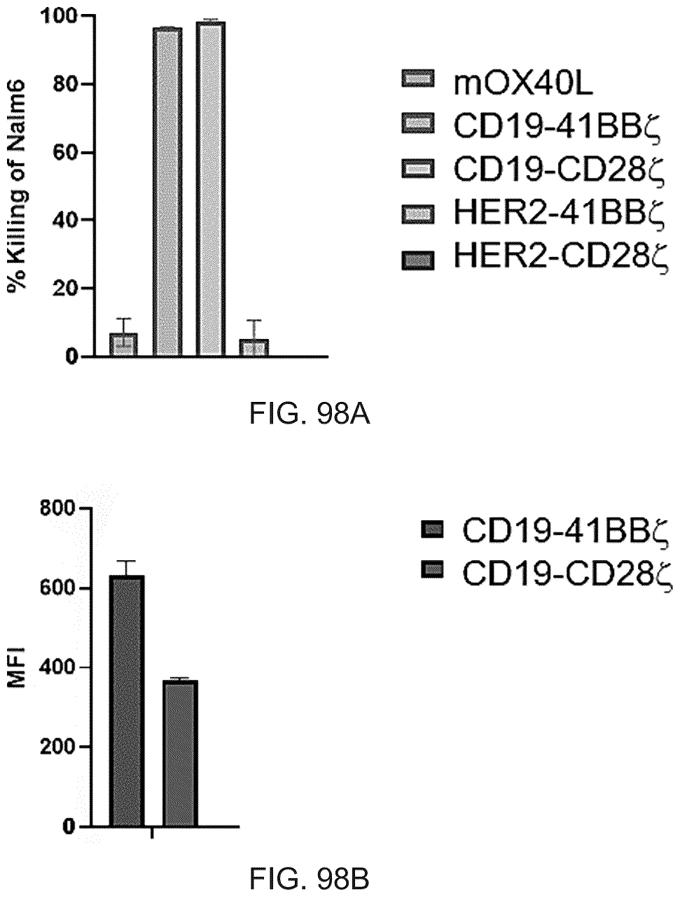

48 hours after co-culturing the T cells with the Nalm6 cells, the effector to target (E:T) ratio was 1:5. As seen in FIG. 98A, percent killing of the Nalm6 tumor cells post administration of the LNP-circRNA constructs was significantly greater for the constructs encoding CD19-CD28ζ or CD19-41BBζ compared to the constructs encoding HER2-41BBζ, HER2-CD28ζ, or mOX40L (the positive control). FIG. 98B illustrates that circRNAs encoding CD19-28ζ CARs has a greater killing of Nalm6 cells than circRNAs encoding CD19-BBζ CARs.

### Example 84

1E6 (Nalm6-Luc) cells were engrafted into NSG mice. 3 days later, CD3/CD28 treated hPBMCs were engrafted ex vivo. 24 hours post engraftment, the mice were dosed via in vivo administration with LNP encapsulating circular RNA constructs (circular RNAs encoded either CD19-41BBζ CARs, CD19-CD28ζCARs, HER2-41BBζ CARs, HER2-CD28ζCARs or mOX40L (positive control)) or PBS (negative control 1) or PBMC (negative control 2)) at 2 mg/kg. All of the LNPs were formulated with ionizable lipid 10b-15. The mice were dosed every other day for a total of 5 doses. Nalm6 tumor burden over time was quantified using IVIS imaging.

As seen in FIG. 99, LNPs formulated with CD19-CD28ζCARs are more effective in tumor regression than the other CAR or controls (positive or negative).

### Example 85

Expression of gLuc oRNA by Novel IRESs in Primary Human Hepatocytes, Primary Human Myotubes, and Primary Human T Cells

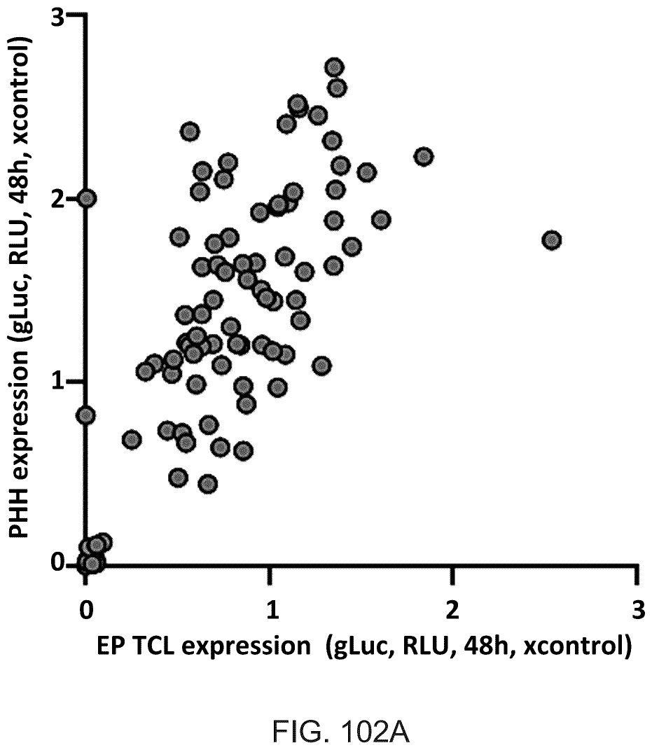

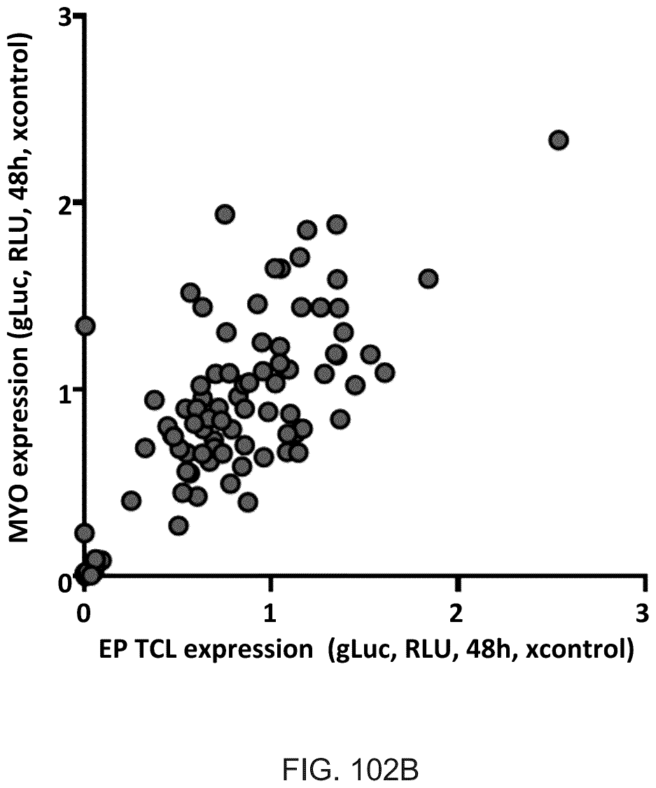

93 constructs, each containing a unique IRES sequence preceding (i.e., 5′ to) a Gaussia luciferase (gLuc) expression sequence, were synthesized. Circular RNA (oRNA) was encapsulated in Lipid A and transfected into primary human hepatocytes (50 ng/well) and primary human myotubes (100 ng/well). Primary human T cells were transfected with 5 ng/well of non-formulated oRNA via electroporation. At 48 hours, the cell supernatant was collected and read for luminescence. IRES-driven gLuc expression was then normalized to an internal control IRES having SEQ ID NO: 3282 that was transfected in triplicate on each plate to determine relative expression levels. The data comparing expression in T cells electroporated with a gLuc-encoding construct as compared to that of hepatocytes and myotubes show an increase in correlation as compared to T cell expression when gLuc-encoding constructs were delivered by a lipid nanoparticle (LNP) (see Tables 5 & 6 and FIGS. 102A & 102B).

TABLE 5
Comparison of expression of gLuc in T cells
transfected by electroporation or primary
human hepatocytes transfected with LNPs

| Construct | T cells | Hepatocytes |
|---|---|---|
| C1 | 0.781812 | 1.789967 |
| (SEQ ID NO: 693) | | |
| C2 | 0.843272 | 1.20282 |
| (SEQ ID NO: 752) | | |
| C3 | 1.084859 | 1.685733 |
| (SEQ ID NO: 785) | | |
| C4 | 0.875589 | 0.880832 |
| (SEQ ID NO: 787) | | |
| C5 | 0.702179 | 1.755608 |
| (SEQ ID NO: 791) | | |
| C6 | 1.530501 | 2.144337 |
| (SEQ ID NO: 793) | | |
| C7 | 0.669549 | 0.769599 |
| (SEQ ID NO: 820) | | |
| C8 | 1.350993 | 1.881923 |
| (SEQ ID NO: 823) | | |
| C9 | 0.823706 | 1.209531 |
| (SEQ ID NO: 839) | | |
| C10 | 1.101436 | 1.978986 |
| (SEQ ID NO: 840) | | |
| C11 | 0.69405 | 1.209325 |
| (SEQ ID NO: 843) | | |
| C12 | 0.632273 | 1.629583 |
| (SEQ ID NO: 852) | | |
| C13 | 1.38681 | 2.181173 |
| (SEQ ID NO: 857) | | |
| C14 | 1.160055 | 2.493854 |
| (SEQ ID NO: 861) | | |
| C15 | 1.093723 | 2.409856 |
| (SEQ ID NO: 862) | | |
| C16 | 0.636014 | 2.150293 |
| (SEQ ID NO: 863) | | |
| C17 | 1.131199 | 2.03697 |
| (SEQ ID NO: 864) | | |
| C18 | 0.85826 | 0.979795 |
| (SEQ ID NO: 874) | | |
| C19 | 1.607973 | 1.885157 |
| (SEQ ID NO: 876) | | |
| C20 | 0.695179 | 1.448789 |
| (SEQ ID NO: 959) | | |
| C21 | 1.145246 | 1.449374 |
| (SEQ ID NO: 983) | | |
| C22 | 0.789058 | 1.303281 |
| (SEQ ID NO: 984) | | |
| C23 | 0.740537 | 1.094131 |
| (SEQ ID NO: 1015) | | |
| C24 | 1.450209 | 1.73917 |
| (SEQ ID NO: 1017) | | |
| C25 | 1.284727 | 1.090183 |
| (SEQ ID NO: 1023) | | |
| C26 | 0.541319 | 1.36819 |
| (SEQ ID NO: 1026) | | |
| C27 | 0.00527 | 2.002515 |
| (SEQ ID NO: 1031) | | |
| C28 | 0.548442 | 1.214363 |
| (SEQ ID NO: 1041) | | |
| C29 | 0.925584 | 1.65123 |
| (SEQ ID NO: 1047) | | |
| C30 | 0.250089 | 0.687958 |
| (SEQ ID NO: 1059) | | |
| C31 | 0.775688 | 2.198663 |
| (SEQ ID NO: 1068) | | |
| C32 | 0.63106 | 1.194958 |
| (SEQ ID NO: 1134) | | |
| C33 | 1.046387 | 0.972607 |
| (SEQ ID NO: 1168) | | |
| C34 | 1.34302 | 2.317664 |
| (SEQ ID NO: 1169) | | |
| C35 | 1.369419 | 2.604943 |
| (SEQ ID NO: 1171) | | |
| C36 | 0.75288 | 2.10632 |
| (SEQ ID NO: 1177) | | |
| C37 | 0.510432 | 1.792991 |
| (SEQ ID NO: 1178) | | |
| C38 | 1.167913 | 1.339824 |
| (SEQ ID NO: 1179) | | |
| C39 | 0.9501 | 1.926039 |
| (SEQ ID NO: 1180) | | |
| C40 | 0.855226 | 1.644577 |
| (SEQ ID NO: 1189) | | |
| C41 | 1.360869 | 2.049345 |
| (SEQ ID NO: 1192) | | |
| C42 | 0.960246 | 1.204445 |
| (SEQ ID NO: 1193) | | |
| C43 | 0.881406 | 1.560716 |
| (SEQ ID NO: 1198) | | |
| C44 | 1.840964 | 2.230125 |
| (SEQ ID NO: 1216) | | |
| C45 | 0.469295 | 1.047787 |
| (SEQ ID NO: 1263) | | |
| C46 | 0.568126 | 2.36749 |
| (SEQ ID NO: 1276) | | |
| C47 | 0.632638 | 1.373936 |
| (SEQ ID NO: 1280) | | |
| C48 | 0.375273 | 1.101024 |
| (SEQ ID NO: 1282) | | |
| C49 | 1.264343 | 2.456285 |
| (SEQ ID NO: 1284) | | |
| C50 | 1.045813 | 1.955882 |
| (SEQ ID NO: 1287) | | |
| C51 | 0.601175 | 0.989625 |
| (SEQ ID NO: 3292) | | |
| C52 | 1.022958 | 1.443859 |
| (SEQ ID NO: 3293) | | |
| C53 | 0.955833 | 1.505989 |
| (SEQ ID NO: 3294) | | |
| C54 | 0.718225 | 1.640835 |
| (SEQ ID NO: 3295) | | |
| C55 | 0.092929 | 0.127566 |
| (SEQ ID NO: 3296) | | |
| C56 | 0.665661 | 0.447235 |
| (SEQ ID NO: 3297) | | |
| C57 | 1.152769 | 2.517318 |
| (SEQ ID NO: 2285) | | |
| C58 | 0.0014 | 0.002206 |
| (SEQ ID NO: 2601) | | |
| C59 | 0.051441 | 0.042088 |
| (SEQ ID NO: 2615) | | |
| C60 | 0.008382 | 0.030507 |
| (SEQ ID NO: 2616) | | |
| C61 | 0.007267 | 0.002422 |
| (SEQ ID NO: 2617) | | |
| C62 | 0.008072 | 0.021991 |
| (SEQ ID NO: 2618) | | |
| C63 | 0.000761 | 0 |
| (SEQ ID NO: 2627) | | |
| C64 | 0.564561 | 1.198385 |
| (SEQ ID NO: 871) | | |
| C65 | 0.733764 | 0.646334 |
| (SEQ ID NO: 922) | | |
| C66 | 0.016487 | 0.10179 |
| (SEQ ID NO: 2667) | | |
| C67 | 0.058548 | 0.015293 |
| (SEQ ID NO: 2681) | | |
| C68 | 1.088459 | 1.151406 |
| (SEQ ID NO: 2742) | | |
| C69 | 0.587669 | 1.156564 |
| (SEQ ID NO: 2746) | | |
| C70 | 0.325456 | 1.059696 |
| (SEQ ID NO: 2758) | | |
| C71 | 1.050785 | 1.969435 |
| (SEQ ID NO: 2777) | | |
| C72 | 1.350472 | 1.635228 |
| (SEQ ID NO: 2778) | | |
| C73 | 0.857241 | 0.628088 |
| (SEQ ID NO: 3298) | | |
| C74 | 0.445517 | 0.737632 |
| (SEQ ID NO: 3299) | | |
| C75 | 0.000962 | 0.820587 |
| (SEQ ID NO: 3300) | | |
| C76 | 0.479121 | 1.125387 |
| (SEQ ID NO: 3301) | | |
| C77 | 1.01908 | 1.169268 |
| (SEQ ID NO: 3302) | | |
| C78 | 1.35322 | 2.716134 |
| (SEQ ID NO: 3283) | | |
| C79 | 0.621993 | 2.039883 |
| (SEQ ID NO: 3284) | | |
| C80 | 0.604474 | 1.253068 |
| (SEQ ID NO: 3285) | | |
| C81 | 0.503822 | 0.481752 |
| (SEQ ID NO: 75) | | |
| C82 | 0.526345 | 0.723817 |
| (SEQ ID NO: 77) | | |
| C83 | 0.004556 | 0 |
| (SEQ ID NO: 137) | | |
| C84 | 0.546897 | 0.671198 |
| (SEQ ID NO: 3289) | | |
| C85 | 1.191477 | 1.603127 |
| (SEQ ID NO: 3290) | | |
| C86 | 2.538039 | 1.774949 |
| (SEQ ID NO: 3291) | | |
| C87 | 0.059416 | 0.114372 |
| (SEQ ID NO: 532) | | |
| C88 | 0.036736 | 0.011773 |
| (SEQ ID NO: 566) | | |
| C89 | 0.760741 | 1.602763 |
| (SEQ ID NO: 580) | | |
| C90 | 0.983704 | 1.461777 |
| (SEQ ID NO: 648) | | |

TABLE 6
Comparison of expression of gLuc in T cells
transfected by electroporation or primary
human myotubes transfected with LNPs

| Construct | T cells | Myotbubes |
|---|---|---|
| C1 | 0.781812 | 0.4959 |
| (SEQ ID NO: 693) | | |
| C2 | 0.843272 | 0.588222 |
| (SEQ ID NO: 752) | | |
| C3 | 1.084859 | 0.6651 |
| (SEQ ID NO: 785) | | |
| C4 | 0.875589 | 0.396423 |
| (SEQ ID NO: 787) | | |
| C5 | 0.702179 | 1.082975 |
| (SEQ ID NO: 791) | | |
| C6 | 1.530501 | 1.186985 |
| (SEQ ID NO:s 793) | | |
| C7 | 0.669549 | 0.613586 |
| (SEQ ID NO: 820) | | |
| C8 | 1.350993 | 1.181903 |
| (SEQ ID NO: 823) | | |
| C9 | 0.823706 | 0.961742 |
| (SEQ ID NO: 839) | | |
| C10 | 1.101436 | 0.868987 |
| (SEQ ID NO: 840) | | |
| C11 | 0.69405 | 0.727779 |
| (SEQ ID NO: 843) | | |
| C12 | 0.632273 | 0.95248 |
| (SEQ ID NO: 852) | | |
| C13 | 1.38681 | 1.306163 |
| (SEQ ID NO: 857) | | |
| C14 | 1.160055 | 1.442309 |
| (SEQ ID NO: 861) | | |
| C15 | 1.093723 | 1.106511 |
| (SEQ ID NO: 862) | | |
| C16 | 0.636014 | 0.788138 |
| (SEQ ID NO: 863) | | |
| C17 | 1.131199 | 0.765271 |
| (SEQ ID NO: 864) | | |
| C18 | 0.85826 | 0.700623 |
| (SEQ ID NO: 874) | | |
| C19 | 1.607973 | 1.090032 |
| (SEQ ID NO: 876) | | |
| C20 | 0.695179 | 0.686847 |
| (SEQ ID NO: 959) | | |
| C21 | 1.145246 | 0.662146 |
| (SEQ ID NO: 983) | | |
| C22 | 0.789058 | 0.786323 |
| (SEQ ID NO: 984) | | |
| C23 | 0.740537 | 0.657684 |
| (SEQ ID NO: 1015) | | |
| C24 | 1.450209 | 1.02179 |
| (SEQ ID NO: 1017) | | |
| C25 | 1.284727 | 1.082823 |
| (SEQ ID NO: 1023) | | |
| C26 | 0.541319 | 0.896722 |
| (SEQ ID NO: 1026) | | |
| C27 | 0.00527 | 1.341548 |
| (SEQ ID NO: 1031) | | |
| C28 | 0.548442 | 0.658859 |
| (SEQ ID NO: 1041) | | |
| C29 | 0.925584 | 1.458831 |
| (SEQ ID NO: 1047) | | |
| C30 | 0.250089 | 0.403819 |
| (SEQ ID NO: 1059) | | |
| C31 | 0.775688 | 1.086343 |
| (SEQ ID NO: 1068) | | |
| C32 | 0.63106 | 0.6557 |
| (SEQ ID NO: 1134) | | |
| C33 | 1.046387 | 1.228291 |
| (SEQ ID NO: 1168) | | |
| C34 | 1.34302 | 1.190566 |
| (SEQ ID NO: 1169) | | |
| C35 | 1.369419 | 0.84109 |
| (SEQ ID NO: 1171) | | |
| C36 | 0.75288 | 1.937973 |
| (SEQ ID NO: 1177) | | |
| C37 | 0.510432 | 0.681991 |
| (SEQ ID NO: 1178) | | |
| C38 | 1.167913 | 0.788823 |
| (SEQ ID NO: 1179) | | |
| C39 | 0.9501 | 1.25303 |
| (SEQ ID NO: 1180) | | |
| C40 | 0.855226 | 1.026352 |
| (SEQ ID NO: 1189) | | |
| C41 | 1.360869 | 1.436999 |
| (SEQ ID NO: 1192) | | |
| C42 | 0.960246 | 0.636939 |
| (SEQ ID NO: 1193) | | |
| C43 | 0.881406 | 1.035781 |
| (SEQ ID NO: 1198) | | |
| C44 | 1.840964 | 1.593485 |
| (SEQ ID NO: 1216) | | |
| C45 | 0.469295 | 0.75519 |
| (SEQ ID NO: 1263) | | |
| C46 | 0.568126 | 1.519461 |
| (SEQ ID NO: 1276) | | |
| C47 | 0.632638 | 1.441776 |
| (SEQ ID NO: 1280) | | |
| C48 | 0.375273 | 0.943528 |
| (SEQ ID NO: 1282) | | |
| C49 | 1.264343 | 1.439882 |
| (SEQ ID NO: 1284) | | |
| C50 | 1.045813 | 1.138229 |
| (SEQ ID NO: 1287) | | |
| C51 | 0.601175 | 0.897061 |
| (SEQ ID NO: 3292) | | |
| C52 | 1.022958 | 1.03281 |
| (SEQ ID NO: 3293) | | |
| C53 | 0.955833 | 1.097108 |
| (SEQ ID NO: 3294) | | |
| C54 | 0.718225 | 0.901887 |
| (SEQ ID NO: 3295) | | |
| C55 | 0.092929 | 0.084114 |
| (SEQ ID NO: 3296) | | |
| C56 | 0.665661 | 0.843353 |
| (SEQ ID NO: 3297) | | |
| C57 | 1.152769 | 1.708846 |
| (SEQ ID NO: 2285) | | |
| C58 | 0.0014 | 0.014801 |
| (SEQ ID NO: 2601) | | |
| C59 | 0.051441 | 0.024369 |
| (SEQ ID NO: 2615) | | |
| C60 | 0.008382 | 0.013367 |
| (SEQ ID NO: 2616) | | |
| C61 | 0.007267 | 0 |
| (SEQ ID NO: 2617) | | |
| C62 | 0.008072 | 0.022575 |
| (SEQ ID NO: 2618) | | |
| C63 | 0.000761 | 0 |
| (SEQ ID NO: 2627) | | |
| C64 | 0.564561 | 0.55265 |
| (SEQ ID NO: 871) | | |
| C65 | 0.733764 | 0.833352 |
| (SEQ ID NO: 922) | | |
| C66 | 0.016487 | 0.025211 |
| (SEQ ID NO: 2667) | | |
| C67 | 0.058548 | 0.060367 |
| (SEQ ID NO: 2681) | | |
| C68 | 1.088459 | 0.761185 |
| (SEQ ID NO: 2742) | | |
| C69 | 0.587669 | 0.816852 |
| (SEQ ID NO: 2746) | | |
| C70 | 0.325456 | 0.68701 |
| (SEQ ID NO: 2758) | | |
| C71 | 1.050785 | 1.647834 |
| (SEQ ID NO: 2777) | | |
| C72 | 1.350472 | 1.884789 |
| (SEQ ID NO: 2778) | | |
| C73 | 0.857241 | 0.897388 |
| (SEQ ID NO: 3298) | | |
| C74 | 0.445517 | 0.802662 |
| (SEQ ID NO: 3299) | | |
| C75 | 0.000962 | 0.230752 |
| (SEQ ID NO: 3300) | | |
| C76 | 0.479121 | 0.748166 |
| (SEQ ID NO: 3301) | | |
| C77 | 1.01908 | 1.649437 |
| (SEQ ID NO: 3302) | | |
| C78 | 1.35322 | 1.590997 |
| (SEQ ID NO: 3283) | | |
| C79 | 0.621993 | 1.020461 |
| (SEQ ID NO: 3284) | | |
| C80 | 0.604474 | 0.427239 |
| (SEQ ID NO: 3285) | | |
| C81 | 0.503822 | 0.271581 |
| (SEQ ID NO: 75) | | |
| C82 | 0.526345 | 0.447674 |
| (SEQ ID NO: 77) | | |
| C83 | 0.004556 | 0 |
| (SEQ ID NO: 137) | | |
| C84 | 0.546897 | 0.560942 |
| (SEQ ID NO: 3289) | | |
| C85 | 1.191477 | 1.854881 |
| (SEQ ID NO: 3290) | | |
| C86 | 2.538039 | 2.338184 |
| (SEQ ID NO: 3291) | | |
| C87 | 0.059416 | 0.09047 |
| (SEQ ID NO: 532) | | |
| C88 | 0.036736 | 0.005766 |
| (SEQ ID NO: 566) | | |
| C89 | 0.760741 | 1.306589 |
| (SEQ ID NO: 580) | | |
| C90 | 0.983704 | 0.880306 |
| (SEQ ID NO: 648) | | |

### Example 86

Expression of gLuc and FLuc Coding Regions by Novel IRESs in Primary Human Hepatocytes, Primary Human Myotubes, and Primary Human T Cells

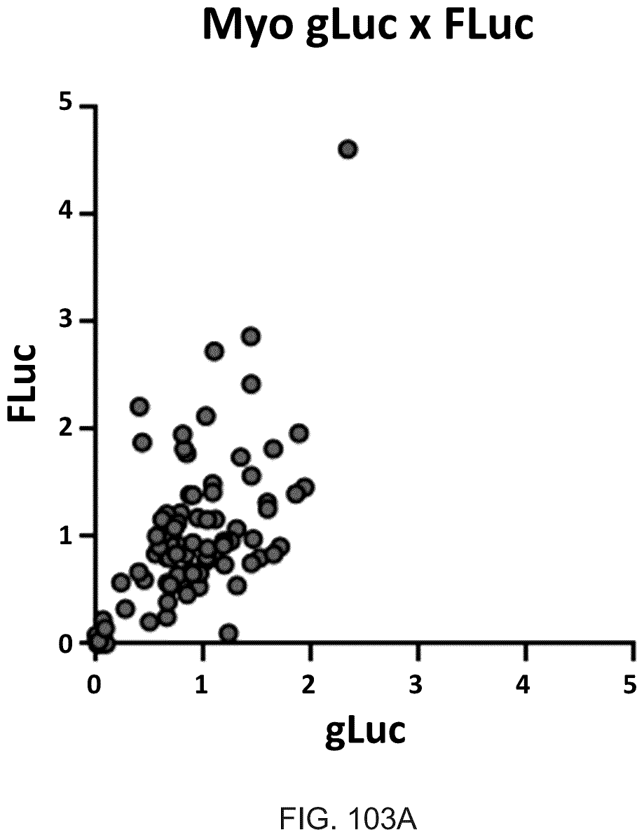

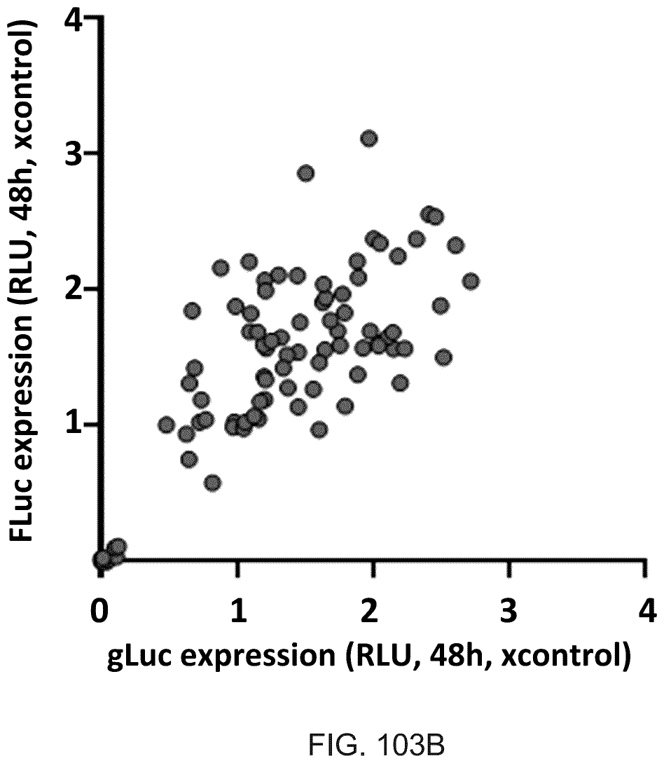

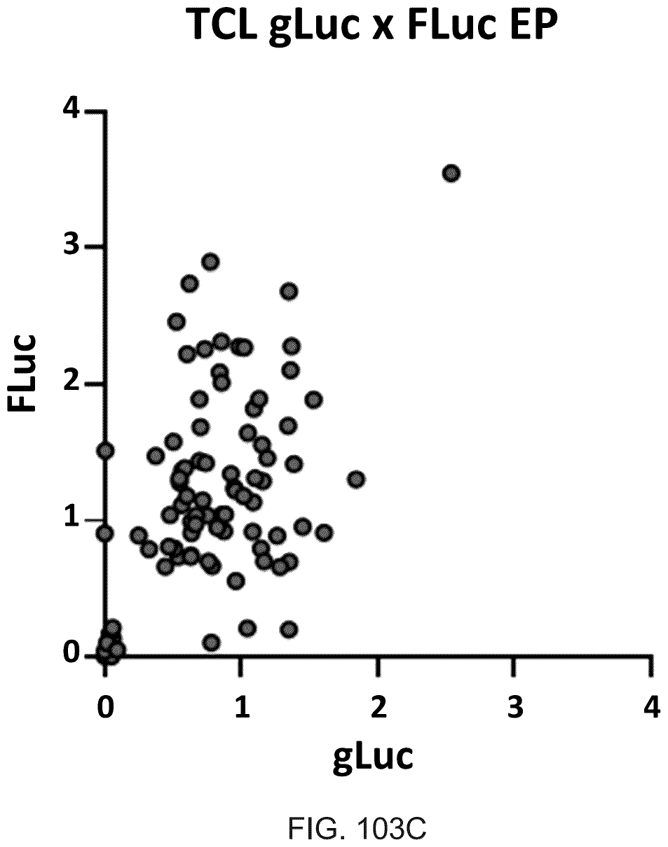

93 constructs, each containing a unique IRES sequence preceding a Firefly luciferase (FLuc) expression sequence, were synthesized. oRNA was encapsulated in Lipid A and transfected into primary human hepatocytes (50 ng/well) and primary human myotubes (100 ng/well). Primary human T cells were transfected via electroporation with 5 ng/well of non-formulated oRNA. At 48 hours, the cell supernatant was collected and read for luminescence. IRES-mediated FLuc expression was then normalized to an internal control (SEQ ID NO: 3282) that was transfected in triplicate on each plate to determine relative expression levels. The FLuc expression data was compared to the gLuc expression data shown in Example 1 to assess IRES-mediated efficacy in: (1) mediating expression of coding regions of different lengths; and (2) producing both secreted (gLuc) and intracellular (FLuc) proteins within the three tested cell types. The findings of these experiments demonstrate that there is a positive correlation between gLuc and FLuc expression in all three cell types, indicating that selected IRES sequences can drive robust expression of different target genes (see Tables 7-9 and FIGS. 103A-103C).

TABLE 7
Comparison of gLuc and FLuc expression
data - Primary human myotubes

| Construct | gLuc | FLuc |
|---|---|---|
| C78 | 1.590997 | 1.312724 |
| (SEQ ID NO: 3283) | | |
| C91 | 0.95248 | 0.531801 |
| (SEQ ID NO: 852) | | |
| C13 | 1.306163 | 1.071016 |
| (SEQ ID NO: 857) | | |
| C14 | 1.442309 | 1.562547 |
| (SEQ ID NO: 861) | | |
| C15 | 1.106511 | 1.157547 |
| (SEQ ID NO: 862) | | |
| C16 | 0.788138 | 0.595526 |
| (SEQ ID NO: 863) | | |
| C17 | 0.765271 | 0.917029 |
| (SEQ ID NO: 864) | | |
| C64 | 0.55265 | 0.837934 |
| (SEQ ID NO: 871) | | |
| C18 | 0.700623 | 0.549536 |
| (SEQ ID NO: 874) | | |
| C19 | 1.090032 | 0.836932 |
| (SEQ ID NO: 876) | | |
| C65 | 0.833352 | 0.832225 |
| (SEQ ID NO: 922) | | |
| C20 | 0.686847 | 0.927862 |
| (SEQ ID NO: 959) | | |
| C21 | 0.662146 | 0.569056 |
| (SEQ ID NO: 983) | | |
| C22 | 0.786323 | 1.216579 |
| (SEQ ID NO: 984) | | |
| C23 | 0.657684 | 1.209252 |
| (SEQ ID NO: 1015) | | |
| C24 | 1.02179 | 0.77457 |
| (SEQ ID NO: 1017) | | |
| C25 | 1.082823 | 1.487919 |
| (SEQ ID NO: 1023) | | |
| C26 | 0.896722 | 0.939609 |
| (SEQ ID NO: 1026) | | |
| C92 | 1.341548 | 1.73804 |
| (SEQ ID NO: 1037) | | |
| C28 | 0.658859 | 0.803578 |
| (SEQ ID NO: 1041) | | |
| C29 | 1.458831 | 0.976716 |
| (SEQ ID NO: 1047) | | |
| C30 | 0.403819 | 2.208373 |
| (SEQ ID NO: 1059) | | |
| C31 | 1.086343 | 1.486304 |
| (SEQ ID NO: 1068) | | |
| C32 | 0.6557 | 0.248399 |
| (SEQ ID NO: 1134) | | |
| C33 | 1.228291 | 0.097162 |
| (SEQ ID NO: 1168) | | |
| C34 | 1.190566 | 0.738396 |
| (SEQ ID NO: 1169) | | |
| C35 | 0.84109 | 1.771565 |
| (SEQ ID NO: 1171) | | |
| C36 | 1.937973 | 1.454313 |
| (SEQ ID NO: 1177) | | |
| C37 | 0.681991 | 0.797131 |
| (SEQ ID NO: 1178) | | |
| C38 | 0.788823 | 0.596369 |
| (SEQ ID NO: 1179) | | |
| C39 | 1.25303 | 0.95716 |
| (SEQ ID NO: 1180) | | |
| C40 | 1.026352 | 0.8111 |
| (SEQ ID NO: 1189) | | |
| C41 | 1.436999 | 2.857461 |
| (SEQ ID NO: 1192) | | |
| C42 | 0.636939 | 1.031387 |
| (SEQ ID NO: 1193) | | |
| C43 | 1.035781 | 0.887593 |
| (SEQ ID NO: 1198) | | |
| C44 | 1.593485 | 1.253139 |
| (SEQ ID NO: 1216) | | |
| C45 | 0.75519 | 1.124883 |
| (SEQ ID NO: 1263) | | |
| C46 | 1.519461 | 0.799936 |
| (SEQ ID NO: 1276) | | |
| C47 | 1.441776 | 0.751532 |
| (SEQ ID NO: 1280) | | |
| C48 | 0.943528 | 1.173641 |
| (SEQ ID NO: 1282) | | |
| C49 | 1.439882 | 2.416924 |
| (SEQ ID NO: 1284) | | |
| C57 | 1.708846 | 0.903192 |
| (SEQ ID NO: 2285) | | |
| C93 | 0.271581 | 0.327022 |
| (SEQ ID NO: 75) | | |
| C94 | 0.447674 | 0.596422 |
| (SEQ ID NO: 77) | | |
| C58 | 0.014801 | 0.003874 |
| (SEQ ID NO: 2601) | | |
| C59 | 0.024369 | 0 |
| (SEQ ID NO: 2615) | | |
| C60 | 0.013367 | 0.033495 |
| (SEQ ID NO: 2616) | | |
| C61 | 0 | 0 |
| (SEQ ID NO: 2617) | | |
| C62 | 0.022575 | 0 |
| (SEQ ID NO: 2618) | | |
| C63 | 0 | 0.079153 |
| (SEQ ID NO: 2627) | | |
| C83 | 0 | 0 |
| (SEQ ID NO: 137) | | |
| C87 | 0.09047 | 0 |
| (SEQ ID NO: 532) | | |
| C88 | 0.005766 | 0 |
| (SEQ ID NO: 566) | | |
| C89 | 1.306589 | 0.544577 |
| (SEQ ID NO: 580) | | |
| C90 | 0.880306 | 0.673771 |
| (SEQ ID NO: 648) | | |
| C1 | 0.4959 | 0.203921 |
| (SEQ ID NO: 693) | | |
| C2 | 0.588222 | 0.903914 |
| (SEQ ID NO: 752) | | |
| C3 | 0.6651 | 0.389754 |
| (SEQ ID NO: 785) | | |
| C4 | 0.396423 | 0.668529 |
| (SEQ ID NO: 787) | | |
| IRC1-947 | 1.082975 | 1.408435 |
| (SEQ ID NO: 791) | | |
| C5 | 1.186985 | 0.953863 |
| (SEQ ID NO: 793) | | |
| C7 | 0.613586 | 1.156583 |
| (SEQ ID NO: 820) | | |
| C8 | 1.181903 | 0.916452 |
| (SEQ ID NO: 823) | | |
| C9 | 0.961742 | 0.657186 |
| (SEQ ID NO: 839) | | |
| C10 | 0.868987 | 1.38723 |
| (SEQ ID NO: 840) | | |
| C11 | 0.727779 | 1.078516 |
| (SEQ ID NO: 841) | | |
| C66 | 0.025211 | 0.027435 |
| (SEQ ID NO: 2667) | | |
| C67 | 0.060367 | 0.224418 |
| (SEQ ID NO: 2681) | | |
| C68 | 0.761185 | 0.639208 |
| (SEQ ID NO: 2742) | | |
| C69 | 0.816852 | 1.815435 |
| (SEQ ID NO: 2746) | | |
| C70 | 0.68701 | 0.549928 |
| (SEQ ID NO: 2758) | | |
| C71 | 1.647834 | 1.814036 |
| (SEQ ID NO: 2777) | | |
| C72 | 1.884789 | 1.960701 |
| (SEQ ID NO: 2778) | | |
| C84 | 0.560942 | 1.00269 |
| (SEQ ID NO: 3289) | | |
| C85 | 1.854881 | 1.396289 |
| (SEQ ID NO: 3290) | | |
| C95 | 2.338184 | 4.600078 |
| (SEQ ID NO: 3288) | | |
| C51 | 0.897061 | 1.384093 |
| (SEQ ID NO: 3292) | | |
| C52 | 1.03281 | 1.155681 |
| (SEQ ID NO: 3293) | | |
| C53 | 1.097108 | 2.720138 |
| (SEQ ID NO: 3294) | | |
| C54 | 0.901887 | 0.93398 |
| (SEQ ID NO: 3295) | | |
| C55 | 0.084114 | 0.142769 |
| (SEQ ID NO: 3296) | | |
| C56 | 0.843353 | 0.460966 |
| (SEQ ID NO: 3297) | | |
| C73 | 0.897388 | 0.653462 |
| (SEQ ID NO: 3298) | | |
| C74 | 0.802662 | 1.949759 |
| (SEQ ID NO: 3299) | | |
| C75 | 0.230752 | 0.573199 |
| (SEQ ID NO: 3300) | | |
| C76 | 0.748166 | 0.835379 |
| (SEQ ID NO: 3301) | | |
| C77 | 1.649437 | 0.836293 |
| (SEQ ID NO: 3302) | | |
| C79 | 1.020461 | 2.118603 |
| (SEQ ID NO: 3284 | | |
| C80 | 0.427239 | 1.876227 |
| (SEQ ID NO: 3285) | | |

TABLE 8
Comparison of gLuc and FLuc expression
data - Primary human hepatocytes

| Construct | gLuc | FLuc |
|---|---|---|
| C78 | 2.716134 | 2.060788 |
| (SEQ ID NO: 3283) | | |
| C91 | 1.629583 | 1.905209 |
| (SEQ ID NO: 852) | | |
| C13 | 2.181173 | 2.246248 |
| (SEQ ID NO: 857) | | |
| C14 | 2.493854 | 1.881799 |
| (SEQ ID NO: 861) | | |
| C15 | 2.409856 | 2.552247 |
| (SEQ ID NO: 862) | | |
| C16 | 2.150293 | 1.562611 |
| (SEQ ID NO: 863) | | |
| C17 | 2.03697 | 1.603392 |
| (SEQ ID NO: 864) | | |
| C64 | 1.198385 | 1.359501 |
| (SEQ ID NO: 871) | | |
| C18 | 0.979795 | 1.02569 |
| (SEQ ID NO: 874) | | |
| C19 | 1.885157 | 1.374759 |
| (SEQ ID NO: 876) | | |
| C65 | 0.646334 | 0.752749 |
| (SEQ ID NO: 922) | | |
| C20 | 1.448789 | 1.538369 |
| (SEQ ID NO: 959) | | |
| C21 | 1.449374 | 1.136936 |
| (SEQ ID NO: 983) | | |
| C22 | 1.303281 | 2.10726 |
| (SEQ ID NO: 984) | | |
| C23 | 1.094131 | 1.689963 |
| (SEQ ID NO: 1015) | | |
| C24 | 1.73917 | 1.696402 |
| (SEQ ID NO: 1017) | | |
| C25 | 1.090183 | 2.204761 |
| (SEQ ID NO: 1023) | | |
| C26 | 1.36819 | 1.51613 |
| (SEQ ID NO: 1026) | | |
| C92 | 2.002515 | 2.372148 |
| (SEQ ID NO: 1037) | | |
| C28 | 1.214363 | 1.565902 |
| (SEQ ID NO: 1041) | | |
| C29 | 1.65123 | 1.938792 |
| (SEQ ID NO: 1047) | | |
| C30 | 0.687958 | 1.421279 |
| (SEQ ID NO: 1059) | | |
| C31 | 2.198663 | 1.311447 |
| (SEQ ID NO: 1068) | | |
| C32 | 1.194958 | 1.591302 |
| (SEQ ID NO: 1134) | | |
| C33 | 0.972607 | 0.987267 |
| (SEQ ID NO: 1168) | | |
| C34 | 2.317664 | 2.370785 |
| (SEQ ID NO: 1169) | | |
| C35 | 2.604943 | 2.325792 |
| (SEQ ID NO: 1171) | | |
| C36 | 2.10632 | 1.649129 |
| (SEQ ID NO: 1177) | | |
| C37 | 1.792991 | 1.141286 |
| (SEQ ID NO: 1178) | | |
| C38 | 1.339824 | 1.421089 |
| (SEQ ID NO: 1179) | | |
| C39 | 1.926039 | 1.567946 |
| (SEQ ID NO: 1180) | | |
| C40 | 1.644577 | 1.555664 |
| (SEQ ID NO: 1189) | | |
| C41 | 2.049345 | 2.33941 |
| (SEQ ID NO: 1192) | | |
| C42 | 1.204445 | 2.06872 |
| (SEQ ID NO: 1193) | | |
| C43 | 1.560716 | 1.265724 |
| (SEQ ID NO: 1198) | | |
| C44 | 2.230125 | 1.564512 |
| (SEQ ID NO: 1216) | | |
| C45 | 1.047787 | 0.979048 |
| (SEQ ID NO: 1263) | | |
| C46 | 1.891742 | 2.088992 |
| (SEQ ID NO: 1276) | | |
| C47 | 1.373936 | 1.276431 |
| (SEQ ID NO: 1280) | | |
| C48 | 1.101024 | 1.823506 |
| (SEQ ID NO: 1282) | | |
| C49 | 2.456285 | 2.533504 |
| (SEQ ID NO: 1284) | | |
| C57 | 2.517318 | 1.499781 |
| (SEQ ID NO: 2285) | | |
| C93 | 0.481752 | 1.004326 |
| (SEQ ID NO: 75) | | |
| C94 | 0.723817 | 1.023214 |
| (SEQ ID NO: 77) | | |
| C58 | 0.002206 | 0 |
| (SEQ ID NO: 2601) | | |
| C59 | 0.042088 | 0 |
| (SEQ ID NO: 2615) | | |
| C60 | 0.045761 | 0 |
| (SEQ ID NO: 2616) | | |
| C61 | 0.002422 | 0 |
| (SEQ ID NO: 2617) | | |
| C62 | 0.055067 | 0 |
| (SEQ ID NO: 2618) | | |
| C63 | 0 | 0.016989 |
| (SEQ ID NO: 2627) | | |
| C83 | 0 | 0.007678 |
| (SEQ ID NO: 137) | | |
| C87 | 0.114372 | 0.033199 |
| (SEQ ID NO: 532) | | |
| C88 | 0.011773 | 0.000778 |
| (SEQ ID NO: 566) | | |
| C89 | 1.602763 | 0.969482 |
| (SEQ ID NO: 580) | | |
| C90 | 1.461777 | 1.759526 |
| (SEQ ID NO: 648) | | |
| C1 | 1.789967 | 1.829363 |
| (SEQ ID NO: 693) | | |
| C2 | 1.20282 | 1.18851 |
| (SEQ ID NO: 752) | | |
| C3 | 1.685733 | 1.772856 |
| (SEQ ID NO: 785) | | |
| C4 | 0.880832 | 2.158202 |
| (SEQ ID NO: 787) | | |
| IRC1-947 | 1.755608 | 1.587946 |
| (SEQ ID NO: 791) | | |
| C5 | 2.144337 | 1.684434 |
| (SEQ ID NO: 793) | | |
| C7 | 0.769599 | 1.041986 |
| (SEQ ID NO: 820) | | |
| C8 | 1.881923 | 2.208252 |
| (SEQ ID NO: 823) | | |
| C9 | 1.209531 | 1.990984 |
| (SEQ ID NO: 839) | | |
| C10 | 1.978986 | 1.694731 |
| (SEQ ID NO: 840) | | |
| C11 | 1.209325 | 1.335431 |
| (SEQ ID NO: 841) | | |
| C66 | 0.10179 | 0.098907 |
| (SEQ ID NO: 2667) | | |
| C67 | 0.015293 | 0.025525 |
| (SEQ ID NO: 2681) | | |
| C68 | 1.151406 | 1.686267 |
| (SEQ ID NO: 2742) | | |
| C69 | 1.156564 | 1.048837 |
| (SEQ ID NO: 2746) | | |
| C70 | 1.059696 | 1.022913 |
| (SEQ ID NO: 2758) | | |
| C71 | 1.969435 | 3.111428 |
| (SEQ ID NO: 2777) | | |
| C72 | 1.635228 | 2.038087 |
| (SEQ ID NO: 2778) | | |
| C84 | 0.671198 | 1.845161 |
| (SEQ ID NO: 3289) | | |
| C85 | 1.603127 | 1.462123 |
| (SEQ ID NO: 3290) | | |
| C95 | 1.774949 | 1.966788 |
| (SEQ ID NO: 3288) | | |
| C51 | 0.989625 | 1.875905 |
| (SEQ ID NO: 3292) | | |
| C52 | 1.443859 | 2.102649 |
| (SEQ ID NO: 3293) | | |
| C53 | 1.505989 | 2.853261 |
| (SEQ ID NO: 3294) | | |
| C54 | 1.322062 | 1.64633 |
| (SEQ ID NO: 3295) | | |
| C55 | 0.127566 | 0.11069 |
| (SEQ ID NO: 3296) | | |
| C56 | 0.649718 | 1.310716 |
| (SEQ ID NO: 3297) | | |
| C73 | 0.628088 | 0.936313 |
| (SEQ ID NO: 3298) | | |
| C74 | 0.737632 | 1.187904 |
| (SEQ ID NO: 3299) | | |
| C75 | 0.820587 | 0.579661 |
| (SEQ ID NO: 3300) | | |
| C76 | 1.125387 | 1.069279 |
| (SEQ ID NO: 3301) | | |
| C77 | 1.169268 | 1.173599 |
| (SEQ ID NO: 3302) | | |
| C79 | 2.039883 | 1.583805 |
| (SEQ ID NO: 3284) | | |
| C80 | 1.253068 | 1.620713 |
| (SEQ ID NO: 3285) | | |

TABLE 9
Comparison of gLuc and FLuc expression
data - Primary human T cells

| Construct | gLuc | FLuc |
|---|---|---|
| C78 | 1.35322 | 0.694504 |
| (SEQ ID NO: 3283) | | |
| C91 | 0.632273 | 0.741893 |
| (SEQ ID NO: 852) | | |
| C13 | 1.38681 | 1.413992 |
| (SEQ ID NO: 857) | | |
| C14 | 1.160055 | 1.287027 |
| (SEQ ID NO: 861) | | |
| C15 | 1.093723 | 1.820246 |
| (SEQ ID NO: 862) | | |
| C16 | 0.636014 | 0.907711 |
| (SEQ ID NO: 863) | | |
| C17 | 1.131199 | 1.891654 |
| (SEQ ID NO: 864) | | |
| C64 | 0.564561 | 1.110898 |
| (SEQ ID NO: 871) | | |
| C18 | 0.85826 | 1.041825 |
| (SEQ ID NO: 874) | | |
| C19 | 1.607973 | 0.910107 |
| (SEQ ID NO: 876) | | |
| C65 | 0.733764 | 2.255002 |
| (SEQ ID NO: 922) | | |
| C20 | 0.695179 | 1.435898 |
| (SEQ ID NO: 959) | | |
| C21 | 1.145246 | 0.792179 |
| (SEQ ID NO: 983) | | |
| C22 | 0.789058 | 0.666628 |
| (SEQ ID NO: 984) | | |
| C23 | 0.740537 | 1.420103 |
| (SEQ ID NO: 1015) | | |
| C24 | 1.450209 | 0.95303 |
| (SEQ ID NO: 1017) | | |
| C25 | 1.284727 | 0.659544 |
| (SEQ ID NO: 1023) | | |
| C26 | 0.541319 | 0.730967 |
| (SEQ ID NO: 1026) | | |
| C92 | 0.00527 | 1.511334 |
| (SEQ ID NO: 1037) | | |
| C28 | 0.548442 | 1.278064 |
| (SEQ ID NO: 1041) | | |
| C29 | 0.925584 | 1.340266 |
| (SEQ ID NO: 1047) | | |
| C30 | 0.250089 | 0.888613 |
| (SEQ ID NO: 1059) | | |
| C31 | 0.775688 | 2.897512 |
| (SEQ ID NO: 1068) | | |
| C32 | 0.63106 | 0.733483 |
| (SEQ ID NO: 1134) | | |
| C33 | 1.046387 | 0.208604 |
| (SEQ ID NO: 1168) | | |
| C34 | 1.34302 | 1.69471 |
| (SEQ ID NO: 1169) | | |
| C35 | 1.369419 | 2.275963 |
| (SEQ ID NO: 1171) | | |
| C36 | 0.75288 | 1.034014 |
| (SEQ ID NO: 1177) | | |
| C37 | 0.510432 | 0.79276 |
| (SEQ ID NO: 1178) | | |
| C38 | 1.167913 | 0.700155 |
| (SEQ ID NO: 1179) | | |
| C39 | 0.9501 | 1.236702 |
| (SEQ ID NO: 1180) | | |
| C40 | 0.855226 | 2.311516 |
| (SEQ ID NO: 1189) | | |
| C41 | 1.360869 | 2.100648 |
| (SEQ ID NO: 1192) | | |
| C42 | 0.960246 | 0.557141 |
| (SEQ ID NO: 1193) | | |
| C43 | 0.881406 | 1.044061 |
| (SEQ ID NO: 1198) | | |
| C44 | 1.840964 | 1.301044 |
| (SEQ ID NO: 1216) | | |
| C45 | 0.469295 | 0.806161 |
| (SEQ ID NO: 1263) | | |
| C46 | 0.568126 | 1.365778 |
| (SEQ ID NO: 1276) | | |
| C47 | 0.632638 | 0.991396 |
| (SEQ ID NO: 1280) | | |
| C48 | 0.375273 | 1.474358 |
| (SEQ ID NO: 1282) | | |
| C49 | 1.264343 | 0.887098 |
| (SEQ ID NO: 1284) | | |
| C57 | 1.152769 | 1.55509 |
| (SEQ ID NO: 2285) | | |
| C93 | 0.503822 | 1.576826 |
| (SEQ ID NO: 75) | | |
| C94 | 0.526345 | 2.45618 |
| (SEQ ID NO: 77) | | |
| C58 | 0.0014 | 0.003517 |
| (SEQ ID NO: 2601) | | |
| C59 | 0.051441 | 0.00131 |
| (SEQ ID NO: 2615) | | |
| C60 | 0.008382 | 0 |
| (SEQ ID NO: 2616) | | |
| C61 | 0.007267 | 0.006067 |
| (SEQ ID NO: 2617) | | |
| C62 | 0.008072 | 0.00268 |
| (SEQ ID NO: 2618) | | |
| C63 | 0.000761 | 0.040465 |
| (SEQ ID NO: 2627) | | |
| C83 | 0.004556 | 0.002569 |
| (SEQ ID NO: 137) | | |
| C87 | 0.059416 | 0.133348 |
| (SEQ ID NO: 532) | | |
| C88 | 0.036736 | 0.168426 |
| (SEQ ID NO: 566) | | |
| C89 | 0.760741 | 0.697294 |
| (SEQ ID NO: 580) | | |
| C90 | 0.983704 | 2.273875 |
| (SEQ ID NO: 648) | | |
| C1 | 0.781812 | 0.102251 |
| (SEQ ID NO: 693) | | |
| C2 | 0.843272 | 2.085294 |
| (SEQ ID NO: 752) | | |
| C3 | 1.084859 | 0.915046 |
| (SEQ ID NO: 785) | | |
| C4 | 0.875589 | 0.920996 |
| (SEQ ID NO: 787) | | |
| IRC1-947 | 0.702179 | 1.6859 |
| (SEQ ID NO: 791) | | |
| C5 | 1.530501 | 1.885392 |
| (SEQ ID NO: 793) | | |
| C7 | 0.669549 | 1.033134 |
| (SEQ ID NO: 820) | | |
| C8 | 1.350993 | 2.682581 |
| (SEQ ID NO: 823) | | |
| C9 | 0.823706 | 0.94879 |
| (SEQ ID NO: 839) | | |
| C10 | 1.101436 | 1.308508 |
| (SEQ ID NO: 840) | | |
| C11 | 0.69405 | 1.890697 |
| (SEQ ID NO: 841) | | |
| C66 | 0.016487 | 0.09984 |
| (SEQ ID NO: 2667) | | |
| C67 | 0.058548 | 0.213187 |
| (SEQ ID NO: 2681) | | |
| C68 | 1.088459 | 1.130929 |
| (SEQ ID NO: 2742) | | |
| C69 | 0.587669 | 1.383575 |
| (SEQ ID NO: 2746) | | |
| C70 | 0.325456 | 0.78795 |
| (SEQ ID NO: 2758) | | |
| C71 | 1.050785 | 1.64216 |
| (SEQ ID NO: 2777) | | |
| C72 | 1.350472 | 0.19906 |
| (SEQ ID NO: 2778) | | |
| C84 | 0.546897 | 1.306713 |
| (SEQ ID NO: 3289) | | |
| C85 | 1.191477 | 1.458442 |
| (SEQ ID NO: 3290) | | |
| C95 | 2.538039 | 3.547121 |
| (SEQ ID NO: 3288) | | |
| C51 | 0.601175 | 1.176565 |
| (SEQ ID NO: 3292) | | |
| C52 | 1.022958 | 2.268061 |
| (SEQ ID NO: 3293) | | |
| C53 | 0.955833 | 1.218078 |
| (SEQ ID NO: 3294) | | |
| C54 | 0.718225 | 1.145427 |
| (SEQ ID NO: 3295) | | |
| C55 | 0.092929 | 0.047994 |
| (SEQ ID NO: 3296) | | |
| C56 | 0.665661 | 0.970387 |
| (SEQ ID NO: 3297) | | |
| C73 | 0.857241 | 2.009902 |
| (SEQ ID NO: 3298) | | |
| C74 | 0.445517 | 0.65993 |
| (SEQ ID NO: 3299) | | |
| C75 | 0.000962 | 0.903645 |
| (SEQ ID NO: 3300) | | |
| C76 | 0.479121 | 1.038262 |
| (SEQ ID NO: 3301) | | |
| C77 | 1.01908 | 1.176483 |
| (SEQ ID NO: 3302) | | |
| C79 | 0.621993 | 2.738178 |
| (SEQ ID NO: 3284) | | |
| C80 | 0.604474 | 2.219763 |
| (SEQ ID NO: 3285) | | |

### Example 87

### IRES Screening

Approximately 12,400 potential internal ribosome entry sites (IRES) sequence candidates were computationally identified from untranslated regions (UTRs), e.g., viral UTRs, in partial and complete publicly-available viral sequences available (e.g., Genbank). Sequences lacking a Xbal site and less than a certain number of homopolymers were removed as potential candidates. The remaining 8,077 sequence candidates were attached to the 3′ end of a unique barcode sequence. Barcode sequences were developed using batch barcode sequences and by removing the Xbal site along with the last 2 nucleotides of the linker sequence. Additional 107 IRES candidate were selected from known IRES sequences (e.g., IRES Base). These additional IRES candidates were narrowed using the same parameters as the UTR-derived IRES sequences and added to the 3′ end of a unique barcode sequence. As a control, five IRES sequences, SEQ ID NOs: 3282-3285 and 3288, and were repeated eight times, and each attached to the 3′ end of a unique barcode sequence. Redundant sequences were removed. “caaaacaaacacaaaccaacaaaaa” (SEQ ID NO: 24897) and “ATGGGAGTGAAGGTGCTGTTCGCCC” (SEQ ID NO: 24898) were added to the 5′ and 3′ ends of all the sequences. Any sequences that acquired an additional Xbal site were removed. 1,0763 sequences remained and were analyzed.

### Functional Screening in T Cell Culture

Each DNA template is cloned to include an IRES sequence selected from a sequence set forth in in any one of SEQ ID NOS: 14067-24829 (GIRES-1 through GIRES-10762) or a fragment thereof. In the constructs set forth herein, each IRES sequence of SEQ ID NOs: 14067-24829 is optionally barcoded with one barcode sequence of SEQ ID NOs: 3304-14066 (e.g., the IRES of SEQ ID NO: 14067 is barcoded with the barcode sequence of SEQ ID NO: 3304, the IRES of SEQ ID NO: 14068 is barcoded with the barcode sequence of SEQ ID NO:3305, the IRES of SEQ ID NO: 14069 is barcoded with the barcode sequence of SEQ ID NO: 3306, etc.). Exemplary constructs include a 5′ enhanced intron element, a 5′ enhanced exon element, a putative IRES, a coding element encoding Gaussia Luciferase, a 3′ enhanced exon element, and a 3′ enhanced intron element. Engineered circular RNA (oRNA) are generated from the DNA template by in vitro transcription (IVT), and oRNA are subsequently purified from non-circular RNA components of the IVT reaction. Illumina sequencing and polysome profiling are performed after delivering the oRNA to primary human T cell culture. Time-course experiments on total and ribosome fractions after delivering the oRNA to primary human T cell culture, and validation experiments are also performed on the oRNA constructs.

oRNA-LNP Formulations

Purified oRNA is nanoprecipitated with lipids to form LNP-oRNA formulations. LNPs are formulated first by creating empty lipid nanoparticles using a Precision Nanosystems Ignite instrument with a “NextGen” mixing chamber. Ethanol phase contains Lipid 39 from Table 2, DSPC, cholesterol, and DMG-PEG 2000 (Avanti Polar Lipids, Inc., Alabaster, AL) at a molar ratio of 45:9:44:2 are combined with 50 mM sodium acetate buffer at pH 4.5. A 3:1 aqueous:ethanol mixing ratio is used. The empty LNPs are dialyzed in 1 L of 1×PBS and exchanged 2 times over 18 hours. Dialyzed empty LNPs are then mixed with a final circular RNA concentration of 40 g/mL in 50 mM sodium acetate buffer at pH 4.5. A 3:1 aqueous:empty lipid nanoparticle mixing ratio is used. The LNPs final ionizable lipid:RNA phosphate ratio is at 6.0.

### IRES Screening In Vivo

oRNA-LNP constructs are delivered to mice or non-human primates. Polysome profiling and time course experiments are performed for target organs and tissues. Further validation experiments are conducted in the respective animal models.

### Example 88

### IRES Library Cloning

Naturally occurring and optionally truncated untranslated region (UTR) sequences were selected as internal ribosome entry sites (IRES) candidates. 10,304 IRES sequences were synthesized by a gene synthesis vendor in 11 pools (each pool comprised of approximately 701-999 sequences per pool). IRES sequences received one out of 8 different barcode sequences. To clone the TRES sequences, a plasmid backbone was designed to comprise a 5′ enhanced intron element, a 5′ enhanced exon element, a Gaussia luciferase expression sequence, a 3′ enhanced exon element, and a 3′ enhanced intron element along with a 5′ primer (e.g., ATGGGAGTG AAGGTGCTGTTCGC, SEQ ID NO: 24899) and a 3′ primer (e.g., TTTTTGTTGGTTTGTGTTTGTTTTGTT TGGTGTTTGC, SEQ ID NO: 24900). The 5′ and 3′ enhanced intron and exon elements of the plasmid backbone were derived from Anabaena. The plasmid backbones were then amplified, and the original backbone attached to the IRES sequences were digested using Dpnl.

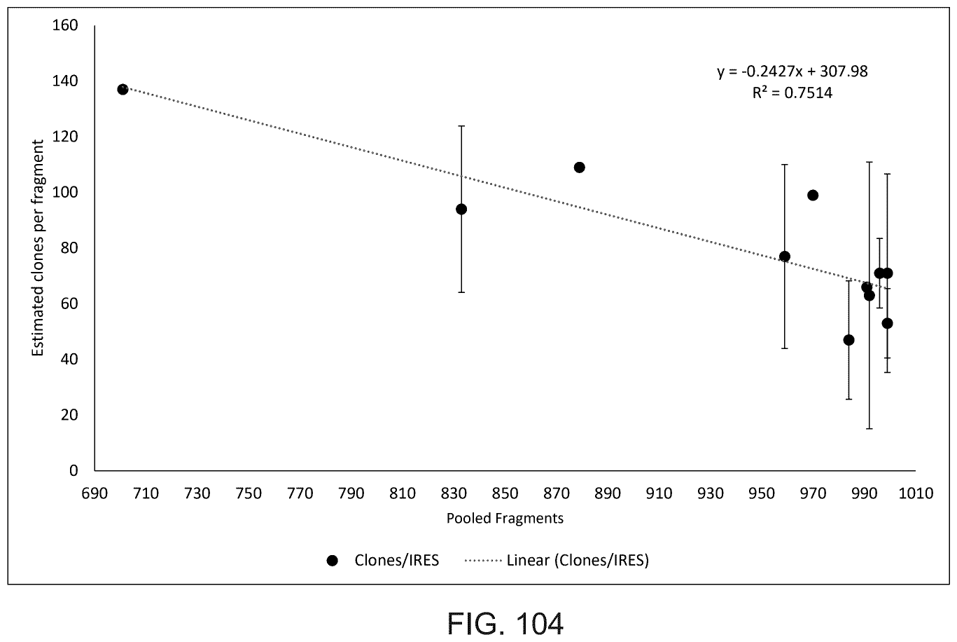

Each pool was cloned individually by combining the plasmid backbone and the IRES sequence at a 1:2 ratio, respectively, and incubated with a Gibson Assembly HiFi master mix at 50° C. for 30 minutes. 2 μL of the solution was transformed into 5-alpha competent cells. Transformants were pooled and grown in 1 L LB-ampicillin overnight and plasmid was isolated using Qiagen MaxiPrep kit. Estimated clones per fragment were determined by back calculating from the number of bacterial colonies. FIG. 104 shows the estimated number of clones produced per TRES for the number of sequences contained in each of the 11 pools.

Generating Circular RNAs from a IRES Cloning Library

Circular RNA was generated from the pooled plasmid output of the TRES cloning library. The plasmids of the pooled plasmid output was linearized with XbaI, and incubated with NO NTPs, GMP, in vitro transcription (IVT) buffer, RNase Inhibitor, Pyrophosphatase, and T7 polymerase for 2 hours at 37° C. in a thermomixer shaking at 350 RPM. DNaseI was then added to the reaction mix and incubated for 15 minutes at 37° C. Triplicates of resulting IVT product were purified using Monarch RNA purification column to conduct an IRES circularization assay. For enrichment of the circularized product, each replicate of the IVT product was subjected to exonuclease treatment of 15 μg of cleaned IVT product was incubated with RNase R buffer (Fisher Scientific), 10 U of RNase R (Fisher Scientific), 1U of XRN-1 (New England Biolabs) for 45 min at 37° C. Circular RNA enriched product was purified using Monarch RNA purification column.

For polysome and stability profiling, the IVT products were then diluted with 2.5× volumes of water with 3.5× volumes of dT resin binding buffer and incubated at 55° C. for 10 minutes. The IVT products in dT resin binding buffer are then bound to 2.5× volumes equilibrated Poros dT resin, and then incubated at 70° C. for 15 minutes at 700 RPM of shaking. Supernatant was then spun through the resin and purified using Monarch RNA purification column using manufacturer specifications.

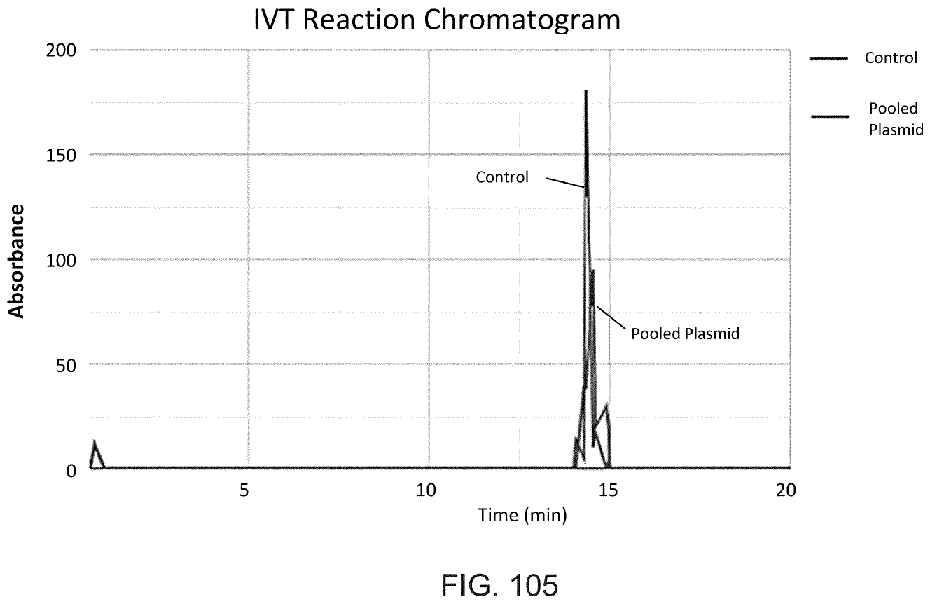

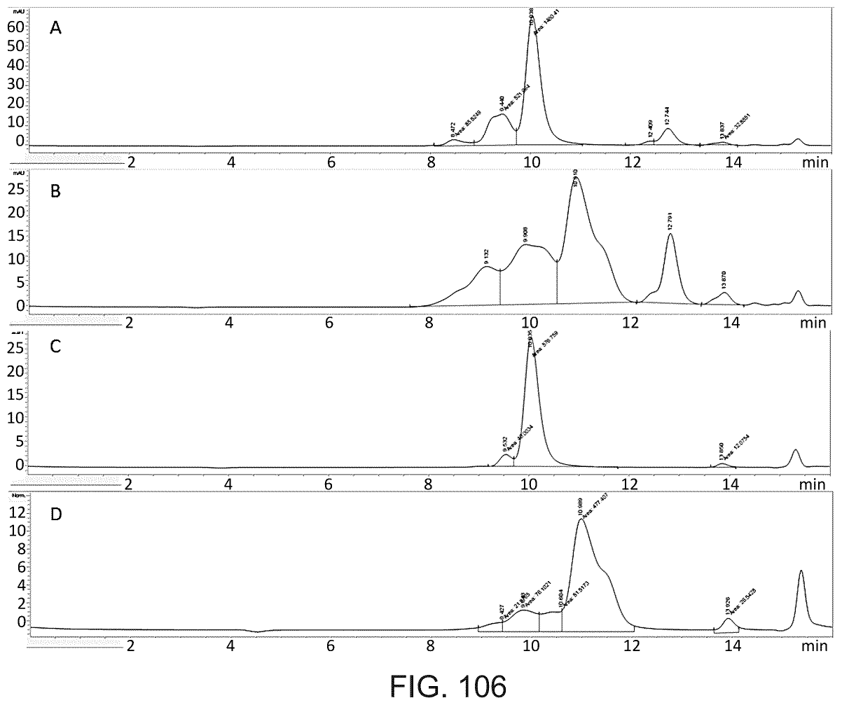

A circular RNA ladder was generated by cloning 8 scrambled IRES sequences to the same plasmid backbone as the IRES library. For circularization profiling, the IVT products of these plasmids were mixed at defined concentrations across 4 orders of magnitude. The IVT products spiked-in to the IRES IVT product and were subjected to exonuclease treatment with the TRES IVT product. For polysome and stability profiling experiments, the IVT products of these ladders were cleaned up using dT resin and the purification column as described above. The purified products were mixed at defined concentrations across 4 orders of magnitude. FIG. 105 depicts the IVT reaction of the pooled plasmid output. FIG. 106 illustrates the IVT reaction of the pooled plasmid output before and after RNase R digestion.

### Example 89

### Cytoplasmic Polysome Profiling and Total RNA Isolation of Circular RNA Comprising IRES

a. T Cell Electroporation

Human pan-T cells were grown with Advanced RPMI media, supplemented with Normocin, 10% FBS, Penicillin and Streptomycin, and GlutaMAX. T-cells were activated after thawing by adding CD3 and CD34 for 3 days, and then replaced with media containing IL-2. Cells were expanded in this media, keeping density under 3×10⁶/ml, to appropriate cell numbers. Electroporation was performed using the 4D Nucleofector X unit and P3 primary cell 4D Nucleofector X kit L. Cells were resuspended in P3 solution containing 50 μg IRES library/mL at a density of 5×10⁷ cells/mL. 100 μL of the resuspended solution was transferred to a cuvette and electroporated. Cells were then resuspended in 5 mL T cell media per 100 μL electroporated cells (density of 1×10⁶ cells/mL) and placed in a growth container.

b. T-Cell Collection

60M of the resuspended cells were electroporated for polysome experiments, and 30M of the resuspended cells were electroporated for stability experiments. Polysome collection was performed by taking all cells in the growth container at 6 hours post electroporation and adding cycloheximide at 100 μg/ml. These cells were then incubated at 37° C. for 15 minutes and received 2 washes with PBS+ cycloheximide at 100 μg/ml per wash. After removal of the final wash (i.e., the second wash), the cells were flash frozen on dry ice and stored at −80° C. The same process was performed for 4 different time points (e.g., 1.5 hours, 6 hours, 24 hours, and 48 hours) with a portion of the cell pool removed to a conical tube and treated with cycloheximide.

c. Polysome Profiling and RNA Isolation

Cell pellets were thawed briefly on ice, and then resuspended in polysome isolation buffer (e.g., 25 mM Tris-HCl pH 7.5, 100 mM KCl, 5 mM MgCl₂, 1% IGEPAL-CA630, 1% Sodium Deoxycholate, 100 μg/ml Cycloheximide, 1 mM DTT, 60 U/ml Superase Inhibitor, 1× complete EDTA free Protease Inhibitor). The solution was then incubated on ice for 30 minutes. After 30 minutes, the debris was spun down at 21,000×g, 10 minutes, 4° C. For the stability experiment, RNA extraction was directly performed. Trizol LS was added at 4× volume, and 100% Ethanol was added at 5× volume, then RNA extraction was performed using Zymo Direct-zol magbead RNA kit using manufacturer's recommendations.

For polysome experiments, 10-50% sucrose gradients were created using guidelines and standard protocols from a Biocomp Gradient Master instrument. Gradients were layered with the RNA extracts and centrifuged using Beckman XE-90 ultracentrifuge and SW-41Ti rotor at 41,000 RPM for 1.5 hours. These gradients were then fractionated using Biocomp Piston Gradient Fractionator and Gilson Fraction collector, where it collected 250 μL fractions along the gradient.

The circRNA ladder with scrambled IRES sequences was then spiked into each fraction at 2% v/v, and RNA was extracted from each fraction as described in Example 88.

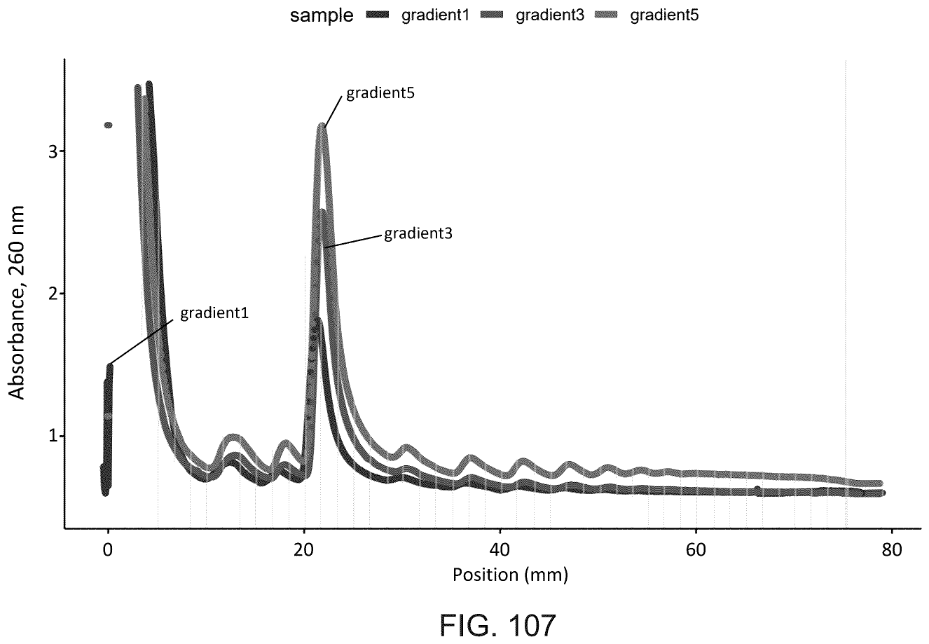

FIG. 107 provides the 260 nm absorbance profile of some of the polysome species contained in several of the sucrose gradients following centrifugation.

### D. Total RNA-Seq Library Generation

For the circularization profiling, IVT and exonuclease treated RNA samples were made to RNA-seq libraries using NEBNext Ultra II Directional RNA-seq library kit and NEBNext Multiplex Oligos for Illumina with Unique Dual Index for RNA following their manuals. Libraries were then pooled in equimolar amounts and sequenced using Illumina Nextseq 2000, P2 kit reagents.

### E. circRNA Library Generation

For the stability profiling, total RNA from each timepoint and replicate was spiked in at 0.01% concentration, and measured by RNA concentration, using the scrambled circular RNA ladder described above. For the polysome profiling, all fractions corresponding to RNA bound to 4 or more ribosomes were pooled equally (henceforth “high” library), and all fractions corresponding to RNA bound to less than 4 ribosomes were pooled equally into a separate pool (henceforth “low” library). For each of these polysome pools and stability samples, RNA integrity was measured using Tape Station to validate that the RNA Integrity score was 9 or greater. These RNA samples were then depleted of rRNA using Ribo Minus rRNA depletion kit, using the low input or high input protocol depending on RNA amount. The output was then treated with 5 Units RNase R from Lucigen for 15 minutes at 37° C. The resulting RNA was purified using Beckman RNAclean XP beads. RNA libraries were prepared using NEBNext Ultra II Directional RNA-seq library kit and NEBNext Multiplex Oligos for Illumina with Unique Dual Index for RNA using the pre-enriched protocol. Libraries were then pooled in equimolar amounts and sequenced using Illumina Nextseq 2000, P3 kit reagents.

f. Bioinformatic Analysis of the IRES Library

Each circRNA within the IRES library and scrambled spike in was quantified by a custom RNA-seq protocol. Sequence reads were monitored for quality, and low-quality reads were trimmed using cutadapt. The sequence readings were then mapped to the library reference using BWA, and UMIs. Next, the sequence readings were grouped, called for consensus, and filtered for PCR duplicates and low-quality mappings using fgbio tools. The resultant files were quantified using Kallisto. Kallisto quantities were compiled into a counts table for each of the profiling experiments.

For the circularization experiment, the enrichment of each construct in each replicate was calculated by contrasting the count in the exonuclease treated library to the IVT library after filtering for a minimal count after library size normalization.

For polysome and stability profiling experiments, each count was normalized using linear regression for spiked-in counts and quantities, then filtered for a minimal count after normalization.

For polysome profiling experiment, proportion high values were calculated as follows:

$${Proportion}_{high} = \frac{{count}_{high}}{{count}_{high} + {count}_{low}}$$

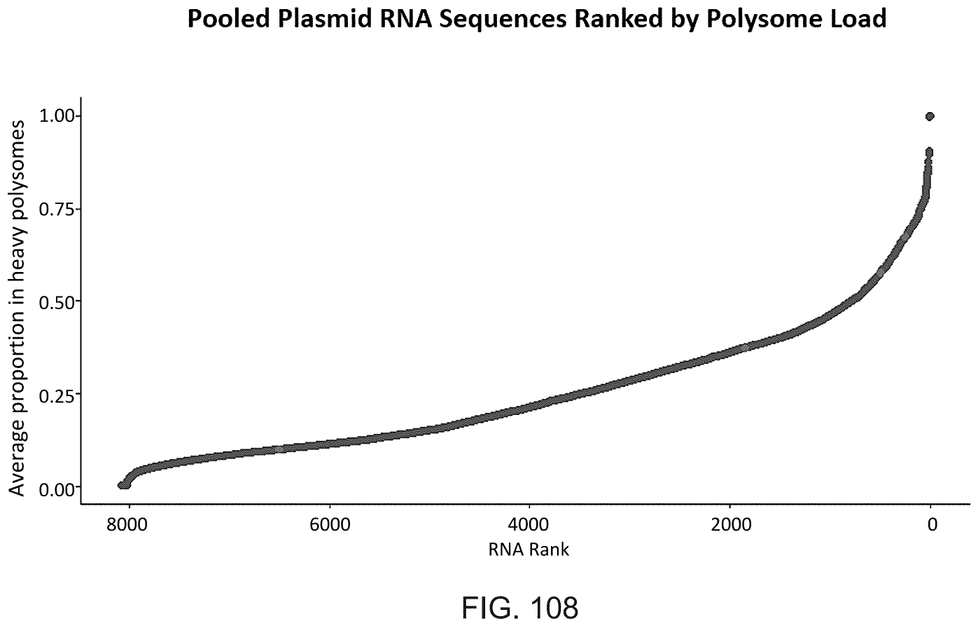

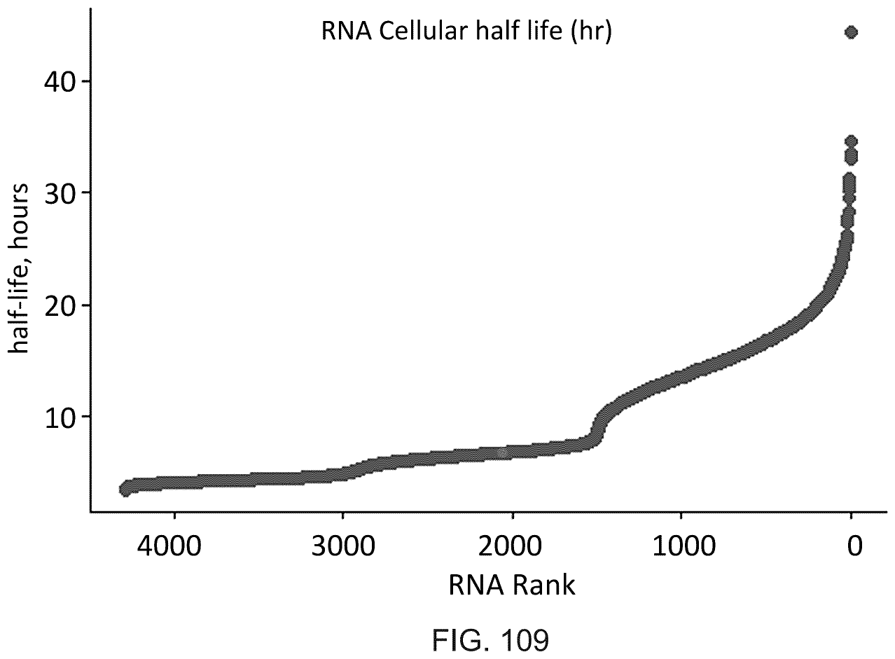

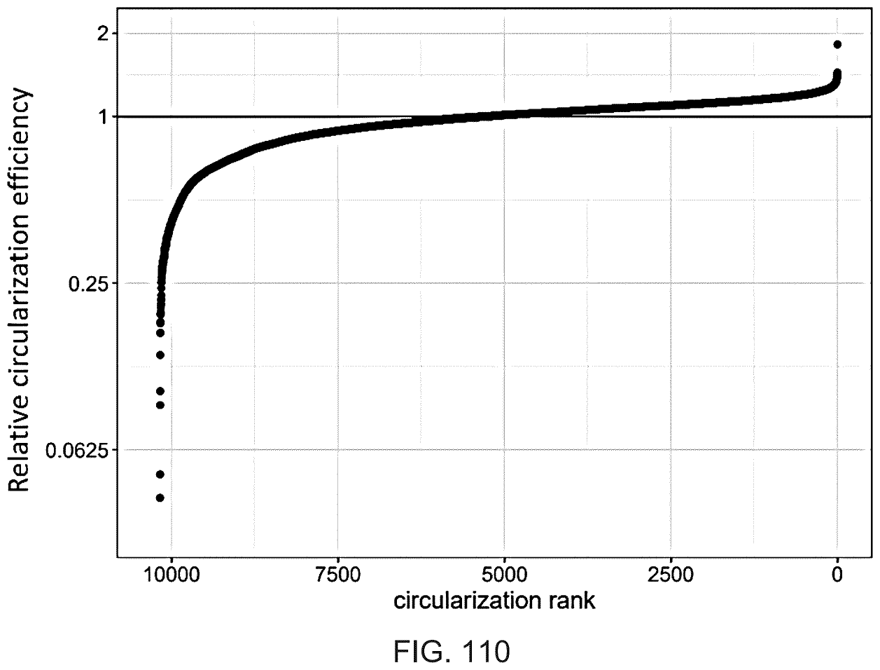

In the stability experiment, after normalization constructs were filtered for having a minimal count in at least 3 timepoints. These values were then normalized to the first time point being 1, and natural log transformed. Each remaining construct then had linear regression performed, and high confidence linear regressions were selected by filtering for a p-value of 0.05 on the regression and an R² value of >0.6. Half-life was then calculated using the following formula:

$${t}_{1 / 2} = \frac{ln ( 10 ) - b}{a} - \frac{ln ( 20 ) - b}{a}$$

Where a is the coefficient of the linear regression and b is the intercept of the linear regression. FIGS. 108-110 provides the average portion in heavy polysomes, estimated half-life of the circular RNAs, and relative circularization efficiency.

### Example 90

Expression from Circular RNA Constructs In Vitro

Circular RNA constructs were engineered from vectors to express dystrophin protein. Vector constructs used comprised a 5′ enhanced intron element, a 5′ enhanced exon element, an IRES, an expression sequence encoding for a dystrophin protein comprising approximately 2,014 amino acids, a 3′ enhanced intron element, and a 3′ enhanced exon element. The IRES of the circular and vector constructs comprised of sequences from Tables 5 and 6 [SEQ ID NO: 3283, SEQ ID NO: 3282, SEQ ID NO: 1284, SEQ ID NO: 1177, SEQ IDNO: 2285, SEQ IDNO: 3287, SEQ IDNO: 3291, SEQ IDNO: 24893, SEQ IDNO: 24894]. The 5′ and 3′ enhanced intron elements comprised a sequence from Anabaena intron. The vector and circular RNA constructs further comprised an V5 antibody tag sequence upstream to the expression sequence but downstream to the IRES.

### Myoblast Culture

C2C12 (ATCC CRL-1772) myoblast cultures were thawed in a 37° C. water bath and plated at recommended seeding density (5,000 per cm²) in growth media consisting of DMEM, high glucose (Gibco) supplemented with 10% fetal bovine serum (FBS) (R&D Systems) and 2% Penicillin Streptomycin (PSG) (Gibco) and allowed to grow in a tissue culture incubator at 37° C. and 5% CO₂ atmosphere. If necessary to subculture, the cells were detached using TrypLE Express (Gibco) and replated in growth media. Media was changed every 2 days.

### Myotube Differentiation

C2C12 myoblasts were grown to 70-80% confluency in 0.1% gelatin (Sigma) coated tissue culture plates. Once at 70-80% confluency growth media was removed, cells were washed twice in 1×PBS (Gibco) and media was changed to differentiation media consisting of dulbecco's Modified Eagle Medium (DMEM) (Gibco) supplemented with 2% horse serum (Gibco) and 2% Penicillin-Streptomycin (PSG) (Gibco). Media was changed daily for 6-7 days until nearly all myoblasts had differentiated into myotubes.

Transfection of the Circular RNAs into the Myotube Cells

For each 96-well plate reaction, 0.3 μL of Lipofectamine-3000 transfection reagent (Lipo3K) (ThermoFisher) was mixed with 5 μL Opti-MEM reduced serum media (ThermoFisher). In a separate tube, per reaction, circular RNA encoding firefly luciferase (fluc) (at 10-200 ng) was combined with 5 μL Opti-MEM and 0.2 μL P3000TM enhancer reagent. 5 μL of Lipo3K/Opti-MEM was then added to 5 μL of circular RNA/Opti-MEM and incubated at room temperature for 15 mins. 10 μL of Lipo3000/circRNA mixture was then added to each well to be transfected and placed in a tissue culture incubator at 37° C. and 5% CO² atmosphere for 24-hrs (for luciferase assay readout) or 48-hours (for immunoblotting readout).

### LNP Transfection

Once myotubes were fully formed and ready for transfection, new differentiation media was added (e.g., 1 mL per 12-well plate), formulated LNPs comprising ionizable lipid 123 of Table of Additional Exemplary Ionizable Lipids (Table B) were then added directly into the differentiation media and cells were placed in a tissue culture incubator at 37° C. and 5% CO₂ atmosphere for 48-hours prior to collection.

### Cell Collection

48-hours post transfection differentiation media was removed and cells were washed twice with 1×PBS (Gibco), 500 ul of trypsin (ReagentPack Lonza) were added and kept at room temperature until cells detach. The trypsin was then quenched with 700 μL of trypsin neutralizing solution (ReagentPack Lonza). The cells then were collected and spun down at 220×g for five minutes. The supernatant was removed, and cells were resuspended in RIPA buffer (Thermo) supplemented with proteinase inhibitor cocktail (Complete Sigma). Cells were vortexed and allowed to sit on ice for about 20 mins and then spun down at 10,000×g for 10 minutes, supernatant is protein lysate.

### Bradford Assay

To quantify protein concentrations 5 μL of pre-diluted bovine serum albumin (BSA) standards (Thermo) were loaded onto a 96-well plate in triplicate. Protein lysates were loaded at 1 ul in triplicate as well. Room temperature Quick Start Bradford Dye (BioRad) was added at 200 μL per well and allowed to sit for at least 5 mins. Absorbance was read at 595 nm using Cytation 5 plate reader (Bio-tek). Concentrations were calculated based on a standard curve.

### Western Blot Analysis

For the western blot analysis, protein lysate samples (cell or tissue) were made up at 1 μg/μL using NuPAGE 4× sample buffer (Thermo) and 1× NuPAGE reducing agent (Thermo). Samples were then heated at 95° C. for 8 minutes and spun down before loading. Samples were then loaded onto a3-8% Tris-Acetate gel (Thermo), placed in a XCell SureLock Mini-Cell Module (Thermo) and run at 80V for 10 mins, the module was then placed on ice and run at 120V for 80 mins. Proteins were then transferred to a nitrocellulose membrane (Thermo) using a wet transfer system (BioRad) in the cold room for 2 hrs at 80V. Membranes were then taken out and blocked in Intercept blocking buffer (LICOR) for 1 hr and then placed in diluted in Intercept T20 antibody diluent (LICOR) overnight at 4° C. The next day, the membranes were washed 3 times for 5 mins in 1× TBST, secondary antibody was then added at 1:5000 for 1 hr at room temperature. The membranes were then washed 3×5 mins in 1× TBST and imaged using Odyssey CtX.

### Immunoprecipitation

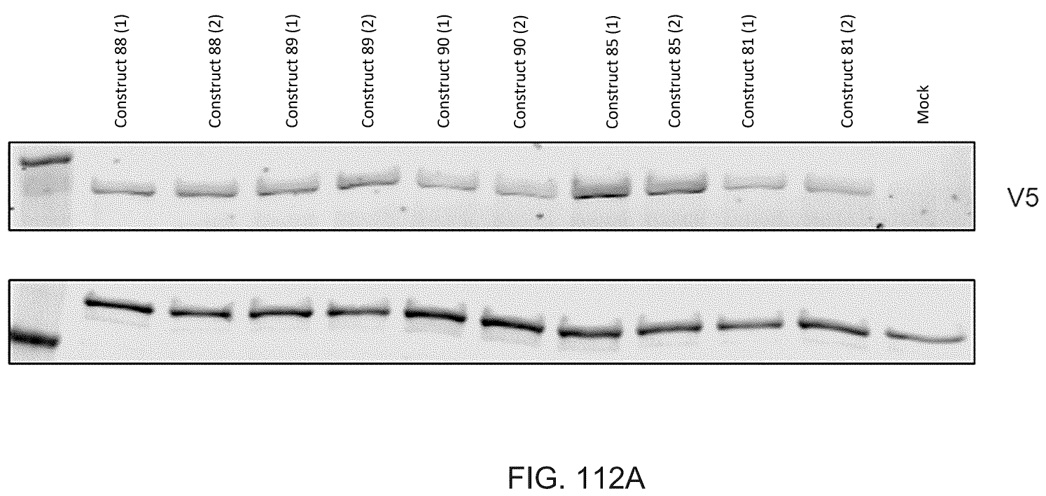

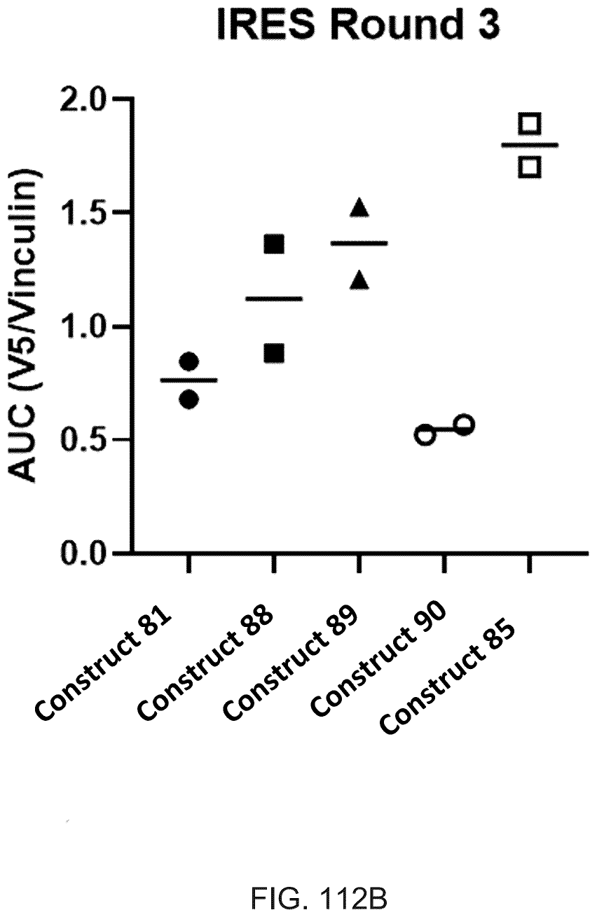

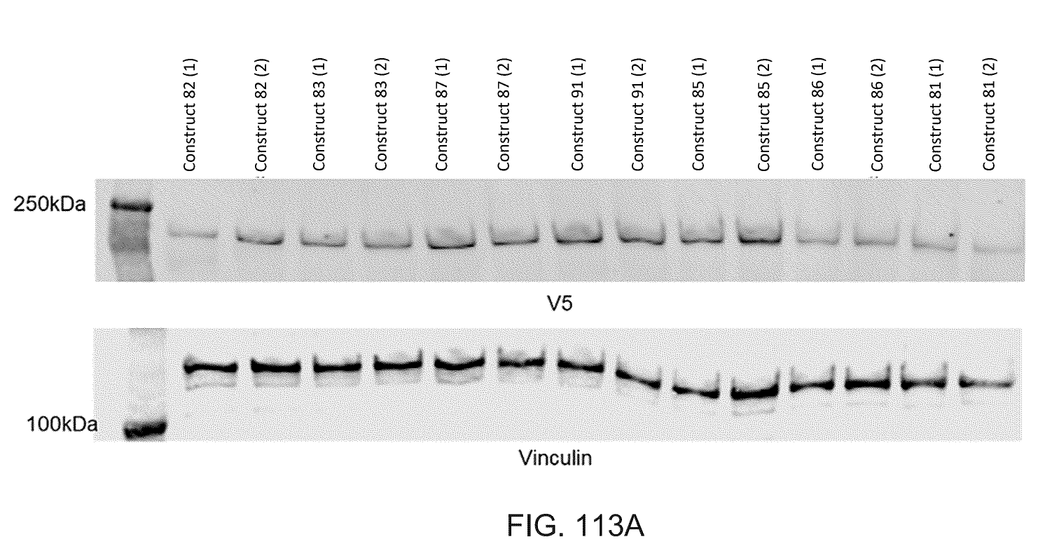

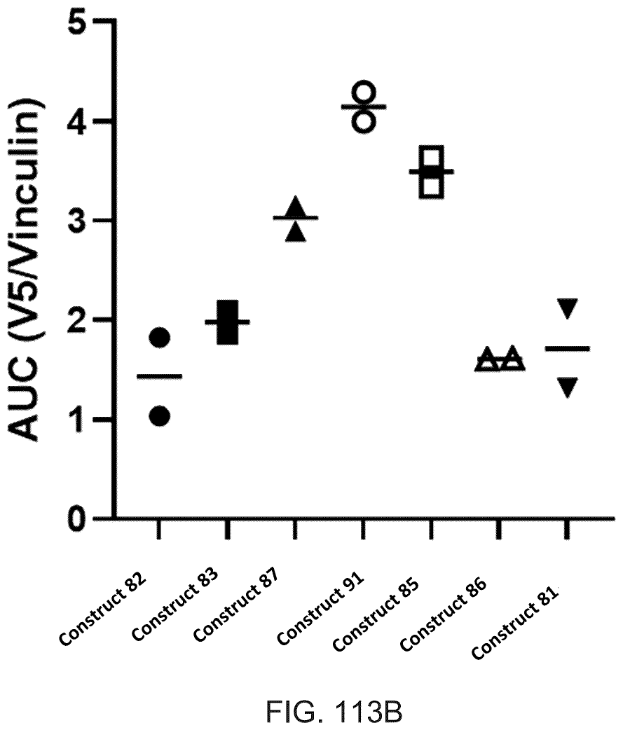

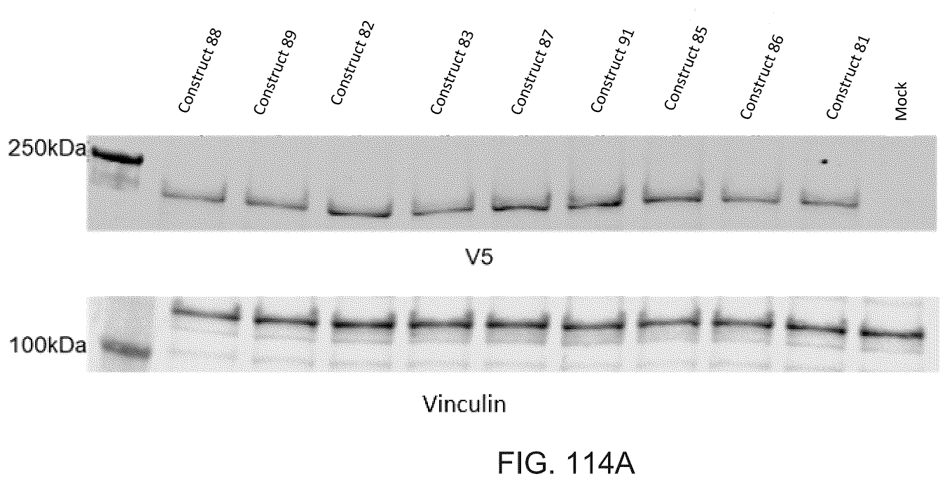

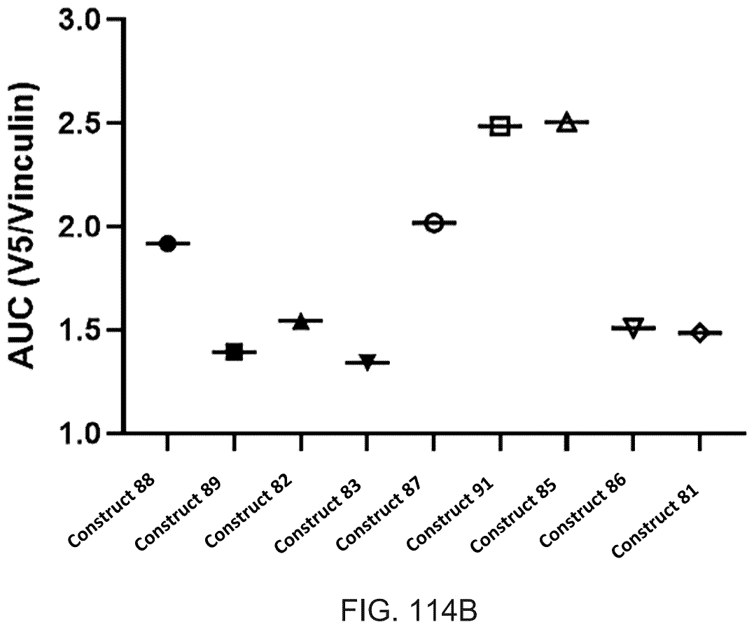

Immunoprecipitation of V5-tag was carried out by first adding 7.5 μg of anti-V5-tag antibody (abcam) to 1-1.5 mg of protein lysate (250 μL total volume) and incubating overnight at 4° C. 100 μL of Pierce protein A/G magnetic agarose beads (Thermo) were magnetized using DynaMag-2 magnet (Invitrogen) and washed twice with 1×PBS-0.1% triton. Lysate-antibody mix was added to the magnetized beads, mixed gently and incubated at 4° C. for 3 hours. Beads were then magnetized and supernatant discarded, beads were washed 5× with 700 μL of 1×PBS-0.1% triton and magnetized again. 20 μL of 4× NuPAGE sample buffer and 1× NuPAGE reducing agent (Thermo) was added to re-suspend the beads, heated to 90° C. for 7 minutes and vortexed vigorously to detach protein from bead. Beads were magnetized and supernatant moved to a clean tube to later run on a western blot as described above. Blot was probed for dystrophin using a rabbit anti-dystrophin antibody (abcam). FIGS. 111-114 provide the immunoprecipitation results.

TABLE 10

| Construct | | | % | AUC/% | Norm to |
|---|---|---|---|---|---|
| # | IRES Sequence | RNA | splice | splice | 10.0000% |
| 81 | SEQ ID NO: 3282 | 10.0006 | 72.5 | 1.19 | 100 |
| 82 | SEQ ID NO: 1177 | 07.0074 | 49.7 | 3.04 | 255 |
| 83 | SEQ ID NO: 2285 | 07.0075 | 53.3 | 2.47 | 207 |
| 84 | SEQ ID NO: 3287 | 07.0076 | 55.2 | 2.48 | 209 |
| 85 | SEQ ID NO: 3291 | 07.0077 | 60.4 | 4.43 | 372 |
| 86 | SEQ I NO: 24893 | 07.0078 | 42.8 | 4.43 | 372 |
| 87 | SEQ ID NO: 24894 | 07.0079 | 61.2 | 3.06 | 257 |

TABLE 11

| Construct | | | % | AUC/% | Norm |
|---|---|---|---|---|---|
| # | IRES Sequence | RNA | splice | splice | to 100.000% |
| 88 | SEQ ID NO: 3283 | 07.0070 | 0.62 | 1.81 | 172 |
| 89 | SEQ ID NO: 1284 | 12.0022 | 0.58 | 2.35 | 224 |
| 90 | SEQ ID NO: 3282 | 12.0023 | 0.43 | 1.26 | 121 |
| 85 | SEQ ID NO: 3291 | 12.0024 | 0.66 | 2.72 | 259 |
| 81 | SEQ ID NO: 3282 | 10.0006 | 0.725 | 1.05 | 100 |

TABLE 12

| | | | | | | | Construct | AUC/ | (AUC/splice)/ |
|---|---|---|---|---|---|---|---|---|---|
| Construct | IRES | | Splice | | AUC/splice | Construct | 81/% | Construct | Construct 81 |
| # | Sequence | RNA | percent | AUC | perc. | 81 | splice | 81 (%) | (%) |
| 82 | SEQ ID | 7.0074 | 0.5 | 1.543 | 2.86 | 1.71 | 2.377 | 83 | 120 |
| | NO: 1177 | | | | | | | | |
| 83 | SEQ ID | 7.0075 | 0.53 | 1.98 | 3.73 | 1.71 | 2.377 | 115 | 157 |
| | NO: 2285 | | | | | | | | |
| 85 | SEQ ID | 7.0077 | 0.6 | 3.49 | 5.81 | 1.71 | 2.377 | 204 | 245 |
| | NO: 3291 | | | | | | | | |
| 86 | SEQ I NO: | 7.0078 | 0.43 | 1.607 | 3.73 | 1.71 | 2.377 | 94 | 157 |
| | 24893 | | | | | | | | |
| 87 | SEQ ID | 7.0079 | 0.61 | 3.02 | 4.96 | 1.71 | 2.377 | 177 | 209 |
| | NO: 24894 | | | | | | | | |
| 91 | SEQ ID | 8.0041 | 0.76 | 4.14 | 5.45 | 1.71 | 2.377 | 242 | 230 |
| | NO: | | | | | | | | |
| | 24895 | | | | | | | | |

### Example 91

Muscular Dystrophy Expressed from Circular RNA Construct Having Various IRESes In Vivo

Circular RNA constructs were engineered from vectors to be able to express a dystrophin protein. Vector constructs used comprised a 5′ enhanced intron element, a 5′ enhanced exon element, an IRES, an expression sequence encoding for a dystrophin protein, a 3′ enhanced intron element, and a 3′ enhanced exon element. The IRES of the circular and vector constructs comprised of sequences SEQ ID NO: 3283, SEQ ID NO: 3291, SEQ ID NO: 24894, SEQ ID NO: 24895]. The 5′ and 3′ enhanced intron elements comprise a sequence from Anabaena intron. The vector and circular RNA constructs further comprised an V5 antibody tag sequence upstream to the expression sequence but downstream to the TRES.

Circular RNAs were encapsulated into LNPs comprising an ionizable lipid of Lipid 123 of Table B, ethanol, exemplary helper lipid, cholesterol, and PEGylated lipids. The circular RNA encapsulated in LNPs were then intramuscularly injected into female mdx mice (aged 6-8 weeks, weighted at 18 to 22 g). As a control, 3 μL of dystrophin human skeletal muscle cell lysate was spiked into 200 μL PBS tissue. Gastrocnemius muscle, quadricep, diaphragm, splenic, and liver tissue samples were extracted from the mice either 1 day or 6 days after the injection of the circular RNA. Immunoprecipitation and western blot analysis was conducted thereafter.

### Muscle Tissue Lysis Preparation

Quadriceps muscles were collected 48 hr post intramuscular injection and flash frozen in liquid nitrogen. For 1 whole quadriceps muscle, 2 mL of RIPA buffer (Thermo) was supplemented with proteinase inhibitor cocktail (cOmplete Sigma). RIPA was placed in a gentleMACS M-tube (Miltenyi Biotec) on ice and a whole quadriceps muscle was added. Tube was placed in the gentleMACS Octo Dissociator (Miltenyi Biotec) and Protein_01 protocol selected. After dissociation tubes were placed on ice for 5 mins and then centrifuged at 4,000×g for 5 minutes to collect supernatant. Supernatant was then placed in a 1.5 ml tube and spun again at 10,000×g for 10 minutes to collect debris. Supernatant is protein lysate.

### Bradford Assay

To quantify protein concentrations 5 μL of pre-diluted bovine serum albumin (BSA) standards (Thermo) were loaded onto a 96-well plate in triplicate. Protein lysates were loaded at 1 ul in triplicate as well. Room temperature Quick Start Bradford Dye (BioRad) was added at 200 μL per well and allowed to sit for at least 5 mins. Absorbance was read at 595 nm using Cytation 5 plate reader (Bio-tek). Concentrations were calculated based on a standard curve.

### Western Blot Analysis

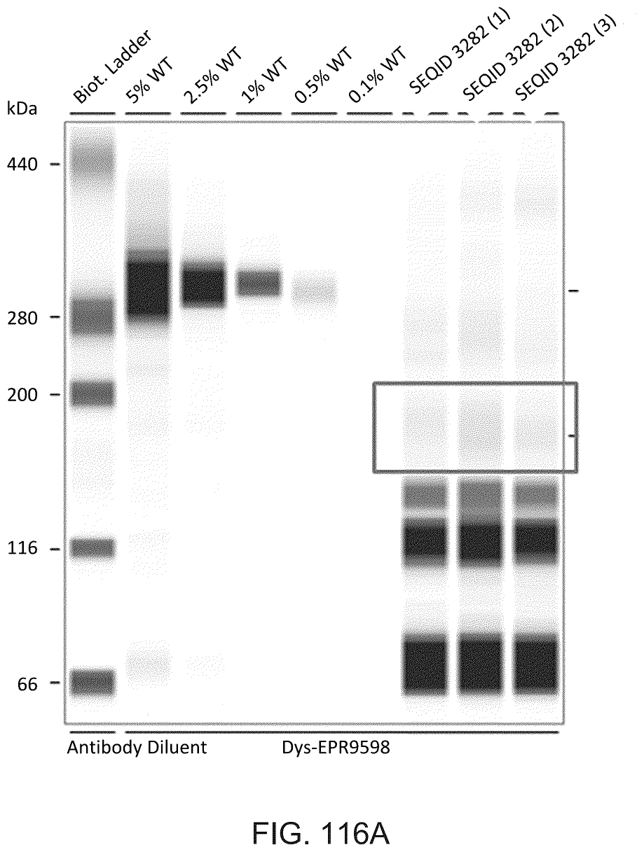

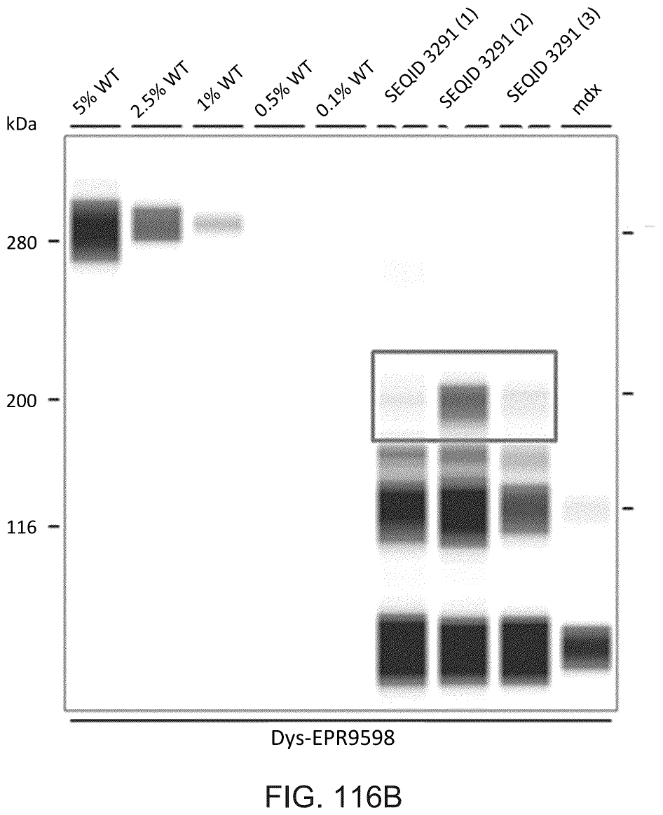

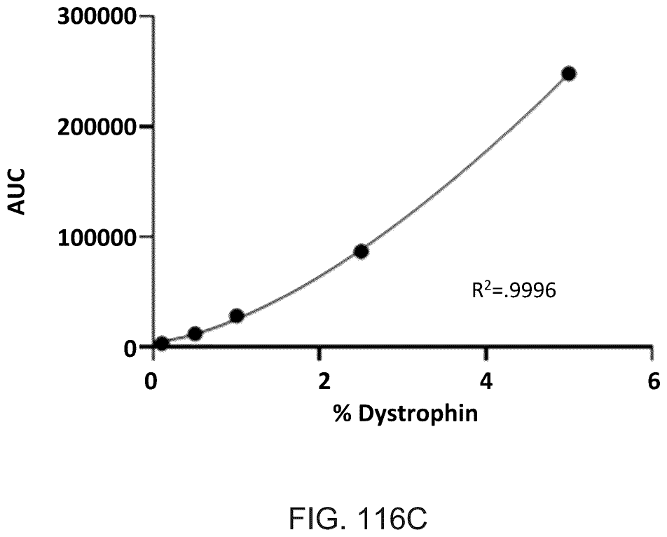

For the western blot analysis, protein lysate samples (cell or tissue) were made up at 1 μg/μL using NuPAGE 4× sample buffer (Thermo) and 1× NuPAGE reducing agent (Thermo). Samples were then heated at 95° C. for 8 minutes and spun down before loading. Samples were then loaded onto a 3-8% Tris-Acetate gel (Thermo), placed in a XCell SureLock Mini-Cell Module (Thermo) and run at 80V for 10 mins, the module was then placed on ice and run at 120V for 80 mins. Proteins were then transferred to a nitrocellulose membrane (Thermo) using a wet transfer system (BioRad) in the cold room for 2 hrs at 80V. Membranes were then taken out and blocked in Intercept blocking buffer (LICOR) for 1 hr and then placed in primary antibody (e.g., anti-V5 tag and/or vinculin recombinant antibody) diluted in Intercept T20 antibody diluent (LICOR) overnight at 4° C. The next day, the membranes were washed 3 times for 5 mins in 1× TBST, secondary antibody was then added at 1:5000 (e.g, anti-dystrophin antibody, goat anti-mouse 800, or goat anti mouse 680) for 1 hr at room temperature. The membranes were then washed 3×5 mins in 1× TBST and imaged using Odyssey CtX. Western blots result for the circular RNAs comprising either SEQ ID NO: 3282 and SEQ ID NO: 3291 are shown in FIGS. 116A-C.

### Immunoprecipitation

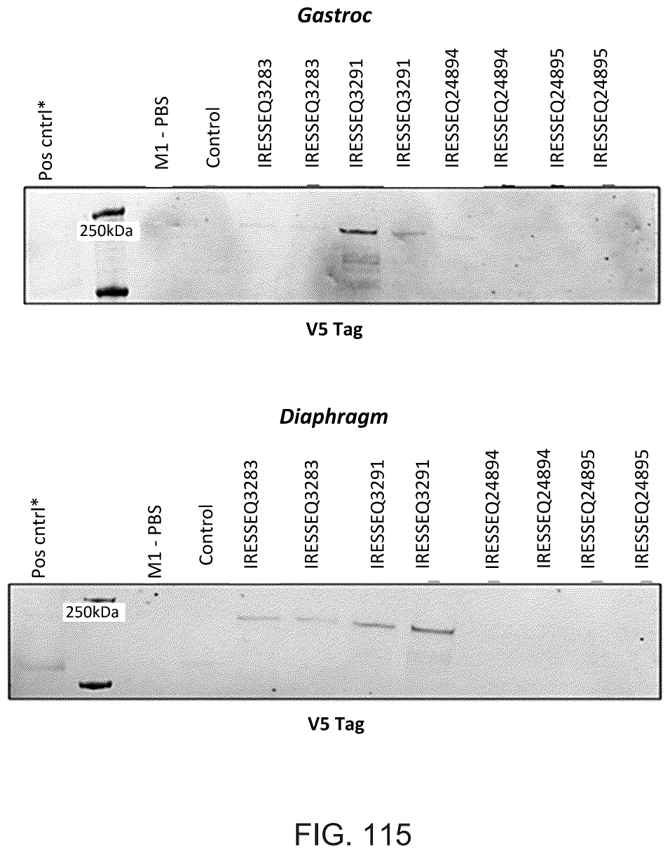

Immunoprecipitation of V5-tag was carried out by first adding 7.5 ug of anti-V5-tag antibody (abcam) to 1-1.5 mg of protein lysate (250 μL total volume) and incubating overnight at 4° C. 100 μL of Pierce protein A/G magnetic agarose beads (Thermo) were magnetized using DynaMag-2 magnet (Invitrogen) and washed twice with 1×PBS-0.1% triton. Lysate-antibody mix was added to the magnetized beads, mixed gently and incubated at 4° C. for 3 hours. Beads were then magnetized and supernatant discarded, beads were washed 5× with 700 μL of 1×PBS-0.1% triton and magnetized again. 20 μL of 4× NuPAGE sample buffer and 1× NuPAGE reducing agent (Thermo) was added to re-suspend the beads, heated to 90° C. for 7 minutes and vortexed vigorously to detach protein from bead. Beads were magnetized and supernatant moved to a clean tube to later run on a western blot as described above. Blot was probed for dystrophin using a rabbit anti-dystrophin antibody (abcam). Immunoprecipitation results for the gastrocnemius muscle and diaphragm are shown in FIGS. 115A-B.

### Jess Assay:

EZ Standard pack reagents were prepared according to manufacturer directions (bio-techne). Protein lysate samples (cell or tissue) were diluted using 4-parts 0.1× sample buffer (bio-techne) combined with 1-part 5× fluorescent master mix. Samples were denatured in a 95° C. heat block, then spun down before loading. Equal parts luminol-S (bio-techne) and peroxide (bio-techne) were combined by pipetting up and down in a microcentrifuge tube and storing on ice. Primary antibodies were diluted in antibody diluent 2 (bio-techne) in a microcentrifuge tube. If two primary antibodies were used, secondary antibodies were combined in equal parts anti-rabbit secondary HRP antibody (bio-techne) and anti-mouse secondary HRP antibody (bio-techne) in a microcentrifuge tube. Reagents were added to the 66-440 kDa Pre-filled Plate (bio-techne) according to the manufacturer's protocol. The 66-440 kDa Pre-filled Plate was spun for 5 minutes at 2500 rpm, then inserted in the Jess machine. Capillary Cartridges (bio-techne) were inserted into the Jess machine. The Jess was run using the 66-440 kDa separation on the chemiluminescence channel.

### Example 92

### Expression of Circular RNAs Comprising an IRES in the Liver or Spleen

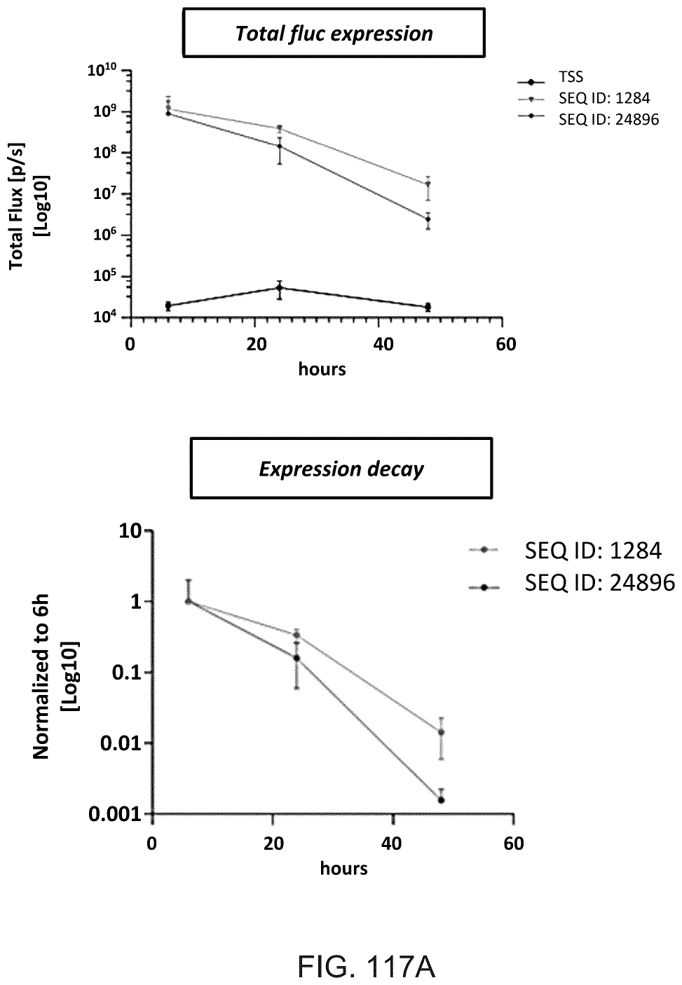

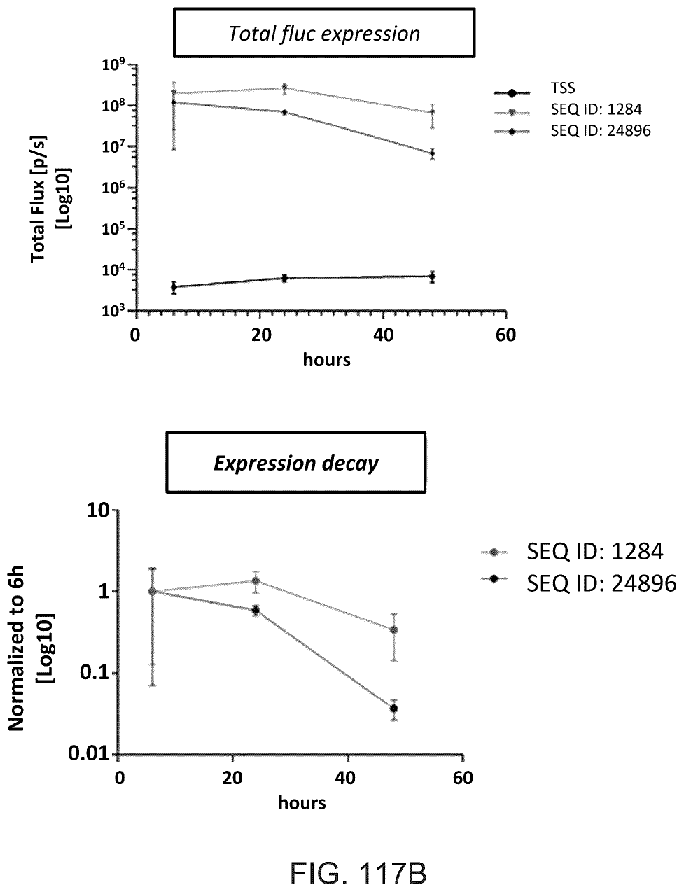

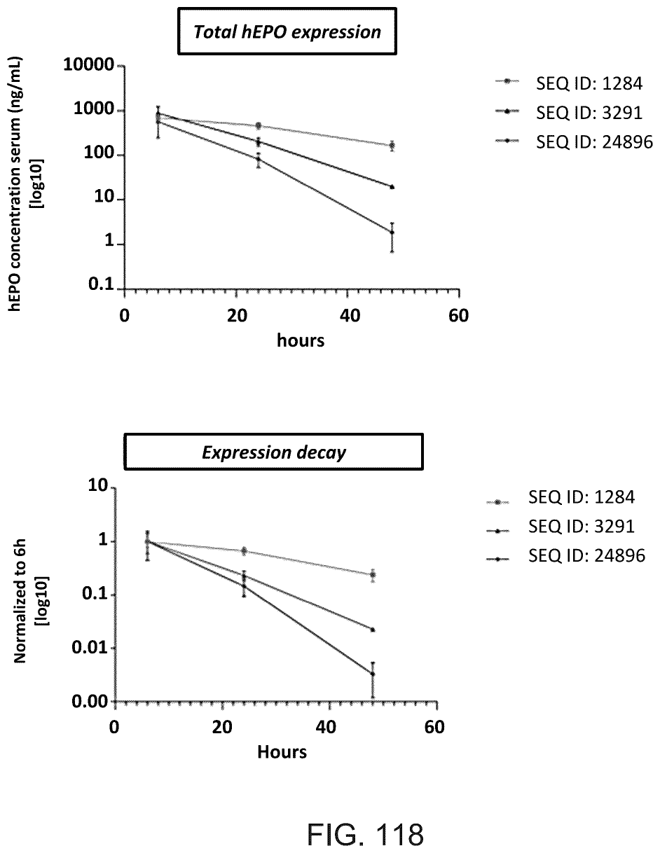

Circular RNA comprised a IRES with a sequence of either SEQ ID NO: 1284, SEQ ID NO: 24896, SEQ ID NO: 3282, SEQ ID NO: 3291 and an expression sequence encoding for firefly luciferase (fluc) or human erythropoietin (hEPO). The circular RNAs were then encapsulated within a LNP comprising ionizable lipid Lipid 123. Wild type Bab1/C mice were dosed with the circular RNAs encapsulated within the LNPs at 0.5 mpk. A terminal bleed was conducted at 6 hours, 24 hours, and 48 hours post administration of the circular RNA-LNP. Post 24-hr transfection, plate was removed from the incubator and allowed to equilibrate to room temperature for 15 mins. A volume of 100 μL of fluc one-step glow assay working solution (Pierce) was added to each well. The plate was placed on a plate shaker and mixed at 250 rpm for 3 min. Post-mixing, the plate was placed at room temperature for 10 min. Luminescence was read using a Cytation 5 instrument (Bio-Tek). Ex-vivo IVIS was conducted on the mice's liver and spleen. hEPO ELISA analysis was conducted to determine serum hEPO concentrations. FIGS. 117A and B provide the liver and splenic total flu expression and expression decay of the circular RNAs. FIG. 118 provides the hEPO expression and expression decay of the circular RNAs.

### Example 93

Expression of Circular RNAs Comprising an IRES In Vitro within Primary Human Hepatocytes

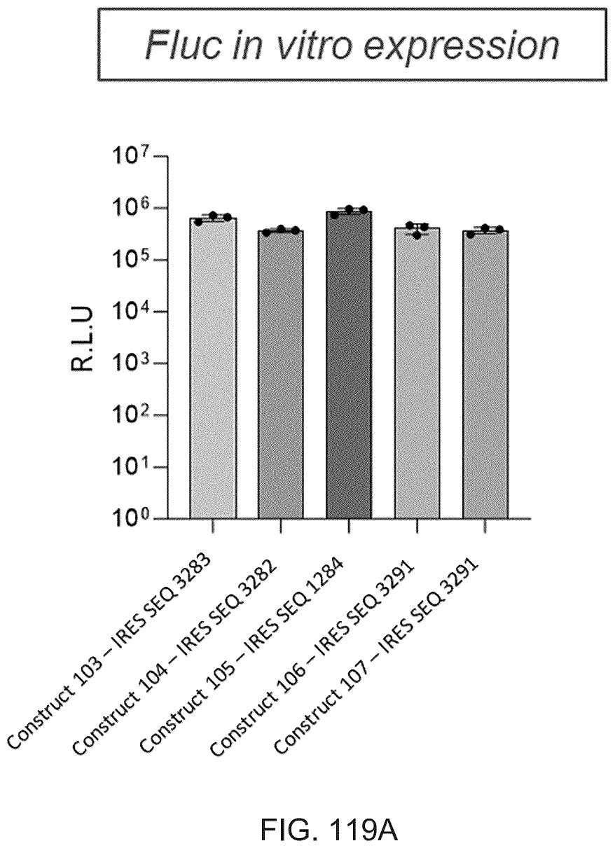

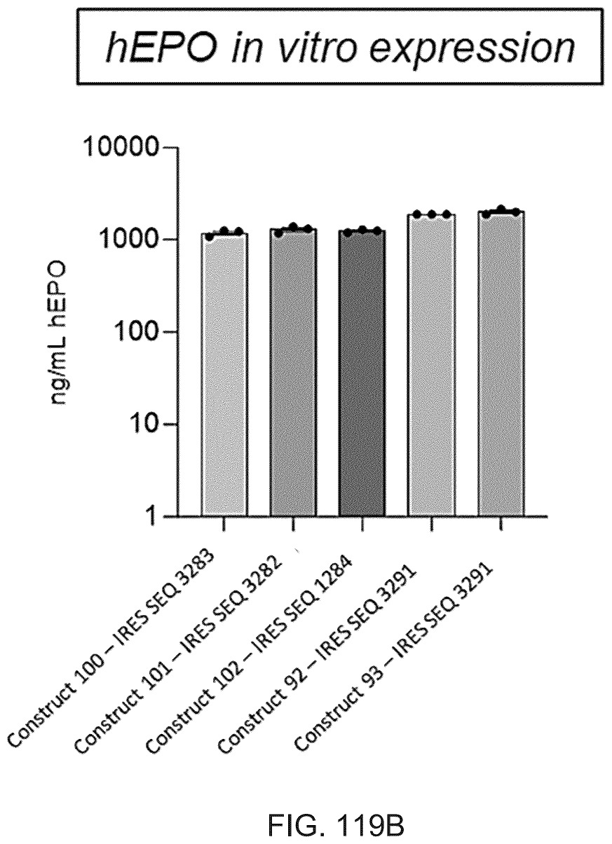

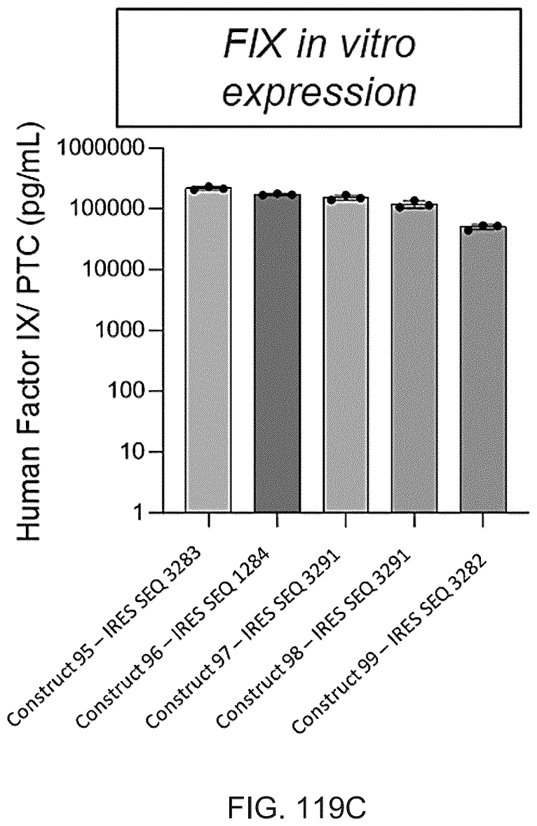

Circular RNA comprised a TRES with a sequence of either SEQ ID NO: 1284, SEQ ID NO: 24896, SEQ ID NO: 3283, SEQ ID NO: 3282, SEQ ID NO: 3291 and an expression sequence encoding for firefly luciferase (fluc), human erythropoietin (hEPO), or human factor X (FIX). Primary human hepatocytes were transfected with 50 ng of the circular RNA using lipofectamine messenger max reagent. After 24 hours following transfection, fluc, hEPO and FIX protein expression was measured using Promega one-glo reagent, hEPO ELISA, and FIX ELISA respectively. FIGS. 119A-C depicts the expression of fluc, hEPO, and FIX in the primary human hepatocytes.

### Incorporation by Reference

All publications, patents, and patent applications mentioned in this specification are herein incorporated by reference to the same extent as if each individual publication, patent, or patent application was specifically and individually indicated as being incorporated by reference herein.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

What is claimed is:

1. A circular RNA polynucleotide (oRNA) comprising a translation initiation element (TIE), wherein the TIE comprises a sequence having at least 85% sequence identity to a sequence set forth in any one of SEQ ID NOS: 14067-24829 (GIRES-1 through GIRES-10762), or a fragment thereof, or having a consensus sequence set forth in any one of SEQ ID NOS: 24867-24892, optionally barcoded with a barcode sequence selected from SEQ ID NOs: 3304-14066; or a precursor RNA polynucleotide capable of producing said oRNA.

2. The oRNA or precursor RNA polynucleotide of claim 1, wherein the TIE comprises an internal ribosome entry site (IRES) or a fragment thereof.

3. The oRNA or precursor RNA polynucleotide of claim 1 or 2, wherein the IRES is in whole or in part from an untranslated region (UTR).

4. The oRNA or precursor RNA polynucleotide of any one of claims 2-3, wherein the IRES has at least 90% identity to a sequence set forth in any one of SEQ ID NOS: 14067-24829.

5. The oRNA or precursor RNA polynucleotide of any one of claims 2-4, wherein the IRES has at least 95% identity to a sequence set forth in any one of SEQ ID NOS: 14067-24829.

6. The oRNA or precursor RNA polynucleotide of any one of claims 2-5, wherein the IRES has at least 98% identity to a sequence set forth in any one of SEQ ID NOS: 14067-24829.

7. The oRNA or precursor RNA polynucleotide of any one of claims 2-6, wherein the IRES comprises a sequence set forth in any one of SEQ ID NOS: 14067-24829.

8. An oRNA or precursor RNA polynucleotide capable of producing the oRNA, comprising a core functional element, and a pharmaceutically acceptable salt, buffer, diluent, or combination thereof, wherein the core functional element comprises a translation initiation element (TIE), wherein the TIE comprises at least 85% sequence identity to a sequence set forth in any one of SEQ ID NOs: 793, 876, 1017, 1216, and 3291, wherein the oRNA is capable of expressing a therapeutic protein in a T cell.

9. An oRNA or precursor RNA polynucleotide capable of producing the oRNA, comprising a core functional element, and a pharmaceutically acceptable salt, buffer, diluent, or combination thereof, wherein the core functional element comprises a translation initiation element (TIE), wherein the TIE comprises at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, wherein the oRNA is capable of expressing a therapeutic protein in a T cell.

10. An oRNA or precursor RNA polynucleotide capable of producing the oRNA, comprising a core functional element, and a pharmaceutically acceptable salt, buffer, diluent, or combination thereof, wherein the core functional element comprises a translation initiation element (TIE), wherein the TIE comprises at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, wherein the oRNA is capable of expressing a therapeutic protein in a T cell.

11. The oRNA or precursor RNA polynucleotide of any one of claims 8-10, wherein the TIE comprises an internal ribosome entry site (IRES) or a fragment thereof.

12. The oRNA or precursor RNA polynucleotide of claim 11, wherein the IRES is in whole or in part from an untranslated region (UTR).

13. The oRNA or precursor RNA polynucleotide of claim 11 or 12, wherein the IRES sequence has at least 90% sequence identity to a sequence set forth in any one of SEQ ID NO: 793, 876, 1017, 1216, and 3291.

14. The oRNA or precursor RNA polynucleotide of any one of claims 11-13, wherein the IRES sequence has at least 95% sequence identity to a sequence set forth in any one of SEQ ID NO: 793, 876, 1017, 1216, and 3291.

15. The oRNA or precursor RNA polynucleotide of any one of claims 11-14, wherein the IRES sequence has at least 98% sequence identity to a sequence set forth in any one of SEQ ID NO: 793, 876, 1017, 1216, and 3291.

16. The oRNA or precursor RNA polynucleotide of any one of claims 11-15, wherein the IRES sequence has at least 99% sequence identity to a sequence set forth in any one of SEQ ID NO: 793, 876, 1017, 1216, and 3291.

17. The oRNA or precursor RNA polynucleotide of any one of claims 11-16, wherein the IRES sequence comprises a sequence set forth in any one of SEQ ID NO: 793, 876, 1017, 1216, and 3291.

18. The oRNA or precursor RNA polynucleotide of claim 11 or 12, wherein the IRES sequence has at least 90% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

19. The oRNA or precursor RNA polynucleotide of any one of claim 11, 12, or 18, wherein the IRES sequence has at least 95% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

20. The oRNA or precursor RNA polynucleotide of any one of claim 11, 12, 18, or 19 wherein the IRES sequence has at least 98% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

21. The oRNA or precursor RNA polynucleotide of any one of claims 11, 12, or 18-20, wherein the IRES sequence has at least 99% sequence identity to a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

22. The oRNA or precursor RNA polynucleotide of any one of claims 11, 12, or 18-21, wherein the IRES sequence comprises a sequence set forth in any one of SEQ ID NO: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

23. The oRNA or precursor RNA polynucleotide of claim 11 or 12, wherein the IRES sequence has at least 90% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

24. The oRNA or precursor RNA polynucleotide of any one of claims 11, 12, or 23, wherein the IRES sequence has at least 95% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

25. The oRNA or precursor RNA polynucleotide of any one of claim 11, 12, 23, or 24, wherein the IRES sequence has at least 98% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

26. The oRNA or precursor RNA polynucleotide of any one of claims 11, 12, 23-25, wherein the IRES sequence has at least 99% sequence identity to a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

27. The oRNA or precursor RNA polynucleotide of any one of claims 11, 12, 23-26, wherein the IRES sequence comprises a sequence set forth in any one of SEQ ID NO: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

28. The oRNA or precursor RNA polynucleotide of any one of the preceding claims, wherein the precursor RNA further comprises an accessory element.

29. The oRNA or precursor RNA polynucleotide of claim 28, wherein the accessory element comprises a miRNA binding site or a fragment thereof, a restriction site or a fragment thereof, an RNA editing motif or a fragment thereof, a zip code element or a fragment thereof, an RNA trafficking element or a fragment thereof, an endonuclease site or a fragment thereof, or a combination thereof.

30. The oRNA or precursor RNA polynucleotide of claim 28 or 29, wherein the accessory element comprises a binding domain to an IRES transacting factor (ITAF) and/or a translation initiation factor.

31. The oRNA or precursor RNA polynucleotide of claim 30, wherein the binding domain comprises a polyA region, a polyC region, a polyAC region, a polypyrimidine tract, or a combination or variant thereof.

32. The oRNA or precursor RNA polynucleotide of claim 30 or 31, wherein the ITAF comprises a poly(rC)-binding protein 1 (PCBP1), PCBP2, PCBP3, PCBP4, poly(A)-binding protein 1 (PABP1), polypyrimidine-tract binding protein (PTB), Argonaute protein family member, HNRNPK (heterogeneous nuclear ribonucleoprotein K protein), or La protein, or a fragment or combination thereof.

33. The oRNA or precursor RNA polynucleotide of any one of the preceding claims, wherein the core functional element further comprises a coding sequence and, optionally, a termination sequence located downstream to the coding sequence.

34. The oRNA or precursor RNA polynucleotide of claim 33, wherein the coding sequence is located downstream to the IRES.

35. The oRNA or precursor RNA polynucleotide of claim 33, wherein the coding sequence is located upstream to the IRES.

36. The oRNA or precursor RNA polynucleotide of any one of claims 33-35, wherein the termination sequence is a stop codon or a stop cassette.

37. The oRNA or precursor RNA polynucleotide of claim 36, wherein the stop cassette comprises one or more stop codons in two or more open reading frames.

38. The precursor RNA polynucleotide of any one of the preceding claims, wherein the precursor RNA polynucleotide comprises:
a. a 5′ enhanced intron element,
b. a 5′ enhanced exon element,
c. the core functional element,
d. a 3′ enhanced exon element, and
e. a 3′ enhanced intron element.

39. The precursor RNA polynucleotide of claim 38, wherein elements (a)-(e) are arranged in order from (a) to (e).

40. The precursor RNA polynucleotide of claim 38 or 39, wherein the 5′ enhanced exon element and/or the 3′ enhanced exon element are each comprised within the core functional element.

41. The precursor RNA polynucleotide of claim 40, wherein the 5′ enhanced exon element and/or the 3′ enhanced exon element are each comprised within the coding sequence.

42. The precursor RNA polynucleotide of any one of claims 38-41, wherein the 5′ enhanced intron element comprises a 3′ intron segment.

43. The precursor RNA polynucleotide of claim 42, wherein the 3′ intron segment further comprises a first or a first and a second nucleotides of a 3′ group I intron splice site dinucleotide.

44. The precursor RNA polynucleotide of claim 42 or 43, wherein the 3′ intron segment is located at the 3′ end of the 5′ enhanced intron element.

45. The precursor RNA polynucleotide of any one of claims 42-44, wherein the 5′ enhanced intron element comprises a leading untranslated sequence located at the 5′ end.

46. The precursor RNA polynucleotide of claim 45, wherein the leading untranslated sequence comprises a spacer.

47. The precursor RNA polynucleotide of claim 45 or 46, wherein the leading untranslated sequence comprises the last nucleotide of a transcription start site.

48. The precursor RNA polynucleotide of any one of claims 45-47, wherein the leading untranslated sequence comprises 1 to 100 additional nucleotides.

49. The precursor RNA polynucleotide of any one of claims 42-48, wherein the 5′ enhanced intron element comprises a 5′ affinity sequence.

50. The precursor RNA polynucleotide of claim 49, wherein the 5′ affinity sequence comprises a polyA, polyAC, or polypyrimidine sequence.

51. The precursor RNA polynucleotide of claim 49 or 50, wherein the 5′ affinity sequence comprises 10 to 100 nucleotides.

52. The precursor RNA polynucleotide of any one of claims 38-51, wherein the 5′ enhanced intron element comprises a 5′ external spacer sequence.

53. The precursor RNA polynucleotide of claim 52, wherein the 5′ external spacer sequence is located between the 5′ affinity sequence and the 3′ intron segment.

54. The precursor RNA polynucleotide of claim 52 or 53, wherein the 5′ external spacer sequence has a length of about 6 to 60 nucleotides.

55. The precursor RNA polynucleotide of any one of claims 52-54, wherein the 5′ external spacer sequence comprises or consists of a sequence selected from SEQ ID NOs: 3094-3152.

56. The precursor RNA polynucleotide of any one of claims 38-55, wherein the 5′ enhanced intron element comprises:
a. a leading untranslated sequence;
b. a 5′ affinity sequence;
c. a 5′ external spacer sequence; and
d. a 3′ intron segment including the first nucleotide of a 3′ Group I intron splice site;
wherein the leading untranslated sequence comprises the last nucleotide of a transcription start site and 1 to 100 nucleotides.

57. The precursor RNA polynucleotide of claim 56, wherein (a)-(d) are arranged in the order from (a) to (d).

58. The precursor RNA polynucleotide of any one of claims 38-55, wherein the 5′ enhanced intron element comprises:
a. a leading untranslated sequence;
b. a 5′ external spacer sequence;
c. a 5′ affinity sequence; and
d. a 3′ intron segment including the first nucleotide of a 3′ group I splice site;
wherein the leading untranslated sequence comprises the last nucleotide of a transcription start site and 1 to 100 nucleotide.

59. The precursor RNA polynucleotide of claim 58, wherein (a)-(d) are arranged in the order from (a) to (d).

60. The precursor RNA polynucleotide of any one of claims 38-55, wherein the 5′ enhanced intron element comprises:
a. a leading untranslated sequence;
b. a 5′ external spacer sequence;
c. a 5′ affinity sequence; and
d. a 3′ intron segment including the first and second nucleotides of a 3′ Group I splice site;
wherein the leading untranslated sequence comprises the last nucleotide of a transcription start site and 1 to 100 nucleotides; and wherein the 5′ enhanced exon element comprises a 3′ exon segment lacking the second nucleotide of a 3′ group I splice site dinucleotide.

61. The precursor RNA polynucleotide of claim 60, wherein (a)-(d) are arranged in the order from (a) to (d).

62. The precursor RNA polynucleotide of any one of claims 38-61, wherein the 5′ enhanced exon element comprises a 3′ exon segment.

63. The precursor RNA polynucleotide of claim 62, wherein the 3′ exon segment further comprises the second nucleotide of a 3′ group I intron splice site dinucleotide.

64. The precursor RNA polynucleotide of claim 62 or 63, wherein the 3′ exon segment comprises 1 to 100 natural nucleotides derived from a natural exon.

65. The precursor RNA polynucleotide of claim 64, wherein the natural exon is derived from a Group I intron containing gene or a fragment thereof.

66. The precursor RNA polynucleotide of claim 64 or 65, wherein the natural exon derived from an anabaena bacterium, T4 phage virus, twort bacteriophage, tetrahymena, or azoarcus bacterium.

67. The precursor RNA polynucleotide of any of claims 38-66, wherein the 5′ enhanced exon element comprises a 5′ internal spacer sequence located downstream from the 3′ exon segment.

68. The precursor RNA polynucleotide of claim 67, wherein the 5′ internal spacer sequence is about 6 to 60 nucleotides in length.

69. The precursor RNA polynucleotide of claim 67 or 68, wherein the 5′ internal spacer sequence comprises or consists of a sequence selected from SEQ ID NOs: 3094-3152.

70. The precursor RNA polynucleotide of any one of claims 38-69, wherein the 5′ enhanced exon element comprises in the following order:
a. a 3′ exon segment including the second nucleotide of a 3′ group I intron splice site dinucleotide; and
b. a 5′ internal spacer sequence,
wherein the 3′ exon segment comprises 1 to 100 natural nucleotides derived from a natural exon.

71. The precursor RNA polynucleotide of any one of claims 38-69, wherein the 5′ enhanced exon element comprises in the following order:
a. a 3′ exon segment; and
b. a 5′ internal spacer sequence,
wherein the 3′ exon segment comprises 1 to 100 natural nucleotides derived from a natural exon; and wherein the 5′ enhanced intron element comprises a 3′ intron segment comprising the first and second nucleotides of a 3′ group I splice site dinucleotide.

72. The precursor RNA polynucleotide of any one of claims 38-71, wherein the 3′ enhanced exon element comprises a 5′ exon segment.

73. The precursor RNA polynucleotide of claim 72, wherein the 5′ exon segment comprises the first nucleotide of a 5′ group I intron segment.

74. The precursor RNA polynucleotide of claim 72 or 73, wherein the 5′ exon segment further comprises 1 to 100 nucleotides derived from a natural exon.

75. The precursor RNA polynucleotide of claim 74, wherein the natural exon is derived from a Group I intron containing gene or a fragment thereof.

76. The precursor RNA polynucleotide of any one of claims 38-75, wherein the 3′ enhanced exon element comprises a 3′ internal spacer sequence.

77. The precursor RNA polynucleotide of claim 76, wherein the 3′ internal spacer sequence is located between the termination sequence and the 5′ exon segment.

78. The precursor RNA polynucleotide of claim 76 or 77, wherein the 3′ internal spacer is about 6 to 60 nucleotides in length.

79. The precursor RNA polynucleotide of any one of claims 76-78, wherein the 3′ internal spacer comprises or consists of a sequence selected from SEQ ID NOs: 3094-3152.

80. The precursor RNA polynucleotide of any one of claims 38-79, wherein the 3′ enhanced exon element comprises:
a. a 3′ internal spacer sequence; and
b. a 5′ exon segment including the first nucleotide of a 5′ group I intron splice site dinucleotide,
wherein the 5′ exon segment comprises 1 to 100 nucleotides derived from a natural exon.

81. The precursor RNA polynucleotide of any one of claims 38-79, wherein the 3′ enhanced exon element comprises:
a. a 3′ internal spacer sequence; and
b. a 5′ exon segment,
wherein the 5′ exon segment comprises 1 to 100 nucleotides derived from a natural exon; wherein the 3′ enhanced intron element comprises a 5′ intron segment comprising the first and second nucleotide of a 5′ group I intron splice site dinucleotide.

82. The precursor RNA polynucleotide of any one of claims 38-81, wherein the 3′ enhanced intron element comprises a 5′ intron segment.

83. The precursor RNA polynucleotide of claim 82, wherein the 5′ intron segment comprises a second nucleotide of a 5′ group I intron splice site dinucleotide.

84. The precursor RNA polynucleotide of any one of claims 38-83, wherein the 3′ enhanced intron element comprises a trailing untranslated sequence located at the 3′ end of the 5′ intron.

85. The precursor RNA polynucleotide of claim 84, wherein the trailing untranslated sequence comprises 3 to 12 nucleotides.

86. The precursor RNA polynucleotide of any of claims 38-85, wherein the 3′ enhanced intron element comprises a 3′ external spacer sequence.

87. The precursor RNA polynucleotide of claim 86, wherein the 3′ external spacer sequence is located between the 5′ intron segment and trailing untranslated sequence.

88. The precursor RNA polynucleotide of claim 86 or 87, wherein the 3′ external spacer sequence has a length of 6 to 60 nucleotides in length.

89. The precursor RNA polynucleotide of any of claims 86-88, wherein the 3′ external spacer sequence comprises or consists of a sequence selected from SEQ ID NOs: 3094-3152.

90. The precursor RNA polynucleotide of any of claims 38-89, wherein the 3′ enhanced intron element comprises a 3′ affinity sequence.

91. The precursor RNA polynucleotide of claim 90, wherein the 3′ affinity sequence is located between the 3′ external spacer sequence and the trailing untranslated sequence.

92. The precursor RNA polynucleotide of claim 90 or 91, wherein the 3′ affinity sequence comprises a polyA, polyAC, or polypyrimidine sequence.

93. The precursor RNA polynucleotide of any one of claims 90-92, wherein the affinity sequence comprises 10 to 100 nucleotides.

94. The precursor RNA polynucleotide of any one of claims 38-93, wherein the 5′ enhanced intron element further comprises a 5′ external duplex sequence; wherein the 3′ enhanced intron element further comprises a 3′ external duplex sequence.

95. The precursor RNA polynucleotide of claim 94, wherein the 5′ external duplex sequence and 3′ external duplex sequence are fully or partially complementary to each other.

96. The precursor RNA polynucleotide of claim 94 or 95, wherein the 5′ external duplex sequence comprises fully synthetic or partially synthetic nucleotides.

97. The precursor RNA polynucleotide of any one of claims 94-96, wherein the 3′ external duplex sequence comprises fully synthetic or partially synthetic nucleotides.

98. The precursor RNA polynucleotide of any one of claims 94-97, wherein the 3′ external duplex sequence is about 6 to about 50 nucleotides.

99. The precursor RNA polynucleotide of any one of claims 94-98, wherein the 5′ external duplex sequence is about 6 to about 50 nucleotides.

100. The precursor RNA polynucleotide of any one of claims 38-99, wherein the 5′ enhanced exon element further comprises a 5′ internal duplex sequence; wherein the 3′ enhanced exon element further comprises a 3′ internal duplex sequence.

101. The precursor RNA polynucleotide of claim 100, wherein the 5′ internal duplex sequence and 3′ internal duplex sequence are fully complementary to each other.

102. The precursor RNA polynucleotide of claim 100, wherein the 5′ internal duplex sequence and 3′ internal duplex sequence are partially complementary to each other.

103. The precursor RNA polynucleotide of claim 102, wherein the 5′ internal duplex sequence and 3′ internal duplex sequences form a double-stranded duplex structure comprising at least one mismatched nucleotide pair.

104. The precursor RNA polynucleotide of claim 103, wherein the double-stranded duplex structure comprises at least two mismatched nucleotide pairs.

105. The precursor RNA polynucleotide of claim 103 or 104, wherein the double-stranded duplex structure comprises at least three mismatched nucleotide pairs.

106. The precursor RNA polynucleotide of any one of claims 103-105, wherein the double-stranded duplex structure comprises at least four mismatched nucleotide pairs.

107. The precursor RNA polynucleotide of any one of claims 103-106, wherein the double-stranded duplex structure comprises at least five mismatched nucleotide pairs.

108. The precursor RNA polynucleotide of any one of claims 100-107, wherein the 5′ internal duplex sequence comprises fully synthetic nucleotides.

109. The precursor RNA polynucleotide of any one of claims 100-108, wherein the 5′ internal duplex sequence comprises partially synthetic nucleotides.

110. The precursor RNA polynucleotide of any one of claims 100-109, wherein the 3′ internal duplex sequence comprises fully synthetic nucleotides.

111. The precursor RNA polynucleotide of any one of claims 100-109, wherein the 3′ internal duplex sequence comprises partially synthetic nucleotides.

112. The precursor RNA polynucleotide of any one of claims 100-111, wherein the 3′ internal duplex sequence is about 6 to about 19 nucleotides.

113. The precursor RNA polynucleotide of any one of claims 100-112, wherein the 5′ internal duplex sequence is about 6 to about 19 nucleotides.

114. The precursor RNA polynucleotide of any one of claims 38-113, wherein the 3′ enhanced intron element comprises in the following order:
a. a 5′ intron segment including the second nucleotide of a 5′ group I intron splice site dinucleotide;
b. a 3′ external spacer sequence; and
c. a 3′ affinity sequence.

115. The precursor RNA polynucleotide of anyone of claims 38-114, wherein the 3′ enhanced exon element comprises in the following order:
a. a 5′ intron segment including the first and second nucleotide of a 5′ group I intron splice site dinucleotide;
b. a 3′ external spacer sequence; and
c. a 3′ affinity sequence wherein the 3′ enhanced exon element comprises a 5′ exon segment lacking the first nucleotide of a 5′ group I intron splice site dinucleotide.

116. The precursor RNA polynucleotide of claim 38-115, wherein the precursor RNA polynucleotide comprises:
a. a leading untranslated sequence;
b. a 5′ affinity sequence;
c. 5′ external duplex sequence;
d. 5′ spacer sequence;
e. 3′ intron segment;
f. 3′ exon segment;
g. 5′ internal duplex sequence
h. 5′ internal spacer sequence;
i. a translation initiation element;
j. a coding sequence;
k. a termination sequence;
l. a 3′ internal spacer sequence;
m. a 3′ internal duplex sequence;
n. a 5′ exon segment;
o. a 5′ intron segment;
p. a 3′ external duplex sequence;
q. a 3′ affinity sequence; and
r. a trailing untranslated sequence.

117. The precursor RNA polynucleotide of claim 116, wherein (a)-(r) are arranged in the order from (a) to (r).

118. The precursor RNA polynucleotide of any one of claims 38-115, wherein the precursor RNA polynucleotide comprises:
a. a leading untranslated sequence;
b. a 5′ affinity sequence;
c. a 5′ external spacer sequence;
d. a 3′ intron segment;
e. a 3′ exon segment;
f. a 5′ internal duplex sequence;
g. a 5′ internal spacer sequence;
h. a translation initiation element;
i. a coding sequence;
j. a termination sequence;
k. a 3′ internal spacer sequence;
l. a 3′ internal duplex sequence;
m. a 5′ exon segment;
n. a 5′ intron segment;
o. a 3′ external spacer sequence;
p. a 3′ affinity sequence; and
q. a trailing untranslated sequence.

119. The precursor RNA polynucleotide of claim 118, wherein (a)-(q) are arranged in the order from (a) to (q).

120. The precursor RNA polynucleotide of any one of claims 38-115, wherein the precursor RNA polynucleotide comprises:
a. a leading untranslated sequence;
b. a 5′ affinity sequence;
c. a 5′ external spacer sequence;
d. a 3′ intron segment;
e. a 3′ exon segment;
f. a 5′ internal spacer sequence;
g. a translation initiation element;
h. a coding sequence;
i. a termination sequence;
j. a 3′ internal spacer sequence;
k. a 5′ exon segment;
l. a 5′ intron segment;
m. a 3′ external spacer sequence;
n. a 3′ affinity sequence; and
o. a trailing untranslated sequence.

121. The precursor RNA polynucleotide of claim 120, wherein (a)-(o) are arranged in the order from (a) to (o).

122. The precursor RNA polynucleotide of any one of claims 38-115, wherein the precursor RNA polynucleotide comprises:
a. a leading untranslated sequence;
b. a 5′ affinity sequence;
c. 5′ external duplex sequence;
d. 5′ spacer sequence;
e. 3′ intron segment;
f. 3′ exon segment;
g. 5′ internal duplex sequence
h. 5′ internal spacer sequence;
i. a termination sequence;
j. a coding sequence;
k. a translation initiation element;
l. a 3′ internal spacer sequence;
m. a 3′ internal duplex sequence;
n. a 5′ exon segment;
o. a 5′ intron segment;
p. a 3′ external duplex sequence;
q. a 3′ affinity sequence; and
r. a trailing untranslated sequence.

123. The precursor RNA polynucleotide of claim 122, wherein (a)-(r) are arranged in the order from (a) to (r).

124. The precursor RNA polynucleotide of any one of claims 33-123, wherein the coding sequence comprises two or more protein coding regions.

125. The precursor RNA polynucleotide of claim 124, wherein the coding sequence comprises a sequence encoding a proteolytic cleavage site and/or a ribosomal stuttering element between the first and second expression sequence.

126. The precursor RNA polynucleotide of claim 125, wherein the ribosomal stuttering element is a self-cleaving spacer.

127. The precursor RNA polynucleotide of claim 125 or 126, comprising a polynucleotide sequence encoding 2A ribosomal stuttering peptide.

128. The precursor RNA polynucleotide of any one of the preceding claims, wherein the precursor RNA polynucleotide comprises the following sequences operably linked to the IRES and/or operable linked to one another: (1) a 3′ group I intron segment; (2) a coding sequence that encodes the therapeutic protein; and (3) a 5′ group I intron segment.

129. The precursor RNA polynucleotide of claim 128, wherein the 3′ group I intron segment and the 5′ group I intron segment are each derived from a bacterial phage, a viral vector, an organelle genome, or a nuclear rDNA gene.

130. The precursor RNA polynucleotide of claim 129, wherein the 3′ group I intron segment and the 5′ group I intron segment are each derived from an anabaena bacterium, a T4 phage virus, a twort bacteriophage, a tetrahymena, or an azoarcus bacterium.

131. The precursor RNA polynucleotide of any one of the preceding claims, wherein the precursor RNA polynucleotide comprises one or more spacer sequences, said one or more spacer sequences being operably connected to at least one of the 3′ group I intron segment, IRES sequence, coding sequence, and 5′ group I intron segment.

132. The precursor RNA polynucleotide of claim 131, wherein the precursor RNA polynucleotide comprises two spacer sequences.

133. The precursor RNA polynucleotide of claim 132, wherein the two spacer sequences comprise a 5′ external spacer sequence and a 3′ external spacer sequence, or a 5′ internal spacer sequence and a 3′ internal spacer sequence.

134. The precursor RNA polynucleotide of claim 131, wherein the precursor RNA polynucleotide comprises four spacer sequences.

135. The precursor RNA polynucleotide of claim 134, wherein the four spacer sequences comprise a 5′ external spacer sequence, a 3′ external spacer sequence, a 5′ internal spacer sequence, and a 3′ internal spacer sequence.

136. The precursor RNA polynucleotide of any one of the preceding claims, wherein the precursor RNA polynucleotide comprises a 5′ internal duplex sequence and a 3′ internal duplex sequence.

137. The precursor RNA polynucleotide of claim 136, wherein the 5′ internal duplex sequence and 3′ internal duplex sequence are fully or partially complementary to each other.

138. The precursor RNA polynucleotide of any one of the preceding claims, wherein the precursor RNA polynucleotide comprises a polyA region, polyC region, polyAC region, polypyrimidine tract, or a combination or variant thereof.

139. The precursor RNA polynucleotide of any one of the preceding claims, wherein the precursor RNA polynucleotide comprises a 3′ exon segment and a 5′ exon segment, each derived from a natural exon.

140. The precursor RNA polynucleotide of claim 136, wherein the precursor RNA polynucleotide comprises the following elements operably linked to one another:
(a) the 5′ external spacer sequence;
(b) the 3′ group I intron segment;
(c) the 5′ exon segment;
(d) the 5′ internal duplex sequence;
(e) the IRES sequence;
(f) the coding sequence;
(g) the 3′ internal duplex sequence;
(h) the 3′ exon segment;
(j) the 5′ group I intron segment; and
(k) the 3′ external spacer sequence.

141. The precursor RNA polynucleotide of claim 140, wherein elements (a)-(k) are arranged in the order of (a)-(k).

142. The precursor RNA polynucleotide of claim 136, wherein the precursor RNA polynucleotide comprises the following elements operably linked to one another:
(a) the 3′ group I intron segment;
(b) the 5′ exon segment;
(c) the 5′ internal duplex sequence;
(d) the 5′ internal spacer sequence;
(e) the IRES sequence;
(f) the coding sequence;
(g) the 3′ internal spacer sequence
(h) the 3′ internal duplex sequence;
(i) the 3′ exon segment; and
(j) the 5′ group I intron segment.

143. The precursor RNA polynucleotide of claim 142, wherein elements (a)-(j) are arranged in the order of (a)-(j).

144. The precursor RNA polynucleotide of claim 136, wherein the precursor RNA polynucleotide comprises the following elements operably linked to one another:
(a) the 5′ external spacer sequence;
(b) the 3′ group I intron segment;
(c) the 5′ exon segment;
(d) the 5′ internal duplex sequence;
(e) the 5′ internal spacer sequence;
(f) the IRES sequence;
(g) the coding sequence;
(h) the 3′ internal spacer sequence
(i) the 3′ internal duplex sequence;
(j) the 5′ exon element;
(k) the 5′ group I intron segment; and
(l) the 3′ external spacer sequence.

145. The precursor RNA polynucleotide of claim 144, wherein elements (a)-(l) are arranged in the order of (a)-(l).

146. The oRNA or precursor RNA polynucleotide of any one of the preceding claims, wherein the precursor RNA polynucleotide comprises partially synthetic nucleotides.

147. The oRNA or precursor RNA polynucleotide of any one of claims 1-145, wherein the precursor RNA polynucleotide comprises fully synthetic nucleotides.

148. The oRNA or precursor RNA polynucleotide of any one of the preceding claims, wherein the precursor RNA polynucleotide is transcribed from a vector or DNA polynucleotide comprising a PCR product, a linearized plasmid, a non-linearized plasmid, a linearized minicircle, a non-linearized minicircle, a viral vector, a cosmid, a cDNA, or an artificial chromosome.

149. An oRNA produced using the precursor RNA polynucleotide of any one of the preceding claims.

150. The oRNA of claim 149, comprising the IRES sequence and the coding sequence.

151. The oRNA of claim 150, wherein the IRES sequence is upstream of the coding sequence.

152. The oRNA of claim 150, wherein the IRES sequence is downstream of the coding sequence.

153. The oRNA of any one of claims 149-152, comprising:
(a) the 5′ exon segment;
(b) the 5′ internal duplex sequence;
(c) the 5′ internal spacer sequence;
(d) the IRES sequence;
(e) the coding sequence;
(f) the 3′ internal spacer sequence
(g) the 3′ internal duplex sequence; and
(h) the 5′ exon element.

154. The oRNA of claim 153, wherein (a)-(h) are arranged in the order from (a) to (h).

155. A pharmaceutical composition comprising an oRNA comprising an IRES sequence having at least 85% sequence identity to a sequence set forth in any one of SEQ ID NOS: 14067-24829 or having a consensus sequence set forth in any one of SEQ ID NOs: 24867-24892, and a pharmaceutically acceptable salt, buffer, diluent, or combination thereof, wherein the oRNA is capable of expressing a therapeutic protein in a cell or a pharmaceutical composition comprising the oRNA of any one of claims 1-37 or 146-154.

156. A pharmaceutical composition comprising an oRNA comprising an IRES sequence having at least 85% sequence identity to a sequence set forth in any one of SEQ ID NOS: 14067-24829 or having a consensus sequence set forth in any one of SEQ ID NOs: 24867-24892, a cell, and a pharmaceutically acceptable salt, buffer, diluent, or combination thereof, wherein the oRNA is capable of expressing a therapeutic protein in the cell.

157. A pharmaceutical composition comprising an oRNA comprising an IRES sequence having at least 85% sequence identity to a sequence set forth in any one of SEQ ID NOS: 14067-24829 or having a consensus sequence set forth in any one of SEQ ID NOs: 24867-24892, a transfer vehicle capable of delivering the oRNA to a cell, and a pharmaceutically acceptable salt, buffer, diluent, or combination thereof; wherein the oRNA is capable of expressing a therapeutic protein in a cell.

158. The pharmaceutical composition of any one of claims 155-157, wherein the IRES has at least 90% identity to a sequence set forth in any one of SEQ ID NOS: 14067-24829.

159. The pharmaceutical composition of any one of claims 155-158, wherein the IRES has at least 95% identity to a sequence set forth in any one of SEQ ID NOS: 14067-24829.

160. The pharmaceutical composition of any one of claims 155-159, wherein the IRES has at least 98% identity to a sequence set forth in any one of SEQ ID NOS: 14067-24829.

161. The pharmaceutical composition of claim any one of claims 155-160, wherein the IRES has at least 99% identity to a sequence set forth in any one of SEQ ID NOS: 14067-24829.

162. The pharmaceutical composition of claim any one of claims 155-161, wherein the IRES comprises a sequence set forth in any one of SEQ ID NOS: 14067-24829.

163. A pharmaceutical composition comprising an oRNA comprising an IRES sequence having at least 85% sequence identity to a sequence set forth in any one of SEQ ID NO: 793, 876, 1017, 1216, and 3291, and a pharmaceutically acceptable salt, buffer, diluent, or combination thereof, wherein the oRNA is capable of expressing a therapeutic protein in a T cell.

164. A pharmaceutical composition comprising an oRNA comprising an IRES sequence having at least 85% sequence identity to a sequence set forth in any one of SEQ ID NO: 793, 876, 1017, 1216, and 3291, a T cell, and a pharmaceutically acceptable salt, buffer, diluent, or combination thereof.

165. A pharmaceutical composition comprising an oRNA comprising an IRES sequence having at least 85% sequence identity to a sequence set forth in any one of SEQ ID NO: 793, 876, 1017, 1216, and 3291, a transfer vehicle capable of delivering the oRNA into a T cell, and a pharmaceutically acceptable salt, buffer, diluent, or combination thereof.

166. The pharmaceutical composition of any one of claims 163-165 or 155, wherein the IRES sequence has at least 90% sequence identity to a sequence set forth in any one of SEQ ID NOs: 793, 876, 1017, 1216, and 3291.

167. The pharmaceutical composition of any one of claims 163-166 or 155, wherein the IRES sequence has at least 95% sequence identity to a sequence set forth in any one of SEQ ID NOs: 793, 876, 1017, 1216, and 3291.

168. The pharmaceutical composition of any one of claims 163-167 or 155, wherein the IRES sequence has at least 98% sequence identity to a sequence set forth in any one of SEQ ID NOs: 793, 876, 1017, 1216, and 3291.

169. The pharmaceutical composition of any one of claims 163-168 or 155, wherein the IRES sequence has at least 99% sequence identity to a sequence set forth in any one of SEQ ID NOs: 793, 876, 1017, 1216, and 3291.

170. The pharmaceutical composition of any one of claims 163-169 or 155, wherein the IRES sequence comprise a sequence set forth in any one of SEQ ID NOs: 793, 876, 1017, 1216, and 3291.

171. A pharmaceutical composition comprising an oRNA comprising an IRES sequence having at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, and a pharmaceutically acceptable salt, buffer, diluent, or combination thereof, wherein the oRNA is capable of expressing a therapeutic protein in a T cell.

172. A pharmaceutical composition comprising an oRNA comprising an IRES sequence having at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, a T cell, and a pharmaceutically acceptable salt, buffer, diluent, or combination thereof.

173. A pharmaceutical composition comprising an oRNA comprising an IRES sequence having at least 85% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302, a transfer vehicle capable of delivering the oRNA into a T cell, and a pharmaceutically acceptable salt, buffer, diluent, or combination thereof.

174. The pharmaceutical composition of any one of claims 171-173 or 155, wherein the IRES sequence has at least 90% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

175. The pharmaceutical composition of any one of claims 171-174 or 155, wherein the IRES sequence has at least 95% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

176. The pharmaceutical composition of any one of claims 171-175 or 155, wherein the IRES sequence has at least 98% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

177. The pharmaceutical composition of any one of claims 171-176 or 155, wherein the IRES sequence has at least 99% sequence identity to a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

178. The pharmaceutical composition of any one of claims 171-177 or 155, wherein the IRES sequence comprise a sequence set forth in any one of SEQ ID NOs: 785, 823, 840, 857, 861, 862, 864, 983, 1023, 1168, 1169, 1171, 1179, 1192, 1284, 1287, 2285, 2742, 2777, 2778, 3283, 3290, 3293, and 3302.

179. A pharmaceutical composition comprising an oRNA comprising an IRES sequence having at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, and a pharmaceutically acceptable salt, buffer, diluent, or combination thereof, wherein the oRNA is capable of expressing a therapeutic protein in a T cell.

180. A pharmaceutical composition comprising an oRNA comprising an IRES sequence having at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, a T cell, and a pharmaceutically acceptable salt, buffer, diluent, or combination thereof.

181. A pharmaceutical composition comprising an oRNA comprising an IRES sequence having at least 85% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301, a transfer vehicle capable of delivering the oRNA into a T cell, and a pharmaceutically acceptable salt, buffer, diluent, or combination thereof.

182. The pharmaceutical composition of any one of claims 179-181, wherein the IRES sequence has at least 90% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

183. The pharmaceutical composition of any one of claims 179-182, wherein the IRES sequence has at least 95% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

184. The pharmaceutical composition of any one of claims 179-183, wherein the IRES sequence has at least 98% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

185. The pharmaceutical composition of any one of claims 179-184, wherein the IRES sequence has at least 99% sequence identity to a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

186. The pharmaceutical composition of any one of claims 179-185, wherein the IRES sequence comprise a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301.

187. The pharmaceutical composition of any one of claims 155-186, wherein the oRNA comprises the following elements, in the following order: (1) the IRES sequence; and (2) a coding sequence encoding a therapeutic protein, wherein elements (1) and (2) are operably linked to one another.

188. The pharmaceutical composition of any one of claims 155-187, wherein the IRES is capable of facilitating expression of the therapeutic protein encoded by a precursor RNA or polynucleotide in a cell.

189. The pharmaceutical composition of claim 188, wherein the cell is a T cell.

190. The pharmaceutical composition of claim 188, wherein the IRES is capable of facilitating expression of the therapeutic protein in the cell, such that the expression level of the protein in the cell is comparable to or higher than when a control IRES is used.

191. The pharmaceutical composition of claim 189, wherein the IRES is capable of facilitating expression of the therapeutic protein in the T cell, such that the expression level of the protein in the T cell is comparable to or higher than when a control IRES is used.

192. The pharmaceutical composition of any one of claims 155-189, wherein the therapeutic protein comprises a chimeric protein.

193. The pharmaceutical composition of claim 192, wherein the chimeric protein comprises a chimeric antigen receptor (CAR), a T-cell receptor (TCR), a B-cell receptor (BCR), an immune cell activation or inhibitory receptor, a recombinant fusion protein, a chimeric mutant protein, or a fusion protein, or a combination thereof.

194. The pharmaceutical composition of any one of claims 155-193, wherein the therapeutic protein comprises an antibody, a nanobody, a non-antibody protein, an immune modulatory ligand, a receptor, a structural protein, a growth factor ligand or receptor, a hormone or hormone receptor, a transcription factor, a checkpoint inhibitor or agonist, a Fc fusion protein, an anticoagulant, a blood clotting factor, a chaperone protein, an antimicrobial protein, a structural protein, a biochemical enzyme, a tight junction protein, a mitochondrial stress response, a cytoskeletal protein, a metal-binding protein, or a small molecule, or combinations thereof.

195. The pharmaceutical composition of claim 194, wherein the immune modulatory ligand comprises an interferon, a cytokine, a chemokine, or an interleukin.

196. The pharmaceutical composition of claim 194, wherein the structural protein comprises a channel protein or nuclear pore protein.

197. The pharmaceutical composition of any one of claims 187-196, wherein the coding sequence is codon-optimized.

198. The pharmaceutical composition of claim 197, wherein the coding sequence is optimized to lack at least one microRNA binding site present in an equivalent pre-optimized polynucleotide.

199. The pharmaceutical composition of claim 197 or 198, wherein the coding sequence is optimized to lack at least one RNA-editing susceptible site present in an equivalent pre-optimized polynucleotide.

200. The pharmaceutical composition of any one of claims 197-199, wherein the coding sequence is optimized to have G-C content that is between 50% and 70%.

201. The pharmaceutical composition of claim 200, wherein the coding sequence is optimized to have G-C content that is between 55% and 64%.

202. The pharmaceutical composition of any one of claims 155-201, wherein the oRNA is from about 0.1 to about 15 kilobases in length.

203. The pharmaceutical composition of any one of claims 155-202, having an in vivo duration of therapeutic effect in humans of at least 20 hours.

204. The pharmaceutical composition of any one of claims 155-203, having a functional half-life of at least 6 hours.

205. The pharmaceutical composition of any one of claims 155-204, having a duration of therapeutic effect in a human cell greater than or equal to that of an equivalent linear RNA polynucleotide comprising the same expression sequence.

206. The pharmaceutical composition of any one of claims 155-205, having an in vivo duration of therapeutic effect in human greater than that of an equivalent linear RNA polynucleotide having the same expression sequence.

207. The pharmaceutical composition of any one of claims 155-206, wherein the oRNA consists of natural nucleotides.

208. The pharmaceutical composition of any one of claims 155-207, wherein the pharmaceutical composition is formulated for delivery to a T cell via electroporation.

209. The pharmaceutical composition of any one of claims 155-208, wherein the oRNA is comprised in a nucleic acid expression vector.

210. The pharmaceutical composition of claim 209, wherein the nucleic acid expression vector is selected from the group consisting of a PCR product, a linearized plasmid, a non-linearized plasmid, a linearized minicircle, a non-linearized minicircle, a cosmid, a cDNA, or an artificial chromosome.

211. The pharmaceutical composition of any of claims 155, 157, 165, 173, and 181, wherein the transfer vehicle comprises a nanoparticle.

212. The pharmaceutical composition of claim 211, wherein the nanoparticle is a lipid nanoparticle, a core-shell nanoparticle, a biodegradable nanoparticle, a biodegradable lipid nanoparticle, a polymer nanoparticle, a polyplex or a biodegradable polymer nanoparticle.

213. The pharmaceutical composition of claim 211 or 212, wherein the nanoparticle is a lipid nanoparticle, a core-shell nanoparticle, or a biodegradable nanoparticle.

214. The pharmaceutical composition of any one of claims 211-213, wherein the nanoparticle comprises one or more cationic lipids, ionizable lipids, or poly p-amino esters, or combinations thereof.

215. The pharmaceutical composition of any one of claims 211-214, wherein the nanoparticle comprises one or more non-cationic lipids.

216. The pharmaceutical composition of any one of claims 211-215, wherein the nanoparticle comprises one or more PEG-modified lipids, structural lipids, helper lipids, polyglutamic acid lipids, or hyaluronic acid lipids or combinations thereof.

217. The pharmaceutical composition claim 216, wherein the one or more structural lipids comprise cholesterol.

218. The pharmaceutical composition of any one of claims 211-217, wherein the nanoparticle comprises arachidonic acid, leukotriene, oleic acid, or combinations thereof.

219. The pharmaceutical composition of any one of claims 214-218, wherein the molar ratio of the ionizable lipid in the transfer vehicle is from about 40 to about 60% of the total lipid present in the transfer vehicle.

220. The pharmaceutical composition of claim 214-219, wherein the molar ratio of the helper lipid in the transfer vehicle is from about 3.5% to about 14% of the total lipid present in the transfer vehicle.

221. The pharmaceutical composition of any one of claims 214-220, wherein molar ratio of the PEG-lipid in the transfer vehicle is from about 0.5% to about 5% of the total lipid present in the LNP.

222. The pharmaceutical composition of any one of claims 214-221, wherein structural lipid in the transfer vehicle is from about 28% to about 50% of the total lipid present in the transfer vehicle.

223. The pharmaceutical composition of any one of claims any one of claims 214-222, wherein the molar ratio of ionizable lipid:helper lipid:structural lipid:PEG-lipid is about 45:9:44:2, about 50:10:38.5:1.5, about 41:12:45:2, about 62:4:33:1, or about 53:5:41:1.

224. The pharmaceutical composition of any one of claims 214-223, wherein the nanoparticle has a lipid to phosphate (IL:P) ratio of about 3 to about 6, such as about 3, about 4, about 4.5, about 5, about 5.5, or about 6.

225. The pharmaceutical composition of any one of claim 155, or 157-224, wherein the transfer vehicle is formulated for endosomal release of the circular RNA polynucleotide.

226. The pharmaceutical composition of claim 211-225, wherein the nanoparticle comprises a targeting moiety operably connected thereto, wherein the targeting moiety mediates receptor-mediated endocytosis, endosome fusion, or direct fusion into T cells in the absence of cell isolation or purification.

227. The pharmaceutical composition of claim 226, wherein the targeting moiety comprises a small molecule, a scFv, a nanobody, a peptide, a cyclic peptide, a di- or tri-cyclic peptide, a minibody, a polynucleotide, an aptamer, an engineered scaffold protein, a heavy chain variable region, a light chain variable region, or a fragment thereof.

228. The pharmaceutical composition of any one of claims 155-227, wherein the transfer vehicle comprises a liposome, a dendrimer, a carbohydrate carrier, a glycan nanomaterial, a fusome, an exosome, or a combination thereof.

229. The pharmaceutical composition of any one of claims 155-228, wherein the T cell is a CD8+ cytotoxic T cell, a CD4+ helper T cell (Th), a regulatory T (Treg) cell, a memory T cell, or an innate-like T cell.

230. The pharmaceutical composition of claim 229, wherein the Th cell is a Th1 cell, a Th2 cell, a Th17 cell, a Th9 cell, a Tfh cell, or a Th22 cell.

231. The pharmaceutical composition of claim 229, wherein the memory T cell is a central memory T cell (Tcm), an effector memory T cell (Tem), a tissue-resident memory T cell (Trm), or a virtual memory T cell.

232. The pharmaceutical composition of claim 229, wherein the innate-like T cell is a natural killer T (NKT) cell, a mucosal-associated invariant T cell (MAIT), or a gamma delta T cell (γδ T cell).

233. A T cell comprising oRNA or precursor RNA polynucleotide of any one of claims 1-154 or the pharmaceutical composition of any one of claims 155-232.

234. The T cell of claim 233, wherein the T cell is a human T cell.

235. The T cell of claim 233 or 234, wherein the T cell is a CD8+ cytotoxic T cell, a CD4+ helper T cell (Th), a regulatory T (Treg) cell, a memory T cell, an innate-like T cell.

236. The T cell of claim 235, wherein the Th cell is a Th1 cell, a Th2 cell, a Th17 cell, a Th9 cell, a Tfh cell, or a Th22 cell.

237. The T cell of claim 235, wherein the memory T cell is a central memory T cell (Tcm), an effector memory T cell (Tem), a tissue-resident memory T cell (Trm), or a virtual memory T cell.

238. The T cell of claim 235, wherein the innate-like T cell is a natural killer T (NKT) cell, a mucosal-associated invariant T cell (MAIT), or a gamma delta T cell (γδ T cell).

239. A eukaryotic cell comprising the oRNA or precursor RNA polynucleotide of any one of claims 1-154, or the pharmaceutical composition of any one of claims 155-228.

240. The eukaryotic cell of claim 239, wherein the eukaryotic cell is a human cell.

241. The eukaryotic cell of claim 239 or 240, wherein the eukaryotic cell is an immune cell.

242. The eukaryotic cell of claim of any one of claims 239-241, wherein the eukaryotic cell is a T cell, dendritic cell, macrophage, B cell, neutrophil, or basophil.

243. A prokaryotic cell comprising the oRNA or precursor RNA polynucleotide of any one of claims 1-154, or the pharmaceutical composition of any one of claims 155-228.

244. A method of expressing a therapeutic protein in a cell, comprising contacting the cell with the oRNA or precursor RNA polynucleotide of any one of claims 1-154, or the pharmaceutical composition of any one of claims 155-232, thereby expressing the therapeutic protein in the cell.

245. A method of expressing a protein from an oRNA molecule, comprising providing oRNA comprising an IRES selected from a sequence set forth in any one of SEQ ID NOS: 14067-24829 or having a consensus sequence set forth in any one of SEQ ID NOs: 24867-24892 adjacent to a coding sequence.

246. A method of expressing a protein from an oRNA molecule in a T cell, comprising providing the T cell with an oRNA comprising an IRES selected from a sequence set forth in any one of SEQ ID NOs: 793, 876, 1017, 1216, and 3291 adjacent to a coding sequence.

247. A method of expressing a protein from an oRNA molecule in a T cell, comprising providing the T cell with an oRNA comprising an IRES selected from a sequence set forth in any one of SEQ ID NOs: 75, 77, 137, 532, 566, 580, 648, 693, 752, 787, 791, 820, 839, 843, 852, 863, 871, 874, 922, 959, 984, 1015, 1026, 1041, 1047, 1059, 1068, 1134, 1177, 1178, 1180, 1189, 1193, 1198, 1263, 1276, 1280, 1282, 2601, 2615, 2616, 2617, 2618, 2627, 2667, 2681, 2746, 2758, 3284, 3285, 3289, 3292, 3294, 3295, 3296, 3297, 3298, 3299, and 3301 adjacent to a coding sequence.

248. A method of delivering an oRNA or a RNA precursor thereof to a T cell, comprising the oRNA or precursor RNA polynucleotide of any one of claims 1-154, or the pharmaceutical composition of any one of claims 155-232.

249. The method of claim 248, wherein the delivery to the T cell is performed using electroporation.

250. A method of treating a subject having a disease or disorder, the method comprising administering the pharmaceutical composition of any one of claims 155-232 to the subject.

251. A method of treating a subject having a disease or disorder, the method comprising administering the T cell of any one of claims 233-238 to the subject.

252. The method of any one of claim 250 or 251, wherein the disease or disorder is a cancer.

253. The method of any one of claim 250 or 251, wherein the disease or disorder is an autoimmune disease or disorder.

254. The method of any one of claims 250-253, wherein the subject is a human.

255. A method for identifying a translation initiation element (TIE) sequence capable of driving protein expression comprising:
a. obtaining a pool of RNA molecules comprising TIE,
b. transfecting the RNA molecules into a cell comprising one or more ribosomes,
c. lysing the cell and adding the content of the lysed cell into a gradient to fractionate based on a ribosomal load, and
d. selecting the RNA and TIE combination capable of loading the RNA into the ribosome.

256. The method of claim 255, wherein the RNA molecule comprises a circular RNA or a linear RNA.

257. The method of claim 255, wherein the TIE is an internal ribosome entry site (IRES).

258. The method of claim 255, wherein the cell is a mammalian cell.

259. The method of claim 255, wherein the gradient is a sucrose gradient.

260. The method of claim 255, further comprising centrifuging the content of the lysed cell before fractionation.

261. A method for identifying a circular RNA sequence capable of driving protein expression comprising:
a. obtaining a pool of RNA molecules comprising TIE,
b. transfecting the RNA molecules into a cell comprising one or more ribosomes,
c. lysing the cell and adding the content of the lysed cell into a gradient to fractionate based on a ribosomal load, and
d. selecting the RNA and TIE combination capable of loading the RNA into the ribosome.

262. The method of claim 255 for identifying a TIE sequence capable of driving protein expression in a circular RNA comprising the TIE operably linked to a sequence encoding said protein.

263. The method of claim 262 for identifying a TIE sequence capable of high translation efficiency as compared to a TIE comprising SEQ ID NO: 3282 under comparable conditions.
